(19) United States
(12) Patent Application Publication
(10) Pub. No.: US 2026/0158159 A1
(43) Pub. Date: Jun. 11, 2026

(54) Fc-ACYLATED POLYPEPTIDE CONJUGATES

(71) Applicant: ELI LILLY AND COMPANY, Indianapolis, IN (US)

(72) Inventors: Robert Andrew BROWN, Carmel, IN (US); Guillermo S. CORTEZ, Indianapolis, IN (US); Paul Joseph KLEINDL, Lebanon, IN (US); Dahamsondage Nuwan KONDASINGHE, Indianapolis, IN (US); Daniel LOPES, Indianapolis, IN (US); Faiz Ahmad MOHAMMED, Carmel, IN (US); Avinash MUPPIDI, Carmel, IN (US); David John STOKELL, Indianapolis, IN (US); Yan WANG, Carmel, IN (US)

(21) Appl. No.: 19/279,475

(22) Filed: Jul. 24, 2025

Related U.S. Application Data
(60) Provisional application No. 63/675,095, filed on Jul. 24, 2024.

(51) Int. Cl. A61K 47/68; A61P 3/04; C07K 14/00

(52) U.S. Cl. CPC A61K 47/6811; A61K 47/68; A61P 3/04; C07K 14/001

(57) ABSTRACT

Compounds comprising therapeutic polypeptide Fc conjugates are provided that have activity at one or more of the GIP, GLP-1 and glucagon receptors. The compounds have structural features resulting in activity and extended duration of action at one or more of these receptors. Methods also are provided for treating diseases and/or conditions such as obesity, chronic weight management, and type 2 diabetes mellitus.

### SEQUENCE LISTING

The present application is being filed along with a Sequence Listing in ST.26 XML format. The Sequence Listing is provided as a file titled “30783_WO_SL.xml” created Jul. 21, 2025 and is 5,378,048 bytes in size. The Sequence Listing information in the ST.26 XML format is incorporated herein by reference in its entirety.

### FIELD

This disclosure pertains to novel compounds, specifically therapeutic polypeptide Fc conjugates, designed to exhibit extended therapeutic effects on, for example, glucose-dependent insulinotropic polypeptide (GIP), glucagon-like peptide-1 (GLP-1), and/or glucagon (GCG) receptors. These conjugates incorporate structural modifications that optimize receptor activity and enhance the duration of action, offering improved pharmacological profiles for therapeutic applications.

### BACKGROUND

The prevalence of diabetes mellitus continues to increase, representing a chronic condition marked by persistent hyperglycemia due to disruptions in insulin secretion, insulin function, or both. Type 2 diabetes mellitus (T2DM) is the most common form, accounting for approximately 90% of cases. In T2DM, elevated blood glucose levels result from a combination of impaired insulin secretion and insulin resistance.

Unmanaged diabetes in individuals may contribute to various health complications that affect both quality of life and overall survival. Excess weight is a significant risk factor for T2DM, and a large proportion of individuals with T2DM, approximately 90%, are classified as overweight or living with obesity. Research suggests that reducing body adiposity may contribute to improvements in health conditions associated with obesity.

The standard approach to managing T2DM involves lifestyle modifications, including diet and exercise, along with pharmacologic interventions such as oral and injectable incretin-based therapies and insulin treatments. Injectable incretin-based therapies currently available include GLP-1 receptor agonists, such as dulaglutide and semaglutide, as well as the dual GIP and GLP-1 receptor agonist, tirzepatide. Therapeutics containing semaglutide and tirzepatide have also been approved to support weight reduction and long-term weight management in individuals who are overweight or living with obesity. Research has been conducted on additional compounds stated to have dual agonist activity at the GIP and GLP-1 receptors, such as those described in US20200024322, as well as compounds stated to have triple agonist activity at the GIP, GLP-1 and glucagon receptors, such as those described in US20240270821. Additionally, insulin therapy remains a cornerstone of diabetes management, particularly for individuals with advanced disease or significant insulin deficiency.

However, currently available injectable incretin-based and insulin therapies generally require at least weekly administration. Many individuals may prefer options requiring fewer injections, and expanding treatment availability to include less frequent administration could enhance patient adherence, acceptance, and overall treatment success.

Several approaches for enhancing duration of action of protein or peptide therapeutics have been suggested. Such approaches include conjugation of the therapeutic to an Fc region of an antibody (see, for example, WO2016/131893) and lipidation with a fatty acid moiety (see, for example, US2020/0283492). Further, A. Zaykov, et al (2024), RSC Chem. Biol. describes the effects of a combination of lipidation and Fc-conjugation on the pharmacokinetic and pharmacodynamic profile of an insulin molecule.

Nevertheless, there remains a need for innovative treatment options that provide an extended duration of therapeutic action while reducing the frequency of administration compared to currently available therapies. Addressing this need could enhance patient adherence, improve treatment outcomes, and offer a more convenient approach to disease management.

### SUMMARY

In a first aspect, the present disclosure provides a compound of the formula:

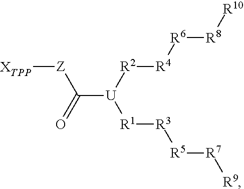

or a pharmaceutically acceptable salt thereof, wherein:
X_{TPP} is a therapeutic polypeptide conjugated to a fatty acid moiety; Z is O, C(O), NH, C₁ to C₃₀ alkyl, (OCH₂CH₂)ₘ, [C(O)NH—CH₂CH₂OCH₂]ₘ, an amino acid, a peptide, or a combination thereof, wherein m is an integer between 1-30; U is C₁ to C₅ alkyl, N, phenyl, or phenyl carbonyl, or a combination thereof; R¹ and R² are independently absent, a covalent bond, or C₁ to C₅ alkyl;
R³ and R⁴ are independently absent or an amide; R⁵ and R⁶ are independently absent or selected from the group consisting of C₁ to C₅ alkyl or phenyl, which is optionally substituted with one or more of NH₂, CH₂NH₂, and CH₂CH₂NH₂, CH₂O(CH₂CH₂O)ₙ, wherein n is an integer between 1-5; and R⁷ and R⁸ are independently absent or selected from the group consisting of:

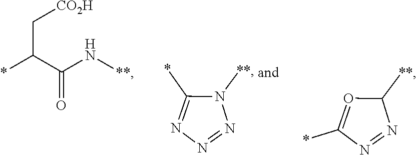

wherein (*) is a connection point to R⁹ and R¹⁰ and (**) is the connection point to R⁵ or R⁶; and R⁹ and R¹⁰ are each an antibody or fragment thereof. In an embodiment, the therapeutic polypeptide comprises an agonist at one or more of the GIP, GLP-1 and glucagon receptors.

In a second aspect, the present disclosure provides a compound of the formula:

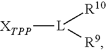

or a pharmaceutically acceptable salt thereof, wherein:
X_{TPP}, is a therapeutic polypeptide, comprising:

(SEQ ID NO: 1219)
YAibEGTX₆TSDX₁₀X₁₁X₁₂X₁₃LX₁₅X₁₆KAQX₂₀X₂₁FIX₂₄X₂₅LX₂₇
X₂₈X₂₉X₃₀X₃₁X₃₂X₃₃X₃₄X₃₅X₃₆X₃₇X₃₈X₃₉X₄₀X₄₁

wherein: X₆ is αMeF(2F), F, or αMeF; X₁₀ is 4-Pal, Y, or V; X₁₁ is Aib or S; X₁₂ is I or S, X₁₃ is L or αMeL; X₁₅ is D or E; X₁₆ is E or Orn; X₂₀ is Aib, αMeL, or Iva; X₂₁ is E, Q, D, or Orn; X₂₄ is K, Q, E, or D-Glu; X₂₅ is αMeY or Y; X₂₇ is I, L, or V; X₂₈ K, E or A; X₂₉ is Aib, G, or Q; X₃₀ is G or S; X₃₁ is P, G, E, or Orn; X₃₂ is absent, K, S, or P; wherein if X₃₂ is S or P, then X₃₃ is S; wherein if X₃₃ is S, then is X₃₄ is G or Aib; wherein if X₃₄ is G or Aib, then X₃₅ is absent, A, or Orn; wherein if X₃₅ is A or Orn, then X₃₆ is absent or P; wherein if X₃₆ is P, then X₃₇ is absent or P; wherein if X₃₇ is P, then X₃₈ is absent or P; wherein if X₃₈ is P, then X₃₉ is absent, S, Orn, or G; wherein if X₃₉ is S, Orn, or G, then X₄₀ is absent, K, or G; wherein if X₄₀ is K or G, then X₄₁ is absent, S, or G; wherein if X₃₂ absent or K, then X₃₃ through X₄₁ are also absent; wherein if X₃₅ is absent, then X₃₆ through X₄₁ are also absent; wherein if X₃₆ is absent, then X₃₇ through X₄₁ are also absent; wherein if X₃₇ is absent, then X₃₈ through X₄₁ are also absent; wherein if X₃₈ is absent, then X₃₉ through X₄₁ are also absent; wherein if X₃₉ is absent, then X₄₀ and X₄₁ are also absent; wherein if X₄₀ is absent, then X₄₁ is absent; L is a linker, conjugated to each of R⁹, R¹⁰, and X_{TPP}; and R⁹ and R¹⁰ are each an antibody or fragment thereof.

In an embodiment, the therapeutic polypeptide has dual agonist activity at the GIP and GLP-1 receptors.

In a third aspect, the present disclosure provides a compound of the formula:

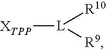

or a pharmaceutically acceptable salt thereof, wherein:
X_{TPP}, is a therapeutic polypeptide, comprising:

(SEQ ID NO: 1220)
X₁X₂X₃X₄TX₆TSDX₁₀X₁₁X₁₂X₁₃LX₁₅X₁₆KAQX₂₀X₂₁FIX₂₄X₂₅LX₂₇X₂₈X₂₉
X₃₀X₃₁X₃₂X₃₃X₃₄X₃₅X₃₆X₃₇X₃₈X₃₉X₄₀X₄₁

wherein: X₁ is H, NMeY, or Y; X₂ is Ac4c, Aib, αMeS, Iva, or D-Ala; X₃ is Q. E, or H; X₄ is G or D-Ala; X₆ is αMeF(2F), F, or αMeF; X₁₀ is 4-Pal, Y, or V; X₁₁ is Aib, S, or αMeS; X₁₂ is I or S; X₁₃ is L or αMeL; X₁₅ is D or E; X₁₆ is E or Orn; X₂₀ is Aib, αMeL, Iva, or αMe4Pal; X₂₁ is E, Q, D, or Orn; X₂₄ is K, Q, E, D-Glu, or D-Gln; X₂₅ is αMeY or Y; X₂₇ is I, L, or V; X₂₈ K, E or A; X₂₉ is Aib, G, or Q, X₃₀ is G or S; X₃₁ is P, G, E, or Orn; X₃₂ is absent, S or P; wherein if X₃₂ is S or P, then X₃₃ is S; wherein if X₃₃ is S, then is X₃₄ is G or Aib; wherein if X₃₄ is G or Aib, then X₃₅ is absent or A or Orn; wherein if X₃₅ is A or Orn, then X₃₆ is absent or P; wherein if X₃₆ is P, then X₃₇ is absent or P; wherein if X₃₇ is P, then X₃₈ is absent or P; wherein if X₃₈ is P, then X₃₉ is absent or S, Orn, or G; wherein if X₃₉ is S, Orn, or G, then X₄₀ is absent, K, or G; wherein if X₄₀ is K or G, then X₄₁ is absent or S or G; wherein if X₃₂ is absent or K, then X₃₃ through X₄₁ are also absent; wherein if X₃₅ is absent, then X₃₆ through X₄₁ are also absent; wherein if X₃₆ is absent, then X₃₇ through X₄₁ are also absent; wherein if X₃₇ is absent, then X₃₈ through X₄₁ are also absent; wherein if X₃₈ is absent, then X₃₉ through X₄₁ are also absent; wherein if X₃₉ is absent, then X₄₀ and X₄₁ are also absent; wherein if X₄₀ is absent, then X₄₁ is absent; L is a linker, conjugated to each of R⁹, R¹⁰, and X_{TPP}; and R⁹ and R¹⁰ are an antibody or an antibody fragment thereof.

In an embodiment, the therapeutic polypeptide has triple agonist activity the GIP, GLP-1 and glucagon receptors.

In a fourth aspect, the present disclosure provides a pharmaceutical composition comprising a compound described herein, or a pharmaceutically acceptable salt thereof, and at least one pharmaceutically acceptable carrier, diluent, or excipient.

In a fifth aspect, the present disclosure provides a method of treating a disease or condition, the method comprising a step of administering to an individual in need thereof an effective amount of a compound described herein, or a pharmaceutically acceptable salt thereof.

### DESCRIPTION OF THE DRAWINGS

FIG. 1 shows the molecular structure for Compound 164 (SEQ ID NO:622).

FIG. 2 shows the molecular structure for Compound 1 (SEQ ID NO:459).

FIG. 3 shows the molecular structure for Compound 32 (SEQ ID NO:490).

FIG. 4 shows the molecular structure for Compound 47 (SEQ ID NO:505).

FIG. 5 shows the molecular structure for Compound 127 (SEQ ID NO:585).

FIG. 6 shows the molecular structure for Compound 129 (SEQ ID NO: 587).

FIG. 7 shows the molecular structure for Compound 136 (SEQ ID NO:594).

FIG. 8 shows the molecular structure for Compound 158 (SEQ ID NO:616).

FIG. 9 shows the molecular structure for Compound 168 (SEQ ID NO:626).

FIG. 10 shows the molecular structure for Compound 169 (SEQ ID NO:627).

FIG. 11 shows the molecular structure for Compound 170 (SEQ ID NO:628).

FIG. 12 shows the molecular structure for Compound 172 (SEQ ID NO:630).

FIG. 13 shows the molecular structure for Compound 173 (SEQ ID NO:631).

FIG. 14 shows the molecular structure for Compound 181 (SEQ ID NO:639).

FIG. 15 shows the molecular structure for Compound 183 (SEQ ID NO:641).

FIG. 16 shows the molecular structure for Compound 184 (SEQ ID NO:642).

FIG. 17 shows the molecular structure for Compound 185 (SEQ ID NO:643).

FIG. 18 shows the molecular structure for Compound 187 (SEQ ID NO:645).

FIG. 19 shows the molecular structure for Compound 188 (SEQ ID NO:646)

FIG. 20 shows the molecular structure for Compound 204 (SEQ ID NO:662).

FIG. 21 shows the molecular structure for Compound 479 (SEQ ID NO:1099).

FIG. 22 shows the molecular structure for Compound 480 (SEQ ID NO:698).

FIG. 23 shows the molecular structure for Compound 481 (SEQ ID NO:699).

FIG. 24 shows the molecular structure for Compound 482 (SEQ ID NO: 700).

FIG. 25 shows the molecular structure for Compound 336 (SEQ ID NO:792).

FIG. 26 shows the molecular structure for Compound 387 (SEQ ID NO:843).

FIG. 27 shows the molecular structure for Compound 403 (SEQ ID NO: 859).

FIG. 28 shows the molecular structure for Compound 413 (SEQ ID NO: 869).

FIG. 29 shows the molecular structure for Compound 433 (SEQ ID NO: 889).

FIG. 30 shows the molecular structure for Compound 435 (SEQ ID NO:891).

FIG. 31 shows the molecular structure for Compound 444 (SEQ ID NO:900).

FIG. 32 shows the molecular structure for Compound 483 (SEQ ID NO: 1100).

FIG. 33 shows the molecular structure for Compound 484 (SEQ ID NO: 1101).

FIG. 34 shows the molecular structure for Compound 485 (SEQ ID NO: 1102).

FIG. 35 shows the molecular structure for Compound 456 (SEQ ID NO:912).

FIG. 36 shows the molecular structure for Compound 486 (SEQ ID NO: 1103).

FIG. 37 shows the molecular structure for Compound 487 (SEQ ID NO: 1104).

FIG. 38 shows the molecular structure for Compound 459 (SEQ ID NO:915).

FIG. 39 shows the molecular structure for Compound 493 (SEQ ID NO:1110).

FIG. 40 shows the molecular structure for Compound 555 (SEQ ID NO: 1172).

FIG. 41 shows the molecular structure for Compound 556 (SEQ ID NO: 1173).

FIG. 42 shows the molecular structure for Compound 557 (SEQ ID NO: 1174).

FIG. 43 shows the molecular structure for Compound 558 (SEQ ID NO:1175).

FIG. 44 shows weekly administration of Compound 170 (SEQ ID NO:628) dose-dependently reduced body weight in diet-induced obese mice.

FIG. 45 shows weekly administration of Compound 172 (SEQ ID NO:630) dose-dependently reduced body weight in diet-induced obese mice.

FIG. 46 shows weekly administration of Compound 387 (SEQ ID NO:843) dose-dependently reduced body weight in diet-induced obese mice.

FIG. 47 shows weekly administration of Compound 456 (SEQ ID NO:912) dose-dependently reduced body weight in diet-induced obese mice.

### DETAILED DESCRIPTION

The present disclosure pertains to methods and compositions comprising therapeutic polypeptide Fc conjugates to facilitate the delivery of therapeutic polypeptides to patients. These conjugates comprise of a therapeutic polypeptide covalently linked to an antibody, or a fragment thereof, such as an Fc region, via a linker. Notably, the conjugation reaction between the therapeutic polypeptide, the disclosed linkers, and the antibody or fragment thereof occurs under relatively mild conditions, eliminating the need for protective measures during the reaction process.

Surprisingly, by attaching an acylated therapeutic polypeptide to an antibody or fragment thereof, such as an Fc region, with to a linker disclosed herein, the duration of action of the therapeutic polypeptide is improved and/or lengthened to a greater extent than would be expected through the use of either acylation or conjugation to an Fc region on their own. The attachment of the therapeutic polypeptide to the linker and Fc region may increase the duration of action of the therapeutic polypeptide by one or more of increasing the hydrodynamic size of the overall moiety, FcRn binding and/or recycling, and preventing the breakdown of the therapeutic polypeptide. The reaction conditions for the conjugation reaction between the linkers disclosed herein and antibodies or antibody fragments, such as Fc regions, are relatively mild, so the therapeutic polypeptides do not need to be modified to be protected during the reaction. Acylation of the therapeutic polypeptide may further increase the duration of action of the therapeutic polypeptide by one or more of promoting binding to serum albumin. The duration of action of the therapeutic polypeptide may be further increased in certain embodiments through the inclusion of structural features that improve the proteolytic stability of the therapeutic polypeptide.

### Therapeutic Polypeptide Fc Conjugates

The present disclosure provides multiple different embodiments of compounds comprising features resulting in increased duration of action.

In certain embodiments, the present disclosure provides a compound of Formula I:

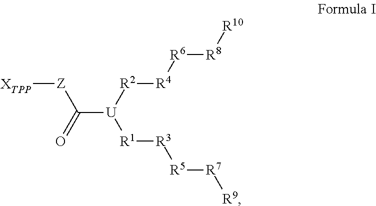

or a pharmaceutically acceptable salt thereof, wherein:
a therapeutic polypeptide, X_{TPP}, is conjugated to a fatty acid moiety; Z is a linker; U is C₁ to C₅ alkyl, N, phenyl, or phenyl carbonyl, or a combination thereof; R¹ and R² are independently absent, a covalent bond, or C₁ to C₅ alkyl; R³ and R⁴ are independently absent or an amide; R⁵ and R⁶ are independently absent or selected from the group consisting of C₁ to C₅ alkyl or phenyl, which is optionally substituted with one or more of NH₂, CH₂NH₂, and CH₂CH₂NH₂, CH₂O(CH₂CH₂O)ₙ, wherein n is an integer between 1-5; and R⁷ and R⁸ are independently absent or selected from the group consisting of:

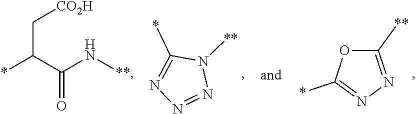

wherein (*) is a connection point to R⁹ or R¹⁰ and (**) is a connection point to R⁵ or R⁶; and R⁹ and R¹⁰ are each an antibody or fragment thereof.

In certain embodiments, Z comprises one or more of: O, C(O), NH, C₁ to C₃₀ alkyl, (OCH₂CH₂)ₘ, [C(O)NH—CH₂CH₂OCH₂]ₘ, (2-[2-(2-amino-ethoxy)-ethoxy]-acetyl)ₘ, an amino acid, a peptide, or a combination thereof, wherein m is an integer between 1-30.

In certain embodiments, R¹ and R² are the same. In certain embodiments, R³ and R⁴ are the same. In certain embodiments; R⁵ and R⁶ are the same. In certain embodiments, R⁷ and R⁸ are the same. In certain embodiments, R⁹ and R¹⁰ are Fc regions. In certain embodiments, R⁹ and R¹⁰ are the same.

In certain embodiments, the therapeutic polypeptide has agonist activity at one or more of the GIP, GLP-1 and/or glucagon receptors.

In some embodiments, the present disclosure provides a compound of Formula I, wherein R⁷ and R⁸ are:

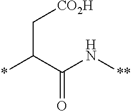

In some embodiments, R⁵ and R⁶ are each a C₂ alkyl substituted with NH₂. In some embodiments, R³ and R⁴ are each an amide. In some embodiments, R⁵ and R⁶ are each a C₂ alkyl. In some embodiments, U is N or a phenyl.

In some embodiments, the present disclosure provides a compound of Formula I-A:

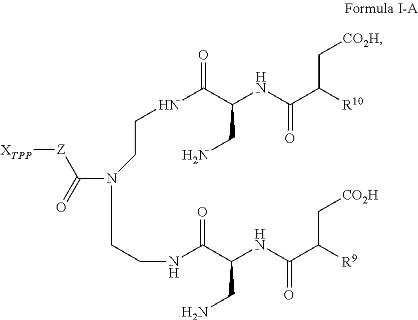

or a pharmaceutically acceptable salt thereof.

In some embodiments, the present disclosure provides a compound of Formula I, wherein R⁷ and R⁸ are:

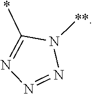

In some embodiments, R⁵ and R⁶ are each a phenyl substituted with (OCH₂CH₂)ₘ, wherein m is 3. In some embodiments, R³ and R⁴ are each an amide. In some embodiments, R¹ and R² are each a C₂ alkyl. In some embodiments, U is N or a phenyl.

In some embodiments, the present disclosure provides a compound of Formula I-B:

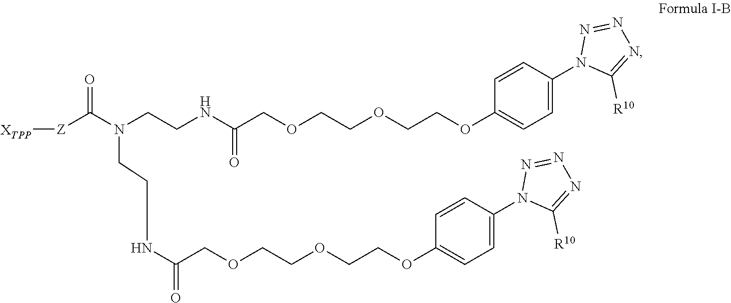

or a pharmaceutically acceptable salt thereof.

In some embodiments the present disclosure provides a compound of Formula I, wherein R⁷ and R⁸ are:

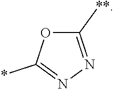

In some embodiments, R⁵ and R⁶ are each a phenyl substituted with (OCH₂CH₂)ₘ, wherein m is 3. In some embodiments, R³ and R⁴ are each an amide. In some embodiments, R¹ and R² are each a C₂ alkyl. In some embodiments, U is N or a phenyl.

In some embodiments, the present disclosure provides a compound of Formula I-C:

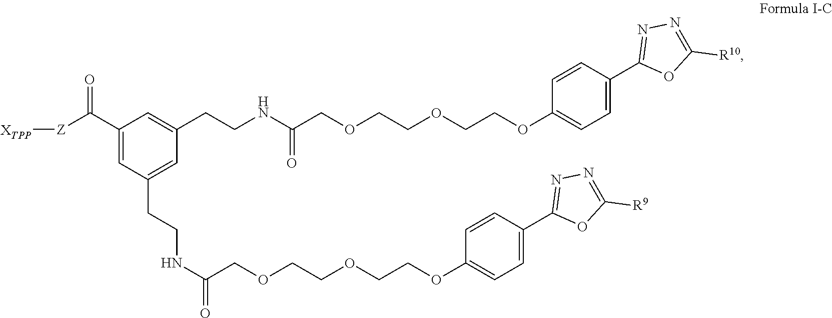

or a pharmaceutically acceptable salt thereof.

In some embodiments of compounds of Formula I, R⁷ and R⁸ are each absent. In some embodiments, R⁵ and R⁶ are each C₁ alkyl. In some embodiments, R³ and R⁴ are each an amide. In some embodiments, R¹ and R² are each a C₂ alkyl. In some embodiments, U is N or a phenyl.

In some embodiments, the present disclosure provides a compound of Formula I-D:

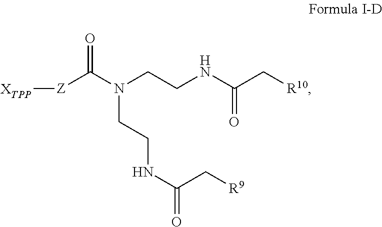

or a pharmaceutically acceptable salt thereof.

In some embodiments, the present disclosure provides a compound of Formula I-E:

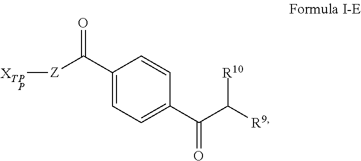

or a pharmaceutically acceptable salt thereof.

In certain embodiments, the present disclosure provides a compound of Formula II:

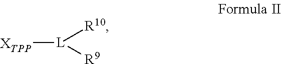

or a pharmaceutically acceptable salt thereof, wherein:
X_{TPP} is a therapeutic polypeptide L is a linker conjugated to the therapeutic polypeptide, R⁹, and R¹⁰; and R⁹ and R¹⁰ are each an antibody or antibody fragment thereof. In certain embodiments, R⁹ and R¹⁰ are each an Fc region.

In some embodiments, the therapeutic polypeptide has dual agonist activity at the GIP and GLP-1 receptors. In some embodiments, the therapeutic polypeptide has tri agonist activity at the GIP, GLP-1 and glucagon receptors.

In certain embodiments, the present disclosure provides a compound of Formula III:

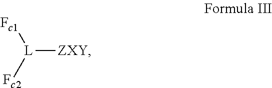

or a pharmaceutically acceptable salt thereof, wherein:
Fc₁ and Fc₂ are absent or are each an Fc region; L is a linker; Z is a functional group of a small molecule, an amino acid polymer, or a combination thereof; X is a therapeutic polypeptide, wherein the therapeutic polypeptide is optionally conjugated to a fatty acid, Y.

In certain embodiments, the present disclosure provides a compound of Formula IV:

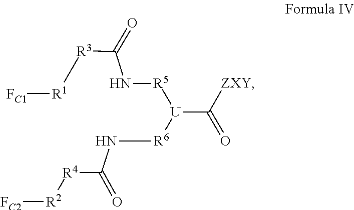

or a pharmaceutically acceptable salt thereof, wherein:
R¹ and R² are absent or independently selected from the group consisting of:

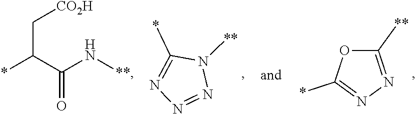

wherein:
(*) is a connection point to a sulfur atom in a cysteine residue of Fc₁ or Fc₂, and (**) is the connection point to R³ or R⁴; R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In some embodiments, the present disclosure provides a compound of Formula IV-A:

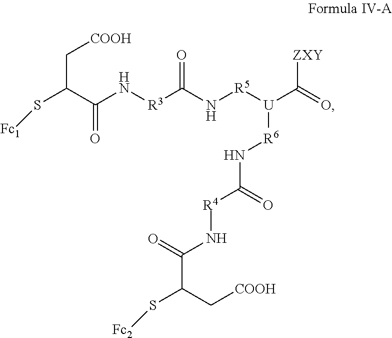

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In another embodiment, the present disclosure provides a compound of Formula IV-A comprising:

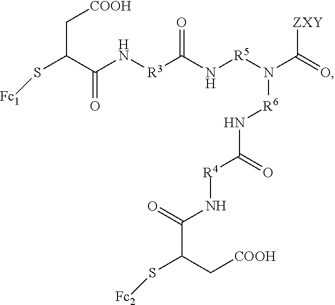

or a pharmaceutically acceptable salt thereof, wherein:
R₃ and R₄ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30, X is a therapeutic polypeptide; Y is absent or a fatty acid; and Fc₁ and Fc₂ are independently an Fc region.

In yet another embodiment, the present disclosure provides a compound of Formula IV-A comprising:

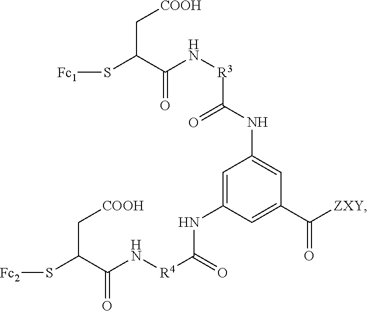

or a pharmaceutically acceptable salt thereof, wherein:
R₃ and R₄ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30, X is a therapeutic polypeptide; Y is absent or a fatty acid; and Fc₁ and Fc₂ are independently an Fc region.

In some embodiments, the present disclosure provides a compound of Formula IV-B:

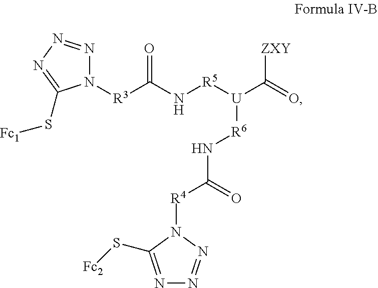

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In another embodiment, the present disclosure provides a compound of Formula IV-B comprising:

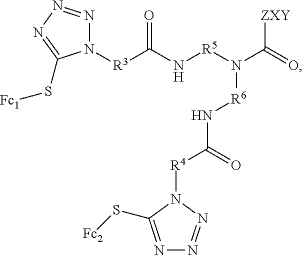

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In yet another embodiment, the present disclosure provides a compound of Formula IV-B comprising:

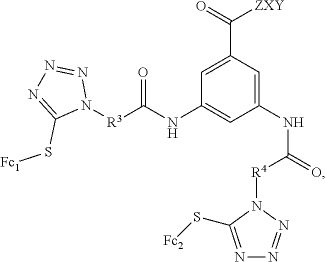

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In some embodiments, the present disclosure provides a compound of Formula IV-C:

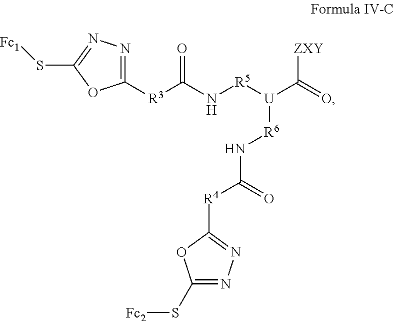

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In another embodiment, the present disclosure provides a compound of Formula IV-C comprising:

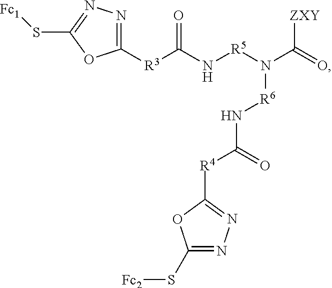

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In yet another embodiment, the present disclosure provides a compound of Formula IV-C comprising:

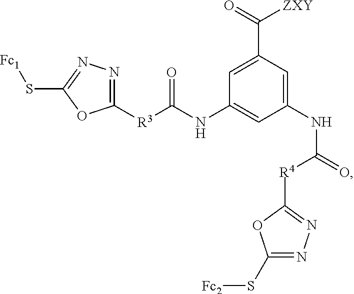

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In some embodiments, the present disclosure provides a compound of Formula IV-D:

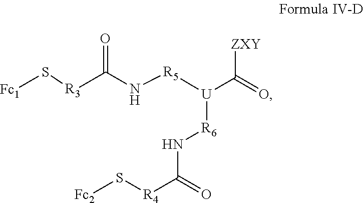

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In another embodiment, the present disclosure provides a compound of Formula IV-D comprising:

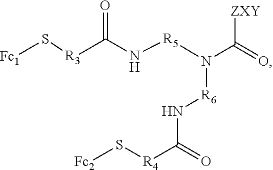

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

In yet another embodiment, the present disclosure provides a compound of Formula IV-D comprising:

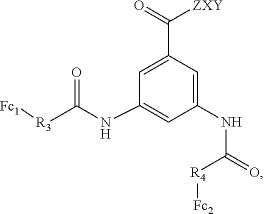

or a pharmaceutically acceptable salt thereof, wherein:
R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region.

### Intermediate Therapeutic Polypeptide Conjugates

The present disclosure provides intermediate compounds comprising therapeutic polypeptides conjugated to linkers for subsequent conjugation to an antibody or fragment thereof, e.g., an Fc region. Non-limiting examples of such intermediate compounds are provided below.

### Maleimide Intermediate Compounds

In some embodiments, the present disclosure provides maleimide intermediate compounds of Formula IV-A comprising:

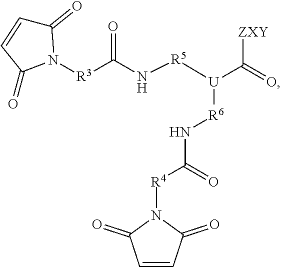

or a pharmaceutically acceptable salt thereof, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

In another embodiment, the present disclosure provides maleimide intermediate compounds of Formula IV-A comprising:

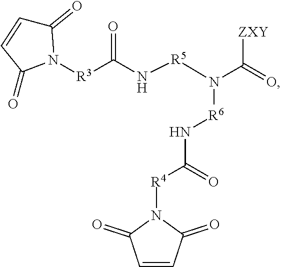

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

In yet another embodiment, the present disclosure provides maleimide intermediate compounds of Formula IV-A comprising:

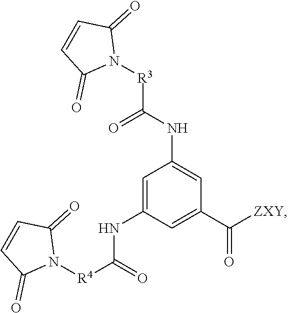

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30, X is a therapeutic polypeptide; and Y is absent or a fatty acid.

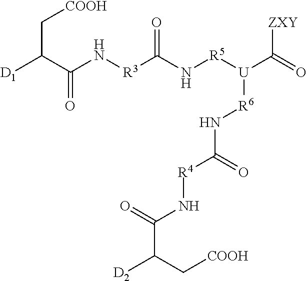

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide, Y is absent or a fatty acid; and D₁ and D₂ independently comprise an amide, amine, imine, sulfonamide, thiourea, urea, thioether, or combinations thereof.

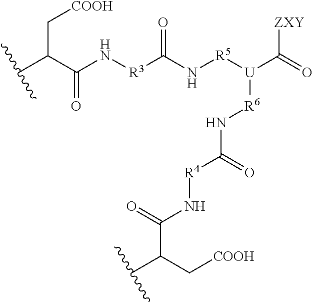

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

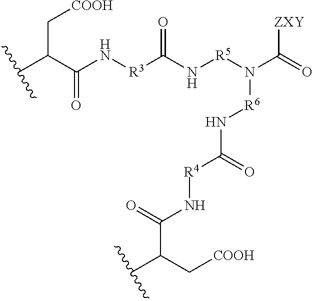

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

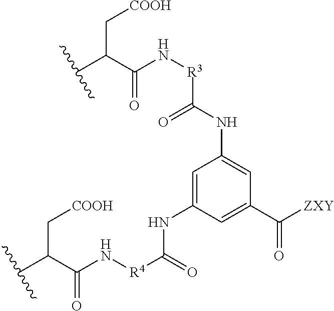

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

### Tetrazole Intermediate Compounds

In some embodiments, the present disclosure provides tetrazole intermediate compounds of Formula IV-B comprising:

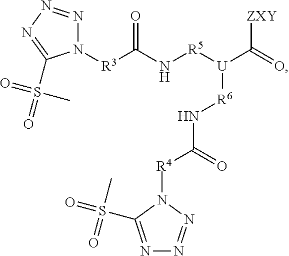

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

In another embodiment, the present disclosure provides tetrazole intermediate compounds of Formula IV-B comprising:

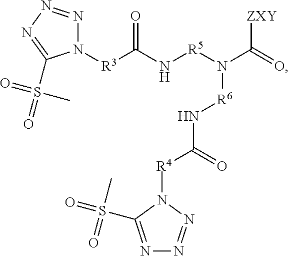

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

In yet another embodiment, the present disclosure provides tetrazole intermediate compounds of Formula IV-B comprising:

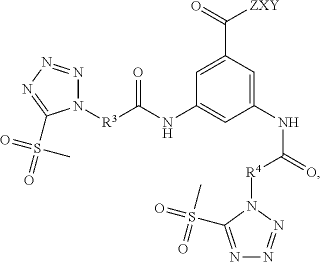

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

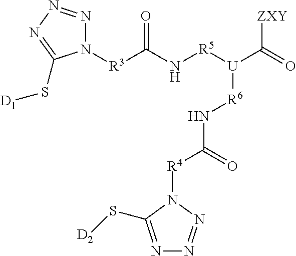

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide, Y is absent or a fatty acid; and D₁ and D₂ independently comprise an amide, amine, imine, sulfonamide, thiourea, urea, thioether, or combinations thereof.

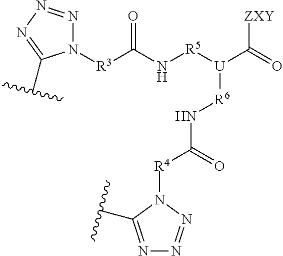

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

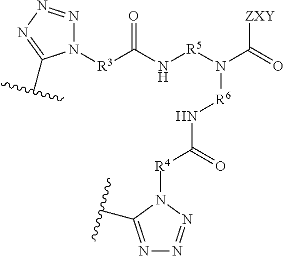

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

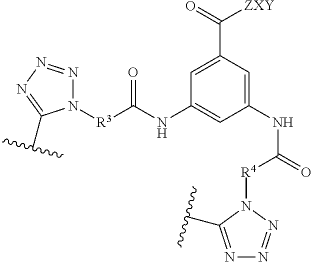

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

### Oxidiazole Intermediate Compounds

In some embodiments, the present disclosure provides tetrazole intermediate compounds of Formula IV-C comprising:

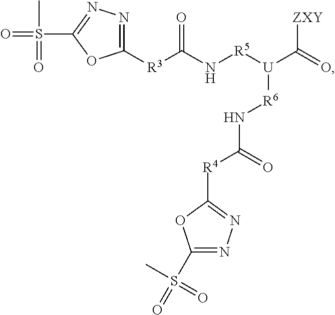

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

In another embodiment, the present disclosure provides tetrazole intermediate compounds of Formula IV-C comprising:

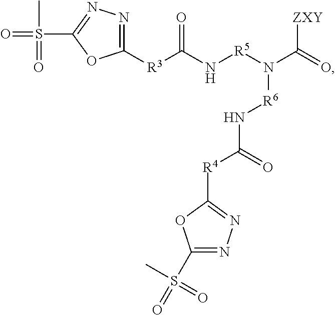

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

In yet another embodiment, the present disclosure provides tetrazole intermediate compounds of Formula IV-C comprising:

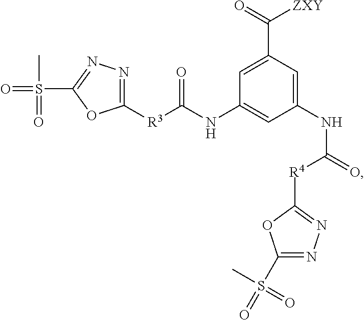

or pharmaceutically acceptable salts thereof, wherein: R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

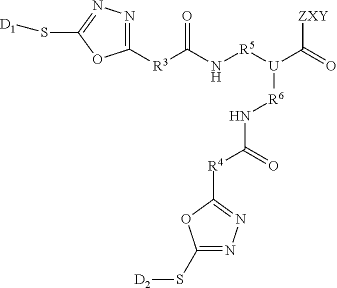

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide, Y is absent or a fatty acid; and D₁ and D₂ independently comprise an amide, amine, imine, sulfonamide, thiourea, urea, thioether, or combinations thereof.

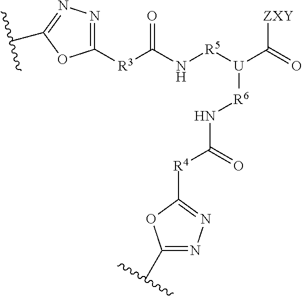

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; U is a tertiary amine or 1,3,5-substituted phenyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

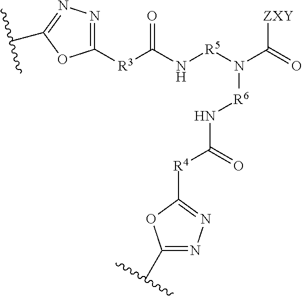

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; R⁵ and R⁶ are independently a covalent bond or C₁ to C₅ alkyl; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

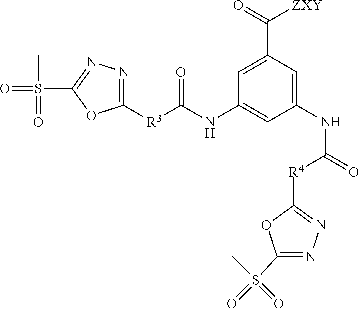

Also disclosed herein are compounds or pharmaceutically acceptable salts thereof of the structure shown above, wherein: R³ and R⁴ are independently a C₁ to C₅ alkyl, which is optionally substituted with one or more of NH₂, CH₂NH₂, and CH₂CH₂NH₂; Z is an O, NH, C₁ to C₃₀ alkyl, (OCH₂CH₂)ₘ, an amino acid polymer, or a combination thereof, wherein m is an integer between 1-30; X is a therapeutic polypeptide; and Y is absent or a fatty acid.

In some embodiments described herein wherein the therapeutic polypeptide is conjugated to Z, Z comprises one or more of: O, C(O), NH, C1 to C30 alkyl, (OCH₂CH₂)ₘ, [C(O)NH—CH₂CH2OCH₂]ₘ, (2-[2-(2-amino-ethoxy)-ethoxy]-acetyl)ₘ, an amino acid, a peptide, or a combination thereof, wherein m is an integer between 1-30. In some embodiments, Z comprises a peptide comprising the sequence of one or more of:

| Peptide | SEQ ID NO: |
|---|---|
| GGGGSGGGGSGGGGS | 1182 |
| GGGGS | 1183 |
| GPAPAPAPAPAPA | 1184 |
| GPAPAPAPAPAPAPAPA | 1185 |
| GKAAAEKAAAEKAAAE | 1186 |
| LEAEAAAKEAAAKEAAAKEAAAKALE | 1187 |
| GGGGSGGGGSGGGGSGGGGS | 1188 |
| GGGGSGGGGSGGGGSGGGGSGGGGS | 1189 |
| PGGGGSGGGGSGGGGS | 1190 |
| SPGGGGSGGGGSGGGGS | 1191 |
| GGGGSGAPAPAPAPAPAP | 1192 |
| GGGGSGGGGSGAPAPAPAPAPAP | 1193 |
| GGGGSGEAAAKEAAAKEAAAK | 1194 |
| GGGGSGGGGSGEAAAKEAAAKEAAAK | 1195 |
| GGGGSGAPAPAPAP | 1196 |
| GGGGSGGGGSGGGGSGAPAPAP | 1197 |
| GGGGSGGGGSGGGGSGGGGSGAPAPAP | 1198 |
| GGGGSGGGGSEAAAKEAAAKEAAAKEAAAK | 1199 |
| GGGGSGGGGSGGGGSGEAAAKEAAAKEAAAK | 1200 |
| GGGGSGGGGSGGGGSGGGGSGEAAAKEAAAKEAAAK | 1201 |
| GGGGSGGGGSGAPAPAPAPAPAPAPAP | 1202 |
| GGGGSGGGGSGGGGSGAPAPAPAPAPAP | 1203 |
| GGGGSGGGGSGGGGSGGGGSGAPAPAPAPAPAP | 1204 |
| GGGGQGGGGQGGGGQ | 1205 |
| GGGGQGGGGQGGGGQGGGGQ | 1206 |
| GGGGQGGGGQGGGGQGGGGQGGGGQ | 1207 |
| GGGGSGGGGSGGGGSGGGGSG | 1208 |
| SGGGGSGGGGSGGGGGPAPAPAPAPAPAG | 1209 |
| SGGGGSGGGGSGGGGGKAAAEKAAAEKAAAEG | 1210 |
| APAPAPAPAPAPGGGGSGGGGS | 1211 |
| GGGGSGGGGSGGGGSGEPEPEPEPEPEP | 1212 |
| GGGGSGEPEPEPEPEPEP | 1213 |
| GGGGQGGGGQGGGGQGEPEPEPEPEPEP | 1214 |
| GPEPEPEPEPEPE | 1215 |
| GPKPKPKPKPKPK | 1216 |
| GEPEPEPEPEPEP | 1217 |

### Linkers

In some embodiments, Formula II comprises a linker, L, to connect a therapeutic polypeptide to an antibody or fragments thereof, such as an Fc region. In certain embodiments, the L of Formula II comprises:

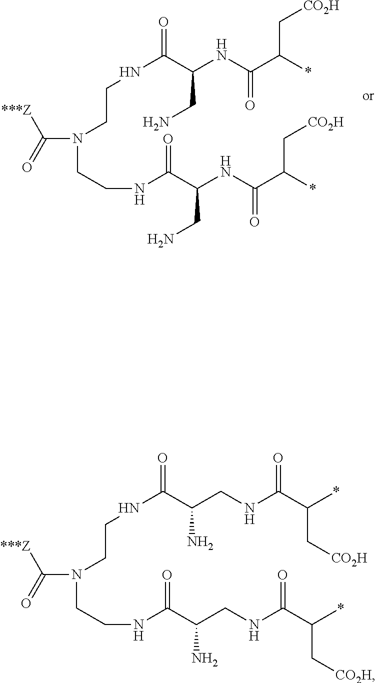

wherein (***) comprises a connection point to the therapeutic polypeptide and (*) comprises a connection point to R⁹ or R¹⁰.

In some other embodiments, the L of Formula II comprises:

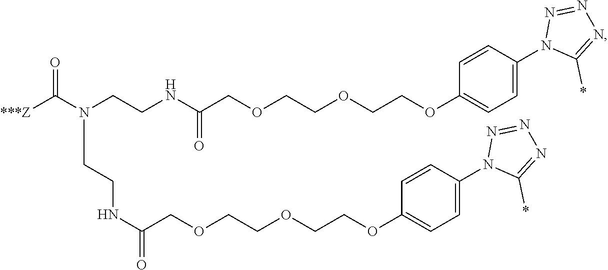

wherein (***) comprises a connection point to the therapeutic polypeptide and (*) comprises a connection point to R⁹ or R¹⁰.

In some embodiments, the present disclosure includes a linker, L, which connects Z via a nucleophilic conjugate addition reaction to another moiety. In some embodiments, the moiety is one or more cysteine amino acids in an Fc region of an antibody or fragment thereof. In some embodiments, the moiety is a sulfur atom from a cysteine residue in an Fc region. In some embodiments, Z via a nucleophilic conjugate addition reaction to another nucleophile, such as an amide, amine, imine, sulfonamide, thiourea, urea, thioether, thiol, cysteine, or combinations thereof, as shown in some of the reactions depicted and described below.

In some embodiments, the linker may include a central trivalent linking unit, U, as shown in Formula IV-A, IV-B, and IV-C above. In some embodiments, a portion of U may be attached to Z. In some embodiments, the other two portions of U comprise a maleimide functional group, e.g., an electrophile, which can be used to selectively connect Z to two nucleophiles in a nucleophilic conjugate addition reaction.

As such, the linker of the present invention comprises two or more maleimide functional groups, preferably two maleimide functional groups, prior to the nucleophilic conjugate addition reaction. Alternatively, the linker of the present invention comprises two or more methyl sulfonyl tetrazole functional groups, or preferably two methyl sulfonyl tetrazole functional groups, prior to the aromatic nucleophilic substitution reaction. Alternatively, the linker of the present invention comprises two or more methyl sulfonyl oxadiazole functional groups, or preferably two methyl sulfonyl oxadiazole functional groups, prior to the aromatic nucleophilic substitution reaction. Alternatively, the linker of the present invention comprises two or more bromo acetyl functional groups, or preferably two bromo acetyl functional groups, prior to the nucleophilic substitution reaction.

Suitable trivalent linking units may include any atom or molecules that can be tri-substituted, such as tri-substituted C₅ to C₈ aryl, tri-substituted C₅ to C₈ heteroaryl with 1 to 3 heteroatoms in the ring system, tri-substituted C₁ to C₅ cycloalkyl, tri-substituted C₅ to C₈ heterocycle with 1 to 3 heteroatoms in the ring system, tertiary amine, tertiary phosphine, or combinations thereof. Preferably, U is a 1,3,5-trisubstituted phenyl or tertiary amine.

In some embodiments, the structures that are attached to U are represented below:

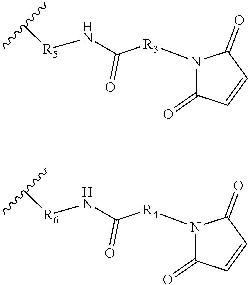

In these structures above, R₃ can be C₁ to C₅ alkyl, optionally substituted with one or more of —NH₂, —CH₂NH₂, and —CH₂CH₂NH₂; and R₅ can be C₁ to C₅ alkyl or absent. R₄ can be C₁ to C₅ alkyl, optionally substituted with one or more of —NH₂, —CH₂NH₂, and —CH₂CH₂NH₂; and R⁶ can be C₁ to C₅ alkyl or absent.

R₃ and/or R₄ can preferably be C₂ alkyl, optionally substituted with one or more of —NH₂, —CH₂NH₂, or —CH₂CH₂NH₂. R₅ and/or R₆ can preferably be C₂ alkyl when U is a tertiary amine and absent when U is 1,3,5-trisubstituted phenyl.

When both structure above are attached to U, R₃ and R₄ can be identical, or they can be independently C₁ to C₅ alkyl, optionally substituted with one or more of —NH₂, —CH₂NH₂, and —CH₂CH₂NH₂. When both structures above are attached to U, R₅ and R₆ can be identical, or they can be independently C₁ to C₅ alkyl or absent. When R₅ and/or R₆ are absent, the amide nitrogen in the structure is directly connected to U, such as when U is 1,3,5-trisubstituted phenyl.

The third arm of the trivalent linking unit attached to U may include Z. Z comprises O, NH, C₁ to C₃₀ alkyl, (OCH₂CH₂)ₘ, an amino acid polymer, or a combination thereof, wherein m is an integer between 1-30; X is a therapeutic polypeptide; Y is absent or a fatty acid; and Fc₁ and Fc₂ are independently an Fc region. Z may be connected directly to U or to a carbonyl functional group. For example, the carbon atom of the carbonyl functional group can be directly attached to U and Z.

Alternatively, each arm that is attached to U and the tetrazole functional group can be represented by the structure below:

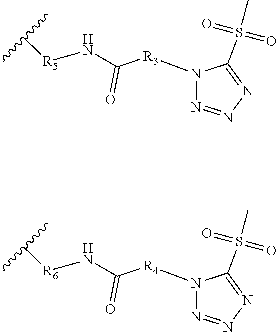

Alternatively, each arm that is attached to U and the oxadiazole functional group can be represented by the formula below:

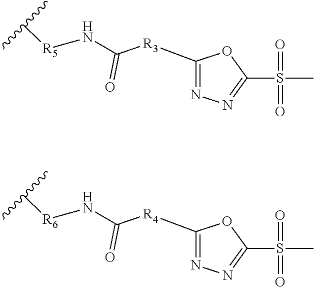

In one embodiment, the first of the two arms, represented by the structures above, R₃ can be phenyl-(OCH₂CH₂)ₘOCH₂ and R₅ can be C₁ to C₅ alkyl or absent. In another embodiment, the second of the two arms, represented by the structures above, R₄ can be phenyl-(OCH₂CH₂)ₘOCH₂ and R₆ can be C₁ to C₅ alkyl or absent.

R₃ and/or R₄ can preferably be phenyl-(OCH₂CH₂)₂OCH₂. R₅ and/or R₆ can preferably be C₂ alkyl when U is a tertiary amine and absent when U is 1,3,5-trisubstituted phenyl.

When both arms are attached to U, R₃ and R₄ can be identical, or they can be independently phenyl-(OCH₂CH₂)₂OCH₂. When both arms are attached to U, R₅ and R₆ can be identical, or they can be independently C₁ to C₅ alkyl or absent. When R₅ and/or R₆ are absent, the amide nitrogen in the structures above is directly connected to U, such as when U is 1,3,5-trisubstituted phenyl.

The third of the three arms attached to U includes Z. Z may comprise H, OH, NH₂, NH, a therapeutic, C₁ to C₃₀ alkyl, (OCH₂CH₂)ₘ, or combinations thereof, and wherein m is a whole number integer from 1 to 30. Z can be connected directly to U or Z can be connected to a carbonyl functional group. The carbon atom of the carbonyl functional group can be directly attached to U and Z.

Alternatively, each arm that is attached to U and the acetyl functional group can be represented by the structures below:

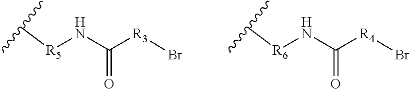

In one embodiment, the first of the two arms, represented by the structures above, R₃ can be C₁ to C₅ alkyl and R₅ can be C₁ to C₅ alkyl or absent. In another embodiments, the second of the two arms, represented by the structures above, R₄ can be C₁ to C₅ alkyl and R₆ can be C₁ to C₅ alkyl or absent.

R₃ and/or R₄ can preferably be C₂ alkyl. R₅ and/or R₆ can preferably be C₂ alkyl when U is a tertiary amine and absent when U is 1,3,5-trisubstituted phenyl.

When both arms are attached to U, R₃ and R₄ can be identical, or they can be independently C₁ to C₅ alkyl. When both arms are attached to U, R⁵ and R⁶ can be identical, or they can be independently C₁ to C₅ alkyl or absent. When R₅ and/or R₆ are absent, the amide nitrogen in Formula V-B or Formula V-C is directly connected to U, such as when U is 1,3,5-trisubstituted phenyl.

The third of the three arms attached to U includes Z. Z can comprise H, OH, NH₂, NH, a therapeutic, C₁ to C₃₀ alkyl, (OCH₂CH₂)ₘ, or combinations thereof, and wherein m is a whole number integer from 1 to 30. Z can be connected directly to U or Z can be connected to a carbonyl functional group. The carbon atom of the carbonyl functional group can be directly attached to U and Z, as shown in Formula II-IV.

Since the reaction conditions for conjugation are relatively mild, the payload, i.e., therapeutic, does not need to be modified to be protected during the reaction. Other linkers disclosed in the art require that therapeutic polypeptide, e.g., insulin, be modified with a recombinant extension. The conjugated compounds disclosed herein are efficacious with a longer duration of action and without the need to modify the therapeutic polypeptide.

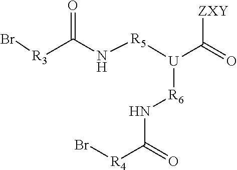

In some embodiments disclosed herein, are compounds of the structure above, and pharmaceutically acceptable salts thereof, wherein R₃ and R₄ are independently selected from C₁ to C₅ alkyl; R₅ and R₆ are independently C₁ to C₅ alkyl or absent; U is tertiary amine or 1,3,5-substituted phenyl; Z comprises O, NH, C₁ to C₃₀ alkyl, (OCH₂CH₂)ₘ, an amino acid polymer, or a combination thereof, X comprises a therapeutic polypeptide; and Y comprises a fatty acid; and wherein: m is an integer between 1-30; and Fc₁ and Fc₂ each comprise an Fc region.

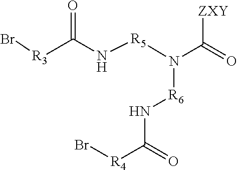

In some embodiments disclosed herein, are compounds of the structure above or pharmaceutically acceptable salts thereof, wherein R₃ and R₄ are independently selected from C₁ to C₅ alkyl; R₅ and R₆ are independently C₁ to C₅ alkyl; Z comprises O, NH, C₁ to C₃₀ alkyl, (OCH₂CH₂)ₘ, an amino acid polymer, or a combination thereof, X comprises a therapeutic polypeptide; and Y comprises a fatty acid; and wherein: m is an integer between 1-30; and Fc₁ and Fc₂ each comprise an Fc region.

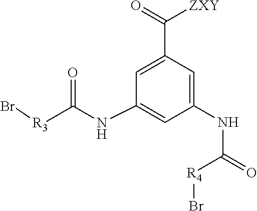

In some embodiments disclosed herein, are compounds of the structure above or pharmaceutically acceptable salts thereof, wherein R₃ and R₄ are independently selected from C₁ to C₅ alkyl; Z comprises O, NH, C₁ to C₃₀ alkyl, (OCH₂CH₂)ₘ, an amino acid polymer, or a combination thereof, X comprises a therapeutic polypeptide; and Y comprises a fatty acid; and wherein: m is an integer between 1-30; and Fc₁ and Fc₂ each comprise an Fc region.

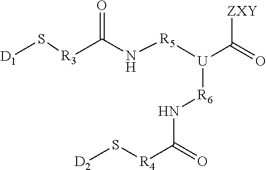

In some embodiments disclosed herein, are compounds of the structure above or pharmaceutically acceptable salts thereof, wherein R₃ and R₄ are independently selected C₁ to C₅ alkyl; R₅ and R₆ are independently C₁ to C₅ alkyl or absent; U is tertiary amine or 1,3,5-substituted phenyl; Z comprises O, NH, C₁ to C₃₀ alkyl, (OCH₂CH₂)ₘ, an amino acid polymer, or a combination thereof, X comprises a therapeutic polypeptide; and Y comprises a fatty acid; and wherein: m is an integer between 1-30; and D₁ and D₂ independently comprise an amide, amine, imine, sulfonamide, thiourea, urea, thioether, or combinations thereof.

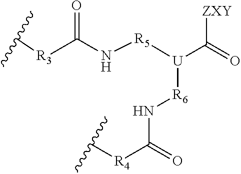

In some embodiments disclosed herein, are compounds of the structure above or pharmaceutically acceptable salts thereof, wherein: R₃ and R₄ are independently selected from C₁ to C₅ alkyl; R⁵ and R⁶ are independently C₁ to C₅ alkyl or absent; U is tertiary amine or 1,3,5-substituted phenyl; Z comprises O, NH, C₁ to C₃₀ alkyl, (OCH₂CH₂)ₘ, an amino acid polymer, or a combination thereof, X comprises a therapeutic polypeptide; and Y comprises a fatty acid; and wherein: m is an integer between 1-30.

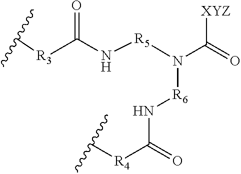

In some embodiments disclosed herein, are compounds of the structure above or pharmaceutically acceptable salts thereof, wherein: R₃ and R₄ are independently selected C₁ to C₅ alkyl; R₅ and R₆ is C₁ to C₅ alkyl; and Z comprises O, NH, C₁ to C₃₀ alkyl, (OCH₂CH₂)ₘ, an amino acid polymer, or a combination thereof, X comprises a therapeutic polypeptide; and Y comprises a fatty acid; and wherein: m is an integer between 1-30.

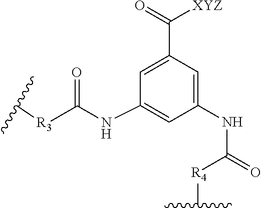

In some embodiments disclosed herein, are compounds of the structure above or pharmaceutically acceptable salts thereof, wherein R₃ and R₄ are independently selected from C₁ to C₅ alkyl; Z comprises O, NH, C₁ to C₃₀ alkyl, (OCH₂CH₂)ₘ, an amino acid polymer, or a combination thereof, X comprises a therapeutic polypeptide; and Y comprises a fatty acid; and wherein: m is an integer between 1-30.

In some embodiments, compounds of present disclosure includes a linker, L, which connects Z via a nucleophilic conjugate addition reaction to another moiety, such as one or more cysteine amino acids in an Fc region of an antibody or fragment thereof, as shown in Formula II, or to another suitable nucleophile, such as an amide, amine, imine, sulfonamide, thiourea, urea, thioether, thiol, cysteine, or combinations thereof, as shown in the structures disclosed herein.

In some embodiments, the linker may include a central trivalent linking unit, U, as shown in the structures disclosed herein. One of the portions of the linker can be attached to Z. The other two portions of the linkers comprise a maleimide functional group, an electrophile, which can be used to selectively connect Z to two nucleophiles in a nucleophilic conjugate addition reaction.

Nucleophilic Conjugate Addition Reactions with Maleimide Functional Groups

As disclosed herein, a linker, L, may be attached to Z, to yield a linked payload, wherein Z further comprises a therapeutic, X, e.g., a linked therapeutic. In some embodiments, the linker, L, comprises two maleimide functional groups. The maleimide functional groups may be used to attach Z to a different moiety, such as an Fc region of an antibody or fragment thereof, through a nucleophilic conjugate addition reaction.

In a nucleophilic conjugate addition reaction, the maleimide is an electrophile and reacts with a nucleophile forming a new covalent bond between the electrophilic carbon atom and a nucleophilic atom, which can lead to an opening of the maleimide functional group, as shown below:

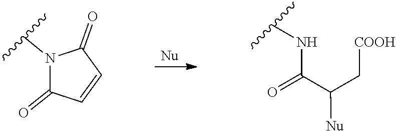

A variety of nucleophiles may be used to react with the electrophilic maleimide functional group. Suitable nucleophiles include moieties comprising one or more nitrogen and/or sulfur atoms in a functional group wherein the nitrogen and/or sulfur atom can serve as an electron-rich nucleophile. Suitable examples of nucleophiles include moieties comprising one or more of the following functional groups: amide, amine, imine, sulfonamide, thiol, thiourea, urea, or combinations thereof, which are shown in the structures below:

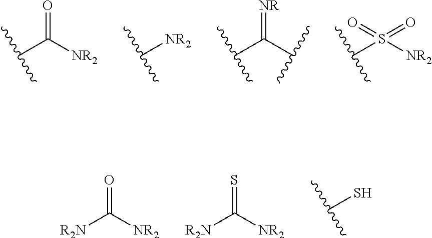

In particular, the amino acid cysteine is found in many peptides, antibodies, proteins, or the like, as shown below, and includes a thiol functional group, which may be used as a nucleophile to form a thioether between a peptide, antibody, protein, or the like and the linker, the linked payload, and/or the linked therapeutic.

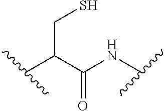 [SMILES: *NC(=O)C(*)CS]

The conditions for nucleophilic conjugate addition reactions are well known to a person of ordinary skill in the art. The conditions may vary according to the selected nucleophiles and electrophiles used for a particular reaction. As disclosed herein, are compounds and/or conjugate compounds comprising a linked payload that has been attached to another moiety through a nucleophilic conjugate addition reaction.

Nucleophilic Aromatic Substitution (SNAr) Reactions with Methylsulfonyl-Tetrazole or Methylsulfonyl-Oxadiazole Functional Groups

As disclosed herein, a linker, L, may be attached to Z, to yield a linked payload, wherein Z further comprises a therapeutic, X, e.g., a linked therapeutic. In some embodiments, the linker, L, comprises two methyl sulfonyl-tetrazole or two methyl sulfonyl-oxadiazole functional groups. The methyl sulfonyl-tetrazole or methyl sulfonyl-oxadiazole functional groups may be used to attach Z to a different moiety, such as an Fc region of an antibody or fragment thereof, through a nucleophilic aromatic substitution reaction.

In a nucleophilic aromatic substitution reaction, the methyl sulfonyl of the tetrazole or the methyl sulfonyl of the oxadiazole acts as a leaving group that is displaced with a nucleophile to form a new covalent bond between the nucleophilic atom and an electrophilic carbon atom as shown below:

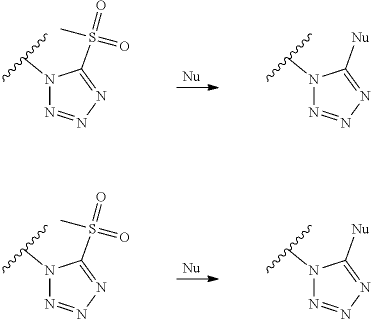

A variety of nucleophiles may be used to react with the electrophilic methyl sulfonyl tetrazole functional group or the methyl sulfonyl oxadiazole functional group. Suitable nucleophiles include moieties comprising one or more nitrogen and/or sulfur atoms in a functional group where the nitrogen and/or sulfur atom can serve as an electron-rich nucleophile. Suitable examples of nucleophiles include moieties comprising of thiol which are also shown below:

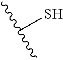 [SMILES: *S]

In particular, the amino acid cysteine is found in many peptides, antibodies, proteins, or the like, as shown below, and includes a thiol functional group, which may be used as a nucleophile to form a thioether between a peptide, antibody, protein, or the like and the linker, the linked payload, and/or the linked therapeutic.

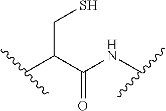 [SMILES: *NC(=O)C(*)CS]

The conditions for aromatic nucleophilic substitution reactions are well known to a person of ordinary skill in the art. The conditions may vary according to the selected nucleophiles and electrophiles used for a particular reaction. As disclosed herein, are compounds and/or conjugate compounds comprising a linked payload that has been attached to another moiety through an aromatic nucleophilic substitution reaction.

Nucleophilic Substitution (S_{N}2) Reactions with Bromo Acetyl Functional Groups

As disclosed herein, a linker L, can be attached to Z, to yield a linked payload, or if Z comprises a therapeutic, a linked therapeutic. The linker, as disclosed herein, comprises two or more, or preferably two, bromo acetyl functional groups, as shown in Formula V-D. The bromo acetyl functional group can be used to attach Z to a different moiety, such as an Fc region, through a nucleophilic substitution reaction.

In the nucleophilic substitution reaction, the bromide of the bromo acetyl acts as a leaving group that is displaced with a nucleophile to form a new covalent bond between the nucleophilic atom and an electrophilic carbon atom as shown below:

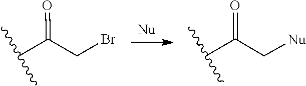

A variety of nucleophiles can be used to react with the electrophilic bromo acetyl functional group. Suitable nucleophiles include moieties comprising one or more nitrogen and/or sulfur atoms in a functional group where the nitrogen and/or sulfur atom can serve as an electron-rich nucleophile. Suitable examples of nucleophiles include moieties comprising one or more of the following functional groups: amide, amine, imine, sulfonamide, thiol, thiourea, urea, or combinations thereof, which are shown in the structures below:

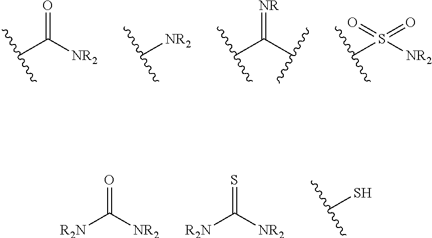

In particular, the amino acid cysteine is found in many peptides, antibodies, proteins, or the like, as shown below, and includes a thiol functional group, which may be used as a nucleophile to form a thioether between a peptide, antibody, protein, or the like and the linker, the linked payload, and/or the linked therapeutic.

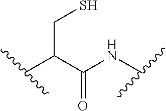 [SMILES: *NC(=O)C(*)CS]

The conditions for nucleophilic substitution reactions are well known to a person of ordinary skill in the art. The conditions may vary according to the selected nucleophiles and electrophiles used for a particular reaction. Disclosed herein, are compounds and/or conjugate compounds comprising a linked payload that has been attached to another moiety through a nucleophilic substitution reaction.

### Duration of Action

The compounds described herein include features that provide extended duration of action for a therapeutic polypeptide. These features include conjugation to an antibody or fragment thereof, such as an Fc region, acylation of the therapeutic polypeptide with an albumin-binding moiety, such as a fatty acid, and/or incorporation of structural features in the therapeutic polypeptide itself that provide for enhanced proteolytic stability. In certain embodiments, compounds that comprise each or all of these features have prolonged duration of action, enabling less frequent administration compared to currently available therapies. For example, in the context of therapeutic polypeptides having binding and activity at one or more of the GIP, GLP-1 and/or glucagon receptors, certain compounds described herein have duration of action that allows for their use in methods of treatment with dosing as infrequent as once-monthly.

Also disclosed herein are methods of increasing the duration of action of a therapeutic. The method can comprise the step of attaching the therapeutic to a trivalent linker to yield a linker therapeutic.

The method may further include the step of conjugating the linked therapeutic to at least a portion of an antibody or fragment thereof, e.g., an Fc region, via a nucleophilic addition or substitution reaction. These reactions occur between the linked therapeutic and two nucleophiles present in the Fc region, such as sulfur atoms and/or anions, resulting in the formation of a conjugate compound.

The resulting conjugate compound comprises a therapeutic molecule covalently attached to the disclosed linkers, which are in turn conjugated to the Fc fragment. The conjugation extends the duration of action beyond that of the unbound therapeutic.

In certain embodiments, the linkers disclosed herein offer notable advantages as the reaction conditions are mild relative to other nucleophilic addition or substitution reactions that have been disclosed with other linkers. For example, the disclosed linkers may be combined with the Fc region of an antibody or fragment thereof with minimal heating. Specifically, the disclosed linkers can be conjugated to the Fc region of an antibody or fragment thereof by mixing the linked therapeutic and a reduced Fc region for no more than 2 hours, at temperatures not exceeding 30° C. or, in certain cases, 25° C.

### Linker Preparations

### Preparation 1

### 4-(Bis(2-((tert-butoxycarbonyl)amino)ethyl)amino)-4-oxobutanoic Acid

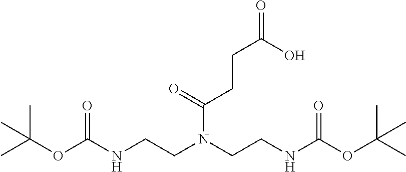 [SMILES: CC(C)(C)OC(=O)NCCN(CCNC(=O)OC(C)(C)C)C(=O)CCC(=O)O]

A solution of succinic anhydride (0.990 g, 9.88 mmol) in DCM (40 mL) was added over a 30-minute period to a solution of 10-oxa-2,5,8-triazadodecanoic acid, 11,11-dimethyl-9-oxo-,1,1-dimethylethyl ester (3.0 g, 9.88 mmol) in DCM (40 mL) at ambient temperature under N₂. The reaction was stirred at ambient temperature for 48 hrs then diluted with H₂O (50 mL) and extracted with DCM (3×40 mL). The organic layers were combined, washed with saturated aqueous NH₄Cl solution, aqueous NaHCO₃, and brine. The organic layer was dried over MgSO₄, filtered, and concentrated in vacuo. The material was absorbed onto silica gel with the aid of DCM, then concentrated under reduced pressure to a free-flowing residue. The residue was purified by normal phase silica gel chromatography and eluted with DCM (5 min) then DCM/MeOH (9:1) to afford the title compound (2.84 g, 70%) as white powder. ES/MS (m/z): 402 (M−).

### Preparation 2

### 1-(4-(5-(Methylsulfonyl)-1H-tetrazol-1-yl)phenoxy)-12-(2-(2-(2-(2-(4-(5-(methylsulfonyl)-1H-tetrazol-1-yl)phenoxy)ethoxy)ethoxy)acetamido)ethyl)-8,13-dioxo-3,6-dioxa-9,12-diazahexadecan-16-oic Acid

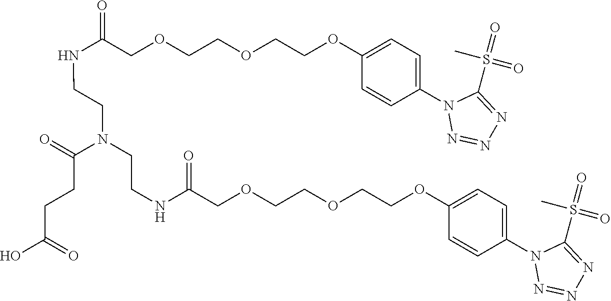 [SMILES: CS(=O)(=O)c1nnnn1-c1ccc(OCCOCCOCC(=O)NCCN(CCNC(=O)COCCOCCOc2ccc(-n3nnnc3S(C)(=O)=O)cc2)C(=O)CCC(=O)O)cc1]

4-(Bis(2-((tert-butoxycarbonyl)amino)ethyl)amino)-4-oxobutanoic acid (0.47 g, 1.05 mmol) was dissolved into DCM (3 mL), then TFA (2 mL) was added. The reaction was stirred at ambient temperature for 3 hrs then concentrated in vacuo. Toluene (10 mL) was added to the residue and the mixture was concentrated in vacuo. This process was repeated once more. The residue was dried under vacuum for two hrs then dissolved into DMF (5 mL). DIEA (0.41 g, 3.15 mmol) was added followed by a solution of 2,5-dioxopyrrolidin-1-yl 2-(2-(2-(4-(5-(methylsulfonyl)-1H-tetrazol-1-yl)phenoxy)ethoxy)ethoxy)acetate (1.07 g, 2.10 mmol) in DMF (4 mL). The reaction was stirred at ambient temperature for 1 hr then diluted with DCM (100 ml), washed with brine followed by H₂O. The organic layer was dried over Na₂SO₄, filtered, and concentrated in vacuo. The residue was purified by normal phase silica gel chromatography. The column was eluted with DCM for 5 minutes then 0% to 20% methanol/DCM over 20 minutes to afford the title compound (0.86 g, 82%) as white solid. ES/MS (m/z): 940 (M+H).

### Preparation 3

### (S)-2-((tert-Butoxycarbonyl)amino)-4-(2,5-dioxo-2,5-dihydro-1H-pyrrol-1-yl)butanoic Acid

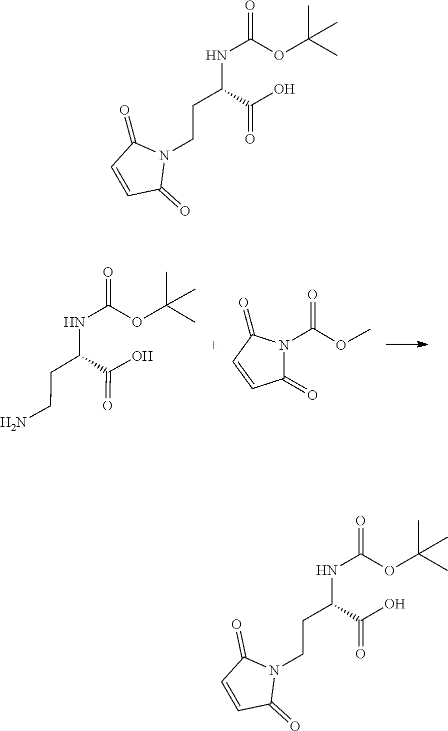 [SMILES: CC(C)(C)OC(=O)N[C@@H](CCN1C(=O)C=CC1=O)C(=O)O ; CC(C)(C)OC(=O)N[C@@H](CCN)C(=O)O ; COC(=O)N1C(=O)C=CC1=O ; CC(C)(C)OC(=O)N[C@@H](CCN1C(=O)C=CC1=O)C(=O)O]

Added triethylamine (1 mL, 7.17 mmol) to a mixture of (2S)-4-amino-2-(tert-butoxycarbonylamino)butanoic acid (500 mg, 2.29 mmol) in 1,4-dioxane (10 mL), THF (5 mL) and water (5 mL). Stirred until a homogenous mixture was obtained, then added methyl 2,5-dioxo-2,5-dihydro-1H-pyrrole-1-carboxylate (370 mg, 2.31 mmol). Mixed at ambient temperature for 1 hour. Diluted with 50 mL of water, adjusted pH to ˜6 by adding 5N HCl. Extracted the aqueous with EtOAc (30 mL) and chloroform/iso-propanol (3×30 mL). Combined the organic phases, dried over sodium sulfate, filtered, and concentrated under reduced pressure. Purified the resulting residue via silica gel column chromatography eluting with 0-30% MeOH in DCM to give the titled compound (580 mg, 81%). ES/MS m/z: 199 (M-tBu).

### Preparation 4

### 2,5-Dioxopyrrolidin-1-yl (S)-2-((tert-butoxycarbonyl)amino)-4-(2,5-dioxo-2,5-dihydro-1H-pyrrol-1-yl)butanoate

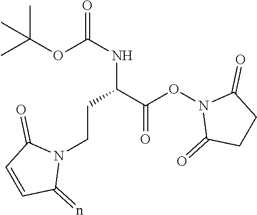

Added dicyclohexylcarbodiimide (320 mg, 1.53 mmol) to a solution of (S)-2-((tert-butoxycarbonyl)amino)-4-(2,5-dioxo-2,5-dihydro-1H-pyrrol-1-yl)butanoic acid (400 mg, 1.27 mmol) and N-hydroxysuccinimide (180 mg, 1.53 mmol) in THF (6 mL). Stirred at ambient temperature for 3 hrs. Filtered to remove solids, then concentrated the filtrate under reduced pressure to provide the title compound (605 mg, 84%) which was used without purification. ES/MS m/z: 296 (M-tBu).

### Preparation 5

### (S)-2-((tert-Butoxycarbonyl)amino)-3-(2,5-dioxo-2,5-dihydro-1H-pyrrol-1-yl)propanoic Acid

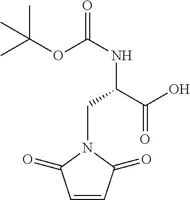 [SMILES: CC(C)(C)OC(=O)N[C@@H](CN1C(=O)C=CC1=O)C(=O)O]

Mixed (2S)-3-amino-2-(tert-butoxycarbonylamino)propanoic acid (5.00 g, 24.5 mmol) with 1M aqueous solution of sodium bicarbonate (130 mL, 130 mmol). Stirred at ambient temperature for 10 min until a clear solution was formed. Cooled the solution in an ice-water bath, and added methyl 2,5-dioxo-2,5-dihydro-1H-pyrrole-1-carboxylate (4.00 g, 25.0 mmol) as in three portions over 15 min. Continued stirring at 0° C. for 3 hrs. Added 100 mL of EtOAc and stirred under cooling, then added concentrated HCl to adjust pH to 1. Separated the layers and extracted the aqueous with DCM (4×50 mL). Combined the organic layers, washed with saturated aqueous NaCl, and dried over sodium sulfate. Removed the solvent under reduced pressure. Purified via silica gel column chromatography eluting with 0-40% MeOH in DCM to give the title compound (5.70 g, 66%). ES/MS m/z: 185 (M-tBu).

### Preparation 6

### 2,5-Dioxopyrrolidin-1-yl (S)-2-((tert-butoxycarbonyl)amino)-3-(2,5-dioxo-2,5-dihydro-1H-pyrrol-1-yl)propanoate

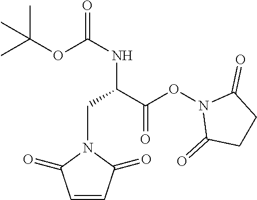 [SMILES: CC(C)(C)OC(=O)N[C@@H](CN1C(=O)C=CC1=O)C(=O)ON1C(=O)CCC1=O]

Added dicyclohexylcarbodiimide (484 mg, 2.32 mmol) to a mixture of (S)-2-((tert-butoxycarbonyl)amino)-3-(2,5-dioxo-2,5-dihydro-1H-pyrrol-1-yl)propanoic acid (550 mg, 1.54 mmol) and N-hydroxysuccinimide (272 mg, 2.31 mmol) in THF (5 mL). Stirred at ambient temperature until a precipitate appeared. Removed the solids by filtration and washed with DCM. Concentrate the filtrate under reduced pressure to give the title compound (1.18 g, 85%). ES/MS m/z: 282 (M+H-Boc).

### Fc Regions

In certain embodiments, the nucleophile capable of reacting with two or more functional groups on the linker is an Fc region, also known as a fragment crystallizable region. The Fc region constitutes the lower constant domain comprised of the full or partial hinge along with the CH2 and CH3 domains or a portion thereof. This Fc fragment may be effector functional, with full engagement to Fc receptors or contain mutations to limit or nullifying Fc receptor binding. Notably, Fc regions of particular antibody classes—such as IgA, IgD, IgE, IgG, IgM, or their combinations—can contain two or more cysteine residues that form a disulfide bridge within the N-terminal portion of the molecule or hinge region.

In some embodiments, the Fc region of an antibody may be modified before its attachment to the linker via a nucleophilic conjugate addition reaction. Such modifications may be designed to minimize unintended reactivity between the antibody and the linker, ensuring that the desired nucleophilic conjugation reaction proceeds efficiently.

In certain embodiments, the Fc region may be a modified version of the antibody's Fc domain, with alterations that include the blunting or removal of specific segments, such as hinge regions.

The disulfide bridge within appropriate Fc regions may be reduced, yielding two thiol groups that serve as nucleophiles capable of undergoing nucleophilic conjugate addition reactions with the maleimide functional groups of the linkers described herein.

Alternatively, the reduced disulfide bridge in suitable Fc regions may provide thiol nucleophiles that can participate in aromatic nucleophilic addition reactions with the methyl sulfonyl tetrazole or methyl sulfonyl oxadiazole functional groups of linkers as described herein.

### Exemplary Fc Regions

The term “Fc region” is used herein to define a C-terminal region of an immunoglobulin heavy chain that contains at least a portion of the constant region. The term includes native sequence Fc regions and variant Fc regions.

As used herein, the term “variant Fc region”, “Fc region variant” or “Fc region mutation” are used interchangeably and each refers to an amino acid sequence of a Fc region that differs from the sequence of a parent Fc region (or fragment thereof) by virtue of at least one amino acid substitution.

As used herein, an “amino acid substitution” refers to the replacement of at least one existing amino acid residue in a given amino acid sequence with another different “replacement” amino acid residue.

Furthermore, substitutions are named herein by the amino acid in the parent Fc followed by the position number at which the substitution occurs followed by the amino acid substituted for the amino acid in the parent Fc region at the same position. For example, the human IgG1 Fc region variant P247I indicates that a proline residue at position 247 of the parent human IgG1 Fc region is substituted by an isoleucine residue).

In one embodiment, a human IgG heavy chain Fc region extends from Cys229 to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

In certain embodiments, the human IgG4 Fc amino acid sequence (hIgG4) is utilized for re-bridging conjugation.

(SEQ ID NO: 935)
AGCPAPEAAGGPSVFLFPPKPKDTLMISRTPEVTCVVVDVSQEDPEVQ
FNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQ
EGNVFSCSVMHEALHNHYTQKSLSLSLG

The Fc region of SEQ ID NO:935 is a native IgG4 with a blunt hinge starting at position 229 (EU index numbering), with an additional N-terminal dipeptide comprised of alanine and glycine (AG). To minimize effector function, the Fc contains 2 mutations, F234A and L235A (EU index numbering) in the CH₂ domain as shown in bold and underlined. Unless otherwise specified, references to “Fc” in the nomenclature for compounds set forth below in Table 3 and described herein, refer to SEQ ID NO:935.

An IgG4 Fc region containing mutations at Q274K, Q355R and E419Q (SEQ ID NO:936) may be used to increase the overall pI of the Fc to help with biophysical properties where a more neutral peptide is used in the conjugation:

(SEQ ID NO: 936)
AGCPAPEAAGGPSVFLFPPKPKDTIMISRTPEVTCVVVDVSQEDPEVK
ENWYVDGVEVHNAKTKPREEQFNSTYRVVSVLIVLHQDWLNGKEYKCK
VSNKGLPSSIEKTISKAKGQPREPQVYTLPPSREEMTKNQVSLTCLVK
GFYPSDIAVEWESNGQPENNYKTTPPVLDSDGSFFLYSRLTVDKSRWQ
QGNVFSCSVMHEALHNHYTQKSLSLSLG

Unless otherwise specified, references to “Fc KRQ” in the nomenclature for compounds set forth below in Table 3 and described herein, refer to SEQ ID NO:936.

To mitigate N-terminal misprocessing in a CHO expression system, several specific signal peptides were identified which provided high titer and minimal N terminal clipping of the first alanine residue:

(SEQ ID NO: 937)
MGWSCIILFLVATATGVHS
(SEQ ID NO: 938)
MDSKVTIICIRFLFWFLLLCMLIGKSHT
(SEQ ID NO: 939)
MKICSLTLLSFLLLAAQVLLVEG
(SEQ ID NO: 940)
MDSKVTIICIRFLFWFLLLCMLIGKSHT

Alternative to changing the signal peptide, 6 alternative residues were explored in place of the alanine on the N-terminus, including Cysteine (C), arginine (R), isoleucine (I), leucine (L), tryosine (Y) and threonine (T), all of which prevented clipping. Alternatively, any of the 20 amino acids except for alanine, serine and glycine which could be used to replace the N terminal residue. Examples include isoleucine N terminal Fc (Fc IG KRQ) and Cysteine N terminal KRQ Fc (Fc N-Cys KRQ), which each include the F234A, L235A, Q274K, Q355R and E419Q mutations as shown in the sequences below.

(SEQ ID NO: 941)
IGCPAPEAAGGPSVFLFPPKPKDTLMISRTPEVTCVVVDVSQEDPEVKF
NWYVDGVEVHNAKTKPREEQFNSTYRVVSVLTVLHQDWLNGKEYKCKVS
NKGLPSSIEKTISKAKGQPREPQVYTLPPSREEMTKNQVSLTCLVKGFY
PSDIAVEWESNGQPENNYKTTPPVLDSDGSFFLYSRLTVDKSRWQQGNV
FSCSVMHEALHNHYTQKSLSLSLG
(SEQ ID NO: 942)
CGCPAPEAAGGPSVFLFPPKPKDTLMISRTPEVTCVVVDVSQEDPEVKF
NWYVDGVEVHNAKTKPREEQFNSTYRVVSVLTVLHQDWLNGKEYKCKVS
NKGLPSSIEKTISKAKGQPREPQVYTLPPSREEMTKNQVSLTCLVKGFY
PSDIAVEWESNGQPENNYKTTPPVLDSDGSFFLYSRLTVDKSRWQQGNV
FSCSVMHEALHNHYTQKSLSLSLG

Other examples include cysteine N terminal Fc (Fc N-Cys), which include the F234A and L235A mutations, as shown in the sequence below.

(SEQ ID NO: 1178)
CGCPAPEAAGGPSVFLFPPKPKDTLMISRTPEVTCVVVDVSQEDPEVQFN
WYVDGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVESCS
VMHEALHNHYTQKSLSLSLG

Unless otherwise specified, references to “Fc IG KRQ” in the nomenclature for compounds set forth below in Table 3 refer to SEQ ID NO:941, references to “Fc N-Cys KRQ” in the nomenclature for compounds set forth below in Table 3 refer to SEQ ID NO:942, and references to “Fc N-Cys” in the nomenclature for compounds set forth below in Table 3 refer to SEQ ID NO:1178.

Beyond the IgG4 subclass, an IgG1 Fc was also explored using an IgG1 with the effector knockout mutations F234A and L235A and P329A (EU index numbering):

(SEQ ID NO: 943)
AGCPAPEAAGGPSVFLFPPKPKDTLMISRTPEVTCVVVDVSHEDPEVKEN
WYVDGVEVHNAKTKPREEQYNSTYRVVSVLIVLHQDWINGKEYKCKVSNK
ALAAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPGK

In some embodiments, a wild type IgG1 Fc with full effector function may be utilized in place of the effector knockout version:

(SEQ ID NO: 944)
AGCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPGK

As an alternative to the re-bridging approach described above, engineered cysteines were introduced at positions 358, 398 and 415 of an IgG4 Fc with a full hinge starting at position 216 and containing the S228P mutation to enhance hinge stability. The Fc fragment contains 2 engineered cysteines and results in a symmetrical molecule with a peptide on each half or 2 per Fc conjugate.

eCys358 Fc:
(SEQ ID NO: 1179)
ESKYGPPCPPCPAPEAAGGPSVFLFPPKPKDTLMISRTPEVTCVVVDVSQEDPEVKENWYVDG
VEVHNAKTKPREEQFNSTYRVVSVLTVLHQDWLNGKEYKCKVSNKGLPSSIEKTISKAKGQPR
EPQVYTLPPSQEECTKNQVSLTCLVKGFYPSDIAVEWESNGQPENNYKTTPPVLDSDGSFFLY
SRLTVDKSRWQEGNVFSCSVMHEALHNHYTQKSLSLSLG
eCys398 Fc:
(SEQ ID NO: 1180)
ESKYGPPCPPCPAPEAAGGPSVFLFPPKPKDTLMISRTPEVTCVVVDVSQEDPEVKENWYVDG
VEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVCDSDGSFFLY
SRLTVDKSRWQEGNVFSCSVMHEALHNHYTQKSLSLSLG
eCys415 Fc:
(SEQ ID NO: 1181)
ESKYGPPCPPCPAPEAAGGPSVFLFPPKPKDTLMISRTPEVTCVVVDVSQEDPEVKENWYVDG
VEVHNAKTKPREEQFNSTYRVVSVLIVLHQDWINGKEYKCKVSNKGLPSSIEKTISKAKGQPR
EPQVYTLPPSQEEMTKNQVSLTCLVKGFYPSDIAVEWESNGQPENNYKTTPPVLDSDGSFFLY
SRLTVDKCRWQEGNVFSCSVMHEALHNHYTQKSLSLSLG

To generate an Fc with a single peptide conjugated through engineered cysteines, an Fc with a full hinge starting at position 216, lacking any eCys site was generated with mutation F405L and R409K in the CH3 domain. An Fc exchange approach was utilized to pair a half Fc lacking an eCys site with a half Fc containing the desired eCys site, followed by conjugation.

(SEQ ID NO: 948)
ESKYGPPCPPCPAPEAAGGPSVFLFPPKPKDTLMISRTPEVTCVVVDVSQ
EDPEVKENWYVDGVEVHNAKTKPREEQFNSTYRVVSVLTVLHQDWLNGKE
YKCKVSNKGLPSSIEKTISKAKGQPREPQVYTLPPSREEMTKNQVSLTCL
VKGFYPSDIAVEWESNGQPENNYKTTPPVLDSDGSELLYSKLTVDKSRWQ
QGNVFSCSVMHEALHNHYTQKSLSLSLG

In certain embodiments, compounds of the present disclosure comprise an Fc region having at least 90% sequence identity, or at least 95% sequence identity, or at least 97% sequence identity, or at least 98% sequence identity, or at least 99% sequence identity to any of SEQ ID NOs. SEQ ID NOs:935-936, 941-948; 1178-1181.

### General Methodologies and Conditions for Addition of Fcs

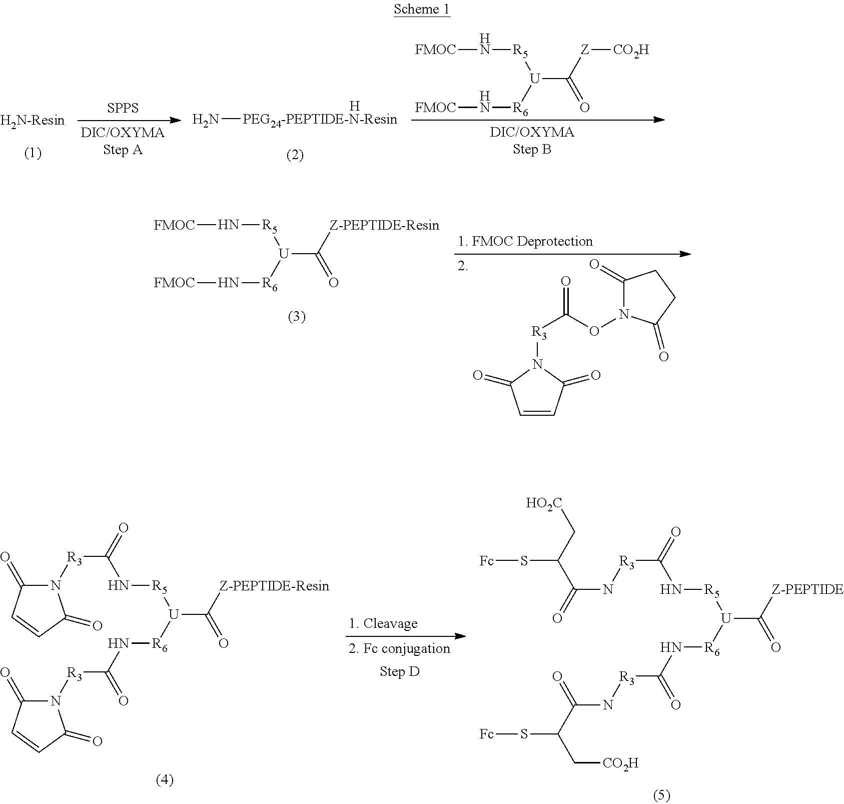

Scheme 1, Step A depicts the synthesis of the H₂N-PEG₂₄-peptide generated by solid-phase peptide synthesis using Fmoc/t-Bu strategy on a Classic Symphony automated peptide synthesizer starting from RAPP AM-Rink Amide resin to afford compound (2). Step B shows the reaction of the PEGylated peptide (2) with the bis-Fmoc protected trivalent core where U is either 1,3,5-trisubstituted phenyl or tertiary amine (Preparation 1) to provide compound (3). In Step C the Fmoc groups were removed, and the resultant primary amines were reacted with NHS esters MalDap (commercially available), MalDab (Preparation 15), or Beta-MalDap (Preparation 17) to afford compound (4). In Step D, the peptide was cleaved from the resin. The bismaleimide functional groups were reacted with two reduced thiol functional groups in an Fc region in a nucleophilic conjugate addition reaction to yield the conjugate (5).

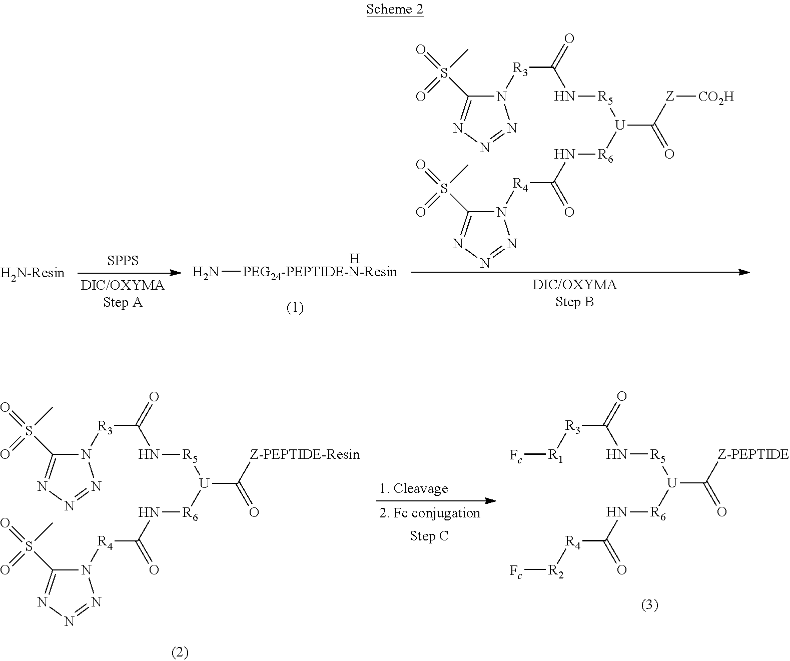

Scheme 2, Step A depicts the synthesis of the H₂N-PEG₂₄-peptide generated by solid-phase peptide synthesis to afford compound (1). Step B shows the coupling of the PEGylated peptide (21) with the bis-(methyl sulfonyl)tetrazole analog (Preparation 12) to provide compound (2). In Step C, the peptide was cleaved from the resin. The bis-(methyl sulfonyl)tetrazole functional groups were reacted with two reduced thiol functional groups in an Fc region in a nucleophilic conjugate addition reaction to yield the conjugate (3).

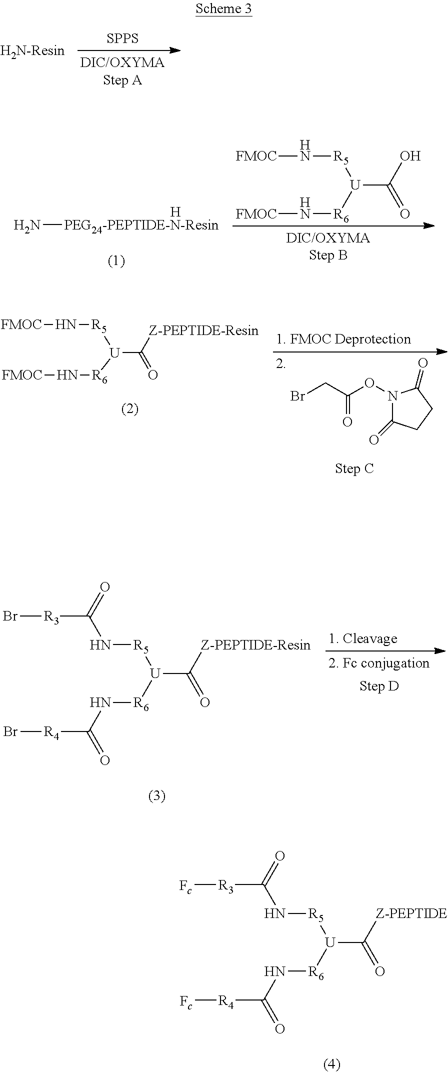

Scheme 3, Step A depicts the synthesis of the H₂N-PEG₂₄-peptide generated by solid-phase peptide synthesis to afford compound (1). Step B shows the coupling of the PEGylated peptide (2) with the bis-Fmoc protected trivalent core where U is either 1,3,5-trisubstituted phenyl or tertiary amine (Preparation 1) to provide compound (2). In Step C the Fmoc groups were removed, and the resultant primary amines were reacted with 2,5-dioxopyrrolidin-1-yl 2-bromoacetate to provide compound (3). The functional groups of compound (3) were reacted with two reduced thiol functional groups in an Fc region in a nucleophilic conjugate addition reaction to yield the conjugate (4).

### Therapeutic Polypeptides

The term “therapeutic polypeptide” refers to any amino acid polymer having activity suitable for use in treatment and/or prevention of a disease or condition. The conjugate structures herein may be used to provide extended duration of action for a variety of therapeutic polypeptides. A non-limiting list of examples of such therapeutics includes the following, or variants thereof. GIP, GLP-1: GLP-2; GIP; oxyntomodulin (OXM); Amylin; Apelin; Urocortin (UCN), ANP; BNP; CNP; parathyroid hormone (PTH); MC4 agonist; Somatostatin; GDF15; peptide YY (PYY); Pancreatic poly peptide; ACTH; VIP; Oxytocin; Grehlin; Gastrin; Insulin; human growth hormone (hGH): fibroblast grown factor 21 (FGF21); interleukin 2 (IL2); IL10; IL15; IL22; IL7; Pepstatin; Calcitonin; erythropoietin (EPO); thrombopoietin and receptor agonists; Angiotensin; Vasopressin; Thymosin beta-4; Guanylate cyclase-C agonists: epidermal growth factor (EGF): alpha-1-anti-trypsin: corticotropin releasing hormone (CRH); GnRH; Thymosin alpha 1; follicle stimulating hormone (FSH); thyroid stimulating hormone (TSH); thyroid releasing hormone (TRH); melanocortin stimulating hormone (MSH): apo-CII mimetic peptide; Factor IX: Factor VIII: Factor X; Granulocyte colony-stimulating factor: Interferon-alpha; beta-amyloid inhibitors; CD40 ligand: CD137 agonist; CD24: CTLA4: TNFR; ENPP1; SIRP-alpha; and Ulinastatin.

In certain embodiments, the therapeutic polypeptide comprises an agonist at one or more of the GIP, GLP-1 and glucagon receptors. GIP is a 42-amino acid peptide and is an incretin which plays a physiological role in glucose homeostasis by stimulating insulin secretion from pancreatic beta cells in the presence of glucose. GLP-1 is a 36-amino acid peptide and is an incretin which stimulates glucose-dependent insulin secretion, and which has been shown to prevent hyperglycemia in diabetics. The major biologically active fragment of GLP-1 is produced as a 30-amino acid, C-terminal amidated peptide (GLP-1₇₋₃₆). Glucagon is a 29-amino acid peptide that helps maintain blood glucose by binding to and activating glucagon receptors on hepatocytes, causing the liver to release glucose, stored in the form of glycogen, through a process called glycogenolysis.

In certain embodiments, exemplary compounds of the present disclosure comprise a dual agonist at the GIP and GLP-1 receptors. In some other embodiments, exemplary compounds of the present disclosure comprise a triple agonist at the GIP, GLP-1 and glucagon receptors.

Compounds of the present disclosure having activity at one or more of the GIP, GLP-1 and/or glucagon receptors have potential for therapeutic value in a number of conditions, diseases or disorders, including, but not limited to, T2DM, obesity, dyslipidemia; cardiovascular disease (CVD); major adverse cardiovascular events (MACE); chronic kidney disease (CKD); heart failure (HF); hypertension (HT); peripheral arterial disease (PAD); obstructive sleep apnea (OSA); metabolic syndrome; type 2 diabetes mellitus (T2DM); glycemic control; osteoarthritis (OA); chronic lower back pain (CLBP); neurodegenerative and/or cognitive disorders such as Alzheimer's disease (AD); Parkinson's disease; metabolic steatohepatitis (MASH): hepatic cirrhosis; fatty liver disease (FLD); polycystic ovary syndrome and/or alcohol abuse disorder, NAFLD and NASH, metabolic syndrome, bone-related disorders, See. e.g., Jall et al. (2017) Mol. Metab. 6:440-446; Carbone et al. (2016) J. Gastroenterol. Hepatol. 31:23-31; Finan et al. (2016) Trends Mol. Med. 22:359-376; Choi et al. (2017) Potent body weight loss and efficacy in a NASH animal model by a novel long-acting GLP-1/Glucagon/GIP triple-agonist (HM15211), ADA Poster 1139-P; Ding (2008) J. Bone Miner. Res. 23:536-543; Tai et al. (2018) Brain Res. 1678:64-74; Müller et al. (2017) Physiol. Rev. 97:721-766; Finan et al. (2013) Sci. Transl. Med. 5:209; Hölschcr (2014) Biochem. Soc. Trans. 42:593-600.

In certain embodiments, the therapeutic polypeptide comprises:

(SEQ ID NO: 1218)
X₁X₂X₃X₄TX₆TSDX₁₀X₁₁X₁₂X₁₃LX₁₅X₁₆KAX₁₉X₂₀X₂₁FIX₂₄X₂₅LX₂₇X₂₈X₂₉
X₃₀X₃₁X₃₂X₃₃X₃₄X₃₅X₃₆X₃₇X₃₈X₃₉X₄₀X₄₁,

wherein:
X₁ is H, NMeY, or Y;
X₂ is Ac4c, Aib, αMeS, Iva, or D-Ala;
X₃ is Q, E, or H;
X₄ is G or D-Ala;
X₆ is αMeF(2F), F, or αMeF;
X₁₀ is 4-Pal, Y, or V;
X₁₁ is Aib, S, or αMeS;
X₁₂ is I or S;
X₁₃ is L or αMeL;
X₁₅ is D or E;
X₁₆ is K, E or Orn;
X₁₉ is A or Q
X₂₀ is Aib, αMeL, Iva, or αMe4Pal;
X₂₁ is E, Q, D, or Orn;
X₂₄ is K, Q, E, D-Glu, or D-Gln;
X₂₅ is αMeY, W, or Y;
X₂₇ is I, L, or V;
X₂₈ K, E or A;
X₂₉ is Aib, G, or Q;
X₃₀ is G or S;
X₃₁ is P, G, E, or Orn;
X₃₂ is absent, S or P;
wherein if X₃₂ is S or P, then X₃₃ is S;
wherein if X₃₃ is S, then is X₃₄ is G or Aib;
wherein if X₃₄ is G or Aib, then X₃₅ is absent or A or Orn;
wherein if X₃₅ is A or Orn, then X₃₆ is absent or P;
wherein if X₃₆ is P, then X₃₇ is absent or P;
wherein if X₃₇ is P, then X₃₈ is absent or P;
wherein if X₃₈ is P, then X₃₉ is absent or S, Orn, or G;
wherein if X₃₉ is S, Orn, or G, then X₄₀ is absent, K, or G;
wherein if X₄₀ is K or G, then X₄₁ is absent or S or G;
wherein if X₃₂ is absent, then X₃₃ through X₄₁ are also absent;
wherein if X₃₅ is absent, then X₃₆ through X₄₁ are also absent;
wherein if X₃₆ is absent, then X₃₇ through X₄₁ are also absent;
wherein if X₃₇ is absent, then X₃₈ through X₄₁ are also absent;
wherein if X₃₈ is absent, then X₃₉ through X₄₁ are also absent;
wherein if X₃₉ is absent, then X₄₁ and X₄₁ are also absent;
wherein if X₄₀ is absent, then X₄₁ is absent.

In certain embodiments, the therapeutic polypeptide comprises specific features that result in optimization of properties such as binding and/or activity at the GIP, GLP-1 and/or glucagon receptors, increasing proteolytic stability, improving developability and/or optimization of pharmacokinetics and pharmacodynamics. In certain embodiments, the therapeutic polypeptide comprises one or more of X₆ is αMeF(2F); X₁₀ is Y; X₁₁ is Aib or αMeS; X₁₂ is I; X₁₃ is αMeL; X₁₅ is D; X₁₆ is Orn; X₂₀ is Aib; X₂₁ is E or Q; X₂₄ is E; X₂₅ is αMeY; X₂₇ is I; X₂₈ is K or E; X₂₉ is Aib or G; X₃₀ is S; X₃₁ is P or G; X₃₂ is absent or S; X₃₂—X₃₉ is SSGAPPPS; and X₄₀ is K. In certain embodiments, In certain embodiments, the therapeutic polypeptide comprises each of X₁₀ is Y; X₂₄ is E; X₃₀ is S; and X₄₀ is K.

In certain embodiments, the therapeutic polypeptide comprises a 90% sequence identity to any of SEQ ID NO:1 to SEQ ID NO:186 or SEQ ID NO:996 to SEQ ID NO:1038. In certain embodiments, the therapeutic polypeptide comprises any of SEQ ID NO:1 to SEQ ID NO:186 or SEQ ID NO:996 to SEQ ID NO:1038.

In certain embodiments, the therapeutic polypeptide comprises:

(SEQ ID NO: 1219)
YAibEGTX₆TSDX₁₀X₁₁X₁₂X₁₃LX₁₅X₁₆KAQX₂₀X₂₁FIX₂₄X₂₅LX₂₇X₂₈X₂₉
X₃₀X₃₁X₃₂X₃₃X₃₄X₃₅X₃₆X₃₇X₃₈X₃₉X₄₀X₄₁,

wherein:
X₆ is αMeF(2F), F, or αMeF;
X₁₀ is 4-Pal, Y, or V;
X₁₁ is Aib or S;
X₁₂ is I or S;
X₁₃ is L or αMeL;
X₁₅ is D or E;
X₁₆ is E or Orn;
X₂₀ is Aib, αMeL, or Iva;
X₂₁ is E, Q, D, or Orn;
X₂₄ is K, Q, E, or D-Glu;
X₂₅ is αMeY or Y;
X₂₇ is I, L, or V;
X₂₈ K, E or A;
X₂₉ is Aib, G, or Q;
X₃₀ is G or S;
X₃₁ is P, G, E, or Orn;
X₃₂ is absent, S, or P;
wherein if X₃₂ is S or P, then X₃₃ is S:
wherein if X₃₃ is S, then is X₃₄ is G or Aib;
wherein if X₃₄ is G or Aib, then X₃₅ is absent, A, or Orn;
wherein if X₃₅ is A or Orn, then X₃₆ is absent or P;
wherein if X₃₆ is P, then X₃₇ is absent or P;
wherein if X₃₇ is P, then X₃₈ is absent or P;
wherein if X₃₈ is P, then X₃₉ is absent, S, Orn, or G;
wherein if X₃₉ is S, Orn, or G, then X₄₀ is absent, K, or G;
wherein if X₄₀ is K or G, then X₄₁ is absent, S, or G;
wherein if X₃₂ is absent, then X₃₃ through X₄₁ are also absent;
wherein if X₃₅ is absent, then X₃₆ through X₄₁ are also absent;
wherein if X₃₆ is absent, then X₃₇ through X₄₁ are also absent;
wherein if X₃₇ is absent, then X₃₈ through X₄₁ are also absent;
wherein if X₃₈ is absent, then X₃₉ through X₄₁ are also absent;
wherein if X₃₉ is absent, then X₄₀ and X₄₁ are also absent;
wherein if X₄₀ is absent, then X₄₁ is absent.

In some embodiments, the polypeptide of SEQ ID NO: 1219 has dual agonist activity at the GIP and GLP-1 receptors. In some embodiments, the polypeptide comprises at least one of: X₁₀ is Y; X₁₁ is Aib; X₂₄ is E and X₃₀ is S. In some embodiments, the polypeptide comprises at least two of: X₁₀ is Y; X₁₁ is Aib; X₂₄ is E and X₃₀ is S. In some embodiments, the polypeptide comprises each of: X₁₀ is Y; X₁₁ is Aib; X₂₄ is E and X₃₀ is S.

In some embodiments, the polypeptide further comprises at least one of X₆ is αMeF(2F); X₁₂ is I; X₁₃ is αMeL; X₁₅ is D; X₁₆ is Orn; X₂₀ is Aib; X₂₁ is E or Q; X₂₅ is αMeY; X₂₇ is 1; X₂₈ is K or E; X₂₉ is Aib or G; X₃₁ is P or G; X₃₂ is absent or S; and X₄₀ is K. In some embodiments, the polypeptide comprises each of X₆ is αMeF(2F); X₁₂ is I; X₁₃ is αMeL; X₁₅ is D; X₁₆ is Orn; X₂₀ is Aib; X₂₁ is E or Q; X₂₅ is αMeY; X₂₇ is I; X₂₈ is K or E; X₂₉ is Aib or G; X₃₁ is P or G; X₃₂ is absent or S; and X₄₀ is K. In some embodiments, X₂₁ is E; X₂₈ is E; X₂₉ is G; X₃₁ is P, and X₃₂ is S.

In certain embodiments, the therapeutic polypeptide comprises each of X₁ is Y; X₂ is Aib; X₃ is E; X₄ is G; X₆ is αMeF(2F); X₁₀ is 4-Pal or Y; X₁₁ is Aib or S; X₁₂ is I; X₁₃ is αMeL; X₁₅ is D; X₁₆ is E or Orn; X₁ is Q; X₂₀ is Aib; X₂₁ is E; X₂₄ is Q, E, or D-Glu; X₂₃ is αMeY; X₂₇ is I; X₂₈ E; X₂₉ is Aib or G; X₃₀ is G or S; X₃₁ is P; X₃₂ is G or S; wherein if X₃₂ is S then X₃₃ is S; X₃₄ is G; X₃₅ is A; X₃₆ is P; X₃₇ is P; X₃₈ is P; X₃₉ is S; X₄₀ is K; and X₄₁ is absent; wherein if X₃₂ is G, then X₃₃ through X₄₁ are absent. In certain embodiments, the therapeutic polypeptide comprises at least one of: X₁₀ is Y or 4-Pal; X₁₁ is Aib or S; X₂₄ is Q, E, or d-Glu, and X₃₀ is G or S. In certain embodiments, the therapeutic polypeptide comprises each of: X₁₀ is Y or 4-Pal; X₁₁ is Aib; X₂₄ is E; X₃₀ is S; and X₄₀ is K. In some embodiments, one or more of the amino acid modifications described above result in the therapeutic polypeptide having improved proteolytic stability. In some embodiments, one or more of the amino acid modifications described above result in the therapeutic polypeptide having increased activity at the GIP and/or GLP-1 receptors.

In some embodiments, the therapeutic polypeptide comprises a 90% sequence identity to any of SEQ ID NO:1 to SEQ ID NO:92 or SEQ ID NO:996 to SEQ ID NO:999. In some embodiments, the therapeutic polypeptide comprises one or more conservative amino acid substitutions to any of SEQ ID NO:1 to SEQ ID NO:92 or SEQ ID NO:996 to SEQ ID NO:999. In some embodiments, the therapeutic polypeptide comprises no more than 5, no more than 4, no more than 3, no more than 2 or no more than 1 conservative amino acid substitutions to any of SEQ ID NO:1 to SEQ ID NO:92 or SEQ ID NO:996 to SEQ ID NO:999. In some embodiments, the therapeutic polypeptide comprises any of SEQ ID NO:1 to SEQ ID NO:92 or SEQ ID NO:996 to SEQ ID NO:999. In some embodiments, the therapeutic polypeptide comprises any of SEQ ID NOs: 1, 2, 22, 43, 45, 48, 55, 56, 58, 59, 61, 66, 68 and 47.

In other embodiments, the therapeutic polypeptide comprises:

(SEQ ID NO: 1220)
X₁X₂X₃X₄TX₆TSDX₁₀X₁₁X₁₂X₁₃LX₁₅X₁₆KAQX₂₀X₂₁FIX₂₄X₂₅LX₂₇X₂₈X₂₉
X₃₀X₃₁X₃₂X₃₃X₃₄X₃₅X₃₆X₃₇X₃₈X₃₉X₄₀X₄₁,

wherein:
X₁ is H, NMeY, or Y;
X₂ is Ac4c, Aib, αMeS, Iva, or D-Ala;
X₃ is Q, E, or H;
X₄ is G or D-Ala;
X₆ is αMeF(2F), F, or αMeF;
X₁₀ is 4-Pal, Y, or V;
X₁₁ is Aib, S, or MeS;
X₁₂ is I or S;
X₁₃ is L or αMeL;
X₁₅ is D or E;
X₁₆ is E or Orn;
X₂₀ is Aib, αMeL, Iva, or αMe4Pal;
X₂₁ is E, Q, D, or Orn;
X₂₄ is K, Q, E, D-Glu, or D-Gln;
X₂₅ is αMeY or Y;
X₂₇ is I, L, or V;
X₂₈ K, E or A;
X₂₉ is Aib, G, or Q;
X₃₀ is G or S;
X₃₁ is P, G, E, or Orn;
X₃₂ is absent, S or P;
wherein if X₃₂ is S or P, then X₃₃ is S;
wherein if X₃₃ is S, then is X₃₄ is G or Aib;
wherein if X₃₄ is G or Aib, then X₃₅ is absent or A or Orn;
wherein if X₃₅ is A or Orn, then X₃₆ is absent or P;
wherein if X₃₆ is P, then X₃₇ is absent or P;
wherein if X₃₇ is P, then X₃₈ is absent or P;
wherein if X₃₈ is P, then X₃₉ is absent or S, Orn, or G;
wherein if X₃₉ is S, Orn, or G, then X₄₁ is absent, K, or G;
wherein if X₄₀ is K or G, then X₄₁ is absent or S or G;
wherein if X₃₂ is absent, then X₃₃ through X₄₁ are also absent;
wherein if X₃₅ is absent, then X₃₆ through X₄₁ are also absent;
wherein if X₃₆ is absent, then X₃₇ through X₄₁ are also absent;
wherein if X₃₇ is absent, then X₃₈ through X₄₁ are also absent;
wherein if X₃₈ is absent, then X₃₉ through X₄₁ are also absent;
wherein if X₃₉ is absent, then X₄₀ and X₄₁ are also absent;
wherein if X₄₀ is absent, then X₄₁ is absent.

In certain embodiments, the therapeutic polypeptide of SEQ ID NO: 1220 has tri-agonist activity at the GIP, GLP-1 and glucagon receptors. In some embodiments, the therapeutic polypeptide comprises at least one of: X₁₀ is Y, X₂₄ is E, X₃₀ is S and X₄₀ is K. In some embodiments, the therapeutic polypeptide comprises each of: X₁₀ is Y, X₂₄ is E, X₃₀ is S and X₄₀ is K.

In some embodiments, the therapeutic polypeptide comprises at least one of: X₁ is H or Y; X₂ is Ac4c or Aib; X₃ is Q; X₄ is G; X₆ is αMeF(2F); X₁₁ is αMeS; X₁₂ is I; X₁₃ is αMeL; X₁₅ is D; X₁₆ is Orn; X₂₀ is αMe4Pal; X₂₁ is E or Orn; X₂₃ is αMeY; X₂₇ is I; X₂₈ E; X₂₉ is Aib or G; X₃₁ is P; X₃₂ is S; X₃₃ is S; X₃₄ is G; X₃₅ is A; X₃₆ is P; X₃₇ is P; X₃₈ is P; X₃₉ is S. In some embodiments, the therapeutic polypeptide comprises each of: X₁ is H or Y; X₂ is Ac4c or Aib; X₃ is Q; X₄ is G; X₆ is αMeF(2F); X₁₁ is αMeS; X₁₁ is 1; X₁₃ is αMeL; X₁₅ is D; X₁₆ is Orn; X₂₀ is αMe4Pal; X₂₁ is E or Orn; X₂₅ is αMeY; X₂₇ is I; X₂₈ E; X₂₉ is Aib or G; X₃₁ is P; X₃₂ is S; X₃₃ is S; X₃₄ is G; X₃₅ is A; X₃₆ is P; X₃₇ is P; X₃₈ is P; X₃₉ is S. In some embodiments, X₁ is Y; X₂ is Aib; X₃ is Q; X₂₁ is E; X₂₉ is G.

In some embodiments, the therapeutic polypeptide comprises each of: X₁ is H or Y; X₂ is Aib; X₃ is Q; X₄ is G; X₆ is αMeF(2F); X₁₀ is Y or 4-Pal; X₁₁ is S or αMeS; X₁₂ is I; X₁₃ is αMeL; X₁₅ is D; X₂₀ is Aib or αMe4Pal; X₂₁ is E or Orn; X₂₄ is E or d-Glu; X₂₅ is αMeY; X₂₇ is L or I; X₂₈ is E; X₂₉—X₃₉ is GSPSSGAPPPS; and X₄₀ is K. In some embodiments, the therapeutic polypeptide comprises at least one of: X₁₀ is Y or 4-Pal; X₂₄ is E or d-Glu, and X₃₀ is S. In some embodiments, the therapeutic polypeptide comprises each of: X₁₀ is Y or 4-Pal; X₂₄ is E or d-Glu, X₃₀ is S and X₄₀ is K and is conjugated to the linker.

In some embodiments, one or more of the amino acid modifications described above result in the therapeutic polypeptide having improved proteolytic stability. In some embodiments, one or more of the amino acid modifications described above result in the therapeutic polypeptide having increased activity at the GIP and/or GLP-1 receptors.

In some embodiments, the therapeutic polypeptide comprises a 90% sequence identity to any of SEQ ID NOs: 93-186 or SEQ ID NOs:1000-1038. In some embodiments, the therapeutic polypeptide comprises one or more conservative amino acid substitutions to any of SEQ ID NOs: 93-186 or SEQ ID NOs:1000-1038. In some embodiments, the therapeutic polypeptide comprises no more than 5, no more than 4, no more than 3, no more than 2 or no more than 1 conservative amino acid substitutions to any of SEQ ID NOs: 93-186 or SEQ ID NOs:1000-1038. In some embodiments, the therapeutic polypeptide comprises any of SEQ ID NOs: 93-186 or SEQ ID NOs:1000-1038. In some embodiments, the therapeutic polypeptide comprises any of SEQ ID NOs: 97, 113, 128, 130, 144, 171, 183.

The structural features of the compounds described in certain embodiments herein result in them having appropriate activity at each of the GIP, GLP-1 and glucagon receptors to obtain the favorable effects of activity at each receptor (i.e., triple agonist activity), but not so much activity at any one receptor to either overwhelm the activity at the other two receptors or result in undesirable side effects when administered at a dose sufficient to result in activity at all three receptors.

The affinity of the polypeptides described herein for each of the GIP, GLP-1 and glucagon receptors may be measured using techniques known in the art for measuring receptor binding levels and is commonly expressed as an inhibitory constant (Ki) value. The activity of the polypeptides described herein at each of the receptors also may be measured using techniques known in the art, including, for example, the in vitro activity assays described below, and is commonly expressed as an effective concentration 50 (EC₅₀) value, which is the concentration of compound causing half-maximal simulation in a dose response curve.

In some embodiments, the polypeptides described herein are partial agonists at the GLP-1 receptor showing agonism of 80% or less compared to the native GLP-1₇₋₃₆ (SEQ ID NO:1221) as demonstrated by the HEK293 cell GLP-1 receptor internalization assay described herein. In other embodiments, the polypeptides described herein are full agonists at the GLP-1 receptor showing agonism of ≥80% compared to the native GLP-1₇₋₃₆ (SEQ ID NO:1221) as demonstrated by the HEK293 cell GLP-1 receptor internalization assay described herein. In some embodiments, the polypeptides described herein have greater potency at one or more of the glucagon, GIP and GLP-1 receptors as compared to native glucagon (SEQ ID NO:1222), GIP (SEQ ID NO:1223) and GLP-1₇₋₃₆ (SEQ ID NO:1221).

In addition to the sequences described herein, the polypeptides described herein may include one or more conservative amino acid substitutions, provided, however, that the polypeptides remain capable of binding to and activating GIP, GLP-1 and/or glucagon receptors.

The structural features of certain embodiments of compounds described herein also result in the compounds having many other beneficial attributes relevant to their developability as therapeutic treatments, including for improving solubility of the analogs in near neutral pH aqueous solutions, improving chemical and physical formulation stability, improving peptide membrane permeability in the presence of a permeation enhancer, extending the pharmacokinetic profile, and minimizing potential for injection site reaction or immunogenicity.

It should be noted that the combination of beneficial characteristics of exemplary analogs described herein is not the result of any single modification in isolation but is instead achieved through the novel combinations of the structural features described herein.

In some embodiments, the polypeptides described herein are amidated. In some embodiments, the polypeptides described herein have a modification of the C-terminal group, wherein the modification is NH₂ or absent. In some embodiments, the polypeptides described herein have an OH group at the C-terminal. A nonlimiting list of exemplary therapeutic polypeptides is provided below in Table 1.

TABLE 1
Exemplary Polypeptides

| SEQ ID NO: | Therapeutic Polypeptide |
|---|---|
| 1 | Y-Aib-EGT-αMeF(2F)-TSD-4Pal-SI-αMeL-LDEKAQ-Aib-EFIe-αMeY-LIEGSPSSGAPPPS |
| 2 | Y-Aib-EGT-αMeF(2F)-TSD-4Pal-SI-αMeL-LD-Orn-KAQ-Aib-EFIe-αMeY-LIEGSPSSGAPPPS |
| 3 | Y-Aib-EGT-αMeF(2F)-TSD-4Pal-SI-αMeL-LDEKAQ-Aib-EFIe-αMeY-LIEGGG |
| 4 | Y-Aib-EGT-αMeF(2F)-TSD-4Pal-SI-αMeL-LDEKAQ-Aib-EFIE-αMeY-LIEGSPSSGAPPPS |
| 5 | Y-Aib-EGT-αMeF(2F)-TSD-4Pal-Aib-I-αMeL-LDEKAQ-Aib-EFIE-αMeY-LIEGSPSSGAPPPS |
| 6 | Y-Aib-EGT-αMeF(2F)-TSD-4Pal-Aib-S-αMeL-LDEKAQ-Aib-EFIE-αMeY-LIEGSPSSGAPPPS |
| 7 | Y-Aib-EGT-αMeF(2F)-TSD-4Pal-SI-αMeL-LEEKAQ-Aib-EFIE-αMeY-LIEGSPSSGAPPPS |
| 8 | Y-Aib-EGT-αMeF(2F)-TSDV-Aib-I-αMeL-LD-EKAQ-Aib-EFIe-αMeY-LIEGSPSSGAPPPS |
| 9 | Y-Aib-EGT-αMeF(2F)-TSDV-Aib-I-αMeL-LD-Orn-KAQ-Aib-EFIe-αMeY-LIEGSPSSGAPPPS |
| 10 | Y-Aib-EGT-αMeF(2F)-TSDV-Aib-I-αMeL-LD-Orn-KAQ-Aib-EFIE-αMeY-LIEGSPSSGAPPPS |
| 11 | Y-Aib-EGT-αMeF(2F)-TSD-4Pal-SI-αMeL-LDEKAQ-Aib-EFIK-@MeY-LIEGSPSSGAPPPSGS |
| 12 | Y-Aib-EGT-αMeF(2F)-TSD-4Pal-SI-αMeL-LDEKAQ-Aib-EFIe-αMeY-LIKGSPSSGAPPPSGS |
| 13 | Y-Aib-EGT-αMeF(2F)-TSD-4Pal-SI-αMeL-LDEKAQ-Aib-EFIe-αMeY-LIEGG |
| 14 | Y-Aib-EGT-αMeF(2F)-TSD-4Pal-SI-αMeL-LDEKAQ-Aib-EFIe-αMeY-LIEG |
| 15 | Y-Aib-EGT-αMeF(2F)-TSD-4Pal-SI-αMeL-LDEKAQ-Aib-EFIE-αMeY-LIEGGG |
| 16 | Y-Aib-EGT-αMeF(2F)-TSD-4Pal-Aib-I-αMeL-LDEKAQ-Aib-EFIE-αMeY-LIEGGG |
| 17 | Y-Aib-EGT-αMeF(2F)-TSD-4Pal-SI-αMeL-LEEKAQ-Aib-EFIE-αMeY-LIEGGG |
| 18 | Y-Aib-EGT-αMeF(2F)-TSD-V-Aib-I-αMeL-LDEKAQ-Aib-EFIe-αMeY-LIEGGG |
| 19 | Y-Aib-EGT-αMeF(2F)-TSD-V-Aib-I-αMeL-LD-Orn-KAQ-Aib-EFIe-αMeY-LIEGGG |
| 20 | Y-Aib-EGT-αMeF(2F)-TSD-V-Aib-I-αMeL-LD-Orn-KAQ-Aib-EFIE-αMeY-LIEGGG |
| 21 | Y-Aib-EGT-αMeF(2F)-TSD-4Pal-SI-αMeL-LD-Orn-KAQ-Aib-EFIE-αMeY-LIEGGG |
| 22 | Y-Aib-EGT-αMeF(2F)-TSD-4Pal-Aib-I-αMeL-LD-Orn-KAQ-Aib-EFIE-αMeY-LIEGGG |
| 23 | Y-Aib-EGT-αMeF(2F)-TSD-4Pal-SI-αMeL-LE-Om-KAQ-Aib-EFIE-αMeY-LIEGGG |
| 24 | Y-Aib-EGT-αMeF(2F)-TSD-4Pal-SI-αMeL-LDEKAQ-Aib-EFIE-αMeY-LIKGSPSSGAPPPSGS |
| 25 | Y-Aib-EGT-αMeF(2F)-TSD-4Pal-SI-αMeL-LE-Om-KAQ-Aib-EFIE-αMeY-LIEGSPSSGAPPPS |
| 26 | Y-Aib-EGT-αMeF(2F)-TSD-4Pal-Aib-I-αMeL-LE-Orn-KAQ-Aib-EFIE-αMeY-LIEGGG |
| 27 | Y-Aib-EGT-αMeF(2F)-TSD-4Pal-Aib-I-αMeL-LD-Orn-KAQ-Aib-EFIe-αMeY-LIEGGG |
| 28 | Y-Aib-EGT-αMeF(2F)-TSD-4Pal-SI-αMeL-LD-Om-KAQ-Aib-EFIK-αMeY-LIEGSPSSGAPPPSGS |
| 29 | Y-Aib-EGT-αMeF(2F)-TSD-4Pal-Aib-I-αMeL-LD-Orn-KAQ-Aib-EFIK-αMe Y-LIEGSPSSGAPPPSGS |
| 30 | Y-Aib-EGT-αMeF(2F)-TSD-4Pal-Aib-I-αMeL-LD-Orn-KAQ-Aib-EFIE-αMeY-LIEGGPSSGAPPPSG |
| 31 | Y-Aib-EGT-αMeF(2F)-TSD-4Pal-Aib-I-αMeL-LD-Orn-KAQ-Aib-EFIE-αMeY-LIEGGESSGAPPPSG |
| 32 | Y-Aib-EGT-αMeF(2F)-TSD-4Pal-Aib-I-αMeL-LD-Orn-KAQ-Aib-EFIE-αMeY-LIEGG-Orn-SSGAPPPSG |
| 33 | Y-Aib-EGT-αMeF(2F)-TSD-4Pal-Aib-I-αMeL-LD-Orn-KAQ-Aib-EFIE-αMeY-LIEGGPPSGAPPPSG |
| 34 | Y-Aib-EGT-αMeF(2F)-TSD-4Pal-Aib-I-αMeL-LD-Orn-KAQ-Aib-EFIE-αMeY-LIEGGPPS-Aib-Orn-PPP-Orn-G |
| 35 | Y-Aib-EGT-αMeF(2F)-TSD-4Pal-Aib-I-αMeL-LD-Orn-KAQ-Aib-EFIE-αMeY-LIEGSPSSGAPPPGG |
| 36 | Y-Aib-EGT-αMeF(2F)-TSD-4Pal-Aib-I-αMeL-LD-Orn-KAQ-Aib-EFIK-αMeY-LIEGGPSSGAPPPSGG |
| 37 | Y-Aib-EGT-αMeF(2F)-TSD-4Pal-Aib-I-αMeL-LD-Orn-KAQ-Aib-EFIK-αMeY-LIEGGESSGAPPPSGG |
| 38 | Y-Aib-EGT-αMeF(2F)-TSD-4Pal-Aib-I-αMeL-LD-Orn-KAQ-Aib-EFIK-αMeY-LIEGG-Orn-SSGAPPPSGG |
| 39 | Y-Aib-EGT-αMeF(2F)-TSD-4Pal-Aib-I-αMeL-LD-Orn-KAQ-Aib-EFIK-αMeY-LIEGGPPSGAPPPSGG |
| 40 | Y-Aib-EGT-αMeF(2F)-TSD-4Pal-Aib-I-αMeL-LD-Orn-KAQ-Aib-EFIK-αMeY-LIEGGPPS-Aib-Orn-PPP-Orn-GG |
| 41 | Y-Aib-EGT-αMeF(2F)-TSD-4Pal-Aib-I-αMeL-LD-Orn-KAQ-Aib-EFIK-αMe Y-LIEGSPSSGAPPPGGG |
| 42 | Y-Aib-EGT-αMeF(2F)-TSD-4Pal-Aib-I-αMeL-LD-Orn-KAQ-Aib-EFIE-αMeY-LIEGGPSSG |
| 43 | Y-Aib-EGT-αMeF(2F)-TSD-4Pal-Aib-I-αMeL-LD-Orn-KAQ-Aib-EFIK-αMeY-LIEGGG |
| 44 | Y-Aib-EGT-αMeF(2F)-TSD-4Pal-Aib-I-αMeL-LD-Orn-KAQ-Aib-EFIK-αMeY-LIEGGPSSG |
| 45 | Y-Aib-EGT-αMeF(2F)-TSD-4Pal-Aib-I-αMeL-LD-Orn-KAQ-Aib-EFIE-αMeY-LIKGGG |
| 46 | Y-Aib-EGT-αMeF(2F)-TSD-4Pal-Aib-I-αMeL-LD-Orn-KAQ-Aib-EFIE-αMeY-LIKGGPSSG |
| 47 | Y-Aib-EGT-αMeF(2F)-TSD-4Pal-Aib-I-αMeL-LD-Orn-KAQ-Aib-EFIE-αMeY-LIEGSPSSGAPPPS |
| 48 | Y-Aib-EGT-αMeF(2F)-TSD-4Pal-Aib-I-αMeL-LD-Orn-KAQ-Aib-EFIE-αMeY-LIEGGPSSGAPPPS |
| 49 | Y-Aib-EGT-αMeF(2F)-TSD-4Pal-Aib-I-αMeL-LDEKAQ-Aib-EFIE-αMeY-LIEGGPSSGAPPPS |
| 50 | Y-Aib-EGT-αMeF(2F)-TSD-4Pal-Aib-I-αMeL-LD-Orn-KAQ-Aib-EFIe-αMeY-LIEGSPSSGAPPPS |
| 51 | Y-Aib-EGT-αMeF(2F)-TSD-4Pal-Aib-I-αMeL-LD-Orn-KAQ-Aib-EFIE-αMeY-LIEGG-Orn-SSGAPPPS |
| 52 | Y-Aib-EGT-αMeF(2F)-TSD-4Pal-Aib-I-αMeL-LD-Orn-KAQ-Aib-EFIE-αMeY-LIEGG-Orn |
| 53 | Y-Aib-EGT-αMeF(2F)-TSD-4Pal-Aib-I-αMeL-LD-Orn-KAQ-Aib-Orn-FIE-αMeY-LIEGSPSSGAPPPS |
| 54 | Y-Aib-EGT-αMeF(2F)-TSD-4Pal-Aib-I-αMeL-LD-Orn-KAQ-Aib-EFIQ-αMeY-LIEGSPSSGAPPPS |
| 55 | Y-Aib-EGT-αMeF(2F)-TSD-4Pal-SI-αMeL-LD-Orn-KAQ-Aib-EFIE-αMeY-LIEGSPSSGAPPPS |
| 56 | Y-Aib-EGT-αMeF(2F)-TSD-4Pal-Aib-I-αMeL-LD-Orn-KAQ-Aib-EFIE-αMeY-LIE-Aib-SPSSGAPPPS |
| 57 | Y-Aib-EGT-αMeF(2F)-TSD-4Pal-Aib-I-αMeL-LD-Orn-KAQ-Aib-EFIe-αMeY-LIE-Aib-SPSSGAPPPS |
| 58 | Y-Aib-EGT-αMeF(2F)-TSD-4Pal-SI-αMeL-LD-Om-KAQ-Aib-EFIQ-αMeY-LIEGSPSSGAPPPS |
| 59 | Y-Aib-EGT-αMeF(2F)-TSDY-Aib-I-αMeL-LD-Orn-KAQ-Aib-EFIE-αMeY-LIEGSPSSGAPPPS |
| 60 | Y-Aib-EGT-αMeF(2F)-TSDY-Aib-I-αMeL-LD-Orn-KAQ-Aib-EFIQ-αMeY-LIEGSPSSGAPPPS |
| 61 | Y-Aib-EGT-αMeF(2F)-TSDY-Aib-I-αMeL-LD-Orn-KAQ-Aib-EFIe-αMeY-LIEGSPSSGAPPPS |
| 62 | Y-Aib-EGT-αMeF(2F)-TSD-4Pal-Aib-I-αMeL-LD-Orn-KAQ-αMeL-EFIE-αMeY-LIEGSPSSGAPPPS |
| 63 | Y-Aib-EGT-αMeF(2F)-TSD-4Pal-Aib-I-αMeL-LD-Orn-KAQ-Iva-EFIE-αMeY-LIEGSPSSGAPPPS |
| 64 | Y-Aib-EGT-αMeF(2F)-TSD-4Pal-Aib-I-αMeL-LD-Orn-KAQ-αMeL-EFIQ-αMeY-LIEGSPSSGAPPPS |
| 65 | Y-Aib-EGT-αMeF(2F)-TSD-4Pal-Aib-I-αMeL-LD-Orn-KAQ-Iva-EFIQ-αMeY-LIEGSPSSGAPPPS |
| 66 | Y-Aib-EGT-αMeF(2F)-TSD-4Pal-Aib-I-αMeL-LD-Orn-KAQ-Aib-QFIE-αMeY-LIEGSPSSGAPPPS |
| 67 | Y-Aib-EGT-αMeF(2F)-TSD-4Pal-Aib-I-αMeL-LD-Orn-KAQ-Aib-EFIQ-αMeY-LLEGSPSSGAPPPS |
| 68 | Y-Aib-EGT-αMeF(2F)-TSD-4Pal-SI-αMeL-LD-Orn-KAQ-Aib-EFIQ-αMeY-LIEGGG |
| 69 | Y-Aib-EGT-αMeF(2F)-TSD-4Pal-Aib-I-αMeL-LD-Orn-KAQ-Aib-EFIQ-αMeY-LIEGGG |
| 70 | Y-Aib-EGT-αMeF(2F)-TSD-4Pal-Aib-I-αMeL-LD-Orn-KAQ-Aib-DFIQ-αMeY-LIEGSPSSGAPPPS |
| 71 | Y-Aib-EGT-αMeF(2F)-TSD-4Pal-SI-αMeL-LD-Om-KAQ-Aib-EFIK-αMeY-LIEGSPSSGAPPPS |
| 72 | Y-Aib-EGT-αMeF(2F)-TSD-4Pal-Aib-I-αMeL-LD-Orn-KAQ-Aib-EFIK-αMeY-LIEGSPSSGAPPPS |
| 73 | Y-Aib-EGT-αMeF(2F)-TSDYSI-αMeL-LD-Orn-KAQ-Aib-EFIE-αMeY-LIEGSPSSGAPPPS |
| 74 | Y-Aib-EGT-αMeF(2F)-TSDYSI-αMeL-LD-Om-KAQ-Aib-EFIQ-αMeY-LIEGSPSSGAPPPS |
| 75 | Y-Aib-EGT-αMeF(2F)-TSDY-Aib-I-αMeL-LD-Orn-KAQ-Aib-EFIE-αMeY-LIEGGG |
| 76 | H-Aib-EGT-αMeF(2F)-TSD-4Pal-SI-αMeL-LD-Orn-KAQ-Aib-EFIE-αMeY-LIEGGG |
| 77 | H-Aib-EGT-αMeF(2F)-TSD-4Pal-SI-αMeL-LD-Orn-KAQ-Aib-EFIE-αMeY-LIEGSPSSGAPPPS |
| 78 | Y-Aib-EGT-αMeF(2F)-TSD-4Pal-Aib-I-αMeL-LD-Orn-KAQ-Aib-EFIE-αMeY-LIEQGG |
| 79 | Y-Aib-EGT-αMeF(2F)-TSD-4Pal-SI-αMeL-LD-Orn-KAQ-Aib-EFIe-αMeY-LIEGGPSSGAPPPS |
| 80 | Y-Aib-EGT-αMeF(2F)-TSD-4Pal-Aib-I-αMeL-LD-Orn-KAQ-Aib-EFIe-αMeY-LIEGGPSSGAPPPS |
| 81 | Y-Aib-EGT-αMeF(2F)-TSDY-Aib-I-αMeL-LD-Orn-KAQ-Aib-EFIe-αMeY-LIEGGPSSGAPPPS |
| 82 | Y-Aib-EGT-αMeF(2F)-TSD-4Pal-SI-αMeL-LD-Orn-KAQ-Aib-EFIe-αMeY-LIAGSPSSGAPPPS |
| 83 | Y-Aib-EGT-αMeF(2F)-TSD-4Pal-Aib-I-αMeL-LD-Orn-KAQ-Aib-EFIe-αMeY-LIAGSPSSGAPPPS |
| 84 | Y-Aib-EGT-αMeF(2F)-TSDY-Aib-I-αMeL-LD-Orn-KAQ-Aib-EFIe-αMeY-LIAGSPSSGAPPPS |
| 85 | Y-Aib-EGT-αMeF(2F)-TSD-4Pal-SI-αMeL-LD-Orn-KAQ-Aib-EFIe-αMeY-LIAGGPSSGAPPPS |
| 86 | Y-Aib-EGT-αMeF(2F)-TSDY-Aib-I-αMeL-LD-Orn-KAQ-Aib-EFIe-αMeY-LIAGGPSSGAPPPS |
| 87 | Y-Aib-EGT-αMeF(2F)-TSD-4Pal-SI-αMeL-LD-Orn-KAQ-Aib-EFIe-αMeY-LVEGSPSSGAPPPS |
| 88 | Y-Aib-EGT-αMeF(2F)-TSD-4Pal-SI-αMeL-LD-Orn-KAQ-Aib-EFIE-αMeY-LIAGSPSSGAPPPS |
| 89 | Y-Aib-EGT-αMeF(2F)-TSDY-Aib-I-αMeL-LD-Orn-KAQ-Aib-EFIE-αMeY-LIAGSPSSGAPPPS |
| 90 | Y-Aib-EGT-αMeF(2F)-TSD-4Pal-SI-αMeL-LDEQAQ-Aib-EFIe-αMeY-LIEGSPSSGAPPPS |
| 91 | Y-Aib-EGT-αMeF(2F)-TSD-4Pal-Aib-I-αMeL-LDEKAQ-Aib-EFIE-αMeY-LIEGGPSSG |
| 92 | Y-Aib-EGT-αMeF(2F)-TSD-4Pal-Aib-I-αMeL-LD-Orn-KAQ-Aib-EFIQ-αMeY-LIE-Aib-SPSSGAPPPS |
| 93 | Y-Aib-QGT-αMeF(2F)-TSD-4Pal-SI-αMeL-LD-Orn-KAQ-αMe4Pal-Orn-FIe-αMeY-LIEGSPSSGAPPPS |
| 94 | Y-Aib-QGT-αMeF(2F)-TSD-4Pal-SI-αMeL-LD-Orn-KAQ-αMe4Pal-Orn-FIE-αMeY-LIEGSPSSGAPPPS |
| 95 | Y-Aib-QGT-αMeF(2F)-TSD-4Pal-αMeS-I-αMeL-LD-Orn-KAQ-αMe4Pal-Orn-FIe-αMeY-LIEGSPSSGAPPPS |
| 96 | Y-Aib-QGT-αMeF(2F)-TSD-4Pal-αMeS-I-αMeL-LD-Orn-KAQ-αMe4Pal-Orn-FIE-MeY-LIEGSPSSGAPPPS |
| 97 | Y-Aib-QGT-αMeF(2F)-TSDY-αMeS-I-αMeL-LD-Orn-KAQ-αMe4Pal-Orn-FIe-αMeY-LIEGSPSSGAPPPS |
| 98 | Y-Aib-QGT-αMeF(2F)-TSDY-αMeS-I-αMeL-LD-Orn-KAQ-αMe4Pal-Orn-FIE-αMeY-LIEGSPSSGAPPPS |
| 99 | Y-Aib-QGT-αMeF(2F)-TSD-4Pal-αMeS-I-αMeL-LD-Orn-KAQ-αMe4Pal-Orn-FIE-αMeY-LIEGGG |
| 100 | Y-Aib-QGT-αMeF(2F)-TSDY-αMeS-I-αMeL-LD-Om-KAQ-αMe4Pal-Orn-FIe-αMeY-LIEGGG |
| 101 | Y-Aib-QGT-αMeF(2F)-TSD-4Pal-αMeS-I-αMeL-LD-Orn-KAQ-αMe4Pal-Orn-FIE-αMeY-LIEGGPSSGAPPPS |
| 102 | Y-Aib-QGT-αMeF(2F)-TSDY-αMeS-I-αMeL-LD-Om-KAQ-αMe4Pal-Orn-FIe-αMeY-LIEGGPSSGAPPPS |
| 103 | Y-Aib-QGT-αMeF(2F)-TSD-4Pal-αMeS-I-αMeL-LD-Orn-KAQ-αMe4Pal-Orn-FIK-αMeY-LIEGGPSSGAPPPSGG |
| 104 | Y-Aib-QGT-αMeF(2F)-TSDY-αMeS-I-αMeL-LD-Orn-KAQ-αMe4Pal-Orn-FIK-αMeY-LIEGGPSSGAPPPSGG |
| 105 | Y-Aib-QGT-αMeF(2F)-TSDY-αMeS-I-αMeL-LD-Om-KAQ-αMe4Pal-Orn-FIKαMeY-LIEGGPSSGAPPPSGG |
| 106 | Y-Aib-QGT-αMeF(2F)-TSD-4Pal-αMeS-I-αMeL-LD-Orn-KAQ-αMe4Pal-Orn-FIE-αMeY-LIEGGPSSG |
| 107 | Y-Aib-QGT-αMeF(2F)-TSDY-αMeS-I-αMeL-LD-Orn-KAQ-αMe4Pal-Orn-FIe-αMeY-LIEGGPSSG |
| 108 | Y-Aib-QGT-αMeF(2F)-TSD-4Pal-αMeS-I-αMeL-LD-Orn-KAQ-αMe4Pal-Orn-FIe-αMeY-LIEGSPSSG |
| 109 | Y-Aib-QGT-αMeF(2F)-TSD-4Pal-αMeS-I-αMeL-LD-Orn-KAQ-αMe4Pal-Orn-FIe-αMeY-LIEGGG |
| 110 | Y-Aib-QGT-αMeF(2F)-TSD-4Pal-αMeS-I-αMeL-LD-Orn-KAQ-αMe4Pal-Orn-FIQ-QMeY-LIEGSPSSGAPPPS |
| 111 | Y-Aib-QGT-αMeF(2F)-TSDY-αMeS-I-αMeL-LD-Om-KAQ-αMe4Pal-Orn-FIQ-αMeY-LIEGSPSSGAPPPS |
| 112 | Y-Aib-QGT-αMeF(2F)-TSDY-αMeS-I-αMeL-LD-Om-KAQ-αMe4Pal-Orn-FIe-αMeY-LIE-Aib-SPSSGAPPPS |
| 113 | Y-Aib-QGT-αMeF(2F)-TSDY-αMeS-I-αMeL-LD-Om-KAQ-αMe4Pal-EFIe-αMeY-LIEGSPSSGAPPPS |
| 114 | Y-Aib-QGT-αMeF(2F)-TSDY-αMeS-I-αMeL-LD-Om-KAQ-αMe4Pal-EFIQ-αMeY-LIEGSPSSGAPPPS |
| 115 | Y-Aib-QGT-αMeF(2F)-TSDY-αMeS-I-αMeL-LD-Om-KAQ-Aib-EFIQ-αMeY-LIEGSPSSGAPPPS |
| 116 | Y-Aib-QGT-αMeF(2F)-TSDY-αMeS-I-αMeL-LD-Orn-KAQ-αMe4Pal-EFIQ-QMeY-LLEGSPSSGAPPPS |
| 117 | Y-Aib-QGT-αMeF(2F)-TSD-4Pal-SI-αMeL-LD-Orn-KAQ-αMe4Pal-Orn-FIQ-αMeY-LIEGSPSSGAPPPS |
| 118 | Y-Aib-QGT-αMeF(2F)-TSDY-αMeS-I-αMeL-LD-Orn-KAQ-αMe4Pal-Orn-FIe-αMeY-LIE-Aib-GG |
| 119 | Y-Aib-QaT-αMeF(2F)-TSDY-αMeS-I-αMeL-LD-Orn-KAQ-αMe4Pal-EFIQ-αMeY-LIEGSPSSGAPPPS |
| 120 | Y-αMeS-QGT-αMeF(2F)-TSDY-αMeS-I-αMeL-LD-Orn-KAQ-αMe4Pal-EFIQ-αMeY-LIEGSPSSGAPPPS |
| 121 | Y-Iva-QGT-αMeF(2F)-TSDY-αMeS-I-αMeL-LD-Orn-KAQ-αMe4Pal-EFIQ-αMeY-LIEGSPSSGAPPPS |
| 122 | Y-Aib-HGT-αMeF(2F)-TSDY-αMeS-I-αMeL-LD-Om-KAQ-αMe4Pal-EFIQ-αMeY-LIEGSPSSGAPPPS |
| 123 | H-Aib-HGT-αMeF(2F)-TSDY-αMeS-I-αMeL-LD-Orn-KAQ-αMe4Pal-EFIQ-αMeY-LIEGSPSSGAPPPS |
| 124 | H-Aib-HGT-αMeF(2F)-TSDY-αMeS-I-αMeL-LD-Om-KAQ-Aib-EFIQ-αMeY-LIEGSPSSGAPPPS |
| 125 | H-Aib-HGT-αMeF(2F)-TSD-4Pal-αMeS-I-αMeL-LD-Orn-KAQ-αMe4Pal-EFIQ-αMeY-LIEGSPSSGAPPPS |
| 126 | H-Aib-HGT-αMeF(2F)-TSD-4Pal-SI-αMeL-LD-Orn-KAQ-αMe4Pal-EFIQ-αMeY-LIEGSPSSGAPPPS |
| 127 | Y-Aib-QGT-αMeF(2F)-TSDY-αMeS-I-αMeL-LD-Orn-KAQ-αMe4Pal-EFIQ-αMeY-LIE-Aib-SPSSGAPPPS |
| 128 | Y-Aib-QGT-αMeF(2F)-TSDY-αMeS-I-αMeL-LD-Orn-KAQ-αMe4Pal-EFIE-αMeY-LIEGSPSSGAPPPS |
| 129 | Y-Aib-QGT-αMeF(2F)-TSD-4Pal-αMeS-I-αMeL-LD-Orn-KAQ-αMe4Pal-EFIQ-αMeY-LIEGSPSSGAPPPS |
| 130 | Y-Aib-QGT-αMeF(2F)-TSD-4Pal-αMeS-I-αMeL-LD-Orn-KAQ-αMe4Pal-EFIE-αMeY-LIEGSPSSGAPPPS |
| 131 | Y-Aib-QGT-αMeF(2F)-TSDY-αMeS-I-αMeL-LD-Om-KAQ-αMe4Pal-EFIE-αMeY-LIE-Aib-SPSSGAPPPS |
| 132 | Y-Aib-QGT-αMeF(2F)-TSD-4Pal-αMeS-I-αMeL-LD-Orn-KAQ-αMe4Pal-EFIQ-αMeY-LIE-Aib-SPSSGAPPPS |
| 133 | Y-Aib-QGT-αMeF(2F)-TSD-4Pal-αMeS-I-αMeL-LD-Orn-KAQ-αMe4Pal-EFIE-αMeY-LIE-Aib-SPSSGAPPPS |
| 134 | Y-Aib-QGT-αMeF(2F)-TSDY-αMeS-I-αMeL-LDEKAQ-αMe4Pal-EFIQ-αMeY-LLEGSPSSGAPPPS |
| 135 | Y-Aib-QGT-αMeF(2F)-TSDY-αMeS-I-αMeL-LDEKAQ-αMe4Pal-EFIQ-αMeY-LLE-Aib-SPSSGAPPPS |
| 136 | H-Aib-QGT-αMeF(2F)-TSDY-αMeS-I-αMeL-LD-Om-KAQ-αMe4Pal-EFIQ-αMeY-LIEGSPSSGAPPPS |
| 137 | Y-Aib-QGT-αMeF(2F)-TSDY-αMeS-I-αMeL-LD-Orn-KAQ-αMe4Pal-EFIQ-αMeY-LVEGSPSSGAPPPS |
| 138 | Y-Aib-QGT-αMeF(2F)-TSDY-αMeS-I-αMeL-LD-Om-KAQ-αMe4Pal-EFIQ-αMeY-LIEGGG |
| 139 | H-Aib-HGT-αMeF(2F)-TSDY-αMeS-I-αMeL-LD-Om-KAQ-Aib-EFIQ-αMeY-LLEGSPSSGAPPPS |
| 140 | H-Aib-HGT-αMeF(2F)-TSD-4Pal-SI-αMeL-LD-Orn-KAQ-αMe4Pal-EFIQ-αMeY-LLEGSPSSGAPPPS |
| 141 | Y-Aib-QGT-αMeF(2F)-TSDY-αMeS-I-αMeL-LD-Om-KAQ-αMe4Pal-DFIQ-αMeY-LIEGSPSSGAPPPS |
| 142 | Y-Aib-QGT-αMeF(2F)-TSDY-αMeS-I-αMeL-LD-Om-KAQ-αMe4Pal-Orn-FIE-αMeY-LIA-Aib-SPSSGAPPPS |
| 143 | Y-Aib-QGT-αMeF(2F)-TSDY-αMeS-I-αMeL-LD-Om-KAQ-αMe4Pal-EFIQ-αMeY-LLE-Aib-SPSSGAPPPS |
| 144 | H-Aib-QGT-αMeF(2F)-TSDY-αMeS-I-αMeL-LD-Om-KAQ-αMe4Pal-EFIE-αMeY-LIEGSPSSGAPPPS |
| 145 | H-Aib-QGT-αMeF(2F)-TSDY-αMeS-I-αMeL-LD-Om-KAQ-αMe4Pal-EFIE-αMe Y-LIE-Aib-SPSSGAPPPS |
| 146 | H-Aib-QGT-αMeF(2F)-TSDY-αMeS-I-αMeL-LD-Orn-KAQ-αMe4Pal-EFIE-αMeY-LLEGSPSSGAPPPS |
| 147 | H-Aib-QGT-αMeF(2F)-TSDY-αMeS-I-αMeL-LD-Orn-KAQ-αMe4Pal-EFIE-αMeY-LLE-Aib-SPSSGAPPPS |
| 148 | H-Aib-QGT-αMeF(2F)-TSDY-αMeS-I-αMeL-LD-Om-KAQ-αMe4Pal-EFIQ-αMeY-LIE-Aib-SPSSGAPPPS |
| 149 | H-Aib-QGT-αMeF(2F)-TSDY-αMeS-I-αMeL-LD-Orn-KAQ-αMe4Pal-EFIQ-αMeY-LLE-Aib-SPSSGAPPPS |
| 150 | Y-αMeS-QGT-αMeF(2F)-TSDY-αMeS-I-αMeL-LD-Orn-KAQ-αMe4Pal-EFIE-αMeY-LIEGSPSSGAPPPS |
| 151 | Y-Iva-QGT-αMeF(2F)-TSDY-αMeS-I-αMeL-LD-Orn-KAQ-αMe4Pal-EFIE-αMeY-LIEGSPSSGAPPPS |
| 152 | Y-Ac4c-QGT-αMeF(2F)-TSDY-αMeS-I-αMeL-LD-Orn-KAQ-αMe4Pal-EFIE-αMeY-LIEGSPSSGAPPPS |
| 153 | H-Aib-QGT-αMeF(2F)-TSDY-αMeS-I-αMeL-LD-Orn-KAQ-αMe4Pal-Orn-FIK-αMeY-LLEGSPSSGAPPPS |
| 154 | H-Aib-QGT-αMeF(2F)-TSDY-αMeS-I-αMeL-LD-Om-KAQ-αMe4Pal-EFIKαMeY-LLEGSPSSGAPPPS |
| 155 | H-Aib-QGT-αMeF(2F)-TSDY-αMeS-I-αMeL-LD-Om-KAQ-αMe4Pal-Orn-FIe-αMe Y-LIEGSPSSGAPPPS |
| 156 | H-Aib-QGT-αMeF(2F)-TSDY-αMeS-I-αMeL-LD-Om-KAQ-αMe4Pal-Orn-FIe-αMeY-LLEGSPSSGAPPPS |
| 157 | H-Aib-QGT-αMeF(2F)-TSDY-αMeS-I-αMeL-LD-Om-KAQ-αMe4Pal-Orn-FIE-αMeY-LIEGSPSSGAPPPS |
| 158 | H-Aib-QGT-αMeF(2F)-TSDY-αMeS-I-αMeL-LD-Om-KAQ-αMe4Pal-Orn-FIE-αMeY-LLEGSPSSGAPPPS |
| 159 | Y-Aib-QGT-αMeF(2F)-TSDY-αMeS-I-αMeL-LD-Om-KAQ-αMe4Pal-EFIE-αMeY-LLEGSPSSGAPPPS |
| 160 | Y-Aib-QGT-αMeF(2F)-TSDY-αMeS-I-αMeL-LD-Om-KAQ-αMe4Pal-EFIe-αMeY-LLEGSPSSGAPPPS |
| 161 | Y-Aib-QGT-αMeF(2F)-TSDY-αMeS-I-αMeL-LD-Orn-KAQ-αMe4Pal-Orn-FIE-αMeY-LLEGSPSSGAPPPS |
| 162 | Y-Aib-QGT-αMeF(2F)-TSDY-αMeS-I-αMeL-LD-Om-KAQ-αMe4Pal-Orn-FIe-αMeY-LLEGSPSSGAPPPS |
| 163 | Y-Aib-QGT-αMeF(2F)-TSDY-αMeS-I-αMeL-LD-Orn-KAQ-Aib-QFIE-αMeY-LLEGSPSSGAPPPS |
| 164 | Y-Aib-QGT-αMeF(2F)-TSDY-αMeS-I-αMeL-LD-Orn-KAQ-Aib-Orn-FIe-αMeY-LLEGSPSSGAPPPS |
| 165 | Y-Aib-QGT-αMeF(2F)-TSDY-αMeS-I-αMeL-LD-Orn-KAQ-Aib-Orn-FIE-αMeY-LLEGSPSSGAPPPS |
| 166 | Y-Aib-QGT-αMeF(2F)-TSDY-αMeS-I-αMeL-LD-Orn-KAQ-Aib-Orn-FIq-αMeY-LLEGSPSSGAPPPS |
| 167 | Y-Aib-QGT-αMeF(2F)-TSDY-αMeS-I-αMeL-LD-Orn-KAQ-Aib-Orn-FIQ-αMeY-LLEGSPSSGAPPPS |
| 168 | Y-Aib-QGT-αMeF(2F)-TSDY-αMeS-I-αMeL-LD-Om-KAQ-Aib-EFIe-αMeY-LLEGSPSSGAPPPS |
| 169 | Y-Aib-QGT-αMeF(2F)-TSDY-αMeS-I-αMeL-LD-Om-KAQ-Aib-EFIE-αMeY-LLEGSPSSGAPPPS |
| 170 | Y-Aib-QGT-αMeF(2F)-TSDY-αMeS-I-αMeL-LD-Orn-KAQ-Aib-EFIq-αMeY-LLEGSPSSGAPPPS |
| 171 | H-Ac4c-QGT-αMeF(2F)-TSDY-αMeS-I-αMeL-LD-Orn-KAQ-αMe4Pal-EFIE-αMeY-LIEGSPSSGAPPPS |
| 172 | YaQGT-αMeF(2F)-TSDY-αMeS-I-αMeL-LD-Orn-KAQ-αMe4Pal-EFIE-αMeY-LIEGSPSSGAPPPS |
| 173 | YaQGT-αMeF(2F)-TSDY-αMeS-I-αMeL-LD-Orn-KAQ-αMe4Pal-Orn-FIE-αMeY-LIEGSPSSGAPPPS |
| 174 | YaQGT-αMeF(2F)-TSDY-αMeS-I-αMeL-LD-Orn-KAQ-αMe4Pal-Orn-FIe-αMeY-LIEGSPSSGAPPPS |
| 175 | YaQGT-αMeF(2F)-TSDY-αMeS-I-αMeL-LD-Om-KAQ-αMe4Pal-EFIE-αMeY-LLEGSPSSGAPPPS |
| 176 | YaQGT-αMeF(2F)-TSDY-αMeS-I-αMeL-LD-Orn-KAQ-αMe4Pal-EFIe-αMeY-LLEGSPSSGAPPPS |
| 177 | YaQGT-αMeF(2F)-TSDY-αMeS-I-αMeL-LD-Orn-KAQ-αMe4Pal-Orn-FIE-αMeY-LLEGSPSSGAPPPS |
| 178 | YaQGT-αMeF(2F)-TSDY-αMeS-I-αMeL-LD-Orn-KAQ-Aib-Orn-FIq-αMeY-LLEGSPSSGAPPPS |
| 179 | YaQGT-αMeF(2F)-TSDY-αMeS-I-αMeL-LD-Orn-KAQ-Aib-Orn-FIQ-αMeY-LLEGSPSSGAPPPS |
| 180 | YaQGT-αMeF(2F)-TSDY-αMeS-I-αMeL-LD-Orn-KAQ-Aib-Orn-FIE-αMeY-LIEGSPSSGAPPPS |
| 181 | Y-Aib-QGT-αMeF(2F)-TSD-4Pal-SI-αMeL-LD-Orn-KAQ-αMe4Pal-EFIE-αMeY-LIEGSPSSGAPPPS |
| 182 | Y-Aib-QGT-αMeF(2F)-TSDY-αMeS-I-αMeL-LD-Om-KAQ-Aib-Orn-FIE-αMeY-LIEGSPSSGAPPPS |
| 183 | Y-Aib-QGT-αMeF(2F)-TSD-4Pal-αMeS-I-αMeL-LD-Orn-KAQ-αMe4Pal-EFIe-αMeY-LIEGSPSSGAPPPS |
| 184 | Y-Aib-QGT-αMeF(2F)-TSDY-αMeS-I-αMeL-LD-Om-KAQ-Aib-Orn-FIe-αMeY-LIEGSPSSGAPPPS |
| 185 | NMeY-Aib-QGT-αMeF(2F)-TSDY-αMeS-I-αMeL-LD-Orn-KAQ-αMe4Pal-EFIE-αMeY-LIEGSPSSGAPPPS |
| 186 | Y-Aib-QGT-αMeF(2F)-TSDY-αMeS-I-αMeL-LD-Orn-KAQ-αMe4Pal-Orn-FIQ-αMeY-LIE-Aib-SPSSGAPPPS |
| 996 | Y-Aib-EGT-αMeF(2F)-TSD-4Pal-Aib-I-αMeL-LD-Orn-QAQ-Aib-EFIe-αMeY-LIEGGG |
| 997 | Y-Aib-EGT-αMeF(2F)-TSD-Y-Aib-I-αMeL-LD-Orn-KAQ-Aib-Orn-FIE-αMeY-LIEGSPSSGAPPPS |
| 998 | Y-Aib-EGT-αMeF(2F)-TSDY-Aib-I-αMeL-LD-Orn-KAQ-Aib-EFIE-αMeY-LIAGGG |
| 999 | Y-Aib-EGT-αMeF(2F)-TSDY-Aib-I-αMeL-LD-Orn-KAQ-Aib-EFIE-αMeY-LIE-Aib-GG |
| 1000 | Y-Aib-QGT-αMeF(2F)-TSDY-αMeS-I-αMeL-LD-Orn-KAQ-αMe4Pal-Orn-FIE-αMeY-LVEGSPSSGAPPPS |
| 1001 | Y-Aib-QGT-αMeF(2F)-TSD-4Pal-αMeS-I-αMeL-LD-Orn-KAQ-αMe4Pal-Orn-FIE-αMeY-LVEGSPSSGAPPPS |
| 1002 | Y-Aib-QGT-αMeF(2F)-TSDY-αMeS-I-αMeL-LD-Orn-KAQ-αMe4Pal-EFIE-αMeY-L VEGSPSSGAPPPS |
| 1003 | YaQGT-αMeF(2F)-TSD-4Pal-αMeS-I-QMeL-LD-Orn-KAQ-αMe4Pal-Orn-FIE-αMeY-LIEGSPSSGAPPPS |
| 1004 | YaQGT-αMeF(2F)-TSD-4Pal-SI-αMeL-LD-Orn-KAQ-αMe4Pal-Orn-FIE-αMeY-LIEGSPSSGAPPPS |
| 1005 | YaQGT-αMeF(2F)-TSDY-αMeS-I-αMeL-LD-Orn-KAQ-αMe4Pal-Orn-FIE-αMeY-LIEQSPSSGAPPPS |
| 1006 | Y-αMeS-QGT-αMeF(2F)-TSDY-αMeS-I-αMeL-LD-Om-KAQ-αMe4Pal-Orn-FIE-αMeY-LIEGSPSSGAPPPS |
| 1007 | Y-αMeS-QGT-αMeF(2F)-TSD-4Pal-αMeS-I-αMeL-LD-Om-KAQ-αMe4Pal-Orn-FIE-αMeY-LIEGSPSSGAPPPS |
| 1008 | Y-Iva-QGT-αMeF(2F)-TSDY-αMeS-I-αMeL-LD-Orn-KAQ-αMe4Pal-Orn-FIE-αMeY-LIEGSPSSGAPPPS |
| 1009 | Y-Iva-QGT-αMeF(2F)-TSD-4Pal-αMeS-I-αMeL-LD-Orn-KAQ-αMe4Pal-Orn-FIE-αMeY-LIEGSPSSGAPPPS |
| 1010 | Y-Ac4c-QGT-αMeF(2F)-TSD-4Pal-αMeS-I-αMeL-LD-Orn-KAQ-αMe4Pal-EFIE-αMeY-LIEGSPSSGAPPPS |
| 1011 | Y-Aib-QGT-αMeF(2F)-TSDY-αMeS-I-αMeL-LD-Orn-KAQ-αMeL-Orn-FIe-αMeY-LIEGSPSSGAPPPS |
| 1012 | Y-Aib-QGT-αMeF(2F)-TSDY-αMeS-I-αMeL-LD-Orn-KAQ-αMeL-Iva-FIe-αMeY-LIEGSPSSGAPPPS |
| 1013 | Y-Aib-QGT-αMeF(2F)-TSD-4Pal-αMeS-I-αMeL-LD-Om-KAQ-αMeL-Orn-FIe-αMeY-LIEGSPSSGAPPPS |
| 1014 | Y-Aib-QGT-αMeF(2F)-TSD-4Pal-αMeS-I-αMeL-LD-Orn-KAQ-αMeL-Iva-FIe-αMeY-LIEGSPSSGAPPPS |
| 1015 | Y-Aib-QGT-αMeF(2F)-TSDY-αMeS-I-αMeL-LD-Orn-KAQ-αMe4Pal-EFIE-αMeY-LIAGSPSSGAPPPS |
| 1016 | Y-Aib-QGT-αMeF(2F)-TSDY-αMeS-I-αMeL-LD-Orn-KAQ-αMe4Pal-EFIE-αMeY-LIEaSPSSGAPPPS |
| 1017 | Y-Aib-QGT-αMeF(2F)-TSDY-αMeS-I-αMeL-LD-Orn-KAQ-αMe4Pal-EFIe-αMeY-LIAGSPSSGAPPPS |
| 1018 | YaQGT-αMeF(2F)-TSDY-αMeS-I-αMeL-LD-Orn-KAQ-αMe4Pal-Orn-FIE-αMeY-LIAGSPSSGAPPPS |
| 1019 | Y-Aib-QGT-αMeF(2F)-TSDY-αMeS-I-αMeL-LD-Orn-KAQ-Aib-Orn-FIe-αMeY-LIAGSPSSGAPPPS |
| 1020 | YaQGT-αMeF(2F)-TSDY-αMeS-I-αMeL-LD-Orn-KAQ-αMe4Pal-Orn-FIE-QMeY-LIEaSPSSGAPPPS |
| 1021 | Y-Aib-QGT-αMeF(2F)-TSDY-αMeS-I-αMeL-LD-Orn-KAQ-αMe4Pal-SFIE-αMeY-LIEGSPSSGAPPPS |
| 1022 | Y-Aib-QGT-αMeF(2F)-TSDY-αMeS-I-αMeL-LD-Orn-KAQ-Aib-SFIE-αMeY-LIEGSPSSGAPPPS |
| 1023 | Y-Aib-QGT-αMeF(2F)-TSDY-αMeS-I-αMeL-LD-Orn-KAQ-4Pal-AFIE-αMeY-LIEGSPSSGAPPPS |
| 1024 | Y-Aib-QGT-αMeF(2F)-TSDY-αMeS-I-αMeL-LD-Orn-KAQ-αMeF-Aib-FIe-αMeY-LIEGSPSSGAPPPS |
| 1025 | Y-Aib-QGT-αMeF(2F)-TSDY-αMeS-I-αMeL-LD-Orn-KAQ-Iva-AFIe-αMeY-LIEGSPSSGAPPPS- |
| 1026 | Y-Aib-QGT-αMeF(2F)-TSDY-αMeS-I-αMeL-LD-Orn-KAQ-Aib-EFIE-αMeY-LIEGSPSSGAPPPS |
| 1027 | Y-Aib-QGT-αMeF(2F)-TSDY-αMeS-I-αMeL-LD-Orn-KAQ-αMe4Pal-EFIe-αMeY-LIE-Aib-SPSSGAPPPS |
| 1028 | Y-Aib-QGT-αMeF(2F)-TSDY-αMeS-I-αMeL-LD-Orn-KAQ-αMeL-Orn-FIe-αMeY-LIAaSPSSGAPPPS- |
| 1029 | Y-Aib-QGT-αMeF(2F)-TSD-4Pal-αMeS-I-αMeL-LD-Orn-KAQ-αMe4Pal-EFIE-αMeY-LIAGSPSSGAPPPS |
| 1030 | Y-Aib-QGT-αMeF(2F)-TSD-4Pal-αMeS-I-αMeL-LD-Orn-KAQ-αMe4Pal-EFIE-αMeY-LIAaSPSSGAPPPS |
| 1031 | Y-Aib-QGT-αMeF(2F)-TSD-4Pal-αMeS-I-αMeL-LD-Orn-KAQ-αMeL-Iva-FIe-αMeY-LIA-Aib-SPSSGAPPPS |
| 1032 | Y-Aib-QGT-αMeF(2F)-TSD-4Pal-αMeS-I-αMeL-LD-Orn-KAQ-αMe4Pal-SFIE-αMeY-LIEGSPSSGAPPPS |
| 1033 | Y-Aib-QGT-αMeF(2F)-TSD-4Pal-αMeS-I-αMeL-LD-Orn-KAQ-αMe4Pal-SFIE-αMeY-LIE-Aib-SPSSGAPPPS |
| 1034 | Y-Aib-QGT-αMeF(2F)-TSDY-αMeS-I-αMeL-LD-Orn-KAQ-αMe4Pal-EFIE-αMeY-LIEGGG |
| 1035 | Y-Aib-QGT-αMeF(2F)-TSDY-αMeS-I-αMeL-LD-Orn-KAQ-αMe4Pal-EFIE-αMeY-LIAGGG |
| 1036 | Y-Aib-QGT-αMeF(2F)-TSDY-αMeS-I-αMeL-LD-Orn-KAQ-αMe4Pal-EFIe-αMeY-LIEGGG |
| 1037 | Y-Ac4c-QGT-αMeF(2F)-TSDY-αMeS-I-αMeL-LD-Orn-KAQ-αMe4Pal-EFIE-αMeY-LIEGGG |
| 1038 | Y-Aib-QGT-αMeF(2F)-TSD-4Pal-αMeS-I-αMeL-LD-Orn-KAQ-αMe4Pal-EFIE-αMeY-LIEGGG |

### Acylation of Therapeutic Polypeptides

As noted before, in some embodiments, certain polypeptides described herein include an albumin-binding moiety, such as a fatty acid moiety conjugated, for example, by way of a direct bond or a linker to a natural or non-natural amino acid with a functional group available for conjugation. Such a conjugation is sometimes referred to as acylation. In certain instances, the amino acid with a functional group available for conjugation can be K, C, E or D. In particular instances, the amino acid with a functional group available for conjugation is K, where the conjugation is to an epsilon-amino group of a K sidechain.

In some embodiments, the polypeptides described herein utilize a C₁₆-C₂₂ fatty acid chemically conjugated to the functional group of an amino acid either via a direct bond or via a linker. The length and composition of the fatty acid impacts half-life of the polypeptides, their potency in in vivo animal models, and their solubility and stability. Conjugation to a C₁₆-C₂₂ saturated fatty monoacid or diacid results in polypeptides that exhibit desirable half-life, desirable potency in in vivo animal models, and desirable solubility and stability characteristics.

Examples of saturated C₁₆-C₂₂ fatty acids for use herein include, but are not limited to, palmitic acid (hexadecanoic acid) (C₁₆ monoacid), hexadecanedioic acid (C₁₆ diacid), margaric acid (heptadecanoic acid)(C₁₇ monoacid), heptadecanedioic acid (C₁₇ diacid), stearic acid (Cis monoacid), octadecanedioic acid (C₁₈ diacid), nonadecylic acid (nonadecanoic acid)(C₁₉ monoacid), nonadecanedioic acid (C₁₉ diacid), arachidic acid (eicosanoic acid)(C₂₀ monoacid), eicosanedioic acid (C₂₀ diacid), heneicosylic acid (heneicosanoic acid)(C₂₁ monoacid), heneicosanedioic acid (C₂₁ diacid), behenic acid (docosanoic acid)(C₂₂ monoacid), docosanedioic acid (C₂₂ diacid), including branched and substituted derivatives thereof.

In certain instances, the C₁₆-C₂₂ fatty acid can be a saturated C₁₈ monoacid, a saturated C₁₈ diacid, a saturated C₁₉ monoacid, a saturated C₁₉ diacid, a saturated C₂₀ monoacid, a saturated C₂₀ diacid, and branched and substituted derivatives thereof. In more particular instances, the C₁₆-C₂₂ fatty acid can be octadecanedioic (C₁₈ diacid) or eicosanedioic acid (C₂₀ diacid).

In some embodiments, a lysine at position 17 is conjugated to the fatty acid. In some embodiments the fatty acid is a C₁₆-C₂₂ fatty acid.

In some embodiments, the therapeutic polypeptide is conjugated to the fatty acid by way of a linker. In some embodiments, the linker comprises one to five amino acids. In instances in which the linker includes at least one amino acid, the amino acid can be one to five Glu or γGlu amino acid residues. In some instances, the linker can include one or two or three or four or five Glu or γGlu amino acid residues, including the D-forms thereof. For example, the linker can include either one or two or three or four γGlu amino acid residues. Alternatively, the linker can include one to five amino acid residues (such as, for example, Glu or γGlu amino acids) used in combination with one to five (2-[2-(2-amino-ethoxy)-ethoxy]-acetyl) (“AEEA”) and/or one to five εK moieties. Specifically, the linker can be combinations of one to five Glu or γGlu amino acids and one to five (2-[2-(2-amino-ethoxy)-ethoxy]-acetyl) moieties, or one to five Glu or γGlu amino acids and one to five εK moieties. In some instances, the linker can be combinations of one or two or three γGlu amino acids and one or two (2-[2-(2-amino-ethoxy)-ethoxy]-acetyl) or εK moieties.

For example, in some embodiments the polypeptides described herein have linker and fatty acid components having the structure of the following formula:

(γGlu)ₐ-(2-[2-(2-amino-ethoxy)-ethoxy]-acetyl)_{b}-(γGlu)_{c}-CO—(CH₂)ₚ—CO₂H, where a is 0, 1 or 2; b is 0, 1 or 2; c is 0, 1, 2 or 3; and p is an integer between 14 to 20.

In some preferred embodiments, a is 0 or 1; b is 0, 1 or 2; c is 1, 2 or 3; and p an integer between 14 to 20.

In some embodiments, a is 0, b is 1, c is 1 or 2 and p is 16 or 18.

For example, in some embodiments, a is 0, b is 1, c is 1 and p is 16, the structure of which is depicted below.

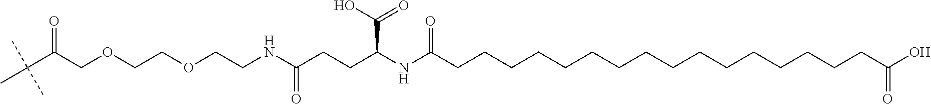

For example, in some embodiments, a is 0, b is 1, c is 1 and p is 18, the structure of which is depicted below.

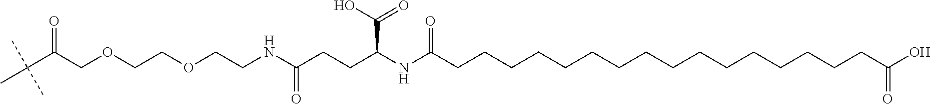

In some embodiments, a is 0, b is 1, c is 2 and p is 16, the structure of which is depicted below.

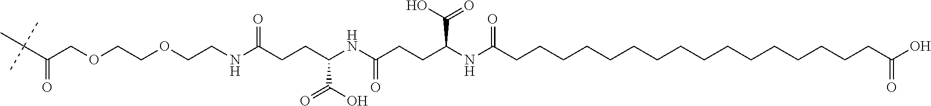

In some embodiments, a is 0, b is 1, c is 2 and p is 18, the structure of which is depicted below.

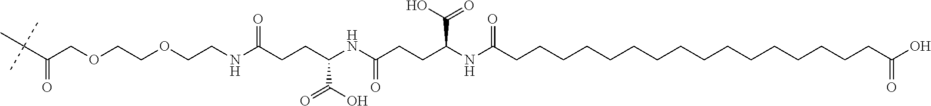

In some embodiments, a is 0, b is 2, c is 1 and p is 16 or 18.

For example, in some embodiments, a is 0, b is 2, c is 1 and p is 16, the structure of which is depicted below.

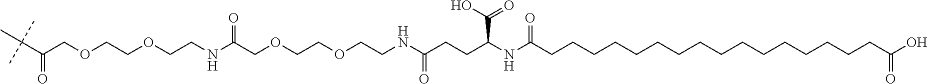

In some embodiments, a is 0, b is 2, c is 1 and p is 18, the structure of which is depicted below.

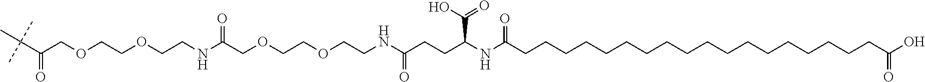

In some embodiments, a is 0, b is 0, c is 2 and p is 16 or 18.

For example, in some embodiments, a is 0, b is 0, c is 2 and p is 16, the structure of which is depicted below.

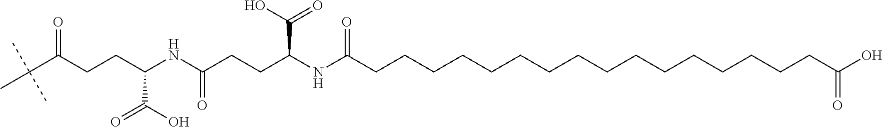

In some embodiments, a is 0, b is 0, c is 2 and p is 18, the structure of which is depicted below.

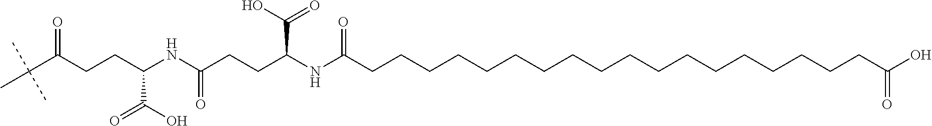

In some embodiments, a is 0, b is 0, c is 3 and p is 16 or 18.

For example, in some embodiments, a is 0, b is 0, c is 3 and p is 16, the structure of which is depicted below.

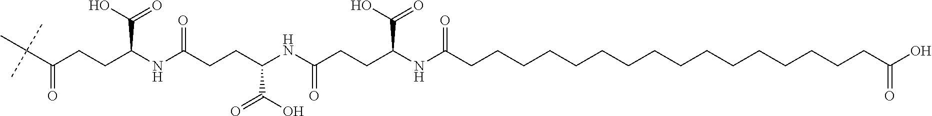

In some embodiments, a is 0, b is 0, c is 3 and p is 18, the structure of which is depicted below.

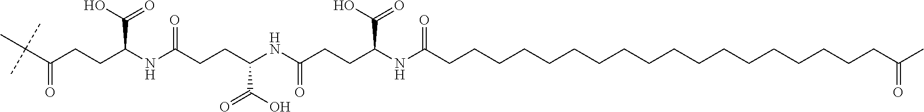

In some embodiments, a is 1, b is 1, c is 1 and p is 16 or 18.

For example, in some embodiments, a is 1, b is 1, c is 1 and p is 16, the structure of which is depicted below.

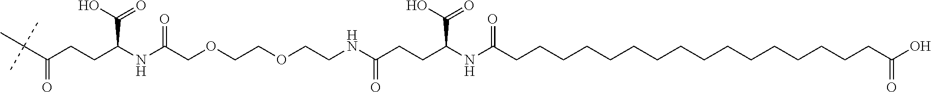

For example, in some embodiments, a is 1, b is 1, c is 1 and p is 18, the structure of which is depicted below.

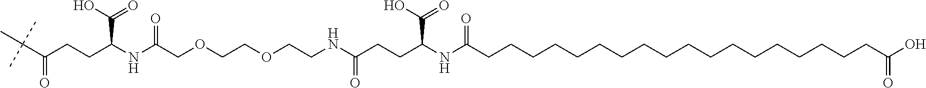

In some embodiments the polypeptides described herein have linker and fatty acid components having the structure of the following formula:

(γGlu)_{d}-(εK)ₑ-(γGlu)_{f}-CO—(CH₂)_{q}—CO₂H, where d is 0, 1 or 2; e is 0, 1 or 2; f is 0, 1, 2 or 3; and q is an integer between 14 to 20.

For example, in one embodiment, d is 0; e is 2; f is 1; and q an integer between 14 to 20. In some embodiments, d is 0; e is 2; f is 1; and q is 16 or 18.

For example, in some embodiments, d is 0; e is 2; f is 1; and q is 16, the structure of which is depicted below.

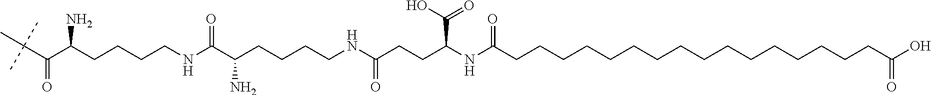

For example, in some embodiments, d is 0; e is 2; f is 1; and q is 18, the structure of which is depicted below.

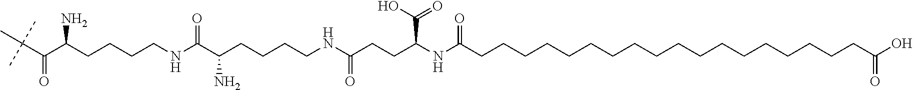

A nonlimiting list of exemplary acylated therapeutic polypeptides is provided below in Table 2.

TABLE 2
Therapeutic Polypeptides Conjugated to a Fatty Acid

| SEQ | |
|---|---|
| ID | Therapeutic Polypeptides |
| NO: | Conjugated to a Fatty Acid |
| 187 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIe-αMeY-LIEGSPSSGAPPPS |
| 188 | Y-Aib-EGT-αMeF(2F)-TSD-4Pal-SI-αMeL-LD-Om-K(2-[2-(2-amino-ethoxy)-ethoxy]-acetyl)₂-(γGlu)-CO- |
| | (CH₂)₁₆-CO₂H)AQ-Aib-EFIe-αMeY-LIEGSPSSGAPPPS |
| 189 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIe-αMeY-LIEGGG |
| 190 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIE-αMeY-LIEGSPSSGAPPPS |
| 191 | Y-Aib-EGT-αMeF(2F)-TSD-4Pal-Aib-I-αMeL-LDEK(2-[2-(2-amino-ethoxy)-ethoxy]-acetyl)₂-(γGlu)-CO-(CH₂)₁₆- |
| | CO₂H)AQ-Aib-EFIE-αMeY-LIEGSPSSGAPPPS |
| 192 | Y-Aib-EGT-αMeF(2F)-TSD-4Pal-Aib-S-αMeL-LDEK(2-[2-(2-amino-ethoxy)-ethoxy]-acetyl)₂-(γGlu)-CO- |
| | (CH₂)₁₆-CO₂H)AQ-Aib-EFIE-αMeY-LIEGSPSSGAPPPS |
| 193 | Y-Aib-EGT-αMeF(2F)-TSD-4Pal-SI-αMeL-LEEK(2-[2-(2-amino-ethoxy)-ethoxy]-acetyl)₂-(γGlu)-CO-(CH₂)₁₆- |
| | CO₂H)AQ-Aib-EFIE-αMeY-LIEGSPSSGAPPPS |
| 194 | Y-Aib-EGT-αMeF(2F)-TSDV-Aib-I-αMeL-LD-EK(2-[2-(2-amino-ethoxy)-ethoxy]-acetyl)₂-(γGlu)-CO-(CH₂)₁₆- |
| | CO₂H)AQ-Aib-EFIe-αMeY-LIEGSPSSGAPPPS |
| 195 | Y-Aib-EGT-αMeF(2F)-TSDV-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIe-αMeY-LIEGSPSSGAPPPS |
| 196 | Y-Aib-EGT-αMeF(2F)-TSDV-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEGSPSSGAPPPS |
| 197 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIK-αMeY-LIEGSPSSGAPPPSGS |
| 198 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIe-αMeY-LIKGSPSSGAPPPSGS |
| 199 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIe-αMeY-LIEGG |
| 200 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIe-αMeY-LIEG |
| 201 | Y-Aib-EGT-αMeF(2F)-TSD-4Pal-SI-αMeL-LDEK(ϵKϵK-(γGlu)-CO-(CH₂)₁₆-CO₂H)AQ-Aib-EFIe-αMeY- |
| | LIEGSPSSGAPPPS |
| 202 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIE-αMeY-LIEGGG |
| 203 | Y-Aib-EGT-αMeF(2F)-TSD-4Pal-Aib-I-αMeL-LDEK(2-[2-(2-amino-ethoxy)-ethoxy]-acetyl)₂-(γGlu)-CO-(CH₂)₁₆- |
| | CO₂H)AQ-Aib-EFIE-αMeY-LIEGGG |
| 204 | Y-Aib-EGT-αMeF(2F)-TSD-4Pal-SI-αMeL-LEEK(2-[2-(2-amino-ethoxy)-ethoxy]-acetyl)₂-(γGlu)-CO-(CH₂)₁₆- |
| | CO₂H)AQ-Aib-EFIE-αMeY-LIEGGG |
| 205 | Y-Aib-EGT-αMeF(2F)-TSD-V-Aib-I-αMeL-LDEK(2-[2-(2-amino-ethoxy)-ethoxy]-acetyl)₂-(γGlu)-CO-(CH₂)₁₆- |
| | CO₂H)AQ-Aib-EFIe-αMeY-LIEGGG |
| 206 | Y-Aib-EGT-αMeF(2F)-TSD-V-Aib-I-αMeL-LD-Orn-K(2-[2-(2-amino-ethoxy)-ethoxy]-acety1)₂-(γGlu)-CO- |
| | (CH₂)₁₆-CO₂H)AQ-Aib-EFIe-αMeY-LIEGGG |
| 207 | Y-Aib-EGT-αMeF(2F)-TSD-V-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGG |
| 208 | Y-Aib-EGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGG |
| 209 | Y-Aib-EGT-αMeF(2F)-TSD-4Pal-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGG |
| 210 | Y-Aib-EGT-αMeF(2F)-TSD-4Pal-SI-αMeL-LE-Orn-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGG |
| 211 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₆- |
| | CO₂H)AQ-Aib-EFIE-αMeY-LIKGSPSSGAPPPSGS |
| 212 | Y-Aib-EGT-αMeF(2F)-TSD-4Pal-SI-αMeL-LE-Orn-K(2-[2-(2-amino-ethoxy)-ethoxy]-acetyl)₂-(γGlu)-CO- |
| | (CH₂)₁₆-CO₂H)AQ-Aib-EFIE-αMeY-LIEGSPSSGAPPPS |
| 213 | Y-Aib-EGT-αMeF(2F)-TSD-4Pal-Aib-I-αMeL-LE-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGG |
| 214 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIe-αMeY-LIEGGG |
| 215 | Y-Aib-EGT-αMeF(2F)-TSD-4Pal-SI-αMeL-LD-Om-K(2-[2-(2-amino-ethoxy)-ethoxy]-acetyl)₂-(γGlu)-CO- |
| | (CH₂)₁₆-CO₂H)AQ-Aib-EFIK-αMeY-LIEGSPSSGAPPPSGS |
| 216 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIK-αMeY-LIEGSPSSGAPPPSGS |
| 217 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIEGGPSSGAPPPSG |
| 218 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIEGGESSGAPPPSG |
| 219 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIE-αMeY- |
| | LIEGG-Om-SSGAPPPSG |
| 220 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIEGGPPSGAPPPSG |
| 221 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIE-αMeY- |
| | LIEGGPPS-Aib-Orn-PPP-Orn-G |
| 222 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIEGSPSSGAPPPGG |
| 223 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIK-αMeY- |
| | LIEGGPSSGAPPPSGG |
| 224 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIK-αMeY- |
| | LIEGGESSGAPPPSGG |
| 225 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIK-αMeY- |
| | LIEGG-Orn-SSGAPPPSGG |
| 226 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIK-αMeY- |
| | LIEGGPPSGAPPPSGG |
| 227 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIK-αMeY- |
| | LIEGGPPS-Aib-Orn-PPP-Or-GG |
| 228 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIK-αMeY- |
| | LIEGSPSSGAPPPGGG |
| 229 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIE-αMeY- |
| | LIEGGG |
| 230 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIEGGPSSG |
| 231 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIK-αMeY- |
| | LIEGGG |
| 232 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIK-αMeY- |
| | LIEGGPSSG |
| 233 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIE-αMeY- |
| | LIKGGG |
| 234 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIKGGPSSG |
| 235 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIK-αMeY-LIEGGG |
| 236 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIK-αMeY-LIEGGPSSG |
| 237 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIE-αMeY-LIKGGG |
| 238 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIE-αMeY-LIKGGPSSG |
| 239 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGPSSG |
| 240 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEGSPSSGAPPPS |
| 241 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIE-αMeY- |
| | LIEGSPSSGAPPPS |
| 242 | Y-Aib-EGT-αMeF(2F)-TSD-4Pal-Aib-I-αMeL-LDEK(ϵKϵK-(γGlu)-CO-(CH₂)₁₆-CO₂H)AQ-Aib-EFIE-αMeY- |
| | LIEGSPSSGAPPPS |
| 243 | Y-Aib-EGT-αMeF(2F)-TSD-4Pal-Aib-I-αMeL-LD-Or-K(2-[2-(2-amino-ethoxy)-ethoxy]-acetyl)-(γGlu)-CO- |
| | (CH₂)₁₆-CO₂H)AQ-Aib-EFIE-αMeY-LIEGGPSSGAPPPS |
| 244 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIEGGPSSGAPPPS |
| 245 | Y-Aib-EGT-αMeF(2F)-TSD-4Pal-Aib-I-αMeL-LDEK(ϵKϵK-(γGlu)-CO-(CH₂)₁₆-CO₂H)AQ-Aib-EFIE-αMeY- |
| | LIEGGPSSGAPPPS |
| 246 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIe-αMeY- |
| | LIEGSPSSGAPPPS |
| 247 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIe-αMeY- |
| | LIEGGG |
| 248 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIE-αMeY- |
| | LIEGG-Om-SSGAPPPS |
| 249 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEGG-Om-SSGAPPPS |
| 250 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIE-αMeY- |
| | LIEGG-Om |
| 251 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIE-αMeY-LIEGG-Orn |
| 252 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-Orn-FIE- |
| | αMeY-LIEGSPSSGAPPPS |
| 253 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-Orn-FIE-αMeY-LIEGSPSSGAPPPS |
| 254 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIQ-αMeY-LIEGSPSSGAPPPS |
| 255 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIQ-αMeY- |
| | LIEGSPSSGAPPPS |
| 256 | Y-Aib-EGT-αMeF(2F)-TSD-4Pal-SI-αMeL-LD-Om-K(2-[2-(2-amino-ethoxy)-ethoxy]-acetyl)₂-(γGlu)-CO- |
| | (CH₂)₁₆-CO₂H)AQ-Aib-EFIE-αMeY-LIEGSPSSGAPPPS |
| 257 | Y-Aib-EGT-αMeF(2F)-TSD-4Pal-SI-αMeL-LD-Om-K(ϵKϵK-(γGlu)-CO-(CH₂)₁₆-CO₂H)AQ-Aib-EFIE-αMeY- |
| | LIEGSPSSGAPPPS |
| 258 | Y-Aib-EGT-αMeF(2F)-TSD-4Pal-SI-αMeL-LD-Om-K(ϵKϵK-(γGlu)-CO-(CH₂)₁₆-CO₂H)AQ-Aib-EFIe-αMeY- |
| | LIEGSPSSGAPPPS |
| 259 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIE-Aib-SPSSGAPPPS |
| 260 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIE-αMeY- |
| | LIE-Aib-SPSSGAPPPS |
| 261 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIe-αMeY- |
| | LIE-Aib-SPSSGAPPPS |
| 262 | Y-Aib-EGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₆-CO₂H)AQ-Aib-EFIQ-αMeY-LIEGSPSSGAPPPS |
| 263 | Y-Aib-EGT-αMeF(2F)-TSD-4Pal-SI-αMeL-LD-Om-K(ϵKϵK-(γGlu)-CO-(CH₂)₁₆-CO₂H)AQ-Aib-EFIQ-αMeY- |
| | LIEGSPSSGAPPPS |
| 264 | Y-Aib-EGT-αMeF(2F)-TSDY-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIE-αMeY-LIEGSPSSGAPPPS |
| 265 | Y-Aib-EGT-αMeF(2F)-TSDY-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIQ-αMeY-LIEGSPSSGAPPPS |
| 266 | Y-Aib-EGT-αMeF(2F)-TSDY-Aib-I-αMeL-LD-Or-K(ϵKϵK-(γGlu)-CO-(CH₂)₁₆-CO₂H)AQ-Aib-EFIe-αMeY- |
| | LIEGSPSSGAPPPS |
| 267 | Y-Aib-EGT-αMeF(2F)-TSDY-Aib-I-αMeL-LD-Or-K(ϵKϵK-(γGlu)-CO-(CH₂)₁₆-CO₂H)AQ-Aib-EFIE-αMeY- |
| | LIEGSPSSGAPPPS |
| 268 | Y-Aib-EGT-αMeF(2F)-TSDY-Aib-I-αMeL-LD-Or-K(ϵKϵK-(γGlu)-CO-(CH₂)₁₆-CO₂H)AQ-Aib-EFIQ-αMeY- |
| | LIEGSPSSGAPPPS |
| 269 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-αMeL-EFIE- |
| | αMeY-LIEGSPSSGAPPPS |
| 270 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Iva-EFIE-αMeY- |
| | LIEGSPSSGAPPPS |
| 271 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-αMeL-EFIQ-αMeY-LIEGSPSSGAPPPS |
| 272 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Iva-EFIQ-αMeY-LIEGSPSSGAPPPS |
| 273 | Y-Aib-EGT-αMeF(2F)-TSD-4Pal-Aib-I-αMeL-LD-Om-K(ϵKϵK-(γGlu)-CO-(CH₂)₁₆-CO₂H)AQ-αMeL-EFIQ- |
| | αMeY-LIEGSPSSGAPPPS |
| 274 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Iva-EFIQ-αMeY- |
| | LIEGSPSSGAPPPS |
| 275 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-QFIE-αMeY-LIEGSPSSGAPPPS |
| 276 | Y-Aib-EGT-αMeF(2F)-TSD-4Pal-Aib-I-αMeL-LD-Or-K(ϵKϵK-(γGlu)-CO-(CH₂)₁₆-CO₂H)AQ-Aib-EFIQ-αMeY- |
| | LLEGSPSSGAPPPS |
| 277 | Y-Aib-EGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₆-CO₂H)AQ-Aib-EFIQ-αMeY-LIEGGG |
| 278 | Y-Aib-EGT-αMeF(2F)-TSD-4Pal-SI-αMeL-LD-Or-K(ϵKϵK-(γGlu)-CO-(CH₂)₁₆-CO₂H)AQ-Aib-EFIE-αMeY- |
| | LIEGGG |
| 279 | Y-Aib-EGT-αMeF(2F)-TSD-4Pal-SI-αMeL-LD-Or-K(ϵKϵK-(γGlu)-CO-(CH₂)₁₆-CO₂H)AQ-Aib-EFIQ-αMeY- |
| | LIEGGG |
| 280 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIQ-αMeY-LIEGGG |
| 281 | Y-Aib-EGT-αMeF(2F)-TSD-4Pal-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-DFIQ-αMeY-LIEGSPSSGAPPPS |
| 282 | Y-Aib-EGT-αMeF(2F)-TSD-4Pal-SI-αMeL-LD-Om-K(2-[2-(2-amino-ethoxy)-ethoxy]-acetyl)₂-(γGlu)-CO- |
| | (CH₂)₁₆-CO₂H)AQ-Aib-EFIK-αMeY-LIEGSPSSGAPPPS |
| 283 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIK-αMeY-LIEGSPSSGAPPPS |
| 284 | Y-Aib-EGT-αMeF(2F)-TSD-4Pal-Aib-I-αMeL-LD-Or-K(eK-2-[2-(2-amino-ethoxy)-ethoxy]-acetyl)-γGlu- CO- |
| | (CH₂)₁₆-CO₂H)AQ-Aib-EFIQ-αMeY-LIEGSPSSGAPPPS |
| 285 | Y-Aib-EGT-αMeF(2F)-TSD-4Pal-Aib-I-αMeL-LD-Orn-K(2-[2-(2-amino-ethoxy)-ethoxy]-acetyl)- ek-γGlu- CO- |
| | (CH₂)₁₆-CO₂H)AQ-Aib-EFIQ-αMeY-LIEGSPSSGAPPPS |
| 286 | Y-Aib-EGT-αMeF(2F)-TSDYSI-αMeL-LD-Om-K(2-[2-(2-amino-ethoxy)-ethoxy]-acetyl)₂-(γGlu)-CO-(CH₂)₁₆- |
| | CO₂H)AQ-Aib-EFIE-αMeY-LIEGSPSSGAPPPS |
| 287 | Y-Aib-EGT-αMeF(2F)-TSDYSI-αMeL-LD-Orn-K(2-[2-(2-amino-ethoxy)-ethoxy]-acetyl)₂-(γGlu)-CO-(CH₂)₁₆- |
| | CO₂H)AQ-Aib-EFIQ-αMeY-LIEGSPSSGAPPPS |
| 288 | Y-Aib-EGT-αMeF(2F)-TSDYSI-αMeL-LD-Om-K(ϵKϵK-(γGlu)-CO-(CH₂)₁₆-CO₂H)AQ-Aib-EFIE-αMeY- |
| | LIEGSPSSGAPPPS |
| 289 | Y-Aib-EGT-αMeF(2F)-TSDYSI-αMeL-LD-Or-K(ϵKϵK-(γGlu)-CO-(CH₂)₁₆-CO₂H)AQ-Aib-EFIQ-αMeY- |
| | LIEGSPSSGAPPPS |
| 290 | Y-Aib-EGT-αMeF(2F)-TSD-4Pal-Aib-I-αMeL-LD-Om-K ϵKϵK-(γGlu)-CO-(CH₂)₁₆-CO₂H)AQ-Aib-EFIQ-αMeY- |
| | LIEGGG |
| 291 | Y-Aib-EGT-αMeF(2F)-TSD-4Pal-SI-αMeL-LD-Om-KϵKϵK-(γGlu)-CO-(CH₂)₁₆-CO₂H)AQ-Aib-EFIQ-αMeY- |
| | LIEGGG |
| 292 | Y-Aib-EGT-αMeF(2F)-TSDY-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGG |
| 293 | H-Aib-EGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEGGG |
| 294 | H-Aib-EGT-αMeF(2F)-TSD-4Pal-SI-αMeL-LD-Or-K(ϵKϵK-(γGlu)-CO-(CH₂)₁₆-CO₂H)AQ-Aib-EFIE-αMeY- |
| | LIEGSPSSGAPPPS |
| 295 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIE-αMeY-LIEQGG |
| 296 | Y-Aib-EGT-αMeF(2F)-TSD-4Pal-SI-αMeL-LD-Or-K(ϵKϵK-(γGlu)-CO-(CH₂)₁₆-CO₂H)AQ-Aib-EFIe-αMeY- |
| | LIEGGPSSGAPPPS |
| 297 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIe-αMeY- |
| | LIEGGPSSGAPPPS |
| 298 | Y-Aib-EGT-αMeF(2F)-TSDY-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIe-αMeY-LIEGGPSSGAPPPS |
| 299 | Y-Aib-EGT-αMeF(2F)-TSDY-Aib-I-αMeL-LD-Om-K(ϵKϵK-(γGlu)-CO-(CH₂)₁₆-CO₂H)AQ-Aib-EFIe-αMeY- |
| | LIEGGPSSGAPPPS |
| 300 | Y-Aib-EGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₆-CO₂H)AQ-Aib-EFIe-αMeY-LIAGSPSSGAPPPS |
| 301 | Y-Aib-EGT-αMeF(2F)-TSD-4Pal-SI-αMeL-LD-Om-KAQ-Aib-EFIe-αMeY-LIAGSPSSGAPPPS |
| 302 | Y-Aib-EGT-αMeF(2F)-TSD-4Pal-Aib-I-αMeL-LD-Om-K(ϵKϵK-(γGlu)-CO-(CH₂)₁₆-CO₂H)AQ-Aib-EFIe-αMeY- |
| | LIAGSPSSGAPPPS |
| 303 | Y-Aib-EGT-αMeF(2F)-TSDY-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIe-αMeY-LIAGSPSSGAPPPS |
| 304 | Y-Aib-EGT-αMeF(2F)-TSDY-Aib-I-αMeL-LD-Orn-K(ϵKϵK-(γGlu)-CO-(CH₂)₁₆-CO₂H)AQ-Aib-EFIe-αMeY- |
| | LIAGSPSSGAPPPS |
| 305 | Y-Aib-EGT-αMeF(2F)-TSD-4Pal-SI-αMeL-LD-Or-K(ϵKϵK-(γGlu)-CO-(CH₂)₁₆-CO₂H)AQ-Aib-EFIe-αMeY- |
| | LIAGGPSSGAPPPS |
| 306 | Y-Aib-EGT-αMeF(2F)-TSD-4Pal-SI-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIe-αMeY-LIAGGPSSGAPPPS |
| 307 | Y-Aib-EGT-αMeF(2F)-TSDY-Aib-I-αMeL-LD-Om-K(ϵKϵK-(γGlu)-CO-(CH₂)₁₆-CO₂H)AQ-Aib-EFIe-αMeY- |
| | LIAGGPSSGAPPPS |
| 308 | Y-Aib-EGT-αMeF(2F)-TSDY-Aib-I-αMeL-LD-Om-K(2-[2-(2-amino-ethoxy)-ethoxy]-acetyl)₂-(γGlu)-CO- |
| | (CH₂)₁₆-CO₂H)AQ-Aib-EFIe-αMeY-LIAGGPSSGAPPPS |
| 309 | Y-Aib-EGT-αMeF(2F)-TSD-4Pal-SI-αMeL-LD-Om-K(2-[2-(2-amino-ethoxy)-ethoxy]-acetyl)-(γGlu)-CO-(CH₂)₁₆- |
| | CO₂H)AQ-Aib-EFIe-αMeY-LIEGSPSSGAPPPS |
| 310 | Y-Aib-EGT-αMeF(2F)-TSDY-Aib-I-αMeL-LD-Or-K(2-[2-(2-amino-ethoxy)-ethoxy]-acetyl)-(γGlu)-CO-(CH₂)₁₆- |
| | CO₂H)AQ-Aib-EFIe-αMeY-LIEGSPSSGAPPPS |
| 311 | Y-Aib-EGT-αMeF(2F)-TSD-4Pal-SI-αMeL-LD-Or-K(ϵKϵK-(γGlu)-CO-(CH₂)₁₆-CO₂H)AQ-Aib-EFIe-αMeY- |
| | LVEGSPSSGAPPPS |
| 312 | Y-Aib-EGT-αMeF(2F)-TSD-4Pal-SI-αMeL-LD-Om-K(2-[2-(2-amino-ethoxy)-ethoxy]-acetyl)₂-(γGlu)-CO- |
| | (CH₂)₁₆-CO₂H)AQ-Aib-EFIE-αMeY-LIAGSPSSGAPPPS |
| 313 | Y-Aib-EGT-αMeF(2F)-TSD-4Pal-SI-αMeL-LD-Or-K(ϵKϵK-(γGlu)-CO-(CH₂)₁₆-CO₂H)AQ-Aib-EFIE-αMeY- |
| | LIAGSPSSGAPPPS |
| 314 | Y-Aib-EGT-αMeF(2F)-TSDY-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIE-αMeY-LIAGSPSSGAPPPS |
| 315 | Y-Aib-EGT-αMeF(2F)-TSDY-Aib-I-αMeL-LD-Om-K(ϵKϵK-(γGlu)-CO-(CH₂)₁₆-CO₂H)AQ-Aib-EFIE-αMeY- |
| | LIAGSPSSGAPPPS |
| 316 | Y-Aib-EGT-αMeF(2F)-TSD-4Pal-SI-αMeL-LDEK(Ac)AQ-Aib-EFIe-αMeY-LIEGSPSSGAPPPS |
| 317 | Y-Aib-EGT-αMeF(2F)-TSD-4Pal-SI-αMeL-LDEQAQ-Aib-EFIe-αMeY-LIEGSPSSGAPPPS |
| 318 | Y-Aib-EGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₈-CO₂H)AQ-Aib-EFIe-αMeY-LIEGSPSSGAPPPS |
| 319 | Y-Aib-EGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₄-CO₂H)AQ-Aib-EFIe-αMeY-LIEGSPSSGAPPPS |
| 320 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₄- |
| | CO₂H)AQ-Aib-EFIe-αMeY-LIEGSPSSGAPPPS |
| 321 | Y-Aib-EGT-αMeF(2F)-TSD-4Pal-SI-αMeL-LD-Or-K(ϵKϵK-(γGlu)-CO-(CH₂)₁₈-CO₂H)AQ-Aib-EFIe-αMeY- |
| | LIEGSPSSGAPPPS |
| 322 | Y-Aib-EGT-αMeF(2F)-TSD-4Pal-SI-αMeL-LDEK(ϵKϵK-(γGlu)-CO-(CH₂)₁₈-CO₂H)AQ-Aib-EFIe-αMeY- |
| | LIEGSPSSGAPPPS |
| 323 | Y-Aib-EGT-αMeF(2F)-TSD-4Pal-SI-αMeL-LD-Om-K(2-[2-(2-amino-ethoxy)-ethoxy]-acetyl)₂-(γGlu)-CO- |
| | (CH₂)₁₄-CO₂H)AQ-Aib-EFIE-αMeY-LIEGGG |
| 324 | Y-Aib-EGT-αMeF(2F)-TSD-4Pal-SI-αMeL-LDEK(γGlu-(2-[2-(2-amino-ethoxy)-ethoxy]-acetyl)-ϵK-CO-(CH₂)₁₈- |
| | CO₂H)AQ-Aib-EFIe-αMeY-LIEGSPSSGAPPPS |
| 325 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₄-CO₂H)AQ-Aib-EFIE-αMeY-LIEGGG |
| 326 | Y-Aib-EGT-αMeF(2F)-TSD-4Pal-Aib-I-αMeL-LD-Or-K(2-[2-(2-amino-ethoxy)-ethoxy]-acetyl)-(γGlu)-CO- |
| | (CH₂)₁₄-CO₂H)AQ-Aib-EFIE-αMeY-LIEGGPSSG |
| 327 | Y-Aib-EGT-αMeF(2F)-TSD-4Pal-Aib-I-αMeL-LDEK(ϵKϵK-(γGlu)-CO-(CH₂)₁₄-CO₂H)AQ-Aib-EFIE-αMeY- |
| | LIEGGPSSG |
| 328 | Y-Aib-EGT-aMcF(2F)-TSD-4Pal-Aib-I-αMeL-LDEK(ϵKϵK-(γGlu)-CO-(CH₂)₁₄-CO₂H)AQ-Aib-EFIE-αMeY- |
| | LIEGGG |
| 329 | Y-Aib-EGT-αMeF(2F)-TSD-4Pal-SI-αMeL-LDEK(2-[2-(2-amino-ethoxy)-ethoxy]-acetyl)₂-(γGlu)-CO-(CH₂)₁₄- |
| | CO₂H)AQ-Aib-EFIE-αMeY-LIEGGG |
| 330 | Y-Aib-EGT-αMeF(2F)-TSDY-Aib-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIe-αMeY-LIEGSPSSGAPPPS |
| 331 | Y-Aib-EGT-αMeF(2F)-TSD-4Pal-Aib-I-αMeL-LD-Or-K(2-[2-(2-amino-ethoxy)-ethoxy]-acetyl)₂-(γGlu)-CO- |
| | (CH₂)₁₆-CO₂H)AQ-Aib-EFIQ-αMeY-LIE-Aib-SPSSGAPPPS |
| 332 | Y-Aib-EGT-αMeF(2F)-TSD-4Pal-Aib-I-αMeL-LD-Orn-K(ϵKϵK-(γGlu)-CO-(CH₂)₁₆-CO₂H)AQ-Aib-EFIQ-αMeY- |
| | LIE-Aib-SPSSGAPPPS |
| 333 | Y-Aib-QGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Orn-FIe-αMeY-LIEGSPSSGAPPPS |
| 334 | Y-Aib-QGT-αMeF(2F)-TSD-4Pal-SI-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-Om-FIE-αMeY-LIEGSPSSGAPPPS |
| 335 | Y-Aib-QGT-αMeF(2F)-TSD-4Pal-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-Orn-FIe-αMeY-LIEGSPSSGAPPPS |
| 336 | Y-Aib-QGT-αMeF(2F)-TSD-4Pal-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-Om-FIE-αMeY-LIEGSPSSGAPPPS |
| 337 | Y-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Om-FIe-αMeY-LIEGSPSSGAPPPS |
| 338 | Y-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Om-FIE-αMeY-LIEGSPSSGAPPPS |
| 339 | Y-Aib-QGT-αMeF(2F)-TSD-4Pal-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Or-FIE-αMeY-LIEGGG |
| 340 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Or-FIe-αMeY-LIEGGG |
| 341 | Y-Aib-QGT-αMeF(2F)-TSD-4Pal-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Om-FIE-αMeY-LIEGGPSSGAPPPS |
| 342 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Orn-FIe-αMeY-LIEGGPSSGAPPPS |
| 343 | Y-Aib-QGT-αMeF(2F)-TSD-4Pal-αMeS-I-αMeL-LD-Om-KAQ-αMe4Pal-Orn-FIE-αMeY-LIEGGPSSGAPPPS |
| 344 | Y-Aib-QGT-αMeF(2F)-TSDY-αMeS-I-αMeL-LD-Orn-K(ϵKϵK-(γGlu)-CO-(CH₂)₁₆-CO₂H)AQ-αMe4Pal-Om-FIe- |
| | αMeY-LIEGGPSSGAPPPS |
| 345 | Y-Aib-QGT-αMeF(2F)-TSD-4Pal-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-Orn-FIK-αMeY-LIEGGPSSGAPPPSGG |
| 346 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Om-FIK-αMeY-LIEGGPSSGAPPPSGG |
| 347 | Y-Aib-QGT-αMeF(2F)-TSD-4Pal-αMeS-I-αMeL-LD-Or-K(ϵKϵK-(γGlu)-CO-(CH₂)₁₆-CO₂H)AQ-αMe4Pal-Om- |
| | FIK-αMeY-LIEGGPSSGAPPPSGG |
| 348 | Y-Aib-QGT-αMeF(2F)-TSDY-αMeS-I-αMeL-LD-Orn-K(ϵKϵK-(γGlu)-CO-(CH₂)₁₆-CO₂H)AQ-αMe4Pal-Or- |
| | FIKαMeY-LIEGGPSSGAPPPSGG |
| 349 | Y-Aib-QGT-αMeF(2F)-TSD-4Pal-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-Om-FIE-αMeY-LIEGGPSSG |
| 350 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Or-FIe-αMeY-LIEGGPSSG |
| 351 | Y-Aib-QGT-αMeF(2F)-TSD-4Pal-αMeS-I-αMeL-LD-Or-K(ϵKϵK-(γGlu)-CO-(CH₂)₁₆-CO₂H)AQ-αMe4Pal-Orn- |
| | FIE-αMeY-LIEGGPSSG |
| 352 | Y-Aib-QGT-αMeF(2F)-TSDY-αMeS-I-αMeL-LD-Orn-K(ϵKϵK-(γGlu)-CO-(CH₂)₁₆-CO₂H)AQ-αMe4Pal-Om-FIe- |
| | αMeY-LIEGGPSSG |
| 353 | Y-Aib-QGT-aMcF(2F)-TSD-4Pal-αMeS-I-αMeL-LD-Om-K(ϵKϵK-(γGlu)-CO-(CH₂)₁₆-CO₂H)AQ-αMe4Pal-Orn- |
| | FIE-αMeY-LIEGGG |
| 354 | Y-Aib-QGT-αMeF(2F)-TSDY-αMeS-I-αMeL-LD-Orn-K(ϵKϵK-(γGlu)-CO-(CH₂)₁₆-CO₂H)AQ-αMe4Pal-Om-FIe- |
| | αMeY-LIEGGG |
| 355 | Y-Aib-QGT-αMeF(2F)-TSD-4Pal-αMeS-I-αMeL-LD-Om-K(ϵKϵK-(γGlu)-CO-(CH₂)₁₆-CO₂H)AQ-αMe4Pal-Om- |
| | FIE-αMeY-LIEGSPSSGAPPPS |
| 356 | Y-Aib-QGT-αMeF(2F)-TSDY-αMeS-I-αMeL-LD-Orn-K(ϵKϵK-(γGlu)-CO-(CH₂)₁₆-CO₂H)AQ-αMe4Pal-Om-FIe- |
| | αMeY-LIEGSPSSGAPPPS |
| 357 | Y-Aib-QGT-αMeF(2F)-TSD-4Pal-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Om-FIe-αMeY-LIEGSPSSG |
| 358 | Y-Aib-QGT-αMeF(2F)-TSD-4Pal-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-Orn-FIe-αMeY-LIEGGG |
| 359 | Y-Aib-QGT-αMeF(2F)-TSD-4Pal-αMeS-I-αMeL-LD-Orn-K(ϵKϵK-(γGlu)-CO-(CH₂)₁₆-CO₂H)AQ-αMe4Pal-Om- |
| | FIe-αMeY-LIEGSPSSGAPPPS |
| 360 | Y-Aib-QGT-αMeF(2F)-TSD-4Pal-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Orn-FIQ-αMeY-LIEGSPSSGAPPPS |
| 361 | Y-Aib-QGT-αMeF(2F)-TSD-4Pal-αMeS-I-αMeL-LD-Om-K(ϵKϵK-(γGlu)-CO-(CH₂)₁₆-CO₂H)AQ-αMe4Pal-Om- |
| | FIQ-αMeY-LIEGSPSSGAPPPS |
| 362 | Y-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Orn-FIQ-αMeY-LIEGSPSSGAPPPS |
| 363 | Y-Aib-QGT-αMeF(2F)-TSDY-αMeS-I-αMeL-LD-Orn-K(ϵKϵK-(γGlu)-CO-(CH₂)₁₆-CO₂H)AQ-αMe4Pal-Or-FIQ- |
| | αMeY-LIEGSPSSGAPPPS |
| 364 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Orn-FIe-αMeY-LIE-Aib-SPSSGAPPPS |
| 365 | Y-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-EFIe-αMeY-LIEGSPSSGAPPPS |
| 366 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 367 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂) 16-CO₂H)AQ-Aib-EFIQ-αMeY-LIEGSPSSGAPPPS |
| 368 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LLEGSPSSGAPPPS |
| 369 | Y-Aib-QGT-αMeF(2F)-TSD-4Pal-SI-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Or-FIQ-αMeY-LIEGSPSSGAPPPS |
| 370 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Or-FIe-αMeY-LIE-Aib-GG |
| 371 | Y-Aib-QaT-aMcF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 372 | Y-αMeS-QGT-αMeF(2F)-TSDY-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 373 | Y-Iva-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 374 | Y-Aib-HGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 375 | H-Aib-HGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 376 | H-Aib-HGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIQ-αMeY-LIEGSPSSGAPPPS |
| 377 | H-Aib-HGT-αMeF(2F)-TSD-4Pal-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 378 | H-Aib-HGT-αMeF(2F)-TSD-4Pal-SI-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 379 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIE-Aib-SPSSGAPPPS |
| 380 | Y-Aib-QGT-αMeF(2F)-TSDY-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LIEGSPSSGAPPPS |
| 381 | Y-Aib-QGT-αMeF(2F)-TSD-4Pal-αMeS-I-αMeL-LD-Om-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 382 | Y-Aib-QGT-αMeF(2F)-TSD-4Pal-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LIEGSPSSGAPPPS |
| 383 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIQ-αMeY-LIEGSPSSGAPPPS |
| 384 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LIE-Aib-SPSSGAPPPS |
| 385 | Y-Aib-QGT-αMeF(2F)-TSD-4Pal-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIE-Aib-SPSSGAPPPS |
| 386 | Y-Aib-QGT-αMeF(2F)-TSD-4Pal-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LIE-Aib-SPSSGAPPPS |
| 387 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIQ-αMeY-LIE-Aib-SPSSGAPPPS |
| 388 | Y-Aib-QGT-αMeF(2F)-TSDY-αMeS-I-αMeL-LDEK(2-[2-(2-amino-ethoxy)-ethoxy]-acetyl)-(γGlu)-CO-(CH₂)₁₆- |
| | CO₂H)AQ-αMe4Pal-EFIQ-αMeY-LLEGSPSSGAPPPS |
| 389 | Y-Aib-QGT-αMeF(2F)-TSDY-αMeS-I-αMeL-LDEK(2-[2-(2-amino-ethoxy)-ethoxy]-acetyl)-(γGlu)-CO-(CH₂)₁₆- |
| | CO₂H)AQ-αMe4Pal-EFIQ-αMeY-LLE-Aib-SPSSGAPPPS |
| 390 | H-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGSPSSGAPPPS |
| 391 | Y-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-L VEGSPSSGAPPPS |
| 392 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LIEGGG |
| 393 | H-Aib-HGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-Aib-EFIQ-αMeY-LLEGSPSSGAPPPS |
| 394 | H-Aib-HGT-αMeF(2F)-TSD-4Pal-SI-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LLEGSPSSGAPPPS |
| 395 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIE-αMeY-LIEGSPSSGAPPPS |
| 396 | Y-Aib-QGT-αMeF(2F)-TSD-4Pal-αMeS-I-αMeL-LD-Om-K(2-[2-(2-amino-ethoxy)-ethoxy]-acetyl)-(γGlu)-CO- |
| | (CH₂)₁₆-CO₂H)AQ-αMe4Pal-EFIQ-αMeY-LIEGSPSSGAPPPS |
| 397 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-DFIQ-αMeY-LIEGSPSSGAPPPS |
| 398 | Y-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₆-CO₂H)AQ-αMe4Pal-Orn-FIE-αMeY-LIA-Aib-SPSSGAPPPS |
| 399 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LLE-Aib-SPSSGAPPPS |
| 400 | H-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LIEGSPSSGAPPPS |
| 401 | H-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LIE-Aib-SPSSGAPPPS |
| 402 | H-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LLEGSPSSGAPPPS |
| 403 | H-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LLE-Aib-SPSSGAPPPS |
| 404 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIE-αMeY-LIEGSPSSGAPPPS |
| 405 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIE-αMeY-LIE-Aib-SPSSGAPPPS |
| 406 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIE-αMeY-LLEGSPSSGAPPPS |
| 407 | H-Aib-QGT-αMeF(2F)-TSDY-αMeS-I-αMeL-LD-Orn-K(2-12-(2-amino-ethoxy)-ethoxy]-acetyl)₂-(γGlu)-CO- |
| | (CH₂)₁₆-CO₂H)AQ-αMe4Pal-EFIQ-αMeY-LIE-Aib-SPSSGAPPPS |
| 408 | H-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIQ-αMeY-LLE-Aib-SPSSGAPPPS |
| 409 | H-Aib-QGT-αMeF(2F)-TSDY-αMeS-I-αMeL-LD-Or-K(2-[2-(2-amino-ethoxy)-ethoxy]-acetyl)-(γGlu)-CO- |
| | (CH₂)₁₆-CO₂H)AQ-αMe4Pal-EFIQ-αMeY-LIE-Aib-SPSSGAPPPS |
| 410 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIQ-αMeY-LLE-Aib-SPSSGAPPPS |
| 411 | Y-αMeS-QGT-αMeF(2F)-TSDY-αMeS-I-αMeL-LD-Or-K(2-[2-(2-amino-ethoxy)-ethoxy]-acetyl)-(γGlu)-CO- |
| | (CH₂)₁₆-CO₂H)AQ-αMe4Pal-EFIE-αMeY-LIEGSPSSGAPPPS |
| $12 | Y-Iva-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIE-αMeY-LIEGSPSSGAPPPS |
| 413 | Y-Ac4c-QGT-αMeF(2F)-TSDY-αMeS-I-αMeL-LD-Or-K(2-[2-(2-amino-ethoxy)-ethoxy]-acetyl)-(γGlu)-CO- |
| | (CH₂)₁₆-CO₂H)AQ-αMe4Pal-EFIE-αMeY-LIEGSPSSGAPPPS |
| 414 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Orn-FIK-αMeY-LLEGSPSSGAPPPS |
| 415 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIKαMeY-LLEGSPSSGAPPPS |
| 416 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Orn-FIe-αMeY-LIEGSPSSGAPPPS |
| 417 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Om-FIe-αMeY-LLEGSPSSGAPPPS |
| 418 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Or-FIE-αMeY-LIEGSPSSGAPPPS |
| 419 | H-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Or-FIE-αMeY-LLEGSPSSGAPPPS |
| 420 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIE-αMeY-LLEGSPSSGAPPPS |
| 421 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIe-αMeY-LLEGSPSSGAPPPS |
| 422 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Om-FIE-αMeY-LLEGSPSSGAPPPS |
| 423 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Orn-FIe-αMeY-LLEGSPSSGAPPPS |
| 424 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-EFIe-αMeY-LIEGSPSSGAPPPS |
| 425 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Orn-FIE-αMeY-LIEGSPSSGAPPPS |
| 426 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-αMe4Pal-Orn-FIe-αMeY-LIEGSPSSGAPPPS |
| 427 | Y-Aib-QGT-αMeF(2F)-TSDY-αMeS-I-αMeL-LD-Or-K(2-[2-(2-amino-ethoxy)-ethoxy]-acetyl)-(γGlu)-CO- |
| | (CH₂)₁₆-CO₂H)AQ-Aib-QFIE-αMeY-LLEGSPSSGAPPPS |
| 428 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-Orn-FIe-αMeY-LLEGSPSSGAPPPS |
| 429 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-Orn-FIE-αMeY-LLEGSPSSGAPPPS |
| 430 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-Orn-FIq-αMeY-LLEGSPSSGAPPPS |
| 431 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-Orn-FIQ-αMeY-LLEGSPSSGAPPPS |
| 432 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-EFIe-αMeY-LLEGSPSSGAPPPS |
| 433 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-EFIE-αMeY-LLEGSPSSGAPPPS |
| 434 | Y-Aib-QGT-αMeF(2F)-TSDY-αMeS-I-αMeL-LD-Or-K(2-[2-(2-amino-ethoxy)-ethoxy]-acetyl)-(γGlu)-CO- |
| | (CH₂)₁₆-CO₂H)AQ-Aib-EFIq-αMeY-LLEGSPSSGAPPPS |
| 435 | H-Ac4c-QGT-αMeF(2F)-TSDY-αMeS-I-αMeL-LD-Om-K(2-[2-(2-amino-ethoxy)-ethoxy]-acetyl)-(γGlu)-CO- |
| | (CH₂)₁₆-CO₂H)AQ-αMe4Pal-EFIE-αMeY-LIEGSPSSGAPPPS |
| 436 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIEGSPSSGAPPPS |
| 437 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Or-FIE-αMeY-LIEGSPSSGAPPPS |
| 438 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIe-αMeY-LIEGSPSSGAPPPS |
| 439 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LLEGSPSSGAPPPS |
| 440 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIe-αMeY-LLEGSPSSGAPPPS |
| 441 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-αMeY-LLEGSPSSGAPPPS |
| 442 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-Aib-Orn-Flq-αMeY-LLEGSPSSGAPPPS |
| 443 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-Aib-Om-FIQ-αMeY-LLEGSPSSGAPPPS |
| 444 | YaQGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-Aib-Orn-FIE-αMeY-LIEGSPSSGAPPPS |
| 445 | Y-Aib-QGT-αMeF(2F)-TSD-4Pal-SI-αMeL-LD-Om-K(2-[2-(2-amino-ethoxy)-ethoxy]-acetyl)-(γGlu)-CO-(CH₂)₁₆- |
| | CO₂H)AQ-αMe4Pal-Orn-FIE-αMeY-LIEGSPSSGAPPPS |
| 446 | Y-Aib-QGT-αMeF(2F)-TSD-4Pal-αMeS-I-αMeL-LD-Om-K(2-[2-(2-amino-ethoxy)-ethoxy]-acetyl)-(γGlu)-CO- |
| | (CH₂)₁₆-CO₂H)AQ-αMe4Pal-Om-FIe-αMeY-LIEGSPSSGAPPPS |
| 447 | Y-Aib-QGT-αMeF(2F)-TSD-4Pal-αMeS-I-αMeL-LD-Or-K(2-[2-(2-amino-ethoxy)-ethoxy]-acetyl)-(γGlu)-CO- |
| | (CH₂)₁₆-CO₂H)AQ-αMe4Pal-EFIE-αMeY-LIEGSPSSGAPPPS |
| 448 | Y-Aib-QGT-αMeF(2F)-TSD-4Pal-SI-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIEGSPSSGAPPPS |
| 449 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-Orn-FIE-αMeY-LIEGSPSSGAPPPS |
| 450 | Y-Aib-QGT-αMeF(2F)-TSD-4Pal-αMeS-I-αMeL-LD-Om-K(2-[2-(2-amino-ethoxy)-ethoxy]-acetyl)-(γGlu)-CO- |
| | (CH₂)₁₆-CO₂H)AQ-αMe4Pal-EFIe-αMeY-LIEGSPSSGAPPPS |
| 451 | YaQGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-Orn-FIE-αMeY-LIEGSPSSGAPPPS |
| 452 | YaQGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-Orn-FIE-αMeY-LLEGSPSSGAPPPS |
| 453 | Y-Aib-QGT-αMeF(2F)-TSDY-αMeS-I-αMeL-LD-Orn-K(2-[2-(2-amino-ethoxy)-ethoxy]-acetyl)-(γGlu)-CO- |
| | (CH₂)₁₆-CO₂H)AQ-Aib-Orn-FIe-αMeY-LIEGSPSSGAPPPS |
| 454 | NMeY-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-EFIE-αMeY-LIEGSPSSGAPPPS |
| 455 | Y-Aib-QGT-αMeF(2F)-TSDY-αMeS-I-αMeL-LD-Or-K(2-[2-(2-amino-ethoxy)-ethoxy]-acetyl)₂-(γGlu)-CO- |
| | (CH₂)₁₈-CO₂H)AQ-αMe4Pal-Orn-FIe-αMeY-LIEGSPSSGAPPPS |
| 456 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₈-CO₂H)AQ-αMe4Pal-Or-FIQ-αMeY-LIEGSPSSGAPPPS |
| 457 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Om-FIE-αMeY-LIE-Aib-SPSSGAPPPS |
| 458 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO- |
| | (CH₂)₁₆-CO₂H)AQ-αMe4Pal-Or-FIQ-αMeY-LIE-Aib-SPSSGAPPPS |
| 1039 | Y-Aib-EGT-αMeF(2F)-TSD-Y-Aib-I-αMeL-LD-Orn-K(2-[2-(2-amino-ethoxy)-ethoxy]-acetyl)₂-(γGlu)-CO-(CH₂)₁₆- |
| | CO₂H)AQ-Aib-Orn-FIE-αMeY-LIEGSPSSGAPPPS |
| 1040 | Y-Aib-EGT-αMeF(2F)-TSDY-Aib-I-αMeL-LD-Orn-K(2-[2-(2-amino-ethoxy)-ethoxy]-acetyl)₂-(γGlu)-CO-(CH₂)₁₆- |
| | CO₂H)AQ-Aib-EFIE-αMeY-LIAGGG |
| 1041 | Y-Aib-EGT-αMeF(2F)-TSDY-Aib-I-αMeL-LD-Orn-K(2-[2-(2-amino-ethoxy)-ethoxy]-acetyl)₂-(γGlu)-CO-(CH₂)₁₆- |
| | CO₂H)AQ-Aib-EFIE-αMeY-LIE-Aib-GG |
| 1042 | Y-Aib-EGT-αMeF(2F)-TSDY-Aib-I-αMeL-LD-Orn-K(2-[2-(2-amino-ethoxy)-ethoxy]-acetyl)₂-(γGlu)-CO-(CH₂)₁₄- |
| | CO₂H)AQ-Aib-EFIE-αMeY-LIEGSPSSGAPPPS |
| 1043 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-Orn-FIE-αMeY-L VEGSPSSGAPPPS |
| 1044 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-αMeY-LIEGSPSSGAPPPS |
| 1045 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-αMeY-L VEGSPSSGAPPPS |
| 1046 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-αMeY-L VEGSPSSGAPPPS |
| 1047 | Y-Aib-QGT-αMeF(2F)-TSD-4Pal-SI-αMeL-LD-Or-K(ϵKϵK-(γGlu)-CO-(CH₂)₁₆-CO₂H)AQ-αMe4Pal-Orn-FIE-αMeY- |
| | LIEGSPSSGAPPPS |
| 1048 | Y-Aib-QGT-aMcF(2F)-TSDY-αMeS-I-αMeL-LD-Orn-K(ϵKϵK-(γGlu)-CO-(CH₂)₁₆-CO>H)AQ-αMe4Pal-Orn-FIE-αMe Y- |
| | LIEGSPSSGAPPPS |
| 1049 | Y-Aib-QGT-αMeF(2F)-TSD-4Pal-αMeS-I-αMeL-LD-Orn-K(ϵKϵK-(γGlu)-CO-(CH₂)₁₆-CO₂H)AQ-αMe4Pal-EFIE-αMeY- |
| | LIEGSPSSGAPPPS |
| 1050 | Y-Aib-QGT-αMeF(2F)-TSDY-αMeS-I-αMeL-LD-Or-K(ϵKϵK-(γGlu)-CO-(CH₂)₁₆-CO₂H)AQ-αMe4Pal-EFIE-αMe Y- |
| | LIEGSPSSGAPPPS |
| 1051 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LVEGSPSSGAPPPS |
| 1052 | YaQGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-QMeY-LIEGSPSSGAPPPS |
| 1053 | YaQGT-αMeF(2F)-TSD-4Pal-SI-αMeL-LD-Orn-K(2-[2-(2-amino-ethoxy)-ethoxy]-acetyl)-(γGlu)-CO-(CH₂)₁₆-CO₂H)AQ- |
| | αMe4Pal-Orn-FIE-αMeY-LIEGSPSSGAPPPS |
| 1054 | YaQGT-αMeF(2F)-TSDY-αMeS-I-αMeL-LD-Orn-K(2-[2-(2-amino-ethoxy)-ethoxy]-acety1)-(γGlu)-CO-(CH₂)₁₆-CO₂H)AQ- |
| | αMe4Pal-Or-FIE-αMe Y-LIEQSPSSGAPPPS |
| 1055 | Y-αMeS-QGT-αMeF(2F)-TSDY-αMeS-I-αMeL-LD-Orn-K(2-[2-(2-amino-ethoxy)-ethoxy]-acety1)-(γGlu)-CO-(CH₂)₁₆- |
| | CO₂H)AQ-αMe4Pal-Orn-FIE-αMeY-LIEGSPSSGAPPPS |
| 1056 | Y-αMeS-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-QMeY-LIEGSPSSGAPPPS |
| 1057 | Y-αMeS-QGT-αMeF(2F)-TSDY-αMeS-I-αMeL-LD-Or-K(2-[2-(2-amino-ethoxy)-ethoxy]-acetyl)₂-(γGlu)-CO-(CH₂)₁₆- |
| | CO₂H)AQ-αMe4Pal-Orn-FIE-αMeY-LIEGSPSSGAPPPS |
| 1058 | Y-Iva-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-αMeY-LIEGSPSSGAPPPS |
| 1059 | Y-Iva-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-Orn-FIE-αMeY-LIEGSPSSGAPPPS |
| 1060 | Y-Iva-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-Orn-FIE-αMeY-LIEGSPSSGAPPPS |
| 1061 | Y-Ac4c-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIEGSPSSGAPPPS |
| 1062 | Y-Ac4c-QGT-αMeF(2F)-TSDY-αMeS-I-αMeL-LD-Om-K(2-[2-(2-amino-ethoxy)-ethoxy]-acetyl)₂-(γGlu)-CO-(CH₂)₁₆- |
| | CO₂H)AQ-αMe4Pal-EFIE-αMeY-LIEGSPSSGAPPPS |
| 1063 | Y-Aib-QGT-αMeF(2F)-TSDY-αMeS-I-αMeL-LD-Orn-K(ϵKϵK-(γGlu)-CO-(CH₂)₁₆-CO₂H)AQ-αMe4Pal-EFIe-αMeY- |
| | LIEGSPSSGAPPPS |
| 1064 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMeL-Orn-FIe-αMeY-LIEGSPSSGAPPPS |
| 1065 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMeL-Iva-FIe-αMeY-LIEGSPSSGAPPPS |
| 1066 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMeL-Orn-FIe-αMeY-LIEGSPSSGAPPPS |
| 1067 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMeL-Iva-FIe-αMeY-LIEGSPSSGAPPPS |
| 1068 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-EFIE-MeY-LIAGSPSSGAPPPS |
| 1069 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIAGSPSSGAPPPS |
| 1070 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-EFIE-αMeY-LIEaSPSSGAPPPS |
| 1071 | Y-Aib-QGT-aMcF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIe-αMeY-LIAGSPSSGAPPPS |
| 1072 | YaQGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-Orn-FIE-αMeY-LIAGSPSSGAPPPS |
| 1073 | Y-Aib-QGT-αMeF(2F)-TSDY-αMeS-I-αMeL-LD-Orn-K(2-[2-(2-amino-ethoxy)-ethoxy]-acety1)-(γGlu)-CO-(CH₂)₁₆- |
| | CO₂H)AQ-Aib-Orn-FIe-αMeY-LIAGSPSSGAPPPS |
| 1074 | YaQGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-Orn-FIE-αMeY-LIEaSPSSGAPPPS |
| 1075 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-SFIE-αMeY-LIEGSPSSGAPPPS |
| 1076 | Y-Aib-QGT-aMcF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-Aib-SFIE-αMeY-LIEGSPSSGAPPPS |
| 1077 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-4Pal-AFIE-αMeY-LIEGSPSSGAPPPS |
| 1078 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMeF-Aib-FIe-αMeY-LIEGSPSSGAPPPS |
| 1079 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-Iva-AFIe-αMeY-LIEGSPSSGAPPPS |
| 1080 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-Aib-EFIE-αMeY-LIEGSPSSGAPPPS |
| 1081 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIe-αMeY-LIE-Aib-SPSSGAPPPS |
| 1082 | Y-Aib-QGT-αMeF(2F)-TSDY-αMeS-I-αMeL-LD-Or-K(2-[2-(2-amino-ethoxy)-ethoxy]-acetyl)-(γGlu)-CO-(CH₂)₁₆- |
| | CO₂H)AQ-αMeL-Orn-FIe-αMeY-LIAaSPSSGAPPPS |
| 1083 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIE-Aib-SPSSGAPPPS |
| 1084 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIAGSPSSGAPPPS |
| 1085 | Y-Aib-QGT-αMeF(2F)-TSD-4Pal-αMeS-I-αMeL-LD-Orn-K(2-[2-(2-amino-ethoxy)-ethoxy]-acety1)-(γGlu)-CO-(CH₂)₁₆- |
| | CO₂H)AQ-αMe4Pal-EFIE-αMeY-LIAaSPSSGAPPPS |
| 1086 | Y-Aib-QGT-αMeF(2F)-TSD-4Pal-αMeS-I-αMeL-LD-Orn-K(2-[2-(2-amino-ethoxy)-ethoxy ]-acetyl)-(γGlu)-CO-(CH₂)₁₆- |
| | CO₂H)AQ-αMeL-Iva-FIe-αMeY-LIA-Aib-SPSSGAPPPS |
| 1087 | Y-Aib-QGT-αMeF(2F)-TSD-4Pal-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-SFIE-αMeY-LIEGSPSSGAPPPS |
| 1088 | Y-Aib-QGT-αMeF(2F)-TSD-4Pal-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-SFIE-αMeY-LIE-Aib-SPSSGAPPPS |
| 1089 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-EFIE-αMeY-LIEGGG |
| 1090 | Y-Aib-QGT-αMeF(2F)-TSDY-αMeS-I-αMeL-LD-Orn-K(2-[2-(2-amino-ethoxy)-ethoxy]-acetyl)₂-(γGlu)-CO-(CH₂)₁₆- |
| | CO₂H)AQ-αMe4Pal-EFIE-αMeY-LIAGGG |
| 1091 | Y-Aib-QGT-αMeF(2F)-TSDY-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIEGGG |
| 1092 | Y-Aib-QGT-αMeF(2F)-TSDY-αMeS-I-αMeL-LD-Orn-K(2-[2-(2-amino-ethoxy)-ethoxy]-acety1)-(γGlu)-CO-(CH₂)₁₆- |
| | CO₂H)AQ-αMe4Pal-EFIe-αMeY-LIEGGG |
| 1093 | Y-Ac4c-QGT-αMeF(2F)-TSDY-αMeS-I-αMeL-LD-Om-K(2-[2-(2-amino-ethoxy)-ethoxy]-acetyl)-(γGlu)-CO-(CH₂)₁₆- |
| | CO₂H)AQ-αMe4Pal-EFIE-αMeY-LIEGGG |
| 1094 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₆- |
| | CO₂H)AQ-αMe4Pal-EFIE-αMeY-LIEGGG |
| 1095 | Y-Aib-QGT-αMeF(2F)-TSDY-αMeS-I-QMeL-LD-Orn-K(2-[2-(2-amino-ethoxy)-ethoxy]-acetyl)₂-(γGlu)-CO-(CH₂)₁₄- |
| | CO₂H)AQ-αMe4Pal-EFIE-αMeY-LIEGSPSSGAPPPS |
| 1096 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₄- |
| | CO₂H)AQ-αMe4Pal-EFIE-αMeY-LIEGSPSSGAPPPS |
| 1097 | Y-Aib-QGT-αMeF(2F)-TSDY-αMeS-I-αMeL-LD-Or-K(2-[2-(2-amino-ethoxy)-ethoxy]-acetyl)-(γGlu)-CO-(CH₂)₁₄- |
| | CO₂H)AQ-αMe4Pal-EFIE-αMeY-LIEGSPSSGAPPPS |
| 1098 | Y-Aib-QGT-αMeF(2F)-TSD-4Pal-αMeS-I-αMeL-LD-Orn-K(2-[2-(2-amino-ethoxy)-ethoxy]-acetyl)-(γGlu)-CO-(CH₂)₁₄- |
| | CO₂H)AQ-αMe4Pal-EFIe-αMeY-LIEGSPSSGAPPPS |

### Other Definitions and Abbreviations

The amino acid sequences of the therapeutic polypeptides described herein incorporate naturally occurring amino acids, typically depicted herein using standard one letter codes (e.g., L=leucine), as well as alpha-methyl substituted residues of natural amino acids (e.g., α-methyl leucine (αMeL), and certain other non-natural amino acids, such as alpha amino isobutyric acid (Aib). The structures of these amino acids are depicted below:

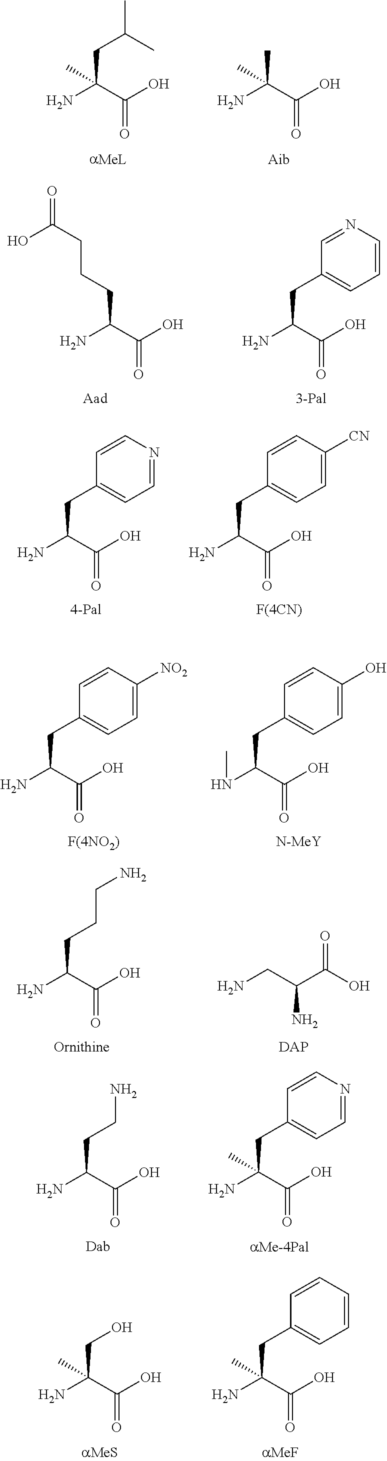

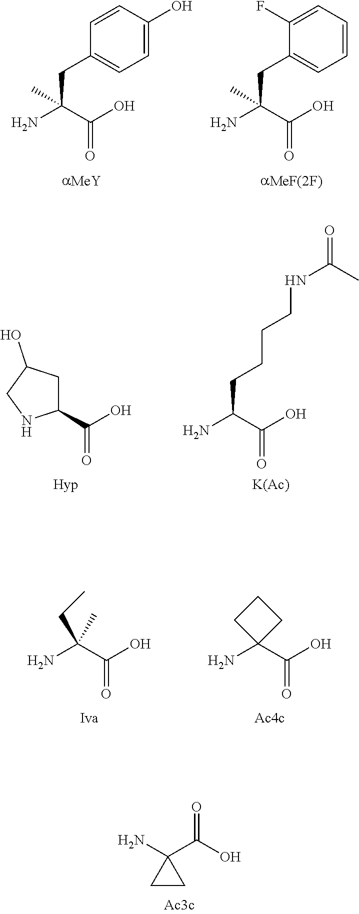

As used herein “Orn” means L-ornithine. As used herein “4-Pal” or “4Pal” means 3-(4-Pyridyl)-L-alanine or (S)-2-amino-3-(pyridin-4-yl)propanoic acid. As used herein “3-Pal” or “3Pal” means 3-(3-Pyridyl)-L-alanine or (S)-2-amino-3-(pyridin-3-yl)propanoic acid. As used herein “αMe-4-Pal” or “αMe4Pal” means alpha-methyl-3-(4-Pyridyl)-L-alanine. As used herein “αMeY” means alpha-methyl-L-tyrosine. As used herein “αMeL” means alpha-methyl-leucine. As used herein, “Ac3c” means 1-aminocyclopropanecarboxylic acid. As used herein, “Ac4c” means 1-Aminocyclobutane-1-carboxylic acid. As used herein “D-Ala” and “a” each means D-alanine. As used herein “D-Glu” and “e” each means D-glutamic acid. As used herein “Aib” means 2-Aminoisobutyric Acid. As used herein, “NMeY” means N-methyl-tyrosine. As used herein “Dap” means (S)-2,3-diaminopropanoic acid. As used herein “Dab” means (S)-2,4-diaminobutanoic acid. As used herein “Hyp” means Hydroxy-L-proline. As used herein “K(Ac)” means N⁶-acetyl-L-lysine. As used herein “γGlu” means gamma L-glutamic acid. As used herein “Aad” means (S)-2-aminohexanedioic acid. As used herein “F(4CN)” means 4-cyano-L-phenylalanine or (S)-2-amino-3-(4-cyanophenyl)propanoic acid. As used herein “F(4NO₂)” means 4-nitro-L-phenylalanine or (S)-2-amino-3-(4-nitrophenyl)propanoic acid. As used herein “αMeS” means alpha-methyl-L-serine. As used herein “αMeF” means alpha-methyl-L-phenylalanine. As used herein “αMeF(2F)” means alpha-methyl-2-fluoro-L-phenylalanine or (S)-2-amino-3-(2-fluorophenyl)-2-methylpropanoic acid. As used herein “L-Iva” and “Iva” mean L-isovaline. As used herein “D-Gln” and “q” each means D-glutamine.

Certain abbreviations used herein are defined as follows: “AcOH” refers to acetic acid; “ACN” refers to acetonitrile; “BEA” refers to 4-(bis(2-((((9H-fluoren-9-yl)methoxy)carbonyl)amino)ethyl)amino)-4-oxobutanoic acid; “Boc” refers to tert-butoxycarbonyl; “t-Bu” refers to tert-butyl; “DABA” refers to 3,5-diaminobenzoic acid; “DCM” refers to dichloromethane; ‘DMAP” refers to 4-dimethylaminopyridine; “DIC” refers to diisopropylcarbodiimide; “Et2O” refers to diethyl ether; “DIEA” refers to diisopropylethylamine; “DMF” refers to dimethylformamide; “DMAP” refers to 4-dimethylaminopyridine; “ivDde” refers to 1-(4,4-dimethyl-2,6-dioxocyclohex-1-ylidene)-3-methylbutyl; “EtOH” refers to ethanol; “EtOAc” refers to ethyl acetate; “eq” refers to equivalents; “Fmoc” refers to fluorenylmethyloxycarbonyl; “FPLC” refers to fast protein liquid chromatography; “HFIP” refers to hexafluoroisopropanol; “IPA” refers to isopropyl alcohol; “MalDab” refers to (S)-2-((tert-butoxycarbonyl)amino)-4-(2,5-dioxo-2,5-dihydro-1H-pyrrol-1-yl)butanoic acid; “MalDap” refers to (S)-3-((tert-butoxycarbonyl)amino)-2-(2,5-dioxo-2,5-dihydro-1H-pyrrol-1-yl)propanoic acid; Beta(β)-MalDap refers to (S)-2-((tert-butoxycarbonyl)amino)-3-(2,5-dioxo-2,5-dihydro-1H-pyrrol-1-yl)propanoic acid; “MeOH” refers to methanol; “Min” refers to minute/minutes; “MTT” refers to methylthiazole tetrazolium; “Mtt” refers to 4-methyltrityl; “Oxyma” refers to ethyl cyanohydroxyiminoacetate; “PEG” refers to polyethylene glycol; “PyBOP” refers to benzotriazole-1-yl-oxy-tris-pyrrolidino-phosphonium hexafluorophosphate; “RT” refers to room temperature: “t-Bu” refers to tertiary butyl; “SPPS” refers to solid-phase peptide synthesis; “soln” refers to solution; “TFA” refers to trifluoracetic acid; “TIPS” refers to triisopropylsilane; “TCEP” refers to tris(2-carboxyethyl)phosphine hydrochloride; and “THF” refers to tetrahydrofuran.

As used herein, “BEA-MalDap₂” refers to the structure:

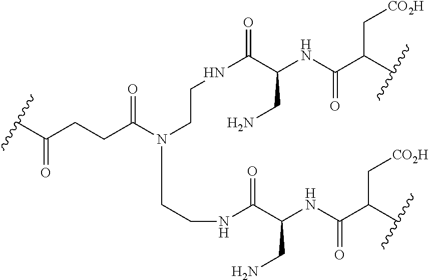 [SMILES: *C(=O)CCC(=O)N(CCNC(=O)[C@H](CN)NC(=O)C(*)CC(=O)O)CCNC(=O)[C@H](CN)NC(=O)C(*)CC(=O)O]

As used herein, “dCAP” refers to the structure:

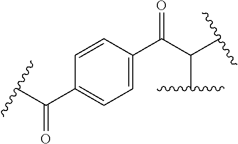 [SMILES: *C(=O)c1ccc(C(=O)C(*)*)cc1]

As used herein, “BEA-MSTP₂” refers to the structure:

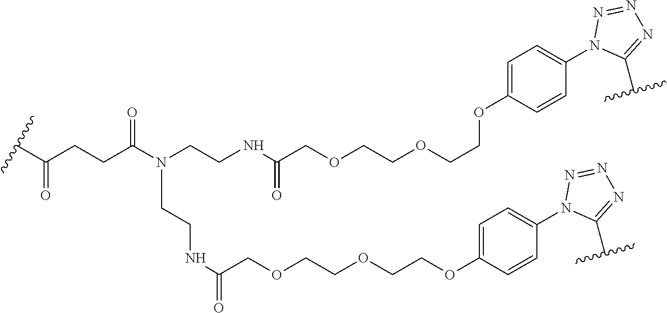 [SMILES: *C(=O)CCC(=O)N(CCNC(=O)COCCOCCOc1ccc(-n2nnnc2*)cc1)CCNC(=O)COCCOCCOc1ccc(-n2nnnc2*)cc1]

As used herein, “BEA-Acetyl₂” refers to the structure:

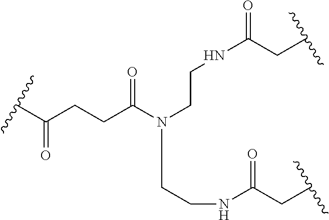 [SMILES: *CC(=O)NCCN(CCNC(=O)C*)C(=O)CCC(*)=O]

As used herein, “BEA-OD₂” refers to the structure:

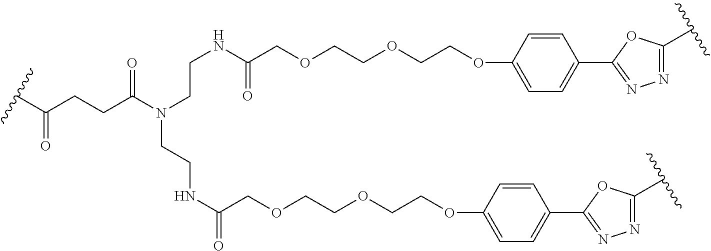 [SMILES: *C(=O)CCC(=O)N(CCNC(=O)COCCOCCOc1ccc(-c2nnc(*)o2)cc1)CCNC(=O)COCCOCCOc1ccc(-c2nnc(*)o2)cc1]

As used herein, “BEA-β-MalDap2” refers to the structure:

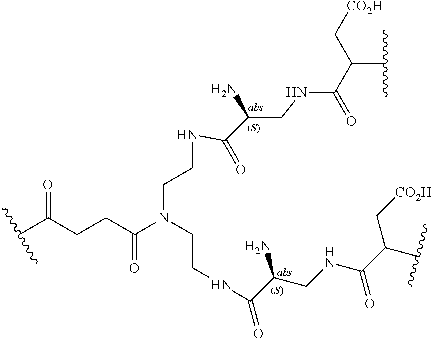

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and in reference to one or more of the GIP, GLP-1 or glucagon receptors, “activity,” “activate,” “activating” and the like means a capacity of a compound, such as the polypeptides described herein, to bind to and induce a response at the receptor(s), as measured using assays known in the art, such as the in vitro assays described below.

As used herein, “amino acid with a functional group available for conjugation” means any natural (coded) or non-natural (non-coded) amino acid with a functional group that may be conjugated to fatty acid directly or by way of, for example, a linker. Examples of such functional groups include, but are not limited to, alkynyl, alkenyl, amino, azido, bromo, carboxyl, chloro, iodo, and thiol groups. Examples of natural amino acids including such functional groups include K (amino), C (thiol), E (carboxyl) and D (carboxyl).

As used herein, “conservative amino acid substitution” means substitution of an amino acid with an amino acid having similar characteristics (e.g., charge, side-chain size, hydrophobicity/hydrophilicity, backbone conformation and rigidity, etc.) and having minimal impact on the biological activity of the resulting substituted peptide or polypeptide. Conservative substitutions of functionally similar amino acids are well known in the art and thus need not be exhaustively described herein.

As used herein, “C₁₆-C₂₂ fatty acid” means a carboxylic acid having between 16 and 22 carbon atoms. The C₁₆-C₂₂ fatty acid suitable for use herein can be a saturated monoacid or a saturated diacid. As used herein, “saturated” means the fatty acid contains no carbon-carbon double or triple bonds. Unless otherwise specified, when referred to herein, a fatty acid may also include other acidic groups, such as a phosphonic acid or a sulfonic acid.

As used herein, “effective amount” means an amount, concentration or dose of one or more polypeptides described herein, or a pharmaceutically acceptable salt thereof which, upon single or multiple dose administration to an individual in need thereof, provides a desired effect in such an individual under diagnosis or treatment. An effective amount can be readily determined by one of skill in the art through the use of known techniques and by observing results obtained under analogous circumstances. In determining the effective amount for an individual, a number of factors are considered including, but not limited to, the species of mammal; its size, age and general health; the specific disease or disorder involved; the degree of or involvement of or the severity of the disease or disorder; the response of the individual patient; the particular polypeptide administered; the mode of administration; the bioavailability characteristics of the preparation administered; the dose regimen selected; the use of concomitant medication; and other relevant circumstances.

As used herein, “extended duration of action” means that binding affinity and activity for a polypeptide continues for a period of time greater than native human GIP, GLP-1 and/or glucagon, allowing for dosing at least as infrequently as once daily, thrice-weekly, twice-weekly, once-weekly, bi-weekly (every two weeks) or once monthly. The time action profile of the polypeptide may be measured using known pharmacokinetic test methods such as those utilized in the examples below.

As used herein, “polypeptide” or “peptide” means a polymer composed of multiple amino acid residues joined covalently in a specific sequence. These amino acids are linked via amide (peptide) bonds, which form when the carboxyl group of one amino acid reacts with the amino group of another, releasing a molecule of water. The term applies to polymers comprising naturally occurring amino acids and polymers comprising one or more non-naturally occurring amino acids.

As used herein, “individual in need thereof” means a mammal, such as a human, with a condition, disease, disorder or symptom requiring treatment or therapy, including for example, those listed herein.

As used herein, “treat,” “treating,” “to treat” and the like mean restraining, slowing, stopping or reversing the progression or severity of an existing condition, disease, disorder or symptom.

As used herein, and with reference to a compound, “dual agonist activity” means a compound with activity at each of the GIP and GLP-1 receptors, especially a polypeptide having sufficient activity at each receptor to provide the benefits of agonism of that receptor while avoiding unwanted side effects associated with too much activity. The polypeptides having dual agonist activity have extended duration of action at the GIP and GLP-1 receptors, which advantageously allows for dosing as infrequently as once-a-week, twice-monthly, once-monthly or once-quarterly.

As used herein, and with reference to a compound, “triple agonist activity” means a compound with activity at each of the GIP, GLP-1 and glucagon receptors, especially a polypeptide having sufficient activity at each receptor to provide the benefits of agonism of that receptor while avoiding unwanted side effects associated with too much activity. The polypeptides having triple agonist activity have extended duration of action at the GIP, GLP-1 and glucagon receptors, which advantageously allows for dosing as infrequently as once-a-week, twice-monthly, once-monthly or once-quarterly.

As used herein, the term “Sequence identity” refers to the degree of similarity between two sequences. The degree of sequence identity between two polypeptides may be expressed as a percent, calculated as follows: % Sequence identity=100%*(number of identical amino acids)/(length of the shortest common sequence).

In certain embodiments of polypeptides of any of the formulas described herein, the polypeptide is an isotopic derivative of any one of the polypeptides described herein or a pharmaceutically acceptable salt thereof. It is understood that the isotopic derivative can be prepared using any of a variety of art-recognized techniques. For example, the isotopic derivatives can generally be prepared by carrying out the procedures disclosed in the examples described herein by substituting an isotopically labeled reagent for a non-isotopically labeled reagent. In an embodiment of a polypeptide of any of the formulas described herein, or a pharmaceutically acceptable salt thereof, the polypeptide is a deuterated derivative of any one of the polypeptides described herein.

In the polypeptides of this invention any atom not specifically designated as a particular isotope is meant to represent any stable isotope of that atom. Unless otherwise stated, when an atom is designated specifically as “H” or “hydrogen”, the atom is understood to have hydrogen at its natural abundance isotopic composition. Also, unless otherwise stated, when an atom is designated specifically as “D” or “deuterium”, the atom is understood to have deuterium at an abundance substantially greater than the natural abundance of deuterium, which is 0.015%.

The polypeptides described herein may react with any number of inorganic and organic acids/bases to form pharmaceutically acceptable acid/base addition salts. Pharmaceutically acceptable salts and common techniques for preparing them are well known in the art (see, e.g., Stahl et al., Handbook of Pharmaceutical Salts: Properties. Selection and Use, 2^{nd} Revised Edition (Wiley-VCH, 2011)). Pharmaceutically acceptable salts for use herein include sodium, potassium, trifluoroacetate, hydrochloride and/or acetate salts. Thus, in some embodiments, provided herein are pharmaceutically acceptable salt forms of polypeptides. In some embodiments, the pharmaceutically acceptable forms are selected from sodium or potassium salts. In some embodiments, the pharmaceutically acceptable forms are selected from the group consisting of sodium, potassium salts. In some embodiments, a pharmaceutically acceptable salt is a sodium salt.

In another embodiment, provided herein is a pharmaceutical composition comprising a polypeptide described herein, or a pharmaceutically acceptable salt thereof, and a pharmaceutically acceptable carrier, diluent, or excipient. Some pharmaceutical compositions and techniques for preparing the same are well known in the art. See, e.g., Remington: The Science and Practice of Pharmacy (Troy, Ed., 21^{st} Edition, Lippincott, Williams & Wilkins, 2006).

In some embodiments, the pharmaceutical composition is suitable for administration by a parenteral route (e.g., subcutaneous, intravenous, intraperitoneal, intramuscular or transdermal). In some embodiments, the pharmaceutical composition is suitable for oral administration (e.g., tablet, capsule). In some embodiments, the pharmaceutical composition is administered parenterally. In some embodiments, the pharmaceutical composition is administered orally.

The disclosure also provides and therefore encompasses novel intermediates and methods of synthesizing the polypeptides described herein, or pharmaceutically acceptable salts thereof. The intermediates and polypeptides described herein can be prepared by a variety of techniques known in the art. For example, a method using chemical synthesis is illustrated in the Examples below or using biological expression. The specific synthetic steps for each of the routes described may be combined in different ways to prepare the polypeptides described herein. The reagents and starting materials are readily available to one of skill in the art.

With respect to chemical synthesis, one can use standard manual or automated solid-phase synthesis procedures. For example, automated peptide synthesizers are commercially available from, for example, CEM (Charlotte, North Carolina), CSBio (Menlo Park, California) and Gyros Protein Technologies Inc. (Tucson, AZ). Reagents for solid-phase synthesis are readily available from commercial sources. Solid-phase synthesizers can be used according to the manufacturer's instructions for blocking interfering groups, protecting amino acids during reaction, coupling, deprotecting and capping of unreacted amino acids.

The compounds disclosed herein include all possible stereoisomers, geometric isomers (e.g., cis/trans), and structural isomers unless specifically stated otherwise. Such isomers may exist due to chiral centers, double bonds, or other stereogenic elements within the molecular structure. Unless otherwise indicated, the nomenclature used and the structures represented are intended to encompass racemic mixtures, individual enantiomers, diastereomers, positional isomers, and regioisomers, as well as mixtures thereof. The invention extends to any pharmaceutically acceptable form of these compounds, including salts, solvates, and polymorphs, regardless of their isomeric composition.

With respect to biological expression, one can use standard recombinant techniques to construct a polynucleotide having a nucleic acid sequence that encodes an amino acid sequence for all or part of a polypeptide, incorporate that polynucleotide into recombinant expression vectors, and introduce the vectors into host cells, such as bacteria, yeast and mammalian cells, to produce the polypeptide. See, e.g., Green & Sambrook, “Molecular Cloning: A Laboratory Manual” (Cold Spring Harbor Laboratory Press, 4th ed. 2012). The polypeptides may readily be produced in mammalian cells such as CHO, NSO, 20 HEK293, BHK, or COS cells; in bacterial cells such as E. coli, Bacillus subtilis, or Pseudomonas fluorescens; in insect cells, or in fungal or yeast cells, which are cultured using techniques known in the art. The vectors containing the polynucleotide sequences of interest can be transferred into the host cell by well-known methods, which vary depending on the type of cellular host. Various methods of protein purification may be employed and such methods are known in the art.

The compounds described herein may be used for treating a variety of conditions, disorders, diseases or symptoms. In particular, methods are provided for treating obesity in an individual, where such methods include at least a step of administering to an individual in need of such treatment an effective amount of a conjugate described herein, or a pharmaceutically acceptable salt thereof.

Additionally, methods are provided for chronic weight management in an individual, where such methods include at least a step of administering to an individual in need of such treatment an effective amount of a conjugate described herein, or a pharmaceutically acceptable salt thereof.

Additionally, methods are provided for treating type 2 diabetes mellitus (T2DM) in an individual, where such methods include at least a step of administering to an individual in need of such treatment an effective amount of a conjugate described herein, or a pharmaceutically acceptable salt thereof.

Additionally, methods are provided for treating non-alcoholic fatty liver disease (NAFLD) in an individual, where such methods include at least a step of administering to an individual in need of such treatment an effective amount of a conjugate described herein, or a pharmaceutically acceptable salt thereof.

Additionally, methods are provided for treating non-alcoholic steatohepatitis (NASH) in an individual, where such methods include at least a step of administering to an individual in need of such treatment an effective amount of a conjugate described herein, or a pharmaceutically acceptable salt thereof.

Additionally, methods are provided for treating dyslipidemia in an individual, where such methods include at least a step of administering to an individual in need of such treatment an effective amount of a polypeptide described herein, or a pharmaceutically acceptable salt thereof.

Additionally, methods are provided for treating metabolic syndrome in an individual, where such methods include at least a step of administering to an individual in need of such treatment an effective amount of a conjugate described herein, or a pharmaceutically acceptable salt thereof.

Additionally, methods are provided for treating osteoarthritis (OA) in an individual, where such methods include at least a step of administering to an individual in need of such treatment an effective amount of a conjugate described herein, or a pharmaceutically acceptable salt thereof.

Additionally, methods are provided for treating obesity-related sleep apnea (OSA) in an individual, where such methods include at least a step of administering to an individual in need of such treatment an effective amount of a conjugate described herein, or a pharmaceutically acceptable salt thereof.

Additionally, methods are provided for treating polycystic ovary syndrome (PCOS) in an individual, where such methods include at least a step of administering to an individual in need of such treatment an effective amount of a conjugate described herein, or a pharmaceutically acceptable salt thereof.

Additionally, methods are provided for inducing non-therapeutic weight loss in an individual, where such methods include at least a step of administering to an individual in need of such treatment an effective amount of a compound described herein, or a pharmaceutically acceptable salt thereof.

In these methods, effectiveness of the compounds can be assessed by, for example, observing a significant reduction in blood glucose, observing a significant increase in insulin, observing a significant reduction in HbA1c and/or observing a significant reduction in body weight.

Alternatively, the compounds described herein or pharmaceutically acceptable salts thereof may be used for improving bone strength in an individual in need thereof. In some instances, the individual in need thereof has hypo-ostosis or hypo-osteoidosis, or is healing from bone fracture, orthotic procedure, prosthetics implant, dental implant, and/or spinal fusion. The polypeptides described herein also may be used for treating other disorders such as Parkinson's disease or Alzheimer's disease.

Additionally, provided are compounds described herein, or a pharmaceutically acceptable salt thereof, for use in therapy. In some embodiments, provided herein is a polypeptide described herein or a pharmaceutically acceptable salt thereof, for use in treating obesity, chronic weight management, type 2 diabetes mellitus, non-alcoholic fatty liver disease (NAFLD), non-alcoholic steatohepatitis (NASH), metabolic dysfunction-associated steatohepatitis (MASH), dyslipidemia, metabolic syndrome, osteoarthritis (OA), obesity-related sleep apnea (OSA) and polycystic ovary syndrome (PCOS). Also provided is a use of a polypeptide described herein, or a pharmaceutically acceptable salt thereof, for inducing non-therapeutic weight loss.

Additionally, provided is a use of the compounds described herein, or a pharmaceutically acceptable salt thereof, in the manufacture of a medicament for treating obesity, chronic weight management, type 2 diabetes mellitus, non-alcoholic fatty liver disease (NAFLD), non-alcoholic steatohepatitis (NASH), dyslipidemia, metabolic syndrome, osteoarthritis (OA), obesity-related sleep apnea (OSA) and polycystic ovary syndrome (PCOS). Also provided is a use of a polypeptide described herein, or a pharmaceutically acceptable salt thereof, in the manufacture of a medicament for inducing non-therapeutic weight loss.

The polypeptides, compounds, or pharmaceutical compositions described herein may be provided as part of a kit. In some instances, the kit includes a device for administering at least one polypeptide (and optionally at least one additional therapeutic agent) to an individual, such as a syringe, automatic injector or pump. Unless defined otherwise, all technical and scientific terms used herein have the same meaning as commonly understood by one of skill in the art to which the disclosure pertains. Although any methods and materials similar to or equivalent to those described herein can be used in the practice or testing of the polypeptides, pharmaceutical compositions, and methods, the preferred methods and materials are described herein.

Reference to an element by the indefinite article “a” or “an” does not exclude the possibility that more than one element is present, unless the context clearly requires that there be one and only one element. The indefinite article “a” or “an” thus usually means “at least one.”

An additional nonlimiting list of embodiments is provided below:

Embodiment 1. A compound of the formula:

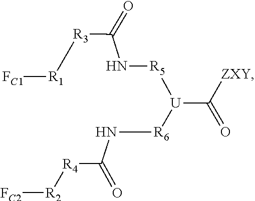

or a pharmaceutically acceptable salt thereof, wherein:
R₁ and R₂ are independently selected from the group consisting of:

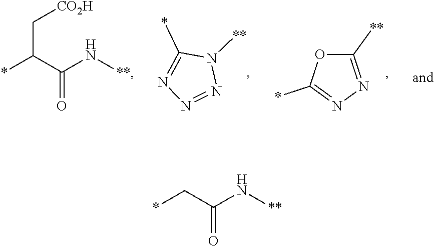

R³ and R⁴ independently comprise a C₁ to C₅ alkyl which is optionally substituted with one or more of NH₂, CH₂NH₂, and CH₂CH₂NH₂,
*is a connection point to a sulfur atom in a cysteine residue in Fc₁ or Fc₂,
**is the connection point to R₅ or R₆,
R₅ and R₆ are independently selected from a covalent bond, or C₁ to C₅ alkyl,
U comprises a tertiary amine or 1,3,5-substituted phenyl,
Z comprises O, NH, C₁ to C₃₀ alkyl, (OCH₂CH₂)ₘ, an amino acid polymer, or a combination thereof,
X comprises a therapeutic polypeptide,
Y comprises a fatty acid,
m is an integer between 1-30; and
Fc₁ and Fc₂ each comprise an Fc region.

Embodiment 2. The compound of embodiment 1, wherein the compound comprises:

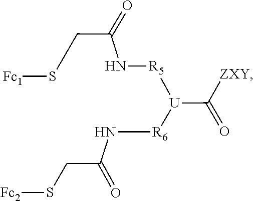

or a pharmaceutically acceptable salt thereof.

Embodiment 3. The compound of embodiment 1, wherein the compound comprises:

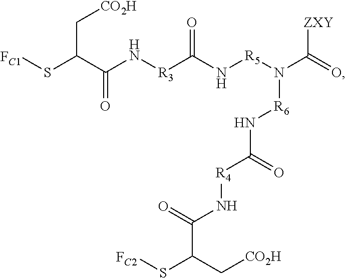

or a pharmaceutically acceptable salt thereof.

Embodiment 4. The compound of embodiment 1, wherein the compound comprises:

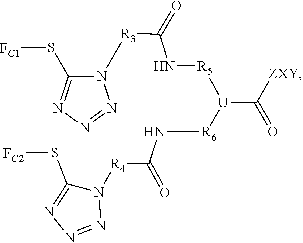

or a pharmaceutically acceptable salt thereof.

Embodiment 5. The compound of embodiment 1, wherein the compound comprises:

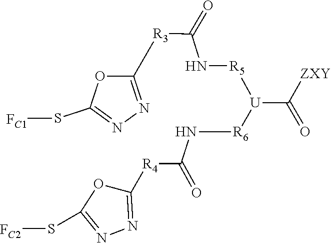

or a pharmaceutically acceptable salt thereof.

Embodiment 6. The compound of any one of embodiments 1-5, wherein the polypeptide comprises:

X₁X₂X₃X₄TX₆TSDX₁₀X₁₁X₁₂X₁₃LX₁₅X₁₆KAQX₂₀X₂₁FIX₂₄X₂₅
LX₂₇X₂₈X₂₉X₃₀X₃₁X₃₂X₃₃X₃₄X₃₅X₃₆X₃₇X₃₈X₃₉X₄₀X₄₁,

wherein:
X₁ is H, NMeY, or Y,
X₂ is Ac4c, Aib, αMeS, Iva, or D-Ala,
X₃ is Q, E, or H,
X₄ is G or D-Ala,
X₆ is αMeF(2F), F, or αMeF,
X₁₀ is 4-Pal, Y, or V,
X₁₁ is Aib, S, or αMeS,
X₁₂ is I or S,
X₁₃ is L or αMeL,
X₁₅ is D or E,
X₁₆ is E or Orn,
X₂₀ is Aib, αMeL, Iva, or αMe4Pal,
X₂₁ is E, Q, D, or Orn,
X₂₄ is K, Q, E, D-Glu, or D-Gln,
X₂₅ is αMeY or Y
X₂₇ is I, L, or V,
X₂₈ K, E or A,
X₂₉ is Aib, G, or Q,
X₃₀ is G or S,
X₃₁ is P, G, E, or Orn,
X₃₂ is absent, S or P,
wherein if X₃₂ is S or P, then X₃₃ is S,
wherein if X₃₃ is S, then is X₃₄ is G or Aib,
wherein if X₃₄ is G or Aib, then X₃₅ is absent or A or Orn,
wherein if X₃₅ is A or Orn, then X₃₆ is absent or P,
wherein if X₃₆ is P, then X₃₇ is absent or P,
wherein if X₃₇ is P, then X₃₈ is absent or P,
wherein if X₃₈ is P, then X₃₉ is absent or S, Orn, or G
wherein if X₃₉ is S, Orn, or G, then X₄₀ is absent, K, or G,
wherein if X₄₀ is K or G, then X₄₁ is absent or S or G,
wherein if X₃₂ is absent, then X₃₃ through X₄₁ are also absent,
wherein if X₃₅ is absent, then X₃₆ through X₄₁ are also absent,
wherein if X₃₆ is absent, then X₃₇ through X₄₁ are also absent,
wherein if X₃₇ is absent, then X₃₈ through X₄₁ are also absent,
wherein if X₃₈ is absent, then X₃₉ through X₄₁ are also absent,
wherein if X₃₉ is absent, then X₄₀ and X₄₁ are also absent,
wherein if X₄₀ is absent, then X₄₁ is absent, and
wherein the C-terminal amino acid is optionally amidated, or a pharmaceutically acceptable salt thereof.

Embodiment 7. The compound of embodiment 6, wherein Z is conjugated to the amino acid at any one of positions 24, 28, 31 or 40 of the polypeptide.

Embodiment 8. The compound of any one of embodiments 1-7, or a pharmaceutically acceptable salt thereof, wherein Z comprises an amino acid polymer.

Embodiment 9. The compound of embodiment 8, or a pharmaceutically acceptable salt thereof, wherein the amino acid polymer comprises any of: (GGGGS)n, (SGGGG)n, (GGGGQ)n, (EAAAK)n, (KAAAE)n, (AEEA)n, G(PA)n, G(PA)n, G(PE)n, G(PK)n, (AP)n, (AP)n, or G(EP)n, wherein n is an integer between 1-10.

Embodiment 10. The compound of any of embodiments 1-9, or a pharmaceutically acceptable salt thereof, wherein the lysine at position 17 is conjugated to a C₁₆-C₂₂ fatty acid via a linker between the amino acid and the C₁₆-C₂₂ fatty acid.

Embodiment 11. The compound of embodiment 10, or pharmaceutically acceptable salt thereof, wherein the C₁₆-C₂₂ fatty acid is conjugated to the lysine at position 17 via a linker.

Embodiment 12. The compound of embodiment 11, or a pharmaceutically acceptable salt thereof, wherein the linker comprises one to four amino acids.

Embodiment 13. The compound of embodiment 12, or a pharmaceutically acceptable salt thereof, wherein the amino acids comprised in the linker are Glu, γGlu or a combination thereof.

Embodiment 14. The compound of any of embodiments 11-13, or a pharmaceutically acceptable salt thereof, wherein the linker comprises one to four (2-[2-(2-amino-ethoxy)-ethoxy]-acetyl) moieties.

Embodiment 15. The compound of embodiment 14, or a pharmaceutically acceptable salt thereof, wherein the linker comprises a structure of (γGlu)ₐ-(2-[2-(2-amino-ethoxy)-ethoxy]-acetyl)_{b}-(γGlu)_{c}-CO—(CH₂)ₚ—CO₂H, wherein ₐ is 0 or 1, _{b} is 0, 1 or 2, _{c} is 1, 2 or 3, and ₚ is an integer between 14 to 20.

Embodiment 16. The compound of embodiment 15, or a pharmaceutically acceptable salt thereof, wherein a is 0, b is 1 and c is 1.

Embodiment 17. The compound of any one of embodiments 1-16, or a pharmaceutically acceptable salt thereof, wherein the polypeptide comprises a sequence identity of more than 90% to any of SEQ ID NO: 1-SEQ ID NO:482.

Embodiment 18. The compound of embodiment 17, or a pharmaceutically acceptable salt thereof, wherein the polypeptide comprises a sequence of any of SEQ ID NO:1-SEQ ID NO:482.

Embodiment 19. The compound of any one of embodiments 1-18, or a pharmaceutically acceptable salt thereof, wherein the Fc1 and Fc2 are selected from the group consisting of:

| (SEQ ID NO: 935) | |
|---|---|
| a. | AGCPAPEAAGGPSVFLFPPKPKDTLMISRTPEVTCVVVDVSQEDPEVQFN |
| | WYVDGVEVHNAKTKPREEQFNSTYRVVSVLTVLHQDWLNGKEYKCKVS |
| | NKGLPSSIEKTISKAKGQPREPQVYTLPPSQEEMTKNQVSLTCLVKGFYPS |
| | DIAVEWESNGQPENNYKTTPPVLDSDGSFFLYSRLTVDKSRWQEGNVFSC |
| | SVMHEALHNHYTQKSLSLSLG, |
| (SEQ ID NO: 936) | |
| b. | AGCPAPEAAGGPSVFLFPPKPKDTLMISRTPEVTCVVVDVSQEDPEVKFN |
| | WYVDGVEVHNAKTKPREEQFNSTYRVVSVLIVLHQDWLNGKEYKCKVS |
| | NKGLPSSIEKTISKAKGQPREPQVYTLPPSREEMTKNQVSLTCLVKGFYPS |
| | DIAVEWESNGQPENNYKTTPPVLDSDGSFFLYSRLTVDKSRWQQGNVFSC |
| | SVMHEALHNHYTQKSLSLSLG, |
| (SEQ ID NO: 941) | |
| c. | IGCPAPEAAGGPSVFLFPPKPKDTLMISRTPEVTCVVVDVSQEDPEVKFNW |
| | YVDGVEVHNAKTKPREEQFNSTYRVVSVLTVLHQDWLNGKEYKCKVSN |
| | KGLPSSIEKTISKAKGQPREPQVYTLPPSREEMTKNQVSLTCLVKGFYPSDI |
| | AVEWESNGQPENNYKTTPPVLDSDGSFFLYSRLTVDKSRWQQGNVFSCSV |
| | MHEALHNHYTQKSLSLSLG, |
| (SEQ ID NO: 941) | |
| d. | IGCPAPEAAGGPSVFLFPPKPKDTLMISRTPEVTCVVVDVSQEDPEVKFNW |
| | YVDGVEVHNAKTKPREEQFNSTYRVVSVLTVLHQDWLNGKEYKCKVSN |
| | KGLPSSIEKTISKAKGQPREPQVYTLPPSREEMTKNQVSLTCLVKGFYPSDI |
| | AVEWESNGQPENNYKTTPPVLDSDGSFFLYSRLTVDKSRWQQGNVFSCSV |
| | MHEALHNHYTQKSLSLSLG, |
| (SEQ ID NO: 943) | |
| e. | AGCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVS |
| | NKALAAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSC |
| | SVMHEALHNHYTQKSLSLSPGK, |
| (SEQ ID NO: 944) | |
| f. | AGCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSN |
| | KALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCSV |
| | MHEALHNHYTQKSLSLSPGK, |
| (SEQ ID NO: 1179) | |
| g. | ESKYGPPCPPCPAPEAAGGPSVFLFPPKPKDTLMISRTPEVTCVVVDVSQE |
| | DPEVKFNWYVDGVEVHNAKTKPREEQFNSTYRVVSVLTVLHQDWLNGK |
| | EYKCKVSNKGLPSSIEKTISKAKGQPREPQVYTLPPSREECTKNQVSLTCL |
| | VKGFYPSDIAVEWESNGQPENNYKTTPPVLDSDGSFFLYSRLTVDKSRWQ |
| | QGNVFSCSVMHEALHNHYTQKSLSLSLG, |
| (SEQ ID NO: 1180) | |
| h. | ESKYGPPCPPCPAPEAAGGPSVFLFPPKPKDTLMISRTPEVTCVVVDVSQE |
| | DPEVKFNWYVDGVEVHNAKTKPREEQFNSTYRVVSVLTVLHQDWLNGK |
| | EYKCKVSNKGLPSSIEKTISKAKGQPREPQVYTLPPSREEMTKNQVSLTCL |
| | VKGFYPSDIAVEWESNGQPENNYKTTPPVCDSDGSFFLYSRLTVDKSRWQ |
| | QGNVFSCSVMHEALHNHYTQKSLSLSLG, |
| (SEQ ID NO: 1181) | |
| i. | ESKYGPPCPPCPAPEAAGGPSVFLFPPKPKDTLMISRTPEVTCVVVDVSQE |
| | DPEVKFNWYVDGVEVHNAKTKPREEQFNSTYRVVSVLTVLHQDWLNGK |
| | EYKCKVSNKGLPSSIEKTISKAKGQPREPQVYTLPPSREEMTKNQVSLTCL |
| | VKGFYPSDIAVEWESNGQPENNYKTTPPVLDSDGSFFLYSRLTVDKCRW |
| | QQGNVFSCSVMHEALHNHYTQKSLSLSLG |
| (SEQ ID NO: 948) | |
| j. | ESKYGPPCPPCPAPEAAGGPSVFLFPPKPKDTLMISRTPEVTCVVVDVSQE |
| | DPEVKFNWYVDGVEVHNAKTKPREEQFNSTYRVVSVLTVLHQDWLNGK |
| | EYKCKVSNKGLPSSIEKTISKAKGQPREPQVYTLPPSREEMTKNQVSLTCL |
| | VKGFYPSDIAVEWESNGQPENNYKTTPPVLDSDGSFLLYSKLTVDKSRWQ |
| | QGNVFSCSVMHEALHNHYTQKSLSLSLG. |

Embodiment 20. The compound of any one of embodiments 1-19, or a pharmaceutically acceptable salt thereof, wherein the pharmaceutically acceptable salt is selected from sodium, potassium, trifluoroacetate, hydrochloride or acetate.

Embodiment 21. A compound comprising any of the following Fc-acylated polypeptides:

| Com- | | Fc |
|---|---|---|
| pound | | Region |
| No. | Compound | (SEQ ID) |
| 1 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK[PEG₂₄-BEA-MalDap₂-Fc]-NH₂ | |
| 2 | Y-Aib-EGT-αMeF(2F)-TSD-4Pal-SI-αMeL-LD-Orn-K(AEEA2-γGlu-C18_OH)AQ-Aib-EFIe- | 935 |
| | αMeY-LIEGSPSSGAPPPSK[PEG₂₄-BEA-MalDap₂-Fc]-NH₂ | |
| 3 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSK[BEA-MalDap₂-Fc]-NH₂ | |
| 4 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSGGGGSK[BEA-MalDap₂-Fc]-NH₂ | |
| 5 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPS-AEEA8-K[BEA-MalDap₂-Fc]-NH₂ | |
| 6 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPS-AEEA4-K[BEA-MalDap₂-Fc]-NH₂ | |
| 7 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPS-PEG₂₄-K[BEA-MalDap₂-Fc]-NH₂ | |
| 8 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(AEEA8-BEA-MalDap₂-Fc)-NH₂ | |
| 9 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(AEEA8-GPAPAPAPAPAPA-BEA-MalDap₂-Fc)-NH₂ | |
| 10 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(AEEA6-GPAPAPAPAPAPA-BEA-MalDap₂-Fc)-NH₂ | |
| 11 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(AEEA4-GPAPAPAPAPAPAPAPA-BEA-MalDap₂-Fc)-NH₂ | |
| 12 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK[GPAPAPAPAPAPAPAPA-BEA-MalDap₂-Fc]-NH₂ | |
| 13 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(AEEA4-GKAAAEKAAAEKAAAE-BEA-MalDap₂-Fc)NH₂ | |
| 14 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(LEAEAAAKEAAAKEAAAKEAAAKALE-BEA-MalDap₂-Fc)-NH₂ | |
| 15 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGSPSSGAPPPS-K[PEG₂₄-BEA-MalDap₂-Fc]-NH₂ | |
| 16 | Y-Aib-EGT-αMeF(2F)-TSD-4Pal-Aib-I-αMeL-LDEK(AEEA2-γGlu-C18_OH)AQ-Aib-EFIE-αMeY- | 935 |
| | LIEGSPSSGAPPPS-K[PEG₂₄-BEA-MalDap₂-Fc]-NH₂ | |
| 17 | Y-Aib-EGT-αMeF(2F)-TSD-4Pal-Aib-S-αMeL-LDEK(AEEA2-γGlu-C18_OH)AQ-Aib-EFIE-αMeY- | 935 |
| | LIEGSPSSGAPPPS-K[PEG₂₄-BEA-MalDap₂-Fc]-NH₂ | |
| 18 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGSPSSGAPPPS-K[PEG₂₄-BEA-MalDap₂-Fc]-NH₂ | |
| 19 | Y-Aib-EGT-αMeF(2F)-TSDV-Aib-I-αMeL-LD-EK(AEEA2-γGlu-C18_OH)AQ-Aib-EFIe-αMeY- | 935 |
| | LIEGSPSSGAPPPS-K[PEG₂₄-BEA-MalDap₂-Fc]-NH₂ | |
| 20 | Y-Aib-EGT-αMeF(2F)-TSDV-Aib-I-αMeL-LD-Orn-K(AEEA2-γGlu-C18_OH)AQ-Aib-EFIe-αMeY- | 935 |
| | LIEGSPSSGAPPPS-K[PEG₂₄-BEA-MalDap₂-Fc]-NH₂ | |
| 21 | Y-Aib-EGT-αMeF(2F)-TSDV-Aib-I-αMeL-LD-Orn-K(AEEA2-γGlu-C18_OH)AQ-Aib-EFIE-αMeY- | 935 |
| | LIEGSPSSGAPPPS-K[PEG₂₄-BEA-MalDap₂-Fc]-NH₂ | |
| 22 | Y-Aib-EGT-αMeF(2F)-TSD-4Pal-SI-αMeL-LDEK(AEEA2-γGlu-C18_OH)AQ-Aib-EFIK[PEG₂₄- | 935 |
| | BEA-MalDap₂-Fc]-αMeY-LIEGSPSSGAPPPSGS-NH₂ | |
| 23 | Y-Aib-EGT-αMeF(2F)-TSD-4Pal-SI-αMeL-LDEK(AEEA2-γGlu-C18_OH)AQ-Aib-EFIe-αMeY- | 935 |
| | LIK[PEG₂₄-BEA-MalDap₂-Fc]GSPSSGAPPPSGS-NH₂ | |
| 24 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGGGGSK[BEA-MalDap₂-Fc]-NH₂ | |
| 25 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGGGGSGGGGSK[BEA-MalDap₂-Fc]-NH₂ | |
| 26 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGPGGGGSGGGGSGGGGSK[BEA-MalDap₂-Fc]-NH₂ | |
| 27 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPGGGGSGGGGGGGGSK[BEA-MalDap₂-Fc]-NH₂ | |
| 28 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGAPAPAPAPAPAPK[BEA-MalDap₂-Fc]-NH₂ | |
| 29 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGAPAPAPAPAPAPK[BEA-MalDap₂-Fc]-NH₂ | |
| 30 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGEAAAKEAAAKEAAAKK[BEA-MalDap₂-Fc]-NH₂ | |
| 31 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGEAAAKEAAAKEAAAKK[BEA-MalDap₂-Fc]-NH₂ | |
| 32 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPSK[PEG₂₄-BEA-MalDap₂-Fc]-NH₂ | |
| 33 | Y-Aib-EGT-αMeF(2F)-TSD-4Pal-SI-αMeL-LDEK(ϵK2-γGlu-C18_OH)AQ-Aib-EFIe-αMeY- | 935 |
| | LIEGSPSSGAPPPSK[PEG₂₄-BEA-MalDap₂-Fc]-NH₂ | |
| 34 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGAPAPAPAPK[BEA-MalDap₂-Fc]-NH₂ | |
| 35 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGAPAPAPK[BEA-MalDap₂-Fc]-NH₂ | |
| 36 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGGGGSGAPAPAPK[BEA-MalDap₂-Fc]-NH₂ | |
| 37 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSEAAAKEAAAKEAAAKEAAAKK[BEA-MalDap₂-Fc]-NH₂ | |
| 38 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGEAAAKEAAAKEAAAKK[BEA-MalDap₂-Fc]-NH₂ | |
| 39 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGGGGSGEAAAKEAAAKEAAAKK[BEA-MalDap₂-Fc]-NH₂ | |
| 40 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 41 | Y-Aib-EGT-αMeF(2F)-TSD-4Pal-Aib-I-αMeL-LDEK(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 42 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 43 | Y-Aib-EGT-αMeF(2F)-TSD-V-Aib-I-αMeL-LDEK(AEEA2-γGlu-C18_OH)AQ-Aib-EFIe-αMeY- | 935 |
| | LIEGGGGGGGSGGGGSGGGGSK[BEA-MalDap₂-Fc]-NH₂ | |
| 44 | Y-Aib-EGT-αMeF(2F)-TSD-V-Aib-I-αMeL-LD-Orn-K(AEEA2-γGlu-C18_OH)AQ-Aib-EFIe-αMeY- | 935 |
| | LIEGGGGGGGSGGGGSGGGGSK[BEA-MalDap₂-Fc]-NH₂ | |
| 45 | Y-Aib-EGT-αMeF(2F)-TSD-V-Aib-I-αMeL-LD-Orn-K(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 46 | Y-Aib-EGT-αMeF(2F)-TSD-4Pal-SI-αMeL-LD-Orn-K(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 47 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGGGGGGGSGGGGSGGGGSK[BEA-MalDap₂-Fc]-NH₂ | |
| 48 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 49 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGAPAPAPAPAPAPAPAPK[BEA-MalDap₂-Fc]-NH₂ | |
| 50 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGAPAPAPAPAPAPK[BEA-MalDap₂-Fc]-NH₂ | |
| 51 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGGGGGSGGGGSGGGGSGGGGSGAPAPAPAPAPAPK[BEA-MalDap₂-Fc]-NH₂ | |
| 52 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGQGGGGQGGGGQK[BEA-MalDap₂-Fc]-NH₂ | |
| 53 | Y-Aib-EGT-αMeF(2F)-TSD-4Pal-SI-αMeL-LDEK(AEEA2-γGlu-C18-OH)AQ-Aib-EFIE-αMeY- | 935 |
| | LIEGGGGGGGQGGGGQGGGGQGGGGQK[BEA-MalDap₂-Fc]-NH₂ | |
| 54 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGQGGGGQGGGGQGGGGQGGGGQK[BEA-MalDap₂-Fc]-NH₂ | |
| 55 | Y-Aib-EGT-αMeF(2F)-TSD-4Pal-SI-αMeL-LDEK(AEEA2-γGlu-C18_OH)AQ-Aib-EFIK(AEEA8- | 935 |
| | BEA-MalDap₂-Fc)-αMeY-LIEGSPSSGAPPPSGS-NH₂ | |
| 56 | Y-Aib-EGT-αMeF(2F)-TSD-4Pal-SI-αMeL-LDEK(AEEA2-γGlu-C18_OH)AQ-Aib-EFIE-αMeY- | 935 |
| | LIK(AEEA8-BEA-MalDap₂-Fc)GSPSSGAPPPSGS-NH₂ | |
| 57 | Y-Aib-EGT-αMeF(2F)-TSD-4Pal-SI-αMeL-LDEK(AEEA2-γGlu-C18_OH)AQ-Aib-EFIE-αMeY- | 935 |
| | LIK(GGGGSGGGGSGGGGSGGGGSG-BEA-MalDap₂-Fc)GSPSSGAPPPSGS-NH₂ | |
| 58 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGSGAPAPAPAPAPAPK[BEA-MalDap₂-Fc]-NH₂ | |
| 59 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGSGGGGSGGGGSGAPAPAPAPAPAPK[BEA-MalDap₂-Fc]-NH₂ | |
| 60 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGSGEAAAKEAAAKEAAAKK[BEA-MalDap₂-Fc]-NH₂ | |
| 61 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGSGGGGSGGGGSGEAAAKEAAAKEAAAKK[BEA-MalDap₂-Fc]-NH₂ | |
| 62 | Y-Aib-EGT-αMeF(2F)-TSD-4Pal-Aib-I-αMeL-LDEK(AEEA2-γGlu-C18_OH)AQ-Aib-EFIE-αMeY- | 935 |
| | LIEGGGGGGGSGAPAPAPAPAPAPK[BEA-MalDap₂-Fc]-NH₂ | |
| 63 | Y-Aib-EGT-αMeF(2F)-TSD-4Pal-Aib-I-αMeL-LDEK(AEEA2-γGlu-C18_OH)AQ-Aib-EFIE-αMeY- | 935 |
| | LIEGGGGGGGSGGGGSGGGGSGAPAPAPAPAPAPK[BEA-MalDap₂-Fc]-NH₂ | |
| 64 | Y-Aib-EGT-αMeF(2F)-TSD-4Pal-Aib-I-αMeL-LDEK(AEEA2-γGlu-C18_OH)AQ-Aib-EFIE-αMeY- | 935 |
| | LIEGGGGGGGSGEAAAKEAAAKEAAAKK[BEA-MalDap₂-Fc]-NH₂ | |
| 65 | Y-Aib-EGT-αMeF(2F)-TSD-4Pal-Aib-I-αMeL-LDEK(AEEA2-γGlu-C18_OH)AQ-Aib-EFIE-αMeY- | 935 |
| | LIEGGGGGGGSGGGGSGGGGSGEAAAKEAAAKEAAAKK[BEA-MalDap₂-Fc]-NH₂ | |
| 66 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGSGAPAPAPAPAPAPK[BEA-MalDap₂-Fc]-NH₂ | |
| 67 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGSGGGGSGGGGSGAPAPAPAPAPAPK[BEA-MalDap₂-Fc]-NH₂ | |
| 68 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGSGEAAAKEAAAKEAAAKK[BEA-MalDap₂-Fc]-NH₂ | |
| 69 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGSGGGGSGGGGSGEAAAKEAAAKEAAAKK[BEA-MalDap₂-Fc]-NH₂ | |
| 70 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGSGAPAPAPAPAPAPK[BEA-MalDap₂-Fc]-NH₂ | |
| 71 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGSGGGGSGGGGSGAPAPAPAPAPAPK[BEA-MalDap₂-Fc]-NH₂ | |
| 72 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGSGEAAAKEAAAKEAAAKK[BEA-MalDap₂-Fc]-NH₂ | |
| 73 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGGGAPAPAPAPAPAPGGGGSGGGGSK[BEA-MalDap₂-Fc]-NH₂ | |
| 74 | Y-Aib-EGT-αMeF(2F)-TSD-4Pal-SI-αMeL-LDEK(AEEA2-γGlu-C18_OH)AQ-Aib- | 935 |
| | EFIK(SGGGGSGGGGSGGGGGPAPAPAPAPAPAG-BEA-MalDap₂-Fc)-αMeY-LIEGSPSSGAPPPSGS- | |
| | NH₂ | |
| 75 | Y-Aib-EGT-αMeF(2F)-TSD-4Pal-SI-αMeL-LDEK(AEEA2-γGlu-C18_OH)AQ-Aib- | 935 |
| | EFIK(SGGGGSGGGGSGGGGGKAAAEKAAAEKAAAEG-BEA-MalDap₂-Fc)-αMeY- | |
| | LIEGSPSSGAPPPSGS-NH₂ | |
| 76 | Y-Aib-EGT-αMeF(2F)-TSD-4Pal-SI-αMeL-LDEK(AEEA2-γGlu-C18_OH)AQ-Aib-EFIE-αMeY- | 935 |
| | LIK(SGGGGSGGGGSGGGGGPAPAPAPAPAPAG-BEA-MalDap₂-Fc)GSPSSGAPPPSGS-NH₂ | |
| 77 | Y-Aib-EGT-αMeF(2F)-TSD-4Pal-SI-αMeL-LDEK(AEEA2-γGlu-C18_OH)AQ-Aib-EFIE-αMeY- | 935 |
| | LIK(SGGGGSGGGGSGGGGGKAAAEKAAAEKAAAEG-BEA-MalDap₂-Fc)GSPSSGAPPPSGS-NH₂ | |
| 78 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(AEEA4-GPKPKPKPKPKPK-BEA-MalDap₂-Fc)-NH₂ | |
| 79 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK(AEEA4-GPEPEPEPEPEPE-BEA-MalDap₂-Fc)-NH₂ | |
| 80 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGSGEPEPEPEPEPEPK[BEA-MalDap₂-Fc]-NH₂ | |
| 81 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGSGGGGSGGGGSGEPEPEPEPEPEPK[BEA-MalDap₂-Fc]-NH₂ | |
| 82 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGGGGGGGQGGGGQGGGGQGEPEPEPEPEPEPK[BEA-MalDap₂-Fc]-NH₂ | |
| 83 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGSGEPEPEPEPEPEPK[BEA-MalDap₂-Fc]-NH₂ | |
| 84 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGSGGGGSGGGGSGEPEPEPEPEPEPK(BEA-MalDap₂-Fc)-NH₂ | |
| 85 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGQGGGGQGGGGQGEPEPEPEPEPEPK[BEA-MalDap₂-Fc]-NH₂ | |
| 86 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGSPSSGAPPPS-AEEA2-GEPEPEPEPEPEPK[BEA-MalDap₂-Fc]-NH₂ | |
| 87 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGSPSSGAPPPS-AEEA4-GEPEPEPEPEPEPK[BEA-MalDap₂-Fc]-NH₂ | |
| 88 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGSPSSGAPPPS-AEEA2-GEPEPEPEPEPEPK[BEA-MalDap₂-Fc]-NH₂ | |
| 89 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGSPSSGAPPPS-AEEA4-GEPEPEPEPEPEPK[BEA-MalDap₂-Fc]-NH₂ | |
| 90 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGSPSSGAPPPSK(AEEA6-GPAPAPAPAPAPA-BEA-MalDap₂-Fc)-NH₂ | |
| 91 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGSPSSGAPPPSK(AEEA4-GPEPEPEPEPEPE-BEA(MalDap₂-Fc)-NH₂ | |
| 92 | Y-Aib-EGT-αMeF(2F)-TSD-4Pal-SI-αMeL-LEEK(AEEA2-γGlu-C18_OH)AQ-Aib-EFIE-αMeY- | 935 |
| | LIEGSPSSGAPPPSK(AEEA4-GPEPEPEPEPEPEPEPE-BEA(MalDap₂-Fc)-NH₂ | |
| 93 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGSPSSGAPPPSK(AEEA6-GPAPAPAPAPAPA-BEA(MalDap₂-Fc)-NH₂ | |
| 94 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGSPSSGAPPPSK(AEEA4-GPEPEPEPEPEPE-BEA(MalDap₂-Fc)-NH₂ | |
| 95 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGSPSSGAPPPSK(AEEA4-GPEPEPEPEPEPEPEPE-BEA(MalDap₂-Fc)-NH₂ | |
| 96 | Y-Aib-EGT-αMeF(2F)-TSD-4Pal-SI-αMeL-LD-Orn-K(AEEA2-γGlu-C18_OH)AQ-Aib-EFIE-αMeY- | 935 |
| | LIEGGGGGGGGGGGGSGAPAPAPAPAPAPK[BEA-MalDap₂-Fc]-NH₂ | |
| 97 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGGGGGGGSGGGGSGAPAPAPAPAPAPK[BEA-MalDap₂-Fc]-NH₂ | |
| 98 | Y-Aib-EGT-αMeF(2F)-TSD-4Pal-SI-αMeL-LE-Orn-K(AEEA2-γGlu-C18_OH)AQ-Aib-EFIE-αMeY- | 935 |
| | LIEGGGGGGGSGGGGSGAPAPAPAPAPAPK[BEA-MalDap₂-Fc]-NH₂ | |
| 99 | Y-Aib-EGT-αMeF(2F)-TSD-4Pal-Aib-I-αMeL-LE-Orn-K(AEEA2-γGlu-C18_OH)AQ-Aib-EFIE- | 935 |
| | αMeY-LIEGGGGGGGSGGGGSGAPAPAPAPAPAPK[BEA-MalDap₂-Fc]-NH₂ | |
| 100 | Y-Aib-EGT-αMeF(2F)-TSD-4Pal-Aib-I-αMeL-LD-Orn-K(AEEA2-γGlu-C18_OH)AQ-Aib-EFIe- | 935 |
| | αMeY-LIEGGGGGGGSGGGGSGAPAPAPAPAPAPK[BEA-MalDap₂-Fc]-NH₂ | |
| 101 | Y-Aib-EGT-αMeF(2F)-TSD-4Pal-SI-αMeL-LD-Orn-K(AEEA2-γGlu-C18_OH)AQ-Aib- | 935 |
| | EFIK(GGGGSGGGGSGAPAPAPAPAPAPG-BEA-MalDap₂-Fc)-αMeY-LIEGSPSSGAPPPSGS-NH₂ | |
| 102 | Y-Aib-EGT-αMeF(2F)-TSD-4Pal-Aib-I-αMeL-LD-Orn-K(AEEA2-γGlu-C18_OH)AQ-Aib- | 935 |
| | EFIK(GGGGSGGGGSGAPAPAPAPAPAPG-BEA-MalDap₂-Fc)-αMeY-LIEGSPSSGAPPPSGS-NH₂ | |
| 103 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGPSSGAPPPSGK[PEG₂₄-BEA-MalDap₂-Fc]-NH₂ | |
| 104 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGESSGAPPPSGK[PEG₂₄-BEA-MalDap₂-Fc]-NH₂ | |
| 105 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGG-Orn-SSGAPPPSGK[PEG₂₄-BEA-MalDap₂-Fc]-NH₂ | |
| 106 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGPPSGAPPPSGK[PEG₂₄-BEA-MalDap₂-Fc]-NH₂ | |
| 107 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGPPS-Aib-Orn-PPP-Orn-GK[PEG₂₄-BEA-MalDap₂-Fc]-NH₂ | |
| 108 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGSPSSGAPPPGGK[PEG₂₄-BEA-MalDap₂-Fc]-NH₂ | |
| 109 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGGPSSGAPPPSGG-NH₂ | |
| 110 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGGESSGAPPPSGG-NH₂ | |
| 111 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGG-Orn-SSGAPPPSGG-NH₂ | |
| 112 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGGPPSGAPPPSGG-NH₂ | |
| 113 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGGPPS-Aib-Orn-PPP-Orn-GG-NH₂ | |
| 114 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGSPSSGAPPPGGG-NH₂ | |
| 115 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGGGGGGSGGGGSGGGGSK[BEA-MalDap₂-Fc]-NH₂ | |
| 116 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGPSSGK[BEA-MalDap₂-Fc]-NH₂ | |
| 117 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGG-PEG₂₄-K[BEA-MalDap₂-Fc]-NH₂ | |
| 118 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGGK[PEG₂₄-BEA-MalDap₂-Fc]-NH₂ | |
| 119 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGGG-NH₂ | |
| 120 | Y-Aib-EGT-αMeF(2F)-TSD-4Pal-Aib-I-αMeL-LD-Orn-K(ϵK2-γGlu-C18_OH)AQ-Aib-EFIK | 935 |
| | [PEG₂₄-BEA-MalDap₂-Fc]-αMeY-LIEGGPSSG-NH₂ | |
| 121 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIK[PEG₂₄-BEA-MalDap₂-Fc]GGG-NH₂ | |
| 122 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIK[PEG₂₄-BEA-MalDap₂-Fc]GGPSSG-NH₂ | |
| 123 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGGGGGGSGGGGSGAPAPAPAPAPAPK[BEA-MalDap₂-Fc]-NH₂ | |
| 124 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGGGGGGQGGGGQGGGGQK[BEA-MalDap₂-Fc]-NH₂ | |
| 125 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGGGGGG-4Pal-GGGG-4Pal-GGGG-4Pal-K[BEA-MalDap₂-Fc]-NH₂ | |
| 126 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGPSSGK[PEG₂₄-BEA-MalDap₂-Fc]-NH₂ | |
| 127 | Y-Aib-EGT-αMeF(2F)-TSD-4Pal-Aib-I-αMeL-LD-Orn-K(AEEA2-γGlu-C18_OH)AQ-Aib-EFIK | 935 |
| | [PEG₂₄-BEA-MalDap₂-Fc]-αMeY-LIEGGG-NH₂ | |
| 128 | Y-Aib-EGT-αMeF(2F)-TSD-4Pal-Aib-I-αMeL-LD-Orn-K(AEEA2-γGlu-C18_OH)AQ-Aib-EFIK | 935 |
| | [PEG₂₄-BEA-MalDap₂-Fc]-αMeY-LIEGGPSSG-NH₂ | |
| 129 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIK[PEG₂₄-BEA-MalDap₂-Fc]GGG-NH₂ | |
| 130 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIK[PEG₂₄-BEA-MalDap₂-Fc]GGPSSG-NH₂ | |
| 131 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGGPSSGK[PEG₂₄-BEA-MalDap₂-Fc]-NH₂ | |
| 132 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGGG-PEG₂₄-K[BEA-MalDap₂-Fc]-NH₂ | |
| 133 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK[PEG₂₄-BEA-MalDap₂-Fc]-NH₂ | |
| 134 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGSPSSGAPPPSK[PEG₂₄-BEA-MalDap₂-Fc]-NH₂ | |
| 135 | Y-Aib-EGT-αMeF(2F)-TSD-4Pal-Aib-I-αMeL-LDEK(ϵK2-γGlu-C18_OH)AQ-Aib-EFIE-αMeY- | 935 |
| | LIEGSPSSGAPPPSK[PEG₂₄-BEA-MalDap₂-Fc]-NH₂ | |
| 136 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGGPSSGAPPPSK[PEG₂₄-BEA-MalDap₂-Fc]-NH₂ | |
| 137 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGGPSSGAPPPSK[PEG₂₄-BEA-MalDap₂-Fc]-NH₂ | |
| 138 | Y-Aib-EGT-αMeF(2F)-TSD-4Pal-Aib-I-αMeL-LDEK(ϵK2-γGlu-C18_OH)AQ-Aib-EFIE-αMeY- | 935 |
| | LIEGGPSSGAPPPSK[PEG₂₄-BEA-MalDap₂-Fc]-NH₂ | |
| 139 | Y-Aib-EGT-αMeF(2F)-TSD-4Pal-Aib-I-αMeL-LD-Orn-K(ϵK2-γGlu-C18_OH)AQ-Aib-EFIe- | 935 |
| | αMeY-LIEGSPSSGAPPPSK(PEG₂₄-BEA-MalDap₂ Fc)-NH₂ | |
| 140 | Y-Aib-EGT-αMeF(2F)-TSD-4Pal-Aib-I-αMeL-LD-Orn-K(ϵK2-γGlu-C18_OH)AQ-Aib-EFIe- | 935 |
| | αMeY-LIEGGGK(PEG₂₄-BEA-MalDap₂ Fc)-NH₂ | |
| 141 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGG-Orn-SSGAPPPSK(PEG₂₄-BEA-MalDap₂ Fc)-NH₂ | |
| 142 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGG-Orn-SSGAPPPSK(PEG₂₄-BEA-MalDap₂ Fc)-NH₂ | |
| 143 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGG-Orn-K(PEG₂₄-BEA-MalDap₂ Fc)-NH₂ | |
| 144 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGG-Orn-K(PEG₂₄-BEA-MalDap₂ Fc)-NH₂ | |
| 145 | Y-Aib-EGT-αMeF(2F)-TSD-4Pal-Aib-I-αMeL-LD-Orn-K(ϵK2-γGlu-C18_OH)AQ-Aib-Orn-FIE- | 935 |
| | αMeY-LIEGSPSSGAPPPSK(PEG₂₄-BEA-MalDap₂ Fc)-NH₂ | |
| 146 | Y-Aib-EGT-αMeF(2F)-TSD-4Pal-Aib-I-αMeL-LD-Orn-K(AEEA2-γGlu-C18_OH)AQ-Aib-Orn- | 933 |
| | FIE-αMeY-LIEGSPSSGAPPPSK(PEG₂₄-BEA-MalDap₂ Fc)-NH₂ | |
| 147 | Y-Aib-EGT-αMeF(2F)-TSD-4Pal-Aib-I-αMeL-LD-Orn-K(AEEA2-γGlu-C18_OH)AQ-Aib-EFIQ- | 935 |
| | αMeY-LIEGSPSSGAPPPSK(PEG₂₄-BEA-MalDap₂ Fc)-NH₂ | |
| 148 | Y-Aib-EGT-αMeF(2F)-TSD-4Pal-Aib-I-αMeL-LD-Orn-K(ϵK2-γGlu-C18_OH)AQ-Aib-EFIQ- | 935 |
| | αMeY-LIEGSPSSGAPPPSK(PEG₂₄-BEA-MalDap₂ Fc)-NH₂ | |
| 149 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGSPSSGAPPPSK(AEEA6-GPAPAPAPAPAPA-BEA-MalDap₂-Fc)-NH₂ | |
| 150 | Y-Aib-EGT-αMeF(2F)-TSD-4Pal-Aib-I-αMeL-LD-Orn-K(ϵK2-γGlu-C18_OH)AQ-Aib-EFIe- | 935 |
| | αMeY-LIEGSPSSGAPPPSK(AEEA6-GPAPAPAPAPAPA-BEA-MalDap₂-Fc)-NH₂ | |
| 151 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA4-GPAPAPAPAPAPA-BEA-MalDap₂-Fc)-NH₂ | |
| 152 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA4-GPAPAPAPA-BEA-MalDap₂-Fc)-NH₂ | |
| 153 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA4-GPAPA-BEA-MalDap₂-Fc)-NH₂ | |
| 154 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA6-GPAPAPAPAPAPA-BEA-MalDap₂-Fc)-NH₂ | |
| 155 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA6-GPAPAPAPA-BEA-MalDap₂-Fc)-NH₂ | |
| 156 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA6-GPAPA-BEA-MalDap₂-Fc)-NH₂ | |
| 157 | Y-Aib-EGT-αMeF(2F)-TSD-4Pal-SI-αMeL-LD-Orn-K(AEEA2-γGlu-C18_OH)AQ-Aib-EFIE-αMeY- | 935 |
| | LIEGSPSSGAPPPSK(AEEA6-GPAPAPAPAPAPA-BEA-MalDap₂-Fc)-NH₂ | |
| 158 | Y-Aib-EGT-αMeF(2F)-TSD-4Pal-SI-αMeL-LD-Orn-K(ϵK2-γGlu-C18_OH)AQ-Aib-EFIE-αMeY- | 935 |
| | LIEGSPSSGAPPPSK(AEEA6-GPAPAPAPAPAPA-BEA-MalDap₂-Fc)-NH₂ | |
| 159 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPSK(AEEA6-GPAPAPAPAPAPA-BEA-MalDap₂-Fc)-NH₂ | |
| 160 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA4-BEA-MalDap₂-Fc)-NH₂ | |
| 161 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA6-BEA-MalDap₂-Fc)-NH₂ | |
| 162 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGSPSSGAPPPSK(AEEA8-BEA-MalDap₂-Fc)-NH₂ | |
| 163 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIE-Aib-SPSSGAPPPSK[PEG₂₄-BEA-MalDap₂-Fc]-NH₂ | |
| 164 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIE-Aib-SPSSGAPPPSK[PEG₂₄-BEA-MalDap₂-Fc]-NH₂ | |
| 165 | Y-Aib-EGT-αMeF(2F)-TSD-4Pal-Aib-I-αMeL-LD-Orn-K(ϵK2-γGlu-C18_OH)AQ-Aib-EFIe- | 935 |
| | αMeY-LIE-Aib-SPSSGAPPPSK[PEG₂₄-BEA-MalDap₂-Fc]-NH₂ | |
| 166 | Y-Aib-EGT-αMeF(2F)-TSD-4Pal-SI-αMeL-LD-Orn-K(AEEA2-γGlu-C18_OH)AQ-Aib-EFIE-αMeY- | 935 |
| | LIEGSPSSGAPPPSK[PEG₂₄-BEA-MalDap₂-Fc]-NH₂ | |
| 167 | Y-Aib-EGT-αMeF(2F)-TSD-4Pal-SI-αMeL-LD-Orn-K(AEEA2-γGlu-C18_OH)AQ-Aib-EFIQ-αMeY- | 935 |
| | LIEGSPSSGAPPPSK[PEG₂₄-BEA-MalDap₂-Fc]-NH₂ | |
| 168 | Y-Aib-EGT-αMeF(2F)-TSD-4Pal-SI-αMeL-LD-Orn-K(ϵK2-γGlu-C18_OH)AQ-Aib-EFIE-αMeY- | 935 |
| | LIEGSPSSGAPPPSK[PEG₂₄-BEA-MalDap₂-Fc]-NH₂ | |
| 169 | Y-Aib-EGT-αMeF(2F)-TSD-4Pal-SI-αMeL-LD-Orn-K(ϵK2-γGlu-C18_OH)AQ-Aib-EFIQ-αMeY- | 935 |
| | LIEGSPSSGAPPPSK[PEG₂₄-BEA-MalDap₂-Fc]-NH₂ | |
| 170 | Y-Aib-EGT-αMeF(2F)-TSDY-Aib-I-αMeL-LD-Orn-K(AEEA2-γGlu-C18_OH)AQ-Aib-EFIE-αMeY- | 935 |
| | LIEGSPSSGAPPPSK[PEG₂₄-BEA-MalDap₂-Fc]-NH₂ | |
| 171 | Y-Aib-EGT-αMeF(2F)-TSDY-Aib-I-αMeL-LD-Orn-K(AEEA2-γGlu-C18_OH)AQ-Aib-EFIQ-αMeY- | 935 |
| | LIEGSPSSGAPPPSK[PEG₂₄-BEA-MalDap₂-Fc]-NH₂ | |
| 172 | Y-Aib-EGT-αMeF(2F)-TSDY-Aib-I-αMeL-LD-Orn-K(ϵK2-γGlu-C18_OH)AQ-Aib-EFIe-αMeY- | 935 |
| | LIEGSPSSGAPPPSK[PEG₂₄-BEA-MalDap₂-Fc]-NH₂ | |
| 173 | Y-Aib-EGT-αMeF(2F)-TSDY-Aib-I-αMeL-LD-Orn-K(ϵK2-γGlu-C18_OH)AQ-Aib-EFIE-αMeY- | 935 |
| | LIEGSPSSGAPPPSK[PEG₂₄-BEA-MalDap₂-Fc]-NH₂ | |
| 174 | Y-Aib-EGT-αMeF(2F)-TSDY-Aib-I-αMeL-LD-Orn-K(ϵK2-γGlu-C18_OH)AQ-Aib-EFIQ-αMeY- | 935 |
| | LIEGSPSSGAPPPSK[PEG₂₄-BEA-MalDap₂-Fc]-NH₂ | |
| 175 | Y-Aib-EGT-αMeF(2F)-TSD-4Pal-Aib-I-αMeL-LD-Orn-K(ϵK2-γGlu-C18_OH)AQ-αMeL-EFIE- | 935 |
| | αMeY-LIEGSPSSGAPPPSK[PEG₂₄-BEA-MalDap₂-Fc]-NH₂ | |
| 176 | Y-Aib-EGT-αMeF(2F)-TSD-4Pal-Aib-I-αMeL-LD-Orn-K(ϵK2-γGlu-C18_OH)AQ-Iva-EFIE- | 935 |
| | αMeY-LIEGSPSSGAPPPSK[PEG₂₄-BEA-MalDap₂-Fc]-NH₂ | |
| 177 | Y-Aib-EGT-αMeF(2F)-TSD-4Pal-Aib-I-αMeL-LD-Orn-K(AEEA2-γGlu-C18_OH)AQ-αMeL-EFIQ- | 935 |
| | αMeY-LIEGSPSSGAPPPSK[PEG₂₄-BEA-MalDap₂-Fc]-NH₂ | |
| 178 | Y-Aib-EGT-αMeF(2F)-TSD-4Pal-Aib-I-αMeL-LD-Orn-K(AEEA2-γGlu-C18_OH)AQ-Iva-EFIQ- | 935 |
| | αMeY-LIEGSPSSGAPPPSK[PEG₂₄-BEA-MalDap₂-Fc]-NH₂ | |
| 179 | Y-Aib-EGT-αMeF(2F)-TSD-4Pal-Aib-I-αMeL-LD-Orn-K(ϵK2-γGlu-C18_OH)AQ-αMeL-EFIQ- | 935 |
| | αMeY-LIEGSPSSGAPPPSK[PEG₂₄-BEA-MalDap₂-Fc]-NH₂ | |
| 180 | Y-Aib-EGT-αMeF(2F)-TSD-4Pal-Aib-I-αMeL-LD-Orn-K(ϵK2-γGlu-C18_OH)AQ-Iva-EFIQ- | 935 |
| | αMeY-LIEGSPSSGAPPPSK[PEG₂₄-BEA-MalDap₂-Fc]-NH₂ | |
| 181 | Y-Aib-EGT-αMeF(2F)-TSD-4Pal-Aib-I-αMeL-LD-Orn-K(AEEA2-γGlu-C18_OH)AQ-Aib-QFIE- | 935 |
| | αMeY-LIEGSPSSGAPPPSK[PEG₂₄-BEA-MalDap₂-Fc]-NH₂ | |
| 182 | Y-Aib-EGT-αMeF(2F)-TSD-4Pal-Aib-I-αMeL-LD-Orn-K(ϵK2-γGlu-C18_OH)AQ-Aib-EFIQ- | 935 |
| | αMeY-LLEGSPSSGAPPPSK[PEG₂₄-BEA-MalDap₂-Fc]-NH₂ | |
| 183 | Y-Aib-EGT-αMeF(2F)-TSD-4Pal-SI-αMeL-LD-Orn-K(AEEA2-γGlu-C18_OH)AQ-Aib-EFIQ-αMeY- | 935 |
| | LIEGSPSSGAPPPSK(AEEA6-GPAPAPAPAPAPA-BEA-MalDap₂-Fc)-NH₂ | |
| 184 | Y-Aib-EGT-αMeF(2F)-TSD-4Pal-SI-αMeL-LD-Orn-K(AEEA2-γGlu-C18_OH)AQ-Aib-EFIQ-αMeY- | 935 |
| | LIEGSPSSGAPPPSK(2-[2-(2-amino-ethoxy)-ethoxy]-acetyl)-BEA-MalDap₂-Fc)-NH₂ | |
| 185 | Y-Aib-EGT-αMeF(2F)-TSD-4Pal-SI-αMeL-LD-Orn-K(ϵK2-γGlu-C18_OH)AQ-Aib-EFIQ-αMeY- | 935 |
| | LIEGSPSSGAPPPSK(2-[2-(2-amino-ethoxy)-ethoxy]-acetyl)₆-BEA-MalDap₂-Fc)-NH₂ | |
| 186 | Y-Aib-EGT-αMeF(2F)-TSD-4Pal-SI-αMeL-LD-Orn-K(AEEA2-γGlu-C18_OH)AQ-Aib-EFIE-αMeY- | 935 |
| | LIEGGGGGGGSGGGGSGAPAPAPAPAPAPK[BEA-MalDap₂-Fc]-NH₂ | |
| 187 | Y-Aib-EGT-αMeF(2F)-TSD-4Pal-SI-αMeL-LD-Orn-K(AEEA2-γGlu-C18_OH)AQ-Aib-EFIQ-αMeY- | 935 |
| | LIEGGGGGGGSGGGGSGGGGSK[BEA-MalDap₂-Fc]-NH₂ | |
| 188 | Y-Aib-EGT-αMeF(2F)-TSD-4Pal-SI-αMeL-LD-Orn-K(AEEA2-γGlu-C18_OH)AQ-Aib-EFIQ-αMeY- | 935 |
| | LIEGGGGGGGSGGGGSGAPAPAPAPAPAPK[BEA-MalDap₂-Fc]-NH₂ | |
| 189 | Y-Aib-EGT-αMeF(2F)-TSD-4Pal-SI-αMeL-LD-Orn-K(ϵK2-γGlu-C18_OH)AQ-Aib-EFIE-αMeY- | 935 |
| | LIEGGGGGGGSGGGGGGGGSK[BEA-MalDap₂-Fc]-NH₂ | |
| 190 | Y-Aib-EGT-αMeF(2F)-TSD-4Pal-SI-αMeL-LD-Orn-K(ϵK2-γGlu-C18_OH)AQ-Aib-EFIQ-αMeY- | 935 |
| | LIEGGGGGGGSGGGGSGGGGSK[BEA-MalDap₂-Fc]-NH₂ | |
| 191 | Y-Aib-EGT-αMeF(2F)-TSD-4Pal-SI-αMeL-LD-Orn-K(ϵK2-γGlu-C18_OH)AQ-Aib-EFIQ-αMeY- | 935 |
| | LIEGGGGGGGSGGGGSGAPAPAPAPAPAPK[BEA-MalDap₂-Fc]-NH₂ | |
| 192 | Y-Aib-EGT-αMeF(2F)-TSD-4Pal-Aib-I-αMeL-LD-Orn-K(AEEA2-γGlu-C18_OH)AQ-Aib-EFIQ- | 935 |
| | αMeY-LIEGSPSSGAPPPSK(AEEA6-GPAPAPAPAPAPA-BEA-MalDap₂-Fc)-NH₂ | |
| 193 | Y-Aib-EGT-αMeF(2F)-TSD-4Pal-Aib-I-αMeL-LD-Orn-K(AEEA2-γGlu-C18_OH)AQ-Aib-EFIQ- | 935 |
| | αMeY-LIEGSPSSGAPPPSK(2-[2-(2-amino-ethoxy)-ethoxy]-acetyl)-BEA-MalDap₂-Fc)-NH₂ | |
| 194 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEGGGGGGGSGGGGSGAPAPAPAPAPAPK[BEA-MalDap₂-Fc]-NH₂ | |
| 195 | Y-Aib-EGT-αMeF(2F)-TSD-4Pal-Aib-I-αMeL-LD-Orn-K(AEEA2-γGlu-C18_OH)AQ-Aib-EFIQ- | 935 |
| | αMeY-LIEGGGGGGGSGGGGSGGGGSK[BEA-MalDap₂-Fc]-NH₂ | |
| 196 | Y-Aib-EGT-αMeF(2F)-TSD-4Pal-Aib-I-αMeL-LD-Orn-K(AEEA2-γGlu-C18_OH)AQ-Aib-EFIQ- | 935 |
| | αMeY-LIEGGGGGGGSGGGGSGAPAPAPAPAPAPK[BEA-MalDap₂-Fc]-NH₂ | |
| 197 | Y-Aib-EGT-αMeF(2F)-TSD-4Pal-Aib-I-αMeL-LD-Orn-K(AEEA2-γGlu-C18_OH)AQ-Aib-DFIQ- | 935 |
| | αMeY-LIEGSPSSGAPPPSK(AEEA6-BEA-MalDap₂)-NH₂ | |
| 198 | Y-Aib-EGT-αMeF(2F)-TSD-4Pal-SI-αMeL-LD-Orn-K(AEEA2-γGlu-C18_OH)AQ-Aib-EFIK | 935 |
| | [PEG₂₄-BEA-MalDap₂-Fc]-αMeY-LIEGSPSSGAPPPS-NH₂ | |
| 199 | Y-Aib-EGT-αMeF(2F)-TSD-4Pal-Aib-I-αMeL-LD-Orn-K(AEEA2-γGlu-C18_OH)AQ-Aib-EFIK | 935 |
| | [PEG₂₄-BEA-MalDap₂-Fc]-αMeY-LIEGSPSSGAPPPS-NH₂ | |
| 200 | Y-Aib-EGT-αMeF(2F)-TSD-4Pal-SI-αMeL-LD-Orn-K(AEEA2-γGlu-C18_OH)AQ-Aib-EFIK | 935 |
| | (AEEA6-BEA-MalDap₂-Fc)-αMeY-LIEGSPSSGAPPPS-NH₂ | |
| 201 | Y-Aib-EGT-αMeF(2F)-TSD-4Pal-Aib-I-αMeL-LD-Orn-K(AEEA2-γGlu-C18_OH)AQ-Aib-EFIK | 935 |
| | (AEEA6-BEA-MalDap₂-Fc)-αMeY-LIEGSPSSGAPPPS-NH₂ | |
| 202 | Y-Aib-EGT-αMeF(2F)-TSD-4Pal-Aib-I-αMeL-LD-Orn-K(ϵK-AEEA-γGlu-C18_OH)AQ-Aib-EFIQ- | 935 |
| | αMeY-LIEGSPSSGAPPPSK(AEEA6-BEA-MalDap₂-Fc)-NH₂ | |
| 203 | Y-Aib-EGT-αMeF(2F)-TSD-4Pal-Aib-I-αMeL-LD-Orn-K(AEEA-eK-γGlu-C18_OH)AQ-Aib-EFIQ- | 935 |
| | αMeY-LIEGSPSSGAPPPSK(AEEA6-BEA-MalDap₂-Fc)-NH₂ | |
| 204 | Y-Aib-EGT-αMeF(2F)-TSD-4Pal-Aib-I-αMeL-LD-Orn-K(ϵK2-γGlu-C18_OH)AQ-Aib-EFIE- | 935 |
| | αMeY-LIEGSPSSGAPPPSK(AEEA6-BEA-MalDap₂-Fc)-NH₂ | |
| 205 | Y-Aib-EGT-αMeF(2F)-TSD-4Pal-SI-αMeL-LD-Orn-K(ϵK2-γGlu-C18_OH)AQ-Aib-EFIE-αMeY- | 935 |
| | LIEGSPSSGAPPPSK(AEEA6-BEA-MalDap₂-Fc)-NH₂ | |
| 206 | Y-Aib-EGT-αMeF(2F)-TSDY-Aib-I-αMeL-LD-Orn-K(AEEA2-γGlu-C18_OH)AQ-Aib-EFIE-αMeY- | 935 |
| | LIEGSPSSGAPPPSK(AEEA6-BEA-MalDap₂-Fc)-NH₂ | |
| 207 | Y-Aib-EGT-αMeF(2F)-TSDY-Aib-I-αMeL-LD-Orn-K(AEEA2-γGlu-C18_OH)AQ-Aib-EFIQ-αMeY- | 935 |
| | LIEGSPSSGAPPPSK(AEEA6-BEA-MalDap₂-Fc)-NH₂ | |
| 208 | Y-Aib-EGT-αMeF(2F)-TSDY-Aib-I-αMeL-LD-Orn-K(ϵK2-γGlu-C18_OH)AQ-Aib-EFIE-αMeY- | 935 |
| | LIEGSPSSGAPPPSK(AEEA6-BEA-MalDap₂-Fc)-NH₂ | |
| 209 | Y-Aib-EGT-αMeF(2F)-TSDY-Aib-I-αMeL-LD-Orn-K(ϵK2-γGlu-C18_OH)AQ-Aib-EFIQ-αMeY- | 935 |
| | LIEGSPSSGAPPPSK(AEEA6-BEA-MalDap₂-Fc)-NH₂ | |
| 210 | Y-Aib-EGT-αMeF(2F)-TSDYSI-αMeL-LD-Orn-K(AEEA2-γGlu-C18_OH)AQ-Aib-EFIE-αMeY- | 935 |
| | LIEGSPSSGAPPPSK(AEEA6-BEA-MalDap₂-Fc)-NH₂ | |
| 211 | Y-Aib-EGT-αMeF(2F)-TSDYSI-αMeL-LD-Orn-K(AEEA2-γGlu-C18_OH)AQ-Aib-EFIQ-αMeY- | 935 |
| | LIEGSPSSGAPPPSK(AEEA6-BEA-MalDap₂-Fc)-NH₂ | |
| 212 | Y-Aib-EGT-αMeF(2F)-TSDYSI-αMeL-LD-Orn-K(ϵK2-γGlu-C18_OH)AQ-Aib-EFIE-αMeY- | 935 |
| | LIEGSPSSGAPPPSK(AEEA6-BEA-MalDap₂-Fc)-NH₂ | |
| 213 | Y-Aib-EGT-αMeF(2F)-TSDYSI-αMeL-LD-Orn-K(ϵK2-γGlu-C18_OH)AQ-Aib-EFIQ-αMeY- | 935 |
| | LIEGSPSSGAPPPSK(AEEA6-BEA-MalDap₂-Fc)-NH₂ | |
| 214 | Y-Aib-EGT-αMeF(2F)-TSD-4Pal-Aib-I-αMeL-LD-Orn-K(ϵK2-γGlu-C18-OH)AQ-Aib-EFIQ- | 935 |
| | αMeY-LIEGGGGGGGQGGGGQGGGGQK[BEA-MalDap₂-Fc]-NH₂ | |
| 215 | Y-Aib-EGT-αMeF(2F)-TSD-4Pal-SI-αMeL-LD-Orn-K(ϵK2-γGlu-C18-OH)AQ-Aib-EFIQ-αMeY- | 935 |
| | LIEGGGGGGGQGGGGQGGGGQK[BEA-MalDap₂-Fc]-NH₂ | |
| 216 | Y-Aib-EGT-αMeF(2F)-TSDY-Aib-I-αMeL-LD-Orn-K(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 217 | H-Aib-EGT-αMeF(2F)-TSD-4Pal-SI-αMeL-LD-Orn-K(AEEA2-γGlu-C18_OH)AQ-Aib-EFIE-αMeY- | 935 |
| | LIEGGGGGGGSGGGGSGGGGSK[BEA-MalDap₂-Fc]-NH₂ | |
| 218 | H-Aib-EGT-αMeF(2F)-TSD-4Pal-SI-αMeL-LD-Orn-K(ϵK2-γGlu-C18_OH)AQ-Aib-EFIE-αMeY- | 935 |
| | LIEGSPSSGAPPPS[PEG₂₄-BEA-MalDap₂-Fc]-NH₂ | |
| 219 | Y-Aib-EGT-αMeF(2F)-TSD-4Pal-Aib-I-αMeL-LD-Orn-K(AEEA2-γGlu-C18_OH)AQ-Aib-EFIE- | 935 |
| | αMeY-LIEQGGGGGGSGGGGSGGGGSK[BEA-MalDap₂-Fc]-NH₂ | |
| 220 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGGPSSGAPPPS[PEG₂₄-BEA-MalDap₂-Fc]-NH₂ | |
| 221 | Y-Aib-EGT-αMeF(2F)-TSD-4Pal-Aib-I-αMeL-LD-Orn-K(ϵK2-γGlu-C18_OH)AQ-Aib-EFIe- | 935 |
| | αMeY-LIEGGPSSGAPPPS[PEG₂₄-BEA-MalDap₂-Fc]-NH₂ | |
| 222 | Y-Aib-EGT-αMeF(2F)-TSDY-Aib-I-αMeL-LD-Orn-K(AEEA2-γGlu-C18_OH)AQ-Aib-EFIe-αMeY- | 935 |
| | LIEGGPSSGAPPPS[PEG₂₄-BEA-MalDap₂-Fc]-NH₂ | |
| 223 | Y-Aib-EGT-αMeF(2F)-TSDY-Aib-I-αMeL-LD-Orn-K(ϵK2-γGlu-C18_OH)AQ-Aib-EFIe-αMeY- | 935 |
| | LIEGGPSSGAPPPS[PEG₂₄-BEA-MalDap₂-Fc]-NH₂ | |
| 224 | Y-Aib-EGT-αMeF(2F)-TSD-4Pal-SI-αMeL-LD-Orn-K(AEEA2-γGlu-C18_OH)AQ-Aib-EFIe-αMeY- | 935 |
| | LIAGSPSSGAPPPS[PEG₂₄-BEA-MalDap₂-Fc]-NH₂ | |
| 225 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIAGSPSSGAPPPS[PEG₂₄-BEA-MalDap₂-Fc]-NH₂ | |
| 226 | Y-Aib-EGT-αMeF(2F)-TSD-4Pal-Aib-I-αMeL-LD-Orn-K(ϵK2-γGlu-C18_OH)AQ-Aib-EFIe- | 935 |
| | αMeY-LIAGSPSSGAPPPS[PEG₂₄-BEA-MalDap₂-Fc]-NH₂ | |
| 227 | Y-Aib-EGT-αMeF(2F)-TSDY-Aib-I-αMeL-LD-Orn-K(AEEA2-γGlu-C18_OH)AQ-Aib-EFIe-αMeY- | 935 |
| | LIAGSPSSGAPPPS[PEG₂₄-BEA-MalDap₂-Fc]-NH₂ | |
| 228 | Y-Aib-EGT-αMeF(2F)-TSDY-Aib-I-αMeL-LD-Orn-K(ϵK2-γGlu-C18_OH)AQ-Aib-EFIe-αMeY- | 935 |
| | LIAGSPSSGAPPPS[PEG₂₄-BEA-MalDap₂-Fc]-NH₂ | |
| 229 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIAGGPSSGAPPPS[PEG₂₄-BEA-MalDap₂-Fc]-NH₂ | |
| 230 | Y-Aib-EGT-αMeF(2F)-TSD-4Pal-SI-αMeL-LD-Orn-K(AEEA2-γGlu-C18_OH)AQ-Aib-EFIe-αMeY- | 935 |
| | LIAGGPSSGAPPPS[PEG₂₄-BEA-MalDap₂-Fc]-NH₂ | |
| 231 | Y-Aib-EGT-αMeF(2F)-TSDY-Aib-I-αMeL-LD-Orn-K(ϵK2-γGlu-C18_OH)AQ-Aib-EFIe-αMeY- | 935 |
| | LIAGGPSSGAPPPS[PEG₂₄-BEA-MalDap₂-Fc]-NH₂ | |
| 232 | Y-Aib-EGT-αMeF(2F)-TSDY-Aib-I-αMeL-LD-Orn-K(AEEA2-γGlu-C18_OH)AQ-Aib-EFIe-αMeY- | 935 |
| | LIAGGPSSGAPPPS[PEG₂₄-BEA-MalDap₂-Fc]-NH₂ | |
| 233 | Y-Aib-EGT-αMeF(2F)-TSD-4Pal-SI-αMeL-LD-Orn-K(AEEA-γGlu-C18_OH)AQ-Aib-EFIe-αMeY- | 935 |
| | LIEGSPSSGAPPPS[PEG₂₄-BEA-MalDap₂-Fc]-NH₂ | |
| 234 | Y-Aib-EGT-αMeF(2F)-TSDY-Aib-I-αMeL-LD-Orn-K(AEEA-γGlu-C18_OH)AQ-Aib-EFIe-αMeY- | 935 |
| | LIEGSPSSGAPPPS[PEG₂₄-BEA-MalDap₂-Fc]-NH₂ | |
| 235 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LVEGSPSSGAPPPS[PEG₂₄-BEA-MalDap₂-Fc]-NH₂ | |
| 236 | Y-Aib-EGT-αMeF(2F)-TSD-4Pal-SI-αMeL-LD-Orn-K(AEEA2-γGlu-C18_OH)AQ-Aib-EFIE-αMeY- | 935 |
| | LIAGSPSSGAPPPS[PEG₂₄-BEA-MalDap₂-Fc]-NH₂ | |
| 237 | Y-Aib-EGT-αMeF(2F)-TSD-4Pal-SI-αMeL-LD-Orn-K(ϵK2-γGlu-C18_OH)AQ-Aib-EFIE-αMeY- | 935 |
| | LIAGSPSSGAPPPS[PEG₂₄-BEA-MalDap₂-Fc]-NH₂ | |
| 238 | Y-Aib-EGT-αMeF(2F)-TSDY-Aib-I-αMeL-LD-Orn-K(AEEA2-γGlu-C18_OH)AQ-Aib-EFIE-αMeY- | 935 |
| | LIAGSPSSGAPPPS[PEG₂₄-BEA-MalDap₂-Fc]-NH₂ | |
| 239 | Y-Aib-EGT-αMeF(2F)-TSDY-Aib-I-αMeL-LD-Orn-K(ϵK2-γGlu-C18_OH)AQ-Aib-EFIE-αMeY- | 935 |
| | LIAGSPSSGAPPPS-NH₂ | |
| 240 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPS-NH₂ | |
| 241 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPS-NH₂ | |
| 242 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPS-NH₂ | |
| 243 | Y-Aib-EGT-αMeF(2F)-TSD-4Pal-SI-αMeL-LDEK(Ac)AQ-Aib-EFIe-αMeY-LIEGSPSSGAPPPS-NH₂ | 935 |
| 244 | Y-Aib-EGT-αMeF(2F)-TSD-4Pal-SI-αMeL-LDEQAQ-Aib-EFIe-αMeY-LIEGSPSSGAPPPS[PEG₂₄- | 935 |
| | BEA-MalDap₂-Fc]-NH₂ | |
| 245 | Y-Aib-EGT-αMeF(2F)-TSD-4Pal-SI-αMeL-LDEQAQ-Aib-EFIe-αMeY-LIEGSPSSGAPPPSK(PEG₂₄- | 936 |
| | BEA-MalDap₂-Fc KRQ)-NH₂ | |
| 246 | Y-Aib-EGT-αMeF(2F)-TSD-4Pal-SI-αMeL-LD-Orn-K(AEEA2-γGlu-C20_OH)AQ-Aib-EFIe-αMeY- | 935 |
| | LIEGSPSSGAPPPS[PEG₂₄-BEA-MalDap₂-Fc]-NH₂ | |
| 247 | Y-Aib-EGT-αMeF(2F)-TSD-4Pal-SI-αMeL-LD-Orn-K(AEEA2-γGlu-C16_OH)AQ-Aib-EFIe-αMeY- | 935 |
| | LIEGSPSSGAPPPS[PEG₂₄-BEA-MalDap₂-Fc]-NH₂ | |
| 248 | Y-Aib-EGT-αMeF(2F)-TSD-4Pal-SI-αMeL-LDEK(AEEA2-γGlu-C16_OH)AQ-Aib-EFIe-αMeY- | 935 |
| | LIEGSPSSGAPPPS[PEG₂₄-BEA-MalDap₂-Fc]-NH₂ | |
| 249 | Y-Aib-EGT-αMeF(2F)-TSD-4Pal-SI-αMeL-LD-Orn-K(ϵK2-γGlu-C20_OH)AQ-Aib-EFIe-αMeY- | 935 |
| | LIEGSPSSGAPPPS[PEG₂₄-BEA-MalDap₂-Fc]-NH₂ | |
| 250 | Y-Aib-EGT-αMeF(2F)-TSD-4Pal-SI-αMeL-LDEK(ϵK2-γGlu-C20_OH)AQ-Aib-EFIe-αMeY- | 935 |
| | LIEGSPSSGAPPPS[PEG₂₄-BEA-MalDap₂-Fc]-NH₂ | |
| 251 | Y-Aib-EGT-αMeF(2F)-TSD-4Pal-SI-αMeL-LD-Orn-K(AEEA2-γGlu-C16_OH)AQ-Aib-EFIE-αMeY- | 935 |
| | LIEGGGGGGGSGGGGSGGGGSK[BEA-MalDap₂-Fc]-NH₂ | |
| 252 | Y-Aib-EGT-αMeF(2F)-TSD-4Pal-SI-αMeL-LDEK(γGlu-AEEA-ϵK-C20_OH)AQ-Aib-EFIe-αMeY- | 935 |
| | LIEGSPSSGAPPPS-[PEG₂₄-BEA-MalDap₂-Fc]NH₂ | |
| 253 | Y-Aib-EGT-αMeF(2F)-TSD-4Pal-Aib-I-αMeL-LD-Orn-K(AEEA2-γGlu-C16_OH)AQ-Aib-EFIE- | 935 |
| | αMeY-LIEGGGGGGGSGGGGSGGGGSK[BEA-MalDap₂-Fc)-NH₂ | |
| 254 | Y-Aib-EGT-αMeF(2F)-TSD-4Pal-Aib-I-αMeL-LD-Orn-K(AEEA2-γGlu-C16_OH)AQ-Aib-EFIE- | 935 |
| | αMeY-LIEGGPSSGK(PEG₂₄-BEA-MalDap₂-Fc)-NH₂ | |
| 255 | Y-Aib-EGT-αMeF(2F)-TSD-4Pal-Aib-I-αMeL-LD-Orn-K(AEEA2-γGlu-C16_OH)AQ-Aib-EFIE- | 935 |
| | αMeY-LIEGGG-PEG₂₄-K[BEA-MalDap₂-Fc]-NH₂ | |
| 256 | Y-Aib-EGT-αMeF(2F)-TSD-4Pal-Aib-I-αMeL-LD-Orn-K(AEEA2-γGlu-C16_OH)AQ-Aib-EFIE- | 935 |
| | αMeY-LIEGGGK(PEG₂₄-BEA-MalDap₂-Fc)-NH₂ | |
| 257 | Y-Aib-EGT-αMeF(2F)-TSD-4Pal-Aib-I-αMeL-LDEK(ϵK2-γGlu-C16_OH)AQ-Aib-EFIE-αMeY- | 935 |
| | LIEGGPSSGK[PEG₂₄-BEA-MalDap₂-Fc]-NH₂ | |
| 258 | Y-Aib-EGT-αMeF(2F)-TSD-4Pal-Aib-I-αMeL-LDEK(ϵK2-γGlu-C16_OH)AQ-Aib-EFIE-αMeY- | 935 |
| | LIEGGG-PEG₂₄-K[BEA-MalDap₂-Fc]-NH₂ | |
| 259 | Y-Aib-EGT-αMeF(2F)-TSD-4Pal-Aib-I-αMeL-LDEK(ϵK2-γGlu-C16_OH)AQ-Aib-EFIE-αMeY- | 935 |
| | LIEGGGK(PEG₂₄-BEA-MalDap₂-Fc)-NH₂ | |
| 260 | Y-Aib-EGT-αMeF(2F)-TSD-4Pal-SI-αMeL-LDEK(AEEA2-γGlu-C16_OH)AQ-Aib-EFIE-αMeY- | 935 |
| | LIEGGGGGGGSGGGGSGGGGSK[BEA-MalDap₂-Fc]-NH₂ | |
| 261 | Y-Aib-EGT-αMeF(2F)-TSD-4Pal-SI-αMeL-LDEK(AEEA2-γGlu-C16)AQ-Aib-EFIE-αMeY- | 935 |
| | LIEGGGGGGGSGGGGSGGGGSK[BEA-MalDap₂-Fc]-NH₂ | |
| 262 | Y-Aib-EGT-αMeF(2F)-TSD-4Pal-SI-αMeL-LDEK(AEEA2-γGlu-C18_OH)AQ-Aib-EFIE-αMeY- | 935 |
| | LIEGGGGGGGSGGGGSGGGGSK[BEA-MalDap₂-Fc]-NH₂ (Duplicated from above) | |
| 263 | Y-Aib-EGT-αMeF(2F)-TSD-4Pal-SI-αMeL-LDEK(AEEA2-γGlu-C18_OH)AQ-Aib-EFIe-αMeY- | 936 |
| | LIEGSPSSGAPPPSK((AEEA4-GKAAAEKAAAEKAAAE-[BEAMalDap₂-Fc KRQ]NH₂ | |
| 264 | Y-Aib-EGT-αMeF(2F)-TSDV-Aib-I-αMeL-LD-EK(AEEA2-γGlu-C18_OH)AQ-Aib-EFIe-αMeY- | 936 |
| | LIEGSPSSGAPPPSK(PEG₂₄-BEA-MalDap₂-Fc KRQ)-NH₂ | |
| 265 | Y-Aib-EGT-αMeF(2F)-TSD-4Pal-SI-αMeL-LDEK(AEEA2-γGlu-C18_OH)AQ-Aib-EFIK(PEG₂₄- | 936 |
| | BEA-MalDap₂-Fc KRQ)-αMeY-LIEGSPSSGAPPPSGS-NH₂ | |
| 266 | Y-Aib-EGT-αMeF(2F)-TSD-4Pal-SI-αMeL-LDEK(AEEA2-γGlu-C18_OH)AQ-Aib-EFIK(PEG₂₄- | 936 |
| | BEA-MalDap₂-Fc KRQ)-αMeY-LIEGSPSSGAPPPSGS-NH₂ | |
| 267 | Y-Aib-EGT-αMeF(2F)-TSD-4Pal-SI-αMeL-LDEK(AEEA2-γGlu-C18_OH)AQ-Aib-EFIe-αMeY- | 936 |
| | LIEGGGGGGGSGGGGSGAPAPAPAPAPAPK(BEA-MalDap₂-Fc KRQ)-NH₂ | |
| 268 | Y-Aib-EGT-αMeF(2F)-TSD-4Pal-SI-αMeL-LD-Orn-K(ϵK2-γGlu-C18_OH)AQ-Aib-EFIe-αMeY- | 936 |
| | LIEGSPSSGAPPPS-K(PEG₂₄-BEA-MalDap₂-Fc KRQ)-NH₂ | |
| 269 | Y-Aib-EGT-αMeF(2F)-TSD-V-Aib-I-αMeL-LD-Orn-K(AEEA2-γGlu-C18_OH)AQ-Aib-EFIe-αMeY- | 936 |
| | LIEGGGGGGGSGGGGGGGGSK[BEA-MalDap₂-Fc KRQ]-NH₂ | |
| 270 | Y-Aib-EGT-αMeF(2F)-TSD-4Pal-SI-αMeL-LD-Orn-K(AEEA2-γGlu-C18_OH)AQ-Aib-EFIE-αMeY- | 936 |
| | LIEGGGGGGGSGGGGSGGGGSK[BEA-MalDap₂-Fc KRQ]-NH₂ | |
| 271 | Y-Aib-EGT-αMeF(2F)-TSD-4Pal-SI-αMeL-LDEK(AEEA2-γGlu-C18_OH)AQ-Aib-EFIe-αMeY- | 936 |
| | LIEGGGGGGGSGGGGSGGGGSGAPAPAPAPAPAPK[BEA-MalDap₂-Fc KRQ]-NH₂ | |
| 272 | Y-Aib-EGT-αMeF(2F)-TSD-4Pal-SI-αMeL-LDEK(γGlu-AEEA-ϵK-C20_OH)AQ-Aib-EFIe-αMeY- | 936 |
| | LIEGSPSSGAPPPS-K(PEG₂₄-BEA-MalDap₂-Fc KRQ)-NH₂ | |
| 273 | Y-Aib-EGT-αMeF(2F)-TSD-4Pal-Aib-I-αMeL-LDEK(AEEA2-γGlu-C18_OH)AQ-Aib-EFIE-αMeY- | 936 |
| | LIEGGGGGGGSGGGGSGGGGSGAPAPAPAPAPAPK[BEA-MalDap₂-Fc KRQ]-NH₂ | |
| 274 | Y-Aib-EGT-αMeF(2F)-TSD-4Pal-Aib-I-αMeL-LDEK(AEEA2-γGlu-C18_OH)AQ-Aib-EFIE-αMeY- | 936 |
| | LIEGGGGGGGSGGGGGGGGSGEAAAKEAAAKEAAAKK[BEA-MalDap₂-Fc KRQ]-NH₂ | |
| 275 | Y-Aib-EGT-αMeF(2F)-TSD-4Pal-SI-αMeL-LEEK(AEEA2-γGlu-C18_OH)AQ-Aib-EFIE-αMeY- | 936 |
| | LIEGGGGGGGSGGGGSGGGGSGAPAPAPAPAPAPK[BEA-MalDap₂-Fc KRQ]-NH₂ | |
| 276 | Y-Aib-EGT-αMeF(2F)-TSD-4Pal-SI-αMeL-LEEK(AEEA2-γGlu-C18_OH)AQ-Aib-EFIE-αMeY- | 936 |
| | LIEGGGGGGGSGEAAAKEAAAKEAAAKK[BEA-MalDap₂-Fc KRQ]-NH₂ | |
| 277 | Y-Aib-EGT-αMeF(2F)-TSD-4Pal-SI-αMeL-LEEK(AEEA2-γGlu-C18_OH)AQ-Aib-EFIE-αMeY- | 936 |
| | LIEGGGGGGGSGGGGSGGGGSGEPEPEPEPEPEPK[BEA-MalDap₂-Fc KRQ]-NH₂ | |
| 278 | Y-Aib-EGT-αMeF(2F)-TSD-4Pal-SI-αMeL-LEEK(AEEA2-γGlu-C18_OH)AQ-Aib-EFIE-αMeY- | 936 |
| | LIEGGGGGGGQGGGGQGGGGQGEPEPEPEPEPEPK[BEA-MalDap₂-Fc KRQ]-NH₂ | |
| 279 | Y-Aib-EGT-αMeF(2F)-TSD-4Pal-SI-αMeL-LE-Orn-K(AEEA2-γGlu-C18_OH)AQ-Aib-EFIE-αMeY- | 936 |
| | LIEGGGGGGGSGEPEPEPEPEPEPK[BEA-MalDap₂-Fc KRQ]-NH₂ | |
| 280 | Y-Aib-EGT-αMeF(2F)-TSD-4Pal-SI-αMeL-LE-Orn-K(AEEA2-γGlu-C18_OH)AQ-Aib-EFIE-αMeY- | 936 |
| | LIEGGGGGGGSGGGGSGGGGSGEPEPEPEPEPEPK[BEA-MalDap₂-Fc KRQ]-NH₂ | |
| 281 | Y-Aib-EGT-αMeF(2F)-TSD-4Pal-SI-αMeL-LE-Orn-K(AEEA2-γGlu-C18_OH)AQ-Aib-EFIE-αMeY- | 936 |
| | LIEGSPSSGAPPPS-K(AEEA6-GPAPAPAPAPAPA-[BEAMalDap₂-Fc KRQ]-NH₂ | |
| 282 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGGGGGGGSGGGGSGAPAPAPAPAPAPK[BEA-MalDap₂-Fc KRQ]-NH₂ | |
| 283 | Y-Aib-EGT-αMeF(2F)-TSD-4Pal-SI-αMeL-LE-Orn-K(AEEA2-γGlu-C18_OH)AQ-Aib-EFIE-αMeY- | 936 |
| | LIEGGGGGGGSGGGGSGAPAPAPAPAPAPK[BEA-MalDap₂-Fc KRQ]-NH₂ | |
| 284 | Y-Aib-EGT-αMeF(2F)-TSD-4Pal-Aib-I-αMeL-LD-Orn-K(AEEA2-γGlu-C18_OH)AQ-Aib-EFIe- | 936 |
| | αMeY-LIEGGGGGGGSGGGGSGAPAPAPAPAPAPK[BEA-MalDap₂-Fc KRQ]-NH₂ | |
| 285 | Y-Aib-EGT-αMeF(2F)-TSD-4Pal-SI-αMeL-LD-Orn-K(AEEA2-γGlu-C18_OH)AQ-Aib- | 936 |
| | EFIKGGGGSGGGGSGAPAPAPAPAPAPG-[BEA-MalDap)₂-Fc KRQ]-αMeY-LIEGSPSSGAPPPSGS- | |
| | NH₂ | |
| 286 | Y-Aib-EGT-αMeF(2F)-TSD-4Pal-Aib-I-αMeL-LD-Orn-K(AEEA2-γGlu-C18_OH)AQ-Aib- | 936 |
| | EFIK(GGGGSGGGGSGAPAPAPAPAPAPG-BEA-MalDap₂-Fc KRQ)-αMeY-LIEGSPSSGAPPPSGS- | |
| | NH₂ | |
| 287 | Y-Aib-EGT-αMeF(2F)-TSD-4Pal-Aib-I-αMeL-LD-Orn-K(ϵK2-γGlu-C18_OH)AQ-Aib-EFIE- | 936 |
| | αMeY-LIEGGPSSGK(PEG₂₄-BEA-MalDap₂-Fc KRQ)-NH₂ | |
| 288 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGGPSSGK(PEG₂₄-BEA-MalDap₂-Fc KRQ)-NH₂ | |
| 289 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGGG-PEG₂₄-K[BEA-MalDap₂-Fc KRQ]-NH₂ | |
| 290 | Y-Aib-EGT-αMeF(2F)-TSD-4Pal-Aib-I-αMeL-LD-Orn-K(AEEA2-γGlu-C16_OH)AQ-Aib-EFIE- | 936 |
| | αMeY-LIEGGGGGGGSGGGGSGGGGSK[BEA-MalDap₂-Fc KRQ]-NH₂ | |
| 291 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGSPSSGAPPPSK(PEG₂₄-BEA-MalDap₂-Fc KRQ)-NH₂ | |
| 292 | Y-Aib-EGT-αMeF(2F)-TSD-4Pal-Aib-I-αMeL-LD-Orn-K(ϵK2-γGlu-C18_OH)AQ-Aib-EFIE- | 936 |
| | αMeY-LIEGSPSSGAPPPSK[PEG₂₄-BEA-MalDap₂-Fc KRQ]-NH₂ | |
| 293 | Y-Aib-EGT-αMeF(2F)-TSD-4Pal-Aib-I-αMeL-LDEK(ϵK2-γGlu-C18_OH)AQ-Aib-EFIE-αMeY- | 936 |
| | LIEGSPSSGAPPPSK[PEG₂₄-BEA-MalDap₂-Fc KRQ]-NH₂ | |
| 294 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGGPSSGAPPPSK[PEG₂₄-BEA-MalDap₂-Fc KRQ]-NH₂ | |
| 295 | Y-Aib-EGT-αMeF(2F)-TSD-4Pal-Aib-I-αMeL-LD-Orn-K(ϵK2-γGlu-C18_OH)AQ-Aib-EFIE- | 936 |
| | αMeY-LIEGGPSSGAPPPSK(PEG₂₄-BEA-MalDap₂-Fc KRQ)-NH₂ | |
| 296 | Y-Aib-EGT-αMeF(2F)-TSD-4Pal-Aib-I-αMeL-LDEK(ϵK2-γGlu-C18_OH)AQ-Aib-EFIE-αMeY- | 936 |
| | LIEGGPSSGAPPPSK(PEG₂₄-BEA-MalDap₂-Fc KRQ)-NH₂ | |
| 297 | Y-Aib-EGT-αMeF(2F)-TSD-4Pal-Aib-I-αMeL-LD-Orn-K(ϵK2-γGlu-C18_OH)AQ-Aib-EFIe- | 936 |
| | αMeY-LIEGSPSSGAPPPSK(PEG₂₄-BEA-MalDap₂ Fc KRQ)-NH₂ | |
| 298 | Y-Aib-EGT-αMeF(2F)-TSD-4Pal-Aib-I-αMeL-LD-Orn-K(ϵK2-γGlu-C18_OH)AQ-Aib-EFIe- | 936 |
| | αMeY-LIEGGGK(PEG₂₄-BEA-MalDap₂ Fc KRQ)-NH₂ | |
| 299 | Y-Aib-EGT-αMeF(2F)-TSD-4Pal-Aib-I-αMeL-LD-Orn-K(ϵK2-γGlu-C18_OH)AQ-Aib-EFIE- | 936 |
| | αMeY-LIEGG-Orn-SSGAPPPSK(PEG₂₄-BEA-MalDap₂ Fc KRQ)-NH₂ | |
| 300 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGG-Orn-SSGAPPPSK(PEG₂₄-BEA-MalDap₂ Fc KRQ)-NH₂ | |
| 301 | Y-Aib-EGT-αMeF(2F)-TSD-4Pal-Aib-I-αMeL-LD-Orn-K(ϵK2-γGlu-C18_OH)AQ-Aib-EFIE- | 936 |
| | αMeY-LIEGG-Orn-K(PEG₂₄-BEA-MalDap₂ Fc KRQ)-NH₂ | |
| 302 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGG-Orn-K(PEG₂₄-BEA-MalDap₂ Fc KRQ)-NH₂ | |
| 303 | Y-Aib-EGT-αMeF(2F)-TSD-4Pal-Aib-I-αMeL-LD-Orn-K(ϵK2-γGlu-C18_OH)AQ-Aib-Orn-FIE- | 936 |
| | αMeY-LIEGSPSSGAPPPSK(PEG₂₄-BEA-MalDap₂ Fc KRQ)-NH₂ | |
| 304 | Y-Aib-EGT-αMeF(2F)-TSD-4Pal-Aib-I-αMeL-LD-Orn-K(AEEA2-γGlu-C18_OH)AQ-Aib-Orn- | 936 |
| | FIE-αMeY-LIEGSPSSGAPPPSK(PEG₂₄-BEA-MalDap₂ Fc KRQ)-NH₂ | |
| 305 | Y-Aib-EGT-αMeF(2F)-TSD-4Pal-Aib-I-αMeL-LD-Orn-K(AEEA2-γGlu-C18_OH)AQ-Aib-EFIQ- | 936 |
| | αMeY-LIEGSPSSGAPPPSK(PEG₂₄-BEA-MalDap₂ Fc KRQ)-NH₂ | |
| 306 | Y-Aib-EGT-αMeF(2F)-TSD-4Pal-Aib-I-αMeL-LD-Orn-K(ϵK2-γGlu-C18_OH)AQ-Aib-EFIQ- | 936 |
| | αMeY-LIEGSPSSGAPPPSK(PEG₂₄-BEA-MalDap₂ Fc KRQ)-NH₂ | |
| 307 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGSPSSGAPPPSK(AEEA6-GPAPAPAPAPAPA-BEA-MalDap₂-Fc KRQ)-NH₂ | |
| 308 | Y-Aib-EGT-αMeF(2F)-TSD-4Pal-SI-αMeL-LD-Orn-K(AEEA2-γGlu-C18_OH)AQ-Aib-EFIE-αMeY- | 936 |
| | LIEGSPSSGAPPPSK(AEEA6-GPAPAPAPAPAPA-BEA-MalDap₂-Fc KRQ)-NH₂ | |
| 309 | Y-Aib-EGT-αMeF(2F)-TSD-4Pal-SI-αMeL-LD-Orn-K(ϵK2-γGlu-C18 OH)AQ-Aib-EFIE-αMeY- | 936 |
| | LIEGSPSSGAPPPSK(AEEA6-GPAPAPAPAPAPA-BEA-MalDap₂ Fc KRQ)-NH₂ | |
| 310 | Y-Aib-EGT-αMeF(2F)-TSD-4Pal-SI-αMeL-LD-Orn-K(ϵK2-γGlu-C18_OH)AQ-Aib-EFIe-αMeY- | 936 |
| | LIEGSPSSGAPPPSK(AEEA6-GPAPAPAPAPAPA-BEA-MalDap₂-Fc KRQ)-NH₂ | |
| 311 | Y-Aib-EGT-αMeF(2F)-TSD-4Pal-Aib-I-αMeL-LD-Orn-K(AEEA2-γGlu-C18_OH)AQ-Aib-EFIE- | 936 |
| | αMeY-LIEGSPSSGAPPPSK(AEEA6-BEA-MalDap₂-Fc KRQ)-NH₂ | |
| 312 | Y-Aib-EGT-αMeF(2F)-TSD-4Pal-SI-αMeL-LD-Orn-K(ϵK2-γGlu-C18_OH)AQ-Aib-EFIe-αMeY- | 941 |
| | LIEGSPSSGAPPPSK(PEG₂₄-BEA-MalDap₂-Fc IG KRQ)-NH₂ | |
| 313 | Y-Aib-EGT-αMeF(2F)-TSD-4Pal-SI-αMeL-LE-Orn-K(AEEA2-γGlu-C18_OH)AQ-Aib-EFIE-αMeY- | 941 |
| | LIEGGGGGGGSGGGGSGAPAPAPAPAPAPK[BEA-MalDap₂-Fc IG KRQ]-NH₂ | |
| 314 | Y-Aib-EGT-αMeF(2F)-TSD-4Pal-Aib-I-αMeL-LD-Orn-K(AEEA2-γGlu-C18_OH)AQ-Aib-EFIQ- | 941 |
| | αMeY-LIEGSPSSGAPPPSK(PEG₂₄-BEA-MalDap₂ Fc IG KRQ)-NH₂ | |
| 315 | Y-Aib-EGT-αMeF(2F)-TSD-4Pal-Aib-I-αMeL-LD-Orn-K(ϵK2-γGlu-C18_OH)AQ-Aib-EFIQ- | 936 |
| | αMeY-LIEGSPSSGAPPPSK(PEG₂₄-BEA-MalDap₂ Fc KRQ)-NH₂ | |
| 316 | Y-Aib-EGT-αMeF(2F)-TSD-4Pal-Aib-I-αMeL-LD-Orn-K(AEEA2-γGlu-C18_OH)AQ-Aib-EFIE- | 941 |
| | αMeY-LIEGSPSSGAPPPSK(AEEA6-BEA-MalDap₂-Fc IG KRQ)-NH₂ | |
| 317 | Y-Aib-EGT-αMeF(2F)-TSD-4Pal-SI-αMeL-LD-Orn-K(AEEA2-γGlu-C18_OH)AQ-Aib-EFIQ-αMeY- | 941 |
| | LIEGSPSSGAPPPSK(PEG₂₄-BEA-MalDap₂-Fc IG KRQ)-NH₂ | |
| 318 | Y-Aib-EGT-αMeF(2F)-TSDY-Aib-I-αMeL-LD-Orn-K(AEEA2-γGlu-C18_OH)AQ-Aib-EFIe-αMeY- | 941 |
| | LIEGSPSSGAPPPSK(PEG₂₄-BEA-MalDap₂-Fc IG KRQ)-NH₂ | |
| 319 | Y-Aib-EGT-αMeF(2F)-TSDY-Aib-I-αMeL-LD-Orn-K(AEEA2-γGlu-C18_OH)AQ-Aib-EFIE-αMeY- | 935 |
| | LIEGSPSSGAPPPSK[PEG₂₄-BEA-MalDap₂-Fc]-NH₂ | |
| 320 | Y-Aib-EGT-αMeF(2F)-TSD-4Pal-Aib-I-αMeL-LD-Orn-K(AEEA2-γGlu-C18_OH)AQ-Aib-EFIQ- | 941 |
| | αMeY-LIE-Aib-SPSSGAPPPSK(PEG₂₄-BEA-MalDap₂-Fc IG KRQ)-NH₂ | |
| 321 | Y-Aib-EGT-αMeF(2F)-TSD-4Pal-Aib-I-αMeL-LD-Orn-K(ϵK2-γGlu-C18_OH)AQ-Aib-EFIQ- | 941 |
| | αMeY-LIE-Aib-SPSSGAPPPSK(PEG₂₄-BEA-MalDap₂-Fc IG KRQ)-NH₂ | |
| 322 | Y-Aib-EGT-αMeF(2F)-TSD-4Pal-SI-αMeL-LD-Orn-K(AEEA2-γGlu-C18_OH)AQ-Aib-EFIQ-αMeY- | 941 |
| | LIEGSPSSGAPPPSK(AEEA6-GPAPAPAPAPAPA-BEA-MalDap₂-Fc IG KRQ)-NH₂ | |
| 323 | Y-Aib-EGT-αMeF(2F)-TSD-4Pal-SI-αMeL-LD-Orn-K(ϵK2-γGlu-C18_OH)AQ-Aib-EFIQ-αMeY- | 941 |
| | LIEGSPSSGAPPPSK(AEEA6-GPAPAPAPAPAPA-BEA-MalDap₂-Fc IG KRQ)-NH₂ | |
| 324 | Y-Aib-EGT-αMeF(2F)-TSD-4Pal-SI-αMeL-LD-Orn-K(AEEA2-γGlu-C18_OH)AQ-Aib-EFIQ-αMeY- | 941 |
| | LIEGSPSSGAPPPSK(PEG₂₄-BEA-MalDap₂-Fc IG KRQ)-NH₂ | |
| 325 | Y-Aib-EGT-αMeF(2F)-TSD-4Pal-SI-αMeL-LD-Orn-K(ϵK2-γGlu-C18_OH)AQ-Aib-EFIQ-αMeY- | 941 |
| | LIEGSPSSGAPPPSK(PEG₂₄-BEA-MalDap₂-Fc IG KRQ)-NH₂ | |
| 326 | Y-Aib-EGT-αMeF(2F)-TSD-4Pal-SI-αMeL-LD-Orn-K(AEEA2-γGlu-C18_OH)AQ-Aib-EFIQ-αMeY- | 941 |
| | LIEGSPSSGAPPPSK(AEEA6-BEA-MalDap₂-Fc IG KRQ)-NH₂ | |
| 327 | Y-Aib-EGT-αMeF(2F)-TSD-4Pal-SI-αMeL-LD-Orn-K(ϵK2-γGlu-C18_OH)AQ-Aib-EFIQ-αMeY- | 941 |
| | LIEGSPSSGAPPPSK(AEEA6-BEA-MalDap₂-Fc IG KRQ)-NH₂ | |
| 328 | Y-Aib-EGT-αMeF(2F)-TSD-4Pal-SI-αMeL-LD-Orn-K(ϵK2-γGlu-C18_OH)AQ-Aib-EFIe-αMeY- | 942 |
| | LIEGSPSSGAPPPS-K(PEG₂₄-BEA-MalDap₂-Fc N-Cys)-NH₂ | |
| 329 | Y-Aib-EGT-αMeF(2F)-TSD-4Pal-SI-αMeL-LD-Orn-K(ϵK2-γGlu-C18_OH)AQ-Aib-EFIe-αMeY- | 942 |
| | LIEGSPSSGAPPPS-K(PEG₂₄-BEA-MalDap₂-Fc N-Cys KRQ)-NH₂ | |
| 330 | Y-Aib-EGT-αMeF(2F)-TSD-4Pal-Aib-I-αMeL-LD-Orn-K(AEEA2-γGlu-C18_OH)AQ-Aib-EFIe- | 935 |
| | αMeY-LIEGGGGGGGSGGGGSGAPAPAPAPAPAPK[BEA-MalDap₂-Fc KRQ]-NH₂ | |
| 331 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPS-K(PEG₂₄-BEA-MalDap₂-Fc KRQ)-NH₂ | |
| 332 | Y-Aib-QGT-αMeF(2F)-TSD-4Pal-SI-αMeL-LD-Orn-K(AEEA2-γGlu-C18_OH)AQ-αMe4Pal-Orn- | 935 |
| | FIe-αMeY-LIEGSPSSGAPPPSK[PEG₂₄-BEA-MalDap₂-Fc]-NH₂ | |
| 333 | Y-Aib-QGT-αMeF(2F)-TSD-4Pal-SI-αMeL-LD-Orn-K(AEEA2-γGlu-C18_OH)AQ-αMe4Pal-Orn- | 935 |
| | FIE-αMeY-LIEGSPSSGAPPPSK[PEG₂₄-BEA-MalDap₂-Fc]-NH₂ | |
| 334 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIe-αMeY-LIEGSPSSGAPPPSK[PEG₂₄-BEA-MalDap₂-Fc]-NH₂ | |
| 335 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIE-αMeY-LIEGSPSSGAPPPSK[PEG₂₄-BEA-MalDap₂-Fc]-NH₂ | |
| 336 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGSPSSGAPPPSK[PEG₂₄-BEA-MalDap₂-Fc]-NH₂ | |
| 337 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIE-αMeY-LIEGSPSSGAPPPSK[PEG₂₄-BEA-MalDap₂-Fc]-NH₂ | |
| 338 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIE-αMeY-LIEGGGGGGGSGGGGSGAPAPAPAPAPAPK[BEA-MalDap₂-Fc]-NH₂ | |
| 339 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGGGGGGGSGGGGSGAPAPAPAPAPAPK[BEA-MalDap₂-Fc]-NH₂ | |
| 340 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIE-αMeY-LIEGGPSSGAPPPSGK[PEG₂₄-BEA-MalDap₂-Fc]-NH₂ | |
| 341 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGGPSSGAPPPSGK[PEG₂₄-BEA-MalDap₂-Fc]-NH₂ | |
| 342 | Y-Aib-QGT-αMeF(2F)-TSD-4Pal-αMeS-I-αMeL-LD-Orn-K(ϵK2-γGlu-C18_OH)AQ-αMe4Pal-Orn- | 935 |
| | FIE-αMeY-LIEGGPSSGAPPPSGK[PEG₂₄-BEA-MalDap₂-Fc]-NH₂ | |
| 343 | Y-Aib-QGT-αMeF(2F)-TSDY-αMeS-I-αMeL-LD-Orn-K(ϵK2-γGlu-C18_OH)AQ-αMe4Pal-Orn-FIe- | 935 |
| | αMeY-LIEGGPSSGAPPPSGK[PEG₂₄-BEA-MalDap₂-Fc]-NH₂ | |
| 344 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIK[PEG₂₄-BEA-MalDap₂-Fc]-αMeY-LIEGGPSSGAPPPSGG-NH₂ | |
| 345 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIK[PEG₂₄-BEA-MalDap₂-Fc]-αMeY-LIEGGPSSGAPPPSGG-NH₂ | |
| 346 | Y-Aib-QGT-αMeF(2F)-TSD-4Pal-αMeS-I-αMeL-LD-Orn-K(ϵK2-γGlu-C18_OH)AQ-αMe4Pal-Orn- | 935 |
| | FIK[PEG₂₄-BEA-MalDap₂-Fc]-αMeY-LIEGGPSSGAPPPSGG-NH₂ | |
| 347 | Y-Aib-QGT-αMeF(2F)-TSDY-αMeS-I-αMeL-LD-Orn-K(ϵK2-γGlu-C18_OH)AQ-αMe4Pal-Orn- | 935 |
| | FIK[PEG₂₄-BEA-MalDap₂-Fc]-αMeY-LIEGGPSSGAPPPSGG-NH₂ | |
| 348 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIE-αMeY-LIEGGPSSGK[PEG₂₄-BEA-MalDap₂-Fc]-NH₂ | |
| 349 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGGPSSGK[PEG₂₄-BEA-MalDap₂-Fc]-NH₂ | |
| 350 | Y-Aib-QGT-αMeF(2F)-TSD-4Pal-αMeS-I-αMeL-LD-Orn-K(ϵK2-γGlu-C18_OH)AQ-αMe4Pal- | 935 |
| | Orn-FIE-αMeY-LIEGGPSSGK[PEG₂₄-BEA-MalDap₂-Fc]-NH₂ | |
| 351 | Y-Aib-QGT-αMeF(2F)-TSDY-αMeS-I-αMeL-LD-Orn-K(ϵK2-γGlu-C18_OH)AQ-αMe4Pal-Orn- | 935 |
| | FIe-αMeY-LIEGGPSSGK[PEG₂₄-BEA-MalDap₂-Fc]-NH₂ | |
| 352 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIE-αMeY-LIEGGGK[PEG₂₄-BEA-MalDap₂-Fc]-NH₂ | |
| 353 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGGGK[PEG₂₄-BEA-MalDap₂-Fc]-NH₂ | |
| 354 | Y-Aib-QGT-αMeF(2F)-TSD-4Pal-αMeS-I-αMeL-LD-Orn-K(ϵK2-γGlu-C18_OH)AQ-αMe4Pal-Orn- | 935 |
| | FIE-αMeY-LIEGGGK[PEG₂₄-BEA-MalDap₂-Fc]-NH₂ | |
| 355 | Y-Aib-QGT-αMeF(2F)-TSDY-αMeS-I-αMeL-LD-Orn-K(ϵK2-γGlu-C18_OH)AQ-αMe4Pal-Orn-FIe- | 935 |
| | αMeY-LIEGGGK[PEG₂₄-BEA-MalDap₂-Fc]-NH₂ | |
| 356 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIE-αMeY-LIEGGGGGGGQGGGGQGGGGQK[BEA-MalDap₂-Fc]-NH₂ | |
| 357 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGGGGGGGQGGGGQGGGGQK[BEA-MalDap₂-Fc]-NH₂ | |
| 358 | Y-Aib-QGT-αMeF(2F)-TSD-4Pal-αMeS-I-αMeL-LD-Orn-K(ϵK2-γGlu-C18_OH)AQ-αMe4Pal-Orn- | 935 |
| | FIE-αMeY-LIEGSPSSGAPPPSK(PEG₂₄-BEA-MalDap₂ Fc)-NH₂ | |
| 359 | Y-Aib-QGT-αMeF(2F)-TSDY-αMeS-I-αMeL-LD-Orn-K(ϵK2-γGlu-C18_OH)AQ-αMe4Pal-Orn-FIe- | 935 |
| | αMeY-LIEGSPSSGAPPPSK(PEG₂₄-BEA-MalDap₂ Fc)-NH₂ | |
| 360 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIe-αMeY-LIEGSPSSGK(PEG₂₄-BEA-MalDap₂ Fc)-NH₂ | |
| 361 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIe-αMeY-LIEGGGGGGGSGGGGSGGGGSK(BEA-MalDap₂ Fc)-NH₂ | |
| 362 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIe-αMeY-LIEGGGGGGGSGGGGSGAPAPAPAPAPAPK(BEA-MalDap₂ Fc)-NH₂ | |
| 363 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIe-αMeY-LIEGGGK[PEG₂₄-BEA-MalDap₂-Fc]-NH₂ | |
| 364 | Y-Aib-QGT-αMeF(2F)-TSD-4Pal-αMeS-I-αMeL-LD-Orn-K(ϵK2-γGlu-C18_OH)AQ-αMe4Pal-Orn- | 935 |
| | FIe-αMeY-LIEGSPSSGAPPPSK[PEG₂₄-BEA-MalDap₂-Fc]-NH₂ | |
| 365 | Y-Aib-QGT-αMeF(2F)-TSD-4Pal-αMeS-I-αMeL-LD-Orn-K(AEEA2-γGlu-C18_OH)AQ-αMe4Pal- | 935 |
| | Orn-FIQ-αMeY-LIEGSPSSGAPPPSK[PEG₂₄-BEA-MalDap₂-Fc]-NH₂ | |
| 366 | Y-Aib-QGT-αMeF(2F)-TSD-4Pal-αMeS-I-αMeL-LD-Orn-K(ϵK2-γGlu-C18_OH)AQ-αMe4Pal-Orn- | 935 |
| | FIQ-αMeY-LIEGSPSSGAPPPSK[PEG₂₄-BEA-MalDap₂-Fc]-NH₂ | |
| 367 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIQ-αMeY-LIEGSPSSGAPPPSK[PEG₂₄-BEA-MalDap₂-Fc]-NH₂ | |
| 368 | Y-Aib-QGT-αMeF(2F)-TSDY-αMeS-I-αMeL-LD-Orn-K(ϵK2-γGlu-C18_OH)AQ-αMe4Pal-Orn- | 935 |
| | FIQ-αMeY-LIEGSPSSGAPPPSK[PEG₂₄-BEA-MalDap₂-Fc]-NH₂ | |
| 369 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGSPSSGAPPPSK(AEEA6-GPAPAPAPAPAPA-BEA(MalDap₂-Fc)-NH₂ | |
| 370 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIE-Aib-SPSSGAPPPSK[PEG₂₄-BEA-MalDap₂-Fc]-NH₂ | |
| 371 | Y-Aib-QGT-αMeF(2F)-TSDY-αMeS-I-αMeL-LD-Orn-K(AEEA2-γGlu-C18_OH)AQ-αMe4Pal-EFIe- | 935 |
| | αMeY-LIEGSPSSGAPPPSK[PEG₂₄-BEA-MalDap₂-Fc]-NH₂ | |
| 372 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 373 | Y-Aib-QGT-αMeF(2F)-TSDY-αMeS-I-αMeL-LD-Orn-K(AEEA2-γGlu-C18_OH)AQ-Aib-EFIQ-αMeY- | 935 |
| | LIEGSPSSGAPPPSK[PEG₂₄-BEA-MalDap₂-Fc]-NH₂ | |
| 374 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LLEGSPSSGAPPPSK[PEG₂₄-BEA-MalDap₂-Fc]-NH₂ | |
| 375 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIEGGGGGGGSGGGGSGGGGSK[BEA-MalDap₂-Fc]-NH₂ | |
| 376 | Y-Aib-QGT-αMeF(2F)-TSD-4Pal-SI-αMeL-LD-Orn-K(AEEA2-γGlu-C18_OH)AQ-αMe4Pal-Orn- | 935 |
| | FIQ-αMeY-LIEGSPSSGAPPPSK[PEG₂₄-BEA-MalDap₂-Fc]-NH₂ | |
| 377 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIe-αMeY-LIE-Aib-GGGGGGSGGGGSGGGGSK[BEA-MalDap₂-Fc]-NH₂ | |
| 378 | Y-Aib-QaT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 379 | Y-αMeS-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 380 | Y-Iva-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 381 | Y-Aib-HGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 382 | H-Aib-HGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 383 | H-Aib-HGT-αMeF(2F)-TSDY-αMeS-I-αMeL-LD-Orn-K(AEEA2-γGlu-C18_OH)AQ-Aib-EFIQ-αMeY- | 935 |
| | LIEGSPSSGAPPPSK[PEG₂₄-BEA-MalDap₂-Fc]-NH₂ | |
| 384 | H-Aib-HGT-αMeF(2F)-TSD-4Pal-αMeS-I-αMeL-LD-Orn-K(AEEA2-γGlu-C18_OH)AQ-αMe4Pal- | 935 |
| | EFIQ-αMeY-LIEGSPSSGAPPPSK[PEG₂₄-BEA-MalDap₂-Fc]-NH₂ | |
| 385 | H-Aib-HGT-αMeF(2F)-TSD-4Pal-SI-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 386 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIE-Aib-SPSSGAPPPSK[PEG₂₄-BEA-MalDap₂-Fc]-NH₂ | |
| 387 | Y-Aib-QGT-αMeF(2F)-TSDY-αMeS-I-αMeL-LD-Orn-K(AEEA2-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 388 | Y-Aib-QGT-αMeF(2F)-TSD-4Pal-αMeS-I-αMeL-LD-Orn-K(AEEA2-γGlu-C18_OH)AQ-αMe4Pal- | 935 |
| | EFIQ-αMeY-LIEGSPSSGAPPPSK[PEG₂₄-BEA-MalDap₂-Fc]-NH₂ | |
| 389 | Y-Aib-QGT-αMeF(2F)-TSD-4Pal-αMeS-I-αMeL-LD-Orn-K(AEEA2-γGlu-C18_OH)AQ-αMe4Pal- | 935 |
| | EFIE-αMeY-LIEGSPSSGAPPPSK[PEG₂₄-BEA-MalDap₂-Fc]-NH₂ | |
| 390 | Y-Aib-QGT-αMeF(2F)-TSDY-αMeS-I-αMeL-LD-Orn-K(AEEA-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 391 | Y-Aib-QGT-αMeF(2F)-TSDY-αMeS-I-αMeL-LD-Orn-K(AEEA2-γGlu-C18_OH)AQ-αMe4Pal-EFIE- | 935 |
| | αMeY-LIE-Aib-SPSSGAPPPSK[PEG₂₄-BEA-MalDap₂-Fc]-NH₂ | |
| 392 | Y-Aib-QGT-αMeF(2F)-TSD-4Pal-αMeS-I-αMeL-LD-Orn-K(AEEA2-γGlu-C18_OH)AQ-αMe4Pal- | 935 |
| | EFIQ-αMeY-LIE-Aib-SPSSGAPPPSK[PEG₂₄-BEA-MalDap₂-Fc]-NH₂ | |
| 393 | Y-Aib-QGT-αMeF(2F)-TSD-4Pal-αMeS-I-αMeL-LD-Orn-K(AEEA2-γGlu-C18_OH)AQ-αMe4Pal- | 935 |
| | EFIE-αMeY-LIE-Aib-SPSSGAPPPSK[PEG₂₄-BEA-MalDap₂-Fc]-NH₂ | |
| 394 | Y-Aib-QGT-αMeF(2F)-TSDY-αMeS-I-αMeL-LD-Orn-K(AEEA-γGlu-C18_OH)AQ-αMe4Pal-EFIQ- | 935 |
| | αMeY-LIE-Aib-SPSSGAPPPSK[PEG₂₄-BEA-MalDap₂-Fc]-NH₂ | |
| 395 | Y-Aib-QGT-αMeF(2F)-TSDY-αMeS-I-αMeL-LDEK(AEEA-γGlu-C18_OH)AQ-αMe4Pal-EFIQ-αMeY- | 935 |
| | LLEGSPSSGAPPPSK[PEG₂₄-BEA-MalDap₂-Fc]-NH₂ | |
| 396 | Y-Aib-QGT-αMeF(2F)-TSDY-αMeS-I-αMeL-LDEK(AEEA-γGlu-C18_OH)AQ-αMe4Pal-EFIQ-αMeY- | 935 |
| | LLE-Aib-SPSSGAPPPSK[PEG₂₄-BEA-MalDap₂-Fc]-NH₂ | |
| 397 | H-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK[PEG₂₄-BEA-MalDap₂-Fc]-NH₂ | |
| 398 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LVEGSPSSGAPPPSK[PEG₂₄-BEA-MalDap₂-Fc]-NH₂ | |
| 399 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGGGGGGGSGGGGSGGGGSK[BEA-MalDap₂-Fc]-NH₂ | |
| 400 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIEGSPSSGAPPPSK(AEEA6-BEA-MalDap₂-Fc)-NH₂ | |
| 401 | H-Aib-HGT-αMeF(2F)-TSDY-αMeS-I-αMeL-LD-Orn-K(AEEA2-γGlu-C18_OH)AQ-Aib-EFIQ-αMeY- | 935 |
| | LLEGSPSSGAPPPSK[PEG₂₄-BEA-MalDap₂-Fc]-NH₂ | |
| 402 | H-Aib-HGT-αMeF(2F)-TSD-4Pal-SI-αMeL-LD-Orn-K(AEEA2-γGlu-C18_OH)AQ-αMe4Pal-EFIQ- | 935 |
| | αMeY-LLEGSPSSGAPPPSK[PEG₂₄-BEA-MalDap₂-Fc]-NH₂ | |
| 403 | Y-Aib-QGT-αMeF(2F)-TSDY-αMeS-I-αMeL-LD-Orn-K(AEEA-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 404 | Y-Aib-QGT-αMeF(2F)-TSD-4Pal-αMeS-I-αMeL-LD-Orn-K(AEEA-γGlu-C18_OH)AQ-αMe4Pal- | 935 |
| | EFIQ-αMeY-LIEGSPSSGAPPPSK[PEG₂₄-BEA-MalDap₂-Fc]-NH₂ | |
| 405 | Y-Aib-QGT-αMeF(2F)-TSDY-αMeS-I-αMeL-LD-Orn-K(AEEA2-γGlu-C18_OH)AQ-αMe4Pal-DFIQ- | 935 |
| | αMeY-LIEGSPSSGAPPPSK(AEEA6-BEA-MalDap₂-Fc)-NH₂ | |
| 406 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIE-αMeY-LIA-Aib-SPSSGAPPPSK(AEEA6-BEA-MalDap₂-Fc)-NH₂ | |
| 407 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LLEGSPSSGAPPPS-K(AEEA6-BEA-MalDap₂-Fc)-NH₂ | |
| 408 | Y-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LLE-Aib-SPSSGAPPPSK(AEEA6-BEA-MalDap₂-Fc)-NH₂ | |
| 409 | H-Aib-QGT-αMeF(2F)-TSDY-αMeS-I-αMeL-LD-Orn-K(AEEA2-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 410 | H-Aib-QGT-αMeF(2F)-TSDY-αMeS-I-αMeL-LD-Orn-K(AEEA2-γGlu-C18_OH)AQ-αMe4Pal-EFIE- | 935 |
| | αMeY-LIE-Aib-SPSSGAPPPSK[PEG₂₄-BEA-MalDap₂-Fc]-NH₂ | |
| 411 | H-Aib-QGT-αMeF(2F)-TSDY-αMeS-I-αMeL-LD-Orn-K(AEEA2-γGlu-C18_OH)AQ-αMe4Pal-EFIE- | 935 |
| | αMeY-LLEGSPSSGAPPPSK[PEG₂₄-BEA-MalDap₂-Fc]-NH₂ | |
| 412 | H-Aib-QGT-αMeF(2F)-TSDY-αMeS-I-αMeL-LD-Orn-K(AEEA2-γGlu-C18_OH)AQ-αMe4Pal-EFIE- | 935 |
| | αMeY-LLE-Aib-SPSSGAPPPSK[PEG₂₄-BEA-MalDap₂-Fc]-NH₂ | |
| 413 | H-Aib-QGT-αMeF(2F)-TSDY-αMeS-I-αMeL-LD-Orn-K(AEEA-γGlu-C18_OH)AQ-αMe4Pal-EFIE- | 935 |
| | αMeY-LIEGSPSSGAPPPSK[PEG₂₄-BEA-MalDap2-Fc]-NH₂ | |
| 414 | H-Aib-QGT-αMeF(2F)-TSDY-αMeS-I-αMeL-LD-Orn-K(AEEA-γGlu-C18_OH)AQ-αMe4Pal-EFIE- | 935 |
| | αMeY-LIE-Aib-SPSSGAPPPSK[PEG₂₄-BEA-MalDap₂-Fc]-NH₂ | |
| 415 | H-Aib-QGT-αMeF(2F)-TSDY-αMeS-I-αMeL-LD-Orn-K(AEEA-γGlu-C18_OH)AQ-αMe4Pal-EFIE- | 935 |
| | αMeY-LLEGSPSSGAPPPSK[PEG₂₄-BEA-MalDap₂-Fc]-NH₂ | |
| 416 | H-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LIE-Aib-SPSSGAPPPSK[PEG₂₄-BEA-MalDap₂-Fc]-NH₂ | |
| 417 | H-Aib-QGT-αMeF(2F)-TSDY-αMeS-I-αMeL-LD-Orn-K(AEEA2-γGlu-C18_OH)AQ-αMe4Pal-EFIQ- | 935 |
| | αMeY-LLE-Aib-SPSSGAPPPSK[PEG₂₄-BEA-MalDap₂-Fc]-NH₂ | |
| 418 | H-Aib-QGT-αMeF(2F)-TSDY-αMeS-I-αMeL-LD-Orn-K(AEEA-γGlu-C18_OH)AQ-αMe4Pal-EFIQ- | 935 |
| | αMeY-LIE-Aib-SPSSGAPPPSK[PEG₂₄-BEA-MalDap₂-Fc]-NH₂ | |
| 419 | H-Aib-QGT-αMeF(2F)-TSDY-αMeS-I-αMeL-LD-Orn-K(AEEA-γGlu-C18_OH)AQ-αMe4Pal-EFIQ- | 935 |
| | αMeY-LLE-Aib-SPSSGAPPPSK[PEG₂₄-BEA-MalDap₂-Fc]-NH₂ | |
| 420 | Y-αMeS-QGT-αMeF(2F)-TSDY-αMeS-I-αMeL-LD-Orn-K(AEEA-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 421 | Y-Iva-QGT-αMeF(2F)-TSDY-αMeS-I-αMeL-LD-Orn-K(AEEA-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 422 | Y-Ac4c-QGT-αMeF(2F)-TSDY-αMeS-I-αMeL-LD-Orn-K(AEEA-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 423 | H-Aib-QGT-αMeF(2F)-TSDY-αMeS-I-αMeL-LD-Orn-K(AEEA-γGlu-C18_OH)AQ-αMe4Pal-Orn- | 935 |
| | FIK[PEG₂₄-BEA-MalDap₂-Fc]-αMeY-LLEGSPSSGAPPPS-NH₂ | |
| 424 | H-Aib-QGT-αMeF(2F)-TSDY-αMeS-I-αMeL-LD-Orn-K(AEEA-γGlu-C18_OH)AQ-αMe4Pal- | 935 |
| | EFIK[PEG₂₄-BEA-MalDap₂-Fc]-αMeY-LLEGSPSSGAPPPS-NH₂ | |
| 425 | H-Aib-QGT-αMeF(2F)-TSDY-αMeS-I-αMeL-LD-Orn-K(AEEA-γGlu-C18_OH)AQ-αMe4Pal-Orn- | 935 |
| | FIe-αMeY-LIEGSPSSGAPPPSK[PEG₂₄-BEA-MalDap₂-Fc]-NH₂ | |
| 426 | H-Aib-QGT-αMeF(2F)-TSDY-αMeS-I-αMeL-LD-Orn-K(AEEA-γGlu-C18_OH)AQ-αMe4Pal-Orn- | 935 |
| | FIe-αMeY-LLEGSPSSGAPPPSK[PEG₂₄-BEA-MalDap₂-Fc]-NH₂ | |
| 427 | H-Aib-QGT-αMeF(2F)-TSDY-αMeS-I-αMeL-LD-Orn-K(AEEA-γGlu-C18_OH)AQ-αMe4Pal-Orn- | 935 |
| | FIE-αMeY-LIEGSPSSGAPPPSK[PEG₂₄-BEA-MalDap₂-Fc]-NH₂ | |
| 428 | H-Aib-QGT-αMeF(2F)-TSDY-αMeS-I-αMeL-LD-Orn-K(AEEA-γGlu-C18_OH)AQ-αMe4Pal-Orn- | 935 |
| | FIE-αMeY-LLEGSPSSGAPPPSK[PEG₂₄-BEA-MalDap₂-Fc]-NH₂ | |
| 429 | Y-Aib-QGT-αMeF(2F)-TSDY-αMeS-I-αMeL-LD-Orn-K(AEEA-γGlu-C18_OH)AQ-αMe4Pal-EFIE- | 935 |
| | αMeY-LLEGSPSSGAPPPSK[PEG₂₄-BEA-MalDap₂-Fc]-NH₂ | |
| 430 | Y-Aib-QGT-αMeF(2F)-TSDY-αMeS-I-αMeL-LD-Orn-K(AEEA-γGlu-C18_OH)AQ-αMe4Pal-EFIe- | 935 |
| | αMeY-LLEGSPSSGAPPPSK[PEG₂₄-BEA-MalDap₂-Fc]-NH₂ | |
| 431 | Y-Aib-QGT-αMeF(2F)-TSDY-αMeS-I-αMeL-LD-Orn-K(AEEA-γGlu-C18_OH)AQ-αMe4Pal-Orn- | 935 |
| | FIE-αMeY-LLEGSPSSGAPPPSK[PEG₂₄-BEA-MalDap₂-Fc]-NH₂ | |
| 432 | Y-Aib-QGT-αMeF(2F)-TSDY-αMeS-I-αMeL-LD-Orn-K(AEEA-γGlu-C18_OH)AQ-αMe4Pal-Orn- | 935 |
| | FIe-αMeY-LLEGSPSSGAPPPSK[PEG₂₄-BEA-MalDap₂-Fc]-NH₂ | |
| 433 | Y-Aib-QGT-αMeF(2F)-TSDY-αMeS-I-αMeL-LD-Orn-K(AEEA-γGlu-C18_OH)AQ-αMe4Pal-EFIe- | 935 |
| | αMeY-LIEGSPSSGAPPPSK[PEG₂₄-BEA-MalDap₂-Fc]-NH₂ | |
| 434 | Y-Aib-QGT-αMeF(2F)-TSDY-αMeS-I-αMeL-LD-Orn-K(AEEA-γGlu-C18_OH)AQ-αMe4Pal-Orn- | 935 |
| | FIE-αMeY-LIEGSPSSGAPPPSK[PEG₂₄-BEA-MalDap₂-Fc]-NH₂ | |
| 435 | Y-Aib-QGT-αMeF(2F)-TSDY-αMeS-I-αMeL-LD-Orn-K(AEEA-γGlu-C18_OH)AQ-αMe4Pal-Orn- | 935 |
| | FIe-αMeY-LIEGSPSSGAPPPSK[PEG₂₄-BEA-MalDap₂-Fc]-NH₂ | |
| 436 | Y-Aib-QGT-αMeF(2F)-TSDY-αMeS-I-αMeL-LD-Orn-K(AEEA-γGlu-C18_OH)AQ-Aib-QFIE-αMeY- | 935 |
| | LLEGSPSSGAPPPSK[PEG₂₄-BEA-MalDap₂-Fc]-NH₂ | |
| 437 | Y-Aib-QGT-αMeF(2F)-TSDY-αMeS-I-αMeL-LD-Orn-K(AEEA-γGlu-C18_OH)AQ-Aib-Orn-FIe- | 935 |
| | αMeY-LLEGSPSSGAPPPSK[PEG₂₄-BEA-MalDap₂-Fc]-NH₂ | |
| 438 | Y-Aib-QGT-αMeF(2F)-TSDY-αMeS-I-αMeL-LD-Orn-K(AEEA-γGlu-C18_OH)AQ-Aib-Orn-FIE- | 935 |
| | αMeY-LLEGSPSSGAPPPSK[PEG₂₄-BEA-MalDap₂-Fc]-NH₂ | |
| 439 | Y-Aib-QGT-αMeF(2F)-TSDY-αMeS-I-αMeL-LD-Orn-K(AEEA-γGlu-C18_OH)AQ-Aib-Orn-FIq- | 935 |
| | αMeY-LLEGSPSSGAPPPSK[PEG₂₄-BEA-MalDap₂-Fc]-NH₂ | |
| 440 | Y-Aib-QGT-αMeF(2F)-TSDY-αMeS-I-αMeL-LD-Orn-K(AEEA-γGlu-C18_OH)AQ-Aib-Orn-FIQ- | 935 |
| | αMeY-LLEGSPSSGAPPPSK[PEG₂₄-BEA-MalDap₂-Fc]-NH₂ | |
| 441 | Y-Aib-QGT-αMeF(2F)-TSDY-αMeS-I-αMeL-LD-Orn-K(AEEA-γGlu-C18_OH)AQ-Aib-EFIe-αMeY- | 935 |
| | LLEGSPSSGAPPPSK[PEG₂₄-BEA-MalDap₂-Fc]-NH₂ | |
| 442 | Y-Aib-QGT-αMeF(2F)-TSDY-αMeS-I-αMeL-LD-Orn-K(AEEA-γGlu-C18_OH)AQ-Aib-EFIE-αMeY- | 935 |
| | LLEGSPSSGAPPPSK[PEG₂₄-BEA-MalDap₂-Fc]-NH₂ | |
| 443 | Y-Aib-QGT-αMeF(2F)-TSDY-αMeS-I-αMeL-LD-Orn-K(AEEA-γGlu-C18_OH)AQ-Aib-EFIq-αMeY- | 935 |
| | LLEGSPSSGAPPPSK[PEG₂₄-BEA-MalDap₂-Fc]-NH₂ | |
| 444 | H-Ac4c-QGT-αMeF(2F)-TSDY-αMeS-I-αMeL-LD-Orn-K(AEEA-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 445 | YaQGT-αMeF(2F)-TSDY-αMeS-I-αMeL-LD-Orn-K(AEEA-γGlu-C18_OH)AQ-αMe4Pal-EFIE-αMeY- | 935 |
| | LIEGSPSSGAPPPSK[PEG₂₄-BEA-MalDap₂-Fc]-NH₂ | |
| 446 | YaQGT-αMeF(2F)-TSDY-αMeS-I-αMeL-LD-Orn-K(AEEA-γGlu-C18_OH)AQ-αMe4Pal-Orn-FIE- | 935 |
| | αMeY-LIEGSPSSGAPPPSK[PEG₂₄-BEA-MalDap₂-Fc]-NH₂ | |
| 447 | YaQGT-αMeF(2F)-TSDY-αMeS-I-αMeL-LD-Orn-K(AEEA-γGlu-C18_OH)AQ-αMe4Pal-Orn-FIe- | 935 |
| | αMeY-LIEGSPSSGAPPPSK[PEG₂₄-BEA-MalDap₂-Fc]-NH₂ | |
| 448 | YaQGT-αMeF(2F)-TSDY-αMeS-I-αMeL-LD-Orn-K(AEEA-γGlu-C18_OH)AQ-αMe4Pal-EFIE-αMeY- | 935 |
| | LLEGSPSSGAPPPSK[PEG₂₄-BEA-MalDap₂-Fc]-NH₂ | |
| 449 | YaQGT-αMeF(2F)-TSDY-αMeS-I-αMeL-LD-Orn-K(AEEA-γGlu-C18_OH)AQ-αMe4Pal-EFIe-αMeY- | 935 |
| | LLEGSPSSGAPPPSK[PEG₂₄-BEA-MalDap₂-Fc]-NH₂ | |
| 450 | YaQGT-αMeF(2F)-TSDY-αMeS-I-αMeL-LD-Orn-K(AEEA-γGlu-C18_OH)AQ-αMe4Pal-Orn-FIE- | 935 |
| | αMeY-LLEGSPSSGAPPPSK[PEG₂₄-BEA-MalDap₂-Fc]-NH₂ | |
| 451 | YaQGT-αMeF(2F)-TSDY-αMeS-I-αMeL-LD-Orn-K(AEEA-γGlu-C18_OH)AQ-Aib-Orn-FIq-αMeY- | 935 |
| | LLEGSPSSGAPPPSK[PEG₂₄-BEA-MalDap₂-Fc]-NH₂ | |
| 452 | YaQGT-αMeF(2F)-TSDY-αMeS-I-αMeL-LD-Orn-K(AEEA-γGlu-C18_OH)AQ-Aib-Orn-FIQ-αMeY- | 935 |
| | LLEGSPSSGAPPPSK[PEG₂₄-BEA-MalDap₂-Fc]-NH₂ | |
| 453 | YaQGT-αMeF(2F)-TSDY-αMeS-I-αMeL-LD-Orn-K(AEEA-γGlu-C18_OH)AQ-Aib-Orn-FIE-αMeY- | 935 |
| | LIEGSPSSGAPPPSK[PEG₂₄-BEA-MalDap₂-Fc]-NH₂ | |
| 454 | Y-Aib-QGT-αMeF(2F)-TSD-4Pal-SI-αMeL-LD-Orn-K(AEEA-γGlu-C18_OH)AQ-αMe4Pal-Orn- | 935 |
| | FIE-αMeY-LIEGSPSSGAPPPSK[PEG₂₄-BEA-MalDap₂-Fc]-NH₂ | |
| 455 | Y-Aib-QGT-αMeF(2F)-TSD-4Pal-αMeS-I-αMeL-LD-Orn-K(AEEA-γGlu-C18_OH)AQ-αMe4Pal- | 935 |
| | Orn-FIe-αMeY-LIEGSPSSGAPPPSK[PEG₂₄-BEA-MalDap₂-Fc]-NH₂ | |
| 456 | Y-Aib-QGT-αMeF(2F)-TSD-4Pal-αMeS-I-αMeL-LD-Orn-K(AEEA-γGlu-C18_OH)AQ-αMe4Pal- | 935 |
| | EFIE-αMeY-LIEGSPSSGAPPPSK[PEG₂₄-BEA-MalDap₂-Fc]-NH₂ | |
| 457 | Y-Aib-QGT-αMeF(2F)-TSD-4Pal-SI-αMeL-LD-Orn-K(AEEA-γGlu-C18_OH)AQ-αMe4Pal-EFIE- | 935 |
| | αMeY-LIEGSPSSGAPPPSK[PEG₂₄-BEA-MalDap₂-Fc]-NH₂ | |
| 458 | Y-Aib-QGT-αMeF(2F)-TSDY-αMeS-I-αMeL-LD-Orn-K(AEEA-γGlu-C18_OH)AQ-Aib-Orn-FIE- | 935 |
| | αMeY-LIEGSPSSGAPPPSK[PEG₂₄-BEA-MalDap₂-Fc]-NH₂ | |
| 459 | Y-Aib-QGT-αMeF(2F)-TSD-4Pal-αMeS-I-αMeL-LD-Orn-K(AEEA-γGlu-C18_OH)AQ-αMe4Pal- | 935 |
| | EFIe-αMeY-LIEGSPSSGAPPPSK[PEG₂₄-BEA-MalDap₂-Fc]-NH₂ | |
| 460 | YaQGT-αMeF(2F)-TSDY-αMeS-I-αMeL-LD-Orn-K(AEEA2-γGlu-C18_OH)AQ-αMe4Pal-Orn-FIE- | 935 |
| | αMeY-LIEGSPSSGAPPPSK[PEG₂₄-BEA-MalDap₂-Fc]-NH₂ | |
| 461 | YaQGT-αMeF(2F)-TSDY-αMeS-I-αMeL-LD-Orn-K(AEEA2-γGlu-C18_OH)AQ-αMe4Pal-Orn-FIE- | 935 |
| | αMeY-LLEGSPSSGAPPPSK[PEG₂₄-BEA-MalDap₂-Fc]-NH₂ | |
| 462 | Y-Aib-QGT-αMeF(2F)-TSDY-αMeS-I-αMeL-LD-Orn-K(AEEA-γGlu-C18_OH)AQ-Aib-Orn-FIe- | 935 |
| | αMeY-LIEGSPSSGAPPPSK[PEG₂₄-BEA-MalDap₂-Fc]-NH₂ | |
| 463 | NMeY-Aib-QGT-αMeF(2F)-TSDY-αMeS-I-αMeL-LD-Orn-K(AEEA2-γGlu-C18_OH)AQ-αMe4Pal- | 935 |
| | EFIE-αMeY-LIEGSPSSGAPPPSK[PEG₂₄-BEA-MalDap₂-Fc]-NH₂ | |
| 464 | Y-Aib-QGT-αMeF(2F)-TSDY-αMeS-I-αMeL-LD-Orn-K(AEEA2-γGlu-C20_OH)AQ-αMe4Pal-Orn- | 935 |
| | FIe-αMeY-LIEGSPSSGAPPPSK[PEG₂₄-BEA-MalDap₂-Fc]-NH₂ | |
| 465 | Y-Aib-QGT-αMeF(2F)-TSDY-αMeS-I-αMeL-LD-Orn-K(AEEA2-γGlu-C20_OH)AQ-αMe4Pal-Orn- | 935 |
| | FIQ-αMeY-LIEGSPSSGAPPPSK[PEG₂₄-BEA-MalDap₂-Fc]-NH₂ | |
| 466 | Y-Aib-QGT-αMeF(2F)-TSD-4Pal-αMeS-I-αMeL-LD-Orn-K(AEEA2-γGlu-C18_OH)AQ-αMe4Pal- | 936 |
| | Orn-FIe-αMeY-LIEGSPSSGAPPPSK(PEG₂₄-BEA-MalDap₂-Fc KRQ)-NH₂ | |
| 467 | Y-Aib-QGT-αMeF(2F)-TSDY-αMeS-I-αMeL-LD-Orn-K(AEEA2-γGlu-C18_OH)AQ-αMe4Pal-Orn- | 936 |
| | FIe-αMeY-LIEGSPSSGAPPPSK[PEG₂₄-BEA-MalDap₂-Fc KRQ]-NH₂ | |
| 468 | Y-Aib-QGT-αMeF(2F)-TSD-4Pal-αMeS-I-αMeL-LD-Orn-K(AEEA2-γGlu-C18_OH)AQ-αMe4Pal- | 936 |
| | Orn-FIE-αMeY-LIEGGGGGGGSGGGGSGAPAPAPAPAPAPK[BEA-MalDap₂-Fc KRQ]-NH₂ | |
| 469 | Y-Aib-QGT-αMeF(2F)-TSDY-αMeS-I-αMeL-LD-Orn-K(AEEA2-γGlu-C18_OH)AQ-αMe4Pal-Orn- | 936 |
| | FIe-αMeY-LIEGGGGGGGSGGGGSGAPAPAPAPAPAPK[BEA-MalDap₂-Fc KRQ]-NH₂ | |
| 470 | Y-Aib-QGT-αMeF(2F)-TSDY-αMeS-I-αMeL-LD-Orn-K(AEEA2-γGlu-C18 OH)AQ-αMe4Pal-Orn- | 936 |
| | FIe-αMeY-LIEGSPSSGAPPPSK(AEEA6-GPAPAPAPAPAPA-[BEAMalDap₂-Fc KRQ]-NH₂ | |
| 471 | Y-Aib-QGT-αMeF(2F)-TSDY-αMeS-I-αMeL-LD-Orn-K(AEEA2-γGlu-C18_OH)AQ-αMe4Pal-Orn- | 936 |
| | FIe-αMeY-LIEGSPSSGAPPPSK[PEG₂₄-BEA-MalDap₂-Fc KRQ]-NH₂ | |
| 472 | Y-Aib-QGT-αMeF(2F)-TSDY-αMeS-I-αMeL-LD-Orn-K(AEEA2-γGlu-C18_OH)AQ-αMe4Pal-Orn- | 936 |
| | FIe-αMeY-LIE-Aib-SPSSGAPPPSK[PEG₂₄-BEA-MalDap₂-Fc KRQ]-NH₂ | |
| 473 | Y-Aib-QGT-αMeF(2F)-TSDY-αMeS-I-αMeL-LD-Orn-K(AEEA2-γGlu-C18_OH)AQ-αMe4Pal-Orn- | 936 |
| | FIe-αMeY-LIEGSPSSGAPPPSK(AEEA6-BEA-MalDap₂-Fc KRQ)-NH₂ | |
| 474 | Y-Aib-QGT-αMeF(2F)-TSDY-αMeS-I-αMeL-LD-Orn-K(AEEA2-γGlu-C18_OH)AQ-αMe4Pal-Orn- | 941 |
| | FIe-αMeY-LIE-Aib-GGGGGGSGGGGSGAPAPAPAPAPAPK[BEA-MalDap₂-Fc IG KRQ]-NH₂ | |
| 475 | Y-Aib-QGT-αMeF(2F)-TSDY-αMeS-I-αMeL-LD-Orn-K(AEEA2-γGlu-C18_OH)AQ-αMe4Pal-Orn- | 941 |
| | FIe-αMeY-LIE-Aib-SPSSGAPPPSK(AEEA6-GPAPAPAPAPAPA-[BEA-MalDap₂-Fc IG KRQ]-NH₂ | |
| 476 | Y-Aib-QGT-αMeF(2F)-TSDY-αMeS-I-αMeL-LD-Orn-K(AEEA2-γGlu-C18_OH)AQ-αMe4Pal-Orn- | 941 |
| | FIe-αMeY-LIE-Aib-SPSSGAPPPSK[AEEA6-BEA-MalDap₂-Fc IG KRQ]-NH₂ | |
| 477 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIE-αMeY-LIE-Aib-SPSSGAPPPSK[PEG₂₄-BEA-MalDap₂-Fc]-NH₂ | |
| 478 | Y-Aib-QGT-αMeF(2F)-TSDY-αMeS-I-αMeL-LD-Orn-K(AEEA2-γGlu-C18_OH)AQ-αMe4Pal-Orn- | 935 |
| | FIQ-αMeY-LIE-Aib-SPSSGAPPPSK[PEG₂₄-BEA-MalDap₂-Fc]-NH₂ | |
| 479 | Y-Aib-EGT-αMeF(2F)-TSD-4Pal-SI-αMeL-LDEK(AEEA2-γGlu-C18_OH)AQ-Aib-EFIe-αMeY- | 935 |
| | LIEGSPSSGAPPPSK[PEG₂₄-dCAP]-Fc-NH₂ | |
| 480 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPSK[PEG₂₄-BEA-(MSPT)₂-Fc]-NH₂ | |
| 481 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPSK[PEG₂₄-BEA-(Acetyl)₂-Fc]-NH₂ | |
| 482 | Y-Aib-EGT-αMeF(2F)-TSD-4Pal-SI-αMeL-LD-Orn-K(ϵK2-γGlu-C18_OH)AQ-Aib-EFIe-αMeY- | 935 |
| | LIEGSPSSGAPPPSK[PEG₂₄-BEA-(OD)₂-Fc]-NH₂ | |

Embodiment 22. A compound comprising any of the following Fc-acylated polypeptides:

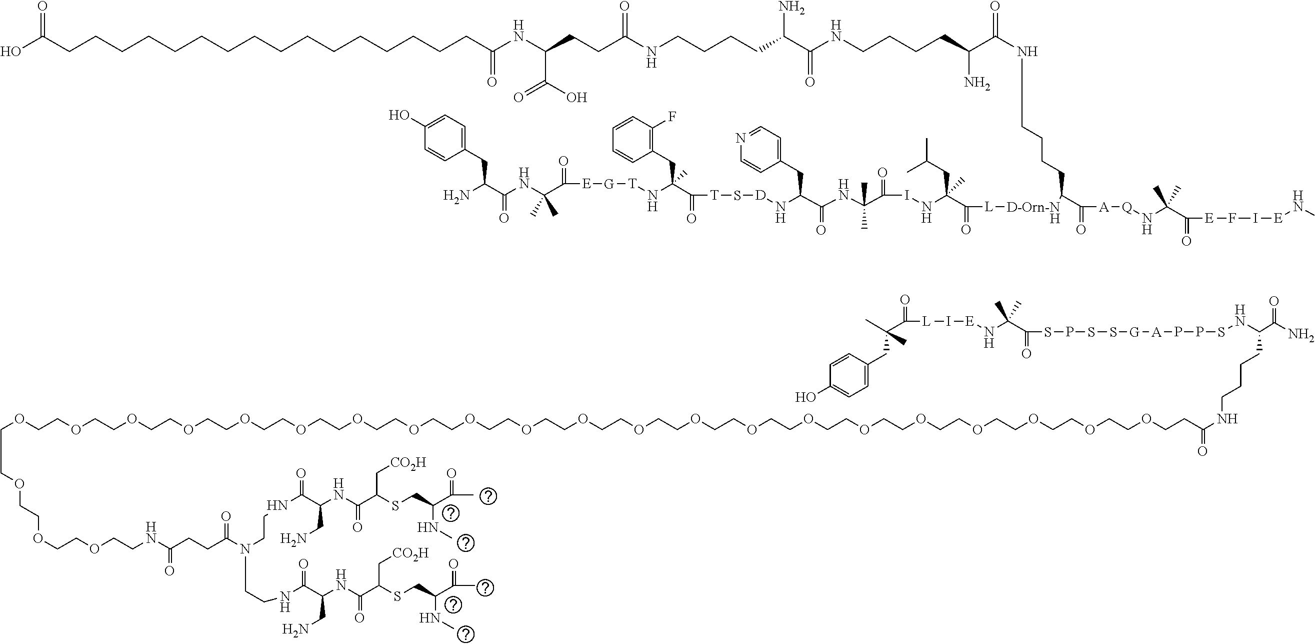

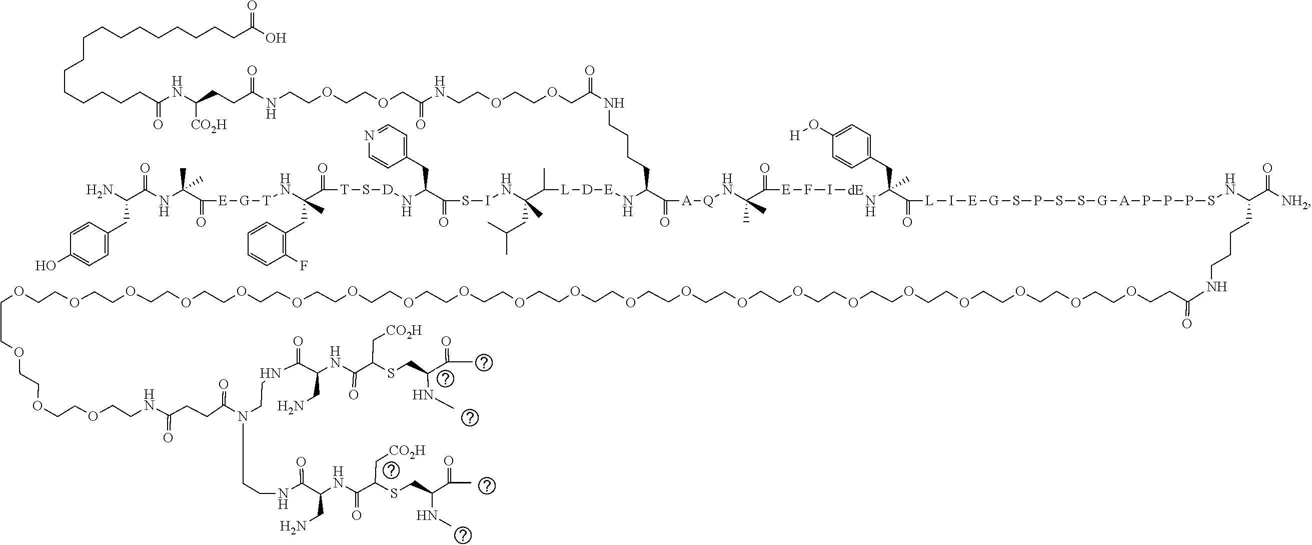

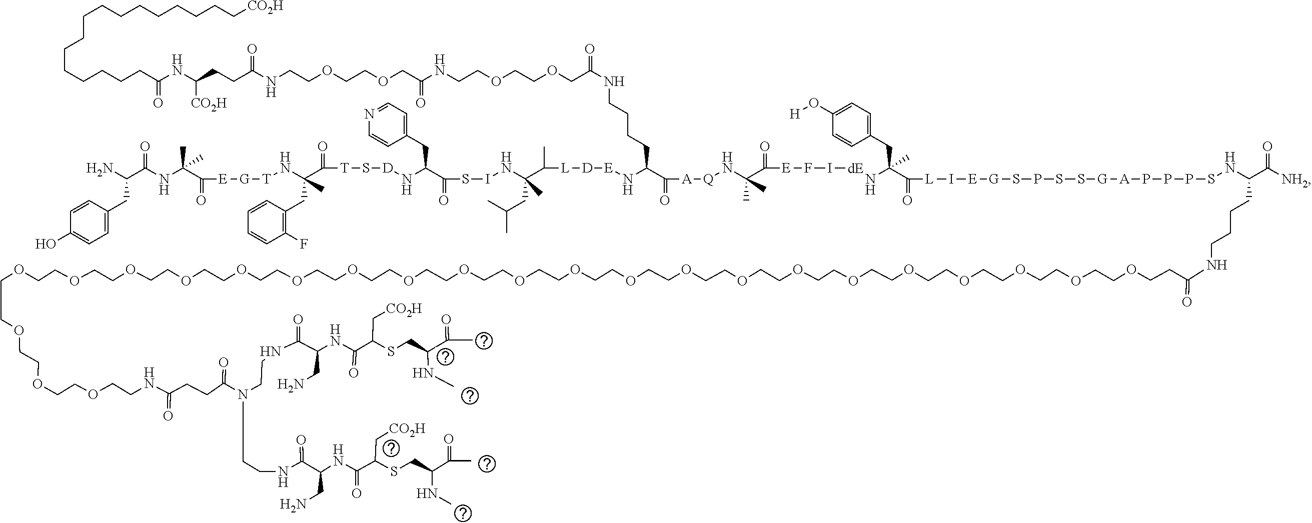

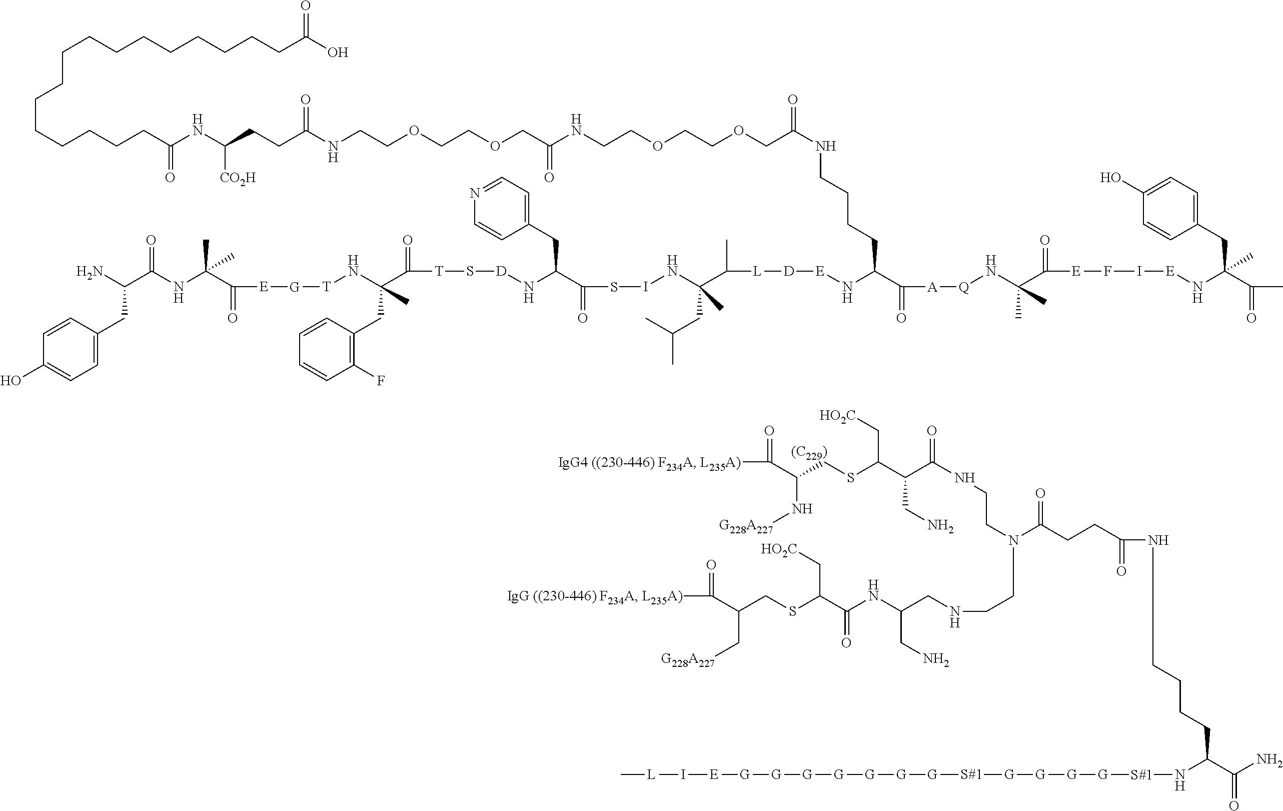

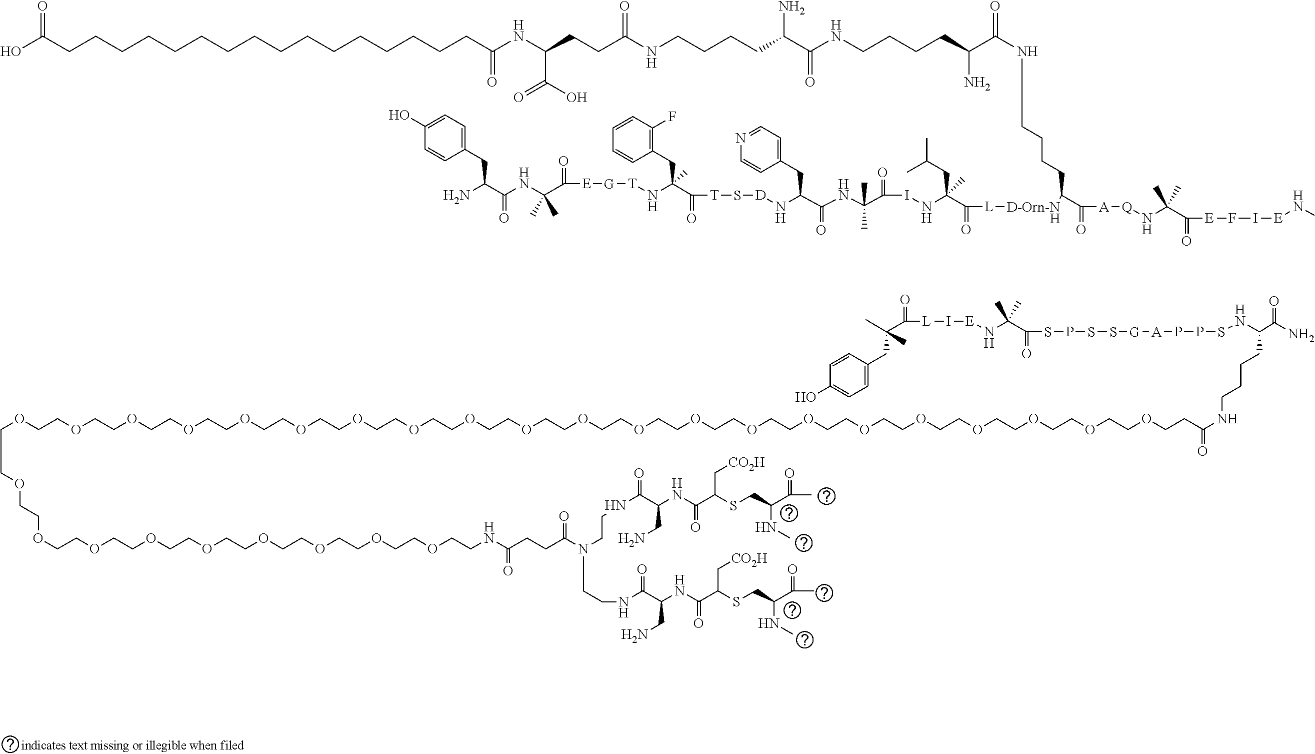

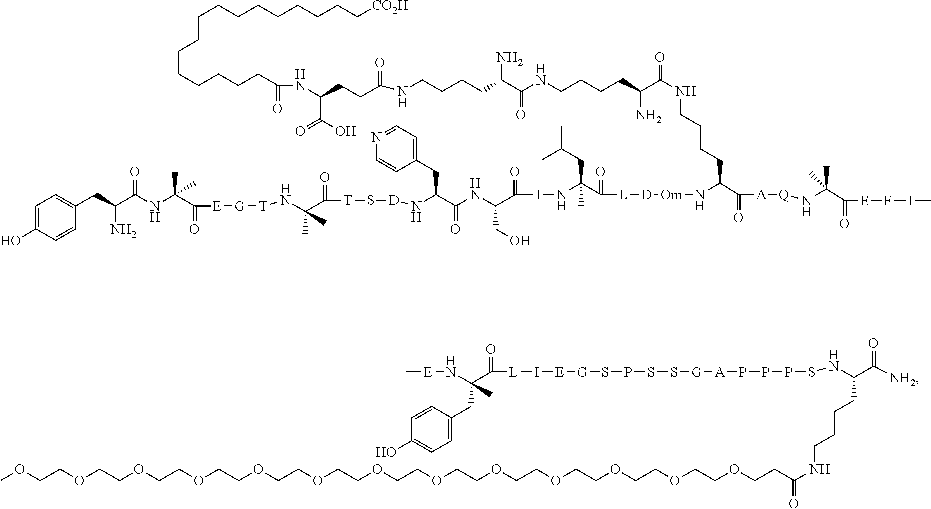

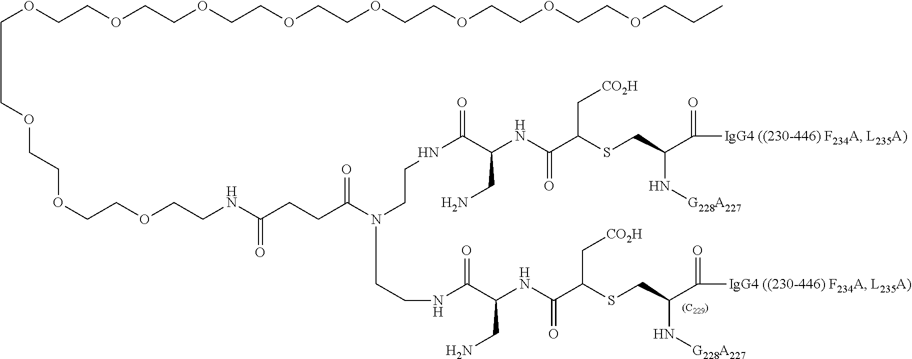

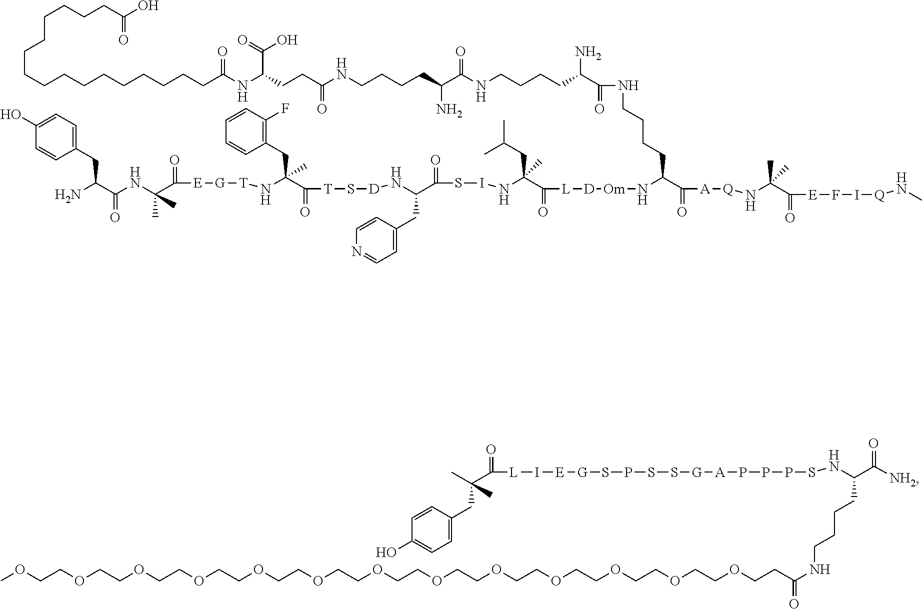

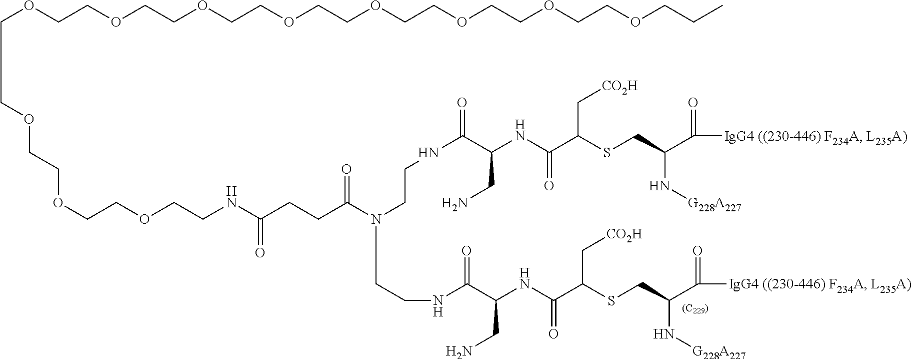

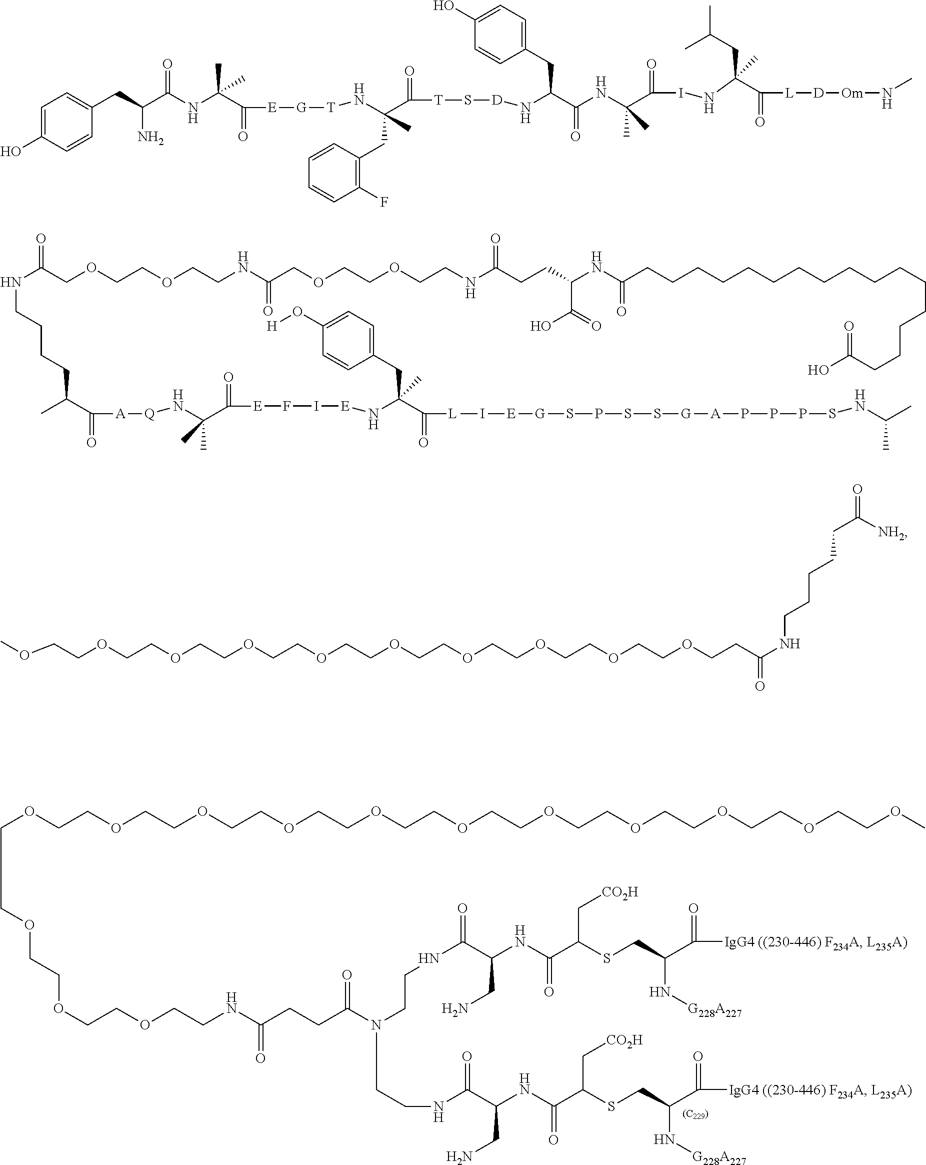

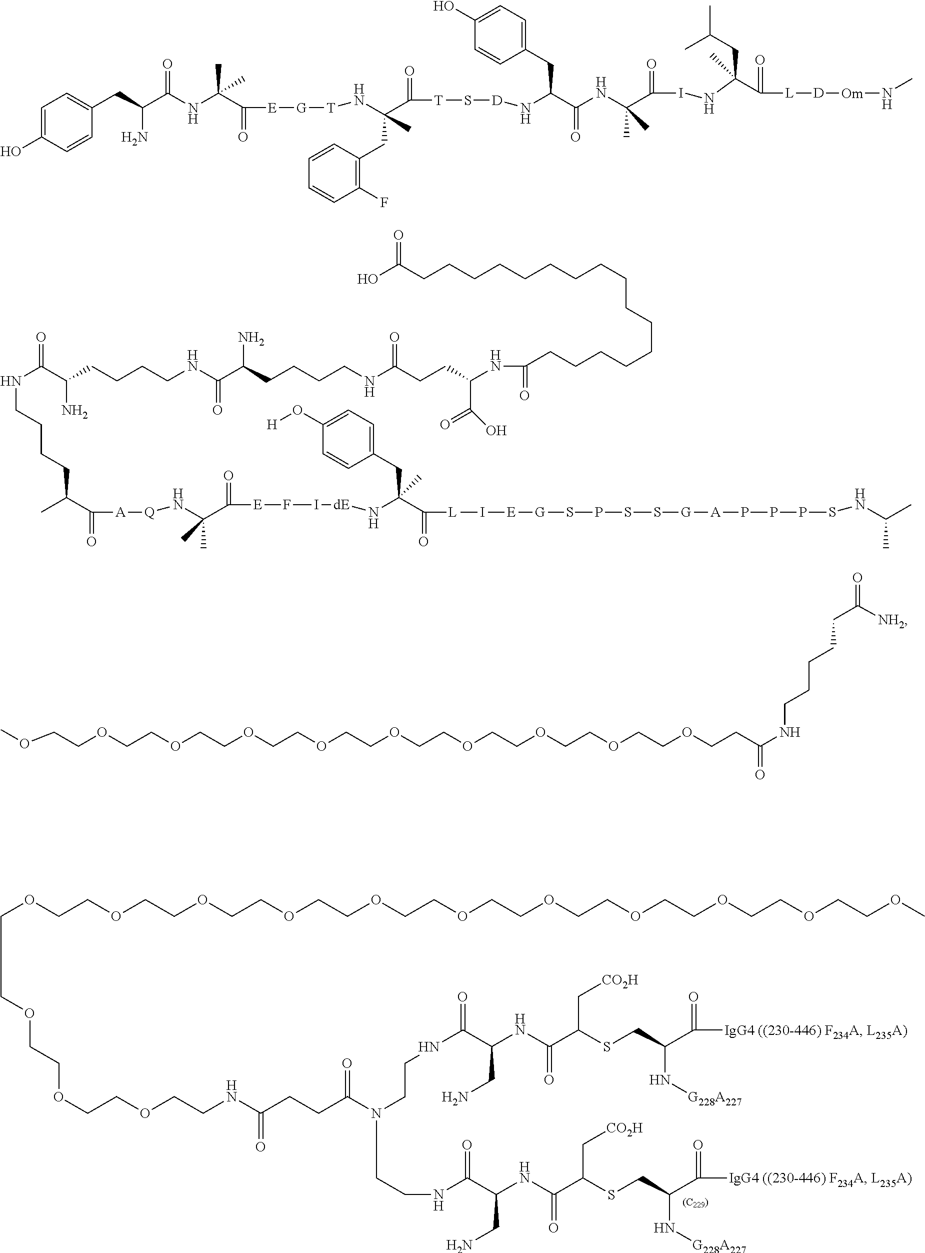

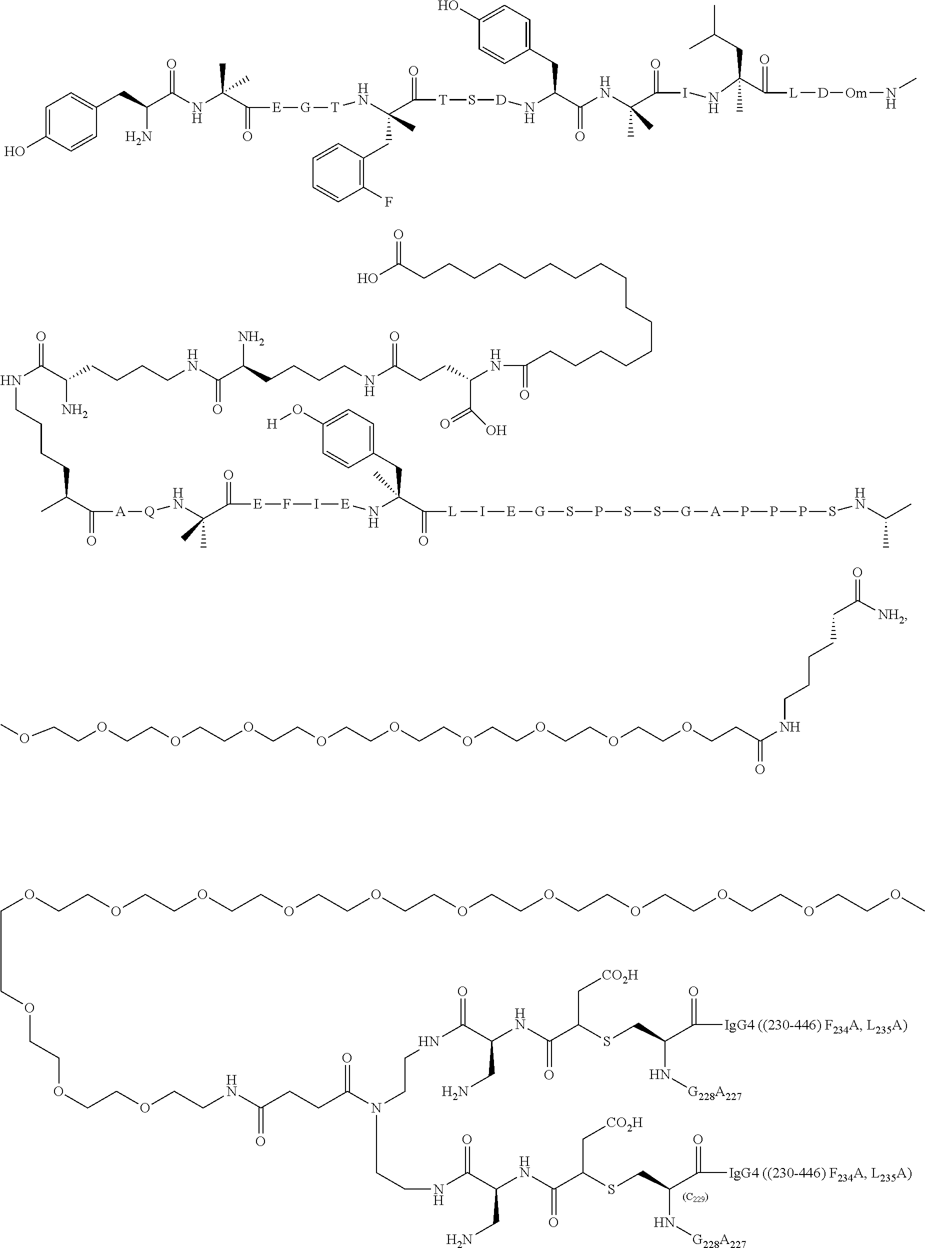

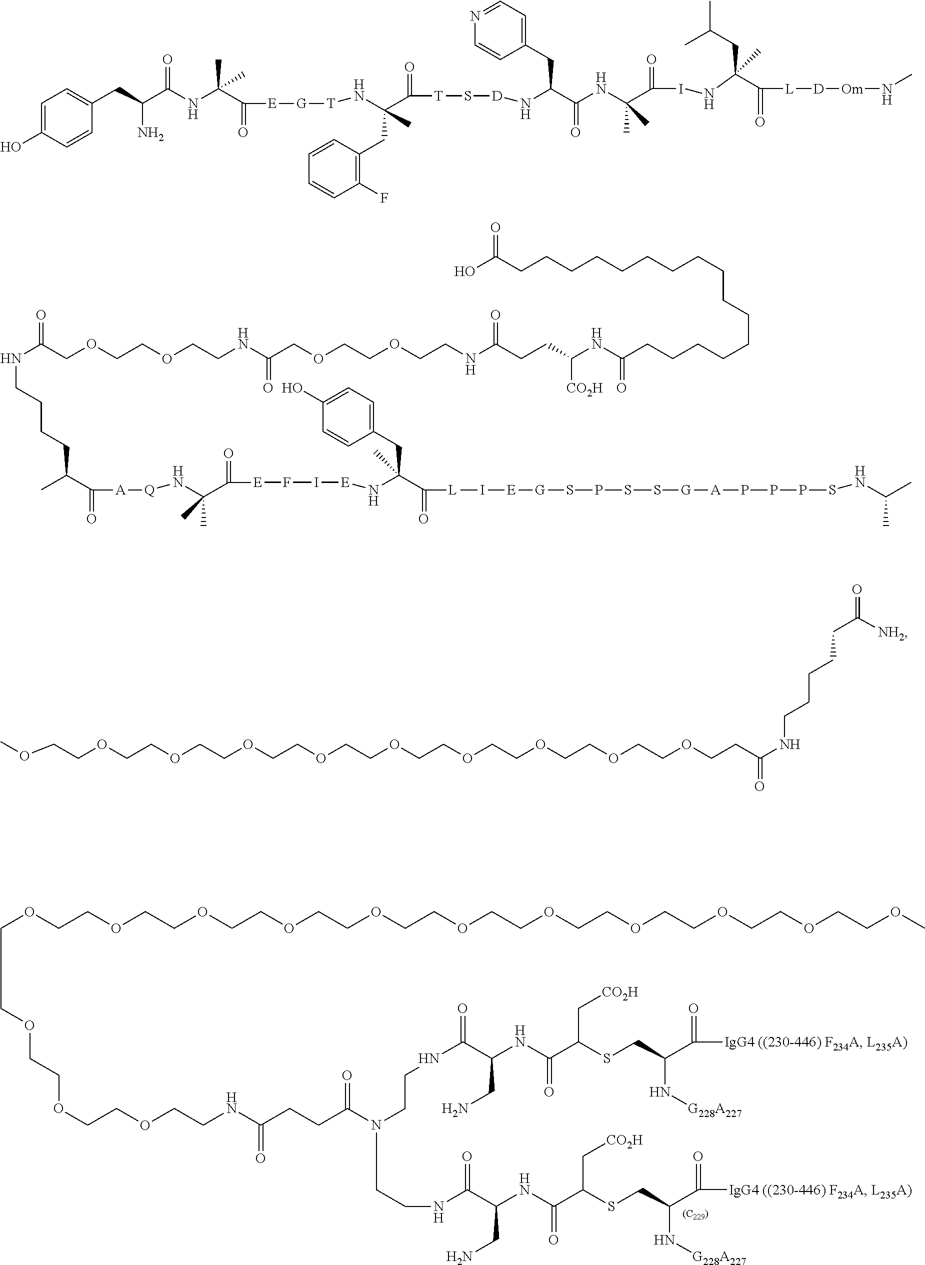

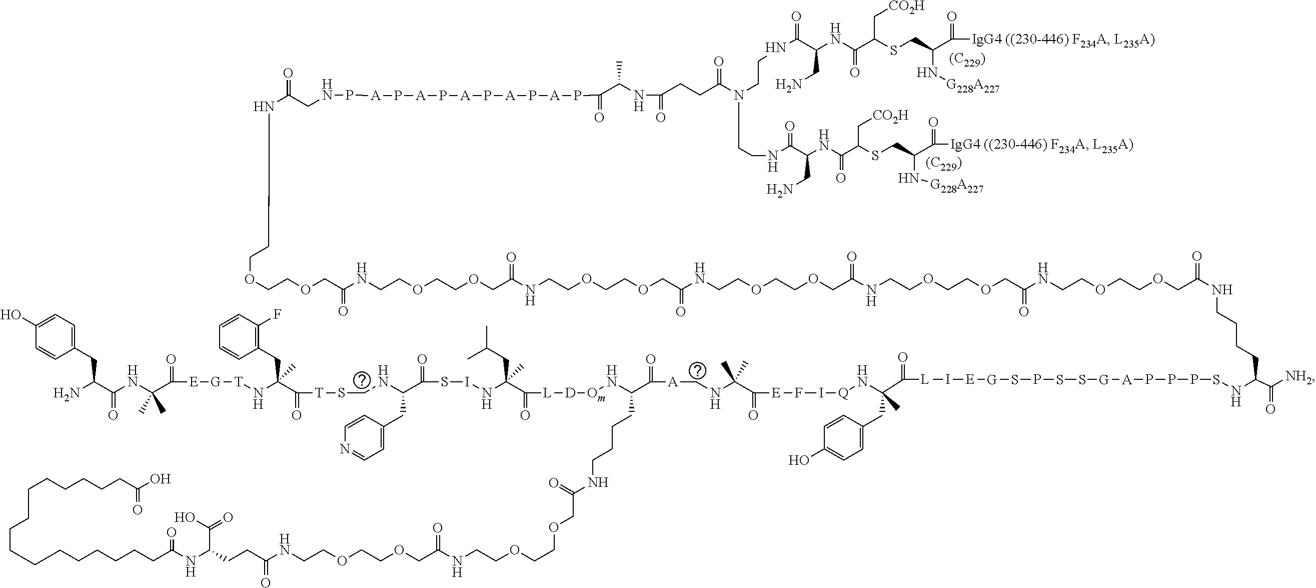

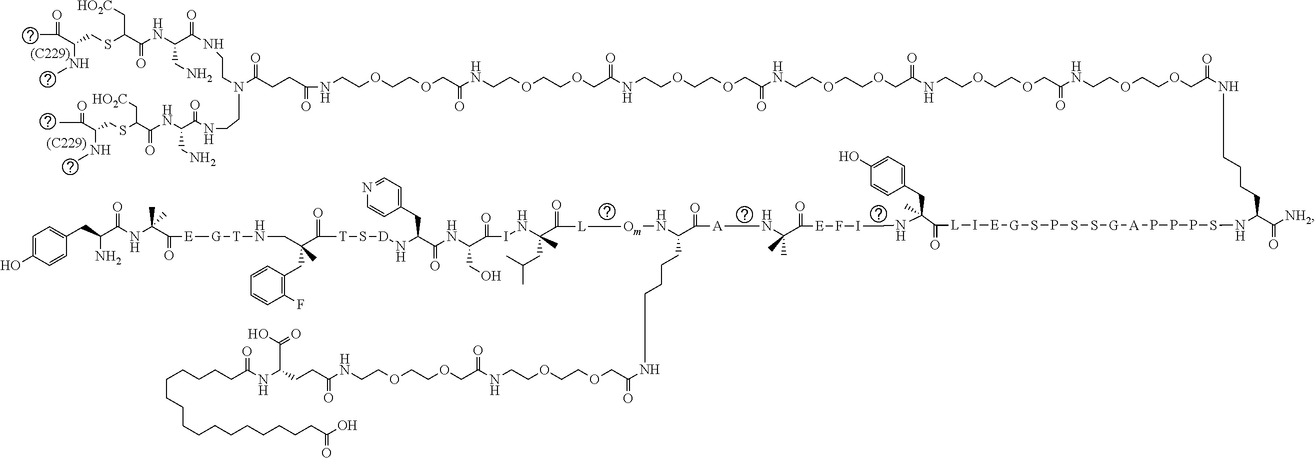

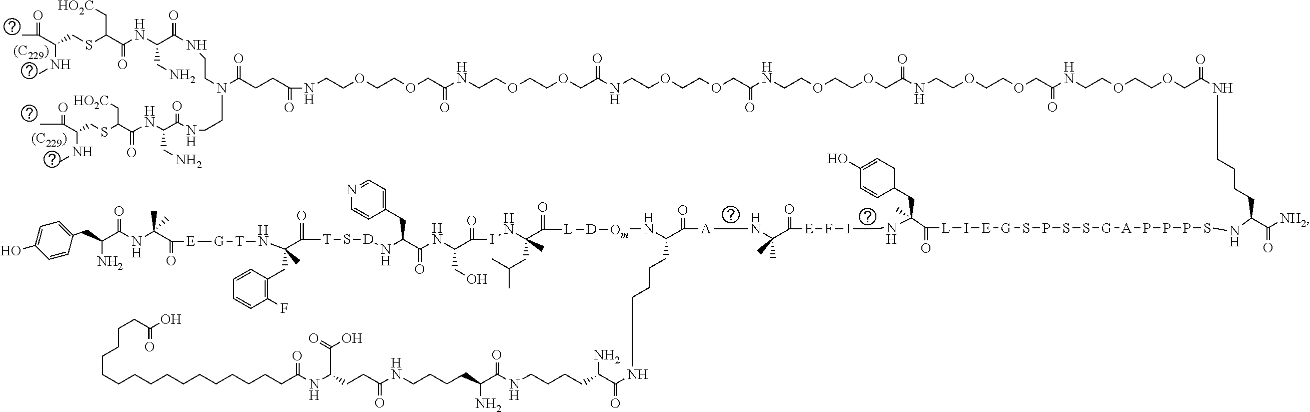

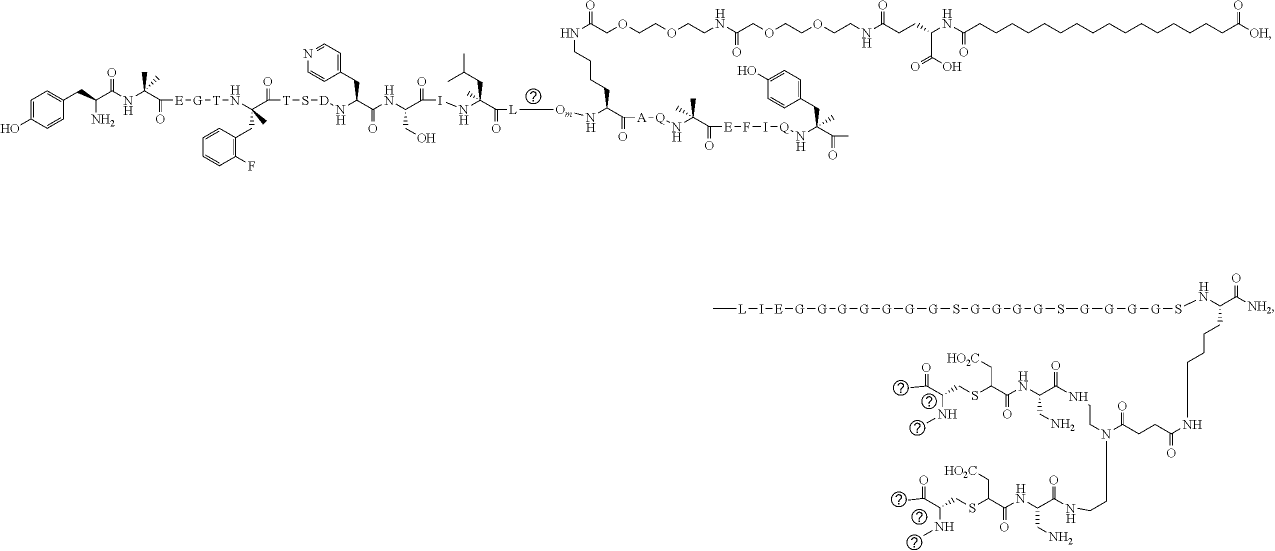

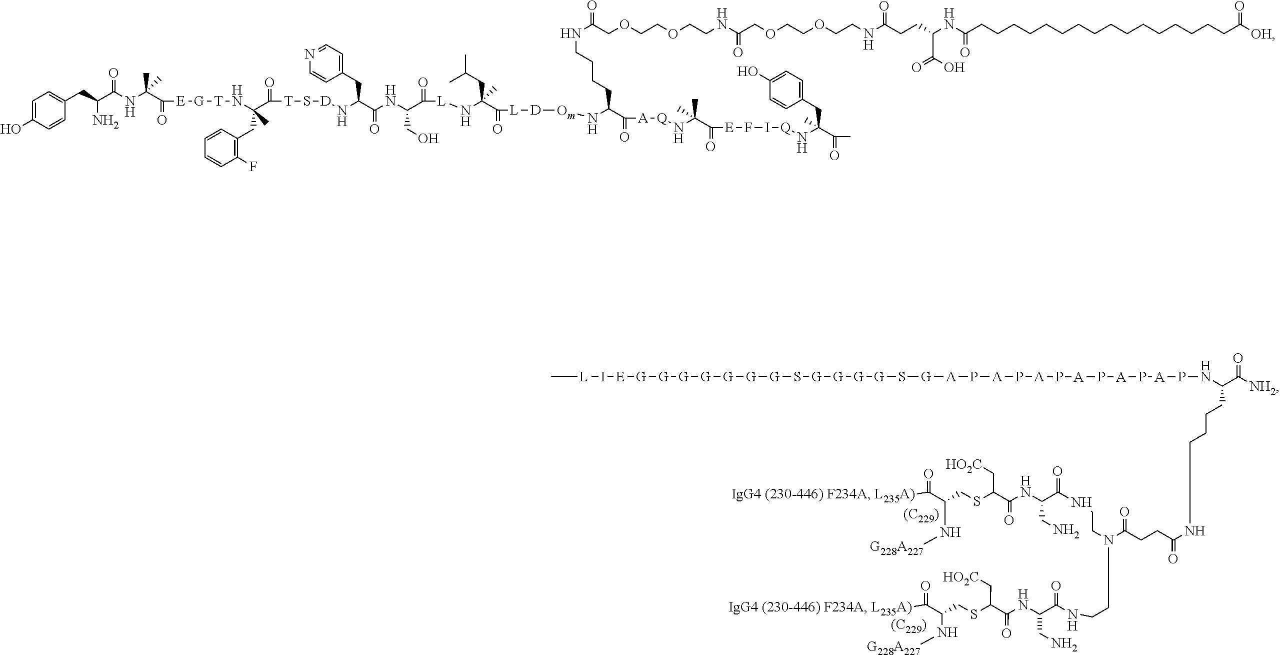

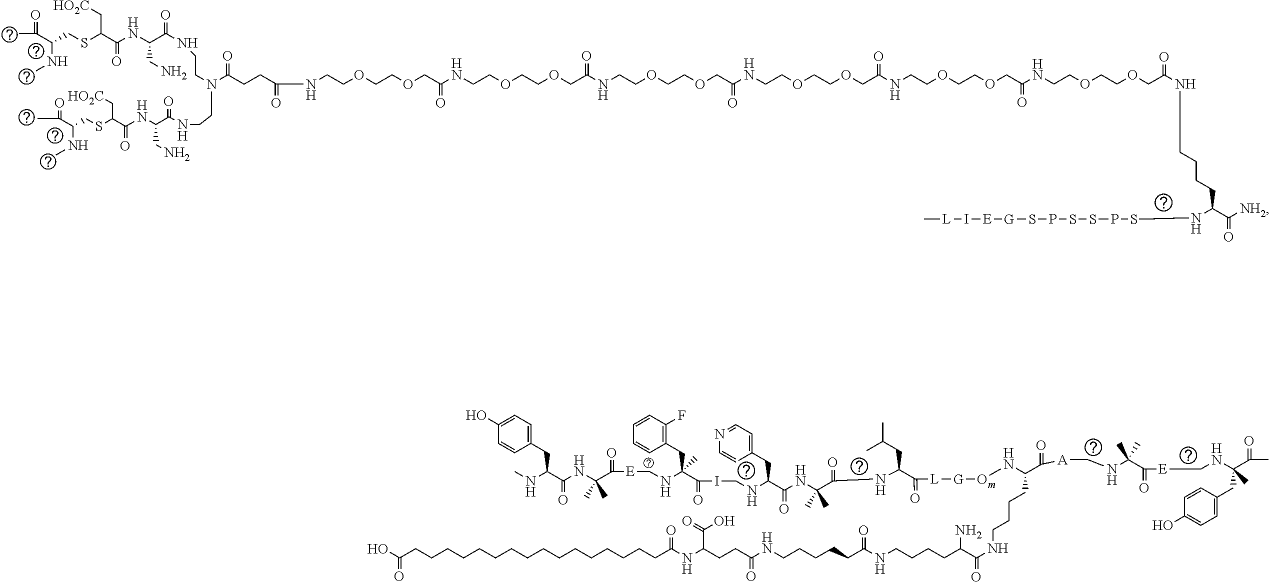

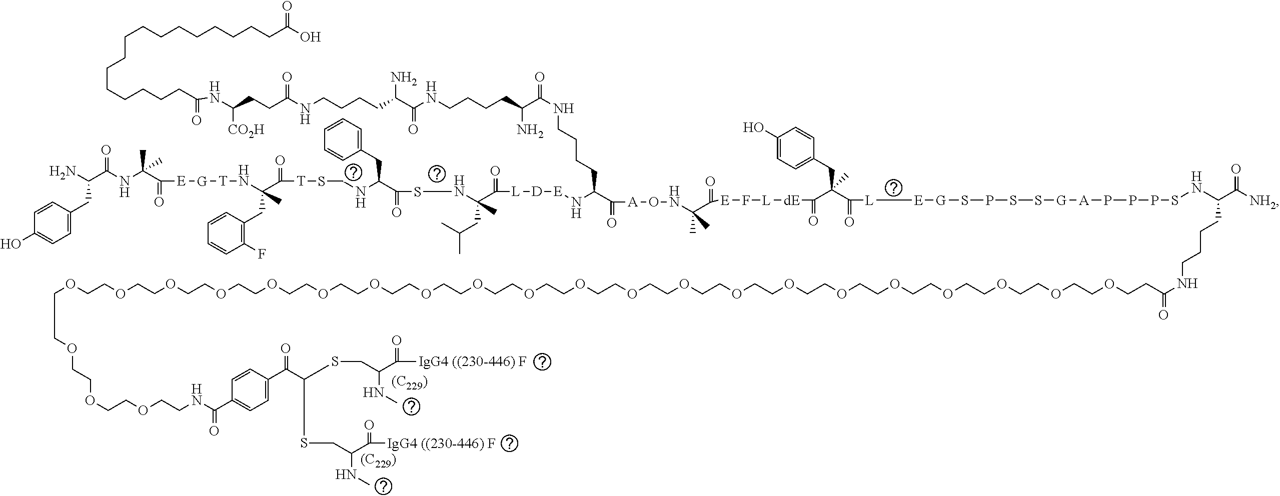

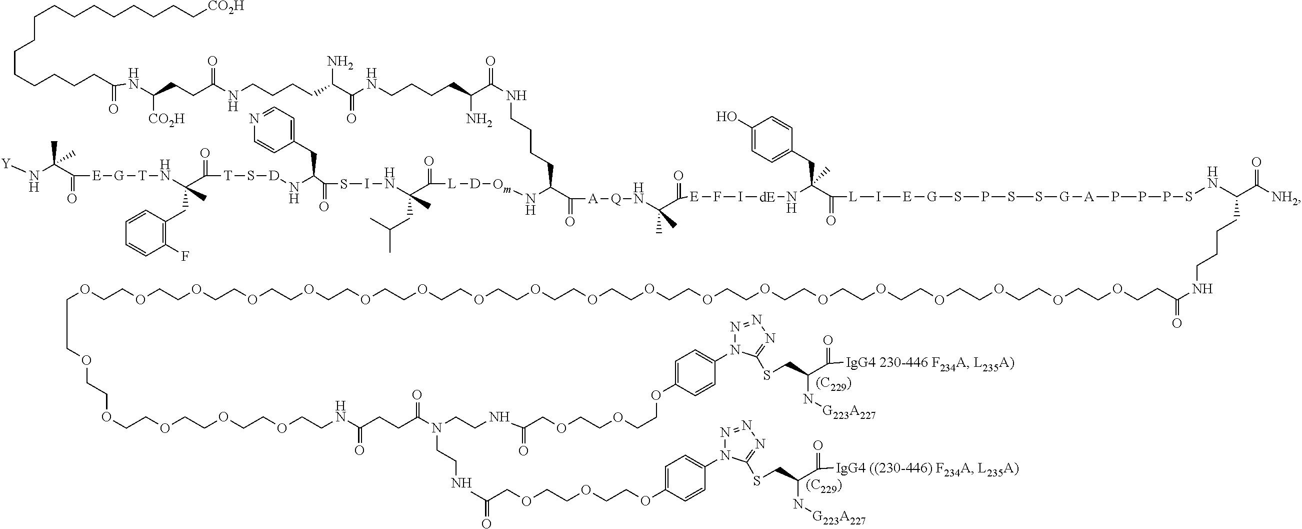

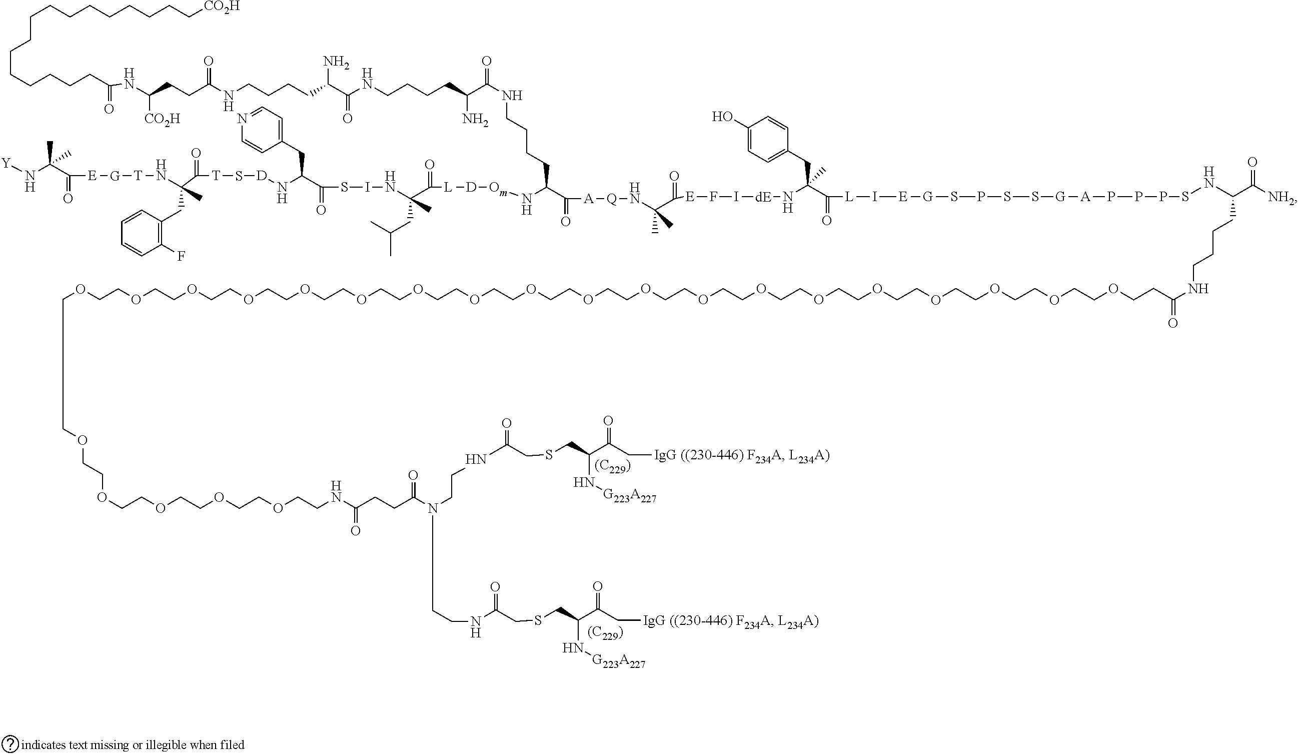

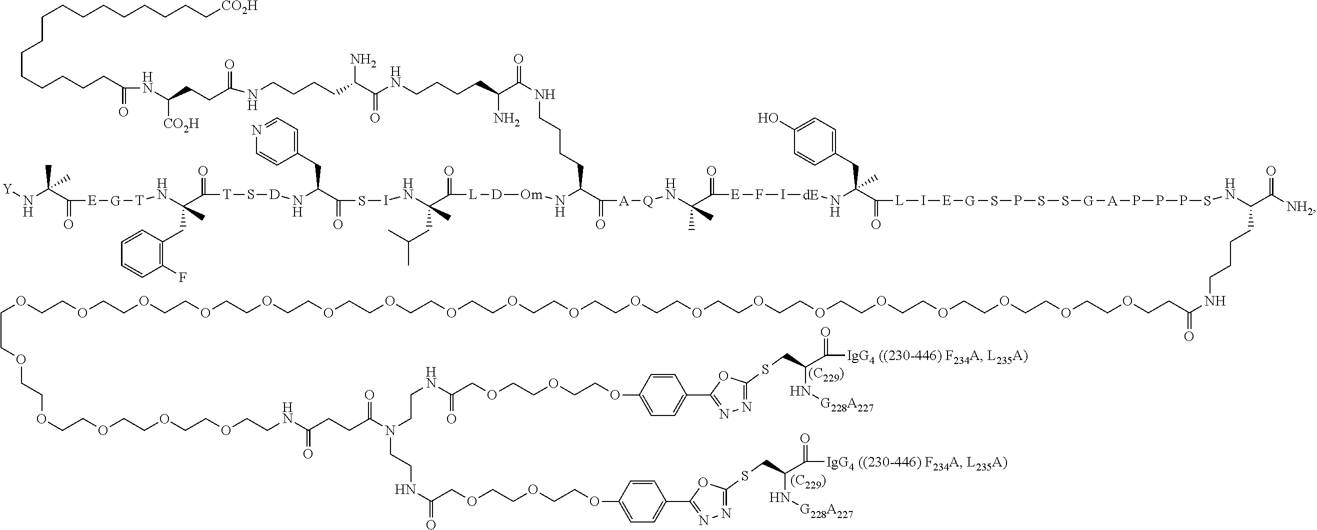

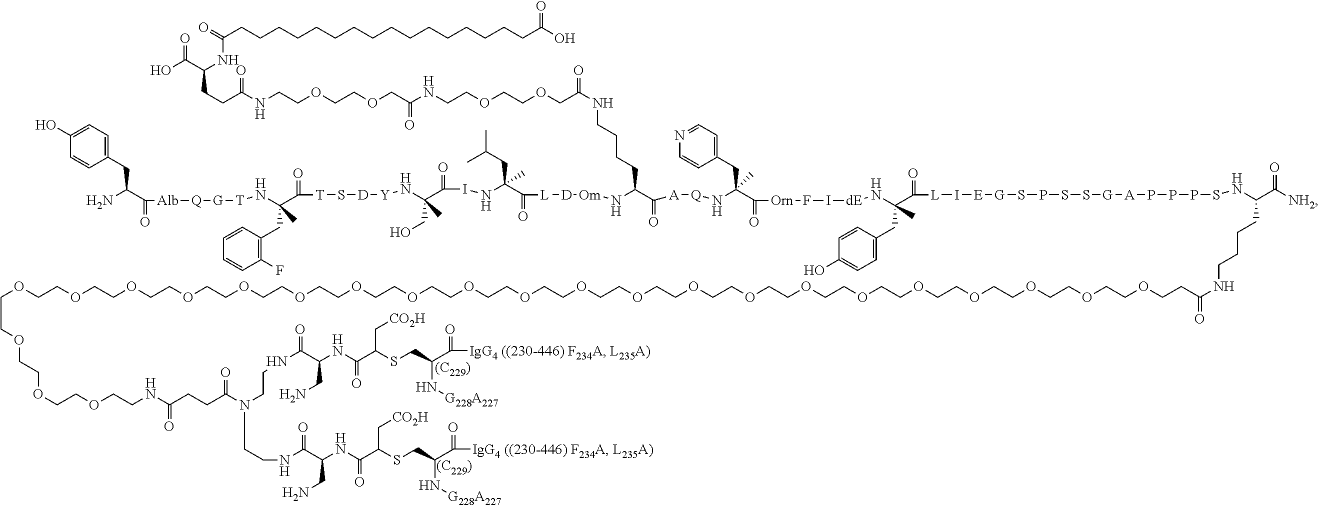

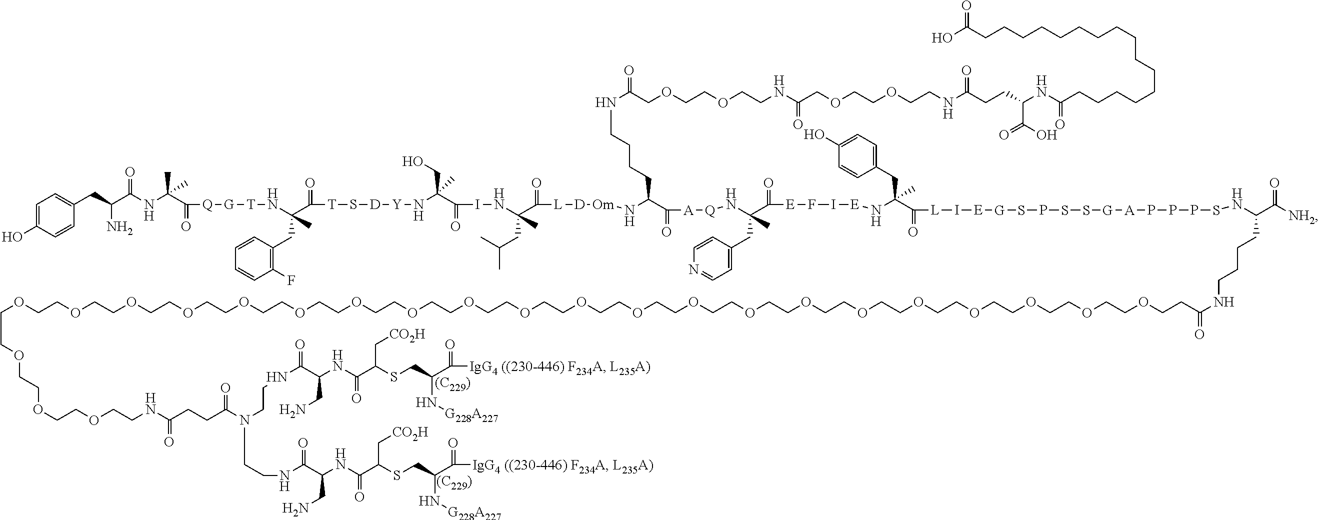

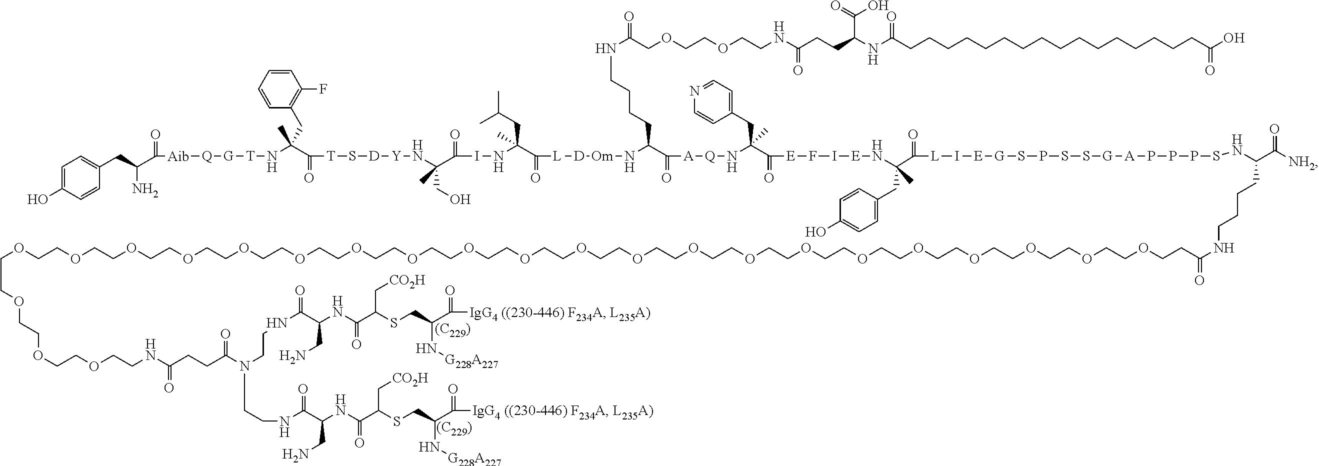

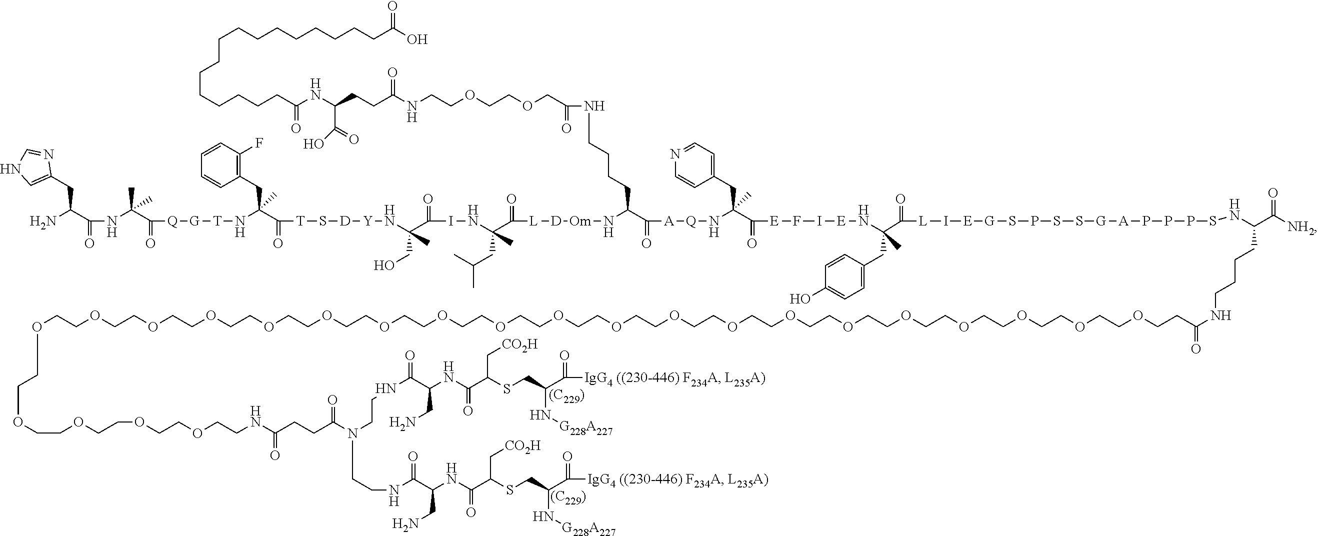

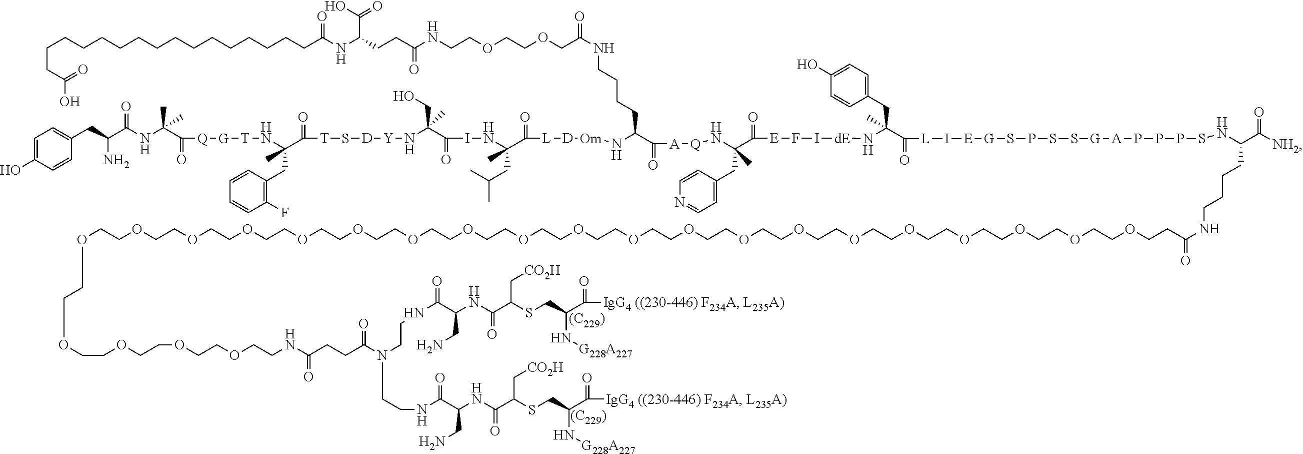

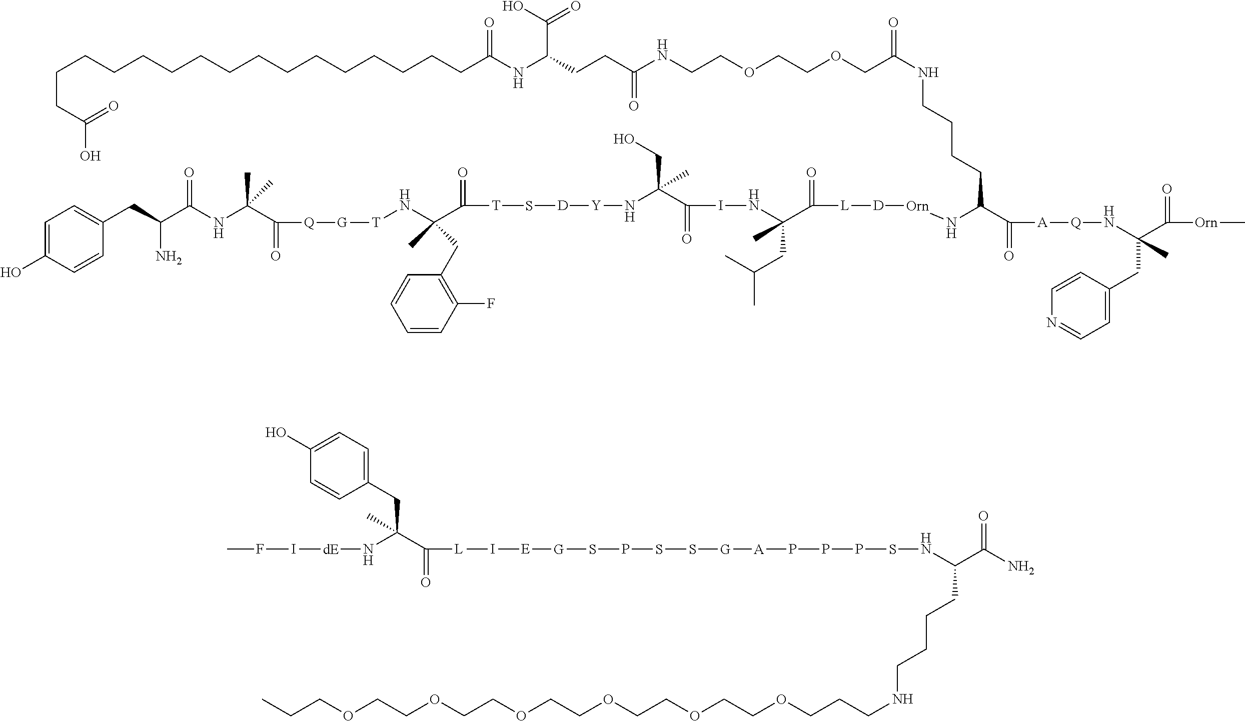

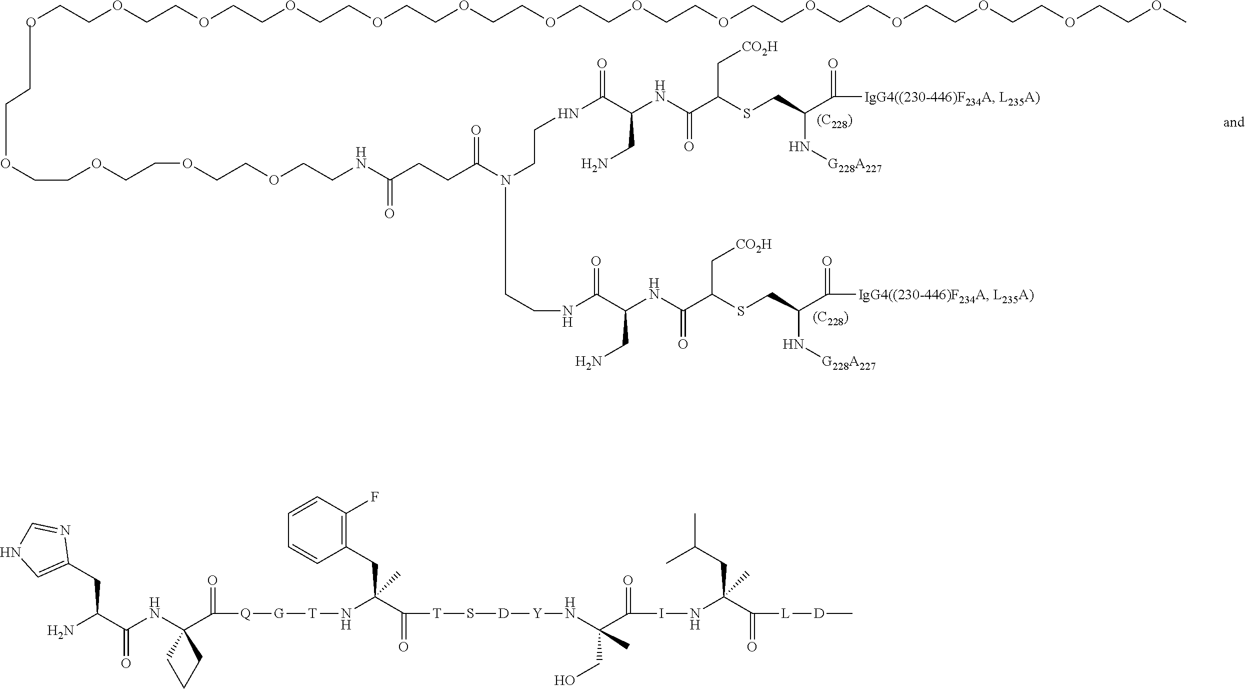

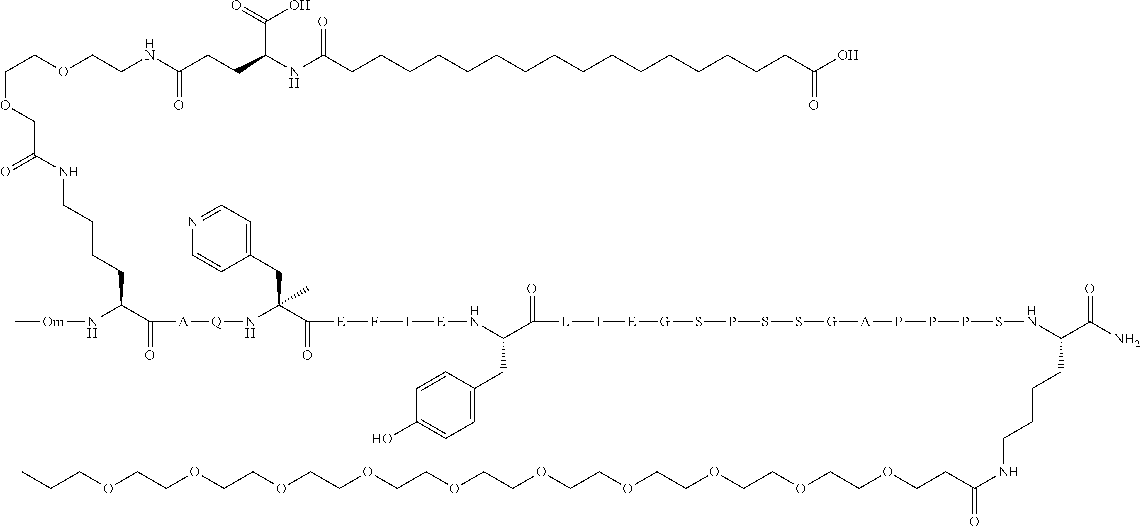

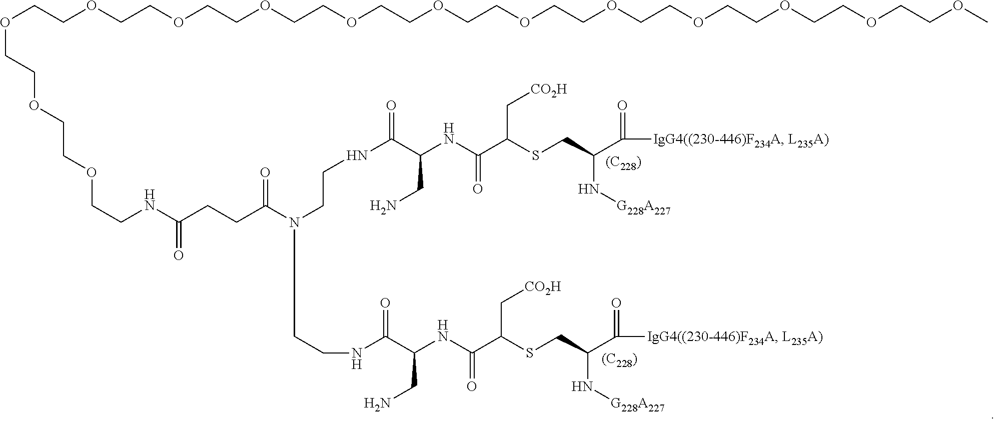

Embodiment 22. The compound of any one of embodiments 1-22, or a pharmaceutically acceptable salt thereof, wherein the compound has greater potency at each of the GIP and GLP-1 receptors as compared to native GIP (SEQ ID NO:1223) and GLP-17-36 (SEQ ID NO:1221).

Embodiment 23. The compound of any one of embodiments 1-22, or a pharmaceutically acceptable salt thereof, wherein the compound has greater potency at each of the glucagon, GIP and GLP-1 receptors as compared to native glucagon (SEQ ID NO:1222), GIP (SEQ ID NO:1223) and GLP-1₇₋₃₆ (SEQ ID NO:1221).

Embodiment 24. The compound of any one of embodiments 1-23, or a pharmaceutically acceptable salt thereof, wherein the compound has a sufficiently extended duration of action to allow for dosing as infrequently as once-monthly.

Embodiment 25. A pharmaceutical composition comprising the compound of any one of embodiments 1-24, or a pharmaceutically acceptable salt thereof, and at least one pharmaceutically acceptable carrier, diluent, or excipient.

Embodiment 26. The pharmaceutical composition of embodiment 25, wherein the composition is formulated for subcutaneous administration.

Embodiment 27. A method of treating a disease or condition selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA) polycystic ovary syndrome (PCOS), Parkinson's disease and Alzheimer's disease, the method comprising a step of: administering to an individual in need thereof an effective amount of a compound, or a pharmaceutically acceptable salt thereof, of any one of embodiments 1 to 26.

The invention is further illustrated by the following examples, which are not to be construed as limiting.

### EXAMPLES

### Preparation of Therapeutic Polypeptides

Processes for making exemplary compounds of the present disclosure are described below. It will be understood that these processes are not exclusive, and that compounds of the present disclosure may also be prepared using other processes known to persons skilled in the art.

Peptide intermediates intended for Fc conjugation are fully synthesized following conventional fluorenylmethyloxycarbonyl (Fmoc)/tert-Butyl (t-Bu) solid-phase peptide synthesis (SPPS) protocols. This process is carried out using the Symphony 12-Channel Multiplex Peptide Synthesizer (Protein Technologies, Inc., Tucson, AZ).

A linear peptide backbone with orthogonally protected lysine residues (4-methyltrityl (Mtt) and ivDde) at designated sequence positions is first synthesized. Following this, sequential deprotection and coupling steps introduce the ‘Linker-Fatty Acid’ moiety, followed by the ‘Linker-Fc Conjugation’ moiety. These modifications complete the synthesis of peptide intermediates before they are cleaved from the solid support and undergo subsequent purification, as described below:

### Procedure for Peptide Synthesis

Solid-phase peptide synthesis (SPPS) is conducted using 1% DVB cross-linked polystyrene resin (Fmoc-Rink-MBHA, 100-200 mesh, Chem-Impex International) with a substitution range of 0.3-0.6 mmol/g. Peptide backbones are assembled with N-Fmoc protected amino acids containing standard side-chain protecting groups, except for the residue at position 1 (e.g., Boc-L-Tyr(OtBu)-OH or Boc-L-His(Boc)-OH), the lysine residue at position 17 (Fmoc-L-Lys(Mtt)-OH), and the lysine residues at positions 24, 28, 32, or 40 (Fmoc-L-Lys(ivDde)-OH). Before each coupling step, Fmoc groups are removed using 20% piperidine in DMF (two treatments of 8 minutes each). To initiate coupling, equimolar amounts of Fmoc amino acid (0.3 M in DMF), diisopropylcarbodiimide (0.9 M in DCM), and Oxyma (0.9 M in DMF) are combined at a 9-fold molar excess over the theoretical peptide loading, with the reaction maintained at 60° C. Coupling times vary: standard Fmoc-amino acids are coupled for 40 minutes, whereas Fmoc-α-methylated amino acids require 3 hours, as do Fmoc-amino acids being coupled onto an α-methylated residue. Extended coupling times of 6 hours apply for Fmoc-amino acids at positions 5 and 6, while residues at positions 1-4 undergo coupling for 3 hours. Upon completion of the peptide backbone synthesis, the resin undergoes thorough washing with DCM to remove residual DMF.

### Procedure for Assembly of Linker-Fatty Acid Moiety

After peptide backbone synthesis, the Mtt protecting group on the lysine at position 17 is selectively removed by treatment with 30% hexafluoroisopropanol in DCM (four treatments of 30 minutes each), followed by thorough washing with DCM. The linker-fatty acid moiety is assembled using a coupling approach similar to the previous steps, employing selected N-Fmoc protected amino acid derivatives as needed. These may include 2-[2-(2-Fmoc-amino-ethoxy)-ethoxy]-acetic acid (Fmoc-AEEA-OH), Fmoc-glutamic acid α-t-butyl ester (Fmoc-Glu(OH)-OtBu), Nα-tert-butoxycarbonyl-Nε-Fmoc-lysine (Boc-L-Lys(Fmoc)-OH), and 18-(tert-butoxy)-18-oxo-octadecanoic acid. Each derivative is coupled in amounts ranging from 4 to 9 equivalents relative to resin loading (0.15-0.3 M in DMF) in the presence of diisopropylcarbodiimide (9 equivalents, 0.9 M in DCM) and Oxyma (9 equivalents, 0.9 M in DMF). The coupling reactions proceed at 60° C. for 6 hours. Prior to each coupling step, Fmoc groups are removed using 20% piperidine in DMF (two treatments of 8 minutes each).

### Procedure for Assembly of Linker-Fc Conjugation Moiety

After assembling the linker-fatty acid moiety, the ivDde protecting group on the lysine residues at positions 24, 28, 32, or 40 is selectively removed by treatment with 3% hydrazine in DMF (four treatments of 3 minutes each). The resin is thoroughly washed with DMF, DCM, and isopropyl alcohol to ensure complete removal of any residual hydrazine. If a linker is present, it is constructed using a similar approach to previous coupling steps, utilizing selected N-Fmoc protected amino acid derivatives as needed. These may include Fmoc-N-amido-PEG₂₄-Acid, 2-[2-(2-Fmoc-amino-ethoxy)-ethoxy]-acetic acid (Fmoc-AEEA-OH), or Fmoc-amino acids with standard protecting groups. Each derivative is coupled in amounts ranging from 2 to 9 equivalents relative to resin loading (0.15-0.3 M in DMF) in the presence of diisopropylcarbodiimide (9 equivalents, 0.9 M in DCM) and Oxyma (9 equivalents, 0.9 M in DMF). The coupling reactions proceed at 60° C. for 2 to 6 hours, as required. Prior to each coupling step, Fmoc groups are removed using 20% piperidine in DMF (two treatments of 8 minutes each).

### Coupling of BEA

Once the desired linker is assembled, the terminal Fmoc group is removed using 20% piperidine in DMF (two treatments of 8 minutes each). Coupling to ‘BEA’ is then performed by adding 4-(bis(2-((((9H-fluoren-9-yl)methoxy)carbonyl)amino)ethyl)amino)-4-oxobutanoic acid (Fmoc2-BEA-OH, 6 equivalents in DMF), diisopropylcarbodiimide (9 equivalents, 0.9 M in DCM), and Oxyma (9 equivalents, 0.9 M in DMF). The reaction is maintained at 60° C. for 4 hours. Upon completion, both Fmoc groups are removed with 20% piperidine in DMF (two treatments of 8 minutes each) before introducing the desired thiol-reactive conjugation moieties, as detailed below:

### For Peptide Intermediates Containing BEA-(MalDap)₂

To the resin-bound diamine, (S)-3-((tert-butoxycarbonyl)amino)-2-(2,5-dioxo-2,5-dihydro-1H-pyrrol-1-yl)propanoic acid (N-Mal-L-Dap(Boc)-OH, 3.6 equivalents in DMF), diisopropylcarbodiimide (9 equivalents, 0.9 M in DCM), and Oxyma (9 equivalents, 0.9 M in DMF) are introduced, allowing the reaction to proceed at room temperature for 3 hours.

### For Peptide Intermediates Containing BFA-(MSPT)₂

To the resin-bound diamine, N,N-Diisopropylethylamine (1.7 equivalents) and 2,5-dioxopyrrolidin-1-yl 2-(2-(2-(4-(5-(methylsulfonyl)-1H-tetrazol-1-yl)phenoxy)ethoxy)ethoxy)acetate (MSPT-NHS Ester, 3 equivalents in DMF) are introduced, allowing the reaction to proceed at room temperature for 2.5 hours.

### For Peptide Intermediates Containing BEA-(AcBr)₂

N,N-Diisopropylethylamine (1.7 equivalents) and 2,5-dioxopyrrolidin-1-yl 2-bromoacetate (4.1 equivalents in DMF) are added to the resin-bound diamine, and the reaction allowed to proceed at room temperature for 2.5 hours.

### For Peptide Intermediates Containing BFA-(OD)₂

N,N-Diisopropylethylamine (1.7 equivalents) and 2,5-dioxopyrrolidin-1-yl 2-(2-(2-(4-(5-(methylsulfonyl)-1,3,4-oxadiazol-2-yl)phenoxy)ethoxy)ethoxy)acetate (OD-NHS Ester, 3 equivalents in DMF) are added to the resin-bound diamine, and the reaction allowed to proceed at room temperature for 2.5 hours.

### For Peptide Intermediates Containing BEA-(β-MalDap)₂

To the resin-bound diamine, N,N-Diisopropylethylamine (1.7 equivalents) and 2,5-Dioxopyrrolidin-1-yl (S)-2-((tert-butoxycarbonyl)amino)-3-(2,5-dioxo-2,5-dihydro-1H-pyrrol-1-yl)propanoate (Boc-L-Dap(Mal)-NHS Ester, 3 equivalents in DMF) were added, and the mixture allowed to proceed at room temperature overnight.

### For Peptide Intermediates Containing a Single MalDap (for Conjugation to Fc-eCys)

To the resin-bound amine (no BEA is present), (S)-3-((tert-butoxycarbonyl)amino)-2-(2,5-dioxo-2,5-dihydro-1H-pyrrol-1-yl)propanoic acid (N-Mal-L-Dap(Boc)-OH, 1.8 equivalents in DMF), diisopropylcarbodiimide (9 equivalents, 0.9 M in DCM), and Oxyma (9 equivalents, 0.9 M in DMF) are introduced, allowing the reaction to proceed at room temperature for 3 hours.

### For Peptide Intermediates Containing a Single MSPT (for Conjugation to Fc-eCys)

To the resin-bound amine (no BEA is present), 2-(2-(2-(4-(5-(methylsulfonyl)-1H-tetrazol-1-yl)phenoxy)ethoxy)ethoxy)acetic acid (MSPT-acid, 1.8 equivalents in DMF), diisopropylcarbodiimide (9 equivalents, 0.9 M in DCM), and Oxyma (9 equivalents, 0.9 M in DMF) are introduced, allowing the reaction to proceed at room temperature for 3 hours.

### Cleavage and Purification of Peptide Intermediates

After completion of the on-resin synthetic steps detailed above, the peptide resin is washed with DCM and then thoroughly air-dried. The resin is treated with 10 mL of cleavage cocktail (trifluoroacetic acid:water:triisopropylsilane, 85:5:10 v/v) for 2.5 hours at room temperature. The resin is filtered off, washed twice each with 2 mL of neat TFA, and the combined filtrates are treated with 5-fold (by volume) cold diethyl ether (−20° C.) to precipitate the crude peptide. The peptide/ether suspension is then centrifuged at 5500 rpm for 2 min to form a solid pellet, the supernatant is decanted, and the solid pellet is triturated with ether two additional times and dried under nitrogen. The crude peptide is solubilized in 20% acetonitrile/20% acetic acid/60% water and purified by RP-HPLC on a Luna 5 μm Phenyl-Hexyl Preparative Column (21×250 mm, Phenomenex) with linear gradients of 100% acetonitrile in a 0.1% TFA/water buffer system (e.g. 30-50% acetonitrile over 75 min). Product purity is assessed using analytical RP-HPLC and the pooling criteria is >95%. Pooled fractions are frozen and concentrated by lyophilization to yield the peptide intermediate as a TFA salt.

### Synthesis of 4-(bis(2-((((9H-fluoren-9-yl)methoxy)carbonyl)amino)ethyl)amino)-4-oxobutanoic Acid (Fmoc2-BEA-OH)

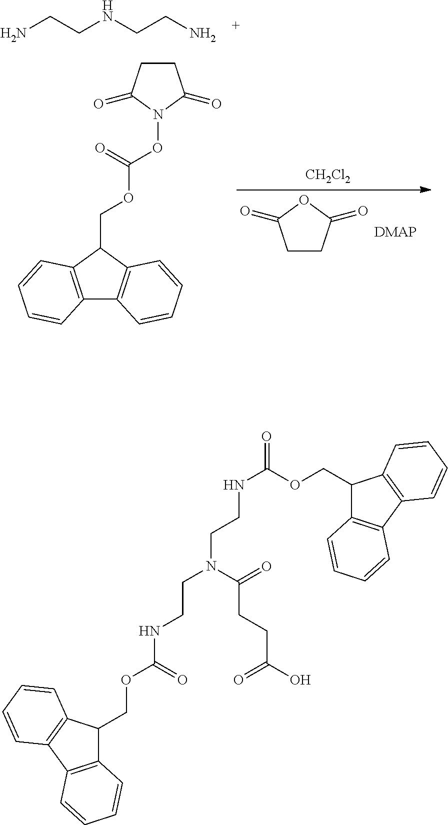

A solution of N-(9-fluorenylmethoxycarbonyloxy) succinimide (19.4 g, 56.4 mmol) in dichloromethane (80 mL) was added over 45 min to a solution of diethylenetriamine (3.1 mL, 28 mmol) in dichloromethane (30 mL) cooled at −78° C. After 2 hours, the mixture was warmed to ambient temperature, and succinic anhydride (9.91 g, 98 mmol) and DMAP (691 mg, 5.60 mmol) were added. The mixture was stirred at ambient temperature for 15 hours, then the pH adjusted to 5 by slow addition of 1N HCl (˜20 mL). The phases were separated and the aqueous layer extracted with DCM (2×100 mL). The combined organic layers were washed with saturated aqueous NaCl, then dried over magnesium sulfate. The solvent was removed under reduced pressure and purification by silica gel chromatography (660 g; DCM (5 min) then 5% MeOH/DCM (15 min) then 10% MeOH/DCM (25 min) afforded the title product (911.84 g, 65%) as a white powder. ES/MS m/z: 648 (M+1).

### Synthesis of 2,5-dioxopyrrolidin-1-yl 2-(2-(2-(4-(5-(methylsulfonyl)-1H-tetrazol-1-yl)phenoxy)ethoxy)ethoxy)acetate (MSPT-NHS Ester)

### Step 1

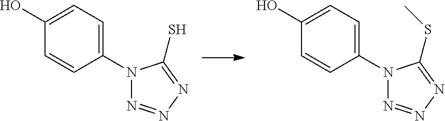

4-(5-mercapto-1H-tetrazol-1-yl)phenol (4.00 g, 20.6 mmol) was dissolved in tetrahydrofuran (50 mL), and the mixture cooled in an ice-bath, prior to the addition of NN-diisopropylethylamine (4.31 g, 33.3 mmol). A suspension formed after stirring for 10 minutes. Iodomethane (1.54 mL, 24.7 mmol) was added dropwise via syringe over 1 min. The reaction mixture was stirred for 20 minutes with cooling in an ice-bath, then at room temperature for 12 hours. The mixture was diluted with EtOAc (100 mL), and washed with saturated aqueous NH₄Cl (2×50 mL). The organic layer was separated, then dried over sodium sulfate, filtered and concentrated in vacuo to provide 4-(5-(methylthio)-1H-tetrazol-1-yl)phenol (4.2 g, 93% yield) that can be used directly in the next step without further purification. LCMS—mz=209 (M+1).

### Step 2

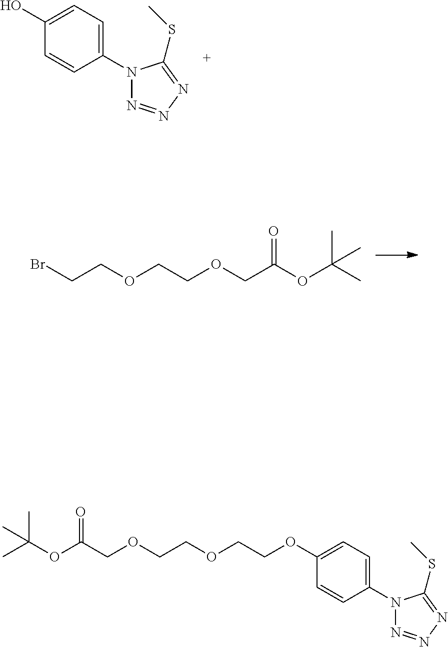

A 200 mL pressure vessel was charged with 4-(5-(methylthio)-1H-tetrazol-1-yl)phenol (2.50 g, 11.4 mmol), tert-butyl 2-(2-(2-bromoethoxy)ethoxy)acetate (4.33 g, 14.8 mmol) and acetone (60 mL). Potassium carbonate was added (3.15 g, 22.8 mmol), and the vessel sealed and heated at 80° C. for 8 hours with vigorous stirring. The reaction mixture was cooled to room temperature and filtered to remove potassium carbonate, then washed with acetone/DCM/EtOAc (30 mL each). The filtrate was concentrated in vacuo to provide crude material, which was purified by flash chromatography (80 g, 100% DCM for 5 minutes, then gradient to 100% EtOAc over 20 minutes). The product tert-butyl 2-(2-(2-(4-(5-(methylthio)-1H-tetrazol-1-yl)phenoxy)ethoxy)ethoxy)acetate (3.98 g, 85% yield) was isolated as a white powder. LCMS—mz=411 (M+1).

### Step 3

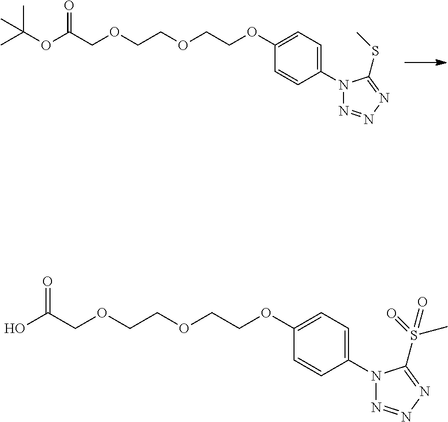 [SMILES: CSc1nnnn1-c1ccc(OCCOCCOCC(=O)OC(C)(C)C)cc1 ; CS(=O)(=O)c1nnnn1-c1ccc(OCCOCCOCC(=O)O)cc1]

Tert-butyl 2-(2-(2-(4-(5-(methylthio)-1H-tetrazol-1-yl)phenoxy)ethoxy)ethoxy)acetate (3.98 g, 9.21 mmol) was dissolved in ethanol (100 mL) and cooled to 5-10° C. in an ice-water bath, prior to the addition of 30% aqueous hydrogen peroxide (19.0 mL, 184 mmol), followed by ammonium molybdate (VI) tetrahydrate (1.14 g, 0.921 mmol). The reaction mixture was stirred at room temperature for 4 hours in an ice bath, then at room temperature for 12 hours. The mixture was diluted with DCM (150 mL) and then washed with brine. The organic phase was separated, dried over sodium sulfate and concentrated in vacuo to dryness. Purification by flash column chromatography (80 g silica, 100% DCM for 5 minutes, then gradient to 100% EtOAc over 20 minutes) provided 2-(2-(2-(4-(5-(methylsulfonyl)-1H-tetrazol-1-yl)phenoxy)ethoxy)ethoxy)acetic acid (MSPT Acid, 3.00 g, 80% yield) as a thick oil. LCMS—mz=385 (M−1)

### Step 4

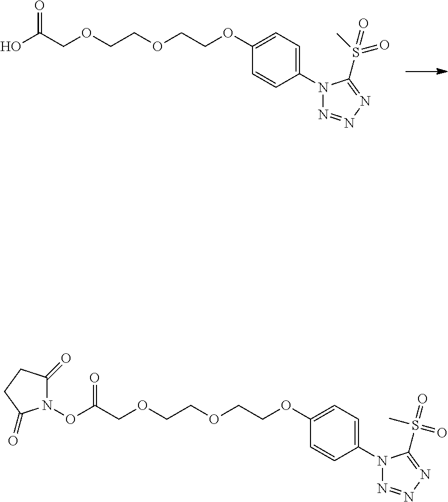 [SMILES: CS(=O)(=O)c1nnnn1-c1ccc(OCCOCCOCC(=O)O)cc1 ; CS(=O)(=O)c1nnnn1-c1ccc(OCCOCCOCC(=O)ON2C(=O)CCC2=O)cc1]

1-Hydroxypyrrolidine-2,5-dione (1.33 g, 1.6 Eq, 11.6 mmol) was added to a solution of 2-(2-(2-(4-(5-(methylsulfonyl)-1H-tetrazol-1-yl)phenoxy)ethoxy)ethoxy)acetic acid (2.80 g, 7.25 mmol) in DCM (50 mL) and THF (70 mL). EDCI (1.60 g, 10.3 mmol) was added in one portion, upon which the solution became a cloudy mixture. Additional DCM (20 mL) was added to bring the mixture into solution again, followed by stirring at room temperature for 12 hours. The solvent was removed in vacuo to provide crude material as a white foam. Purification by flash column chromatography (80 g, 100% DCM for 5 minutes, then gradient to 100% EtOAc over 20 minutes) afforded 2,5-dioxopyrrolidin-1-yl 2-(2-(2-(4-(5-(methylsulfonyl)-1H-tetrazol-1-yl)phenoxy)ethoxy)ethoxy)acetate (MSPT-NHS Ester, 2.61 g, 65% yield) as low melting solid. LCMS—mz=484 (M+1)

### Synthesis of 2,5-dioxopyrrolidin-1-yl 2-(2-(2-(4-(5-(methylsulfonyl)-1,3,4-oxadiazol-2-yl)phenoxy)ethoxy)ethoxy)acetate (OD-NHS Ester)

### Step 1

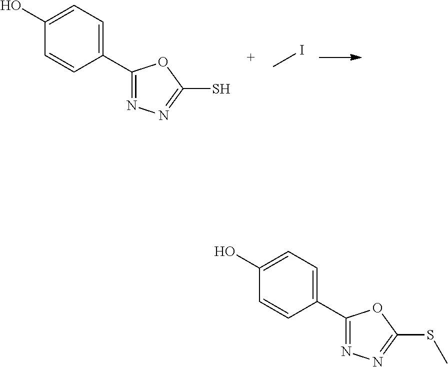

4-(5-Mercapto-1,3,4-oxadiazol-2-yl)phenol (3.00 g, 15.4 mmol) was dissolved in THF (50 mL), and cooled to 0° C. in an ice water bath. N,N-Diisopropylethylamine (3.46 mL, 2.60 g, 20.1 mmol) was added, resulting in a cloudy solution. The mixture was stirred for 5 minutes in the ice bath, then iodomethane (2.85 g, 20.1 mmol) was added drop-wise via syringe over a period of 1 minute. Upon addition, the mixture turned clear after 5 minutes. The cooling bath was removed and the mixture stirred at room temperature for 2 hours, after which it was diluted with dichloromethane (100 mL) and washed with saturated aqueous NH4Cl (pH was adjusted to ˜5 by adding citric acid solution, 2×50 mL). The organic layer was separated, dried over sodium sulfate, and concentrated in vacuo to dryness to afford 4-(5-methylsulfanyl-1,3,4-oxadiazol-2-yl)phenol (520 mg, 97% yield) as a pale-yellow solid that was used in the next step without further purification. LC-MS—mz=209 (M+1).

### Step 2

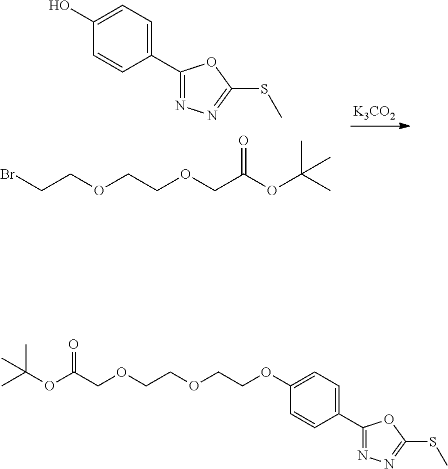

4-(5-(Methylthio)-1,3,4-oxadiazol-2-yl)phenol (3.3 g, 1 Eq, 15 mmol) and acetone (60 mL) were added to a 200 mL pressure vessel. To this solution was added tert-butyl 2-(2-(2-bromoethoxy)ethoxy)acetate (5.5 g, 20 mmol) and potassium carbonate (4.2 g, 30 mmol). The pressure vessel was sealed and heated at 70° C. for 5 hours with vigorous stirring. After cooling to room temperature, the mixture was filtered to remove solid potassium carbonate, washing with EtOAc/DCM. The filtrate was concentrated in vacuo to dryness and purified by normal phase flash column chromatography (80 g silica gold, 100% DCM for 5 minutes, then gradient to 100% EtOAc over 20 minutes). Product-containing fractions were concentrated in vacuo to afford tert-butyl 2-(2-(2-(4-(5-(methylthio)-1,3,4-oxadiazol-2-yl)phenoxy)ethoxy)ethoxy)acetate (4.8 g, 74% yield) as a white solid. LC-MS—mz=411 (M+1).

### Step 3

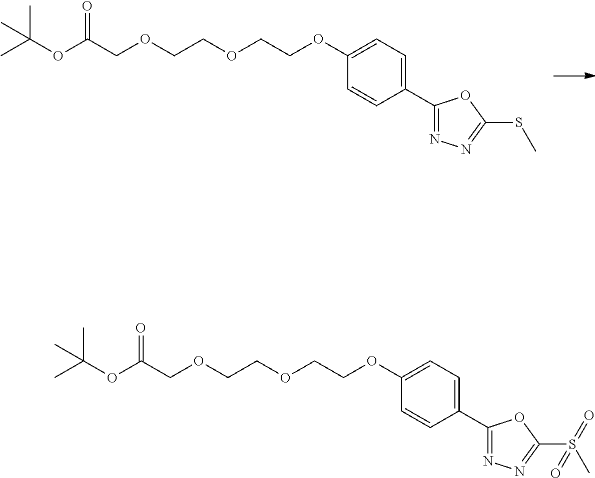

Tert-butyl 2-(2-(2-(4-(5-(methylthio)-1,3,4-oxadiazol-2-yl)phenoxy)ethoxy)ethoxy)acetate (5.20 g, 12.7 mmol) was dissolved in 100 mL of ethanol, and cooled to 5-10° C. in an ice-water bath, prior to the addition of 30% hydrogen peroxide (10 mL, 97 mmol), followed by ammonium molybdate (VI) tetrahydrate (501 mg, 0.405 mmol). After two hours of vigorous stirring, an additional 15 mL of 30% hydrogen peroxide and 1 g of Ammonium molybdate (VI) tetrahydrate were added. The reaction mixture was stirred for another 6 hours, then diluted with 150 mL of DCM, and washed with brine. The organic phase was separated, dried over sodium sulfate and concentrated in vacuo to dryness. The residue was triturated with methanol to provide an initial portion of the product. The solvent was then removed from the mother liquid under reduced pressure. Purification by flash column chromatography (40 g, 100% DCM for 3 minutes, then gradient to 100% EtOAc over 20 minutes) provided additional product as a white solid. Combination of both product portions yielded tert-butyl 2-(2-(2-(4-(5-(methylsulfonyl)-1,3,4-oxadiazol-2-yl)phenoxy)ethoxy)ethoxy)acetate (5.3 g, 90% yield) as a white solid. LC-MS—mz=387 (M-tBu).

### Step 4

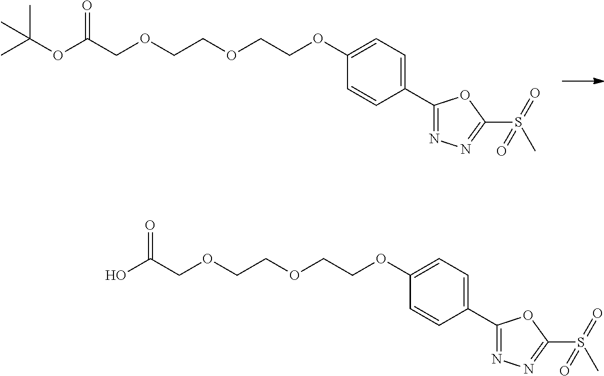 [SMILES: CC(C)(C)OC(=O)COCCOCCOc1ccc(-c2nnc(S(C)(=O)=O)o2)cc1 ; CS(=O)(=O)c1nnc(-c2ccc(OCCOCCOCC(=O)O)cc2)o1]

To a solution of tert-butyl 2-(2-(2-(4-(5-(methylsulfonyl)-1,3,4-oxadiazol-2-yl)phenoxy)ethoxy)ethoxy)acetate (5.60 g, 12.0 mmol) in DCM (60 mL) was added 2,2,2-trifluoroacetic acid (20 mL, 12.0 mmol). The reaction mixture was stirred at room temperature for 2 hours then concentrated under vacuo to afford a thick residue, which was purified by normal phase flash column chromatography (80 g silica gold column, 100% DCM for 3 minutes, then gradient to 100% EtOAc over 20 minutes). Combination of product-containing fractions and concentration in vacuo yielded 2-(2-(2-(4-(5-(methylsulfonyl)-1,3,4-oxadiazol-2-yl)phenoxy)ethoxy)ethoxy)acetic acid (4.12 g, 82% yield). LCMS—mz=387 (M+1).

### Step 5

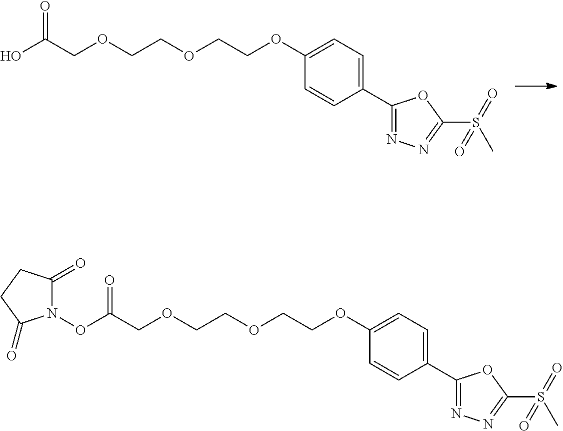 [SMILES: CS(=O)(=O)c1nnc(-c2ccc(OCCOCCOCC(=O)O)cc2)o1 ; CS(=O)(=O)c1nnc(-c2ccc(OCCOCCOCC(=O)ON3C(=O)CCC3=O)cc2)o1]

2-(2-(2-(4-(5-(Methylsulfonyl)-1,3,4-oxadiazol-2-yl)phenoxy)ethoxy)ethoxy)acetic acid (3.00 g, 7.38 mmol) and 1-hydroxypyrrolidine-2,5-dione (1.19 g, 10.3 mmol) were dissolved in DCM (50 mL) and THF (70 mL). To this solution was added 3-(((ethylimino)methylene)amino)-N,N-dimethylpropan-1-amine (EDCI, 1.60 g, 10.3 mmol). Upon addition, the solution turned cloudy and additional DCM (20 mL) was added to bring the mixture into solution again, followed by stirring at room temperature for 12 hours. The reaction mixture was concentrated under reduced pressure, and the resulting residue dissolved in DCM purified by normal phase flash column chromatography (80 g silica gold 100% DCM for 5 minutes, then gradient to 100% EtOAc over 20 minutes). Concentration of product-containing fractions afforded 2,5-dioxopyrrolidin-1-yl-2-(2-(2-(4-(5-(methylsulfonyl)-1,3,4-oxadiazol-2-yl)phenoxy)ethoxy)ethoxy)acetate (2.61 g, 65% yield). LCMS—mz=484 (M+1).

### Fe Re-Bridging

The Fc is reduced by adding 1.5-2 eq. of freshly prepared TCEP stock in water. 258 μl of 7 mM TCEP solution is added to 9 mL of 111 μM Fc at pH 7.5. The solution was then incubated at 37° C. for 1 h and the reduction is verified by analyzing in LC-MS. Reduced Fc is then used directly for conjugation with the peptide. For efficient conjugation, the reaction is performed at 21.2 μM Fc concentration with a suitable excess (˜1.4 eq.) of the peptide, as follows: The reduced Fc (˜9 mL) is transferred to a 50 mL vial, to which 50 mM acetate buffer (˜29 mL) pH 5.6 and 6 mL of ACN are added. The solution is gently mixed and left at 4° C. before adding the peptide, as a 0.5 mM stock solution in 30% Acetonitrile in water. Reaction progress is monitored by LC-MS and typically completes within 30 minutes. To promote maleimide ring hydrolysis, the reactions is adjusted to pH 8.0 by addition of 1M Tris buffer at pH 8.0. The desired product is isolated after purification through IEX.

eCys Conjugation for Alternative Peptide Attachment Sites

The Fc with appropriate engineered cysteine was first reduced in the presence of 8-10 molar equivalents of dithiothreitol (DTT) for 2 hours at 37° C. Following reduction, the sample was passed through a Zeba spin desalting column (Thermo Fisher Scientific) or alternatively size exclusion chromatography to remove the reducing agent and cysteine or glutathione caps that were attached to the engineered cysteines during expression. Dehydroascorbic acid (DHAA), 1-5 molar equivalent was then added, and the sample was kept at room temperature for 0.5-1 hour to reform the hinge disulfide. Following oxidation, the sample was again passed through a Zeba spin column (Thermo Fisher Scientific), or alternatively a size exclusion chromatography column to remove the oxidizing agent and buffer exchange the sample into the desired buffer for conjugation.

eCys Conjugation for a Single Peptide

To generate an Fc with a single eCys site, a 1:1 mixture of an Fc with the desired site was mixed with the Fc containing the F405L and R409K mutations in the presence of 8-10 molar equivalents of dithiothreitol(DTT) and incubated at 37° C. for 2 hours. Following reduction, the reducing agent and cysteine or glutathione caps that were attached to the engineered cysteines during expression were removed using either a desalting column or size exclusion chromatography. Dehydroascorbic acid (DHAA), 1-5 molar equivalent was then added, and the sample was kept at room temperature for 0.5-1 hour to reform the hinge disulfide with the preference for hetero Fc formation driven by the F405L and R409K mutations in the one Fc molecule in the mixture. Following oxidation, the sample was again passed through a Zeba spin column (Thermo Fisher Scientific), or alternatively a size exclusion chromatography column to remove the oxidization agent and buffer exchange the sample into the desired buffer for conjugation.

Conjugation to Single or Double eCys Fc Constructs

For efficient conjugation to eCys variants, the reaction is performed at 21.2 μM Fc concentration with a suitable excess (˜1.4 eq for single eCys or ˜2.8Eq for double eCys) of the peptide, as follows: Fc (˜9 mL) is transferred to a 50 mL vial, to which 50 mM acetate buffer (˜29 mL) pH 5.6 and 6 mL of ACN are added. The solution is gently mixed and left at 4° C. before adding the peptide, as a 0.5 mM stock solution in 30% Acetonitrile in water. Reaction progress is monitored by LC-MS and typically completes within 30 minutes. To promote maleimide ring hydrolysis, the reactions is adjusted to pH 8.0 by addition of 1M Tris buffer at pH 8.0. The desired product is isolated after purification through IEX.

TABLE 3
Exemplary Therapeutic Polypeptide Fc conjugates

| | | | | | | Fc |
|---|---|---|---|---|---|---|
| | | | | Table 1 | Table 2 | region |
| Compound | Figure | (SEQ ID) | Therapeutic Polypeptide Fc Conjugates | (SEQ ID) | (SEQ ID) | (SEQ ID) |
| 1 | 2 | 459 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(PEG₂₄-BEA-MalDap₂-Fc)-NH₂ | | | |
| 2 | | 460 | Y-Aib-EGT-αMeF(2F)-TSD-4Pal-SI-αMeL-LD-Orn-K(2-[2-(2-amino- | 2 | 188 | 935 |
| | | | ethoxy)-ethoxy]-acetyl)₂-(γGlu)-CO-(CH₂)₁₆-CO₂H)AQ-Aib-EFIe-αMeY- | | | |
| | | | LIEGSPSSGAPPPSK(PEG₂₄-BEA-MalDap₂-Fc)-NH₂ | | | |
| 3 | | 461 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSK(BEA-MalDap₂-Fc)-NH₂ | | | |
| 4 | | 462 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH2)₁₆-CO₂H)AQ-Aib-EFIe-αMeY- | | | |
| | | | LIEGSPSSGAPPPSGGGGSK(BEA-MalDap₂-Fc)-NH₂ | | | |
| 5 | | 463 | Y-Aib-EGT-αMeF(2F)-TSD-4Pal-SI-αMeL-LDEK(2-[2-(2-amino-ethoxy)- | 1 | 187 | 935 |
| | | | cthoxy]-acetyl)₂-(γGlu)-CO-(CH₂)₁₆-CO₂H)AQ-Aib-EFIe-αMeY- | | | |
| | | | LIEGSPSSGAPPPS-(2-[2-(2-amino-ethoxy)-ethoxy]-acetyl)₈-K(BEA- | | | |
| | | | MalDap₂-Fc)-NH₂ | | | |
| 6 | | 464 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPS-(2-[2-(2-amino-ethoxy)-ethoxy]-acetyl)₄-K(BEA- | | | |
| | | | MalDap2-Fc)-NH₂ | | | |
| 7 | | 465 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPS-PEG₂₄-K(BEA-MalDap₂-Fc)-NH₂ | | | |
| 8 | | 466 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(2-[2-(2-amino-ethoxy)-ethoxy]-acetyl)₄-BEA- | | | |
| | | | MalDap₂-Fc)-NH₂ | | | |
| 9 | | 467 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(2-[2-(2-amino-ethoxy)-ethoxy]-acetyl)₄- | | | |
| | | | GPAPAPAPAPAPA-BEA-MalDap₂-Fc)-NH₂ | | | |
| 10 | | 468 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc)-NH₂ | | | |
| 11 | | 469 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(2-[2-(2-amino-ethoxy)-ethoxy]-acetyl)₄- | | | |
| | | | GPAPAPAPAPAPAPAPA-BEA-MalDap₂-Fc)-NH₂ | | | |
| 12 | | 470 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(GPAPAPAPAPAPAPAPA-BEA-MalDap₂-Fc)-NH₂ | | | |
| 13 | | 471 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(2-[2-(2-amino-ethoxy)-ethoxy]-acetyl)₄- | | | |
| | | | GKAAAEKAAAEKAAAE-BEA-MalDap₂-Fc)NH₂ | | | |
| 14 | | 472 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(LEAEAAAKEAAAKEAAAKEAAAKALE-BEA- | | | |
| | | | MalDap₂-Fc)-NH₂ | | | |
| 15 | | 473 | Y-Aib-EGT-αMeF(2F)-TSD-4Pal-SI-αMeL-LDEK(2-[2-(2-amino-ethoxy)- | 4 | 190 | 935 |
| | | | ethoxy]-acetyl)₂-(γGlu)-CO-(CH₂)₁₆-CO₂H)AQ-Aib-EFIE-αMeY- | | | |
| | | | LIEGSPSSGAPPPS-K(PEG₂₄-BEA-MalDap2-Fc)-NH₂ | | | |
| 16 | | 474 | Y-Aib-EGT-αMeF(2F)-TSD-4Pal-Aib-I-αMeL-LDEK(2-[2-(2-amino- | 5 | 191 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPS-K(PEG₂₄-BEA-MalDap₂-Fc)-NH₂ | | | |
| 17 | | 475 | Y-Aib-EGT-αMeF(2F)-TSD-4Pal-Aib-S-αMeL-LDEK(2-[2-(2-amino- | 6 | 192 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPS-K(PEG₂₄-BEA-MalDap₂-Fc)-NH₂ | | | |
| 18 | | 476 | Y-Aib-EGT-αMeF(2F)-TSD-4Pal-SI-αMeL-LEEK(2-[2-(2-amino-ethoxy)- | 7 | 193 | 935 |
| | | | ethoxy]-acetyl)₂-(γGlu)-CO-(CH₂)₁₆-CO₂H)AQ-Aib-EFIE-αMeY- | | | |
| | | | LIEGSPSSGAPPPS-K(PEG₂₄-BEA-MalDap₂-Fc)-NH₂ | | | |
| 19 | | 477 | Y-Aib-EGT-αMeF(2F)-TSDV-Aib-I-αMeL-LD-EK(2-[2-(2-amino-ethoxy)- | 8 | 194 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPS-K(PEG₂₄-BEA-MalDap₂-Fc)-NH₂ | | | |
| 20 | | 478 | Y-Aib-EGT-αMeF(2F)-TSDV-Aib-I-αMeL-LD-Orn-K(2-[2-(2-amino- | 9 | 195 | 935 |
| | | | ethoxy)-ethoxy]-acetyl)₂-(γGlu)-CO-(CH₂)₁₆-CO₂H)AQ-Aib-EFIe-αMeY- | | | |
| | | | LIEGSPSSGAPPPS-K(PEG₂₄-BEA-MalDap₂-Fc)-NH₂ | | | |
| 21 | | 479 | Y-Aib-EGT-αMeF(2F)-TSDV-Aib-I-αMeL-LD-Orn-K(2-[2-(2-amino- | 10 | 196 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPS-K(PEG₂₄-BEA-MalDap₂-Fc)-NH₂ | | | |
| 22 | | 480 | Y-Aib-EGT-αMeF(2F)-TSD-4Pal-SI-αMeL-LDEK(2-[2-(2-amino-ethoxy)- | 11 | 197 | 935 |
| | | | ethoxy]-acetyl)₂-(γGlu)-CO-(CH₂)₁₆-CO₂H)AQ-Aib-EFIK(PEG₂₄-BEA- | | | |
| | | | MalDap₂-Fc)-αMeY-LIEGSPSSGAPPPSGS-NH₂ | | | |
| 23 | | 481 | Y-Aib-EGT-αMeF(2F)-TSD-4Pal-SI-αMeL-LDEK(2-[2-(2-amino-ethoxy)- | 12 | 198 | 935 |
| | | | ethoxy]-acetyl)₂-(γGlu)-CO-(CH₂)₁₆-CO₂H)AQ-Aib-EFIe-αMeY-LIK(PEG₂₄- | | | |
| | | | BEA-MalDap₂-Fc)GSPSSGAPPPSGS-NH₂ | | | |
| 24 | | 482 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGGGGSK(BEA-MalDap₂-Fc)-NH₂ | | | |
| 25 | | 483 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGGGGSGGGGSK(BEA-MalDap₂-Fc)- | | | |
| | | | NH₂ | | | |
| 26 | | 484 | Y-Aib-EGT-αMeF(2F)-TSD-4Pal-SI-αMeL-LDEK(2-[2-(2-amino-ethoxy)- | 13 | 199 | 935 |
| | | | ethoxy]-acetyl)₂-(γGlu)-CO-(CH₂)₁₆-CO₂H)AQ-Aib-EFIe-αMeY- | | | |
| | | | LIEGGPGGGGSGGGGSGGGGSK(BEA-MalDap₂-Fc)-NH₂ | | | |
| 27 | | 485 | Y-Aib-EGT-αMeF(2F)-TSD-4Pal-SI-αMeL-LDEK(2-[2-(2-amino-ethoxy)- | 14 | 200 | 935 |
| | | | ethoxy]-acetyl)₂-(γGlu)-CO-(CH₂)₁₆-CO₂H)AQ-Aib-EFIe-αMeY- | | | |
| | | | LIEGSPGGGGSGGGGSGGGGSK(BEA-MalDap₂-Fc)-NH₂ | | | |
| 28 | | 486 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGAPAPAPAPAPAPK(BEA-MalDap₂-Fc)-NH₂ | | | |
| 29 | | 487 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGAPAPAPAPAPAPK(BEA-MalDap₂-Fc)-NH₂ | | | |
| 30 | | 488 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGEAAAKEAAAKEAAAKK(BEA-MalDap₂-Fc)-NH₂ | | | |
| 31 | | 489 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGEAAAKEAAAKEAAAKK(BEA-MalDap₂-Fc)- | | | |
| | | | NH₂ | | | |
| 32 | 3 | 490 | Y-Aib-EGT-αMeF(2F)-TSD-4Pal-SI-αMeL-LD-Orn-K(ϵKϵK-(γGlu)-CO- | 2 | 258 | 935 |
| | | | (CH₂)₁₆-CO₂H)AQ-Aib-EFIe-αMeY-LIEGSPSSGAPPPSK(PEG₂₄-BEA- | | | |
| | | | MalDap₂-Fc)-NH₂ | | | |
| 33 | | 491 | Y-Aib-EGT-αMeF(2F)-TSD-4Pal-SI-αMeL-LDEK(ϵKϵK-(γGlu)-CO- | 1 | 201 | 935 |
| | | | (CH₂)₁₆-CO₂H)AQ-Aib-EFIe-αMeY-LIEGSPSSGAPPPSK(PEG₂₄-BEA- | | | |
| | | | MalDap₂-Fc)-NH₂ | | | |
| 34 | | 492 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGAPAPAPAPK(BEA-MalDap₂-Fc)-NH₂ | | | |
| 35 | | 493 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGAPAPAPK(BEA-MalDap₂-Fc)-NH₂ | | | |
| 36 | | 494 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGGGGSGAPAPAPK(BEA-MalDap₂-Fc)- | | | |
| | | | NH₂ | | | |
| 37 | | 495 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSEAAAKEAAAKEAAAKEAAAKK(BEA- | | | |
| | | | MalDap₂-Fc)-NH₂ | | | |
| 38 | | 496 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGEAAAKEAAAKEAAAKK(BEA- | | | |
| | | | MalDap₂-Fc)-NH₂ | | | |
| 39 | | 497 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGGGGSGEAAAKEAAAKEAAAKK(BEA- | | | |
| | | | MalDap₂-Fc)-NH₂ | | | |
| 40 | | 498 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 41 | | 499 | Y-Aib-EGT-αMeF(2F)-TSD-4Pal-Aib-I-αMeL-LDEK(2-[2-(2-amino- | 16 | 203 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 42 | | 500 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 43 | | 501 | Y-Aib-EGT-αMeF(2F)-TSD-V-Aib-I-αMeL-LDEK(2-[2-(2-amino-ethoxy)- | 18 | 205 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSK(BEA-MalDap₂-Fc)-NH₂ | | | |
| 44 | | 502 | Y-Aib-EGT-αMeF(2F)-TSD-V-Aib-I-αMeL-LD-Orn-K(2-[2-(2-amino- | 19 | 206 | 935 |
| | | | ethoxy)-ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSK(BEA-MalDap₂-Fc)-NH₂ | | | |
| 45 | | 503 | Y-Aib-EGT-αMeF(2F)-TSD-V-Aib-I-αMeL-LD-Orn-K(2-[2-(2-amino- | 20 | 207 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 46 | | 504 | Y-Aib-EGT-αMeF(2F)-TSD-4Pal-SI-αMeL-LD-Orn-K(2-[2-(2-amino- | 21 | 208 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 47 | 4 | 505 | Y-Aib-EGT-αMeF(2F)-TSD-4Pal-Aib-I-αMeL-LD-Orn-K(2-[2-(2-amino- | 22 | 209 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 48 | | 506 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 49 | | 507 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGAPAPAPAPAPAPAPAPK(BEA-MalDap₂-Fc)- | | | |
| | | | NH₂ | | | |
| 50 | | 508 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGAPAPAPAPAPAPK(BEA-MalDap₂-Fc)- | | | |
| | | | NH₂ | | | |
| 51 | | 509 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGGGGSGAPAPAPAPAPAPK(BEA- | | | |
| | | | MalDap₂-Fc)-NH₂ | | | |
| 52 | | 510 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGQGGGGQGGGGQK(BEA-MalDap₂-Fc)-NH₂ | | | |
| 53 | | 511 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGQGGGGQGGGGQGGGGQK(BEA-MalDap₂-Fc)-NH₂ | | | |
| 54 | | 512 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGQGGGGQGGGGQGGGGQGGGGQK(BEA-MalDap₂-Fc)- | | | |
| | | | NH₂ | | | |
| 55 | | 513 | Y-Aib-EGT-αMeF(2F)-TSD-4Pal-SI-αMeL-LDEK(2-[2-(2-amino-ethoxy)- | 11 | 197 | 935 |
| | | | ethoxy]-acetyl)₂-(γGlu)-CO-(CH₂)₁₆-CO₂H)AQ-Aib-EFIK(2-[2-(2-amino- | | | |
| | | | ethoxy)-ethoxy]-acetyl)₄-BEA-MalDap₂-Fc)-αMeY-LIEGSPSSGAPPPSGS- | | | |
| | | | NH₂ | | | |
| 56 | | 514 | Y-Aib-EGT-αMeF(2F)-TSD-4Pal-SI-αMeL-LDEK(2-[2-(2-amino-ethoxy)- | 24 | 211 | 935 |
| | | | ethoxy]-acetyl)₂-(γGlu)-CO-(CH₂)₁₆-CO₂H)AQ-Aib-EFIE-αMeY-LIK(2-12- | | | |
| | | | (2-amino-ethoxy)-ethoxy]-acetyl)₄-BEA-MalDap₂-Fc)GSPSSGAPPPSGS- | | | |
| | | | NH₂ | | | |
| 57 | | 515 | Y-Aib-EGT-αMeF(2F)-TSD-4Pal-SI-αMeL-LDEK(2-[2-(2-amino-ethoxy)- | 24 | 211 | 935 |
| | | | ethoxy]-acetyl)₂-(γGlu)-CO-(CH₂)₁₆-CO₂H)AQ-Aib-EFIE-αMeY- | | | |
| | | | LIK(GGGGSGGGGSGGGGSGGGGSG-BEA-MalDap₂- | | | |
| | | | Fc)GSPSSGAPPPSGS-NH₂ | | | |
| 58 | | 516 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGAPAPAPAPAPAPK(BEA-MalDap₂-Fc)-NH₂ | | | |
| 59 | | 517 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGAPAPAPAPAPAPK(BEA-MalDap₂-Fc)- | | | |
| | | | NH₂ | | | |
| 60 | | 518 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGEAAAKEAAAKEAAAKK(BEA-MalDap₂-Fc)-NH₂ | | | |
| 61 | | 519 | Y-Aib-EGT-αMeF(2F)-TSD-4Pal-SI-αMeL-LDEK(2-[2-(2-amino-ethoxy)- | 15 | 202 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEAAAKEAAAKEAAAKK(BEA- | | | |
| | | | MalDap₂-Fc)-NH₂ | | | |
| 62 | | 520 | Y-Aib-EGT-αMeF(2F)-TSD-4Pal-Aib-I-αMeL-LDEK(2-[2-(2-amino- | 16 | 203 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGAPAPAPAPAPAPK(BEA-MalDap₂-Fc)-NH₂ | | | |
| 63 | | 521 | Y-Aib-EGT-αMeF(2F)-TSD-4Pal-Aib-I-αMeL-LDEK(2-[2-(2-amino- | 16 | 203 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGAPAPAPAPAPAPK(BEA-MalDap₂-Fc)- | | | |
| | | | NH₂ | | | |
| 64 | | 522 | Y-Aib-EGT-αMeF(2F)-TSD-4Pal-Aib-I-αMeL-LDEK(2-[2-(2-amino- | 16 | 203 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGEAAAKEAAAKEAAAKK(BEA-MalDap₂-Fc)-NH₂ | | | |
| 65 | | 523 | Y-Aib-EGT-αMeF(2F)-TSD-4Pal-Aib-I-αMeL-LDEK(2-[2-(2-amino- | 16 | 203 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEAAAKEAAAKEAAAKK(BEA- | | | |
| | | | MalDap₂-Fc)-NH₂ | | | |
| 66 | | 524 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGAPAPAPAPAPAPK(BEA-MalDap₂-Fc)-NH₂ | | | |
| 67 | | 525 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGAPAPAPAPAPAPK(BEA-MalDap₂-Fc)- | | | |
| | | | NH₂ | | | |
| 68 | | 526 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGEAAAKEAAAKEAAAKK(BEA-MalDap₂-Fc)-NH₂ | | | |
| 69 | | 527 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEAAAKEAAAKEAAAKK(BEA- | | | |
| | | | MalDap₂-Fc)-NH₂ | | | |
| 70 | | 528 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGAPAPAPAPAPAPK(BEA-MalDap2-Fc)-NH₂ | | | |
| 71 | | 529 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGAPAPAPAPAPAPK(BEA-MalDap₂-Fc)- | | | |
| | | | NH₂ | | | |
| 72 | | 530 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGEAAAKEAAAKEAAAKK(BEA-MalDap₂-Fc)-NH₂ | | | |
| 73 | | 531 | Y-Aib-EGT-αMeF(2F)-TSD-4Pal-SI-αMeL-LDEK(2-[2-(2-amino-ethoxy)- | 3 | 189 | 935 |
| | | | ethoxy]-acetyl)₂-(γGlu)-CO-(CH₂)₁₆-CO₂H)AQ-Aib-EFIe-αMeY- | | | |
| | | | LIEGGGAPAPAPAPAPAPGGGGSGGGGSK(BEA-MalDap₂-Fc)-NH₂ | | | |
| 74 | | 532 | Y-Aib-EGT-αMeF(2F)-TSD-4Pal-SI-αMeL-LDEK(2-[2-(2-amino-ethoxy)- | 11 | 197 | 935 |
| | | | ethoxy]-acetyl)₂-(γGlu)-CO-(CH₂)₁₆-CO₂H)AQ-Aib- | | | |
| | | | EFIK(SGGGGSGGGGSGGGGGPAPAPAPAPAPAG-BEA-MalDap₂-Fc)- | | | |
| | | | αMeY-LIEGSPSSGAPPPSGS-NH₂ | | | |
| 75 | | 533 | Y-Aib-EGT-αMeF(2F)-TSD-4Pal-SI-αMeL-LDEK(2-[2-(2-amino-ethoxy)- | 11 | 197 | 935 |
| | | | ethoxy]-acetyl)₂-(γGlu)-CO-(CH₂)₁₆-CO₂H)AQ-Aib- | | | |
| | | | EFIK(SGGGGSGGGGSGGGGGKAAAEKAAAEKAAAEG-BEA-MalDap₂- | | | |
| | | | Fc)-αMeY-LIEGSPSSGAPPPSGS-NH₂ | | | |
| 76 | | 534 | Y-Aib-EGT-αMeF(2F)-TSD-4Pal-SI-αMeL-LDEK(2-[2-(2-amino-ethoxy)- | 24 | 211 | 935 |
| | | | ethoxy]-acetyl)₂-(γGlu)-CO-(CH₂)₁₆-CO₂H)AQ-Aib-EFIE-αMeY- | | | |
| | | | LIK(SGGGGSGGGGSGGGGGPAPAPAPAPAPAG-BEA-MalDap₂- | | | |
| | | | Fc)GSPSSGAPPPSGS-NH₂ | | | |
| 77 | | 535 | Y-Aib-EGT-αMeF(2F)-TSD-4Pal-SI-αMeL-LDEK(2-[2-(2-amino-ethoxy)- | 24 | 211 | 935 |
| | | | ethoxy]-acetyl)₂-(γGlu)-CO-(CH₂)₁₆-CO₂H)AQ-Aib-EFIE-αMeY- | | | |
| | | | LIK(SGGGGSGGGGSGGGGGKAAAEKAAAEKAAAEG-BEA-MalDap₂- | | | |
| | | | Fc)GSPSSGAPPPSGS-NH₂ | | | |
| 78 | | 536 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(2-[2-(2-amino-ethoxy)-ethoxy]-acetyl)₄- | | | |
| | | | GPKPKPKPKPKPK-BEA-MalDap₂-Fc)-NH₂ | | | |
| 79 | | 537 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(2-[2-(2-amino-ethoxy)-ethoxy]-acetyl)₄- | | | |
| | | | GPEPEPEPEPEPE-BEA-MalDap₂-Fc)-NH₂ | | | |
| 80 | | 538 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGEPEPEPEPEPEPK(BEA-MalDap₂-Fc)-NH₂ | | | |
| 81 | | 539 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEPEPEPEPEPEPK(BEA-MalDap₂-Fc)- | | | |
| | | | NH₂ | | | |
| 82 | | 540 | Y-Aib-EGT-αMeF(2F)-TSD-4Pal-SI-αMeL-LEEK(2-[2-(2-amino-ethoxy)- | 17 | 204 | 935 |
| | | | ethoxy]-acetyl)₂-(γGlu)-CO-(CH₂)₁₆-CO₂H)AQ-Aib-EFIE-αMeY- | | | |
| | | | LIEGGGGGGGQGGGGQGGGGQGEPEPEPEPEPEPK(BEA-MalDap₂-Fc)- | | | |
| | | | NH₂ | | | |
| 83 | | 541 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGEPEPEPEPEPEPK(BEA-MalDap₂-Fc)-NH₂ | | | |
| 84 | | 542 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEPEPEPEPEPEPK(BEA-MalDap₂-Fc)- | | | |
| | | | NH₂ | | | |
| 85 | | 543 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGQGGGGQGGGGQGEPEPEPEPEPEPK(BEA-MalDap₂-Fc)- | | | |
| | | | NH₂ | | | |
| 86 | | 544 | Y-Aib-EGT-αMeF(2F)-TSD-4Pal-SI-αMeL-LEEK(2-[2-(2-amino-ethoxy)- | 7 | 193 | 935 |
| | | | ethoxy]-acetyl)₂-(γGlu)-CO-(CH₂)₁₆-CO₂H)AQ-Aib-EFIE-αMeY- | | | |
| | | | LIEGSPSSGAPPPS-(2-[2-(2-amino-ethoxy)-ethoxy]-acetyl)₂- | | | |
| | | | GEPEPEPEPEPEPK(BEA-MalDap₂-Fc)-NH₂ | | | |
| 87 | | 545 | Y-Aib-EGT-αMeF(2F)-TSD-4Pal-SI-αMeL-LEEK(2-[2-(2-amino-ethoxy)- | 7 | 193 | 935 |
| | | | ethoxy]-acetyl)₂-(γGlu)-CO-(CH₂)₁₆-CO₂H)AQ-Aib-EFIE-αMeY- | | | |
| | | | LIEGSPSSGAPPPS-(2-[2-(2-amino-ethoxy)-ethoxy]-acetyl)₄- | | | |
| | | | GEPEPEPEPEPEPK(BEA-MalDap₂-Fc)-NH₂ | | | |
| 88 | | 546 | Y-Aib-EGT-αMeF(2F)-TSD-4Pal-SI-αMeL-LE-Orn-K(2-[2-(2-amino- | 25 | 212 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPS-(2-[2-(2-amino-ethoxy)-ethoxy]-acetyl)₂- | | | |
| | | | GEPEPEPEPEPEPK(BEA-MalDap₂-Fc)-NH₂ | | | |
| 89 | | 547 | Y-Aib-EGT-αMeF(2F)-TSD-4Pal-SI-αMeL-LE-Orn-K(2-[2-(2-amino- | 25 | 212 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPS-(2-[2-(2-amino-ethoxy)-ethoxy]-acetyl)₄- | | | |
| | | | GEPEPEPEPEPEPK(BEA-MalDap₂-Fc)-NH₂ | | | |
| 90 | | 548 | Y-Aib-EGT-αMeF(2F)-TSD-4Pal-SI-αMeL-LEEK(2-[2-(2-amino-ethoxy)- | 7 | 193 | 935 |
| | | | ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc)-NH₂ | | | |
| 91 | | 549 | Y-Aib-EGT-αMeF(2F)-TSD-4Pal-SI-αMeL-LEEK(2-[2-(2-amino-ethoxy)- | 7 | 193 | 935 |
| | | | ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄- | | | |
| | | | GPEPEPEPEPEPE-BEA(MalDap₂-Fc)-NH₂ | | | |
| 92 | | 550 | Y-Aib-EGT-αMeF(2F)-TSD-4Pal-SI-αMeL-LEEK(2-[2-(2-amino-ethoxy)- | 7 | 193 | 935 |
| | | | ethoxy]-acetyl)₂-(γGlu)-CO-(CH₂)₁₆-CO2H)AQ-Aib-EFIE-αMeY- | | | |
| | | | LIEGSPSSGAPPPSK(2-[2-(2-amino-ethoxy)-ethoxy]-acetyl)₄- | | | |
| | | | GPEPEPEPEPEPEPEPE-BEA(MalDap₂-Fc)-NH₂ | | | |
| 93 | | 551 | Y-Aib-EGT-αMeF(2F)-TSD-4Pal-SI-αMeL-LE-Orn-K(2-[2-(2-amino- | 25 | 212 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA(MalDap₂-Fc)-NH₂ | | | |
| 94 | | 552 | Y-Aib-EGT-αMeF(2F)-TSD-4Pal-SI-αMeL-LE-Orn-K(2-[2-(2-amino- | 25 | 212 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄- | | | |
| | | | GPEPEPEPEPEPE-BEA(MalDap₂-Fc)-NH₂ | | | |
| 95 | | 553 | Y-Aib-EGT-αMeF(2F)-TSD-4Pal-SI-αMeL-LE-Orn-K(2-[2-(2-amino- | 25 | 212 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄- | | | |
| | | | GPEPEPEPEPEPEPEPE-BEA(MalDap₂-Fc)-NH₂ | | | |
| 96 | | 554 | Y-Aib-EGT-αMeF(2F)-TSD-4Pal-SI-αMeL-LD-Orn-K(AEEA2-γGlu- | 21 | 208 | 935 |
| | | | C18_OH)AQ-Aib-EFIE-αMeY- | | | |
| | | | LIEGGGGGGGSGGGGSGAPAPAPAPAPAPK(BEA-MalDap2-Fc)-NH2 | | | |
| 97 | | 555 | Y-Aib-EGT-αMeF(2F)-TSD-4Pal-Aib-I-αMeL-LD-Orn-K(2-[2-(2-amino- | 22 | 209 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc)-NH₂ | | | |
| 98 | | 556 | Y-Aib-EGT-αMeF(2F)-TSD-4Pal-SI-αMeL-LE-Orn-K(2-[2-(2-amino- | 23 | 210 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc)-NH₂ | | | |
| 99 | | 557 | Y-Aib-EGT-αMeF(2F)-TSD-4Pal-Aib-I-αMeL-LE-Orn-K(2-[2-(2-amino- | 26 | 213 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc)-NH₂ | | | |
| 100 | | 558 | Y-Aib-EGT-αMeF(2F)-TSD-4Pal-Aib-I-αMeL-LD-Orn-K(2-12-(2-amino- | 27 | 214 | 935 |
| | | | ethoxy)-ethoxy]-acetyl)₂-(γGlu)-CO-(CH₂)₁₆-CO₂H)AQ-Aib-EFIe-αMeY- | | | |
| | | | LIEGGGGGGGSGGGGSGAPAPAPAPAPAPK(BEA-MalDap₂-Fc)-NH₂ | | | |
| 101 | | 559 | Y-Aib-EGT-αMeF(2F)-TSD-4Pal-SI-αMeL-LD-Orn-K(2-[2-(2-amino- | 28 | 215 | 935 |
| | | | ethoxy)-ethoxy]-acetyl)₂-(γGlu)-CO-(CH₂)₁₆-CO₂H)AQ-Aib- | | | |
| | | | EFIK(GGGGSGGGGSGAPAPAPAPAPAPG-BEA-MalDap₂-Fc)-αMeY- | | | |
| | | | LIEGSPSSGAPPPSGS-NH₂ | | | |
| 102 | | 560 | Y-Aib-EGT-αMeF(2F)-TSD-4Pal-Aib-I-αMeL-LD-Orn-K(2-[2-(2-amino- | 29 | 216 | 935 |
| | | | ethoxy)-ethoxy]-acetyl)₂-(γGlu)-CO-(CH₂)₁₆-CO₂H)AQ-Aib- | | | |
| | | | EFIK(GGGGSGGGGSGAPAPAPAPAPAPG-BEA-MalDap₂-Fc)-αMeY- | | | |
| | | | LIEGSPSSGAPPPSGS-NH₂ | | | |
| 103 | | 56 | Y-Aib-EGT-αMeF(2F)-TSD-4Pal-Aib-I-αMeL-LD-Orn-K(ϵKϵK-(γGlu)-CO- | 30 | 217 | 935 |
| | | | (CH₂)₁₆-CO₂H)AQ-Aib-EFIE-αMeY-LIEGGPSSGAPPPSGK(PEG₂₄-BEA- | | | |
| | | | MalDap₂-Fc)-NH₂ | | | |
| 104 | | 562 | Y-Aib-EGT-αMeF(2F)-TSD-4Pal-Aib-I-αMeL-LD-Orn-K(ϵKϵK-(γGlu)-CO- | 31 | 218 | 935 |
| | | | (CH₂)₁₆-CO₂H)AQ-Aib-EFIE-αMeY-LIEGGESSGAPPPSGK(PEG₂₄-BEA- | | | |
| | | | MalDap₂-Fc)-NH₂ | | | |
| 105 | | 563 | Y-Aib-EGT-αMeF(2F)-TSD-4Pal-Aib-I-αMeL-LD-Orn-K(ϵKϵK-(γGlu)-CO- | 32 | 219 | 935 |
| | | | (CH₂)₁₆-CO₂H)AQ-Aib-EFIE-αMeY-LIEGG-Orn-SSGAPPPSGK(PEG₂₄- | | | |
| | | | BEA-MalDap₂-Fc)-NH₂ | | | |
| 106 | | 564 | Y-Aib-EGT-αMeF(2F)-TSD-4Pal-Aib-I-αMeL-LD-Orn-K(ϵKϵK-(γGlu)-CO- | 33 | 220 | 935 |
| | | | (CH₂)₁₆-CO₂H)AQ-Aib-EFIE-αMeY-LIEGGPPSGAPPPSGK(PEG₂₄-BEA- | | | |
| | | | MalDap₂-Fc)-NH₂ | | | |
| 107 | | 565 | Y-Aib-EGT-αMeF(2F)-TSD-4Pal-Aib-I-αMeL-LD-Orn-K(ϵKϵK-(γGlu)-CO- | 34 | 221 | 935 |
| | | | (CH₂)₁₆-CO₂H)AQ-Aib-EFIE-αMeY-LIEGGPPS-Aib-Orn-PPP-Orn- | | | |
| | | | GK(PEG₂₄-BEA-MalDap₂-Fc)-NH₂ | | | |
| 108 | | 566 | Y-Aib-EGT-αMeF(2F)-TSD-4Pal-Aib-I-αMeL-LD-Orn-K(-(γGlu)-CO- | 35 | 222 | 935 |
| | | | (CH₂)₁₆-CO₂H)AQ-Aib-EFIE-αMeY-LIEGSPSSGAPPPGGK(PEG₂₄-BEA- | | | |
| | | | MalDap₂-Fc)-NH₂ | | | |
| 109 | | 567 | Y-Aib-EGT-αMeF(2F)-TSD-4Pal-Aib-I-αMeL-LD-Orn-K(ϵKϵK-(γGlu)-CO- | 36 | 223 | 935 |
| | | | (CH₂)₁₆-CO₂H)AQ-Aib-EFIK(PEG₂₄-BEA-MalDap₂-Fc)-αMeY- | | | |
| | | | LIEGGPSSGAPPPSGG-NH₂ | | | |
| 110 | | 568 | Y-Aib-EGT-αMeF(2F)-TSD-4Pal-Aib-I-αMeL-LD-Orn-K(ϵKϵK-(γGlu)-CO- | 37 | 224 | 935 |
| | | | (CH₂)₁₆-CO₂H)AQ-Aib-EFIK(PEG₂₄-BEA-MalDap₂-Fc)-αMeY- | | | |
| | | | LIEGGESSGAPPPSGG-NH₂ | | | |
| 111 | | 569 | Y-Aib-EGT-αMeF(2F)-TSD-4Pal-Aib-I-αMeL-LD-Orn-K(ϵKϵK-(γGlu)-CO- | 38 | 225 | 935 |
| | | | (CH₂)₁₆-CO₂H)AQ-Aib-EFIK(PEG₂₄-BEA-MalDap₂-Fc)-αMeY-LIEGG-Orn- | | | |
| | | | SSGAPPPSGG-NH₂ | | | |
| 112 | | 570 | Y-Aib-EGT-αMeF(2F)-TSD-4Pal-Aib-I-αMeL-LD-Orn-K(ϵKϵK-(γGlu)-CO- | 39 | 226 | 935 |
| | | | (CH₂)₁₆-CO₂H)AQ-Aib-EFIK(PEG₂₄-BEA-MalDap₂-Fc)-αMeY- | | | |
| | | | LIEGGPPSGAPPPSGG-NH₂ | | | |
| 113 | | 571 | Y-Aib-EGT-αMeF(2F)-TSD-4Pal-Aib-I-αMeL-LD-Orn-K(ϵKϵK-(γGlu)-CO- | 40 | 227 | 935 |
| | | | (CH₂)₁₆-CO₂H)AQ-Aib-EFIK(PEG₂₄-BEA-MalDap₂-Fc)-αMeY-LIEGGPPS- | | | |
| | | | Aib-Orn-PPP-Orn-GG-NH₂ | | | |
| 114 | | 572 | Y-Aib-EGT-αMeF(2F)-TSD-4Pal-Aib-I-αMeL-LD-Orn-K(ϵKϵK-(γGlu)-CO- | 41 | 228 | 935 |
| | | | (CH₂)₁₆-CO₂H)AQ-Aib-EFIK(PEG₂₄-BEA-MalDap₂-Fc)-αMeY- | | | |
| | | | LIEGSPSSGAPPPGGG-NH₂ | | | |
| 115 | | 573 | Y-Aib-EGT-αMeF(2F)-TSD-4Pal-Aib-I-αMeL-LD-Orn-K(ϵKϵK-(γGlu)-CO- | 22 | 229 | 935 |
| | | | (CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 116 | | 574 | Y-Aib-EGT-αMeF(2F)-TSD-4Pal-Aib-I-αMeL-LD-Orn-K(ϵKϵK-(γGlu)-CO- | 42 | 230 | 935 |
| | | | (CH₂)₁₆-CO₂H)AQ-Aib-EFIE-αMeY-LIEGGPSSGK(BEA-MalDap₂-Fc)-NH₂ | | | |
| 117 | | 575 | Y-Aib-EGT-αMeF(2F)-TSD-4Pal-Aib-I-αMeL-LD-Orn-K(ϵKϵK-(γGlu)-CO- | 22 | 229 | 935 |
| | | | (CH₂)₁₆-CO₂H)AQ-Aib-EFIE-αMeY-LIEGGG-PEG₂₄-K(BEA-MalDap₂-Fc)- | | | |
| | | | NH₂ | | | |
| 118 | | 576 | Y-Aib-EGT-αMeF(2F)-TSD-4Pal-Aib-I-αMeL-LD-Orn-K(ϵKϵK-(γGlu)-CO- | 22 | 229 | 935 |
| | | | (CH₂)₁₆-CO₂H)AQ-Aib-EFIE-αMeY-LIEGGGK(PEG₂₄-BEA-MalDap₂-Fc)- | | | |
| | | | NH₂ | | | |
| 119 | | 577 | Y-Aib-EGT-αMeF(2F)-TSD-4Pal-Aib-I-αMeL-LD-Orn-K(ϵKϵK-(γGlu)-CO- | 43 | 231 | 935 |
| | | | (CH₂)₁₆-CO₂H)AQ-Aib-EFIK(PEG₂₄-BEA-MalDap₂-Fc)-αMeY-LIEGGG- | | | |
| | | | NH₂ | | | |
| 120 | | 578 | Y-Aib-EGT-αMeF(2F)-TSD-4Pal-Aib-I-αMeL-LD-Orn-K(ϵKϵK-(γGlu)-CO- | 44 | 232 | 935 |
| | | | (CH₂)₁₆-CO₂H)AQ-Aib-EFIK(PEG₂₄-BEA-MalDap₂-Fc)-αMeY- | | | |
| | | | LIEGGPSSG-NH₂ | | | |
| 121 | | 579 | Y-Aib-EGT-αMeF(2F)-TSD-4Pal-Aib-I-αMeL-LD-Orn-K(ϵKϵK-(γGlu)-CO- | 45 | 233 | 935 |
| | | | (CH₂)₁₆-CO₂H)AQ-Aib-EFIE-αMeY-LIK(PEG₂₄-BEA-MalDap₂-Fc)GGG- | | | |
| | | | NH₂ | | | |
| 122 | | 580 | Y-Aib-EGT-αMeF(2F)-TSD-4Pal-Aib-I-αMeL-LD-Orn-K(ϵKϵK(γGlu)-CO- | 46 | 234 | 935 |
| | | | (CH₂)₁₆-CO₂H)AQ-Aib-EFIE-αMeY-LIK(PEG₂₄-BEA-MalDap₂- | | | |
| | | | Fc)GGPSSG-NH₂ | | | |
| 123 | | 581 | Y-Aib-EGT-αMeF(2F)-TSD-4Pal-Aib-I-αMeL-LD-Orn-K(ϵKϵK-(γGlu)-CO- | 22 | 229 | 935 |
| | | | (CH₂)₁₆-CO₂H)AQ-Aib-EFIE-αMeY- | | | |
| | | | LIEGGGGGGGSGGGGSGAPAPAPAPAPAPK(BEA-MalDap₂-Fc)-NH₂ | | | |
| 124 | | 582 | Y-Aib-EGT-αMeF(2F)-TSD-4Pal-Aib-I-αMeL-LD-Orn-K(ϵKϵK-(γGlu)-CO- | 22 | 229 | 935 |
| | | | (CH₂)₁₆-CO₂H)AQ-Aib-EFIE-αMeY- | | | |
| | | | LIEGGGGGGGQGGGGQGGGGQK(BEA-MalDap₂-Fc)-NH₂ | | | |
| 125 | | 583 | Y-Aib-EGT-αMeF(2F)-TSD-4Pal-Aib-I-αMeL-LD-Orn-K(ϵKϵK-(γGlu)-CO- | 22 | 229 | 935 |
| | | | (CH₂)₁₆-CO₂H)AQ-Aib-EFIE-αMeY-LIEGGGGGGG-4Pal-GGGG-4Pal- | | | |
| | | | GGGG-4Pal-K(BEA-MalDap₂-Fc)-NH₂ | | | |
| 126 | | 584 | Y-Aib-EGT-αMeF(2F)-TSD-4Pal-Aib-I-αMeL-LD-Orn-K(ϵKϵK-(γGlu)-CO- | 42 | 230 | 935 |
| | | | (CH₂)₁₆-CO₂H)AQ-Aib-EFIE-αMeY-LIEGGPSSGK(PEG₂₄-BEA-MalDap₂- | | | |
| | | | Fc)-NH₂ | | | |
| 127 | 5 | 585 | Y-Aib-EGT-αMeF(2F)-TSD-4Pal-Aib-I-αMeL-LD-Orn-K(2-[2-(2-amino- | 43 | 235 | 935 |
| | | | ethoxy)-ethoxy]-acetyl)₂-(γGlu)-CO-(CH₂)₁₆-CO₂H)AQ-Aib-EFIK(PEG₂₄- | | | |
| | | | BEA-MalDap₂-Fc)-αMeY-LIEGGG-NH₂ | | | |
| 128 | | 586 | Y-Aib-EGT-αMeF(2F)-TSD-4Pal-Aib-I-αMeL-LD-Orn-K(2-[2-(2-amino- | 44 | 236 | 935 |
| | | | ethoxy)-ethoxy]-acetyl)₂-(γGlu)-CO-(CH₂)₁₆-CO₂H)AQ-Aib-EFIK(PEG₂₄- | | | |
| | | | BEA-MalDap₂-Fc)-αMeY-LIEGGPSSG-NH₂ | | | |
| 129 | 6 | 587 | Y-Aib-EGT-αMeF(2F)-TSD-4Pal-Aib-I-αMeL-LD-Orn-K(2-[2-(2-amino- | 45 | 237 | 935 |
| | | | ethoxy)-ethoxy]-acetyl)₂-(γGlu)-CO-(CH₂)₁₆-CO₂H)AQ-Aib-EFIE-αMeY- | | | |
| | | | LIK(PEG₂₄-BEA-MalDap₂-Fc)GGG-NH₂ | | | |
| 130 | | 588 | Y-Aib-EGT-αMeF(2F)-TSD-4Pal-Aib-I-αMeL-LD-Orn-K(2-[2-(2-amino- | 46 | 238 | 935 |
| | | | ethoxy)-ethoxy]-acetyl)₂-(γGlu)-CO-(CH₂)₁₆-CO₂H)AQ-Aib-EFIE-αMeY- | | | |
| | | | LIK(PEG₂₄-BEA-MalDap₂-Fc)GGPSSG-NH₂ | | | |
| 131 | | 589 | Y-Aib-EGT-αMeF(2F)-TSD-4Pal-Aib-I-αMeL-LD-Orn-K(2-[2-(2-amino- | 42 | 239 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGPSSGK(PEG₂₄-BEA-MalDap₂-Fc)-NH₂ | | | |
| 132 | | 590 | Y-Aib-EGT-αMeF(2F)-TSD-4Pal-Aib-I-αMeL-LD-Orn-K(2-[2-(2-amino- | 22 | 209 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGG-PEG₂₄-K(BEA-MalDap₂-Fc)-NH₂ | | | |
| 133 | | 591 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-MalDap₂-Fc)-NH₂ | | | |
| 134 | | 592 | Y-Aib-EGT-αMeF(2F)-TSD-4Pal-Aib-I-αMeL-LD-Orn-K(ϵKϵK-(γGlu)-CO- | 47 | 241 | 935 |
| | | | (CH₂)₁₆-CO₂H)AQ-Aib-EFIE-αMeY-LIEGSPSSGAPPPSK(PEG₂₄-BEA- | | | |
| | | | MalDap₂-Fc)-NH₂ | | | |
| 135 | | 593 | Y-Aib-EGT-αMeF(2F)-TSD-4Pal-Aib-I-αMeL-LDEK(ϵKϵK-(γGlu)-CO- | 5 | 242 | 935 |
| | | | (CH₂)₁₆-CO₂H)AQ-Aib-EFIE-αMeY-LIEGSPSSGAPPPSK(PEG₂₄-BEA- | | | |
| | | | MalDap₂-Fc)-NH₂ | | | |
| 136 | 7 | 594 | Y-Aib-EGT-αMeF(2F)-TSD-4Pal-Aib-I-αMeL-LD-Orn-K(2-[2-(2-amino- | 48 | 243 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGPSSGAPPPSK(PEG₂₄-BEA-MalDap₂-Fc)-NH₂ | | | |
| 137 | | 595 | Y-Aib-EGT-αMeF(2F)-TSD-4Pal-Aib-I-αMeL-LD-Orn-K(ϵKϵK-(γGlu)-CO- | 48 | 244 | 935 |
| | | | (CH₂)₁₆-CO₂H)AQ-Aib-EFIE-αMeY-LIEGGPSSGAPPPSK(PEG₂₄-BEA- | | | |
| | | | MalDap₂-Fc)-NH₂ | | | |
| 138 | | 596 | Y-Aib-EGT-αMeF(2F)-TSD-4Pal-Aib-I-αMeL-LDEK(ϵKϵK-(γGlu)-CO- | 49 | 245 | 935 |
| | | | (CH₂)₁₆-CO₂H)AQ-Aib-EFIE-αMeY-LIEGGPSSGAPPPSK(PEG₂₄-BEA- | | | |
| | | | MalDap₂-Fc)-NH₂ | | | |
| 139 | | 597 | Y-Aib-EGT-αMeF(2F)-TSD-4Pal-Aib-I-αMeL-LD-Orn-K(ϵKϵK-(γGlu)-CO- | 50 | 246 | 935 |
| | | | (CH₂)₁₆-C_{O2}H)AQ-Aib-EFIe-αMeY-LIEGSPSSGAPPPSK(PEG₂₄-BEA- | | | |
| | | | MalDap₂ Fc)-NH₂ | | | |
| 140 | | 598 | Y-Aib-EGT-αMeF(2F)-TSD-4Pal-Aib-I-αMeL-LD-Orn-K(ϵKϵK-(γGlu)-CO- | 27 | 247 | 935 |
| | | | (CH₂)₁₆-CO₂H)AQ-Aib-EFIe-αMeY-LIEGGGK(PEG₂₄-BEA-MalDap₂ Fc)- | | | |
| | | | NH₂ | | | |
| 141 | | 599 | Y-Aib-EGT-αMeF(2F)-TSD-4Pal-Aib-I-αMeL-LD-Orn-K(ϵKϵK-(γGlu)-CO- | 51 | 248 | 935 |
| | | | (CH₂)₁₆-CO₂H)AQ-Aib-EFIE-αMeY-LIEGG-Orn-SSGAPPPSK(PEG₂₄-BEA- | | | |
| | | | MalDap₂ Fc)-NH₂ | | | |
| 142 | | 600 | Y-Aib-EGT-aMcF(2F)-TSD-4Pal-Aib-I-αMeL-LD-Orn-K(2-[2-(2-amino- | 51 | 249 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGG-Orn-SSGAPPPSK(PEG₂₄-BEA-MalDap₂ Fc)-NH2 | | | |
| 143 | | 601 | Y-Aib-EGT-αMeF(2F)-TSD-4Pal-Aib-I-αMeL-LD-Orn-K(ϵKϵK-(γGlu)-CO- | 52 | 250 | 935 |
| | | | (CH₂)₁₆-CO₂H)AQ-Aib-EFIE-αMeY-LIEGG-Orn-K(PEG₂₄-BEA-MalDap₂ | | | |
| | | | Fc)-NH₂ | | | |
| 144 | | 602 | Y-Aib-EGT-αMeF(2F)-TSD-4Pal-Aib-I-αMeL-LD-Orn-K(2-12-(2-amino- | 52 | 251 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGG-Orn-K(PEG₂₄-BEA-MalDap₂ Fc)-NH₂ | | | |
| 145 | | 603 | Y-Aib-EGT-αMeF(2F)-TSD-4Pal-Aib-I-αMeL-LD-Orn-K(ϵKϵK-(γGlu)-CO- | 53 | 252 | 935 |
| | | | (CH₂)₁₆-CO₂H)AQ-Aib-Orn-FIE-αMeY-LIEGSPSSGAPPPSK(PEG₂₄-BEA- | | | |
| | | | MalDap₂ Fc)-NH₂ | | | |
| 146 | | 604 | Y-Aib-EGT-αMeF(2F)-TSD-4Pal-Aib-I-αMeL-LD-Orn-K(2-[2-(2-amino- | 53 | 253 | 935 |
| | | | ethoxy)-ethoxy]-acetyl)₂-(γGlu)-CO-(CH₂)₁₆-CO₂H)AQ-Aib-Orn-FIE-αMeY- | | | |
| | | | LIEGSPSSGAPPPSK(PEG₂₄-BEA-MalDap₂ Fc)-NH₂ | | | |
| 147 | | 605 | Y-Aib-EGT-αMeF(2F)-TSD-4Pal-Aib-I-αMeL-LD-Orn-K(2-[2-(2-amino- | 54 | 254 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂ Fc)-NH₂ | | | |
| 148 | | 606 | Y-Aib-EGT-αMeF(2F)-TSD-4Pal-Aib-I-αMeL-LD-Orn-K(ϵKϵK-(γGlu)-CO- | 54 | 255 | 935 |
| | | | (CH₂)₁₆-CO₂H)AQ-Aib-EFIQ-αMeY-LIEGSPSSGAPPPSK(PEG₂₄-BEA- | | | |
| | | | MalDap₂ Fc)-NH₂ | | | |
| 149 | | 607 | Y-Aib-EGT-αMeF(2F)-TSD-4Pal-Aib-I-αMeL-LD-Orn-K(ϵKϵK-(γGlu)-CO- | 47 | 241 | 935 |
| | | | (CH₂)₁₆-CO₂H)AQ-Aib-EFIE-αMeY-LIEGSPSSGAPPPSK(2-[2-(2-amino- | | | |
| | | | ethoxy)-ethoxy]-acetyl)₆-GPAPAPAPAPAPA-BEA-MalDap₂-Fc)-NH₂ | | | |
| 150 | | 608 | Y-Aib-EGT-αMeF(2F)-TSD-4Pal-Aib-I-αMeL-LD-Orn-K(&K&K-(γGlu)-CO- | 50 | 246 | 935 |
| | | | (CH₂)₁₆-CO₂H)AQ-Aib-EFIe-αMeY-LIEGSPSSGAPPPSK(2-[2-(2-amino- | | | |
| | | | ethoxy)-ethoxy]-acetyl),-GPAPAPAPAPAPA-BEA-MalDap₂-Fc)-NH₂ | | | |
| 151 | | 609 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄- | | | |
| | | | GPAPAPAPAPAPA-BEA-MalDap₂-Fc)-NH₂ | | | |
| 152 | | 610 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄-GPAPAPAPA- | | | |
| | | | BEA-MalDap₂-Fc)-NH₂ | | | |
| 153 | | 611 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄-GPAPA-BEA- | | | |
| | | | MalDap₂-Fc)-NH₂ | | | |
| 154 | | 612 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc)-NH₂ | | | |
| 155 | | 613 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆-GPAPAPAPA- | | | |
| | | | BEA-MalDap₂-Fc)-NH₂ | | | |
| 156 | | 614 | Y-Aib-EGT-αMeF(2F)-TSD-4Pal-Aib-I-αMeL-LD-Orn-K(2-12-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆-GPAPA-BEA- | | | |
| | | | MalDap₂-Fc)-NH₂ | | | |
| 157 | | 615 | Y-Aib-EGT-αMeF(2F)-TSD-4Pal-SI-αMeL-LD-Orn-K(2-[2-(2-amino- | 55 | 256 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc)-NH₂ | | | |
| 158 | 8 | 616 | Y-Aib-EGT-αMeF(2F)-TSD-4Pal-SI-αMeL-LD-Orn-K(ϵKϵK-(γGlu)-CO- | 55 | 257 | 935 |
| | | | (CH₂)₁₆-CO₂H)AQ-Aib-EFIE-αMeY-LIEGSPSSGAPPPSK(2-[2-(2-amino- | | | |
| | | | ethoxy)-ethoxy]-acetyl)₆-GPAPAPAPAPAPA-BEA-MalDap₂-Fc)-NH₂ | | | |
| 159 | | 617 | Y-Aib-EGT-αMeF(2F)-TSD-4Pal-SI-αMeL-LD-Orn-K(ϵKϵK-(γGlu)-CO- | 2 | 258 | 935 |
| | | | (CH2)16-CO2H)AQ-Aib-EFIe-αMeY-LIEGSPSSGAPPPSK(2-[2-(2-amino- | | | |
| | | | ethoxy)-ethoxy]-acetyl)₆-GPAPAPAPAPAPA-BEA-MalDap₂-Fc)-NH₂ | | | |
| 160 | | 618 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄-BEA- | | | |
| | | | MalDap₂-Fc)-NH₂ | | | |
| 161 | | 619 | Y-Aib-EGT-αMeF(2F)-TSD-4Pal-Aib-I-αMeL-LD-Orn-K(2-12-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 162 | | 620 | Y-Aib-EGT-αMeF(2F)-TSD-4Pal-Aib-I-αMeL-LD-Orn-K(2-[2-(2-amino- | 47 | 240 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₄-BEA- | | | |
| | | | MalDap₂-Fc)-NH₂ | | | |
| 163 | | 621 | Y-Aib-EGT-αMeF(2F)-TSD-4Pal-Aib-I-αMeL-LD-Orn-K(2-[2-(2-amino- | 56 | 259 | 935 |
| | | | ethoxy)-ethoxy]-acetyl)₂-(γGlu)-CO-(CH₂)₁₆-CO₂H)AQ-Aib-EFIE-αMeY- | | | |
| | | | LIE-Aib-SPSSGAPPPSK(PEG₂₄-BEA-MalDap₂-Fc)-NH₂ | | | |
| 164 | | 622 | Y-Aib-EGT-αMeF(2F)-TSD-4Pal-Aib-I-αMeL-LD-Orn-K(ϵKϵK-(γGlu)-CO- | 56 | 260 | 935 |
| | | | (CH₂)₁₆-CO₂H)AQ-Aib-EFIE-αMeY-LIE-Aib-SPSSGAPPPSK(PEG₂₄-BEA- | | | |
| | | | MalDap₂-Fc)-NH₂ | | | |
| 165 | | 623 | Y-Aib-EGT-αMeF(2F)-TSD-4Pal-Aib-I-αMeL-LD-Orn-K(ϵKϵK-(γGlu)-CO- | 57 | 261 | 935 |
| | | | (CH₂)₁₆-CO₂H)AQ-Aib-EFIe-αMeY-LIE-Aib-SPSSGAPPPSK(PEG₂₄-BEA- | | | |
| | | | MalDap₂-Fc)-NH₂ | | | |
| 166 | | 624 | Y-Aib-EGT-αMeF(2F)-TSD-4Pal-SI-αMeL-LD-Orn-K(2-[2-(2-amino- | 55 | 256 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-MalDap₂-Fc)-NH₂ | | | |
| 167 | | 625 | Y-Aib-EGT-αMeF(2F)-TSD-4Pal-SI-αMeL-LD-Orn-K(2-[2-(2-amino- | 58 | 262 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂-Fc)-NH₂ | | | |
| 168 | 9 | 626 | Y-Aib-EGT-αMeF(2F)-TSD-4Pal-SI-αMeL-LD-Orn-K(ϵKϵK-(γGlu)-CO- | 55 | 257 | 935 |
| | | | (CH₂)₁₆-CO₂H)AQ-Aib-EFIE-αMeY-LIEGSPSSGAPPPSK(PEG₂₄-BEA- | | | |
| | | | MalDap₂-Fc)-NH₂ | | | |
| 169 | 10 | 627 | Y-Aib-EGT-aMcF(2F)-TSD-4Pal-SI-αMeL-LD-Orn-K(ϵKϵK-(γGlu)-CO- | 58 | 263 | 935 |
| | | | (CH₂)₁₆-CO₂H)AQ-Aib-EFIQ-αMeY-LIEGSPSSGAPPPSK(PEG₂₄-BEA- | | | |
| | | | MalDap₂-Fc)-NH₂ | | | |
| 170 | 11 | 628 | Y-Aib-EGT-aMcF(2F)-TSDY-Aib-I-αMeL-LD-Orn-K(2-[2-(2-amino- | 59 | 264 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-MalDap₂-Fc)-NH₂ | | | |
| 171 | | 629 | Y-Aib-EGT-αMeF(2F)-TSDY-Aib-I-αMeL-LD-Orn-K(2-[2-(2-amino- | 60 | 265 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂-Fc)-NH₂ | | | |
| 172 | 12 | 630 | Y-Aib-EGT-αMeF(2F)-TSDY-Aib-I-αMeL-LD-Orn-K(ϵKϵK-(γGlu)-CO- | 61 | 266 | 935 |
| | | | (CH₂)₁₆-CO₂H)AQ-Aib-EFIe-αMeY-LIEGSPSSGAPPPSK(PEG₂₄-BEA- | | | |
| | | | MalDap₂-Fc)-NH₂ | | | |
| 173 | 13 | 631 | Y-Aib-EGT-αMeF(2F)-TSDY-Aib-I-αMeL-LD-Orn-K(ϵKϵK-(γGlu)-CO- | 59 | 267 | 935 |
| | | | (CH₂)₁₆-CO₂H)AQ-Aib-EFIE-αMeY-LIEGSPSSGAPPPSK(PEG₂₄-BEA- | | | |
| | | | MalDap₂-Fc)-NH₂ | | | |
| 174 | | 632 | Y-Aib-EGT-αMeF(2F)-TSDY-Aib-I-αMeL-LD-Orn-K(ϵKϵK-(γGlu)-CO- | 60 | 268 | 935 |
| | | | (CH₂)₁₆-CO₂H)AQ-Aib-EFIQ-αMeY-LIEGSPSSGAPPPSK(PEG₂₄-BEA- | | | |
| | | | MalDap₂-Fc)-NH₂ | | | |
| 175 | | 633 | Y-Aib-EGT-αMeF(2F)-TSD-4Pal-Aib-I-αMeL-LD-Orn-K(ϵKϵK-(γGlu)-CO- | 62 | 269 | 935 |
| | | | (CH₂)₁₆-CO₂H)AQ-αMeL-EFIE-αMeY-LIEGSPSSGAPPPSK(PEG₂₄-BEA- | | | |
| | | | MalDap₂-Fc)-NH₂ | | | |
| 176 | | 634 | Y-Aib-EGT-αMeF(2F)-TSD-4Pal-Aib-I-αMeL-LD-Orn-K(ϵKϵK-(γGlu)-CO- | 63 | 270 | 935 |
| | | | (CH₂)₁₆-CO₂H)AQ-Iva-EFIE-αMeY-LIEGSPSSGAPPPSK(PEG₂₄-BEA- | | | |
| | | | MalDap₂-Fc)-NH₂ | | | |
| 177 | | 635 | Y-Aib-EGT-αMeF(2F)-TSD-4Pal-Aib-I-αMeL-LD-Orn-K(2-[2-(2-amino- | 64 | 271 | 935 |
| | | | ethoxy)-ethoxy]-acetyl)₂-(γGlu)-CO-(CH₂)₁₆-CO₂H)AQ-αMeL-EFIQ-αMeY- | | | |
| | | | LIEGSPSSGAPPPSK(PEG₂₄-BEA-MalDap₂-Fc)-NH₂ | | | |
| 178 | | 636 | Y-Aib-EGT-αMeF(2F)-TSD-4Pal-Aib-I-αMeL-LD-Orn-K(2-[2-(2-amino- | 65 | 272 | 935 |
| | | | ethoxy)-ethoxy]-acetyl)₂-(γGlu)-CO-(CH₂)₁₆-CO₂H)AQ-Iva-EFIQ-αMeY- | | | |
| | | | LIEGSPSSGAPPPSK(PEG₂₄-BEA-MalDap₂-Fc)-NH₂ | | | |
| 179 | | 637 | Y-Aib-EGT-αMeF(2F)-TSD-4Pal-Aib-I-αMeL-LD-Orn-K(ϵKϵK-(γGlu)-CO- | 64 | 273 | 935 |
| | | | (CH₂)₁₆-CO₂H)AQ-_{α}MeL-EFIQ-αMeY-LIEGSPSSGAPPPSK(PEG₂₄-BEA- | | | |
| | | | MalDap₂-Fc)-NH₂ | | | |
| 180 | | 638 | Y-Aib-EGT-αMeF(2F)-TSD-4Pal-Aib-I-αMeL-LD-Orn-K(ϵKϵK-(γGlu)-CO- | 65 | 274 | 935 |
| | | | (CH₂)₁₆-CO₂H)AQ-Iva-EFIQ-αMeY-LIEGSPSSGAPPPSK(PEG₂₄-BEA- | | | |
| | | | MalDap₂-Fc)-NH₂ | | | |
| 181 | 14 | 639 | Y-Aib-EGT-αMeF(2F)-TSD-4Pal-Aib-I-αMeL-LD-Orn-K(2-[2-(2-amino- | 66 | 275 | 935 |
| | | | ethoxy)-ethoxy]-acetyl)₂-(γGlu)-CO-(CH₂)₁₆-CO₂H)AQ-Aib-QFIE-αMeY- | | | |
| | | | LIEGSPSSGAPPPSK(PEG₂₄-BEA-MalDap₂-Fc)-NH₂ | | | |
| 182 | | 640 | Y-Aib-EGT-αMeF(2F)-TSD-4Pal-Aib-I-αMeL-LD-Orn-K(ϵKϵK-(γGlu)-CO- | 67 | 276 | 935 |
| | | | (CH₂)₁₆-CO₂H)AQ-Aib-EFIQ-αMeY-LLEGSPSSGAPPPSK(PEG₂₄-BEA- | | | |
| | | | MalDap₂-Fc)-NH₂ | | | |
| 183 | 15 | 641 | Y-Aib-EGT-αMeF(2F)-TSD-4Pal-SI-αMeL-LD-Orn-K(2-[2-(2-amino- | 58 | 262 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc)-NH₂ | | | |
| 184 | 16 | 642 | Y-Aib-EGT-αMeF(2F)-TSD-4Pal-SI-αMeL-LD-Orn-K(2-[2-(2-amino- | 58 | 262 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 185 | 17 | 643 | Y-Aib-EGT-αMeF(2F)-TSD-4Pal-SI-αMeL-LD-Orn-K(ϵKϵK-(γGlu)-CO- | 58 | 263 | 935 |
| | | | (CH2)16-CO2H)AQ-Aib-EFIQ-αMeY-LIEGSPSSGAPPPSK(2-[2-(2-amino- | | | |
| | | | ethoxy)-ethoxy]-acetyl)6-BEA-MalDap2-Fc)-NH2 | | | |
| 186 | | 644 | Y-Aib-EGT-αMeF(2F)-TSD-4Pal-SI-αMeL-LD-Orn-K(2-[2-(2-amino- | 21 | 208 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc)-NH₂ | | | |
| 187 | 18 | 645 | Y-Aib-EGT-αMeF(2F)-TSD-4Pal-SI-αMeL-LD-Orn-K(2-[2-(2-amino- | 68 | 277 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGGGGGGGSGGGGSGGGGSK(BEA-MalDap₂-Fc)-NH₂ | | | |
| 188 | 19 | 646 | Y-Aib-EGT-αMeF(2F)-TSD-4Pal-SI-αMeL-LD-Orn-K(2-[2-(2-amino- | 68 | 277 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGGGGGGGSGGGGSGAPAPAPAPAPAPK(BEA-MalDap₂-Fc)-NH₂ | | | |
| 189 | | 647 | Y-Aib-EGT-αMeF(2F)-TSD-4Pal-SI-αMeL-LD-Orn-K(ϵKϵK-(γGlu)-CO- | 21 | 278 | 935 |
| | | | (CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 190 | | 648 | Y-Aib-EGT-αMeF(2F)-TSD-4Pal-SI-αMeL-LD-Orn-K(ϵKϵK-(γGlu)-CO- | 68 | 279 | 935 |
| | | | (CH₂)₁₆-CO₂H)AQ-Aib-EFIQ-αMeY- | | | |
| | | | LIEGGGGGGGSGGGGSGGGGSK(BEA-MalDap₂-Fc)-NH₂ | | | |
| 191 | | 649 | Y-Aib-EGT-αMeF(2F)-TSD-4Pal-SI-αMeL-LD-Orn-K(ϵKϵK-(γGlu)-CO- | 68 | 279 | 935 |
| | | | (CH₂)₁₆-CO₂H)AQ-Aib-EFIQ-αMeY- | | | |
| | | | LIEGGGGGGGSGGGGSGAPAPAPAPAPAPK(BEA-MalDap2-Fc)-NH₂ | | | |
| 192 | | 650 | Y-Aib-EGT-αMeF(2F)-TSD-4Pal-Aib-I-αMeL-LD-Orn-K(2-[2-(2-amino- | 54 | 254 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc)-NH₂ | | | |
| 193 | | 651 | Y-Aib-EGT-αMeF(2F)-TSD-4Pal-Aib-I-αMeL-LD-Orn-K(2-[2-(2-amino- | 54 | 254 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 194 | | 652 | Y-Aib-EGT-αMeF(2F)-TSD-4Pal-Aib-I-αMeL-LD-Orn-K(2-[2-(2-amino- | 22 | 209 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc)-NH₂ | | | |
| 195 | | 653 | Y-Aib-EGT-αMeF(2F)-TSD-4Pal-Aib-I-αMeL-LD-Orn-K(2-[2-(2-amino- | 69 | 280 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGGGGGGGSGGGGSGGGGSK(BEA-MalDap₂-Fc)-NH₂ | | | |
| 196 | | 654 | Y-Aib-EGT-αMeF(2F)-TSD-4Pal-Aib-I-αMeL-LD-Orn-K(2-[2-(2-amino- | 69 | 280 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGGGGGGGSGGGGSGAPAPAPAPAPAPK(BEA-MalDap₂-Fc)-NH₂ | | | |
| 197 | | 655 | Y-Aib-EGT-αMeF(2F)-TSD-4Pal-Aib-I-αMeL-LD-Orn-K(2-[2-(2-amino- | 70 | 281 | 935 |
| | | | ethoxy)-ethoxy]-acetyl)₂-(γGlu)-CO-(CH₂)₁₆-CO₂H)AQ-Aib-DFIQ-αMeY- | | | |
| | | | LIEGSPSSGAPPPSK(2-[2-(2-amino-ethoxy)-ethoxy]-acetyl)₆-BEA- | | | |
| | | | MalDap2-Fc)-NH2 | | | |
| 198 | | 656 | Y-Aib-EGT-αMeF(2F)-TSD-4Pal-SI-αMeL-LD-Orn-K(2-[2-(2-amino- | 71 | 282 | 935 |
| | | | ethoxy)-ethoxy]-acetyl)2-(γGlu)-CO-(CH2)16-CO2H)AQ-Aib-EFIK(PEG₂₄- | | | |
| | | | BEA-MalDap₂-Fc)-αMeY-LIEGSPSSGAPPPS-NH₂ | | | |
| 199 | | 657 | Y-Aib-EGT-αMeF(2F)-TSD-4Pal-Aib-I-αMeL-LD-Orn-K(2-[2-(2-amino- | 72 | 283 | 935 |
| | | | ethoxy)-ethoxy]-acetyl)₂-(γGlu)-CO-(CH₂)₁₆-CO₂H)AQ-Aib-EFIK(PEG₂₄- | | | |
| | | | BEA-MalDap₂-Fc)-αMeY-LIEGSPSSGAPPPS-NH₂ | | | |
| 200 | | 658 | Y-Aib-EGT-αMeF(2F)-TSD-4Pal-SI-αMeL-LD-Orn-K(2-[2-(2-amino- | 71 | 282 | 935 |
| | | | ethoxy)-ethoxy]-acetyl)₂-(γGlu)-CO-(CH₂)₁₆-CO₂H)AQ-Aib-EFIK(2-[2-(2- | | | |
| | | | amino-ethoxy)-ethoxy]-acetyl)(-BEA-MalDap₂-Fc)-αMeY- | | | |
| | | | LIEGSPSSGAPPPS-NH₂ | | | |
| 201 | | 659 | Y-Aib-EGT-αMeF(2F)-TSD-4Pal-Aib-I-αMeL-LD-Orn-K(2-[2-(2-amino- | 72 | 283 | 935 |
| | | | ethoxy)-ethoxy]-acetyl)₂-(γGlu)-CO-(CH₂)₁₆-CO₂H)AQ-Aib-EFIK(2-[2-(2- | | | |
| | | | amino-ethoxy)-ethoxy]-acetyl)(-BEA-MalDap₂-Fc)-αMeY- | | | |
| | | | LIEGSPSSGAPPPS-NH₂ | | | |
| 202 | | 660 | Y-Aib-EGT-αMeF(2F)-TSD-4Pal-Aib-I-αMeL-LD-Orn-K(eK-(2-[2-(2- | 54 | 284 | 935 |
| | | | amino-ethoxy)-ethoxy]-acetyl)-(γGlu)-CO-(CH₂)₆-CO₂H)AQ-Aib-EFIQ- | | | |
| | | | αMeY-LIEGSPSSGAPPPSK(2-[2-(2-amino-ethoxy)-ethoxy]-acetyl)₆-BEA- | | | |
| | | | MalDap₂-Fc)-NH₂ | | | |
| 203 | | 661 | Y-Aib-EGT-αMeF(2F)-TSD-4Pal-Aib-I-αMeL-LD-Orn-K(2-[2-(2-amino- | 54 | 285 | 935 |
| | | | ethoxy)-ethoxy]-acetyl)-eK-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 204 | 20 | 662 | Y-Aib-EGT-αMeF(2F)-TSD-4Pal-Aib-I-αMeL-LD-Orn-K(ϵKϵK-(γGlu)-CO- | 47 | 241 | 935 |
| | | | (CH₂)₁₆-CO2H)AQ-Aib-EFIE-αMeY-LIEGSPSSGAPPPSK(2-[2-(2-amino- | | | |
| | | | ethoxy)-ethoxy]-acetyl)₆-BEA-MalDap₂-Fc)-NH₂ | | | |
| 205 | | 663 | Y-Aib-EGT-αMeF(2F)-TSD-4Pal-SI-αMeL-LD-Orn-K(ϵKϵK-(γGlu)-CO- | 55 | 257 | 935 |
| | | | (CH₂)₁₆-CO₂H)AQ-Aib-EFIE-αMeY-LIEGSPSSGAPPPSK(2-[2-(2-amino- | | | |
| | | | ethoxy)-ethoxy]-acetyl)₆-BEA-MalDap₂-Fc)-NH₂ | | | |
| 206 | | 664 | Y-Aib-EGT-αMeF(2F)-TSDY-Aib-I-αMeL-LD-Orn-K(2-[2-(2-amino- | 59 | 264 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 207 | | 665 | Y-Aib-EGT-αMeF(2F)-TSDY-Aib-I-αMeL-LD-Orn-K(2-[2-(2-amino- | 60 | 265 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 208 | | 666 | Y-Aib-EGT-αMeF(2F)-TSDY-Aib-I-αMeL-LD-Orn-K(ϵKϵK-(γGlu)-CO- | 59 | 267 | 935 |
| | | | (CH₂)₁₆-CO₂H)AQ-Aib-EFIE-αMeY-LIEGSPSSGAPPPSK(2-[2-(2-amino- | | | |
| | | | ethoxy)-ethoxy]-acetyl)₆-BEA-MalDap₂-Fc)-NH₂ | | | |
| 209 | | 667 | Y-Aib-EGT-αMeF(2F)-TSDY-Aib-I-αMeL-LD-Orn-K(ϵKϵK-(γGlu)-CO- | 60 | 268 | 935 |
| | | | (CH₂)₁₆-CO₂H)AQ-Aib-EFIQ-αMeY-LIEGSPSSGAPPPSK(2-[2-(2-amino- | | | |
| | | | ethoxy)-ethoxy]-acetyl)₆-BEA-MalDap₂-Fc)-NH₂ | | | |
| 210 | | 668 | Y-Aib-EGT-αMeF(2F)-TSDYSI-αMeL-LD-Orn-K(2-[2-(2-amino-ethoxy)- | 73 | 286 | 935 |
| | | | ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 211 | | 669 | Y-Aib-EGT-αMeF(2F)-TSDYSI-αMeL-LD-Orn-K(2-[2-(2-amino-ethoxy)- | 74 | 287 | 935 |
| | | | ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆-BEA- | | | |
| | | | MalDap₂-Fc)-NH₂ | | | |
| 212 | | 670 | Y-Aib-EGT-αMeF(2F)-TSDYSI-αMeL-LD-Orn-K(ϵKϵK-(γGlu)-CO-(CH₂)₁₆- | 73 | 288 | 935 |
| | | | CO₂H)AQ-Aib-EFIE-αMeY-LIEGSPSSGAPPPSK(2-[2-(2-amino-ethoxy)- | | | |
| | | | ethoxy]-acetyl)₆-BEA-MalDap₂-Fc)-NH₂ | | | |
| 213 | | 671 | Y-Aib-EGT-αMeF(2F)-TSDYSI-αMeL-LD-Orn-K(ϵKϵK-(γGlu)-CO-(CH₂)₁₆- | 74 | 289 | 935 |
| | | | CO₂H)AQ-Aib-EFIQ-αMeY-LIEGSPSSGAPPPSK(2-[2-(2-amino-ethoxy)- | | | |
| | | | ethoxy]-acetyl)₆-BEA-MalDap₂-Fc)-NH₂ | | | |
| 214 | | 672 | Y-Aib-EGT-αMeF(2F)-TSD-4Pal-Aib-I-αMeL-LD-Orn-K(ϵKϵK-(γGlu)-CO- | 69 | 290 | 935 |
| | | | (CH₂)₁₆-CO2H)AQ-Aib-EFIQ-αMeY- | | | |
| | | | LIEGGGGGGGQGGGGQGGGGQK(BEA-MalDap₂-Fc)-NH₂ | | | |
| 215 | | 673 | Y-Aib-EGT-αMeF(2F)-TSD-4Pal-SI-αMeL-LD-Orn-K(ϵKϵK-(γGlu)-CO- | 68 | 279 | 935 |
| | | | (CH₂)₁₆-CO₂H)AQ-Aib-EFIQ-αMeY- | | | |
| | | | LIEGGGGGGGQGGGGQGGGGQK(BEA-MalDap₂-Fc)-NH₂ | | | |
| 216 | | 674 | Y-Aib-EGT-αMeF(2F)-TSDY-Aib-I-αMeL-LD-Orn-K(2-[2-(2-amino- | 75 | 292 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 217 | | 675 | H-Aib-EGT-αMeF(2F)-TSD-4Pal-SI-αMeL-LD-Orn-K(2-[2-(2-amino- | 76 | 293 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc)-NH₂ | | | |
| 218 | | 676 | H-Aib-EGT-αMeF(2F)-TSD-4Pal-SI-αMeL-LD-Orn-K(ϵKϵK-(γGlu)-CO- | 77 | 294 | 935 |
| | | | (CH₂)₁₆-CO₂H)AQ-Aib-EFIE-αMeY-LIEGSPSSGAPPPSK(PEG₂₄-BEA- | | | |
| | | | MalDap₂-Fc)-NH₂ | | | |
| 219 | | 677 | Y-Aib-EGT-αMeF(2F)-TSD-4Pal-Aib-I-αMeL-LD-Orn-K(2-12-(2-amino- | 78 | 295 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEQGGGGGGSGGGGSGGGGSK(BEA-MalDap₂-Fc)-NH₂ | | | |
| 220 | | 678 | Y-Aib-EGT-αMeF(2F)-TSD-4Pal-SI-αMeL-LD-Orn-K(ϵKϵK-(γGlu)-CO- | 79 | 296 | 935 |
| | | | (CH₂)₁₆-CO₂H)AQ-Aib-EFIe-αMeY-LIEGGPSSGAPPPSK(PEG₂₄-BEA- | | | |
| | | | MalDap₂-Fc)-NH₂ | | | |
| 221 | | 679 | Y-Aib-EGT-αMeF(2F)-TSD-4Pal-Aib-I-αMeL-LD-Orn-K(ϵKϵK-(γGlu)-CO- | 80 | 297 | 935 |
| | | | (CH₂)₁₆-CO₂H)AQ-Aib-EFIe-αMeY-LIEGGPSSGAPPPSK(PEG₂₄-BEA- | | | |
| | | | MalDap₂-Fc)-NH₂ | | | |
| 222 | | 680 | Y-Aib-EGT-αMeF(2F)-TSDY-Aib-I-αMeL-LD-Orn-K(2-[2-(2-amino- | 81 | 298 | 935 |
| | | | ethoxy)-ethoxy]-acetyl)₂-(γGlu)-CO-(CH₂)₁₆-CO₂H)AQ-Aib-EFIe-αMeY- | | | |
| | | | LIEGGPSSGAPPPSK(PEG₂₄-BEA-MalDap₂-Fc)-NH₂ | | | |
| 223 | | 681 | Y-Aib-EGT-αMeF(2F)-TSDY-Aib-I-αMeL-LD-Orn-K(ϵKϵK-(γGlu)-CO- | 81 | 299 | 935 |
| | | | (CH₂)₁₆-CO₂H)AQ-Aib-EFIe-αMeY-LIEGGPSSGAPPPSK(PEG₂₄-BEA- | | | |
| | | | MalDap₂-Fc)-NH₂ | | | |
| 224 | | 682 | Y-Aib-EGT-αMeF(2F)-TSD-4Pal-SI-αMeL-LD-Orn-K(2-[2-(2-amino- | 82 | 300 | 935 |
| | | | ethoxy)-ethoxy]-acetyl)₂-(γGlu)-CO-(CH₂)₁₆-CO₂H)AQ-Aib-EFIe-αMeY- | | | |
| | | | LIAGSPSSGAPPPSK(PEG₂₄-BEA-MalDap₂-Fc)-NH₂ | | | |
| 225 | | 683 | Y-Aib-EGT-αMeF(2F)-TSD-4Pal-SI-αMeL-LD-Orn-K(ϵKϵK-(γGlu)-CO- | 82 | 301 | 935 |
| | | | (CH₂)₁₆-CO₂H)AQ-Aib-EFIe-αMeY-LIAGSPSSGAPPPSK(PEG₂₄-BEA- | | | |
| | | | MalDap₂-Fc)-NH₂ | | | |
| 226 | | 684 | Y-Aib-EGT-aMcF(2F)-TSD-4Pal-Aib-I-αMeL-LD-Orn-K(ϵKϵK-(γGlu)-CO- | 83 | 302 | 935 |
| | | | (CH₂)₁₆-CO₂H)AQ-Aib-EFIe-αMeY-LIAGSPSSGAPPPSK(PEG₂₄-BEA- | | | |
| | | | MalDap₂-Fc)-NH₂ | | | |
| 227 | | 685 | Y-Aib-EGT-αMeF(2F)-TSDY-Aib-I-αMeL-LD-Orn-K(2-[2-(2-amino- | 84 | 303 | 935 |
| | | | ethoxy)-ethoxy]-acetyl)₂-(γGlu)-CO-(CH₂)₁₆-CO₂H)AQ-Aib-EFIe-αMeY- | | | |
| | | | LIAGSPSSGAPPPSK(PEG₂₄-BEA-MalDap₂-Fc)-NH₂ | | | |
| 228 | | 686 | Y-Aib-EGT-αMeF(2F)-TSDY-Aib-I-αMeL-LD-Orn-K(ϵKϵK-(γGlu)-CO- | 84 | 304 | 935 |
| | | | (CH₂)₁₆-CO₂H)AQ-Aib-EFIe-αMeY-LIAGSPSSGAPPPSK(PEG₂₄-BEA- | | | |
| | | | MalDap₂-Fc)-NH₂ | | | |
| 229 | | 687 | Y-Aib-EGT-αMeF(2F)-TSD-4Pal-SI-αMeL-LD-Orn-K(ϵKϵK-(γGlu)-CO- | 85 | 305 | 935 |
| | | | (CH₂)₁₆-CO₂H)AQ-Aib-EFIe-αMeY-LIAGGPSSGAPPPSK(PEG₂₄-BEA- | | | |
| | | | MalDap₂-Fc)-NH₂ | | | |
| 230 | | 688 | Y-Aib-EGT-αMeF(2F)-TSD-4Pal-SI-αMeL-LD-Orn-K(2-[2-(2-amino- | 85 | 306 | 935 |
| | | | ethoxy)-ethoxy]-acetyl)₂-(γGlu)-CO-(CH₂)₁₆-CO₂H)AQ-Aib-EFIe-αMeY- | | | |
| | | | LIAGGPSSGAPPPSK(PEG₂₄-BEA-MalDap₂-Fc)-NH₂ | | | |
| 231 | | 689 | Y-Aib-EGT-αMeF(2F)-TSDY-Aib-I-αMeL-LD-Orn-K(ϵKϵK-(γGlu)-CO- | 86 | 307 | 935 |
| | | | (CH₂)₁₆-CO₂H)AQ-Aib-EFIe-αMeY-LIAGGPSSGAPPPSK(PEG₂₄-BEA- | | | |
| | | | MalDap₂-Fc)-NH₂ | | | |
| 232 | | 690 | Y-Aib-EGT-αMeF(2F)-TSDY-Aib-I-αMeL-LD-Orn-K(2-[2-(2-amino- | 86 | 308 | 935 |
| | | | ethoxy)-ethoxy]-acetyl)₂-(γGlu)-CO-(CH₂)₁₆-CO₂H)AQ-Aib-EFIe-αMeY- | | | |
| | | | LIAGGPSSGAPPPSK(PEG₂₄-BEA-MalDap₂-Fc)-NH₂ | | | |
| 233 | | 69 | Y-Aib-EGT-αMeF(2F)-TSD-4Pal-SI-αMeL-LD-Orn-K(2-[2-(2-amino- | 2 | 309 | 935 |
| | | | ethoxy)-ethoxy]-acetyl)-(γGlu)-CO-(CH₂)₁₆-CO₂H)AQ-Aib-EFIe-αMeY- | | | |
| | | | LIEGSPSSGAPPPSK(PEG₂₄-BEA-MalDap₂-Fc)-NH₂ | | | |
| 234 | | 692 | Y-Aib-EGT-αMeF(2F)-TSDY-Aib-I-αMeL-LD-Orn-K(2-[2-(2-amino- | 61 | 310 | 935 |
| | | | ethoxy)-ethoxy]-acetyl)-(γGlu)-CO-(CH₂)16-CO₂H)AQ-Aib-EFIe-αMeY- | | | |
| | | | LIEGSPSSGAPPPSK(PEG₂₄-BEA-MalDap₂-Fc)-NH₂ | | | |
| 235 | | 693 | Y-Aib-EGT-αMeF(2F)-TSD-4Pal-SI-αMeL-LD-Orn-K(ϵKϵK-(γGlu)-CO- | 87 | 311 | 935 |
| | | | (CH₂)₁₆-CO₂H)AQ-Aib-EFIe-αMeY-LVEGSPSSGAPPPSK(PEG₂₄-BEA- | | | |
| | | | MalDap₂-Fc)-NH₂ | | | |
| 236 | | 694 | Y-Aib-EGT-αMeF(2F)-TSD-4Pal-SI-αMeL-LD-Orn-K(2-[2-(2-amino- | 88 | 312 | 935 |
| | | | ethoxy)-ethoxy]-acetyl)₂-(γGlu)-CO-(CH₂)₁₆-CO₂H)AQ-Aib-EFIE-αMeY- | | | |
| | | | LIAGSPSSGAPPPSK(PEG₂₄-BEA-MalDap₂-Fc)-NH₂ | | | |
| 237 | | 695 | Y-Aib-EGT-αMeF(2F)-TSD-4Pal-SI-αMeL-LD-Orn-K(ϵKϵK-(γGlu)-CO- | 88 | 313 | 935 |
| | | | (CH₂)₁₆-CO₂H)AQ-Aib-EFIE-αMeY-LIAGSPSSGAPPPSK(PEG₂₄-BEA- | | | |
| | | | MalDap₂-Fc)-NH₂ | | | |
| 238 | | 696 | Y-Aib-EGT-αMeF(2F)-TSDY-Aib-I-αMeL-LD-Orn-K(2-[2-(2-amino- | 89 | 314 | 935 |
| | | | ethoxy)-ethoxy]-acetyl)₂-(γGlu)-CO-(CH₂)₁₆-CO₂H)AQ-Aib-EFIE-αMeY- | | | |
| | | | LIAGSPSSGAPPPSK(PEG₂₄-BEA-MalDap₂-Fc)-NH₂ | | | |
| 239 | | 697 | Y-Aib-EGT-αMeF(2F)-TSDY-Aib-I-αMeL-LD-Orn-K(ϵKϵK-(γGlu)-CO- | 89 | 315 | 935 |
| | | | (CH₂)₁₆-CO₂H)AQ-Aib-EFIE-αMeY-LIAGSPSSGAPPPSK(PEG₂₄-BEA- | | | |
| | | | MalDap₂-Fc)-NH₂ | | | |
| 480 | 22 | 698 | Y-Aib-EGT-αMeF(2F)-TSD-4Pal-SI-αMeL-LD-Orn-K(ϵKK-(γGlu)-CO- | 2 | 258 | 935 |
| | | | (CH₂)₁₆-CO₂H)AQ-Aib-EFIe-αMeY-LIEGSPSSGAPPPSK(PEG₂₄-BEA- | | | |
| | | | MSPT2-Fc)-NH₂ | | | |
| 481 | 23 | 699 | Y-Aib-EGT-αMeF(2F)-TSD-4Pal-SI-αMeL-LD-Orn-K(γGlu-(γGlu)-CO- | 2 | 258 | 935 |
| | | | (CH₂)₁₆-CO₂H)AQ-Aib-EFIe-αMeY-LIEGSPSSGAPPPSK(PEG₂₄-BEA- | | | |
| | | | Acetyl2-Fc)-NH₂ | | | |
| 482 | 24 | 700 | Y-Aib-EGT-αMeF(2F)-TSD-4Pal-SI-αMeL-LD-Orn-K(γGlu-(γGlu)-CO- | 2 | 258 | 935 |
| | | | (CH₂)₁₆-CO₂H)AQ-Aib-EFIe-αMeY-LIEGSPSSGAPPPSK(PEG₂₄-BEA-OD2- | | | |
| | | | Fc)-NH₂ | | | |
| 243 | | 701 | Y-Aib-EGT-αMeF(2F)-TSD-4Pal-SI-αMeL-LDEK(Ac)AQ-Aib-EFIe-αMeY- | 1 | 316 | 935 |
| | | | LIEGSPSSGAPPPSK(PEG₂₄-BEA-MalDap₂-Fc)-NH₂ | | | |
| 244 | | 702 | Y-Aib-EGT-αMeF(2F)-TSD-4Pal-SI-αMeL-LDEQAQ-Aib-EFIe-αMeY- | 90 | 317 | 935 |
| | | | LIEGSPSSGAPPPSK(PEG₂₄-BEA-MalDap₂-Fc)-NH₂ | | | |
| 245 | | 703 | Y-Aib-EGT-αMeF(2F)-TSD-4Pal-SI-αMeL-LDEQAQ-Aib-EFIe-αMeY- | 90 | 317 | 936 |
| | | | LIEGSPSSGAPPPSK(PEG₂₄-BEA-MalDap₂-Fc KRQ)-NH₂ | | | |
| 246 | | 704 | Y-Aib-EGT-αMeF(2F)-TSD-4Pal-SI-αMeL-LD-Orn-K(2-[2-(2-amino- | 2 | 318 | 935 |
| | | | ethoxy)-ethoxy]-acetyl)₂-(γGlu)-CO-(CH₂)₁₈-CO₂H)AQ-Aib-EFIe-αMeY- | | | |
| | | | LIEGSPSSGAPPPSK(PEG24-BEA-MalDap2-Fc)-NH₂ | | | |
| 247 | | 705 | Y-Aib-EGT-αMeF(2F)-TSD-4Pal-SI-αMeL-LD-Orn-K(2-[2-(2-amino- | 2 | 319 | 935 |
| | | | ethoxy)-ethoxy]-acetyl)₂-(γGlu)-CO-(CH₂)₁₄-CO₂H)AQ-Aib-EFIe-αMeY- | | | |
| | | | LIEGSPSSGAPPPSK(PEG₂₄-BEA-MalDap₂-Fc)-NH₂ | | | |
| 248 | | 706 | Y-Aib-EGT-αMeF(2F)-TSD-4Pal-SI-αMeL-LDEK(2-[2-(2-amino-ethoxy)- | 1 | 320 | 935 |
| | | | ethoxy]-acetyl)₂-(γGlu)-CO-(CH₂)₁₄-CO₂H)AQ-Aib-EFIe-αMeY- | | | |
| | | | LIEGSPSSGAPPPSK(PEG₂₄-BEA-MalDap₂-Fc)-NH₂ | | | |
| 249 | | 707 | Y-Aib-EGT-aMcF(2F)-TSD-4Pal-SI-αMeL-LD-Orn-K(γGlu-(γGlu)-CO- | 2 | 321 | 935 |
| | | | (CH₂)₁₈-CO₂H)AQ-Aib-EFIe-αMeY-LIEGSPSSGAPPPSK(PEG2₄-BEA- | | | |
| | | | MalDap₂-Fc)-NH₂ | | | |
| 250 | | 708 | Y-Aib-EGT-αMeF(2F)-TSD-4Pal-SI-αMeL-LDEK(γGlu-(γGlu)-CO- | 1 | 322 | 935 |
| | | | (CH₂)₁₈-CO₂H)AQ-Aib-EFIe-αMeY-LIEGSPSSGAPPPSK(PEG₂₄-BEA- | | | |
| | | | MalDap₂-Fc)-NH₂ | | | |
| 251 | | 709 | Y-Aib-EGT-αMeF(2F)-TSD-4Pal-SI-αMeL-LD-Orn-K(2-[2-(2-amino- | 21 | 323 | 935 |
| | | | ethoxy)-ethoxy]-acetyl)₂-(γGlu)-CO-(CH₂)₁₄-CO₂H)AQ-Aib-EFIE-αMeY- | | | |
| | | | LIEGGGGGGGSGGGGSGGGGSK(BEA-MalDap₂-Fc)-NH₂ | | | |
| 252 | | 710 | Y-Aib-EGT-αMeF(2F)-TSD-4Pal-SI-αMeL-LDEK(γGlu-(2-[2-(2-amino- | 1 | 324 | 935 |
| | | | ethoxy)-ethoxy]-acetyl)-ϵK-CO-(CH₂)₁₈-CO₂H)AQ-Aib-EFIe-αMeY- | | | |
| | | | LIEGSPSSGAPPPS-K(PEG₂₄-BEA-MalDap₂-Fc)-NH₂ | | | |
| 253 | | 711 | Y-Aib-EGT-αMeF(2F)-TSD-4Pal-Aib-I-αMeL-LD-Orn-K(2-12-(2-amino- | 22 | 325 | 935 |
| | | | ethoxy)-ethoxy]-acetyl)₂-(γGlu)-CO-(CH₂)₁₄-CO₂H)AQ-Aib-EFIE-αMeY- | | | |
| | | | LIEGGGGGGGSGGGGSGGGGSK(BEA-MalDap₂-Fc)-NH₂ | | | |
| 254 | | 712 | Y-Aib-EGT-αMeF(2F)-TSD-4Pal-Aib-I-αMeL-LD-Orn-K(2-[2-(2-amino- | 42 | 326 | 935 |
| | | | ethoxy)-ethoxy]-acetyl)₂-(γGlu)-CO-(CH₂)₁₄-CO₂H)AQ-Aib-EFIE-αMeY- | | | |
| | | | LIEGGPSSGK(PEG₂₄-BEA-MalDap₂-Fc)-NH₂ | | | |
| 255 | | 713 | Y-Aib-EGT-αMeF(2F)-TSD-4Pal-Aib-I-αMeL-LD-Orn-K(2-[2-(2-amino- | 22 | 325 | 935 |
| | | | ethoxy)-ethoxy]-acetyl)₂-(γGlu)-CO-(CH₂)₁₄-CO₂H)AQ-Aib-EFIE-αMeY- | | | |
| | | | LIEGGG-PEG₂₄-K(BEA-MalDap₂-Fc)-NH₂ | | | |
| 256 | | 714 | Y-Aib-EGT-αMeF(2F)-TSD-4Pal-Aib-I-αMeL-LD-Orn-K(2-[2-(2-amino- | 22 | 325 | 935 |
| | | | ethoxy)-ethoxy]-acetyl)₂-(γGlu)-CO-(CH₂)₁₄-CO₂H)AQ-Aib-EFIE-αMeY- | | | |
| | | | LIEGGGK(PEG₂₄-BEA-MalDap₂-Fc)-NH₂ | | | |
| 257 | | 715 | Y-Aib-EGT-αMeF(2F)-TSD-4Pal-Aib-I-αMeL-LDEK(γGlu-(γGlu)-CO- | 91 | 327 | 935 |
| | | | (CH₂)₁₄-CO₂H)AQ-Aib-EFIE-αMeY-LIEGGPSSGK(PEG₂₄-BEA-MalDap₂- | | | |
| | | | Fc)-NH₂ | | | |
| 258 | | 716 | Y-Aib-EGT-αMeF(2F)-TSD-4Pal-Aib-I-αMeL-LDEK(γGlu-(γGlu)-CO- | 16 | 328 | 935 |
| | | | (CH₂)₁₄-CO₂H)AQ-Aib-EFIE-αMeY-LIEGGG-PEG₂₄-K(BEA-MalDap₂-Fc)- | | | |
| | | | NH₂ | | | |
| 259 | | 717 | Y-Aib-EGT-αMeF(2F)-TSD-4Pal-Aib-I-αMeL-LDEK(ϵKϵK-(γGlu)-CO- | 16 | 328 | 935 |
| | | | (CH₂)₁₄-CO₂H)AQ-Aib-EFIE-αMeY-LIEGGGK(PEG₂₄-BEA-MalDap₂-Fc)- | | | |
| | | | NH₂ | | | |
| 260 | | 718 | Y-Aib-EGT-αMeF(2F)-TSD-4Pal-SI-αMeL-LDEK(2-[2-(2-amino-ethoxy)- | 15 | 329 | 935 |
| | | | ethoxy]-acetyl)₂-(γGlu)-CO-(CH₂)₁₄-CO₂H)AQ-Aib-EFIE-αMeY- | | | |
| | | | LIEGGGGGGGSGGGGSGGGGSK(BEA-MalDap₂-Fc)-NH₂ | | | |
| 261 | | 719 | Y-Aib-EGT-αMeF(2F)-TSD-4Pal-SI-αMeL-LDEK(2-[2-(2-amino-ethoxy)- | 15 | 325 | 935 |
| | | | ethoxy]-acetyl)₂-γGlu-(ϵKϵK-(γGlu)-CO-(CH₂)₁₄-CO₂H))AQ-Aib-EFIE- | | | |
| | | | αMeY-LIEGGGGGGGSGGGGSGGGGSK(BEA-MalDap₂-Fc)-NH₂ | | | |
| 262 | | 720 | Y-Aib-EGT-αMeF(2F)-TSD-4Pal-SI-αMeL-LDEK(2-[2-(2-amino-ethoxy)- | 1 | 187 | 936 |
| | | | ethoxy]-acetyl)₂-(γGlu)-CO-(CH₂)₁₆-CO2H)AQ-Aib-EFIe-αMeY- | | | |
| | | | LIEGSPSSGAPPPSK((2-[2-(2-amino-ethoxy)-ethoxy]-acetyl)₄- | | | |
| | | | GKAAAEKAAAEKAAAE-BEA(MalDap₂-Fc KRQ)-NH₂ | | | |
| 263 | | 721 | Y-Aib-EGT-αMeF(2F)-TSDV-Aib-I-αMeL-LD-EK(2-[2-(2-amino-ethoxy)- | 8 | 194 | 936 |
| | | | ethoxy]-acetyl)₂-(γGlu)-CO-(CH₂)₁₆-CO2H)AQ-Aib-EFIe-αMeY- | | | |
| | | | LIEGSPSSGAPPPSK(PEG₂₄-BEA-MalDap₂-Fc KRQ)-NH₂ | | | |
| 264 | | 722 | Y-Aib-EGT-αMeF(2F)-TSD-4Pal-SI-αMeL-LDEK(2-[2-(2-amino-ethoxy)- | 11 | 197 | 935 |
| | | | ethoxy]-acetyl)₂-(γGlu)-CO-(CH₂)₁₆-CO₂H)AQ-Aib-EFIK-αMeY- | | | |
| | | | LIEGSPSSGAPPPSGS-NH₂ | | | |
| 265 | | 723 | Y-Aib-EGT-αMeF(2F)-TSD-4Pal-SI-αMeL-LDEK(2-[2-(2-amino-ethoxy)- | 3 | 189 | 936 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGAPAPAPAPAPAPK(BEA-MalDap₂-Fc KRQ)- | | | |
| | | | NH₂ | | | |
| 266 | | 724 | Y-Aib-EGT-αMeF(2F)-TSD-4Pal-SI-αMeL-LD-Orn-K(ϵKϵK-(γGlu)-CO- | 2 | 258 | 936 |
| | | | (CH₂)₁₆-CO₂H)AQ-Aib-EFIe-αMeY-LIEGSPSSGAPPPS-K(PEG₂₄-BEA- | | | |
| | | | MalDap₂-Fc KRQ)-NH₂ | | | |
| 267 | | 725 | Y-Aib-EGT-αMeF(2F)-TSD-V-Aib-I-αMeL-LD-Orn-K(2-12-(2-amino- | 19 | 206 | 936 |
| | | | ethoxy)-ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSK(BEA-MalDap₂-Fc KRQ)-NH₂ | | | |
| 268 | | 726 | Y-Aib-EGT-αMeF(2F)-TSD-4Pal-SI-αMeL-LD-Orn-K(2-[2-(2-amino- | 21 | 208 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSK(BEA-MalDap₂-Fc KRQ)-NH₂ | | | |
| 269 | | 727 | Y-Aib-EGT-aMcF(2F)-TSD-4Pal-SI-αMeL-LDEK(2-[2-(2-amino-cthoxy)- | 3 | 189 | 936 |
| | | | ethoxy]-acetyl)₂-(γGlu)-CO-(CH₂)₁₆-CO₂H)AQ-Aib-EFIe-αMeY- | | | |
| | | | LIEGGGGGGGSGGGGSGGGGSGAPAPAPAPAPAPK(BEA-MalDap₂-Fc | | | |
| | | | KRQ)-NH₂ | | | |
| 270 | | 728 | Y-Aib-EGT-αMeF(2F)-TSD-4Pal-SI-αMeL-LDEK(γGlu-(2-[2-(2-amino- | 1 | 324 | 936 |
| | | | ethoxy)-ethoxy]-acetyl)-ϵK-CO-(CH₂)₁₈-CO₂H)AQ-Aib-EFIe-αMeY- | | | |
| | | | LIEGSPSSGAPPPS-K(PEG₂₄-BEA-MalDap₂-Fc KRQ)-NH₂ | | | |
| 271 | | 729 | Y-Aib-EGT-αMeF(2F)-TSD-4Pal-Aib-I-αMeL-LDEK(2-[2-(2-amino- | 16 | 203 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGAPAPAPAPAPAPK(BEA-MalDap₂-Fc | | | |
| | | | KRQ)-NH₂ | | | |
| 272 | | 730 | Y-Aib-EGT-αMeF(2F)-TSD-4Pal-Aib-I-αMeL-LDEK(2-[2-(2-amino- | 16 | 203 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEAAAKEAAAKEAAAKK(BEA- | | | |
| | | | MalDap₂-Fc KRQ)-NH₂ | | | |
| 273 | | 731 | Y-Aib-EGT-αMeF(2F)-TSD-4Pal-SI-αMeL-LEEK(2-[2-(2-amino-ethoxy)- | 17 | 204 | 936 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGAPAPAPAPAPAPK(BEA-MalDap₂-Fc | | | |
| | | | KRQ)-NH₂ | | | |
| 274 | | 732 | Y-Aib-EGT-αMeF(2F)-TSD-4Pal-SI-αMeL-LEEK(2-[2-(2-amino-ethoxy)- | 17 | 204 | 936 |
| | | | ethoxy]-acetyl)₂-(γGlu)-CO-(CH₂)₁₆-CO₂H)AQ-Aib-EFIE-αMeY- | | | |
| | | | LIEGGGGGGGSGEAAAKEAAAKEAAAKK(BEA-MalDap₂-Fc KRQ)-NH₂ | | | |
| 275 | | 733 | Y-Aib-EGT-αMeF(2F)-TSD-4Pal-SI-αMeL-LEEK(2-[2-(2-amino-ethoxy)- | 17 | 204 | 936 |
| | | | ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEPEPEPEPEPEPK(BEA-MalDap₂-Fc | | | |
| | | | KRQ)-NH₂ | | | |
| 276 | | 734 | Y-Aib-EGT-αMeF(2F)-TSD-4Pal-SI-αMeL-LEEK(2-[2-(2-amino-ethoxy)- | 17 | 204 | 936 |
| | | | ethoxy]-acetyl)₂-(γGlu)-CO-(CH₂)₁₆-CO₂H)AQ-Aib-EFIE-αMeY- | | | |
| | | | LIEGGGGGGGQGGGGQGGGGQGEPEPEPEPEPEPK(BEA-MalDap₂-Fc | | | |
| | | | KRQ)-NH₂ | | | |
| 277 | | 735 | Y-Aib-EGT-αMeF(2F)-TSD-4Pal-SI-αMeL-LE-Orn-K(2-[2-(2-amino- | 23 | 210 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGEPEPEPEPEPEPK(BEA-MalDap₂-Fc KRQ)-NH₂ | | | |
| 278 | | 736 | Y-Aib-EGT-αMeF(2F)-TSD-4Pal-SI-αMeL-LE-Orn-K(2-[2-(2-amino- | 23 | 210 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGGGGSGEPEPEPEPEPEPK(BEA-MalDap₂-Fc | | | |
| | | | KRQ)-NH₂ | | | |
| 279 | | 737 | Y-Aib-EGT-αMeF(2F)-TSD-4Pal-SI-αMeL-LE-Orn-K(2-[2-(2-amino- | 25 | 212 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPS-K(2-[2-(2-amino-ethoxy)-ethoxy]-acetyl)₆- | | | |
| | | | GPAPAPAPAPAPA-BEA(MalDap₂-Fc KRQ)-NH₂ | | | |
| 280 | | 738 | Y-Aib-EGT-αMeF(2F)-TSD-4Pal-Aib-I-αMeL-LD-Orn-K(2-[2-(2-amino- | 22 | 209 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc KRQ)- | | | |
| | | | NH₂ | | | |
| 281 | | 739 | Y-Aib-EGT-αMeF(2F)-TSD-4Pal-SI-αMeL-LE-Orn-K(2-[2-(2-amino- | 23 | 210 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc KRQ)- | | | |
| | | | NH₂ | | | |
| 282 | | 740 | Y-Aib-EGT-αMeF(2F)-TSD-4Pal-Aib-I-αMeL-LD-Orn-K(2-[2-(2-amino- | 27 | 214 | 936 |
| | | | ethoxy)-ethoxy]-acetyl)₂-(γGlu)-CO-(CH₂)₁₆-CO₂H)AQ-Aib-EFIe-αMeY- | | | |
| | | | LIEGGGGGGGSGGGGSGAPAPAPAPAPAPK(BEA-MalDap₂-Fc KRQ)- | | | |
| | | | NH₂ | | | |
| 283 | | 741 | Y-Aib-EGT-αMeF(2F)-TSD-4Pal-SI-αMeL-LD-Orn-K(2-[2-(2-amino- | 28 | 215 | 936 |
| | | | ethoxy)-ethoxy]-acetyl)₂-(γGlu)-CO-(CH₂)₁₆-CO₂H)AQ-Aib- | | | |
| | | | EFIK(GGGGSGGGGSGAPAPAPAPAPAPG-BEA-MalDap₂-Fc KRQ)- | | | |
| | | | αMeY-LIEGSPSSGAPPPSGS-NH₂ | | | |
| 284 | | 742 | Y-Aib-EGT-αMeF(2F)-TSD-4Pal-Aib-I-αMeL-LD-Orn-K(2-12-(2-amino- | 29 | 216 | 936 |
| | | | ethoxy)-ethoxy]-acetyl)₂-(γGlu)-CO-(CH₂)₁₆-CO₂H)AQ-Aib- | | | |
| | | | EFIK(GGGGSGGGGSGAPAPAPAPAPAPG-BEA-MalDap₂-Fc KRQ)- | | | |
| | | | αMeY-LIEGSPSSGAPPPSGS-NH₂ | | | |
| 285 | | 743 | Y-Aib-EGT-αMeF(2F)-TSD-4Pal-Aib-I-αMeL-LD-Orn-K(ϵKϵK-(γGlu)-CO- | 42 | 230 | 936 |
| | | | (CH₂)₁₆-CO₂H)AQ-Aib-EFIE-αMeY-LIEGGPSSGK(PEG₂₄-BEA-MalDap₂- | | | |
| | | | Fc KRQ)-NH₂ | | | |
| 286 | | 744 | Y-Aib-EGT-αMeF(2F)-TSD-4Pal-Aib-I-αMeL-LD-Orn-K(2-[2-(2-amino- | 42 | 239 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGPSSGK(PEG₂₄-BEA-MalDap₂-Fc KRQ)-NH₂ | | | |
| 287 | | 745 | Y-Aib-EGT-αMeF(2F)-TSD-4Pal-Aib-I-αMeL-LD-Orn-K(2-[2-(2-amino- | 22 | 209 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGG-PEG₂₄-K(BEA-MalDap₂-Fc KRQ)-NH₂ | | | |
| 288 | | 746 | Y-Aib-EGT-αMeF(2F)-TSD-4Pal-Aib-I-αMeL-LD-Orn-K(2-[2-(2-amino- | 22 | 325 | 936 |
| | | | ethoxy)-ethoxy]-acetyl)₂-(γGlu)-CO-(CH₂)₁₄-CO₂H)AQ-Aib-EFIE-αMeY- | | | |
| | | | LIEGGGGGGGSGGGGSGGGGSK(BEA-MalDap₂-Fc KRQ)-NH₂ | | | |
| 289 | | 747 | Y-Aib-EGT-αMeF(2F)-TSD-4Pal-Aib-I-αMeL-LD-Orn-K(2-[2-(2-amino- | 47 | 240 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-MalDap₂-Fc KRQ)-NH₂ | | | |
| 290 | | 748 | Y-Aib-EGT-αMeF(2F)-TSD-4Pal-Aib-I-αMeL-LD-Orn-K(ϵKϵK-(γGlu)-CO- | 47 | 241 | 936 |
| | | | (CH₂)₁₆-CO₂H)AQ-Aib-EFIE-αMeY-LIEGSPSSGAPPPSK(PEG₂₄-BEA- | | | |
| | | | MalDap₂-Fc KRQ)-NH₂ | | | |
| 291 | | 749 | Y-Aib-EGT-αMeF(2F)-TSD-4Pal-Aib-I-αMeL-LDEK(ϵKϵK-(γGlu)-CO- | 5 | 242 | 936 |
| | | | (CH₂)₁₆-CO₂H)AQ-Aib-EFIE-αMeY-LIEGSPSSGAPPPSK(PEG₂₄-BEA- | | | |
| | | | MalDap₂-Fc KRQ)-NH₂ | | | |
| 292 | | 750 | Y-Aib-EGT-αMeF(2F)-TSD-4Pal-Aib-I-αMeL-LD-Orn-K(2-[2-(2-amino- | 48 | 243 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGGPSSGAPPPSK(PEG₂₄-BEA-MalDap₂-Fc KRQ)-NH₂ | | | |
| 293 | | 751 | Y-Aib-EGT-αMeF(2F)-TSD-4Pal-Aib-I-αMeL-LD-Orn-K(ϵKϵK-(γGlu)-CO- | 48 | 244 | 936 |
| | | | (CH₂)₁₆-CO₂H)AQ-Aib-EFIE-αMeY-LIEGGPSSGAPPPSK(PEG₂₄-BEA- | | | |
| | | | MalDap₂-Fc KRQ)-NH₂ | | | |
| 294 | | 752 | Y-Aib-EGT-αMeF(2F)-TSD-4Pal-Aib-I-αMeL-LDEK(ϵKϵK-(γGlu)-CO- | 49 | 245 | 936 |
| | | | (CH₂)₁₆-CO₂H)AQ-Aib-EFIE-αMeY-LIEGGPSSGAPPPSK(PEG₂₄-BEA- | | | |
| | | | MalDap₂-Fc KRQ)-NH₂ | | | |
| 295 | | 753 | Y-Aib-EGT-αMeF(2F)-TSD-4Pal-Aib-I-αMeL-LD-Orn-K(ϵKϵK-(γGlu)-CO- | 50 | 246 | 936 |
| | | | (CH₂)₁₆-CO₂H)AQ-Aib-EFIe-αMeY-LIEGSPSSGAPPPSK(PEG₂₄-BEA- | | | |
| | | | MalDap₂ Fc KRQ)-NH₂ | | | |
| 296 | | 754 | Y-Aib-EGT-αMeF(2F)-TSD-4Pal-Aib-I-αMeL-LD-Orn-K(ϵKϵK-(γGlu)-CO- | 27 | 247 | 936 |
| | | | (CH₂)₁₆-CO₂H)AQ-Aib-EFIe-αMeY-LIEGGGK(PEG₂₄-BEA-MalDap2 Fc | | | |
| | | | KRQ)-NH₂ | | | |
| 297 | | 755 | Y-Aib-EGT-αMeF(2F)-TSD-4Pal-Aib-I-αMeL-LD-Orn-K(ϵKϵK-(γGlu)-CO- | 51 | 248 | 936 |
| | | | (CH₂)₁₆-CO₂H)AQ-Aib-EFIE-αMeY-LIEGG-Orn-SSGAPPPSK(PEG₂₄-BEA- | | | |
| | | | MalDap₂ Fc KRQ)-NH₂ | | | |
| 298 | | 756 | Y-Aib-EGT-αMeF(2F)-TSD-4Pal-Aib-I-αMeL-LD-Orn-K(2-12-(2-amino- | 51 | 249 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGG-Orn-SSGAPPPSK(PEG₂₄-BEA-MalDap₂ Fc KRQ)-NH₂ | | | |
| 299 | | 757 | Y-Aib-EGT-αMeF(2F)-TSD-4Pal-Aib-I-αMeL-LD-Orn-K(ϵKϵK-(γGlu)-CO- | 52 | 250 | 936 |
| | | | (CH₂)₁₆-CO₂H)AQ-Aib-EFIE-αMeY-LIEGG-Orn-K(PEG₂₄-BEA-MalDap₂ Fc | | | |
| | | | KRQ)-NH₂ | | | |
| 300 | | 758 | Y-Aib-EGT-αMeF(2F)-TSD-4Pal-Aib-I-αMeL-LD-Orn-K(2-12-(2-amino- | 52 | 251 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGG-Orn-K(PEG₂₄-BEA-MalDap₂ Fc KRQ)-NH₂ | | | |
| 301 | | 759 | Y-Aib-EGT-αMeF(2F)-TSD-4Pal-Aib-I-αMeL-LD-Orn-K(ϵKϵK-(γGlu)-CO- | 53 | 252 | 936 |
| | | | (CH₂)₁₆-CO₂H)AQ-Aib-Orn-FIE-αMeY-LIEGSPSSGAPPPSK(PEG₂₄-BEA- | | | |
| | | | MalDap₂ Fc KRQ)-NH₂ | | | |
| 302 | | 760 | Y-Aib-EGT-αMeF(2F)-TSD-4Pal-Aib-I-αMeL-LD-Orn-K(2-[2-(2-amino- | 53 | 253 | 936 |
| | | | ethoxy)-ethoxy]-acetyl)₂-(γGlu)-CO-(CH₂)₁₆-CO₂H)AQ-Aib-Orn-FIE-αMeY- | | | |
| | | | LIEGSPSSGAPPPSK(PEG₂₄-BEA-MalDap₂ Fc KRQ)-NH₂ | | | |
| 303 | | 761 | Y-Aib-EGT-αMeF(2F)-TSD-4Pal-Aib-I-αMeL-LD-Orn-K(2-[2-(2-amino- | 54 | 254 | 936 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap2 Fc KRQ)-NH₂ | | | |
| 304 | | 762 | Y-Aib-EGT-αMeF(2F)-TSD-4Pal-Aib-I-αMeL-LD-Orn-K(ϵKϵK-(γGlu)-CO- | 54 | 255 | 936 |
| | | | (CH₂)₁₆-CO₂H)AQ-Aib-EFIQ-αMeY-LIEGSPSSGAPPPSK(PEG₂₄-BEA- | | | |
| | | | MalDap₂ Fc KRQ)-NH₂ | | | |
| 305 | | 763 | Y-Aib-EGT-αMeF(2F)-TSD-4Pal-Aib-I-αMeL-LD-Orn-K(2-[2-(2-amino- | 47 | 240 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc KRQ)-NH₂ | | | |
| 306 | | 764 | Y-Aib-EGT-αMeF(2F)-TSD-4Pal-SI-αMeL-LD-Orn-K(2-[2-(2-amino- | 55 | 256 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc KRQ)-NH₂ | | | |
| 307 | | 765 | Y-Aib-EGT-αMeF(2F)-TSD-4Pal-SI-αMeL-LD-Orn-K(ϵKϵK-(γGlu)-CO- | 55 | 257 | 936 |
| | | | (CH₂)₁₆-CO₂H)AQ-Aib-EFIE-αMeY-LIEGSPSSGAPPPSK(2-[2-(2-amino- | | | |
| | | | ethoxy)-ethoxy]-acetyl)₆-GPAPAPAPAPAPA-BEA-MalDap₂ Fc KRQ)-NH₂ | | | |
| 308 | | 766 | Y-Aib-EGT-αMeF(2F)-TSD-4Pal-SI-αMeL-LD-Orn-K(ϵKϵK-(γGlu)-CO- | 2 | 258 | 936 |
| | | | (CH₂)₁₆-CO₂H)AQ-Aib-EFIe-αMeY-LIEGSPSSGAPPPSK(2-[2-(2-amino- | | | |
| | | | ethoxy)-ethoxy]-acetyl)₆-GPAPAPAPAPAPA-BEA-MalDap₂-Fc KRQ)-NH₂ | | | |
| 309 | | 767 | Y-Aib-EGT-αMeF(2F)-TSD-4Pal-Aib-I-αMeL-LD-Orn-K(2-[2-(2-amino- | 47 | 240 | 936 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆-BEA- | | | |
| | | | MalDap₂-Fc KRQ)-NH₂ | | | |
| 310 | | 768 | Y-Aib-EGT-αMeF(2F)-TSD-4Pal-SI-αMeL-LD-Orn-K(ϵKϵK-(γGlu)-CO- | 2 | 258 | 941 |
| | | | (CH₂)₁₆-CO2H)AQ-Aib-EFIe-αMeY-LIEGSPSSGAPPPSK(PEG₂₄-BEA- | | | |
| | | | MalDap₂-Fc IG KRQ)-NH₂ | | | |
| 311 | | 769 | Y-Aib-EGT-αMeF(2F)-TSD-4Pal-SI-αMeL-LE-Orn-K(2-[2-(2-amino- | 23 | 210 | 94 |
| | | | ethoxy)-ethoxy]-acetyl)₂-(γGlu)-CO-(CH₂)₁₆-CO₂H)AQ-Aib-EFIE-αMeY- | | | |
| | | | LIEGGGGGGGSGGGGSGAPAPAPAPAPAPK(BEA-MalDap₂-Fc IG | | | |
| | | | KRQ)-NH₂ | | | |
| 312 | | 770 | Y-Aib-EGT-αMeF(2F)-TSD-4Pal-Aib-I-αMeL-LD-Orn-K(2-[2-(2-amino- | 54 | 254 | 941 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂ Fc IG KRQ)-NH₂ | | | |
| 313 | | 771 | Y-Aib-EGT-αMeF(2F)-TSD-4Pal-Aib-I-αMeL-LD-Orn-K(ϵKϵK-(γGlu)-CO- | 54 | 255 | 941 |
| | | | (CH₂)₁₆-CO₂H)AQ-Aib-EFIQ-αMeY-LIEGSPSSGAPPPSK(PEG₂₄-BEA- | | | |
| | | | MalDap₂ Fc IG KRQ)-NH₂ | | | |
| 314 | | 772 | Y-Aib-EGT-αMeF(2F)-TSD-4Pal-Aib-I-αMeL-LD-Orn-K(2-[2-(2-amino- | 47 | 240 | 941 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(2-[2-(2-amino-ethoxy)-ethoxy]-acetyl)₆-BEA- | | | |
| | | | MalDap₂-Fc IG KRQ)-NH₂ | | | |
| 315 | | 773 | Y-Aib-EGT-αMeF(2F)-TSD-4Pal-SI-αMeL-LD-Orn-K(2-[2-(2-amino- | 58 | 262 | 941 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂-Fc IG KRQ)-NH₂ | | | |
| 316 | | 774 | Y-Aib-EGT-αMeF(2F)-TSDY-Aib-I-αMeL-LD-Orn-K(2-[2-(2-amino- | 61 | 330 | 941 |
| | | | ethoxy)-ethoxy]-acetyl)₂-(γGlu)-CO-(CH₂)₁₆-CO₂H)AQ-Aib-EFIe-αMeY- | | | |
| | | | LIEGSPSSGAPPPSK(PEG₂₄-BEA-MalDap₂-Fc IG KRQ)-NH₂ | | | |
| 317 | | 775 | Y-Aib-EGT-αMeF(2F)-TSDY-Aib-I-αMeL-LD-Orn-K(2-[2-(2-amino- | 59 | 264 | 941 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-MalDap₂-Fc IG KRQ)-NH₂ | | | |
| 318 | | 776 | Y-Aib-EGT-αMeF(2F)-TSD-4Pal-Aib-I-αMeL-LD-Orn-K(2-[2-(2-amino- | 92 | 331 | 941 |
| | | | ethoxy)-ethoxy]-acetyl)₂-(γGlu)-CO-(CH₂)₁₆-CO₂H)AQ-Aib-EFIQ-αMeY- | | | |
| | | | LIE-Aib-SPSSGAPPPSK(PEG₂₄-BEA-MalDap₂-Fc IG KRQ)-NH₂ | | | |
| 319 | | 777 | Y-Aib-EGT-αMeF(2F)-TSD-4Pal-Aib-I-αMeL-LD-Orn-K(ϵKϵK-(γGlu)-CO- | 92 | 332 | 941 |
| | | | (CH₂)₁₆-CO₂H)AQ-Aib-EFIQ-αMeY-LIE-Aib-SPSSGAPPPSK(PEG₂₄-BEA- | | | |
| | | | MalDap₂-Fc IG KRQ)-NH₂ | | | |
| 320 | | 778 | Y-Aib-EGT-αMeF(2F)-TSD-4Pal-SI-αMeL-LD-Orn-K(2-[2-(2-amino- | 58 | 262 | 941 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆- | | | |
| | | | GPAPAPAPAPAPA-BEA-MalDap₂-Fc IG KRQ)-NH₂ | | | |
| 321 | | 779 | Y-Aib-EGT-αMeF(2F)-TSD-4Pal-SI-αMeL-LD-Orn-K(ϵKϵK-(γGlu)-CO- | 58 | 263 | 941 |
| | | | (CH₂)₁₆-CO₂H)AQ-Aib-EFIQ-αMeY-LIEGSPSSGAPPPSK(2-12-(2-amino- | | | |
| | | | ethoxy)-ethoxy]-acetyl)₆-GPAPAPAPAPAPA-BEA-MalDap₂-Fc IG KRQ)- | | | |
| | | | NH₂ | | | |
| 322 | | 780 | Y-Aib-EGT-αMeF(2F)-TSD-4Pal-SI-αMeL-LD-Orn-K(2-[2-(2-amino- | 58 | 262 | 941 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂-Fc IG KRQ)-NH₂ | | | |
| 323 | | 781 | Y-Aib-EGT-αMeF(2F)-TSD-4Pal-SI-αMeL-LD-Orn-K(ϵKϵK-(γGlu)-CO- | 58 | 263 | 941 |
| | | | (CH₂)₁₆-CO₂H)AQ-Aib-EFIQ-αMeY-LIEGSPSSGAPPPSK(PEG₂₄-BEA- | | | |
| | | | MalDap₂-Fc IG KRQ)-NH₂ | | | |
| 324 | | 782 | Y-Aib-EGT-αMeF(2F)-TSD-4Pal-SI-αMeL-LD-Orn-K(2-[2-(2-amino- | 58 | 262 | 941 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(2-[2-(2-amino-ethoxy)-ethoxy]-acetyl)₆-BEA- | | | |
| | | | MalDap₂-Fc IG KRQ)-NH₂ | | | |
| 325 | | 783 | Y-Aib-EGT-αMeF(2F)-TSD-4Pal-SI-αMeL-LD-Orn-K(ϵKϵK-(γGlu)-CO- | 58 | 263 | 941 |
| | | | (CH₂)₁₆-CO₂H)AQ-Aib-EFIQ-αMeY-LIEGSPSSGAPPPSK(2-[2-(2-amino- | | | |
| | | | ethoxy)-ethoxy]-acetyl)₆-BEA-MalDap₂-Fc IG KRQ)-NH₂ | | | |
| 326 | | 784 | Y-Aib-EGT-αMeF(2F)-TSD-4Pal-SI-αMeL-LD-Orn-K(ϵKϵK-(γGlu)-CO- | 2 | 258 | 935 |
| | | | (CH₂)₁₆-CO₂H)AQ-Aib-EFIe-αMeY-LIEGSPSSGAPPPS-K(PEG₂₄-BEA- | | | |
| | | | MalDap₂-Fc N-Cys)-NH₂ | | | |
| 327 | | 785 | Y-Aib-EGT-αMeF(2F)-TSD-4Pal-SI-αMeL-LD-Orn-K(ϵKϵK-(γGlu)-CO- | 2 | 258 | 942 |
| | | | (CH₂)₁₆-CO₂H)AQ-Aib-EFIe-αMeY-LIEGSPSSGAPPPS-K(PEG₂₄-BEA- | | | |
| | | | MalDap₂-Fc N-Cys KRQ)-NH₂ | | | |
| 328 | | 786 | Y-Aib-EGT-αMeF(2F)-TSD-4Pal-Aib-I-αMeL-LD-Orn-K(2-[2-(2-amino- | 27 | 214 | 936 |
| | | | ethoxy)-ethoxy]-acetyl)₂-(γGlu)-CO-(CH₂)₁₆-CO₂H)AQ-Aib-EFIe-αMeY- | | | |
| | | | LIEGGGGGGGSGGGGSGAPAPAPAPAPAPK(BEA-MalDap2-Fc KRQ)- | | | |
| | | | NH₂ | | | |
| 329 | | 787 | Y-Aib-EGT-αMeF(2F)-TSD-4Pal-SI-αMeL-LD-Orn-K(ϵKϵK-(γGlu)-CO- | 2 | 258 | 936 |
| | | | (CH₂)₁₆-CO₂H)AQ-Aib-EFIe-αMeY-LIEGSPSSGAPPPS-K(PEG₂₄-BEA- | | | |
| | | | MalDap₂-Fc KRQ)-NH₂ | | | |
| 330 | | 788 | Y-Aib-QGT-αMeF(2F)-TSD-4Pal-SI-αMeL-LD-Orn-K(2-[2-(2-amino- | 93 | 333 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc)-NH₂ | | | |
| 331 | | 789 | Y-Aib-QGT-αMeF(2F)-TSD-4Pal-SI-αMeL-LD-Orn-K(2-[2-(2-amino- | 94 | 334 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 332 | | 790 | Y-Aib-QGT-αMeF(2F)-TSD-4Pal-αMeS-I-αMeL-LD-Orn-K(2-[2-(2-amino- | 95 | 335 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc)-NH₂ | | | |
| 333 | | 791 | Y-Aib-QGT-αMeF(2F)-TSD-4Pal-αMeS-I-αMeL-LD-Orn-K(2-12-(2-amino- | 96 | 336 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 336 | 25 | 792 | Y-Aib-QGT-αMeF(2F)-TSDY-αMeS-I-αMeL-LD-Orn-K(2-12-(2-amino- | 97 | 337 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc)-NH₂ | | | |
| 337 | | 793 | Y-Aib-QGT-αMeF(2F)-TSDY-αMeS-I-αMeL-LD-Orn-K(2-[2-(2-amino- | 98 | 338 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 338 | | 794 | Y-Aib-QGT-αMeF(2F)-TSD-4Pal-αMeS-I-αMeL-LD-Orn-K(2-[2-(2-amino- | 99 | 339 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGGGGGGGSGGGGSGAPAPAPAPAPAPK(BEA-MalDap₂-Fc)- | | | |
| | | | NH₂ | | | |
| 339 | | 795 | Y-Aib-QGT-αMeF(2F)-TSDY-αMeS-I-αMeL-LD-Orn-K(2-[2-(2-amino- | 100 | 340 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGGGGGSGGGGSGAPAPAPAPAPAPK(BEA-MalDap₂-Fc)- | | | |
| | | | NH₂ | | | |
| 340 | | 796 | Y-Aib-QGT-αMeF(2F)-TSD-4Pal-αMeS-I-αMeL-LD-Orn-K(2-[2-(2-amino- | 101 | 341 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGGPSSGAPPPSGK(PEG₂₄-BEA-MalDap₂-Fc)-NH₂ | | | |
| 341 | | 797 | Y-Aib-QGT-αMeF(2F)-TSDY-αMeS-I-αMeL-LD-Orn-K(2-[2-(2-amino- | 102 | 342 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGPSSGAPPPSGK(PEG₂₄-BEA-MalDap₂-Fc)-NH2 | | | |
| 342 | | 798 | Y-Aib-QGT-αMeF(2F)-TSD-4Pal-αMeS-I-αMeL-LD-Orn-K(ϵKϵK-(γGlu)- | 101 | 343 | 935 |
| | | | CO-(CH₂)₁₆-CO₂H)AQ-αMe4Pal-Orn-FIE-αMeY- | | | |
| | | | LIEGGPSSGAPPPSGK(PEG₂₄-BEA-MalDap₂-Fc)-NH₂ | | | |
| 343 | | 799 | Y-Aib-QGT-αMeF(2F)-TSDY-αMeS-I-αMeL-LD-Orn-K(&K&K-(γGlu)-CO- | 102 | 344 | 935 |
| | | | (CH₂)₁₆-CO₂H)AQ-αMe4Pal-Orn-FIe-αMeY-LIEGGPSSGAPPPSGK(PEG₂₄- | | | |
| | | | BEA-MalDap₂-Fc)-NH₂ | | | |
| 344 | | 800 | Y-Aib-QGT-αMeF(2F)-TSD-4Pal-αMeS-I-αMeL-LD-Orn-K(2-[2-(2-amino- | 103 | 345 | 935 |
| | | | ethoxy)-ethoxy]-acetyl)₂-(γGlu)-CO-(CH₂)₁₆-CO₂H)AQ-αMe4Pal-Orn- | | | |
| | | | FIK(PEG₂₄-BEA-MalDap₂-Fc)-αMeY-LIEGGPSSGAPPPSGG-NH₂ | | | |
| 345 | | 801 | Y-Aib-QGT-αMeF(2F)-TSDY-αMeS-I-αMeL-LD-Orn-K(2-[2-(2-amino- | 104 | 346 | 935 |
| | | | ethoxy)-ethoxy]-acetyl)₂-(γGlu)-CO-(CH₂)₁₆-CO₂H)AQ-αMe4Pal-Orn- | | | |
| | | | FIK(PEG₂₄-BEA-MalDap₂-Fc)-αMeY-LIEGGPSSGAPPPSGG-NH₂ | | | |
| 346 | | 802 | Y-Aib-QGT-αMeF(2F)-TSD-4Pal-αMeS-I-αMeL-LD-Orn-K(ϵKϵK-(γGlu)- | 103 | 347 | 935 |
| | | | CO-(CH₂)₁₆-CO₂H)AQ-αMe4Pal-Orn-FIK(PEG₂₄-BEA-MalDap₂-Fc)-αMeY- | | | |
| | | | LIEGGPSSGAPPPSGG-NH₂ | | | |
| 347 | | 803 | Y-Aib-QGT-αMeF(2F)-TSDY-αMeS-I-αMeL-LD-Orn-K(ϵKϵK-(γGlu)-CO- | 105 | 348 | 935 |
| | | | (CH₂)₁₆-CO₂H)AQ-αMe4Pal-Orn-FIK(PEG₂₄-BEA-MalDap₂-Fc)-αMeY- | | | |
| | | | LIEGGPSSGAPPPSGG-NH₂ | | | |
| 348 | | 804 | Y-Aib-QGT-αMeF(2F)-TSD-4Pal-αMeS-I-αMeL-LD-Orn-K(2-[2-(2-amino- | 106 | 349 | 935 |
| | | | ethoxy)-ethoxy]-acetyl)₂-(γGlu)-CO-(CH₂)₁₆-CO2H)AQ-αMe4Pal-Orn-FIE- | | | |
| | | | αMeY-LIEGGPSSGK(PEG₂₄-BEA-MalDap₂-Fc)-NH₂ | | | |
| 349 | | 805 | Y-Aib-QGT-αMeF(2F)-TSDY-αMeS-I-αMeL-LD-Orn-K(2-[2-(2-amino- | 107 | 350 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGPSSGK(PEG₂₄-BEA-MalDap₂-Fc)-NH₂ | | | |
| 350 | | 806 | Y-Aib-QGT-αMeF(2F)-TSD-4Pal-αMeS-I-αMeL-LD-Orn-K(ϵKϵK-(γGlu)- | 106 | 351 | 935 |
| | | | CO-(CH₂)₁₆-CO₂H)AQ-αMe4Pal-Orn-FIE-αMeY-LIEGGPSSGK(PEG₂₄- | | | |
| | | | BEA-MalDap₂-Fc)-NH₂ | | | |
| 351 | | 807 | Y-Aib-QGT-αMeF(2F)-TSDY-αMeS-I-αMeL-LD-Orn-KAQ-αMe4Pal-Orn- | 107 | 352 | 935 |
| | | | FIe-αMeY-LIEGGPSSGK(PEG₂₄-BEA-MalDap₂-Fc)-NH₂ | | | |
| 352 | | 808 | Y-Aib-QGT-αMeF(2F)-TSD-4Pal-αMeS-I-αMeL-LD-Orn-K(2-[2-(2-amino- | 99 | 339 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGGGK(PEG₂₄-BEA-MalDap2-Fc)-NH₂ | | | |
| 353 | | 809 | Y-Aib-QGT-αMeF(2F)-TSDY-αMeS-I-αMeL-LD-Orn-K(2-[2-(2-amino- | 100 | 340 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGK(PEG₂₄-BEA-MalDap₂-Fc)-NH₂ | | | |
| 354 | | 810 | Y-Aib-QGT-αMeF(2F)-TSD-4Pal-αMeS-I-αMeL-LD-Orn-K(ϵKϵK-(γGlu)- | 99 | 353 | 935 |
| | | | CO-(CH₂)₁₆-CO₂H)AQ-αMe4Pal-Orn-FIE-αMeY-LIEGGGK(PEG₂₄-BEA- | | | |
| | | | MalDap₂-Fc)-NH₂ | | | |
| 355 | | 811 | Y-Aib-QGT-αMeF(2F)-TSDY-αMeS-I-αMeL-LD-Orn-K(ϵKϵK-(γGlu)-CO- | 100 | 354 | 935 |
| | | | (CH₂)₁₆-CO₂H)AQ-αMe4Pal-Orn-FIe-αMeY-LIEGGGK(PEG₂₄-BEA- | | | |
| | | | MalDap₂-Fc)-NH₂ | | | |
| 356 | | 812 | Y-Aib-QGT-αMeF(2F)-TSD-4Pal-αMeS-I-αMeL-LD-Orn-K(2-12-(2-amino- | 99 | 339 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGGGGGGGQGGGGQGGGGQK(BEA-MalDap₂-Fc)-NH₂ | | | |
| 357 | | 813 | Y-Aib-QGT-αMeF(2F)-TSDY-αMeS-I-αMeL-LD-Orn-K(2-[2-(2-amino- | 100 | 340 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGGGGGQGGGGQGGGGQK(BEA-MalDap₂-Fc)-NH₂ | | | |
| 358 | | 814 | Y-Aib-QGT-αMeF(2F)-TSD-4Pal-αMeS-I-αMeL-LD-Orn-K(ϵKϵK-(γGlu)- | 96 | 355 | 935 |
| | | | CO-(CH₂)₁₆-CO2H)AQ-αMe4Pal-Orn-FIE-αMeY- | | | |
| | | | LIEGSPSSGAPPPSK(PEG₂₄-BEA-MalDap₂ Fc)-NH₂ | | | |
| 359 | | 815 | Y-Aib-QGT-αMeF(2F)-TSDY-αMeS-I-αMeL-LD-Orn-K(ϵKϵK-(γGlu)-CO- | 97 | 356 | 935 |
| | | | (CH₂)₁₆-CO₂H)AQ-αMe4Pal-Orn-FIe-αMeY-LIEGSPSSGAPPPSK(PEG₂₄- | | | |
| | | | BEA-MalDap₂ Fc)-NH₂ | | | |
| 360 | | 816 | Y-Aib-QGT-αMeF(2F)-TSD-4Pal-αMeS-I-αMeL-LD-Orn-K(2-12-(2-amino- | 108 | 357 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGK(PEG₂₄-BEA-MalDap₂ Fc)-NH₂ | | | |
| 361 | | 817 | Y-Aib-QGT-αMeF(2F)-TSD-4Pal-αMeS-I-αMeL-LD-Orn-K(2-12-(2-amino- | 109 | 358 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGGGGGSGGGGSGGGGSK(BEA-MalDap₂ Fc)-NH₂ | | | |
| 362 | | 818 | Y-Aib-QGT-αMeF(2F)-TSD-4Pal-αMeS-I-αMeL-LD-Orn-K(2-[2-(2-amino- | 109 | 358 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGGGGGSGGGGSGAPAPAPAPAPAPK(BEA-MalDap₂ Fc)- | | | |
| | | | NH₂ | | | |
| 363 | | 819 | Y-Aib-QGT-αMeF(2F)-TSD-4Pal-αMeS-I-αMeL-LD-Orn-K(2-[2-(2-amino- | 109 | 358 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGK(PEG₂₄-BEA-MalDap₂-Fc)-NH₂ | | | |
| 364 | | 820 | Y-Aib-QGT-αMeF(2F)-TSD-4Pal-αMeS-I-αMeL-LD-Orn-K(ϵKϵK-(γGlu)- | 95 | 359 | 935 |
| | | | CO-(CH₂)₁₆-CO₂H)AQ-αMe4Pal-Orn-FIe-αMeY- | | | |
| | | | LIEGSPSSGAPPPSK(PEG₂₄-BEA-MalDap₂-Fc)-NH₂ | | | |
| 365 | | 821 | Y-Aib-QGT-αMeF(2F)-TSD-4Pal-αMeS-I-αMeL-LD-Orn-K(2-12-(2-amino- | 110 | 360 | 935 |
| | | | ethoxy)-ethoxy]-acetyl)₂-(γGlu)-CO-(CH₂)₁₆-CO₂H)AQ-αMe4Pal-Orn-FIQ- | | | |
| | | | αMeY-LIEGSPSSGAPPPSK(PEG₂₄-BEA-MalDap₂-Fc)-NH₂ | | | |
| 366 | | 822 | Y-Aib-QGT-αMeF(2F)-TSD-4Pal-αMeS-I-αMeL-LD-Orn-K(ϵKϵK-(γGlu)- | 110 | 361 | 935 |
| | | | CO-(CH₂)₁₆-CO₂H)AQ-αMe4Pal-Orn-FIQ-αMeY- | | | |
| | | | LIEGSPSSGAPPPSK(PEG₂₄-BEA-MalDap₂-Fc)-NH₂ | | | |
| 367 | | 823 | Y-Aib-QGT-αMeF(2F)-TSDY-αMeS-I-αMeL-LD-Orn-K(2-12-(2-amino- | 111 | 362 | 935 |
| | | | ethoxy)-ethoxy]-acetyl)₂-(γGlu)-CO-(CH₂)₁₆-CO₂H)AQ-αMe4Pal-Orn-FIQ- | | | |
| | | | αMeY-LIEGSPSSGAPPPSK(PEG₂₄-BEA-MalDap₂-Fc)-NH₂ | | | |
| 368 | | 824 | Y-Aib-QGT-αMeF(2F)-TSDY-αMeS-I-αMeL-LD-Orn-K(ϵKϵK-(γGlu)-CO- | 111 | 363 | 935 |
| | | | (CH₂)₁₆-CO₂H)AQ-αMe4Pal-Orn-FIQ-αMeY-LIEGSPSSGAPPPSK(PEG₂₄- | | | |
| | | | BEA-MalDap₂-Fc)-NH₂ | | | |
| 369 | | 825 | Y-Aib-QGT-αMeF(2F)-TSDY-αMeS-I-αMeL-LD-Orn-K(2-[2-(2-amino- | 97 | 337 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(2-[2-(2-amino-ethoxy)-ethoxy]-acetyl)₆- | | | |
| | | | GPAPAPAPAPAPA-BEA(MalDap₂-Fc)-NH₂ | | | |
| 370 | | 826 | Y-Aib-QGT-αMeF(2F)-TSDY-αMeS-I-αMeL-LD-Orn-K(2-[2-(2-amino- | 112 | 364 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIE-Aib-SPSSGAPPPSK(PEG₂₄-BEA-MalDap₂-Fc)-NH₂ | | | |
| 371 | | 827 | Y-Aib-QGT-αMeF(2F)-TSDY-αMeS-I-αMeL-LD-Orn-K(2-[2-(2-amino- | 113 | 365 | 935 |
| | | | ethoxy)-ethoxy]-acetyl)₂-(γGlu)-CO-(CH₂)₁₆-CO₂H)AQ-αMe4Pal-EFIe- | | | |
| | | | αMeY-LIEGSPSSGAPPPSK(PEG₂₄-BEA-MalDap₂-Fc)-NH₂ | | | |
| 372 | | 828 | Y-Aib-QGT-αMeF(2F)-TSDY-αMeS-I-αMeL-LD-Orn-K(2-[2-(2-amino- | 114 | 366 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 373 | | 829 | Y-Aib-QGT-αMeF(2F)-TSDY-αMeS-I-αMeL-LD-Orn-K(2-[2-(2-amino- | 115 | 367 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂-Fc)-NH₂ | | | |
| 374 | | 830 | Y-Aib-QGT-αMeF(2F)-TSDY-αMeS-I-αMeL-LD-Orn-K(2-[2-(2-amino- | 116 | 368 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LLEGSPSSGAPPPSK(PEG₂₄-BEA-MalDap₂-Fc)-NH₂ | | | |
| 375 | | 831 | Y-Aib-QGT-αMeF(2F)-TSDY-αMeS-I-αMeL-LD-Orn-K(2-12-(2-amino- | 100 | 340 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGGGGGSGGGGSGGGGSK(BEA-MalDap₂-Fc)-NH₂ | | | |
| 376 | | 832 | Y-Aib-QGT-αMeF(2F)-TSD-4Pal-SI-αMeL-LD-Orn-K(2-[2-(2-amino- | 117 | 369 | 935 |
| | | | ethoxy)-ethoxy]-acetyl)₂-(γGlu)-CO-(CH₂)₁₆-CO₂H)AQ-αMe4Pal-Orn-FIQ- | | | |
| | | | αMeY-LIEGSPSSGAPPPSK(PEG₂₄-BEA-MalDap₂-Fc)-NH₂ | | | |
| 377 | | 833 | Y-Aib-QGT-αMeF(2F)-TSDY-αMeS-I-αMeL-LD-Orn-K(2-[2-(2-amino- | 118 | 370 | 935 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIE-Aib-GGGGGGSGGGGSGGGGSK(BEA-MalDap₂-Fc)-NH₂ | | | |
| 378 | | 834 | Y-Aib-QaT-αMeF(2F)-TSDY-αMeS-I-αMeL-LD-Orn-K(2-[2-(2-amino- | 119 | 371 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 379 | | 835 | Y-αMeS-QGT-αMeF(2F)-TSDY-αMeS-I-αMeL-LD-Orn-K(2-[2-(2-amino- | 120 | 372 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 380 | | 836 | Y-Iva-QGT-αMeF(2F)-TSDY-αMeS-I-αMeL-LD-Orn-K(2-[2-(2-amino- | 121 | 373 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 381 | | 837 | Y-Aib-HGT-αMeF(2F)-TSDY-αMeS-I-αMeL-LD-Orn-K(2-12-(2-amino- | 122 | 374 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH2 | | | |
| 382 | | 838 | H-Aib-HGT-αMeF(2F)-TSDY-αMeS-I-αMeL-LD-Orn-K(2-[2-(2-amino- | 123 | 375 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 383 | | 839 | H-Aib-HGT-αMeF(2F)-TSDY-αMeS-I-αMeL-LD-Orn-K(2-[2-(2-amino- | 124 | 376 | 935 |
| | | | ethoxy)-ethoxy]-acetyl)₂-(γGlu)-CO-(CH₂)₁₆-CO₂H)AQ-Aib-EFIQ-αMeY- | | | |
| | | | LIEGSPSSGAPPPSK(PEG₂₄-BEA-MalDap₂-Fc)-NH₂ | | | |
| 384 | | 840 | H-Aib-HGT-αMeF(2F)-TSD-4Pal-αMeS-I-αMeL-LD-Orn-K(2-[2-(2-amino- | 125 | 377 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 385 | | 841 | H-Aib-HGT-αMeF(2F)-TSD-4Pal-SI-αMeL-LD-Orn-K(2-12-(2-amino- | 126 | 378 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 386 | | 842 | Y-Aib-QGT-αMeF(2F)-TSDY-αMeS-I-αMeL-LD-Orn-K(2-[2-(2-amino- | 127 | 379 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIE-Aib-SPSSGAPPPSK(PEG₂₄-BEA-MalDap₂-Fc)-NH₂ | | | |
| 387 | 26 | 843 | Y-Aib-QGT-aMcF(2F)-TSDY-αMeS-I-αMeL-LD-Orn-K(2-[2-(2-amino- | 128 | 380 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 388 | | 844 | Y-Aib-QGT-αMeF(2F)-TSD-4Pal-αMeS-I-αMeL-LD-Orn-K(2-[2-(2-amino- | 129 | 381 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 389 | | 845 | Y-Aib-QGT-αMeF(2F)-TSD-4Pal-αMeS-I-αMeL-LD-Orn-K(2-[2-(2-amino- | 130 | 382 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 390 | | 846 | Y-Aib-QGT-αMeF(2F)-TSDY-αMeS-I-αMeL-LD-Orn-K(2-[2-(2-amino- | 114 | 383 | 935 |
| | | | ethoxy)-ethoxy]-acetyl)-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 391 | | 847 | Y-Aib-QGT-αMeF(2F)-TSDY-αMeS-I-αMeL-LD-Orn-K(2-[2-(2-amino- | 131 | 384 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIE-Aib-SPSSGAPPPSK(PEG₂₄-BEA-MalDap₂-Fc)-NH₂ | | | |
| 392 | | 848 | Y-Aib-QGT-αMeF(2F)-TSD-4Pal-αMeS-I-αMeL-LD-Orn-K(2-[2-(2-amino- | 132 | 385 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIE-Aib-SPSSGAPPPSK(PEG₂₄-BEA-MalDap₂-Fc)-NH₂ | | | |
| 393 | | 849 | Y-Aib-QGT-αMeF(2F)-TSD-4Pal-αMeS-I-αMeL-LD-Orn-K(2-[2-(2-amino- | 133 | 386 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIE-Aib-SPSSGAPPPSK(PEG₂₄-BEA-MalDap₂-Fc)-NH₂ | | | |
| 394 | | 850 | Y-Aib-QGT-αMeF(2F)-TSDY-αMeS-I-αMeL-LD-Orn-K(2-[2-(2-amino- | 127 | 387 | 935 |
| | | | ethoxy)-ethoxy]-acetyl)-(γGlu)-CO-(CH₂)₁₆-CO₂H)AQ-αMe4Pal-EFIQ- | | | |
| | | | αMeY-LIE-Aib-SPSSGAPPPSK(PEG₂₄-BEA-MalDap₂-Fc)-NH₂ | | | |
| 395 | | 851 | Y-Aib-QGT-αMeF(2F)-TSDY-αMeS-I-αMeL-LDEK(2-[2-(2-amino-ethoxy)- | 134 | 388 | 935 |
| | | | ethoxy]-acetyl)-(γGlu)-CO-(CH₂)₁₆-CO₂H)AQ-αMe4Pal-EFIQ-αMeY- | | | |
| | | | LLEGSPSSGAPPPSK(PEG₂₄-BEA-MalDap₂-Fc)-NH₂ | | | |
| 396 | | 852 | Y-Aib-QGT-αMeF(2F)-TSDY-αMeS-I-αMeL-LDEK(2-[2-(2-amino-ethoxy)- | 135 | 389 | 935 |
| | | | ethoxy]-acetyl)-(γGlu)-CO-(CH₂)₁₆-CO₂H)AQ-αMe4Pal-EFIQ-αMeY-LLE- | | | |
| | | | Aib-SPSSGAPPPSK(PEG₂₄-BEA-MalDap₂-Fc)-NH₂ | | | |
| 397 | | 853 | H-Aib-QGT-αMeF(2F)-TSDY-αMeS-I-αMeL-LD-Orn-K(2-[2-(2-amino- | 136 | 390 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap₂-Fc)-NH₂ | | | |
| 398 | | 854 | Y-Aib-QGT-αMeF(2F)-TSDY-αMeS-I-αMeL-LD-Orn-K(2-12-(2-amino- | 137 | 391 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LVEGSPSSGAPPPSK(PEG₂₄-BEA-MalDap₂-Fc)-NH₂ | | | |
| 399 | | 855 | Y-Aib-QGT-αMeF(2F)-TSDY-αMeS-I-αMeL-LD-Orn-K(2-[2-(2-amino- | 138 | 392 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGGGGGGGSGGGGSGGGGSK(BEA-MalDap₂-Fc)-NH₂ | | | |
| 400 | | 856 | Y-Aib-QGT-αMeF(2F)-TSDY-αMeS-I-αMeL-LD-Orn-K(2-12-(2-amino- | 114 | 366 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIEGSPSSGAPPPSK(2-[2-(2-amino-ethoxy)-ethoxy]-acetyl)₆-BEA- | | | |
| | | | MalDap₂-Fc)-NH₂ | | | |
| 401 | | 857 | H-Aib-HGT-αMeF(2F)-TSDY-αMeS-I-αMeL-LD-Orn-K(2-[2-(2-amino- | 139 | 393 | 935 |
| | | | ethoxy)-ethoxy]-acetyl)₂-(γGlu)-CO-(CH₂)₁₆-CO₂H)AQ-Aib-EFIQ-αMeY- | | | |
| | | | LLEGSPSSGAPPPSK(PEG₂₄-BEA-MalDap2-Fc)-NH₂ | | | |
| 402 | | 858 | H-Aib-HGT-αMeF(2F)-TSD-4Pal-SI-αMeL-LD-Orn-K(2-[2-(2-amino- | 140 | 394 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LLEGSPSSGAPPPSK(PEG₂₄-BEA-MalDap₂-Fc)-NH₂ | | | |
| 403 | 27 | 859 | Y-Aib-QGT-αMeF(2F)-TSDY-αMeS-I-αMeL-LD-Orn-K(2-[2-(2-amino- | 128 | 395 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 404 | | 860 | Y-Aib-QGT-αMeF(2F)-TSD-4Pal-αMeS-I-αMeL-LD-Orn-K(2-[2-(2-amino- | 129 | 396 | 935 |
| | | | ethoxy)-ethoxy]-acetyl)-(γGlu)-CO-(CH₂)₁₆-CO₂H)AQ-αMe4Pal-EFIQ- | | | |
| | | | αMeY-LIEGSPSSGAPPPSK(PEG₂₄-BEA-MalDap2-Fc)-NH₂ | | | |
| 405 | | 861 | Y-Aib-QGT-αMeF(2F)-TSDY-αMeS-I-αMeL-LD-Orn-K(2-[2-(2-amino- | 141 | 397 | 935 |
| | | | ethoxy)-ethoxy]-acetyl)₂-(γGlu)-CO-(CH₂)₁₆-CO₂H)AQ-αMe4Pal-DFIQ- | | | |
| | | | αMeY-LIEGSPSSGAPPPSK(2-[2-(2-amino-ethoxy)-ethoxy]-acetyl)₆-BEA- | | | |
| | | | MalDap₂-Fc)-NH₂ | | | |
| 406 | | 862 | Y-Aib-QGT-αMeF(2F)-TSDY-αMeS-I-αMeL-LD-Orn-K(2-[2-(2-amino- | 142 | 398 | 935 |
| | | | ethoxy)-ethoxy]-acetyl)₂₋(γGlu)-CO-(CH₂)₁₆-CO₂H)AQ-αMe4Pal-Orn-FIE- | | | |
| | | | αMeY-LIA-Aib-SPSSGAPPPSK(2-[2-(2-amino-ethoxy)-ethoxy]-acetyl)₆- | | | |
| | | | BEA-MalDap₂-Fc)-NH₂ | | | |
| 407 | | 863 | Y-Aib-QGT-αMeF(2F)-TSDY-αMeS-I-αMeL-LD-Orn-K(2-[2-(2-amino- | 116 | 368 | 935 |
| | | | ethoxy)-ethoxy]-acetyl)2-(γGlu)-CO-(CH2)16-CO2H)AQ-αMe4Pal-EFIQ- | | | |
| | | | αMeY-LLEGSPSSGAPPPS-K(2-[2-(2-amino-ethoxy)-ethoxy]-acetyl)₆-BEA- | | | |
| | | | MalDap₂-Fc)-NH₂ | | | |
| 408 | | 864 | Y-Aib-QGT-αMeF(2F)-TSDY-αMeS-I-αMeL-LD-Orn-K(2-[2-(2-amino- | 143 | 399 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LLE-Aib-SPSSGAPPPSK(2-[2-(2-amino-ethoxy)-ethoxy]-acetyl)₆- | | | |
| | | | BEA-MalDap₂-Fc)-NH₂ | | | |
| 409 | | 865 | H-Aib-QGT-αMeF(2F)-TSDY-αMeS-I-αMeL-LD-Orn-KAQ-αMe4Pal-EFIE- | 144 | 400 | 935 |
| | | | αMeY-LIEGSPSSGAPPPSK(PEG₂₄-BEA-MalDap₂-Fc)-NH₂ | | | |
| 410 | | 866 | H-Aib-QGT-αMeF(2F)-TSDY-αMeS-I-αMeL-LD-Orn-K(2-[2-(2-amino- | 145 | 401 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIE-Aib-SPSSGAPPPSK(PEG₂₄-BEA-MalDap₂-Fc)-NH₂ | | | |
| 411 | | 867 | H-Aib-QGT-αMeF(2F)-TSDY-αMeS-I-αMeL-LD-Orn-K(2-[2-(2-amino- | 146 | 402 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LLEGSPSSGAPPPSK(PEG₂₄-BEA-MalDap₂-Fc)-NH₂ | | | |
| 412 | | 868 | H-Aib-QGT-αMeF(2F)-TSDY-αMeS-I-αMeL-LD-Orn-K(2-[2-(2-amino- | 147 | 403 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LLE-Aib-SPSSGAPPPSK(PEG₂₄-BEA-MalDap₂-Fc)-NH₂ | | | |
| 413 | 28 | 869 | H-Aib-QGT-αMeF(2F)-TSDY-αMeS-I-αMeL-LD-Orn-K(2-[2-(2-amino- | 144 | 404 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG24-BEA-MalDap₂-Fc)-NH₂ | | | |
| 414 | | 870 | H-Aib-QGT-αMeF(2F)-TSDY-αMeS-I-αMeL-LD-Orn-K(2-[2-(2-amino- | 145 | 405 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIE-Aib-SPSSGAPPPSK(PEG₂₄-BEA-MalDap₂-Fc)-NH₂ | | | |
| 415 | | 871 | H-Aib-QGT-αMeF(2F)-TSDY-αMeS-I-αMeL-LD-Orn-K(2-12-(2-amino- | 146 | 406 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LLEGSPSSGAPPPSK(PEG₂₄-BEA-MalDap₂-Fc)-NH₂ | | | |
| 416 | | 872 | H-Aib-QGT-αMeF(2F)-TSDY-αMeS-I-αMeL-LD-Orn-K(2-12-(2-amino- | 148 | 407 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LIE-Aib-SPSSGAPPPSK(PEG₂₄-BEA-MalDap₂-Fc)-NH₂ | | | |
| 417 | | 873 | H-Aib-QGT-αMeF(2F)-TSDY-αMeS-I-αMeL-LD-Orn-K(2-12-(2-amino- | 149 | 408 | 935 |
| | | | ethoxy)-ethoxy]-acetyl)₂-(γGlu)-CO-(CH₂)₁₆-CO₂H)AQ-αMe4Pal-EFIQ- | | | |
| | | | αMeY-LLE-Aib-SPSSGAPPPSK(PEG₂₄-BEA-MalDap₂-Fc)-NH₂ | | | |
| 418 | | 874 | H-Aib-QGT-αMeF(2F)-TSDY-αMeS-I-αMeL-LD-Orn-K(2-[2-(2-amino- | 148 | 409 | 935 |
| | | | ethoxy)-ethoxy]-acetyl)-(γGlu)-CO-(CH₂)₁₆-CO₂H)AQ-αMe4Pal-EFIQ- | | | |
| | | | αMeY-LIE-Aib-SPSSGAPPPSK(PEG24-BEA-MalDap₂-Fc)-NH₂ | | | |
| 419 | | 875 | H-Aib-QGT-αMeF(2F)-TSDY-αMeS-I-αMeL-LD-Orn-K(2-[2-(2-amino- | 149 | 410 | 935 |
| | | | ethoxy)-ethoxy]-acetyl)-(γGlu)-CO-(CH₂)₁₆-CO₂H)AQ-αMe4Pal-EFIQ- | | | |
| | | | αMeY-LLE-Aib-SPSSGAPPPSK(PEG24-BEA-MalDap₂-Fc)-NH₂ | | | |
| 420 | | 876 | Y-αMeS-QGT-αMeF(2F)-TSDY-αMeS-I-αMeL-LD-Orn-K(2-[2-(2-amino- | 150 | 411 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG24-BEA-MalDap₂-Fc)-NH₂ | | | |
| 421 | | 877 | Y-Iva-QGT-αMeF(2F)-TSDY-αMeS-I-αMeL-LD-Orn-K(2-[2-(2-amino- | 151 | 412 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG24-BEA-MalDap₂-Fc)-NH₂ | | | |
| 422 | | 878 | Y-Ac4c-QGT-αMeF(2F)-TSDY-αMeS-I-αMeL-LD-Orn-K(2-[2-(2-amino- | 152 | 413 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 423 | | 879 | H-Aib-QGT-αMeF(2F)-TSDY-αMeS-I-αMeL-LD-Orn-K(2-[2-(2-amino- | 153 | 414 | 935 |
| | | | ethoxy)-ethoxy]-acetyl)-(γGlu)-CO-(CH₂)₁₆-CO₂H)AQ-αMe4Pal-Orn- | | | |
| | | | FIK(PEG₂₄-BEA-MalDap₂-Fc)-αMeY-LLEGSPSSGAPPPS-NH₂ | | | |
| 424 | | 880 | H-Aib-QGT-αMeF(2F)-TSDY-αMeS-I-αMeL-LD-Orn-K(2-12-(2-amino- | 154 | 415 | 935 |
| | | | ethoxy)-ethoxy]-acetyl)-(γGlu)-CO-(CH₂)₁₆-CO₂H)AQ-αMe4Pal- | | | |
| | | | EFIK(PEG₂₄-BEA-MalDap₂-Fc)-αMeY-LLEGSPSSGAPPPS-NH₂ | | | |
| 425 | | 881 | H-Aib-QGT-αMeF(2F)-TSDY-αMeS-I-αMeL-LD-Orn-K(2-[2-(2-amino- | 155 | 416 | 935 |
| | | | ethoxy)-ethoxy]-acetyl)-(γGlu)-CO-(CH₂)1₆-CO₂H)AQ-αMe4Pal-Orn-FIe- | | | |
| | | | αMeY-LIEGSPSSGAPPPSK(PEG₂₄-BEA-MalDap₂-Fc)-NH₂ | | | |
| 426 | | 882 | H-Aib-QGT-αMeF(2F)-TSDY-αMeS-I-αMeL-LD-Orn-K(2-12-(2-amino- | 156 | 417 | 935 |
| | | | ethoxy)-ethoxy]-acetyl)-(γGlu)-CO-(CH₂)₁₆-CO₂H)AQ-αMe4Pal-Orn-FIe- | | | |
| | | | αMeY-LLEGSPSSGAPPPSK(PEG₂₄-BEA-MalDap₂-Fc)-NH₂ | | | |
| 427 | | 883 | H-Aib-QGT-αMeF(2F)-TSDY-αMeS-I-αMeL-LD-Orn-K(2-[2-(2-amino- | 157 | 418 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 428 | | 884 | H-Aib-QGT-αMeF(2F)-TSDY-αMeS-I-αMeL-LD-Orn-K(2-[2-(2-amino- | 158 | 419 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LLEGSPSSGAPPPSK(PEG₂₄-BEA-MalDap₂-Fc)-NH₂ | | | |
| 429 | | 885 | Y-Aib-QGT-αMeF(2F)-TSDY-αMeS-I-αMeL-LD-Orn-K(2-[2-(2-amino- | 159 | 420 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LLEGSPSSGAPPPSK(PEG₂₄-BEA-MalDap₂-Fc)-NH₂ | | | |
| 430 | | 886 | Y-Aib-QGT-αMeF(2F)-TSDY-αMeS-I-αMeL-LD-Orn-K(2-[2-(2-amino- | 160 | 421 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LLEGSPSSGAPPPSK(PEG₂₄-BEA-MalDap₂-Fc)-NH₂ | | | |
| 431 | | 887 | Y-Aib-QGT-αMeF(2F)-TSDY-αMeS-I-αMeL-LD-Orn-K(2-[2-(2-amino- | 161 | 422 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LLEGSPSSGAPPPSK(PEG₂₄-BEA-MalDap₂-Fc)-NH₂ | | | |
| 432 | | 888 | Y-Aib-QGT-αMeF(2F)-TSDY-αMeS-I-αMeL-LD-Orn-K(2-[2-(2-amino- | 162 | 423 | 935 |
| | | | ethoxy)-ethoxy]-acetyl)-(γGlu)-CO-(CH₂)₁₆-CO₂H)AQ-αMe4Pal-Orn-FIe- | | | |
| | | | αMeY-LLEGSPSSGAPPPSK(PEG₂₄-BEA-MalDap₂-Fc)-NH₂ | | | |
| 433 | 29 | 889 | Y-Aib-QGT-αMeF(2F)-TSDY-αMeS-I-αMeL-LD-Orn-K(2-[2-(2-amino- | 113 | 424 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIEGSPSSGAPPPSK(PEG₂₄-BEA-MalDap₂-Fc)-NH₂ | | | |
| 434 | | 890 | Y-Aib-QGT-αMeF(2F)-TSDY-αMeS-I-aML-LD-Orn-K(2-[2-(2-amino- | 98 | 425 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 435 | 30 | 891 | Y-Aib-QGT-αMeF(2F)-TSDY-αMeS-I-αMeL-LD-Orn-K(2-12-(2-amino- | 97 | 426 | 935 |
| | | | ethoxy)-ethoxy]-acetyl)-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc)-NH₂ | | | |
| 436 | | 892 | Y-Aib-QGT-αMeF(2F)-TSDY-αMeS-I-αMeL-LD-Orn-K(2-[2-(2-amino- | 163 | 427 | 935 |
| | | | ethoxy)-ethoxy]-acetyl)-(γGlu)-CO-(CH₂)₁₆-CO₂H)AQ-Aib-QFIE-αMeY- | | | |
| | | | LLEGSPSSGAPPPSK(PEG₂₄-BEA-MalDap₂-Fc)-NH₂ | | | |
| 437 | | 893 | Y-Aib-QGT-αMeF(2F)-TSDY-αMeS-I-αMeL-LD-Orn-K(2-[2-(2-amino- | 164 | 428 | 935 |
| | | | ethoxy)-ethoxy]-acetyl)-(γGlu)-CO-(CH₂)₁₆-CO₂H)AQ-Aib-Orn-FIe-αMeY- | | | |
| | | | LLEGSPSSGAPPPSK(PEG₂₄-BEA-MalDap₂-Fc)-NH₂ | | | |
| 438 | | 894 | Y-Aib-QGT-αMeF(2F)-TSDY-αMeS-I-αMeL-LD-Orn-K(2-12-(2-amino- | 165 | 429 | 935 |
| | | | ethoxy)-ethoxy]-acetyl)-(γGlu)-CO-(CH₂)₁₆-CO₂H)AQ-Aib-Orn-FIE-αMeY- | | | |
| | | | LLEGSPSSGAPPPSK(PEG₂₄-BEA-MalDap2-Fc)-NH₂ | | | |
| 439 | | 895 | Y-Aib-QGT-αMeF(2F)-TSDY-αMeS-I-αMeL-LD-Orn-K(2-[2-(2-amino- | 166 | 430 | 935 |
| | | | ethoxy)-ethoxy]-acetyl)-(γGlu)-CO-(CH₂)₁₆-CO₂H)AQ-Aib-Orn-FIq-αMeY- | | | |
| | | | LLEGSPSSGAPPPSK(PEG₂₄-BEA-MalDap₂-Fc)-NH₂ | | | |
| 440 | | 896 | Y-Aib-QGT-αMeF(2F)-TSDY-αMeS-I-αMeL-LD-Orn-K(2-[2-(2-amino- | 167 | 431 | 935 |
| | | | ethoxy)-ethoxy]-acetyl)-(γGlu)-CO-(CH₂)₁₆-CO₂H)AQ-Aib-Orn-FIQ-αMeY- | | | |
| | | | LLEGSPSSGAPPPSK(PEG₂₄-BEA-MalDap₂-Fc)-NH₂ | | | |
| 441 | | 897 | Y-Aib-QGT-αMeF(2F)-TSDY-αMeS-I-αMeL-LD-Orn-K(2-12-(2-amino- | 168 | 432 | 935 |
| | | | ethoxy)-ethoxy]-acetyl)-(γGlu)-CO-(CH₂)₁₆-CO₂H)AQ-Aib-EFIe-αMeY- | | | |
| | | | LLEGSPSSGAPPPSK(PEG₂₄-BEA-MalDap₂-Fc)-NH₂ | | | |
| 442 | | 898 | Y-Aib-QGT-αMeF(2F)-TSDY-αMeS-I-αMeL-LD-Orn-K(2-[2-(2-amino- | 169 | 433 | 935 |
| | | | ethoxy)-ethoxy]-acetyl)-(γGlu)-CO-(CH₂)₁₆-CO₂H)AQ-Aib-EFIE-αMeY- | | | |
| | | | LLEGSPSSGAPPPSK(PEG₂₄-BEA-MalDap₂-Fc)-NH₂ | | | |
| 443 | | 899 | Y-Aib-QGT-αMeF(2F)-TSDY-αMeS-I-αMeL-LD-Orn-K(2-[2-(2-amino- | 170 | 434 | 935 |
| | | | ethoxy)-ethoxy]-acetyl)-(γGlu)-CO-(CH₂)₁₆-CO₂H)AQ-Aib-EFIq-αMeY- | | | |
| | | | LLEGSPSSGAPPPSK(PEG₂₄-BEA-MalDap₂-Fc)-NH₂ | | | |
| 444 | 31 | 900 | H-Ac4c-QGT-αMeF(2F)-TSDY-αMeS-I-αMeL-LD-Orn-K(2-[2-(2-amino- | 171 | 435 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 445 | | 901 | YaQGT-αMeF(2F)-TSDY-αMeS-I-αMeL-LD-Orn-K(2-[2-(2-amino-ethoxy)- | 172 | 436 | 935 |
| | | | ethoxy]-acetyl)-(γGlu)-CO-(CH₂)₁₆-CO₂H)AQ-αMe4Pal-EFIE-αMeY- | | | |
| | | | LIEGSPSSGAPPPSK(PEG₂₄-BEA-MalDap₂-Fc)-NH₂ | | | |
| 446 | | 902 | YaQGT-αMeF(2F)-TSDY-αMeS-I-αMeL-LD-Orn-K(2-[2-(2-amino-ethoxy)- | 173 | 437 | 935 |
| | | | ethoxy]-acetyl)-(γGlu)-CO-(CH₂)₁₆-CO₂H)AQ-αMe4Pal-Orn-FIE-αMeY- | | | |
| | | | LIEGSPSSGAPPPSK(PEG₂₄-BEA-MalDap₂-Fc)-NH₂ | | | |
| 447 | | 903 | YaQGT-αMeF(2F)-TSDY-αMeS-I-αMeL-LD-Orn-K(2-[2-(2-amino-ethoxy)- | 174 | 438 | 935 |
| | | | ethoxy]-acetyl)-(γGlu)-CO-(CH₂)₁₆-CO₂H)AQ-αMe4Pal-Orn-FIe-αMeY- | | | |
| | | | LIEGSPSSGAPPPSK(PEG₂₄-BEA-MalDap₂-Fc)-NH₂ | | | |
| 448 | | 904 | YaQGT-αMeF(2F)-TSDY-αMeS-I-αMeL-LD-Orn-K(2-[2-(2-amino-ethoxy)- | 175 | 439 | 935 |
| | | | ethoxy]-acetyl)-(γGlu)-CO-(CH₂)₁₆-CO₂H)AQ-αMe4Pal-EFIE-αMeY- | | | |
| | | | LLEGSPSSGAPPPSK(PEG₂₄-BEA-MalDap₂-Fc)-NH₂ | | | |
| 449 | | 905 | YaQGT-αMeF(2F)-TSDY-αMeS-I-αMeL-LD-Orn-K(2-[2-(2-amino-ethoxy)- | 176 | 440 | 935 |
| | | | ethoxy]-acetyl)-(γGlu)-CO-(CH₂)₁₆-CO₂H)AQ-αMe4Pal-EFIe-αMeY- | | | |
| | | | LLEGSPSSGAPPPSK(PEG₂₄-BEA-MalDap₂-Fc)-NH₂ | | | |
| 450 | | 906 | YaQGT-αMeF(2F)-TSDY-αMeS-I-αMeL-LD-Orn-K(2-[2-(2-amino-ethoxy)- | 177 | 441 | 935 |
| | | | ethoxy]-acetyl)-(γGlu)-CO-(CH₂)₁₆-CO₂H)AQ-αMe4Pal-Orn-FIE-αMeY- | | | |
| | | | LLEGSPSSGAPPPSK(PEG₂₄-BEA-MalDap₂-Fc)-NH₂ | | | |
| 451 | | 907 | YaQGT-αMeF(2F)-TSDY-αMeS-I-αMeL-LD-Orn-K(2-[2-(2-amino-ethoxy)- | 178 | 442 | 935 |
| | | | ethoxy]-acetyl)-(γGlu)-CO-(CH₂)₁₆-CO₂H)AQ-Aib-Orn-FIq-αMeY- | | | |
| | | | LLEGSPSSGAPPPSK(PEG₂₄-BEA-MalDap₂-Fc)-NH₂ | | | |
| 452 | | 908 | YaQGT-αMeF(2F)-TSDY-αMeS-I-αMeL-LD-Orn-K(2-[2-(2-amino-ethoxy)- | 179 | 443 | 935 |
| | | | ethoxy]-acetyl)-(γGlu)-CO-(CH₂)₁₆-CO₂H)AQ-Aib-Orn-FIQ-αMeY- | | | |
| | | | LLEGSPSSGAPPPSK(PEG₂₄-BEA-MalDap₂-Fc)-NH₂ | | | |
| 453 | | 909 | YaQGT-aMcF(2F)-TSDY-αMeS-I-αMeL-LD-Orn-K(2-[2-(2-amino-ethoxy)- | 180 | 444 | 935 |
| | | | ethoxy]-acetyl)-(γGlu)-CO-(CH₂)₁₆-CO₂H)AQ-Aib-Orn-FIE-αMeY- | | | |
| | | | LIEGSPSSGAPPPSK(PEG₂₄-BEA-MalDap₂-Fc)-NH₂ | | | |
| 454 | | 910 | Y-Aib-QGT-αMeF(2F)-TSD-4Pal-SI-αMeL-LD-Orn-K(2-[2-(2-amino- | 94 | 445 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 455 | | 911 | Y-Aib-QGT-αMeF(2F)-TSD-4Pal-αMeS-I-αMeL-LD-Orn-K(2-[2-(2-amino- | 95 | 446 | 935 |
| | | | ethoxy)-ethoxy]-acetyl)-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc)-NH₂ | | | |
| 456 | 35 | 912 | Y-Aib-QGT-αMeF(2F)-TSD-4Pal-αMeS-I-αMeL-LD-Orn-K(2-[2-(2-amino- | 130 | 447 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 457 | | 913 | Y-Aib-QGT-αMeF(2F)-TSD-4Pal-SI-αMeL-LD-Orn-K(2-[2-(2-amino- | 181 | 448 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 458 | | 914 | Y-Aib-QGT-αMeF(2F)-TSDY-αMeS-I-αMeL-LD-Orn-K(2-[2-(2-amino- | 182 | 449 | 935 |
| | | | ethoxy)-ethoxy]-acetyl)-(γGlu)-CO-(CH₂)₁₆-CO₂H)AQ-Aib-Orn-FIE-αMeY- | | | |
| | | | LIEGSPSSGAPPPSK(PEG₂₄-BEA-MalDap₂-Fc)-NH₂ | | | |
| 459 | 38 | 915 | Y-Aib-QGT-αMeF(2F)-TSD-4Pal-αMeS-I-αMeL-LD-Orn-K(2-[2-(2-amino- | 183 | 450 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIEGSPSSGAPPPSK(PEG₂₄-BEA-MalDap₂-Fc)-NH₂ | | | |
| 460 | | 916 | YaQGT-αMeF(2F)-TSDY-αMeS-I-αMeL-LD-Orn-K(2-[2-(2-amino-ethoxy)- | 173 | 451 | 935 |
| | | | ethoxy]-acetyl)₂-(γGlu)-CO-(CH2)16-CO2H)AQ-αMe4Pal-Orn-FIE-αMeY- | | | |
| | | | LIEGSPSSGAPPPSK(PEG24-BEA-MalDap2-Fc)-NH2 | | | |
| 461 | | 917 | YaQGT-αMeF(2F)-TSDY-αMeS-I-αMeL-LD-Orn-K(2-[2-(2-amino-ethoxy)- | 177 | 452 | 935 |
| | | | ethoxy]-acetyl)₂-(γGlu)-CO-(CH₂)₁₆-CO₂H)AQ-αMe4Pal-Orn-FIE-αMeY- | | | |
| | | | LLEGSPSSGAPPPSK(PEG₂₄-BEA-MalDap₂-Fc)-NH₂ | | | |
| 462 | | 918 | Y-Aib-QGT-αMeF(2F)-TSDY-αMeS-I-αMeL-LD-Orn-K(2-[2-(2-amino- | 184 | 453 | 935 |
| | | | ethoxy)-ethoxy]-acetyl)-(γGlu)-CO-(CH₂)₁₆-CO₂H)AQ-Aib-Orn-FIe-αMeY- | | | |
| | | | LIEGSPSSGAPPPSK(PEG₂₄-BEA-MalDap₂-Fc)-NH₂ | | | |
| 463 | | 919 | NMeY-Aib-QGT-αMeF(2F)-TSDY-αMeS-I-αMeL-LD-Orn-K(2-[2-(2-amino- | 185 | 454 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 464 | | 920 | Y-Aib-QGT-αMeF(2F)-TSDY-αMeS-I-αMeL-LD-Orn-K(2-12-(2-amino- | 97 | 455 | 935 |
| | | | ethoxy)-ethoxy]-acetyl)₂-(γGlu)-CO-(CH₂)₁₈-CO₂H)AQ-αMe4Pal-Orn-FIe- | | | |
| | | | αMeY-LIEGSPSSGAPPPSK(PEG24-BEA-MalDap₂-Fc)-NH₂ | | | |
| 465 | | 921 | Y-Aib-QGT-αMeF(2F)-TSDY-αMeS-I-αMeL-LD-Orn-K(2-12-(2-amino- | 111 | 456 | 935 |
| | | | ethoxy)-ethoxy]-acetyl)₂-(γGlu)-CO-(CH₂)₁₈-CO₂H)AQ-αMe4Pal-Orn-FIQ- | | | |
| | | | αMeY-LIEGSPSSGAPPPSK(PEG₂₄-BEA-MalDap₂-Fc)-NH₂ | | | |
| 466 | | 922 | Y-Aib-QGT-αMeF(2F)-TSD-4Pal-αMeS-I-αMeL-LD-Orn-K(2-[2-(2-amino- | 95 | 335 | 936 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc KRQ)-NH₂ | | | |
| 467 | | 923 | Y-Aib-QGT-αMeF(2F)-TSDY-αMeS-I-αMeL-LD-Orn-K(2-12-(2-amino- | 97 | 337 | 936 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc KRQ)-NH₂ | | | |
| 468 | | 924 | Y-Aib-QGT-αMeF(2F)-TSD-4Pal-αMeS-I-αMeL-LD-Orn-K(2-12-(2-amino- | 99 | 339 | 936 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGGGGGGGSGGGGSGAPAPAPAPAPAPK(BEA-MalDap₂-Fc | | | |
| | | | KRQ)-NH₂ | | | |
| 469 | | 925 | Y-Aib-QGT-αMeF(2F)-TSDY-αMeS-I-αMeL-LD-Orn-K(2-[2-(2-amino- | 100 | 340 | 936 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGGGGGGGSGGGGSGAPAPAPAPAPAPK(BEA-MalDap₂-Fc | | | |
| | | | KRQ)-NH₂ | | | |
| 470 | | 926 | Y-Aib-QGT-αMeF(2F)-TSDY-αMeS-I-αMeL-LD-Orn-K(2-[2-(2-amino- | 97 | 337 | 936 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(2-[2-(2-amino-ethoxy)-ethoxy]-acetyl)₆- | | | |
| | | | GPAPAPAPAPAPA-BEA(MalDap₂-Fc KRQ)-NH₂ | | | |
| 471 | | 927 | Y-Aib-QGT-αMeF(2F)-TSDY-αMeS-I-αMeL-LD-Orn-K(2-[2-(2-amino- | 97 | 337 | 941 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(PEG₂₄-BEA-MalDap₂-Fc IG KRQ)-NH₂ | | | |
| 472 | | 928 | Y-Aib-QGT-αMeF(2F)-TSDY-αMeS-I-αMeL-LD-Orn-K(2-12-(2-amino- | 112 | 364 | 941 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIE-Aib-SPSSGAPPPSK(PEG₂₄-BEA-MalDap₂-Fc IG KRQ)-NH₂ | | | |
| 473 | | 929 | Y-Aib-QGT-αMeF(2F)-TSDY-αMeS-I-αMeL-LD-Orn-K(2-12-(2-amino- | 97 | 337 | 941 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIEGSPSSGAPPPSK(2-[2-(2-amino-ethoxy)-ethoxy]-acetyl)₆-BEA- | | | |
| | | | MalDap₂-Fc IG KRQ)-NH₂ | | | |
| 474 | | 930 | Y-Aib-QGT-αMeF(2F)-TSDY-αMeS-I-αMeL-LD-Orn-K(2-[2-(2-amino- | 118 | 370 | 941 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIE-Aib-GGGGGGSGGGGSGAPAPAPAPAPAPK(BEA-MalDap₂- | | | |
| | | | Fc IG KRQ)-NH₂ | | | |
| 475 | | 931 | Y-Aib-QGT-αMeF(2F)-TSDY-αMeS-I-αMeL-LD-Orn-K(2-[2-(2-amino- | 112 | 364 | 941 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIE-Aib-SPSSGAPPPSK(2-[2-(2-amino-ethoxy)-ethoxy]-acetyl)₆- | | | |
| | | | GPAPAPAPAPAPA-BEA(MalDap₂-Fc IG KRQ)-NH₂ | | | |
| 476 | | 932 | Y-Aib-QGT-αMeF(2F)-TSDY-αMeS-I-αMeL-LD-Orn-K(2-[2-(2-amino- | 112 | 364 | 941 |
| | | | ethoxy)-ethoxy]-acetyl)₂-(γGlu)-CO-(CH₂)₁₆-CO₂H)AQ-αMe4Pal-Orn-FIe- | | | |
| | | | αMeY-LIE-Aib-SPSSGAPPPSK(2-[2-(2-amino-ethoxy)-ethoxy]-acetyl)₆- | | | |
| | | | BEA-MalDap₂-Fc IG KRQ)-NH₂ | | | |
| 477 | | 933 | Y-Aib-QGT-αMeF(2F)-TSDY-αMeS-I-αMeL-LD-Orn-K(2-[2-(2-amino- | 112 | 457 | 941 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIE-Aib-SPSSGAPPPSK(PEG₂₄-BEA-MalDap₂-Fc IG KRQ)-NH₂ | | | |
| 478 | | 934 | Y-Aib-QGT-αMeF(2F)-TSDY-αMeS-I-αMeL-LD-Orn-K(2-[2-(2-amino- | 186 | 458 | 941 |
| | | | ethoxy)-ethoxy]-acetyl)₂-(γGlu)-CO-(CH₂)₁₆-CO₂H)AQ-αMe4Pal-Orn-FIQ- | | | |
| | | | αMeY-LIE-Aib-SPSSGAPPPSK(PEG₂₄-BEA-MalDap₂-Fc IG KRQ)-NH₂ | | | |
| 479 | 21 | 1099 | Y-Aib-EGT-αMeF(2F)-TSD-4Pal-SI-αMeL-LDEK(2-[2-(2-amino-ethoxy)- | 1 | 187 | 935 |
| | | | ethoxy]-acetyl)₂-(γGlu)-CO-(CH₂)₁₆-CO₂H)AQ-Aib-EFIe-αMeY- | | | |
| | | | LIEGSPSSGAPPPSK(PEG₂₄-dCAP-Fc)-NH₂ | | | |
| 483 | 32 | 1100 | Y-Aib-EGT-αMeF(2F)-TSDY-Aib-I-αMeL-LD-Orn-K(2-[2-(2-amino- | 59 | 264 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-MSPT2-Fc)-NH₂ | | | |
| 484 | 33 | 1101 | Y-Aib-QGT-αMeF(2F)-TSDY-αMeS-I-αMeL-LD-Orn-K(2-[2-(2-amino- | 128 | 380 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEGSPSSGAPPPSK(PEG₂₄-BEA-MSPT2-Fc)-NH₂ | | | |
| 485 | 34 | 1102 | Y-Aib-QGT-aMcF(2F)-TSD-4Pal-αMeS-I-αMeL-LD-Orn-K(2-[2-(2-amino- | 130 | 447 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-MSPT2-Fc)-NH₂ | | | |
| 486 | 36 | 1103 | Y-Aib-QGT-αMeF(2F)-TSDY-αMeS-I-αMeL-LD-Orn-K(2-[2-(2-amino- | 113 | 424 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIEGSPSSGAPPPSK(PEG₂₄-BEA-MSPT2-Fc)-NH₂ | | | |
| 487 | 37 | 1104 | Y-Aib-QGT-αMeF(2F)-TSD-4Pal-αMeS-I-αMeL-LD-Orn-K(2-[2-(2-amino- | 183 | 450 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIEGSPSSGAPPPSK(PEG₂₄-BEA-MSPT2-Fc)-NH₂ | | | |
| 488 | | 1105 | Y-Aib-EGT-αMeF(2F)-TSD-4Pal-Aib-I-αMeL-LD-Orn-QAQ-Aib-EFIe- | 187 | N/A | 935 |
| | | | αMeY-LIEGGG(GGGGS)₃-K(BEA-MalDap2-Fc)-NH₂ | | | |
| 489 | | 1106 | Y-Aib-EGT-αMeF(2F)-TSD-Y-Aib-I-αMeL-LD-Orn-K(2-12-(2-amino- | 188 | 502 | 935 |
| | | | ethoxy)-ethoxy]-acetyl)₂-(γGlu)-CO-(CH₂)₁₆-CO2H)AQ-Aib-Orn-FIE-αMeY- | | | |
| | | | LIEGSPSSGAPPPSK(PEG₂₄-BEA-MalDap₂-Fc)-NH₂ | | | |
| 490 | | 1107 | Y-Aib-EGT-αMeF(2F)-TSDY-Aib-I-αMeL-LD-Orn-K(2-[2-(2-amino- | 189 | 503 | 935 |
| | | | ethoxy)-ethoxy]-acetyl)₂-(γGlu)-CO-(CH₂)₁₆-CO₂H)AQ-Aib-EFIE-αMeY- | | | |
| | | | LIAGGGK(PEG₂₄-BEA-MSPT2-Fc)-NH₂ | | | |
| 491 | | 1108 | Y-Aib-EGT-αMeF(2F)-TSDY-Aib-I-αMeL-LD-Orn-K(2-[2-(2-amino- | 190 | 504 | 935 |
| | | | ethoxy)-ethoxy]-acetyl)₂-(γGlu)-CO-(CH₂)₁₆-CO₂H)AQ-Aib-EFIE-αMeY- | | | |
| | | | LIE-Aib-GGK(PEG₂₄-BEA-MSPT2-Fc)-NH₂ | | | |
| 492 | | 1109 | Y-Aib-EGT-αMeF(2F)-TSDY-Aib-I-αMeL-LD-Orn-K(2-[2-(2-amino- | 59 | 505 | 935 |
| | | | ethoxy)-ethoxy]-acetyl)₂-(γGlu)-CO-(CH₂)₁₄-CO₂H)AQ-Aib-EFIE-αMeY- | | | |
| | | | LIEGSPSSGAPPPSK(PEG₂₄-BEA-MalDap₂-Fc)-NH₂ | | | |
| 493 | 39 | 1110 | Y-Aib-EGT-αMeF(2F)-TSDY-Aib-I-αMeL-LD-Orn-K(2-[2-(2-amino- | 59 | 307 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-B-MalDap₂-Fc)-NH₂ | | | |
| 494 | | 1111 | Y-Aib-QGT-αMeF(2F)-TSDY-αMeS-I-αMeL-LD-Orn-K(2-[2-(2-amino- | 191 | 506 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LVEGSPSSGAPPPSK(PEG₂₄-BEA-MalDap₂-Fc)-NH₂ | | | |
| 495 | | 1112 | Y-Aib-QGT-αMeF(2F)-TSD-4Pal-αMeS-I-αMeL-LD-Orn-K(2-[2-(2-amino- | 96 | 507 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 496 | | 1113 | Y-Aib-QGT-αMeF(2F)-TSD-4Pal-αMeS-I-αMeL-LD-Orn-K(2-[2-(2-amino- | 192 | 508 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LVEGSPSSGAPPPSK(PEG₂₄-BEA-MalDap₂-Fc)-NH₂ | | | |
| 497 | | 1114 | Y-Aib-QGT-αMeF(2F)-TSDY-αMeS-I-αMeL-LD-Orn-K(2-12-(2-amino- | 191 | 509 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LVEGSPSSGAPPPSK(PEG₂₄-BEA-MalDap₂-Fc)-NH₂ | | | |
| 498 | | 1115 | Y-Aib-QGT-αMeF(2F)-TSDY-αMeS-I-αMeL-LD-Orn-K(2-12-(2-amino- | 98 | 468 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSGK(PEG₂₄-BEA-MalDap₂-Fc)-NH₂ | | | |
| 499 | | 1116 | Y-Aib-QGT-αMeF(2F)-TSDY-αMeS-I-αMeL-LD-Orn-K(2-12-(2-amino- | 128 | 438 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSGK(PEG₂₄-BEA-MalDap₂-Fc)-NH₂ | | | |
| 500 | | 1117 | Y-Aib-QGT-αMeF(2F)-TSD-4Pal-SI-αMeL-LD-Orn-K(ϵKϵK-(γGlu)-CO- | 94 | 510 | 935 |
| | | | (CH₂)₁₆-CO₂H)AQ-αMe4Pal-Orn-FIE-αMeY-LIEGSPSSGAPPPSK(PEG₂₄- | | | |
| | | | BEA-MalDap₂-Fc)-NH₂ | | | |
| 501 | | 1118 | Y-Aib-QGT-αMeF(2F)-TSDY-αMeS-I-αMeL-LD-Orn-K(ϵKϵK-(γGlu)-CO- | 98 | 511 | 935 |
| | | | (CH₂)₁₆-CO₂H)AQ-αMe4Pal-Orn-FIE-αMeY-LIEGSPSSGAPPPSK(PEG₂₄- | | | |
| | | | BEA-MalDap₂-Fc)-NH₂ | | | |
| 502 | | 1119 | Y-Aib-QGT-αMeF(2F)-TSD-4Pal-αMeS-I-αMeL-LD-Orn-K(ϵKϵK-(γGlu)- | 130 | 512 | 935 |
| | | | CO-(CH₂)₁₆-CO₂H)AQ-αMe4Pal-EFIE-αMeY-LIEGSPSSGAPPPSK(PEG₂₄- | | | |
| | | | BEA-MalDap₂-Fc)-NH₂ | | | |
| 503 | | 1120 | Y-Aib-QGT-αMeF(2F)-TSDY-αMeS-I-αMeL-LD-Orn-K(&K&K-(γGlu)-CO- | 128 | 513 | 935 |
| | | | (CH₂)₁₆-CO₂H)AQ-αMe4Pal-EFIE-αMeY-LIEGSPSSGAPPPSK(PEG₂₄- | | | |
| | | | BEA-MalDap₂-Fc)-NH₂ | | | |
| 504 | | 1121 | Y-Aib-QGT-αMeF(2F)-TSDY-αMeS-I-αMeL-LD-Orn-K(2-[2-(2-amino- | 193 | 514 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LVEGSPSSGAPPPSK(PEG₂₄-BEA-MalDap₂-Fc)-NH₂ | | | |
| 505 | | 1122 | YaQGT-aMcF(2F)-TSD-4Pal-αMeS-I-αMeL-LD-Orn-K(2-[2-(2-amino- | 194 | 515 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 506 | | 1123 | YaQGT-aMcF(2F)-TSD-4Pal-SI-αMeL-LD-Orn-K(2-[2-(2-amino-ethoxy)- | 195 | 516 | 935 |
| | | | ethoxy]-acetyl)-(γGlu)-CO-(CH₂)₁₆-CO₂H)AQ-αMe4Pal-Orn-FIE-αMeY- | | | |
| | | | LIEGSPSSGAPPPSK(PEG₂₄-BEA-MalDap₂-Fc)-NH₂ | | | |
| 507 | | 1124 | YaQGT-αMeF(2F)-TSDY-αMeS-I-αMeL-LD-Orn-K(2-[2-(2-amino-ethoxy)- | 196 | 517 | 935 |
| | | | ethoxy]-acetyl)-(γGlu)-CO-(CH₂)₁₆-CO₂H)AQ-αMe4Pal-Orn-FIE-αMeY- | | | |
| | | | LIEQSPSSGAPPPSK(PEG₂₄-BEA-MalDap₂-Fc)-NH₂ | | | |
| 508 | | 1125 | Y-αMeS-QGT-αMeF(2F)-TSDY-αMeS-I-αMeL-LD-Orn-K(2-[2-(2-amino- | 197 | 518 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 509 | | 1126 | Y-αMeS-QGT-αMeF(2F)-TSD-4Pal-αMeS-I-αMeL-LD-Orn-K(2-[2-(2- | 198 | 519 | 935 |
| | | | amino-ethoxy)-ethoxy]-acetyl)-(γGlu)-CO-(CH₂)₆-CO₂H)AQ-αMe4Pal-Orn- | | | |
| | | | FIE-αMeY-LIEGSPSSGAPPPSK(PEG₂₄-BEA-MalDap₂-Fc)-NH₂ | | | |
| 510 | | 1127 | Y-αMeS-QGT-αMeF(2F)-TSDY-αMeS-I-αMeL-LD-Orn-K(2-[2-(2-amino- | 197 | 520 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 511 | | 1128 | Y-Iva-QGT-αMeF(2F)-TSDY-αMeS-I-αMeL-LD-Orn-K(2-[2-(2-amino- | 199 | 521 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 512 | | 1129 | Y-Iva-QGT-αMeF(2F)-TSD-4Pal-αMeS-I-αMeL-LD-Orn-K(2-[2-(2-amino- | 200 | 522 | 935 |
| | | | ethoxy)-ethoxy]-acetyl)-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 513 | | 1130 | Y-Iva-QGT-αMeF(2F)-TSDY-αMeS-I-αMeL-LD-Orn-K(2-[2-(2-amino- | 199 | 523 | 935 |
| | | | ethoxy)-ethoxy]-acetyl)₂-(γGlu)-CO-(CH₂)₁₆-CO₂H)AQ-αMe4Pal-Orn-FIE- | | | |
| | | | αMeY-LIEGSPSSGAPPPSK(PEG₂₄-BEA-MalDap₂-Fc)-NH₂ | | | |
| 514 | | 1131 | Y-Ac4c-QGT-αMeF(2F)-TSD-4Pal-αMeS-I-αMeL-LD-Orn-K(2-[2-(2-amino- | 201 | 524 | 935 |
| | | | ethoxy)-ethoxy]-acetyl)-(γGlu)-CO-(CH2)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 515 | | 1132 | Y-Ac4c-QGT-αMeF(2F)-TSDY-αMeS-I-αMeL-LD-Orn-K(2-[2-(2-amino- | 152 | 525 | 935 |
| | | | ethoxy)-ethoxy]-acetyl)₂-(γGlu)-CO-(CH2)₁₆-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 516 | | 1133 | Y-Aib-QGT-αMeF(2F)-TSDY-αMeS-I-αMeL-LD-Orn-K(2-[2-(2-amino- | 128 | 395 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSGK(PEG₂₄-BEA-MalDap₂-Fc)-NH₂ | | | |
| 517 | | 1134 | Y-Aib-QGT-αMeF(2F)-TSDY-αMeS-I-αMeL-LD-Orn-K(ϵKϵK-(γGlu)-CO- | 113 | 526 | 935 |
| | | | (CH₂)₁₆-CO₂H)AQ-αMe4Pal-EFIe-αMeY-LIEGSPSSGAPPPSK(PEG₂₄-BEA- | | | |
| | | | MalDap₂-Fc)-NH₂ | | | |
| 518 | | 1135 | Y-Aib-QGT-αMeF(2F)-TSDY-αMeS-I-αMeL-LD-Orn-K(2-12-(2-amino- | 202 | 527 | 935 |
| | | | ethoxy)-ethoxy]-acetyl)-(γGlu)-CO-(CH₂)₁₆-CO₂H)AQ-αMeL-Orn-FIe- | | | |
| | | | αMeY-LIEGSPSSGAPPPS-K(PEG₂₄-BEA-MalDap₂-Fc)-NH₂ | | | |
| 519 | | 1136 | Y-Aib-QGT-αMeF(2F)-TSDY-αMeS-I-αMeL-LD-Orn-K(2-[2-(2-amino- | 203 | 528 | 935 |
| | | | ethoxy)-ethoxy]-acetyl)-(γGlu)-CO-(CH₂)₁₆-CO₂H)AQ-αMeL-Iva-FIe-αMeY- | | | |
| | | | LIEGSPSSGAPPPS-K(PEG₂₄-BEA-MalDap₂-Fc)-NH₂ | | | |
| 520 | | 1137 | Y-Aib-QGT-αMeF(2F)-TSD-4Pal-αMeS-I-αMeL-LD-Orn-K(2-12-(2-amino- | 204 | 529 | 935 |
| | | | ethoxy)-ethoxy]-acetyl)-(γGlu)-CO-(CH₂)₁₆-CO₂H)AQ-αMeL-Orn-FIe- | | | |
| | | | αMeY-LIEGSPSSGAPPPS-K(PEG₂₄-BEA-MalDap₂-Fc)-NH₂ | | | |
| 521 | | 1138 | Y-Aib-QGT-αMeF(2F)-TSD-4Pal-αMeS-I-αMeL-LD-Orn-K(2-12-(2-amino- | 205 | 530 | 935 |
| | | | ethoxy)-ethoxy]-acetyl)-(γGlu)-CO-(CH₂)₁₆-CO₂H)AQ-αMeL-Iva-FIe-αMeY- | | | |
| | | | LIEGSPSSGAPPPS-K(PEG₂₄-BEA-MalDap₂-Fc)-NH2 | | | |
| 522 | | 1139 | Y-Aib-QGT-αMeF(2F)-TSDY-αMeS-I-αMeL-LD-Orn-K(2-12-(2-amino- | 206 | 531 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIAGSPSSGAPPPSK(PEG₂₄-BEA-MalDap₂-Fc)-NH₂ | | | |
| 523 | | 1140 | Y-Aib-QGT-αMeF(2F)-TSDY-αMeS-I-αMeL-LD-Orn-K(2-12-(2-amino- | 206 | 532 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIAGSPSSGAPPPSK(PEG₂₄-BEA-MalDap₂-Fc)-NH₂ | | | |
| 524 | | 1141 | Y-Aib-QGT-αMeF(2F)-TSDY-αMeS-I-αMeL-LD-Orn-K(2-[2-(2-amino- | 207 | 533 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEaSPSSGAPPPSK(PEG₂₄-BEA-MalDap₂-Fc)-NH₂ | | | |
| 525 | | 1142 | Y-Aib-QGT-αMeF(2F)-TSDY-αMeS-I-αMeL-LD-Orn-K(2-12-(2-amino- | 208 | 534 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIAGSPSSGAPPPSK(PEG₂₄-BEA-MalDap₂-Fc)-NH₂ | | | |
| 526 | | 1143 | YaQGT-αMeF(2F)-TSDY-αMeS-I-αMeL-LD-Orn-K(2-[2-(2-amino-ethoxy)- | 209 | 535 | 935 |
| | | | ethoxy]-acetyl)2-(γGlu)-CO-(CH₂)₁₆-CO₂H)AQ-αMe4Pal-Orn-FIE-αMeY- | | | |
| | | | LIAGSPSSGAPPPSK(PEG₂₄-BEA-MalDap₂-Fc)-NH₂ | | | |
| 527 | | 1144 | Y-Aib-QGT-αMeF(2F)-TSDY-αMeS-I-αMeL-LD-Orn-K(2-[2-(2-amino- | 210 | 536 | 935 |
| | | | ethoxy)-ethoxy]-acetyl)-(γGlu)-CO-(CH₂)₁₆-CO₂H)AQ-Aib-Orn-FIe-αMeY- | | | |
| | | | LIAGSPSSGAPPPSK(PEG₂₄-BEA-MalDap₂-Fc)-NH₂ | | | |
| 528 | | 1145 | YaQGT-αMeF(2F)-TSDY-αMeS-I-αMeL-LD-Orn-K(2-[2-(2-amino-ethoxy)- | 211 | 537 | 935 |
| | | | ethoxy]-acetyl)₂-(γGlu)-CO-(CH₂)₁₆-CO₂H)AQ-αMe4Pal-Orn-FIE-αMeY- | | | |
| | | | LIEaSPSSGAPPPSK(PEG₂₄-BEA-MalDap₂-Fc)-NH₂ | | | |
| 529 | | 1146 | Y-Aib-QGT-αMeF(2F)-TSDY-αMeS-I-αMeL-LD-Orn-K(2-12-(2-amino- | 212 | 538 | 935 |
| | | | ethoxy)-ethoxy]-acetyl)₂-(γGlu)-CO-(CH2)₁₆-CO₂H)AQ-αMe4Pal-SFIE- | | | |
| | | | αMeY-LIEGSPSSGAPPPSK(PEG₂₄-BEA-MalDap₂-Fc)-NH₂ | | | |
| 530 | | 1147 | Y-Aib-QGT-αMeF(2F)-TSDY-αMeS-I-αMeL-LD-Orn-K(2-12-(2-amino- | 213 | 539 | 935 |
| | | | ethoxy)-ethoxy]-acetyl)₂-(γGlu)-CO-(CH₂)₁₆-CO₂H)AQ-Aib-SFIE-αMeY- | | | |
| | | | LIEGSPSSGAPPPSK(PEG₂₄-BEA-MalDap₂-Fc)-NH₂ | | | |
| 531 | | 1148 | Y-Aib-QGT-αMeF(2F)-TSDY-αMeS-I-αMeL-LD-Orn-K(2-[2-(2-amino- | 214 | 540 | 935 |
| | | | ethoxy)-ethoxy]-acetyl)₂-(γGlu)-CO-(CH2)₁₆-CO₂H)AQ-4Pal-AFIE-αMeY- | | | |
| | | | LIEGSPSSGAPPPSK(PEG₂₄-BEA-MalDap₂-Fc)-NH₂ | | | |
| 532 | | 1149 | Y-Aib-QGT-αMeF(2F)-TSDY-αMeS-I-αMeL-LD-Orn-K(2-12-(2-amino- | 215 | 541 | 935 |
| | | | ethoxy)-ethoxy]-acetyl)-(γGlu)-CO-(CH2)₁₆-CO₂H)AQ-αMeF-Aib-FIe-αMeY- | | | |
| | | | LIEGSPSSGAPPPSK(PEG₂₄-BEA-MalDap₂-Fc)-NH₂ | | | |
| 533 | | 1150 | Y-Aib-QGT-αMeF(2F)-TSDY-αMeS-I-αMeL-LD-Orn-K(2-12-(2-amino- | 216 | 542 | 935 |
| | | | ethoxy)-ethoxy]-acetyl)-(γGlu)-CO-(CH₂)₁₆-CO₂H)AQ-Iva-AFIe-αMeY- | | | |
| | | | LIEGSPSSGAPPPS-K(PEG₂₄-BEA-MalDap₂-Fc)-NH₂ | | | |
| 534 | | 1151 | Y-Aib-QGT-αMeF(2F)-TSDY-αMeS-I-αMeL-LD-Orn-K(2-[2-(2-amino- | 217 | 543 | 935 |
| | | | ethoxy)-ethoxy]-acetyl)₂-(γGlu)-CO-(CH₂)₁₆-CO₂H)AQ-Aib-EFIE-αMeY- | | | |
| | | | LIEGSPSSGAPPPSK(PEG₂₄-BEA-MalDap₂-Fc)-NH₂ | | | |
| 535 | | 1152 | Y-Aib-QGT-αMeF(2F)-TSDY-αMeS-I-αMeL-LD-Orn-K(2-12-(2-amino- | 218 | 544 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIE-Aib-SPSSGAPPPSK(PEG₂₄-BEA-MalDap₂-Fc)-NH₂ | | | |
| 536 | | 1153 | Y-Aib-QGT-αMeF(2F)-TSDY-αMeS-I-αMeL-LD-Orn-K(2-[2-(2-amino- | 219 | 545 | 935 |
| | | | ethoxy)-ethoxy]-acetyl)-(γGlu)-CO-(CH₂)₁₆-CO₂H)AQ-αMeL-Orn-FIe- | | | |
| | | | αMeY-LIAaSPSSGAPPPS-K(PEG₂₄-BEA-MalDap₂-Fc)-NH₂ | | | |
| 537 | | 1154 | Y-Aib-QGT-αMeF(2F)-TSDY-αMeS-I-αMeL-LD-Orn-K(2-12-(2-amino- | 128 | 438 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-MSPT2-Fc)-NH₂ | | | |
| 538 | | 1155 | Y-Aib-QGT-αMeF(2F)-TSD-4Pal-αMeS-I-αMeL-LD-Orn-K(2-12-(2-amino- | 133 | 546 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIE-Aib-SPSSGAPPPSK(PEG₂₄-BEA-MSPT2-Fc)-NH₂ | | | |
| 539 | | 1156 | Y-Aib-QGT-αMeF(2F)-TSD-4Pal-αMeS-I-αMeL-LD-Orn-K(2-[2-(2-amino- | 220 | 547 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIAGSPSSGAPPPSK(PEG₂₄-BEA-MSPT2-Fc)-NH₂ | | | |
| 540 | | 1157 | Y-Aib-QGT-αMeF(2F)-TSD-4Pal-αMeS-I-αMeL-LD-Orn-K(2-[2-(2-amino- | 221 | 548 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIAaSPSSGAPPPSK(PEG₂₄-BEA-MSPT2-Fc)-NH₂ | | | |
| 541 | | 1158 | Y-Aib-QGT-αMeF(2F)-TSD-4Pal-αMeS-I-αMeL-LD-Orn-K(2-[2-(2-amino- | 222 | 549 | 935 |
| | | | ethoxy)-ethoxy]-acetyl)-(γGlu)-CO-(CH₂)₁₆-CO₂H)AQ-αMeL-Iva-FIe-αMeY- | | | |
| | | | LIA-Aib-SPSSGAPPPS-K(PEG₂₄-BEA-MSPT2-Fc)-NH₂ | | | |
| 542 | | 1159 | Y-Aib-QGT-αMeF(2F)-TSD-4Pal-αMeS-I-αMeL-LD-Orn-K(2-12-(2-amino- | 223 | 550 | 935 |
| | | | ethoxy)-ethoxy]-acetyl)₂-(γGlu)-CO-(CH2)₁₆-CO₂H)AQ-αMe4Pal-SFIE- | | | |
| | | | αMeY-LIEGSPSSGAPPPSK(PEG₂₄-BEA-MSPT2-Fc)-NH₂ | | | |
| 543 | | 1160 | Y-Aib-QGT-αMeF(2F)-TSD-4Pal-αMeS-I-αMeL-LD-Orn-K(2-[2-(2-amino- | 224 | 551 | 935 |
| | | | ethoxy)-ethoxy]-acetyl)₂-(γGlu)-CO-(CH₂)₁₆-CO₂H)AQ-αMe4Pal-SFIE- | | | |
| | | | αMeY-LIE-Aib-SPSSGAPPPSK(PEG₂₄-BEA-MSPT2-Fc)-NH₂ | | | |
| 544 | | 1161 | Y-Aib-QGT-αMeF(2F)-TSDY-αMeS-I-αMeL-LD-Orn-K(2-[2-(2-amino- | 225 | 552 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEGGGK(PEG₂₄-BEA-MSPT2-Fc)-NH₂ | | | |
| 545 | | 1162 | Y-Aib-QGT-αMeF(2F)-TSDY-αMeS-I-αMeL-LD-Orn-K(2-[2-(2-amino- | 226 | 553 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIAGGGK(PEG₂₄-BEA-MSPT2-Fc)-NH₂ | | | |
| 546 | | 1163 | Y-Aib-QGT-αMeF(2F)-TSDY-αMeS-I-αMeL-LD-Orn-K(2-12-(2-amino- | 225 | 554 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGGGK(PEG₂₄-BEA-MSPT2-Fc)-NH₂ | | | |
| 547 | | 1164 | Y-Aib-QGT-αMeF(2F)-TSDY-αMeS-I-αMeL-LD-Orn-K(2-[2-(2-amino- | 227 | 555 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIEGGGK(PEG₂₄-BEA-MSPT2-Fc)-NH₂ | | | |
| 548 | | 1165 | Y-Ac4c-QGT-αMeF(2F)-TSDY-αMeS-I-αMeL-LD-Orn-K(2-[2-(2-amino- | 228 | 556 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGGGK(PEG₂₄-BEA-MSPT2-Fc)-NH₂ | | | |
| 549 | | 1166 | Y-Aib-QGT-αMeF(2F)-TSD-4Pal-αMeS-I-αMeL-LD-Orn-K(2-[2-(2-amino- | 229 | 557 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGGGK(PEG₂₄-BEA-MSPT2-Fc)-NH₂ | | | |
| 550 | | 1167 | Y-Aib-QGT-αMeF(2F)-TSDY-αMeS-I-αMeL-LD-Orn-K(2-[2-(2-amino- | 128 | 558 | 935 |
| | | | ethoxy)-ethoxy]-acetyl)₂-(γGlu)-CO-(CH₂)₁₄-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 551 | | 1168 | Y-Aib-QGT-αMeF(2F)-TSD-4Pal-αMeS-I-αMeL-LD-Orn-K(2-[2-(2-amino- | 130 | 559 | 935 |
| | | | ethoxy)-ethoxy]-acetyl)-(γGlu)-CO-(CH2)₁₄-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 552 | | 1169 | Y-Aib-QGT-αMeF(2F)-TSDY-αMeS-I-αMeL-LD-Orn-K(2-12-(2-amino- | 128 | 560 | 935 |
| | | | ethoxy)-ethoxy]-acetyl)-(γGlu)-CO-(CH₂)₁₄-CO₂H)AQ-αMe4Pal-EFIE- | | | |
| | | | αMeY-LIEGSPSSGAPPPSK(PEG₂₄-BEA-MalDap₂-Fc)-NH₂ | | | |
| 553 | | 1170 | Y-Aib-QGT-αMeF(2F)-TSD-4Pal-αMeS-I-αMeL-LD-Orn-K(2-[2-(2-amino- | 183 | 561 | 935 |
| | | | ethoxy)-ethoxy]-acetyl)-(γGlu)-CO-(CH₂)₁₄-CO₂H)AQ-αMe4Pal-EFIe-αMeY- | | | |
| | | | LIEGSPSSGAPPPSK(PEG₂₄-BEA-MSPT2-Fc)-NH₂ | | | |
| 554 | | 1171 | Y-Aib-QGT-αMeF(2F)-TSDY-αMeS-I-αMeL-LD-Orn-K(2-[2-(2-amino- | 128 | 423 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEGSPSSGAPPPSK(PEG₂₄-BEA-Acetyl2-Fc)-NH₂ | | | |
| 555 | 40 | 1172 | Y-Aib-QGT-αMeF(2F)-TSDY-αMeS-I-αMeL-LD-Orn-K(2-12-(2-amino- | 128 | 423 | 935 |
| | | | ethoxy)-ethoxy]-acetyl)₂-(γGlu)-CO-(CH₂)₁₆-CO₂H)AQ-αMe4Pal-EFIE- | | | |
| | | | αMeY-LIEGSPSSGAPPPSK(PEG₂₄-BEA-B-MalDap₂-Fc)-NH₂ | | | |
| 556 | 41 | 1173 | Y-Aib-QGT-aMcF(2F)-TSD-4Pal-αMeS-I-αMeL-LD-Orn-K(2-[2-(2-amino- | 130 | 490 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIEGSPSSGAPPPSK(PEG₂₄-BEA-B-MalDap₂-Fc)-NH₂ | | | |
| 557 | 42 | 1174 | Y-Aib-QGT-aMcF(2F)-TSDY-αMeS-I-αMeL-LD-Orn-K(2-12-(2-amino- | 113 | 467 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIEGSPSSGAPPPSK(PEG₂₄-BEA-B-MalDap₂-Fc)-NH2 | | | |
| 558 | 43 | 1175 | Y-Aib-QGT-aMcF(2F)-TSD-4Pal-αMeS-I-αMeL-LD-Orn-K(2-[2-(2-amino- | 183 | 493 | 935 |
| | | | ethoxy)-ethoxy]-acetyl)-(γGlu)-CO-(CH₂)₁₆-CO₂H)AQ-αMe4Pal-EFIe-αMeY- | | | |
| | | | LIEGSPSSGAPPPSK(PEG₂₄-BEA-B-MalDap₂-Fc)-NH₂ | | | |
| 559 | | 1176 | Y-Aib-QGT-αMeF(2F)-TSD-4Pal-αMeS-I-αMeL-LD-Orn-K(2-[2-(2-amino- | 220 | 547 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIAGSPSSGAPPPSK(PEG₂₄-BEA-MalDap₂-Fc)-NH₂ | | | |
| 560 | | 1177 | Y-Aib-QGT-αMeF(2F)-TSD-4Pal-αMeS-I-αMeL-LD-Orn-K(2-[2-(2-amino- | 220 | 547 | 935 |
| | | | ethoxy)-ethoxy]-acetyl)-(γGlu)-CO-(CH₂)₁₆-CO₂H)AQ-αMe4Pal-EFIE- | | | |
| | | | αMeY-LIAGSPSSGAPPPSK(PEG₂₄-BEA-B-MalDap₂-Fc)-NH₂ | | | |

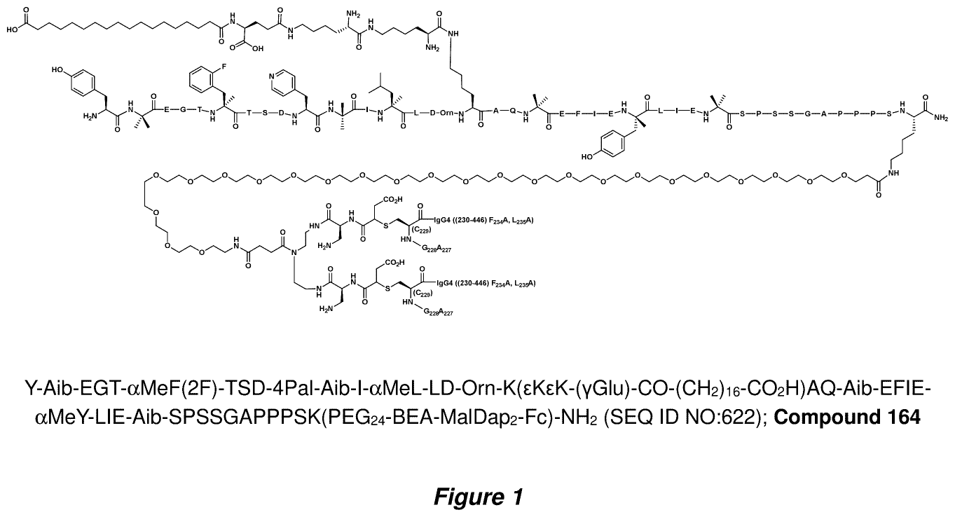

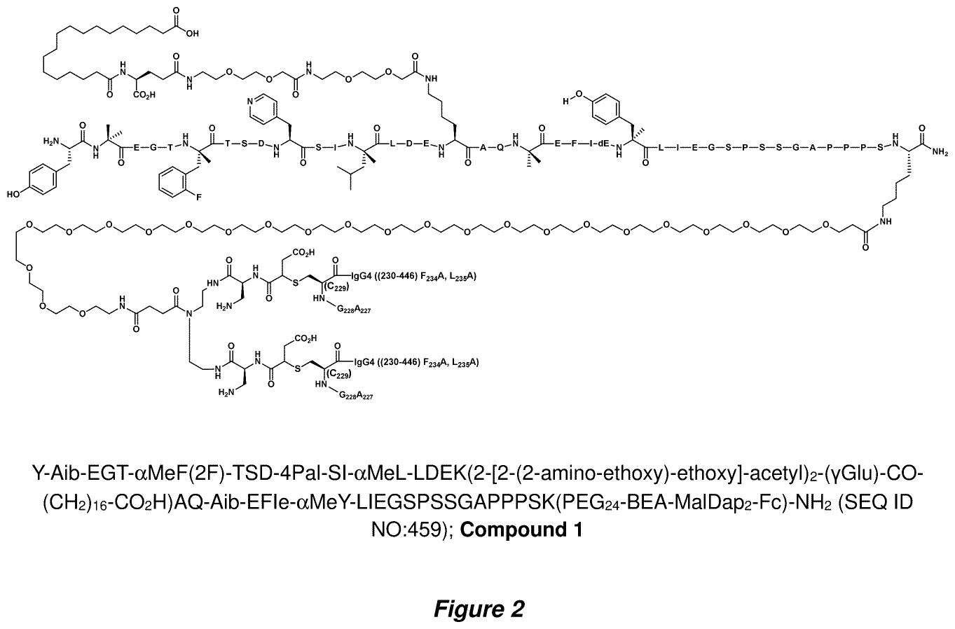

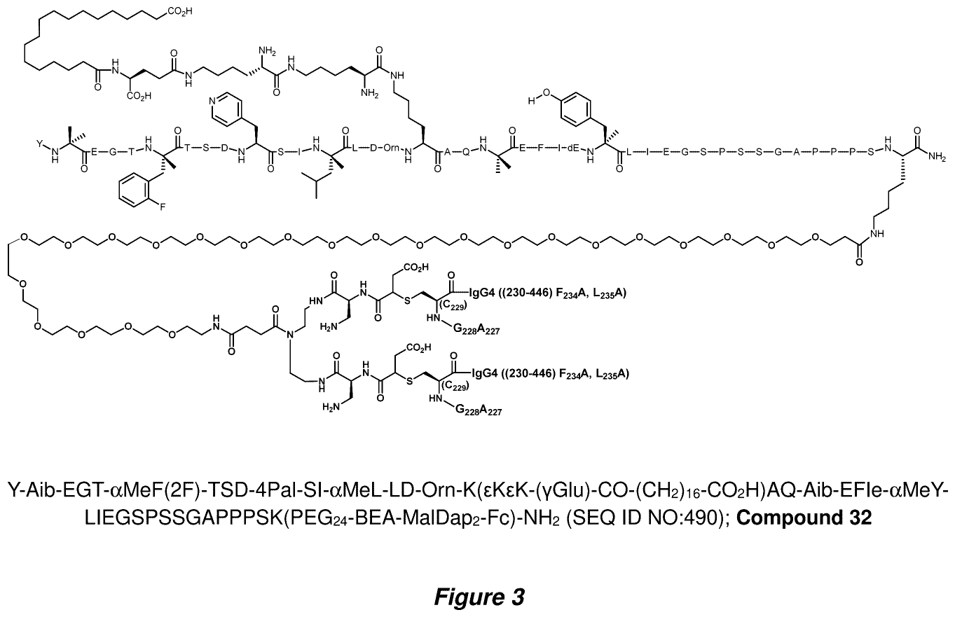

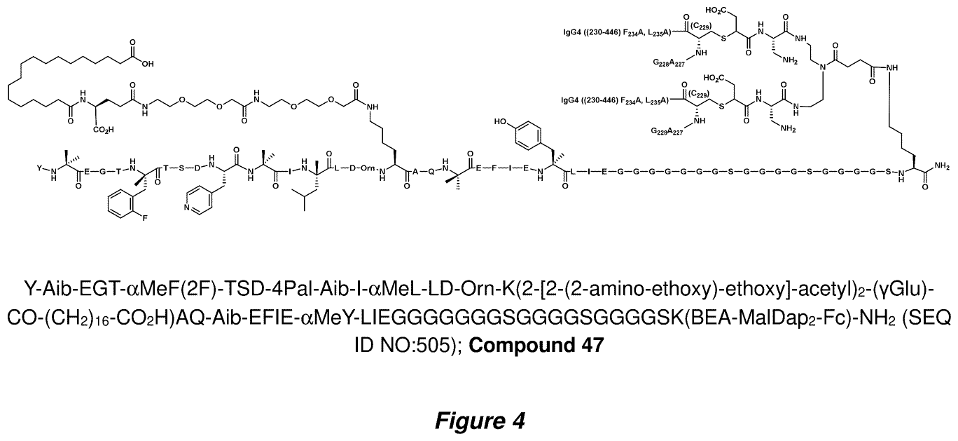

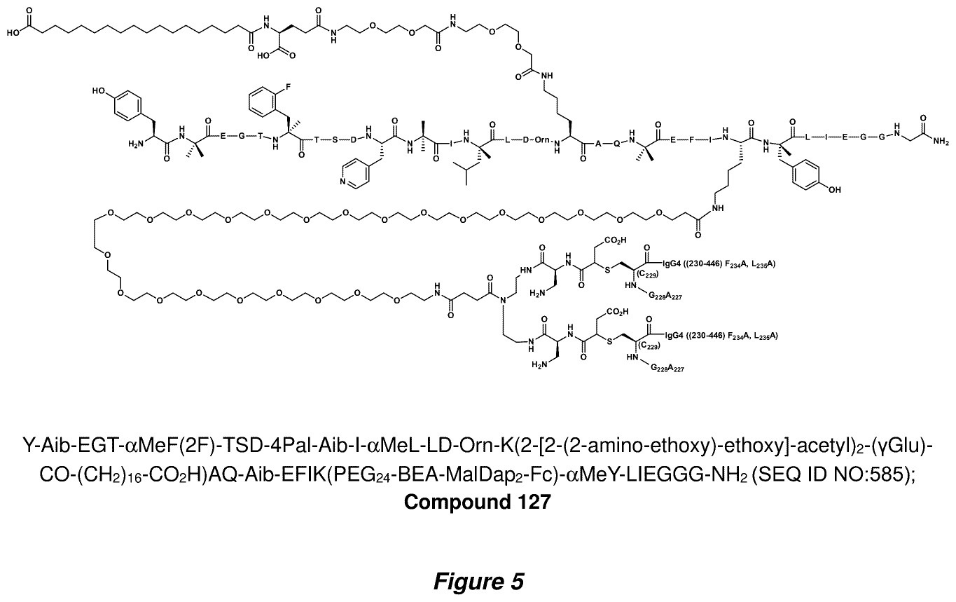

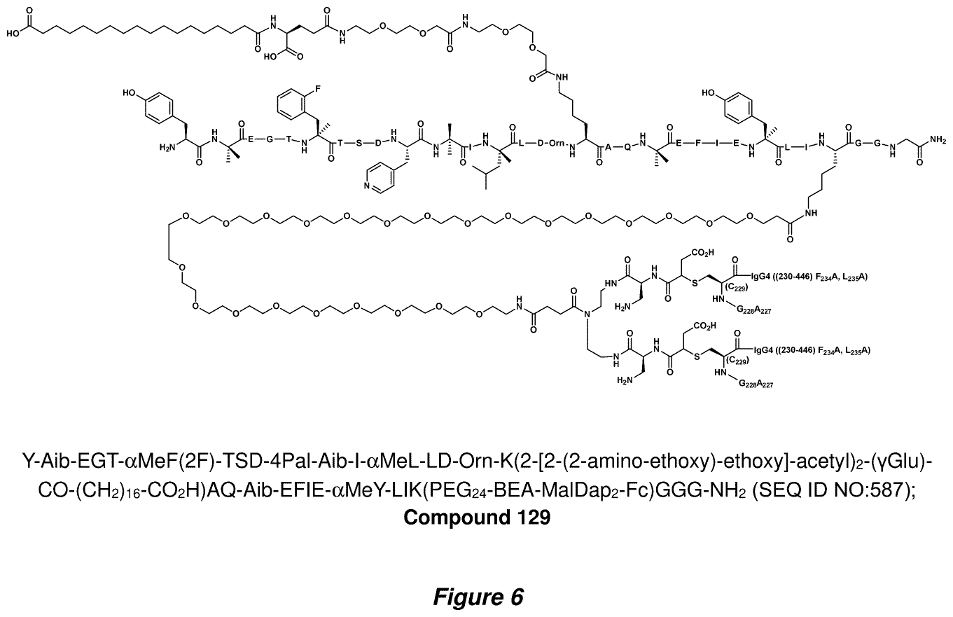

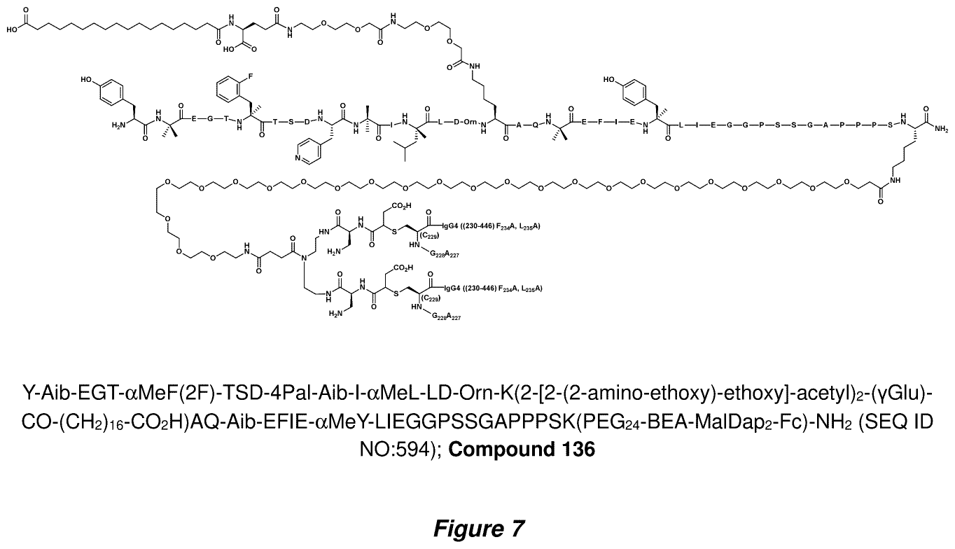

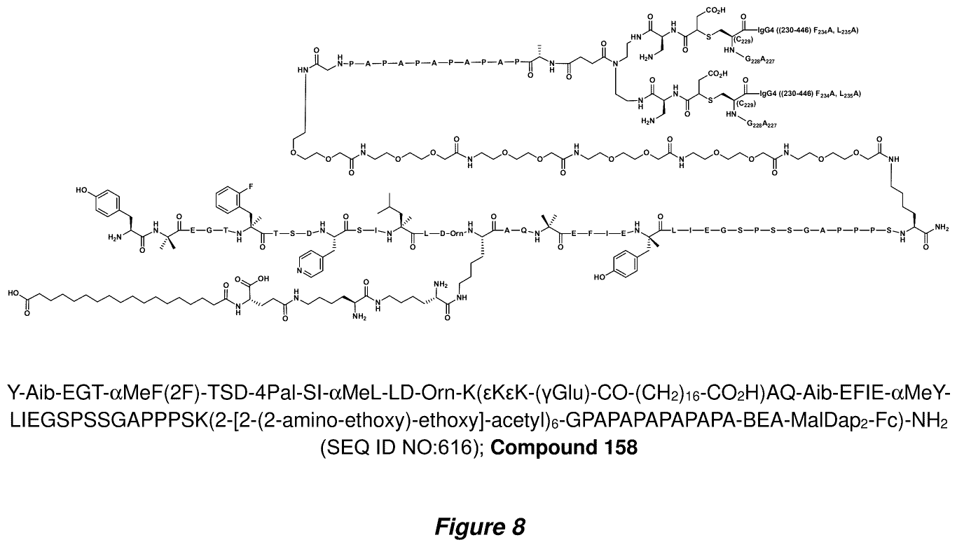

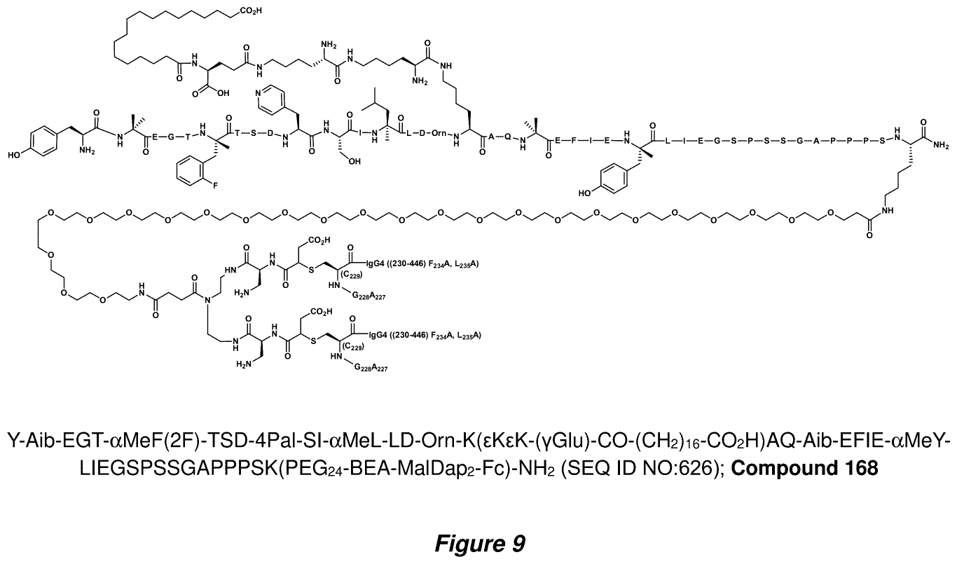

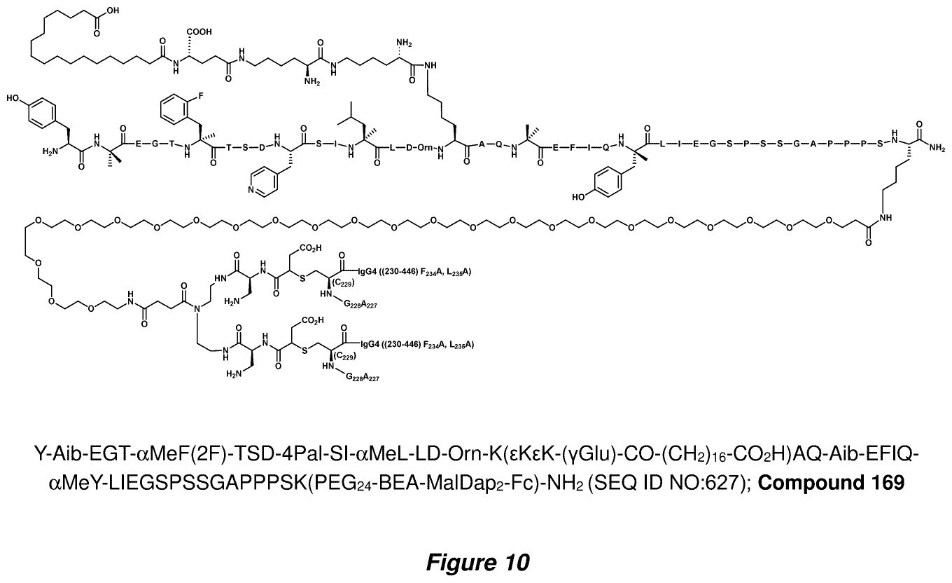

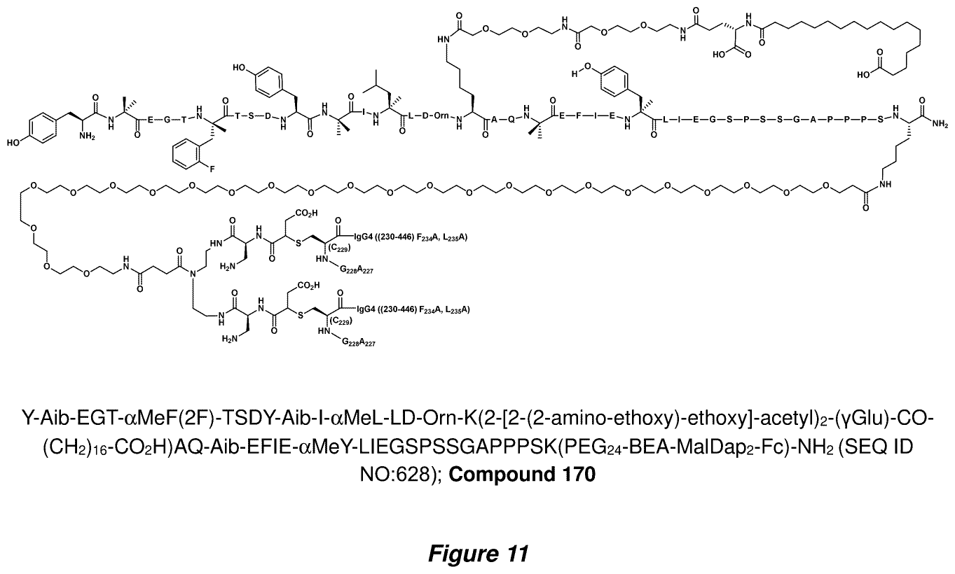

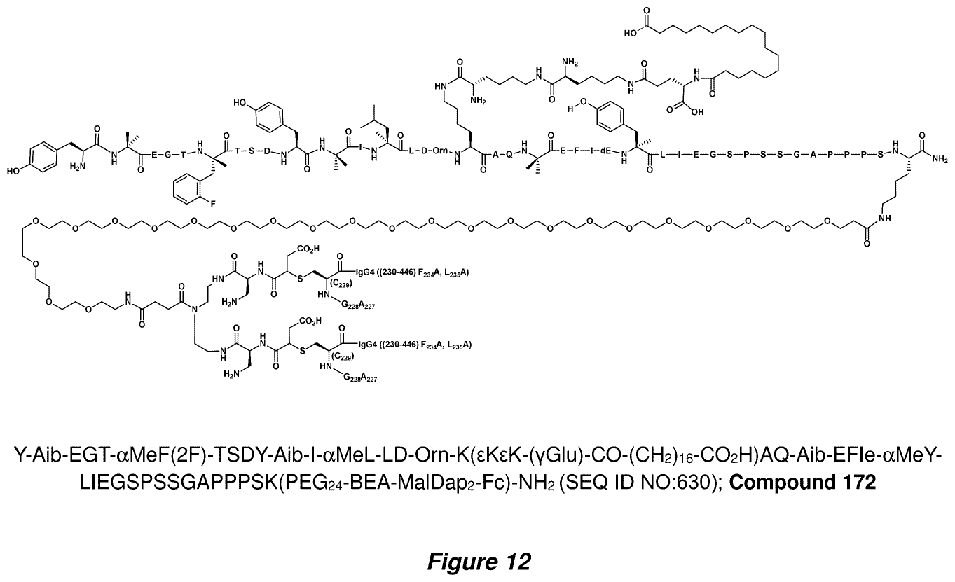

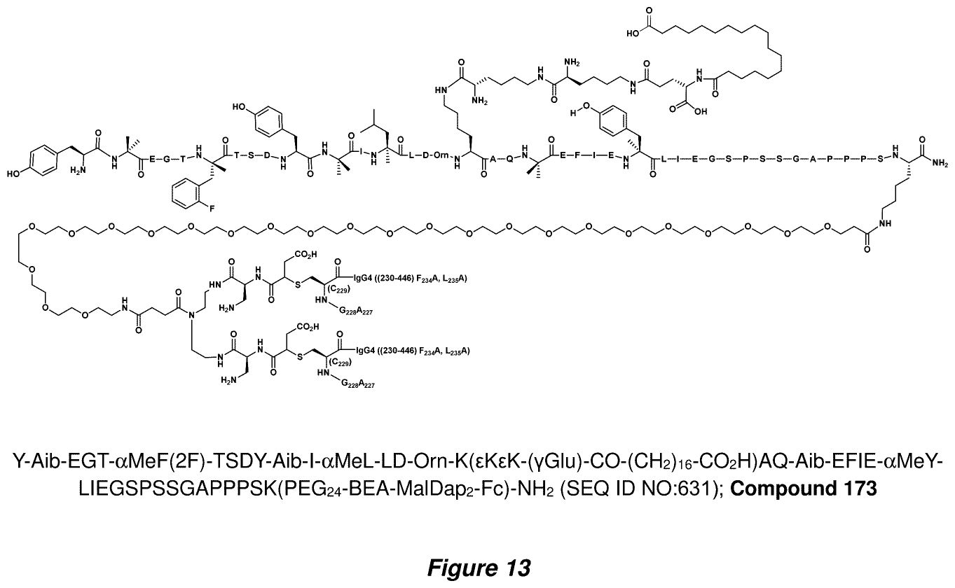

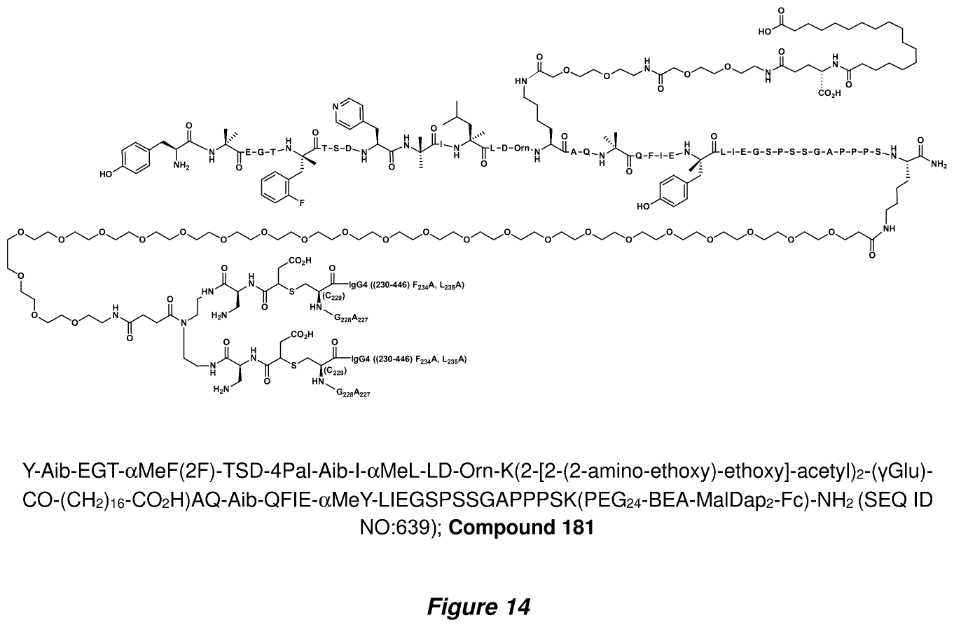

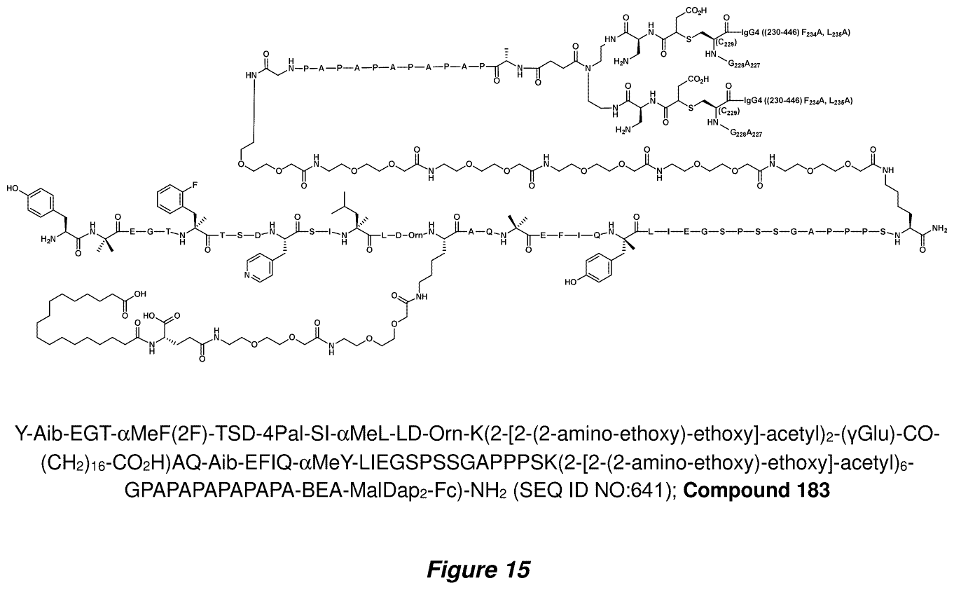

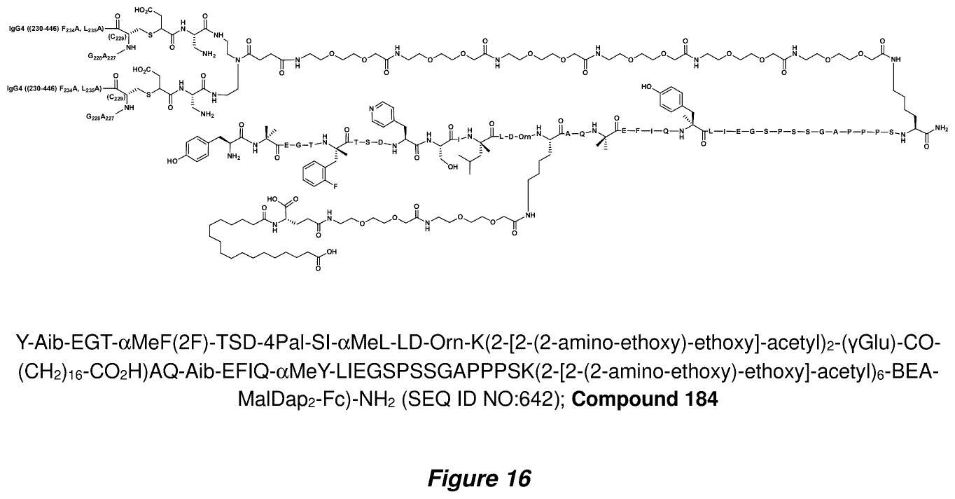

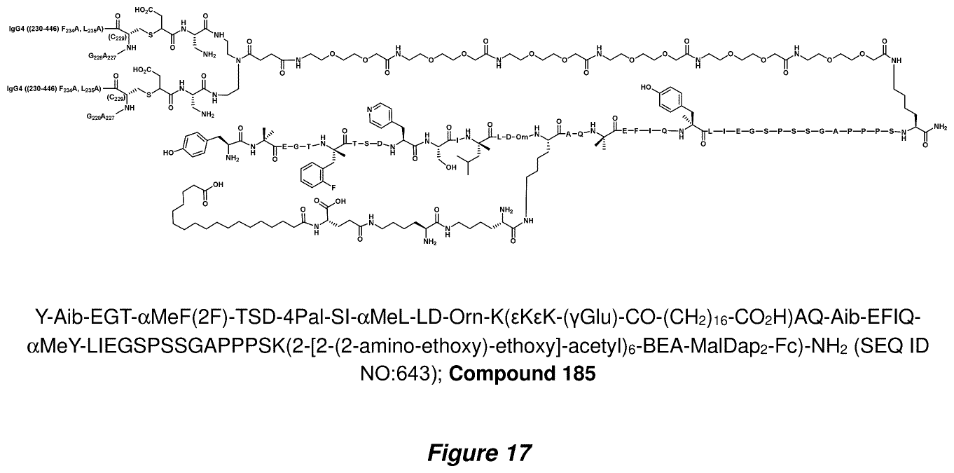

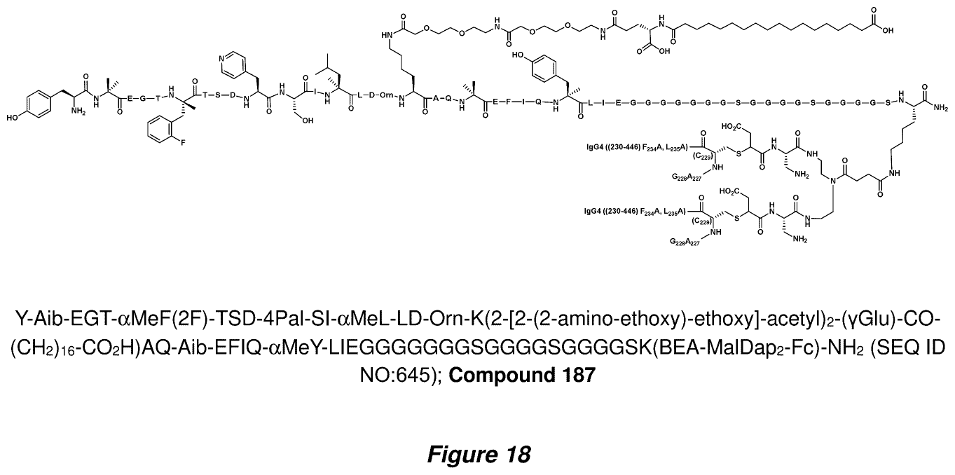

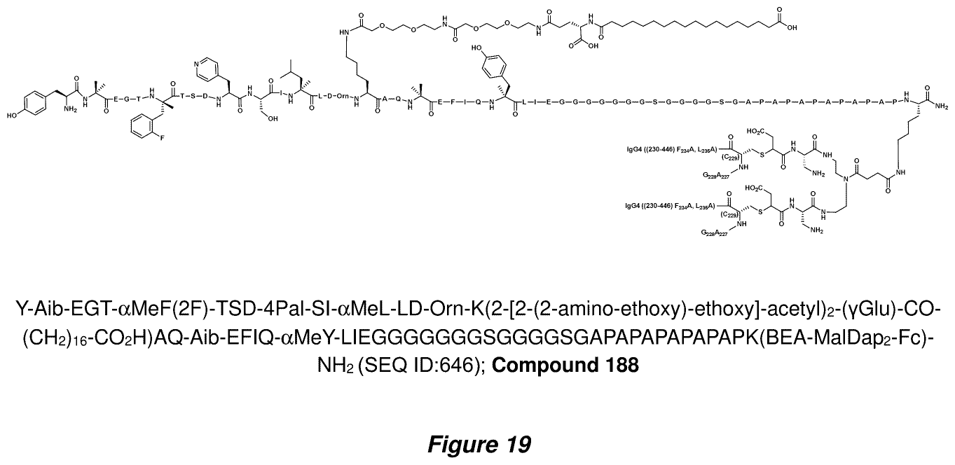

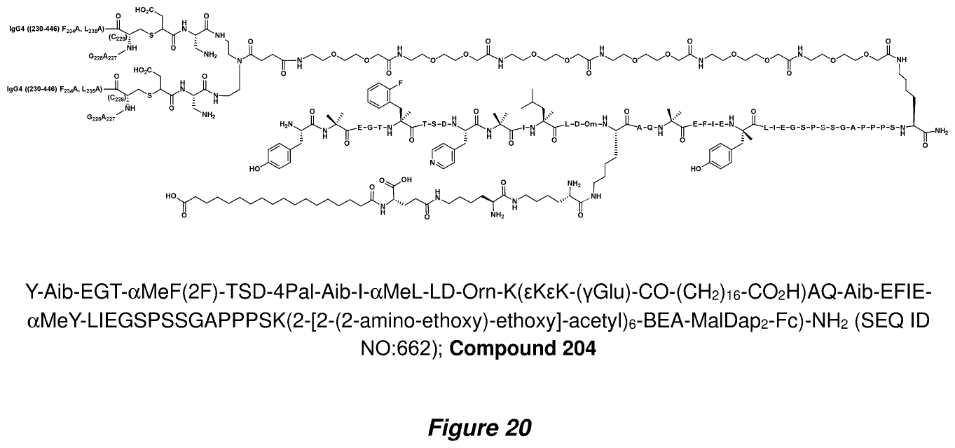

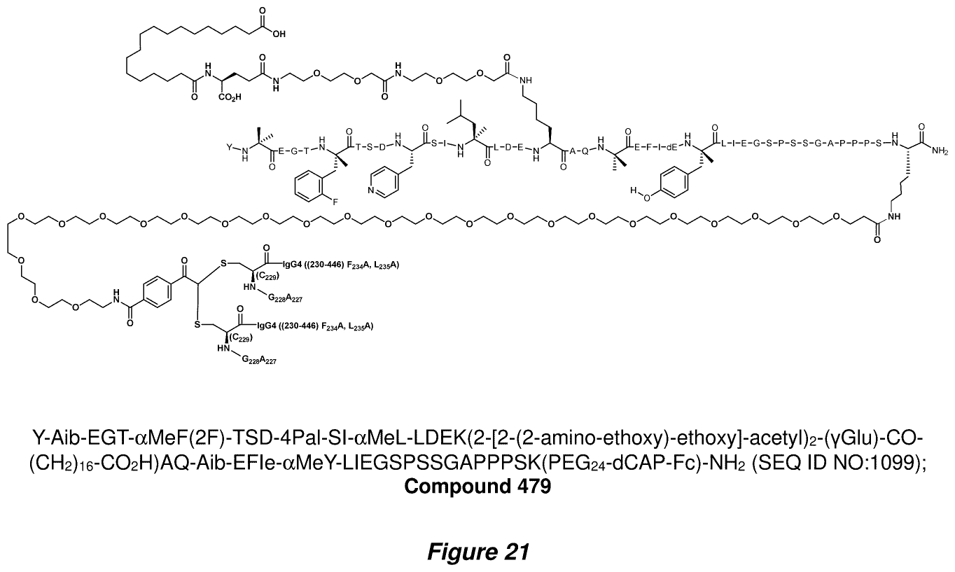

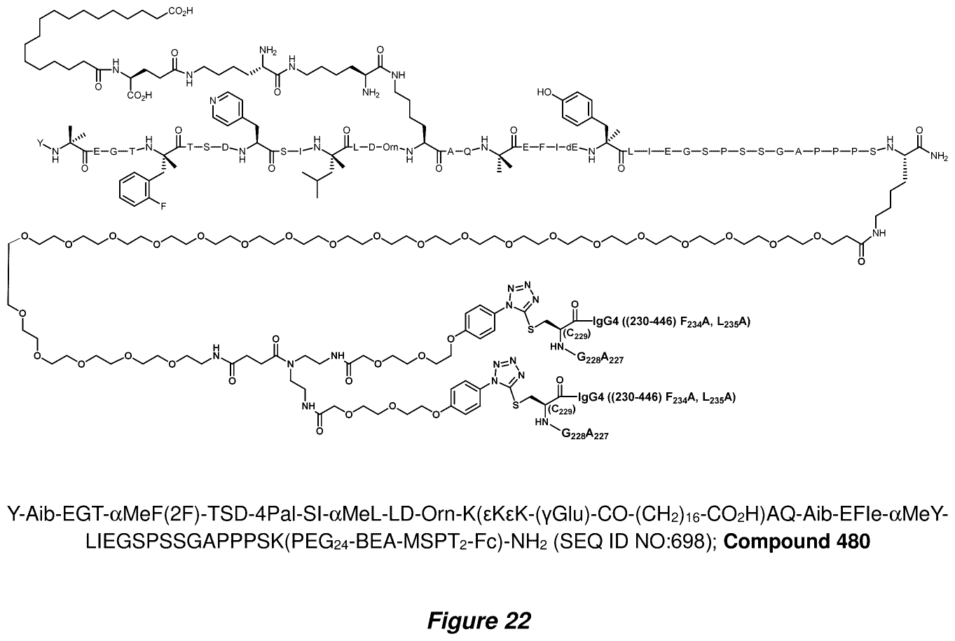

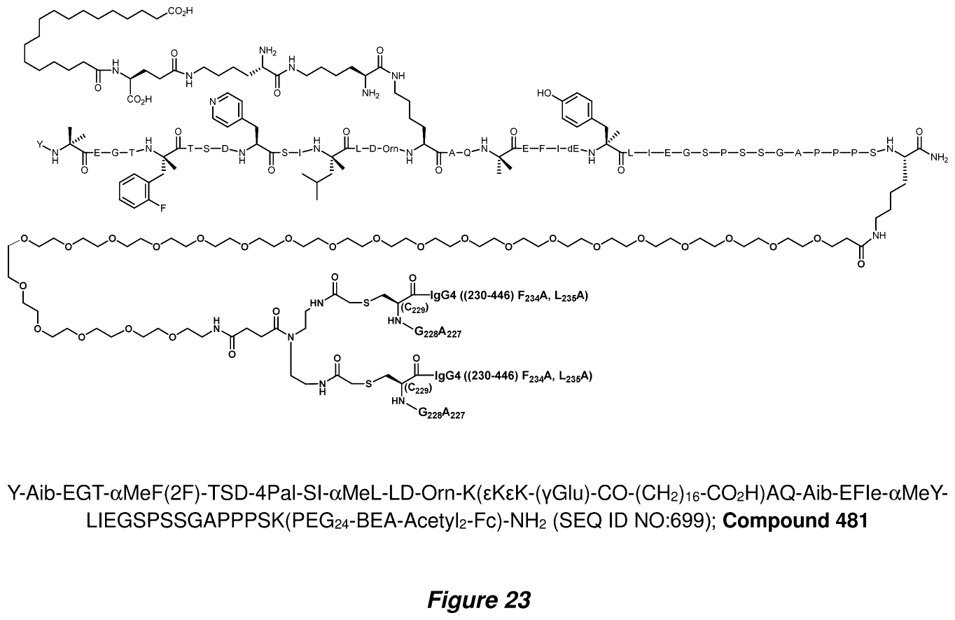

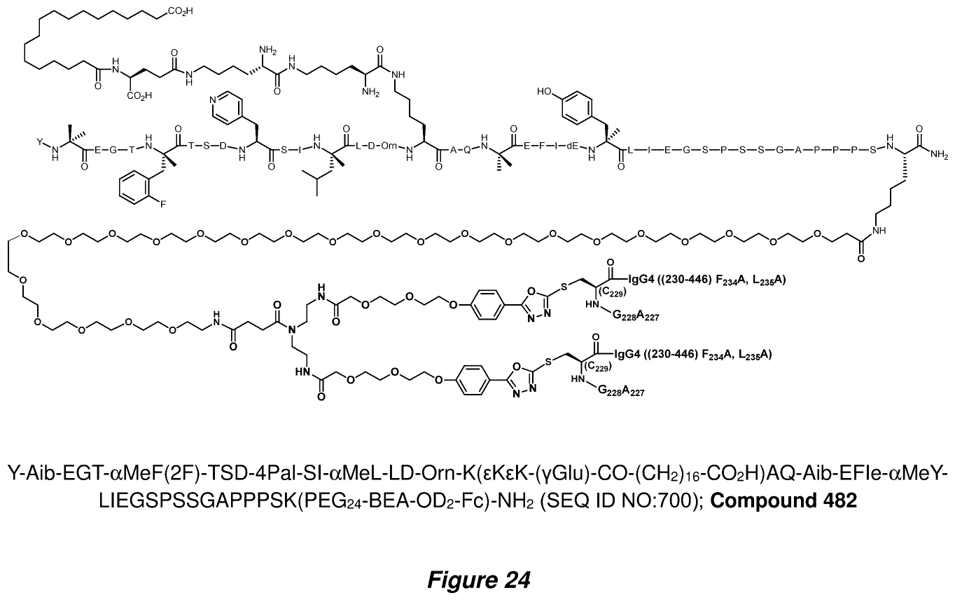

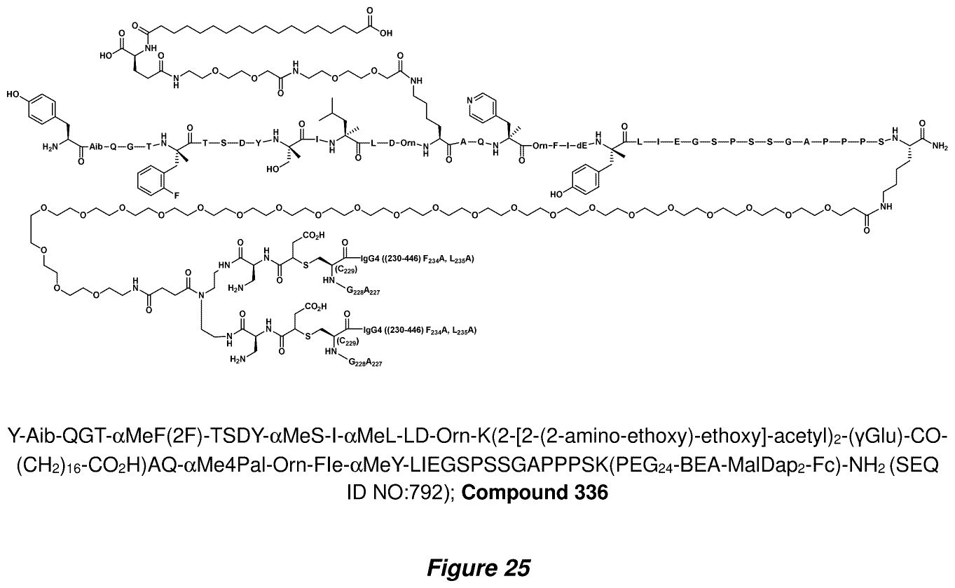

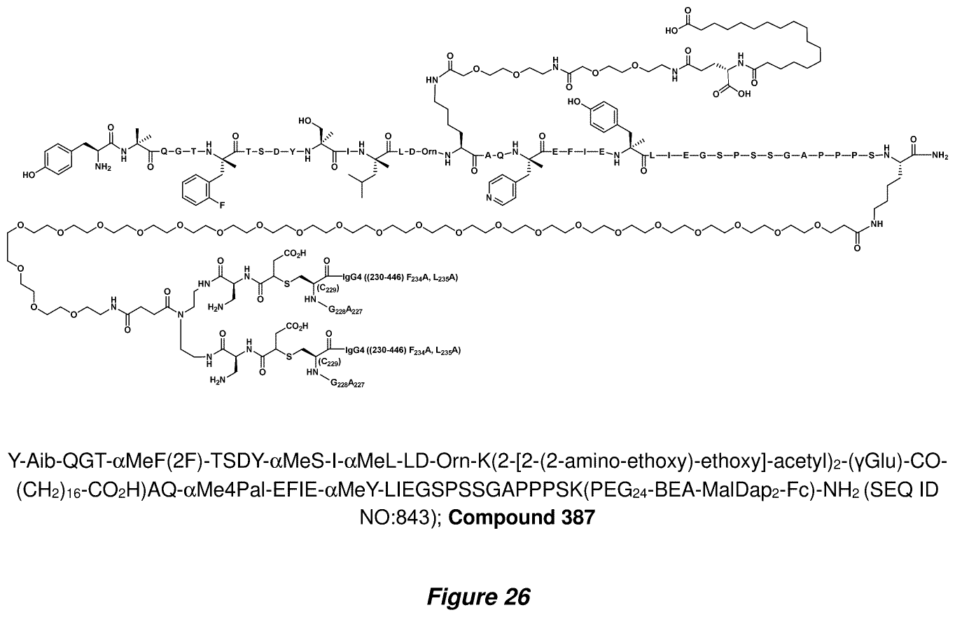

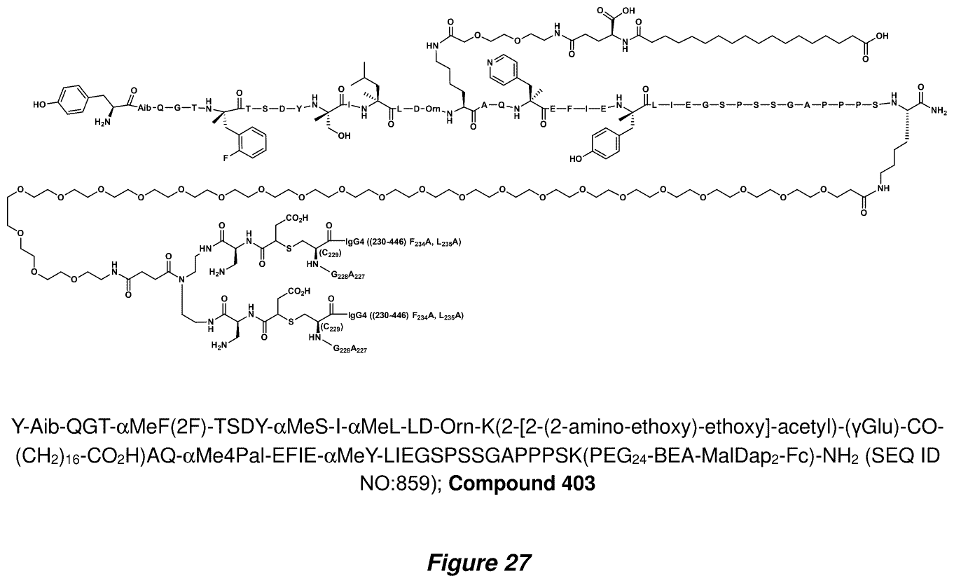

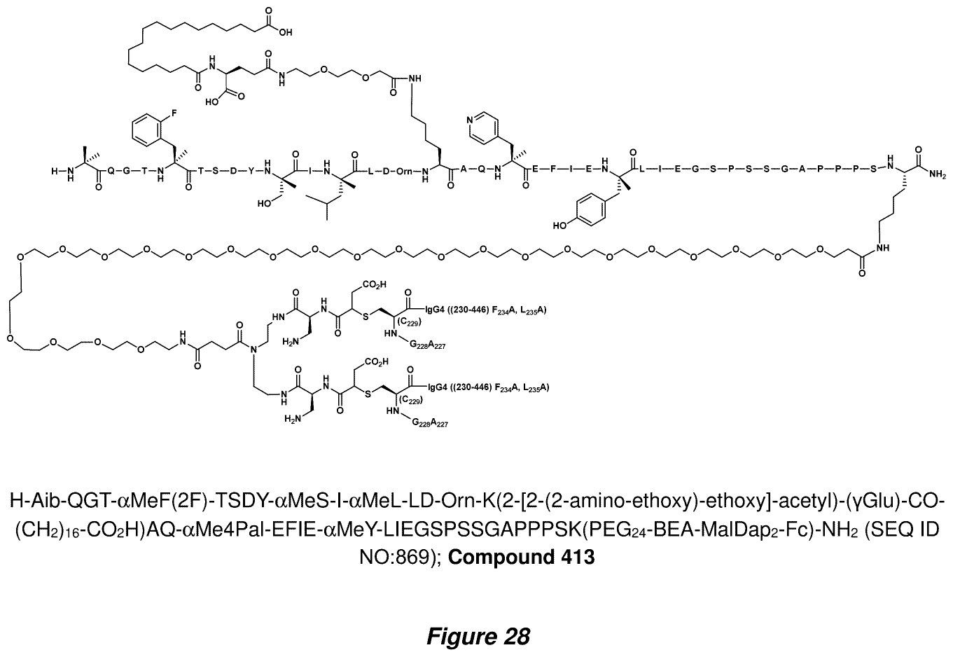

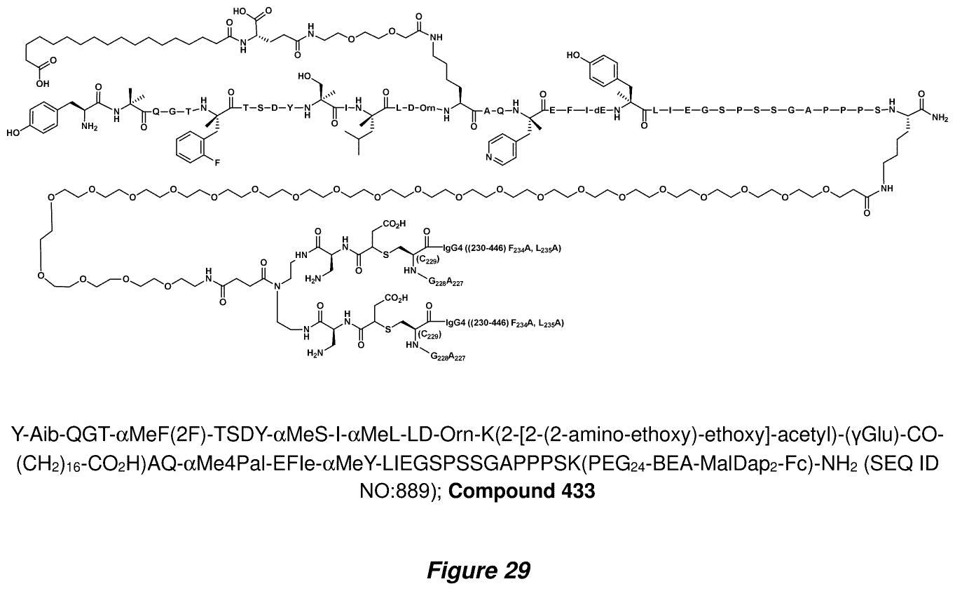

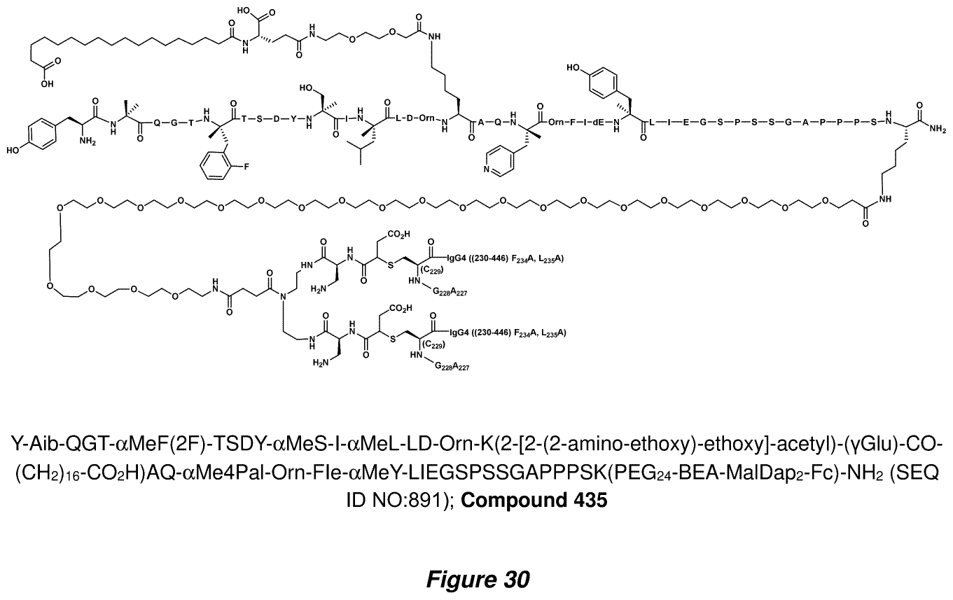

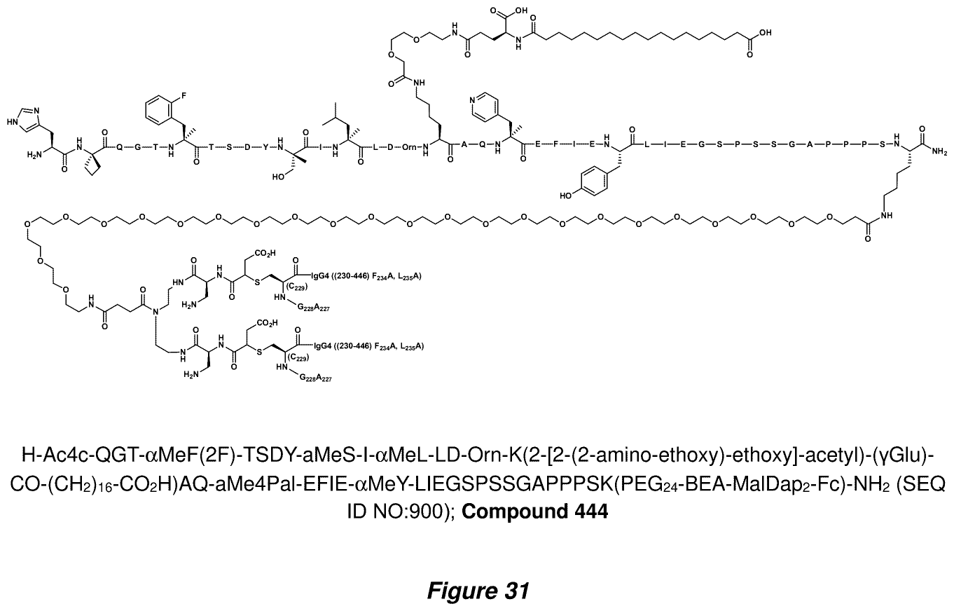

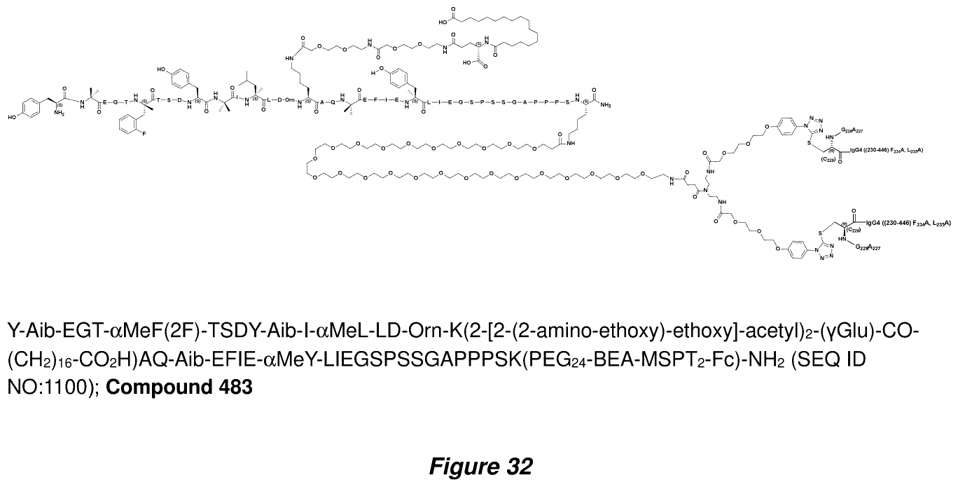

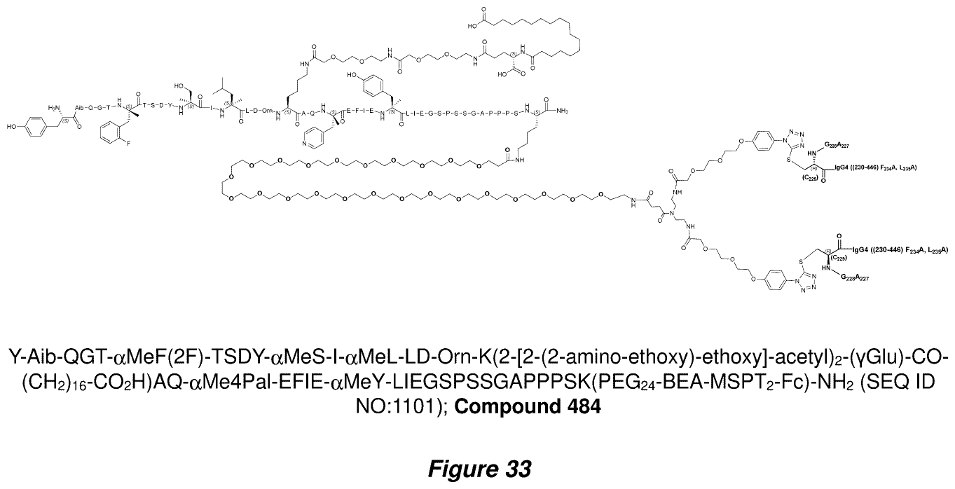

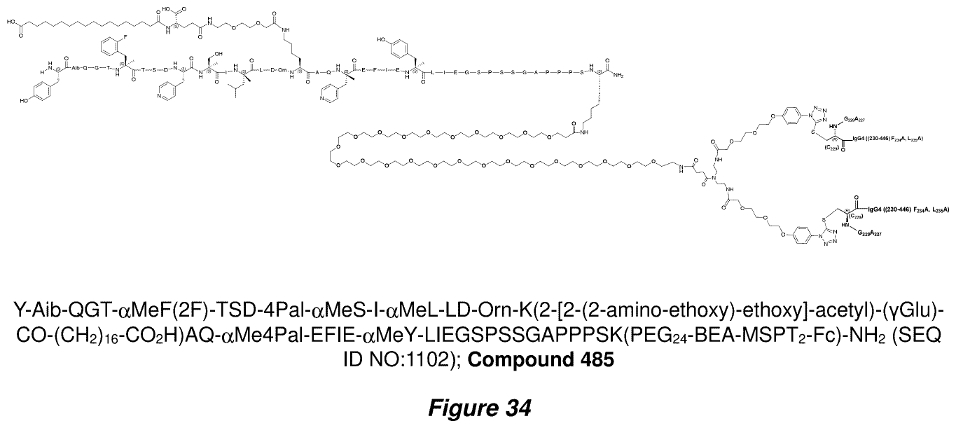

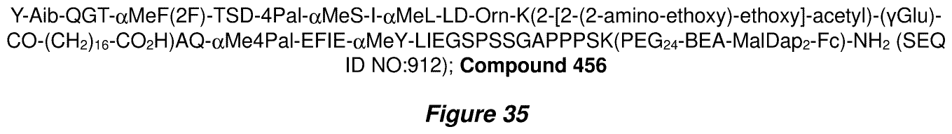

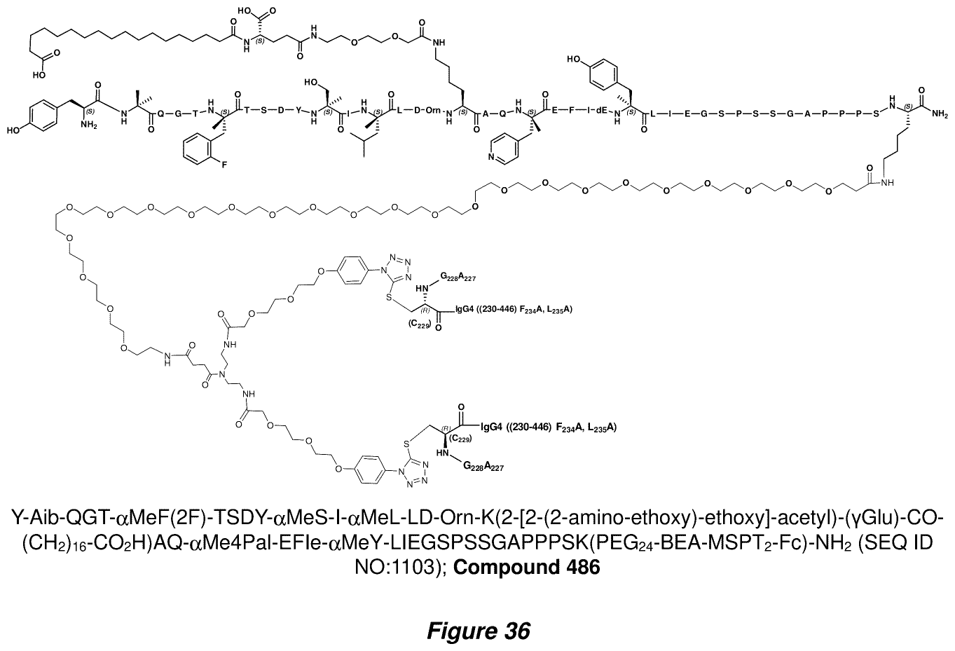

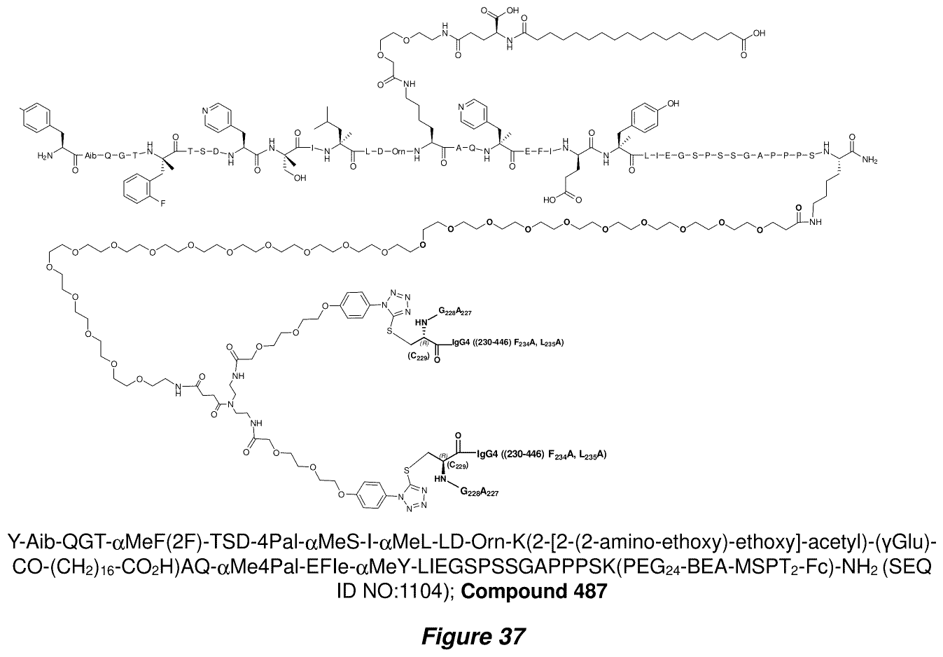

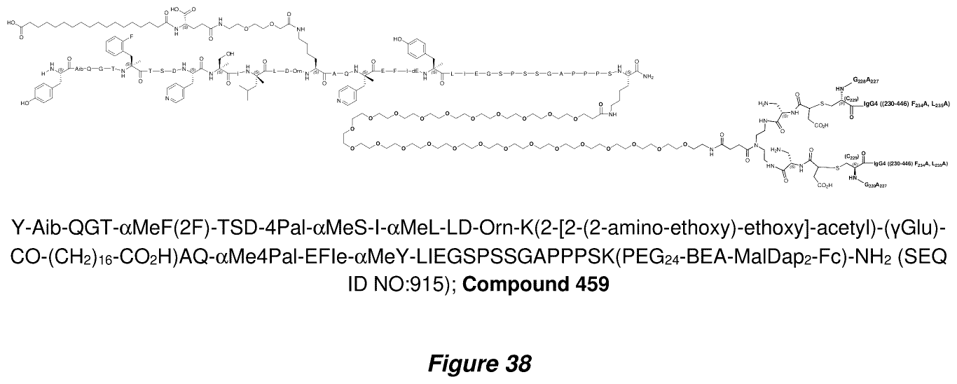

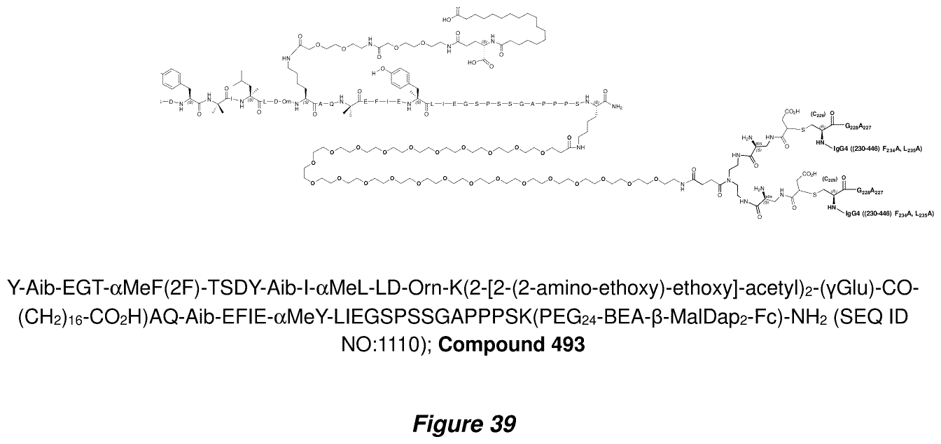

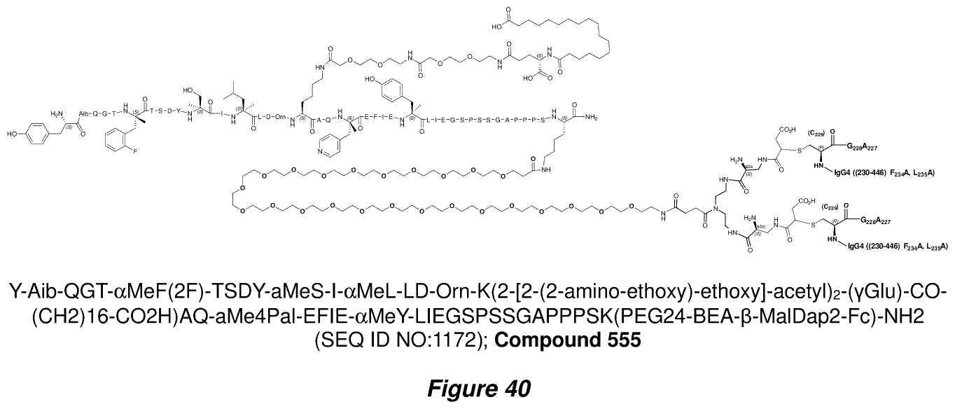

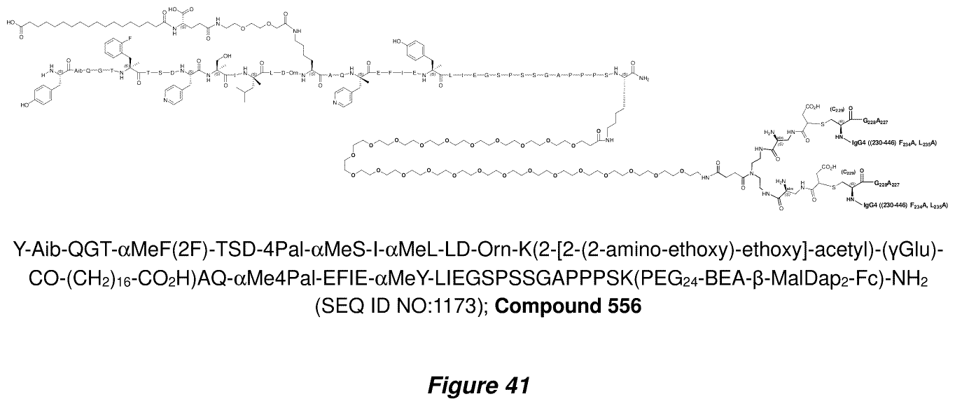

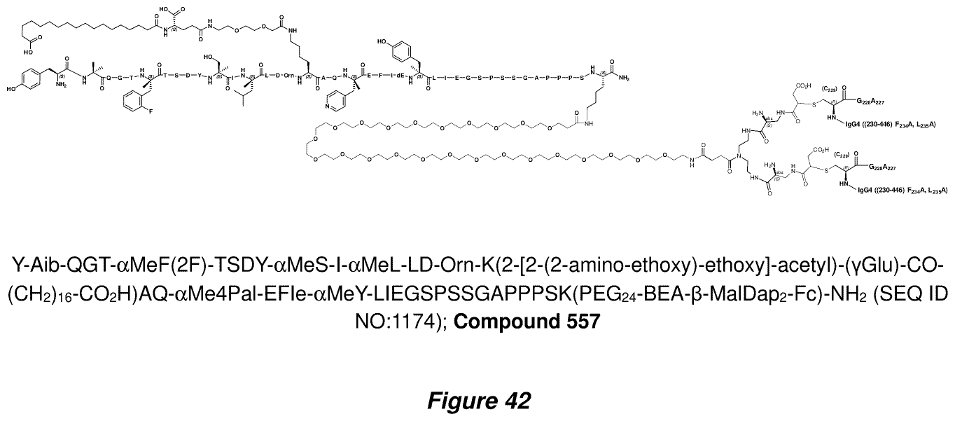

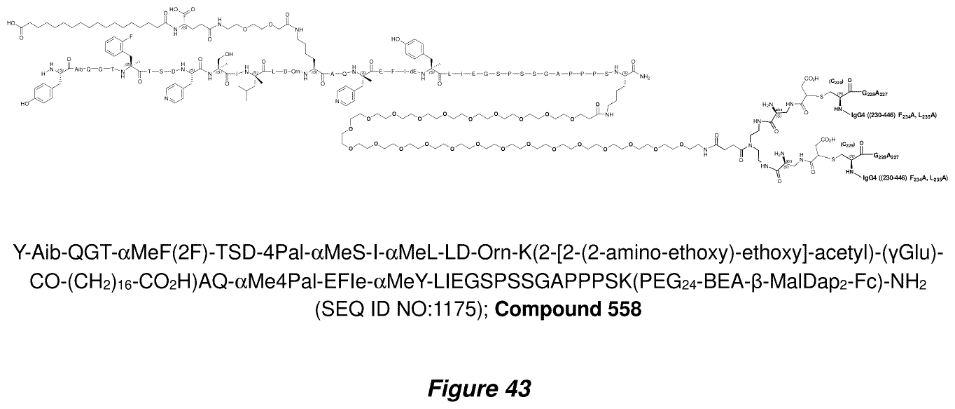

As indicated in Table 3 above, the molecular structure of certain exemplary compounds are provided in FIGS. 1-43.

### In Vitro Functional Activity

Functional activity is determined in GIP-R, GLP-1R, and GcgR-expressing HEK-293 clonal cell lines. Each receptor cell line is treated with example compounds (20 point concentration response curves with 2.75-fold serial dilutions prepared with a Labcyte Echo Acoustic Liquid Handler) in DMEM (Gibco Cat #31053) supplemented with 1×GlutaMAX™ (L-alanyl-L-glutamine dipeptide, Gibco Cat #35050), 0.1% Casein (Sigma Cat #C4765), 1% HSA (Human Serum Albumin, Sigma Cat #A3782) 500 μM IBMX (3-isobutyl-1-methylxanthine) and 20 mM HEPES (4-(2-hydroxyethyl)-1-piperazineethanesulfonic acid) in a 20 μl assay volume.

After a 30-minute incubation at 37° C., the resulting increase in intracellular cAMP is quantitatively determined using the CisBio cAMP Dynamic 2 HTRF Assay Kit (62AM4PEJ). Briefly, cAMP levels within the cell are detected by adding the cAMP-d2 conjugate in cell lysis buffer followed by the antibody anti-cAMP-Eu3+-Cryptate, also in cell lysis buffer. The resulting competitive assay is incubated for at least 60 minutes at room temperature and then detected using a Pherastar Instrument (BMG Labtech) with excitation at 320 nm and emission at 665 nm and 620 nm. Raw data values (emission at 665 nm/620 nm*10,000) are inversely proportional to the amount of cAMP present and were converted to cAMP (nM) per well using a cAMP standard curve.

The amount of cAMP generated (nM) in each well is converted to a percent of the maximal response observed with either human GLP-1₍₇₋₃₆₎NH₂ (SEQ ID NO:1221), human Glucagon (hGcg) (SEQ ID NO:1222), or human GIP₍₁₋₄₂₎NH₂ (SEQ ID NO:1223). A relative EC₅₀ value is derived by non-linear regression analysis using the percent maximal response vs. the concentration of peptide added, fitted to a four-parameter logistic equation.

The geometric mean of relative EC₅₀ data for exemplary dual agonist conjugates, hGcg, hGIP₍₁₋₄₂₎NH₂, and hGLP-1₍₇₋₃₆₎NH₂ and are shown in Table 4 below and exemplary triple agonist conjugates, hGcg, hGIP₍₁₋₄₂₎NH₂, and hGLP-1₍₇₋₃₆₎NH₂ are shown in Table 5 below.

TABLE 4
Functional cAMP potency (Rel EC₅₀ (nM) for exemplary dual agonist therapeutic polypeptide
Fc conjugates and comparators in the presence of 1% HSA and 0.1% Casein.

| | hGcR 1% | hGIPR 1% | hGLP-1R 1% | hGcgR 0.1% | hGIPR 0.1% | hGLP-1R 0.1% |
|---|---|---|---|---|---|---|
| | HSA Rel | HSA Rel | HSA Rel | Casein Rel | Casein Rel | Casein Rel |
| Compound | EC₅₀ (nM) | EC₅₀ (nM) | EC₅₀ (nM) | EC₅₀ (nM) | EC₅₀ (nM) | EC₅₀ (nM) |
| hGcg | 2.5 | >300 | 160 | 2.19 | >300 | 116 |
| hGIP₁₋₄₂ | >9950 (n = | 0.39 | >9950 | 95.7 (n = | 0.467 | >9950 |
| amide | 1/194) | | | 1/219) | | |
| hGLP-1₇₋₃₆ | >99.5 (n = | >99.5 | 0.39 | 77.7 (n = | >99.5 | 0.338 |
| amide | 1/195) | | | 1/232) | | |
| 1 | >1200 (n = | 5.16 | 548.0 | >1200 (n = | 0.032 | 10.60 |
| | 1/16) | | | 1/16) | | |
| 2 | >627 (n = | 1.19 | 247.0 | >627 (n = | 0.027 | 4.81 |
| | 1/2) | | | 1/2) | | |
| 3 | >552 (n = | 4.29 | >552 | >552 (n = | 0.024 | 2.91 |
| | 1/2) | | | 1/2) | | |
| 4 | >672 (n = | 5.20 | >672 | >672 (n = | 0.029 | 8.36 |
| | 1/2) | | | 1/2) | | |
| 5 | >741 (n = | 5.02 | >741 | >741 (n = | 0.025 | 6.24 |
| | 1/2) | | | 1/2) | | |
| 6 | >542 (n = | 2.99 | >542 | >542 (n = | 0.019 | 3.48 |
| | 1/2) | | | 1/2) | | |
| 7 | >677 (n = | 3.97 | 405.0 | >677 (n = | 0.024 | 3.40 |
| | 1/2) | | | 1/2) | | |
| 8 | >383 | 2.22 | >383 | >383 | 0.020 | 4.12 |
| 9 | >602 (n = | 3.54 | 245.0 | >602 (n = | 0.029 | 2.95 |
| | 1/2) | | | 1/2) | | |
| 10 | >532 (n = | 2.30 | 200.0 | >532 (n = | 0.017 | 2.22 |
| | 1/2) | | | 1/2) | | |
| 11 | >801 | 3.74 | >801 | >801 | 0.040 | 4.00 |
| 12 | >980 | 5.68 | >980 | >980 | 0.049 | 3.97 |
| 13 | >980 | 4.02 | 490.0 | >980 | 0.036 | 3.86 |
| 14 | >602 | 2.15 | 281.0 | >602 | 0.028 | 4.40 |
| 15 | >826 (n = | 5.08 | 153.0 | >826 (n = | 0.031 | 0.86 |
| | 1/2) | | | 1/2) | | |
| 16 | >836 (n = | 7.33 | 156.0 | >836 (n = | 0.047 | 0.88 |
| | 1/2) | | | 1/2) | | |
| 17 | >761 (n = | 13.50 | 228.0 | >761 (n = | 0.127 | 1.42 |
| | 1/2) | | | 1/2) | | |
| 18 | >826 (n = | 6.93 | 274.0 | >826 (n = | 0.040 | 1.47 |
| | 1/2) | | | 1/2) | | |
| 19 | >642 (n = | 12.40 | 176.0 | >642 (n = | 0.178 | 0.88 |
| | 1/2) | | | 1/2) | | |
| 20 | >891 (n = | 7.50 | 63.0 | >891 (n = | 0.159 | 0.92 |
| | 1/2) | | | 1/2) | | |
| 21 | >1030 | 7.36 | 71.1 | 111 | 0.076 | 0.62 |
| 22 | >950 | 2.23 | 110.0 | >950 | 0.044 | 1.62 |
| 23 | >935 | 2.00 | 251.0 | >935 | 0.032 | 5.00 |
| 24 | >975 | 3.76 | 399.0 | >975 | 0.036 | 3.36 |
| 25 | >975 | 3.47 | 207.0 | >975 | 0.028 | 2.86 |
| 26 | >1330 | 3.70 | 681.0 | >1330 | 0.031 | 6.41 |
| 27 | >1010 | 5.08 | >1010 | >1010 | 0.037 | 9.34 |
| 28 | >1100 (n = | 2.60 | 203.0 | >1100 (n = | 0.024 | 1.35 |
| | 1/2) | | | 1/2) | | |
| 29 | >1160 (n = | 2.79 | 158.0 | >1160 (n = | 0.024 | 1.26 |
| | 1/2) | | | 1/2) | | |
| 30 | >1100 (n = | 2.30 | 203.0 | >1100 (n = | 0.020 | 1.58 |
| | 1/2) | | | 1/2) | | |
| 31 | >871 | 3.52 | 229.0 | >871 | 0.026 | 1.88 |
| 32 | >1310 (n = | 2.62 | 190.0 | >1310 (n = | 0.097 | 2.74 |
| | 1/17) | | | 1/17) | | |
| 33 | >726 | 2.25 | 113.0 | >726 | 0.042 | 2.11 |
| 34 | >725 | 3.67 | 286.0 | >725 | 0.037 | 2.14 |
| 35 | >735 | 4.41 | 210.0 | >735 | 0.041 | 2.27 |
| 36 | >836 | 5.95 | 332.0 | >836 | 0.051 | 2.74 |
| 37 | >701 | 3.39 | 490.0 | >701 | 0.040 | 2.21 |
| 38 | >632 | 3.51 | 183.0 | >632 | 0.040 | 1.97 |
| 39 | >558 | 2.56 | 125.0 | >558 | 0.032 | 1.92 |
| 40 | >711 | 2.96 | 113.0 | >711 | 0.037 | 0.96 |
| 41 | >443 | 3.21 | 82.8 | >443 | 0.086 | 0.85 |
| 42 | >1110 | 24.00 | 540.0 | >1110 | 0.059 | 1.45 |
| 43 | >1090 | 24.00 | 339.0 | >1090 | 0.124 | 1.31 |
| 44 | >1160 | 5.15 | 53.9 | >1160 | 0.083 | 0.85 |
| 45 | >1030 | 5.35 | 53.2 | >1030 | 0.166 | 0.96 |
| 46 | >915 (n = | 1.68 | 120.0 | >915 (n = | 0.032 | 1.61 |
| | 1/8) | | | 1/8) | | |
| 47 | >1220 (n = | 2.00 | 101.0 | >1220 (n = | 0.027 | 1.03 |
| | 1/9) | | | 1/9) | | |
| 48 | >1130 | 0.85 | 103.0 | >1130 | 0.050 | 2.13 |
| 49 | | 1.07 | ND | | 0.027 | ND |
| 50 | | 1.19 | ND | | 0.019 | 0.78 |
| 51 | >846 | 1.67 | 204.0 | >846 | 0.026 | 1.02 |
| 52 | >871 | 2.43 | 152.0 | >871 | 0.036 | 0.70 |
| 53 | >876 | ND | 118.0 | >876 | ND | 0.73 |
| 54 | >876 | 3.06 | 121.0 | >876 | 0.040 | 0.71 |
| 55 | >537 | 4.48 | 253.0 | >537 | 0.051 | 2.94 |
| 56 | >950 | 3.70 | 317.0 | >950 | 0.041 | 3.57 |
| 57 | >527 | 6.65 | 295.0 | >527 | 0.041 | 3.01 |
| 58 | >1120 | 9.17 | 238.0 | >1120 | 0.038 | 0.73 |
| 59 | >1160 | 9.85 | 241.0 | >1160 | 0.029 | 0.93 |
| 60 | >1110 | 5.80 | 347.0 | >1110 | 0.031 | 1.00 |
| 61 | >1100 | 7.79 | 302.0 | >1100 | 0.047 | 0.92 |
| 62 | >1130 | 13.00 | 310.0 | >1130 | 0.048 | 0.76 |
| 63 | >1350 | 9.01 | 308.0 | >1350 | 0.023 | 0.65 |
| 64 | >1040 | 19.20 | 235.0 | >1040 | 0.059 | 1.05 |
| 65 | >861 | 12.00 | 210.0 | >861 | 0.029 | 0.86 |
| 66 | >955 | 15.20 | 365.0 | >955 | 0.042 | 1.07 |
| 67 | >791 | 11.30 | 336.0 | >791 | 0.038 | 1.37 |
| 68 | >905 | 16.30 | 565.0 | >905 | 0.039 | 1.93 |
| 69 | >856 | 11.10 | 381.0 | >856 | 0.053 | 1.03 |
| 70 | >1020 | 2.71 | 191.0 | >1020 | 0.024 | 1.81 |
| 71 | >1050 | 2.59 | 186.0 | >1050 | 0.042 | 1.03 |
| 72 | >1100 | 2.11 | 177.0 | >1100 | 0.043 | 2.01 |
| 73 | >1220 | 4.58 | 472.0 | >1220 | 0.041 | 1.97 |
| 74 | >1060 | 4.22 | 232.0 | >1060 | 0.032 | 1.26 |
| 75 | >1110 | 3.93 | 254.0 | >1110 | 0.042 | 1.15 |
| 76 | >1180 | 3.39 | 324.0 | >1180 | 0.036 | 1.39 |
| 77 | >871 | 3.95 | 270.0 | >871 | 0.057 | 1.64 |
| 78 | >552 | 4.76 | >552 | >552 | 0.061 | 6.62 |
| 79 | >1020 | 6.00 | >1020 | >1020 | 0.051 | 8.38 |
| 80 | >502 | 10.90 | >502 | >502 | 0.035 | 1.85 |
| 81 | >1020 | 11.40 | 304.0 | >1020 | 0.045 | 1.69 |
| 82 | >970 | 10.50 | 448.0 | >970 | 0.035 | 1.85 |
| 83 | >940 | 6.08 | 360.0 | >940 | 0.049 | 2.83 |
| 84 | >493 | 2.62 | 132.0 | >493 | 0.054 | 1.91 |
| 85 | >940 | 2.47 | 174.0 | >940 | 0.054 | 1.89 |
| 86 | >995 | 13.00 | >995 | >995 | 0.048 | 3.25 |
| 87 | >1120 | 16.60 | >1120 | >1210 | 0.044 | 3.49 |
| 88 | >866 | 5.41 | 459.0 | >866 | 0.050 | 4.52 |
| 89 | >985 | 3.67 | 315.0 | >985 | 0.066 | 6.03 |
| 90 | >413 | 12.50 | >413 | >413 | 0.047 | 1.92 |
| 91 | >980 | 10.20 | 590.0 | >980 | 0.056 | 2.82 |
| 92 | >990 | 8.11 | 644.0 | >990 | 0.038 | 2.41 |
| 93 | >965 | 2.23 | 276.0 | >965 | 0.044 | 3.15 |
| 94 | >746 | 4.37 | 326.0 | >746 | 0.062 | 3.09 |
| 95 | >920 | 2.26 | 377.0 | >920 | 0.037 | 2.45 |
| 96 | >627 | 2.78 | 116.0 | >627 | 0.083 | 1.25 |
| 97 | >1190 (n = | 1.12 | 111.0 | >1190 (n = | 0.047 | 1.00 |
| | 1/5) | | | 1/5) | | |
| 98 | >1150 (n = | 2.26 | 242.0 | >1150 (n = | 0.038 | 2.06 |
| | 1/5) | | | 1/5) | | |
| 99 | >1210 (n = | 4.19 | 146.0 | >1210 (n = | 0.076 | 1.32 |
| | 1/5) | | | 1/5) | | |
| 100 | >771 (n = | 1.99 | 236.0 | >771 (n = | 0.050 | 2.52 |
| | 1/5) | | | 1/5) | | |
| 101 | >672 (n = | 1.43 | 130.0 | >672 (n = | 0.072 | 2.66 |
| | 1/5) | | | 1/5) | | |
| 102 | >1290 (n = | 2.43 | 80.4 | >1290 (n = | 0.102 | 1.86 |
| | 1/3) | | | 1/3) | | |
| 103 | >1080 | 2.21 | 67.0 | 612 | 0.064 | 0.71 |
| 104 | >1220 | 1.88 | 59.1 | >1220 | 0.036 | 0.61 |
| 105 | >1160 | 1.85 | 77.6 | >1160 | 0.060 | 0.93 |
| 106 | >1160 | 1.53 | 70.3 | >1160 | 0.035 | 0.45 |
| 107 | >1120 | 2.31 | 53.1 | >1120 | 0.049 | 0.55 |
| 108 | >1220 | 2.09 | 90.3 | >1220 | 0.057 | 0.58 |
| 109 | >1180 | 0.86 | 28.0 | >1180 | 0.069 | 0.87 |
| 110 | >1160 | 1.23 | 34.9 | >1160 | 0.052 | 0.75 |
| 111 | >1050 | 1.35 | 26.7 | >1050 | 0.079 | 0.78 |
| 112 | >1210 (n = | 1.25 | 42.7 | >1210 (n = | 0.071 | 1.47 |
| | 1/3) | | | 1/3) | | |
| 113 | >1140 (n = | 1.01 | 39.4 | >1140 (n = | 0.078 | 1.00 |
| | 1/3) | | | 1/3) | | |
| 114 | >1000 | 0.98 | 40.9 | 324 | 0.036 | 0.80 |
| 115 | >1160 (n = | 2.99 | 93.0 | >1160 (n = | 0.073 | 1.42 |
| | 1/3) | | | 1/3) | | |
| 116 | >970 (n = | 2.84 | 100.0 | >970 (n = | 0.059 | 1.53 |
| | 1/2) | | | 1/2) | | |
| 117 | >821 (n = | 1.85 | 64.3 | >821 (n = | 0.044 | 0.65 |
| | 1/2) | | | 1/2) | | |
| 118 | >1050 (n = | 1.75 | 70.3 | >1050 (n = | 0.031 | 0.83 |
| | 1/2) | | | 1/2) | | |
| 119 | >925 (n = | 1.17 | 44.8 | >925 (n = | 0.024 | 0.39 |
| | 1/2) | | | 1/2) | | |
| 120 | >1010 (n = | 1.20 | 41.0 | >1010 (n = | 0.044 | 1.08 |
| | 1/2) | | | 1/2) | | |
| 121 | >1100 (n = | 1.43 | 60.8 | >1100 (n = | 0.034 | 0.57 |
| | 1/2) | | | 1/2) | | |
| 122 | >1070 | 0.93 | 41.0 | >1070 | 0.023 | 0.77 |
| 123 | >662 | 2.45 | 80.1 | >662 | 0.032 | 0.36 |
| 124 | >632 (n = | 1.49 | 75.2 | >632 (n = | 0.038 | 0.69 |
| | 1/4) | | | 1/4) | | |
| 125 | >632 | 1.93 | 67.7 | >632 | 0.022 | 0.40 |
| 126 | >856 | 1.13 | 59.3 | >856 | 0.028 | 0.43 |
| 127 | >1080 (n = | 0.89 | 27.2 | >1080 (n = | 0.037 | 0.66 |
| | 1/3) | | | 1/3) | | |
| 128 | >861 (n = | 0.83 | 23.5 | >861 (n = | 0.040 | 0.58 |
| | 1/3) | | | 1/3) | | |
| 129 | >1020 (n = | 1.01 | 35.1 | >1020 (n = | 0.034 | 0.86 |
| | 1/3) | | | 1/3) | | |
| 130 | >945 (n = | 0.90 | 33.6 | >945 (n = | 0.050 | 0.67 |
| | 1/3) | | | 1/3) | | |
| 131 | >940 (n = | 2.40 | 163.0 | >940 (n = | 0.062 | 1.24 |
| | 1/2) | | | 1/2) | | |
| 132 | >1040 (n = | 2.57 | 159.0 | >1040 (n = | 0.062 | 1.66 |
| | 1/2) | | | 1/2) | | |
| 133 | >1010 (n = | 3.58 | 248.0 | >1010 (n = | 0.051 | 2.10 |
| | 1/7) | | | 1/7) | | |
| 134 | >935 (n = | 1.66 | 104.0 | >935 (n = | 0.048 | 0.94 |
| | 1/9) | | | 1/9) | | |
| 135 | >955 | 6.31 | 310.0 | >955 | 0.053 | 1.19 |
| 136 | >1230 (n = | 1.83 | 79.5 | >1230 (n = | 0.042 | 0.93 |
| | 1/7) | | | 1/7) | | |
| 137 | >1090 | 1.86 | 99.5 | >1090 | 0.038 | 1.00 |
| 138 | >1050 | 5.87 | 208.0 | >1050 | 0.052 | 0.78 |
| 139 | >846 (n = | 1.82 | 177.0 | >846 (n = | 0.056 | 2.65 |
| | 1/6) | | | 1/6) | | |
| 140 | >1210 (n = | 1.43 | 123.0 | >1210 (n = | 0.052 | 1.99 |
| | 1/5) | | | 1/5) | | |
| 141 | >1050 (n = | 1.94 | 77.0 | >1050 (n = | 0.064 | 1.98 |
| | 1/2) | | | 1/2) | | |
| 142 | >1100 | 2.53 | 128.0 | >1100 | 0.041 | 1.16 |
| 143 | >1130 | 1.89 | 88.1 | >1130 | 0.061 | 1.90 |
| 144 | >1020 | 1.97 | 111.0 | >1020 | 0.058 | 1.74 |
| 145 | >831 | 2.15 | 82.3 | >831 | 0.062 | 1.28 |
| 146 | >935 | 1.54 | 89.5 | >935 | 0.056 | 0.87 |
| 147 | >1850 (n = | 1.62 | 110.0 | >1850 (n = | 0.033 | 1.05 |
| | 1/5) | | | 1/5) | | |
| 148 | >1160 (n = | 1.55 | 99.0 | >1160 (n = | 0.036 | 0.75 |
| | 1/5) | | | 1/5) | | |
| 149 | >1330 (n = | 1.76 | 91.6 | >1330 (n = | 0.030 | 0.70 |
| | 1/4) | | | 1/4) | | |
| 150 | >1100 (n = | 1.62 | 181.0 | >1100 (n = | 0.033 | 1.80 |
| | 1/4) | | | 1/4) | | |
| 151 | >1620 (n = | 2.41 | 189.0 | >1620 (n = | 0.020 | 1.11 |
| | 1/3) | | | 1/3) | | |
| 152 | >1450 (n = | 2.84 | 228.0 | >1450 (n = | 0.035 | 1.17 |
| | 1/3) | | | 1/3) | | |
| 153 | >1610 (n = | 2.95 | 245.0 | >1610 (n = | 0.038 | 1.51 |
| | 1/3) | | | 1/3) | | |
| 154 | >1410 (n = | 2.18 | 176.0 | >1410 (n = | 0.024 | 0.86 |
| | 1/3) | | | 1/3) | | |
| 155 | >1610 (n = | 2.27 | 203.0 | >1610 (n = | 0.023 | 0.93 |
| | 1/3) | | | 1/3) | | |
| 156 | >1650 (n = | 2.32 | 190.0 | >1650 (n = | 0.031 | 1.19 |
| | 1/3) | | | 1/3) | | |
| 157 | >771 (n = | 1.95 | 284.0 | >771 (n = | 0.024 | 1.61 |
| | 1/4) | | | 1/4) | | |
| 158 | >945 (n = | 1.39 | 106.0 | >945 (n = | 0.030 | 0.79 |
| | 1/6) | | | 1/6) | | |
| 159 | >2380 (n = | 1.08 | 195.0 | >2380 (n = | 0.027 | 1.51 |
| | 1/4) | | | 1/4) | | |
| 160 | >1660 (n = | 2.71 | 326.0 | >1660 (n = | 0.036 | 1.52 |
| | 1/2) | | | 1/3) | | |
| 161 | >1890 (n = | 2.72 | 199.0 | >1890 (n = | 0.035 | 1.44 |
| | 1/4) | | | 1/4) | | |
| 162 | >1340 (n = | 2.74 | 242.0 | >1340 (n = | 0.035 | 1.09 |
| | 1/2) | | | 1/3) | | |
| 163 | >1500 (n = | 2.69 | 242.0 | >1500 (n = | 0.032 | 1.49 |
| | 1/2) | | | 1/2) | | |
| 164 | >1450 (n = | 0.96 | 55.1 | >1450 (n = | 0.043 | 0.76 |
| | 1/7) | | | 1/7) | | |
| 165 | >1570 (n = | 1.47 | 150.0 | >1570 (n = | 0.032 | 2.15 |
| | 1/2) | | | 1/2) | | |
| 166 | >1060 (n = | 2.30 | 293.0 | 106 (se = | 0.028 | 1.97 |
| | 1/3) | | | 7.08, n = 3) | | |
| 167 | >1040 (n = | 1.40 | 125.0 | >1040 (n = | 0.025 | 1.08 |
| | 1/2) | | | 1/2) | | |
| 168 | >1180 (n = | 1.46 | 106.0 | 621 (se = | 0.029 | 1.03 |
| | 1/9) | | | 304, n = 2/9) | | |
| 169 | >1170 (n = | 1.01 | 49.5 | >1170 (n = | 0.027 | 0.60 |
| | 1/6) | | | 1/6) | | |
| 170 | >1660 (n = | 3.05 | 129.0 | >1660 (n = | 0.040 | 0.86 |
| | 1/13) | | | 1/13) | | |
| 171 | >1190 (n = | 2.43 | 122.0 | >1190 (n = | 0.026 | 0.71 |
| | 1/3) | | | 1/3) | | |
| 172 | >1090 (n = | 1.75 | 93.2 | >1090 (n = | 0.038 | 0.94 |
| | 1/13) | | | 1/13) | | |
| 173 | >1040 (n = | 3.00 | 138.0 | >1040 (n = | 0.030 | 0.69 |
| | 1/7) | | | 1/7) | | |
| 174 | >791 (n = | 2.09 | 93.5 | >791 (n = | 0.047 | 0.65 |
| | 1/3) | | | 1/3) | | |
| 175 | >851 (n = | 2.10 | 113.0 | 197 (se = | 0.045 | 1.14 |
| | 1/6) | | | 34.7, n = 5/6) | | |
| 176 | >975 (n = | 1.88 | 97.2 | 470 (se = | 0.040 | 0.86 |
| | 1/6) | | | 164, n = 5/6) | | |
| 177 | >970 (n = | 2.32 | 160.0 | >970 (n = | 0.044 | 1.62 |
| | 1/6) | | | 1/6) | | |
| 178 | >905 (n = | 1.98 | 131.0 | >905 (n = | 0.032 | 1.04 |
| | 1/6) | | | 1/6) | | |
| 179 | >915 (n = | 1.90 | 89.5 | >915 (n = | 0.044 | 0.97 |
| | 1/5) | | | 1/5) | | |
| 180 | >900 (n = | 1.40 | 62.3 | >900 (n = | 0.040 | 0.69 |
| | 1/5) | | | 1/5) | | |
| 181 | >980 (n = | 1.86 | 101.0 | >980 (n = | 0.023 | 0.71 |
| | 1/3) | | | 1/3) | | |
| 182 | >1030 (n = | 1.45 | 93.2 | >1030 (n = | 0.033 | 0.70 |
| | 1/3) | | | 1/3) | | |
| 183 | >348 (n = | 1.00 | 90.9 | >348 (n = | 0.037 | 0.99 |
| | 1/2) | | | 1/2) | | |
| 184 | >786 (n = | 1.73 | 130.0 | >786 (n = | 0.020 | 1.00 |
| | 1/4) | | | 1/4) | | |
| 185 | >1180 (n = | 1.47 | 102.0 | >1180 (n = | 0.025 | 0.73 |
| | 1/4) | | | 1/4) | | |
| 186 | >1140 (n = | 2.29 | 194.0 | >1140 (n = | 0.035 | 1.11 |
| | 1/3) | | | 1/3) | | |
| 187 | >632 (n = | 1.91 | 125.0 | >632 (n = | 0.051 | 1.20 |
| | 1/3) | | | 1/3) | | |
| 188 | >945 (n = | 1.44 | 105.0 | >945 (n = | 0.034 | 0.87 |
| | 1/3) | | | 1/3) | | |
| 189 | >502 (n = | 2.40 | 142.0 | >502 (n = | 0.078 | 1.13 |
| | 1/2) | | | 1/2) | | |
| 190 | >1150 (n = | 1.51 | 119.0 | >1150 (n = | 0.036 | 0.58 |
| | 1/2) | | | 1/2) | | |
| 191 | >1150 (n = | 1.19 | 73.9 | >1150 (n = | 0.035 | 0.69 |
| | 1/2) | | | 1/2) | | |
| 192 | >806 (n = | 1.15 | 76.8 | 59.6 (se = | 0.031 | 0.90 |
| | 1/5) | | | 12.0, n = 5) | | |
| 193 | >995 (n = | 1.17 | 120.0 | 377 (n = | 0.060 | 1.38 |
| | 1/2) | | | 1/2) | | |
| 194 | >1270 (n = | 1.99 | 104.0 | >1270 (n = | 0.045 | 1.06 |
| | 1/4) | | | 1/4) | | |
| 195 | >1180 (n = | 1.41 | 84.0 | >1180 (n = | 0.053 | 1.40 |
| | 1/2) | | | 1/2) | | |
| 196 | >1090 (n = | 1.10 | 55.3 | >1090 (n = | 0.049 | 0.94 |
| | 1/2) | | | 1/2) | | |
| 197 | >1070 (n = | 1.37 | 111.0 | >1070 (n = | 0.042 | 1.08 |
| | 1/3) | | | 1/3) | | |
| 198 | >1010 (n = | 0.89 | 101.0 | 127 (se = | 0.045 | 2.53 |
| | 1/3) | | | 22.1, n = 3) | | |
| 199 | >1060 (n = | 0.82 | 181.0 | >1270 (n = | 0.063 | 3.86 |
| | 1/3) | | | 1/4) | | |
| 200 | >1090 (n = | 1.28 | 64.5 | 881 (se = | 0.074 | 1.57 |
| | 1/2) | | | 80.9, n = 2/3) | | |
| 201 | >1160 (n = | 1.28 | 94.1 | >1160 (n = | 0.110 | 2.10 |
| | 1/2) | | | 1/2) | | |
| 202 | >1160 (n = | 1.17 | 50.9 | >1160 (n = | 0.038 | 0.83 |
| | 1/3) | | | 1/3) | | |
| 203 | >577 (n = | 1.20 | 82.6 | >577 (n = | 0.102 | 0.84 |
| | 1/3) | | | 1/3) | | |
| 204 | >1270 (n = | 1.59 | 69.6 | >1270 (n = | 0.043 | 1.00 |
| | 1/7) | | | 1/7) | | |
| 205 | >1230 (n = | 1.20 | 104.0 | >1230 (n = | 0.044 | 1.13 |
| | 1/3) | | | 1/3) | | |
| 206 | >1490 (n = | 3.04 | 153.0 | >1490 (n = | 0.041 | 1.02 |
| | 1/4) | | | 1/4) | | |
| 207 | >1330 (n = | 2.03 | 111.0 | >1330 (n = | 0.054 | 0.86 |
| | 1/4) | | | 1/4) | | |
| 208 | >498 (n = | 2.05 | 99.2 | >498 (n = | 0.128 | 1.08 |
| | 1/4) | | | 1/4) | | |
| 209 | >1120 (n = | 1.27 | 72.2 | >1120 (n = | 0.186 | 1.01 |
| | 1/4) | | | 1/4) | | |
| 210 | >1190 (n = | 2.42 | 140.0 | >1190 (n = | 0.040 | 1.38 |
| | 1/3) | | | 1/3) | | |
| 211 | >1220 (n = | 1.37 | 96.3 | >1220 (n = | 0.038 | 0.87 |
| | 1/3) | | | 1/3) | | |
| 212 | >1070 (n = | 1.69 | 87.7 | >1070 (n = | 0.045 | 0.98 |
| | 1/4) | | | 1/4) | | |
| 213 | >771 (n = | 1.52 | 66.2 | >771 (n = | 0.073 | 0.95 |
| | 1/4) | | | 1/4) | | |
| 214 | >1480 (n = | 1.06 | 36.7 | >1480 (n = | 0.035 | 0.50 |
| | 1/4) | | | 1/4) | | |
| 215 | >1040 (n = | 1.16 | 68.2 | 87.4 (se = | 0.064 | 1.24 |
| | 1/4) | | | 6.90, n = 4) | | |
| 216 | >1260 (n = | 2.16 | 102.0 | 587 (se = | 0.062 | 1.14 |
| | 1/4) | | | 128, n = 3) | | |
| 217 | >1220 (n = | 12.40 | 58.7 | >1220 (n = | 0.375 | 1.06 |
| | 1/4) | | | 1/4) | | |
| 218 | >846 (n = | 5.83 | 54.4 | >846 (n = | 0.185 | 0.72 |
| | 1/3) | | | 1/3) | | |
| 219 | >1070 (n = | 1.83 | 73.1 | >1070 (n = | 0.057 | 1.51 |
| | 1/3) | | | 1/3) | | |
| 220 | >1030 (n = | 1.39 | 78.5 | >1030 (n = | 0.034 | 1.39 |
| | 1/3) | | | 1/3) | | |
| 221 | >1130 (n = | 1.15 | 78.7 | >1130 (n = | 0.053 | 1.21 |
| | 1/3) | | | 1/3) | | |
| 222 | >1130 (n = | 1.98 | 123.0 | >1130 (n = | 0.039 | 1.24 |
| | 1/2) | | | 1/2) | | |
| 223 | >1200 (n = | 1.38 | 64.9 | >1200 (n = | 0.036 | 0.92 |
| | 1/2) | | | 1/2) | | |
| 224 | >910 (n = | 0.76 | 251.0 | 296 (se = | 0.004 | 3.27 |
| | 1/2) | | | 302, n = 2) | | |
| 225 | >1030 (n = | 0.89 | 126.0 | >1030 (n = | 0.030 | 1.76 |
| | 1/3) | | | 1/3) | | |
| 226 | >697 (n = | 1.02 | 72.1 | 332 (se = | 0.056 | 1.17 |
| | 1/5) | | | 94.4, n = 5) | | |
| 227 | >667 (n = | 1.44 | 128.0 | >667 (n = | 0.038 | 0.92 |
| | 1/5) | | | 1/5) | | |
| 228 | >781 (n = | 1.33 | 89.5 | >781 (n = | 0.045 | 0.89 |
| | 1/4) | | | 1/4) | | |
| 229 | >1290 (n = | 0.86 | 64.5 | >1290 (n = | 0.033 | 0.85 |
| | 1/3) | | | 1/3) | | |
| 230 | >1200 (n = | 0.83 | 104.0 | >1200 (n = | 0.029 | 2.24 |
| | 1/3) | | | 1/3) | | |
| 231 | >1230 (n = | 1.09 | 58.0 | >1230 (n = | 0.038 | 0.67 |
| | 1/3) | | | 1/3) | | |
| 232 | >1130 (n = | 1.62 | 81.3 | >1130 (n = | 0.029 | 0.96 |
| | 1/3) | | | 1/3) | | |
| 233 | >1140 (n = | 1.55 | 283.0 | >1140 (n = | 0.040 | 5.50 |
| | 1/3) | | | 1/3) | | |
| 234 | >1140 (n = | 3.94 | 178.0 | >1140 (n = | 0.042 | 2.25 |
| | 1/3) | | | 1/3) | | |
| 235 | >1100 (n = | 1.77 | 212.0 | >1100 (n = | 0.058 | 3.80 |
| | 1/3) | | | 1/3) | | |
| 236 | >995 (n = | 1.18 | 90.6 | >995 (n = | 0.027 | 1.54 |
| | 1/4) | | | 1/4) | | |
| 237 | >965 (n = | 1.10 | 53.3 | >965 (n = | 0.033 | 0.86 |
| | 1/4) | | | 1/4) | | |
| 238 | >945 (n = | 2.23 | 85.1 | >945 (n = | 0.038 | 1.02 |
| | 1/4) | | | 1/4) | | |
| 239 | >909 (n = | 5.47 | 57.0 | >909 (n = | 0.089 | 0.97 |
| | 1/2) | | | 1/2) | | |
| 480 | >1360 (n = | 1.62 | 360.0 | >1360 (n = | 0.045 | 4.00 |
| | 1/6) | | | 1/6) | | |
| 481 | >1190 (n = | 1.90 | 283.0 | >1190 (n = | 0.051 | 6.93 |
| | 1/2) | | | 1/2) | | |
| 482 | >1360 (n = | 2.61 | 362.0 | >1360 (n = | 0.055 | 5.05 |
| | 1/5) | | | 1/5) | | |
| 243 | >886 (n = | 0.08 | 5.4 | >886 (n = | 0.085 | 4.97 |
| | 1/3) | | | 1/3) | | |
| 244 | >1040 | 0.04 | 6.1 | >1040 | 0.057 | 9.33 |
| 246 | >896 | 5.72 | 391.0 | >896 | 0.031 | 2.79 |
| 247 | >836 | 0.28 | 205.0 | >836 | 0.024 | 15.40 |
| 248 | >547 | 0.77 | 196.0 | >547 | 0.040 | 11.70 |
| 249 | >715 | 3.95 | 131.0 | >715 | 0.046 | 1.09 |
| 250 | >948 | 9.69 | 302.0 | >948 | 0.043 | 1.10 |
| 251 | >1120 | 0.24 | 17.1 | >1120 | 0.042 | 1.46 |
| 253 | >970 (n = | 0.76 | 89.4 | >970 (n = | 0.079 | 2.63 |
| | 1/2) | | | 1/2) | | |
| 254 | >642 (n = | 0.43 | 34.3 | >642 (n = | 0.028 | 1.44 |
| | 1/2) | | | 1/2) | | |
| 255 | >1070 (n = | 0.28 | 33.8 | >1070 (n = | 0.026 | 1.74 |
| | 1/2) | | | 1/2) | | |
| 256 | >871 | 0.32 | 25.7 | >871 | 0.019 | 1.86 |
| 257 | >453 | 0.59 | 38.8 | >453 | 0.032 | 0.67 |
| 258 | >960 | 0.71 | 34.0 | >960 | 0.019 | 0.70 |
| 259 | >1000 | 0.60 | 31.0 | >1000 | 0.021 | 0.59 |
| 260 | >1210 | 0.67 | 45.0 | >1210 | 0.034 | 1.27 |
| 261 | >1110 (n = | 0.52 | 25.6 | >1110 (n = | 0.013 | 0.17 |
| | 1/3) | | | 1/3) | | |
| 40 | >711 | 2.96 | 113.0 | >711 | 0.037 | 0.96 |
| 266 | >1050 (n = | 1.04 | 174.0 | >1050 (n = | 0.016 | 1.83 |
| | 1/16) | | | 1/16) | | |
| 305 | >766 (n = | 2.67 | 230.0 | >766 (n = | 0.048 | 1.20 |
| | 1/3) | | | 1/3) | | |
| 310 | >1180 (n = | 1.80 | 235.0 | >1180 (n = | 0.032 | 2.77 |
| | 1/5) | | | 1/5) | | |
| 311 | >746 (n = | 2.30 | 214.0 | >746 (n = | 0.049 | 1.60 |
| | 1/4) | | | 1/4) | | |
| 312 | >945 (n = | 1.64 | 94.2 | >945 (n = | 0.044 | 0.79 |
| | 1/4) | | | 1/4) | | |
| 313 | >716 (n = | 1.48 | 97.6 | 180 (se = | 0.088 | 0.68 |
| | 1/4) | | | 62.5, n = 4) | | |
| 314 | >771 (n = | 1.91 | 151.0 | >771 (n = | 0.054 | 1.46 |
| | 1/4) | | | 1/4) | | |
| 315 | >577 (n = | 1.17 | 159.0 | >577 (n = | 0.025 | 0.88 |
| | 1/3) | | | 1/3) | | |
| 316 | >493 (n = | 2.78 | 280.0 | >493 (n = | 0.037 | 2.04 |
| | 1/3) | | | 1/3) | | |
| 317 | >378 | 1.65 | 148.0 | >378 | 0.068 | 0.74 |
| 318 | >920 (n = | 1.45 | 125.0 | >920 (n = | 0.027 | 0.75 |
| | 1/3) | | | 1/3) | | |
| 319 | >871 (n = | 0.99 | 58.0 | >871 (n = | 0.027 | 0.47 |
| | 1/3) | | | 1/3) | | |
| 320 | >1020 (n = | 1.30 | 171.0 | >1020 (n = | 0.013 | 0.94 |
| | 1/3) | | | 1/3) | | |
| 321 | >1110 (n = | 1.23 | 103.0 | >1110 (n = | 0.017 | 0.36 |
| | 1/3) | | | 1/3) | | |
| 322 | >791 (n = | 1.17 | 151.0 | >791 (n = | 0.030 | 1.37 |
| | 1/3) | | | 1/3) | | |
| 323 | >557 (n = | 0.77 | 85.8 | >557 (n = | 0.057 | 0.78 |
| | 1/3) | | | 1/3) | | |
| 324 | >602 (n = | 1.30 | 141.0 | >602 (n = | 0.025 | 1.34 |
| | 1/3) | | | 1/3) | | |
| 325 | >1030 (n = | 1.20 | 131.0 | >1030 (n = | 0.036 | 0.66 |
| | 1/3) | | | 1/3) | | |
| 326 | >756 (n = | 1.54 | 223.0 | >756 (n = | 0.061 | 4.41 |
| | 1/4) | | | 1/4) | | |
| 327 | >726 (n = | 1.65 | 293.0 | >726 (n = | 0.062 | 3.32 |
| | 1/3) | | | 1/3) | | |
| 328 | >463 (n = | 1.41 | 204.0 | >463 (n = | 0.017 | 2.10 |
| | 1/3) | | | 1/3) | | |
| 329 | >1290 (n = | 1.34 | 144.0 | >1290 (n = | 0.028 | 2.53 |
| | 1/4) | | | 1/4) | | |
| 488 | >687 | 0.07 | 5.8 | >687 | 0.104 | 5.13 |
| 489 | >1270 (n = | 1.37 | 57.2 | 211 (se = | 0.039 | 0.81 |
| | 1/6) | | | 51.8, n = 6) | | |
| 483 | >1160 (n = | 3.68 | 112.0 | 300 (se = | 0.055 | 0.64 |
| | 1/16) | | | 40.7, n = 11/16) | | |
| 490 | >1040 (n = | 2.23 | 48.2 | 297 (se = | 0.076 | 1.05 |
| | 1/4) | | | 26.4, n = 4) | | |
| 491 | >826 (n = | 1.53 | 61.8 | 689 (se = | 0.130 | 1.08 |
| | 1/4) | | | 104, n = 3/4) | | |
| 492 | >617 (n = | 0.71 | 25.9 | 358 (se = | 0.049 | 0.84 |
| | 1/4) | | | 87.7, n = 4) | | |

TABLE 5
Functional cAMP potency Rel EC₅₀ (nM) for exemplary compounds
and comparators in the presence of 1% HSA and 0.1% Casein

| | hGcR 1% | hGIPR 1% | hGLP-1R 1% | hGcgR 0.1% | hGIPR 0.1% | hGLP-1R 0.1% |
|---|---|---|---|---|---|---|
| | HSA Rel | HSA Rel | HSA Rel | Casein Rel | Casein Rel | Casein Rel |
| Compound | EC₅₀ (nM) | EC₅₀ (nM) | EC₅₀ (nM) | EC₅₀ (nM) | EC₅₀ (nM) | EC₅₀ (nM) |
| hGcg | 2.5 | >300 | 160 | 2.19 | >300 | 116 |
| hGIP₁₋₄₂ | >9950 | 0.390 | >9950 | >99.5 | 0.467 | >9950 |
| amide | | | | | | |
| hGLP-1₇₋₃₆ | >99.5 | >99.5 | 0.39 | >99.5 | >99.5 | 0.338 |
| amide | | | | | | |
| 330 | >498 | 3.79 | 92.9 | 56.90 | 0.134 | 2.88 |
| 331 | >289 | 2.64 | 44.6 | 12.70 | 0.049 | 0.70 |
| 332 | 419 | 9.01 | 86.1 | 19.90 | 0.153 | 2.87 |
| 333 | 217 | 3.51 | 57.7 | 3.03 | 0.041 | 0.71 |
| 336 | 333 | 3.01 | 101.0 | 7.46 | 0.075 | 1.75 |
| 337 | 100 | 1.43 | 57.9 | 2.14 | 0.030 | 0.76 |
| 338 | >1290 | 2.43 | 80.4 | >1290 | 0.102 | 1.86 |
| 339 | 379 | 1.60 | 68.3 | 2.95 | 0.040 | 0.55 |
| 340 | 285 | 2.12 | 64.8 | 2.74 | 0.031 | 0.40 |
| 341 | 325 | 1.62 | 57.7 | 4.11 | 0.018 | 0.53 |
| 342 | 249 | 1.45 | 53.4 | 3.02 | 0.039 | 0.61 |
| 343 | 506 | 2.13 | 74.1 | 5.65 | 0.033 | 0.56 |
| 344 | 540 | 1.60 | 24.3 | 18.10 | 0.134 | 0.90 |
| 345 | 380 | 0.89 | 30.9 | 3.57 | 0.023 | 0.36 |
| 346 | 614 | 2.80 | 26.1 | 16.80 | 0.171 | 0.60 |
| 347 | 408 | 0.83 | 48.0 | 2.59 | 0.004 | 0.14 |
| 348 | 427 | 2.84 | 94.2 | 5.17 | 0.360 | 1.29 |
| 349 | 385 | 1.36 | 75.2 | 3.69 | 0.016 | 0.60 |
| 350 | >18.3 | 1.48 | >18.3 | 2.61 | 0.033 | 0.72 |
| 351 | >17.8 | 1.51 | >17.8 | 4.61 | 0.049 | 1.36 |
| 352 | 308 | 1.39 | 56.2 | 3.97 | 0.037 | 0.68 |
| 353 | >11.1 | 1.42 | 32.4 | 8.40 | 0.106 | 1.96 |
| 354 | 556 | 1.26 | 96.6 | 4.67 | 0.215 | 1.11 |
| 355 | >19.3 | 0.57 | >19.3 | 1.86 | 0.002 | 0.19 |
| 356 | >756 | 1.63 | 96.4 | 2.37 | 0.042 | 1.19 |
| 357 | 374 | 1.60 | 81.8 | 3.25 | 0.022 | 0.70 |
| 358 | 388 | 2.75 | 91.1 | 3.24 | 0.089 | 1.27 |
| 359 | 686 | 3.70 | 143.0 | 6.27 | 0.058 | 1.47 |
| 360 | 608 | 7.16 | 228.0 | 20.30 | 0.284 | 4.35 |
| 361 | 723 | 4.04 | 160.0 | 19.60 | 0.149 | 2.41 |
| 362 | 613 | 2.81 | 111.0 | 13.90 | 0.093 | 1.65 |
| 363 | 695 | 3.15 | 114.0 | 27.30 | 0.093 | 2.18 |
| 364 | 1050 | 8.95 | 198.0 | 22.30 | 0.263 | 3.00 |
| 365 | 148 | 1.63 | 52.8 | 2.73 | 0.035 | 0.50 |
| 366 | 291 | 1.53 | 99.3 | 3.82 | 0.173 | 1.24 |
| 367 | 211 | 1.66 | 105.0 | 1.85 | 0.038 | 0.87 |
| 368 | 207 | 1.14 | 111.0 | 2.52 | 0.056 | 1.07 |
| 369 | 476 | 2.34 | 138.0 | 6.24 | 0.042 | 1.01 |
| 370 | 577 | 1.64 | 117.0 | 9.23 | 0.027 | 1.26 |
| 371 | 996 | 2.11 | 252.0 | 27.70 | 0.033 | 2.45 |
| 372 | 360 | 1.80 | 133.0 | 4.35 | 0.026 | 0.78 |
| 373 | >1330 | 1.20 | 86.6 | 29.30 | 0.024 | 0.70 |
| 374 | 346 | 2.18 | 198.0 | 2.92 | 0.027 | 1.00 |
| 375 | 670 | 2.48 | 167.0 | 6.31 | 0.082 | 1.01 |
| 376 | 631 | 1.84 | 129.0 | 8.02 | 0.064 | 0.81 |
| 377 | 535 | 2.00 | 128.0 | 3.96 | 0.058 | 0.99 |
| 378 | 145 | 4.05 | 89.9 | 1.72 | 0.105 | 0.80 |
| 379 | 512 | 1.23 | 159.0 | 7.82 | 0.010 | 1.05 |
| 380 | 332 | 1.21 | 177.0 | 3.83 | 0.010 | 1.31 |
| 381 | 235 | 1.65 | 233.0 | 2.88 | 0.022 | 2.54 |
| 382 | 151 | 1.33 | 78.6 | 1.65 | 0.043 | 0.82 |
| 383 | 473 | 1.10 | 79.4 | 4.50 | 0.013 | 0.39 |
| 384 | 149 | 0.99 | 52.5 | 2.32 | 0.021 | 0.36 |
| 385 | 499 | 1.19 | 56.9 | 6.99 | 0.016 | 0.41 |
| 386 | 444 | 1.19 | 79.2 | 4.08 | 0.019 | 0.51 |
| 387 | 512 | 2.68 | 112.0 | 7.72 | 0.017 | 0.74 |
| 388 | 464 | 1.17 | 63.5 | 6.70 | 0.021 | 0.73 |
| 389 | 544 | 1.56 | 100.0 | 10.50 | 0.031 | 1.06 |
| 390 | 174 | 1.65 | 88.2 | 1.82 | 0.022 | 0.92 |
| 391 | 494 | 1.93 | 90.3 | 9.43 | 0.030 | 0.95 |
| 392 | 412 | 0.77 | 60.5 | 12.30 | 0.030 | 1.28 |
| 393 | 551 | 1.69 | 97.0 | 19.90 | 0.027 | 2.02 |
| 394 | 162 | 1.21 | 89.6 | 1.81 | 0.012 | 0.53 |
| 395 | 665 | 5.63 | 416.0 | 5.41 | 0.015 | 0.93 |
| 396 | 636 | 3.14 | 230.0 | 3.65 | 0.013 | 0.61 |
| 397 | 456 | 1.29 | 81.4 | 9.01 | 0.013 | 0.41 |
| 398 | 205 | 0.78 | 69.6 | 2.27 | 0.004 | 0.32 |
| 399 | 479 | 1.94 | 90.3 | 13.80 | 0.041 | 1.37 |
| 400 | 284 | 1.66 | 108.0 | 6.77 | 0.033 | 1.44 |
| 401 | 316 | 1.00 | 90.2 | 3.94 | 0.025 | 0.69 |
| 402 | 322 | 0.84 | 67.2 | 6.51 | 0.021 | 0.70 |
| 403 | 317 | 3.08 | 142.0 | 3.60 | 0.029 | 0.86 |
| 404 | 212 | 1.18 | 76.4 | 2.40 | 0.027 | 0.61 |
| 405 | 447 | 4.93 | 136.0 | 10.80 | 0.045 | 1.15 |
| 406 | 259 | 1.76 | 99.0 | 3.35 | 0.082 | 1.01 |
| 407 | 265 | 1.82 | 150.0 | 3.87 | 0.034 | 1.44 |
| 408 | 229 | 1.27 | 125.0 | 2.95 | 0.043 | 1.25 |
| 409 | >1110 | 3.43 | 116.0 | 31.50 | 0.050 | 0.90 |
| 410 | 797 | 2.86 | 97.8 | 18.50 | 0.037 | 1.09 |
| 411 | 856 | 4.09 | 134.0 | 17.00 | 0.049 | 0.82 |
| 412 | 580 | 3.12 | 134.0 | 10.80 | 0.041 | 1.23 |
| 413 | 473 | 3.61 | 94.3 | 11.90 | 0.051 | 1.03 |
| 414 | 558 | 2.56 | 78.1 | 14.20 | 0.035 | 0.93 |
| 415 | 479 | 4.00 | 168.0 | 6.98 | 0.052 | 1.34 |
| 416 | 710 | 1.37 | 71.3 | 19.50 | 0.038 | 0.86 |
| 417 | 462 | 1.56 | 90.3 | 6.69 | 0.036 | 1.05 |
| 418 | 408 | 1.48 | 91.1 | 8.33 | 0.038 | 1.05 |
| 419 | 271 | 1.73 | 86.1 | 4.96 | 0.042 | 1.47 |
| 420 | 453 | 2.53 | 131.0 | 12.80 | 0.049 | 1.76 |
| 421 | 415 | 3.19 | 185.0 | 7.75 | 0.041 | 2.13 |
| 422 | 206 | 2.62 | 117.0 | 1.78 | 0.040 | 0.89 |
| 423 | 516 | 6.80 | 93.1 | 18.00 | 0.166 | 1.96 |
| 424 | 520 | 2.24 | 73.5 | 34.60 | 0.103 | 1.75 |
| 425 | 908 | 12.70 | 155.0 | 17.20 | 0.182 | 1.44 |
| 426 | 568 | 26.20 | 193.0 | 8.21 | 0.203 | 0.67 |
| 427 | 378 | 5.21 | 121.0 | 4.20 | 0.054 | 0.62 |
| 428 | 331 | 5.15 | 166.0 | 2.61 | 0.057 | 0.86 |
| 429 | 351 | 3.81 | 202.0 | 2.88 | 0.030 | 1.14 |
| 430 | 546 | 5.46 | 367.0 | 5.45 | 0.041 | 5.22 |
| 431 | 211 | 3.53 | 176.0 | 1.29 | 0.014 | 0.81 |
| 432 | 290 | 5.49 | 248.0 | 3.18 | 0.093 | 1.69 |
| 433 | 473 | 2.02 | 135.0 | 13.60 | 0.035 | 1.80 |
| 434 | 194 | 1.88 | 81.8 | 1.43 | 0.012 | 0.76 |
| 435 | 343 | 4.27 | 162.0 | 5.31 | 0.084 | 1.35 |
| 436 | 395 | 1.72 | 153.0 | 4.23 | 0.026 | 0.92 |
| 437 | 503 | 3.23 | 166.0 | 8.31 | 0.047 | 1.38 |
| 438 | 224 | 1.65 | 108.0 | 2.82 | 0.028 | 0.77 |
| 439 | 285 | 2.28 | 78.6 | 4.86 | 0.060 | 0.98 |
| 440 | 193 | 1.20 | 91.4 | 1.92 | 0.031 | 0.70 |
| 441 | >1150 | 5.47 | 318.0 | 66.80 | 0.044 | 3.53 |
| 442 | 808 | 3.87 | 202.0 | 23.50 | 0.026 | 1.16 |
| 443 | 726 | 1.61 | 168.0 | 34.20 | 0.027 | 1.49 |
| 444 | 299 | 3.72 | 108.0 | 4.88 | 0.049 | 0.97 |
| 445 | 548 | 5.25 | 242.0 | 22.50 | 0.066 | 2.68 |
| 446 | 332 | 8.30 | 158.0 | 3.91 | 0.053 | 0.76 |
| 447 | >1040 | 31.30 | 370.0 | 42.60 | 0.441 | 5.00 |
| 448 | 521 | 5.34 | 372.0 | 14.50 | 0.069 | 5.19 |
| 449 | >1170 | 8.61 | 554.0 | 49.70 | 0.236 | 22.20 |
| 450 | 317 | 10.80 | 217.0 | 2.45 | 0.123 | 1.45 |
| 453 | 692 | 3.81 | 135.0 | 18.80 | 0.093 | 1.09 |
| 455 | 420 | 8.17 | 122.0 | 9.42 | 0.225 | 1.91 |
| 456 | 289 | 2.65 | 87.3 | 6.13 | 0.045 | 0.95 |
| 457 | 849 | 2.15 | 115.0 | 33.20 | 0.038 | 1.38 |
| 458 | 293 | 1.96 | 61.5 | 3.69 | 0.048 | 0.96 |
| 459 | 518 | 2.72 | 159.0 | 23.90 | 0.090 | 3.28 |
| 460 | 397 | 9.59 | 93.6 | 8.26 | 0.215 | 1.39 |
| 461 | 316 | 9.02 | 154.0 | 3.81 | 0.137 | 1.20 |
| 462 | 595 | 2.57 | 102.0 | 11.70 | 0.052 | 0.97 |
| 463 | 615 | 2.32 | 110.0 | 19.70 | 0.034 | 1.40 |
| 464 | 658 | 6.18 | 296.0 | 6.54 | 0.073 | 1.07 |
| 465 | 433 | 2.29 | 169.0 | 2.62 | 0.034 | 0.93 |
| 467 | 351 | 2.10 | 132.0 | 3.93 | 0.092 | 1.21 |
| 470 | 302 | 3.09 | 134.0 | 9.16 | 0.178 | 2.13 |
| 471 | 499 | 3.53 | 180.0 | 6.58 | 0.184 | 1.85 |
| 472 | 387 | 1.37 | 129.0 | 7.01 | 0.105 | 1.29 |
| 473 | 460 | 2.85 | 198.0 | 4.20 | 0.070 | 1.10 |
| 474 | 297 | 1.18 | 89.7 | 2.17 | 0.058 | 0.45 |
| 475 | 344 | 1.28 | 157.0 | 6.38 | 0.122 | 1.18 |
| 476 | 616 | 1.41 | 191.0 | 7.29 | 0.061 | 1.21 |
| 477 | 342 | 1.74 | 129.0 | 2.25 | 0.027 | 0.54 |
| 478 | 183 | 0.77 | 112.0 | 1.60 (se = | 0.0949 (se = | 1.00 (se = |
| | | | | 0.0144, n = 2) | 0.0103, n = 2) | 0.0964, n = 2) |
| 494 | 240 | 3.75 | 98.8 | 2.67 | 0.062 | 0.73 |
| 495 | 308 | 4.34 | 110.0 | 2.82 | 0.083 | 0.77 |
| 496 | 223 | 4.87 | 96.3 | 2.52 | 0.131 | 0.67 |
| 497 | 234 | 4.74 | 117.0 | 1.99 | 0.050 | 0.88 |
| 498 | 270 | 3.33 | 128.0 | 1.46 | 0.037 | 0.73 |
| 499 | 449 | 4.20 | 129.0 | 4.88 | 0.042 | 1.01 |
| 500 | 1020 | 2.61 | 71.6 | 15.40 | 0.072 | 0.51 |
| 501 | 273 | 1.00 | 56.5 | 0.72 | 0.001 | 0.18 |
| 502 | 204 | 1.50 | 82.0 | 2.16 | 0.043 | 0.93 |
| 503 | 206 | 2.67 | 138.0 | 1.43 | 0.046 | 1.04 |
| 504 | 420 | 3.86 | 144.0 | 5.39 | 0.056 | 1.23 |
| 505 | 441 | 24.90 | 110.0 | 10.10 | 0.434 | 1.13 |
| 506 | >1200 | 17.70 | 186.0 | 34.10 | 0.280 | 1.27 |
| 507 | >1170 | 4.37 | 210.0 | 14.50 | 0.072 | 1.47 |
| 508 | 448 | 4.15 | 167.0 | 2.34 | 0.029 | 0.74 |
| 509 | 473 | 8.09 | 177.0 | 7.62 | 0.168 | 1.37 |
| 510 | 431 | 3.50 | 103.0 | 6.46 | 0.063 | 0.96 |
| 511 | 207 | 6.04 | 161.0 | 1.54 | 0.089 | 0.64 |
| 512 | 259 | 4.63 | 113.0 | 1.37 | 0.107 | 0.34 |
| 513 | 247 | 4.01 | 102.0 | 2.43 | 0.035 | 0.51 |
| 514 | 290 | 3.57 | 114.0 | 2.39 | 0.051 | 0.75 |
| 515 | 314 | 3.65 | 138.0 | 3.84 | 0.034 | 0.90 |
| 516 | 419 | 2.40 | 87.0 | 4.75 | 0.006 | 0.58 |
| 517 | 152 | 2.20 | 118.0 | 1.34 | 0.028 | 0.34 |
| 518 | 241 | 1.57 | 61.9 | 2.12 | 0.005 | 0.26 |
| 519 | 265 | 1.62 | 99.0 | 4.22 | 0.031 | 0.91 |
| 520 | 275 | 3.25 | 51.9 | 5.64 | 0.044 | 0.37 |
| 521 | 284 | 1.08 | 68.4 | 5.10 | 0.024 | 0.72 |
| 522 | 707 | 5.08 | 102.0 | 10.50 | 0.004 | 0.26 |
| 523 | 363 | 3.41 | 102.0 | 5.34 | 0.045 | 0.68 |
| 524 | 334 | 2.02 | 86.4 | 5.22 | 0.025 | 0.76 |
| 525 | 648 | 2.85 | 124.0 | 17.50 | 0.039 | 1.11 |
| 526 | 546 | 6.92 | 122.0 | 15.80 | 0.126 | 0.89 |
| 527 | >1010 | 2.39 | 102.0 | 20.50 | 0.045 | 0.90 |
| 528 | 311 | 4.17 | 91.1 | 4.40 | 0.085 | 0.96 |
| 529 | 193 | 1.45 | 42.9 | 1.08 | 0.019 | 0.23 |
| 530 | 415 | 0.67 | 22.5 | 3.93 | 0.012 | 0.09 |
| 531 | 327 | 1.65 | 48.9 | 5.16 | 0.024 | 0.58 |
| 532 | 141 | 2.14 | 78.5 | 1.62 | 0.035 | 0.76 |
| 533 | 313 | 0.98 | 60.0 | 5.67 | 0.021 | 0.59 |
| 534 | 918 | 1.19 | 43.5 | 45.10 | 0.025 | 0.46 |
| 535 | 487 | 1.68 | 88.6 | 14.90 | 0.028 | 1.38 |
| 536 | 123 | 0.37 | 13.2 | 0.64 | 0.002 | 0.06 |
| 484 | 456 | 3.09 | 108.0 | 7.58 | 0.044 | 0.96 |
| 537 | 268 | 2.43 | 124.0 | 3.32 | 0.025 | 0.74 |
| 485 | 334 | 3.64 | 91.6 | 7.53 | 0.041 | 0.94 |
| 486 | 420 | 1.72 | 72.4 | 8.27 | 0.019 | 1.02 |
| 487 | 664 | 3.87 | 213.0 | 41.00 | 0.174 | 5.57 |
| 538 | 399 | 2.36 | 69.4 | 8.04 | 0.044 | 1.03 |
| 539 | 363 | 2.19 | 57.7 | 12.20 | 0.066 | 0.83 |
| 540 | 197 | 1.53 | 44.5 | 6.60 | 0.064 | 1.15 |
| 541 | 487 | 1.66 | 77.7 | 20.00 | 0.111 | 2.51 |
| 542 | 289 | 2.04 | 61.1 | 6.68 | 0.093 | 1.39 |
| 543 | 397 | 2.09 | 74.1 | 12.00 | 0.094 | 1.23 |
| 544 | 619 | 1.91 | 72.3 | 24.40 | 0.127 | 1.31 |
| 545 | 403 | 2.52 | 78.1 | 48.10 | 0.118 | 1.43 |
| 546 | 360 | 2.56 | 86.9 | 16.00 | 0.077 | 1.62 |
| 486 | 664 | 3.87 | 213 | 41.0 | 0.174 | 5.57 |
| 550 | 280 | 1.31 | 44.2 | 12.90 | 0.018 | 1.17 |
| 551 | 171 | 0.99 | 33.3 | 8.88 | 0.046 | 1.05 |
| 552 | 129 | 1.21 | 31.6 | 3.34 | 0.017 | 0.89 |
| 554 | 790 | 4.24 | 172.0 | 10.60 | 0.163 | 1.65 |
| 555 | 312 | 2.66 | 98.1 | 6.99 | 0.054 | 0.84 |

### Plasma Levels of Example Compounds in Rats

Plasma levels of example compounds were determined in male Sprague Dawley rats. The animals were purchased from Envigo (Indiana, USA) at 8-9 weeks of age. After acclimation, the rats were randomized. Each group (n=5) received one compound at 10 nmol/kg through subcutaneous (sc) injection. Blood samples were serially collected through tail vein at the points in time indicated in each table. The animal studies were approved by Lilly Institutional Animal Care and Use Committee (IACUC).

The plasma was prepared from collected blood samples. Each plasma sample was serially diluted using compound dilution medium and then incubated with HEK293 cells expressing human GLP1R and cAMP-driving luciferase in 96 well plates for 5 hours. The cells were lysed and luminescence signal intensity measured after Bio-Glo (Promega) was added.

For each test compound, a mathematical equation calculating corresponding concentration of a luminescence signal value was established through non-linear regression analysis (Graphpad Prism) of standard dose-luminescence signal curve data. Using the mathematic equation we converted luminescence data showing dose-dependency in each set of serially diluted samples to concentration and averaged the values as plasma concentration of the sample. The data presented in Table 6 are mean±SE of the averaged values from five rats. As seen in Table 6, the tested example compounds remained in plasma for extended periods of time, including in some cases for at least 28 days.

TABLE 6
Plasma levels (nM) of example compounds in rats.

| Compound | 24 h | 72 h | 168 h | 240 h | 312 h | 336 h | 480 h | 504 h | 672 h |
|---|---|---|---|---|---|---|---|---|---|
| 136 | 32.4 ± 3.3 | | 70.2 ± 2.9 | | | 37.2 ± 3.9 | | 18.1 ± 2.6 | 2.6 ± 1.4 |
| 172 | 82.9 ± 7.4 | | 76.0 ± 2.7 | | | 35.4 ± 2.3 | | 21.5 ± 3.1 | 6.8 ± 1.9 |
| 164 | | 44.6 ± 3.3 | 59.5 ± 3.2 | | | 30.8 ± 1.9 | | 20.8 ± 3.1 | 11.5 ± 3.9 |
| 170 | | 51.9 ± 3.2 | 54.0 ± 4.2 | | | 26.8 ± 3.0 | | 17.9 ± 5.1 | 9.3 ± 3.8 |
| 243 | 13.4 ± 2.8 | 12.1 ± 1.0 | 1.3 ± 0.2 | 7.5 ± 2.1 | | 0.3 ± 0.2 | | 0.1 ± 0.1 | 0.1 ± 0 |
| 1 | 73.3 ± 21.1 | 68.6 ± 3.0 | 46.7 ± 5.6 | 100.7 ± 19.6 | | 40.2 ± 4.9 | | 37.2 ± 5.2 | 20.3 ± 5.0 |
| 38 | 39.4 ± 4.5 | 64.7 ± 2.6 | 38.9 ± 1.4 | | | 47.2 ± 3.5 | | 16.8 ± 1.6 | 12.5 ± 1.6 |
| 266 | 25.8 ± 4.1 | 45.9 ± 3.0 | 23.2 ± 2.1 | | | 29.4 ± 2.1 | | 24.0 ± 4.0 | 10.1 ± 2.5 |
| 1 | | 88.7 ± 6.1 | 71.3 ± 4.9 | 96.0 ± 13.1 | | | | | 31.7 ± 2.7 |
| 3 | 70.4 ± 6.9 | 134.4 ± 7.6 | 90.9 ± 7.7 | 49.9 ± 9.1 | | 59.9 ± 8.2 | | 28.7 ± 2.4 | 52.7 ± 7.8 |
| 10 | 63.8 ± 5.2 | 98.4. ± 8.8 | 85.7 ± 6.5 | 42.0 ± 4.3 | | 52.3 ± 5.9 | | 32.2 ± 2.3 | 31.7 ± 6.0 |
| 2 | 51.8 ± 6.3 | 124.1 ± 10.9 | 47.2 ± 3.4 | 53.3 ± 7.2 | | 98.3 ± 10.0 | | 81.0 ± 14.1 | 22.0 ± 2.1 |
| 15 | 85.6 ± 12.6 | 103.8 ± 8.4 | 85.5 ± 4.3 | 46.8 ± 2.0 | | 19.6 ± 6.3 | | 50.2 ± 11.6 | 24.7 ± 3.0 |
| 18 | 88.2 ± 9.8 | 144.7 ± 10.5 | 58.2 ± 5.5 | 41.1 ± 4.1 | | 51.0 ± 7.9 | | 64.4 ± 7.8 | 25.5 ± 2.3 |
| 345 | | 58.1 ± 11.3 | 53.8 ± 11.0 | | | 42.1 ± 10.1 | | | 14.3 ± 3.5 |
| 260 | 37.2 ± 2.7 | | 43.6 ± 1.7 | 38.2 ± 3.5 | | 19.1 ± 1.8 | | 19.7 ± 5.1 | 5.5 ± 2.2 |
| 47 | 46.0 ± 6.1 | | 35.4 ± 2.1 | | 30.2 ± 4.3 | | 10.5 ± 2.4 | | 7.4 ± 2.2 |
| 158 | 33.1 ± 3.3 | | 54.4 ± 1.1 | | 5.3 ± 5.2 | | 0 | | 0 |
| 168 | 31.8 ± 1.4 | | 54.2 ± 5.8 | | 24.9 ± 7.3 | | 1.5 ± 1.3 | | 0 |
| 169 | 30.8 ± 4.0 | | 62.2 ± 5.8 | | 20.0 ± 5.1 | | 0.1 ± 0 | | 0 |
| 185 | 30.8 ± 5.3 | | 64.4 ± 6.7 | | 5.9 ± 4.3 | | 0.5 ± 0.4 | | 0 |
| 188 | 24.9 ± 3.1 | | 40.0 ± 4.4 | | 24.8 ± 5.8 | | 0.1 ± 0.0 | | 0.0 ± 0.0 |
| 147 | | | 72.3 ± 6.1 | | | 35.6 ± 2.5 | | 2.4 ± 2.3 | 1.5 ± 1.5 |
| 183 | | | 95.5 ± 2.4 | | | 24.5 ± 6.9 | | 0.9 ± 0.6 | 0 ± 0 |
| 184 | | | 61.1 ± 10.1 | | | 19.3 ± 9.1 | | 7.1 ± 4.5 | 4.5 ± 2.7 |
| 187 | | | 60.6 ± 3.1 | | | 10.3 ± 3.2 | | 1.8 ± 1.1 | 0.1 ± 0.1 |
| 204 | | | 82.5 ± 7.2 | | | 21.3 ± 2.1 | | 5.2 ± 2.4 | 2.0 ± 1.7 |
| 483 | | 98.1 ± 5.3 | 71.4 ± 2.5 | | | 57.3 ± 7.2 | | 42.1 ± 7.2 | 16.3 ± 2.3 |
| 129 | 24.5 ± 2.7 | | 63.9 ± 7.7 | | | 27.0 ± 1.7 | | 11.4 ± 4.5 | 2.3 ± 1.4 |
| 136 | 32.4 ± 3.3 | | 70.2 ± 2.9 | | | 37.2 ± 3.9 | | 18.1 ± 2.6 | 2.6 ± 1.4 |
| 173 | | 51.9 ± 3.2 | 42.9 ± 3.5 | | | 24.8 ± 1.2 | | 8.6 ± 2.6 | 3.1 ± 1.1 |
| 185 | 30.8 ± 5.3 | | 64.4 ± 6.7 | | 5.9 ± 4.3 | | 0.5 ± 0.4 | | 0 |
| 188 | 24.9 ± 3.2 | | 40.0 ± 4.4 | | 24.8 ± 5.8 | | 0.1 ± 0 | | 0 |
| 480 | 43.0 ± 11.6 | | 45.8 ± 12.0 | | | 29.4 ± 8.1 | | 15.8 ± 4.3 | 10.4 ± 2.8 |
| 467 | 78.6 ± 6.1 | | | | | 9.6 ± 1.3 | | 3.6 ± 0.6 | 1.5 ± 0.4 |
| 387 | 43.4 ± 12.0 | 55.6 ± 12.5 | 46.7 ± 11.2 | 25.9 ± 3.5 | | 15.9 ± 3.4 | | 7.5 ± 2.1 | 3.3 ± 0.7 |
| 403 | | 90.0 ± 4.1 | 53.1 ± 1.0 | | 24.7 ± 1.0 | | | 5.8 ± 1.9 | 1.1 ± 0.4 |
| 413 | | 59.5 ± 5.7 | | 28.0 ± 2.2 | | | | 3.5 ± 0.5 | 3.4 ± 0.6 |
| 433 | | 86.2 ± 4.7 | | 37.3 ± 3.2 | | | | 10.8 ± 1.1 | 9.1 ± 0.9 |
| 435 | | 82.6 ± 2.9 | | 26.0 ± 2.9 | | 19.9 ± 1.8 | | | 1.7 ± 0.4 |
| 444 | | 55.9 ± 4.1 | | 23.3 ± 3.0 | | 11.5 ± 1.5 | | | 0.4 ± 0.2 |
| 456 | | 94.3 ± 5.0 | 58.9 ± 3.1 | | 20.4 ± 1.1 | | | 5.4 ± 0.5 | 1.9 ± 0.3 |
| 484 | 23.7 ± 5.0 | | 69.9 ± 7.9 | | | 28.0 ± 1.6 | | 10.2 ± 2.7 | 2.3 ± 0.2 |
| 485 | | 102.3 ± 12.3 | 66.6 ± 8.8 | | | 27.5 ± 4.0 | | 6.0 ± 1.3 | 3.9 ± 2.4 |

### Pharmacokinetic (PK) Studies

PK studies of certain exemplary compounds are conducted in Cynomolgus monkeys and DIO mice. Plasma concentrations of compounds for these studies are determined using an ELISA method or LC/MS methods, described in more detail below.

### Bioanalytical Methods—ELISA

Plasma concentrations of certain compounds were determined by an ELISA method. Pierce Streptavidin coated plates (Thermo Scientific cat #15500) were coated using biotinylated ELI299 2G5 Antibody (Lilly generated Antibody lot #2G5—22 Aug. 2023). This coating antibody was diluted in Bicarbonate buffer pH 9.6 (Thermo Scientific cat #: 28382) to a final concentration of 1 μg/mL and added to the plate at 100 μl/well and stored overnight at 2-8° C. Following overnight incubation, the plates were then washed using TBS-Tween wash buffer (TEKNOVA Cat #: T0310). All wash steps use the same wash buffer and are carried out by washing the plate 4× using 300 μl/well of wash buffer with a shake step between each wash. Following each wash step, the plate is tapped on a paper towel to remove any excess wash buffer. Following the wash step, the coated and washed plate is then blocked using “Blocking and Assay buffer” for this assay (Casein in PBS buffer with additional Tween-20). Casein buffer—Thermo Scientific cat #37528 was supplemented by adding Tween-20 to a final concentration of 0.05%. This buffer was used as the blocking and assay buffer for the assay. Blocking was done using 200 μl/well of blocking buffer and stored at ambient temperature for approximately 1 hour with shaking at 500 RPM. During blocking, standards, quality control samples, and study samples were processed through MRD (Minimum Required Dilution) of 1:20 (monkey) or 1:50 (mouse) in assay buffer and any additional sample dilutions as needed. After blocking, plate was washed, and the processed samples were then added to the plates at 100 μl/well. The plate with samples is then stored at ambient temperature for approximately 1 hour with shaking at 500 RPM. Detection reagent is Mouse Anti-Human IgG4 Fc-HRP antibody (Southern Biotech cat #9040-05). The detection reagent is diluted in assay buffer to a final concentration of 0.017 μg/mL. Following the sample incubation, the plate is washed and incubated with detection solution at 100 μl/well. The plate with detection reagent is then stored at ambient temperature for approximately 1 hour with shaking at 500 RPM. During detection incubation, the detection substrate solution is prepared by mixing equal parts of KPL TMB Peroxidase Substrate (KPL cat #: 5120-0048) and KPL Peroxidase Substrate solution B (KPL cat #: 5120-0037). Following detection reagent incubation, the plate is washed, and detection substrate is added at 100 μl/well volume and incubated for 7-8 minutes until color develops. The reaction is then stopped using a TMB Stop solution (KPL cat #: 5150-0019). The plate is then read using a plate reader at wavelength 450 nm with a 630 nm correction.

### Bioanalytical Methods—LC/MS

Plasma concentrations of compounds were determined by LC/MS methods. Surrogate peptides for the incretin and Fc subunits of the analyte were used as a measure of plasma concentrations. For each assay, aliquots (95 μL) of diluted study samples, blanks, and standards in 100% cyno plasma were transferred to a PCR plate (Thermo Scientific 0.2 mL Non-Skirted, 96 Well PCR Plates, Cat #AB-0600). The analytes were immunoprecipitated with a biotin-labeled anti-human IgG antibody (Southern Biotech Goat Anti-Human IgG, Monkey ads-BIOT, Cat #2049-08) pre-conjugated to streptavidin-coated magnetic beads (100 μL). The samples, blanks, and standards were washed once with 1×TBST and once with 1×TBS before being eluted in 0.2% formic acid (100 μL). The eluant (95 μL) was transferred to a new plate containing Internal Standards analogous to the incretin and Fc subunits (5 μL of each) and dried under nitrogen on a SPE Dual Dry at 60 degrees Celsius. Once dry, samples, blanks, and standards were reconstituted in 500 mM ammonium carbonate (30 μL) and reduced and alkylated in acetonitrile containing 2% iodoethanol and 1% triethylphosphine (30 μL) for one hour at 40 degrees Celsius, shaking at 600 rpm. Samples, blanks, and standards were dried a second time under nitrogen on an SPE Dual Dry at 60 degrees Celsius before being reconstituted in 50 mM Ammonium Bicarbonate containing 10% methanol and Glu-C Endoproteinase (95 μL) and digested overnight (˜18 hours) at 37 degrees Celsius, shaking at 600 rpm. The reaction was quenched with 88% formic acid (5 μL) and injected for analysis (20 μL) on an Analytical Sales Sprite Armor C18 column (2.1×80 mm, 5 μm). Analyte and Internal Standard surrogate peptides were delivered to a Thermo Fisher Orbitrap Eclipse Tribrid mass spectrometer in 0.1% formic acid in water and 0.1% formic acid in acetonitrile at 400 μL/min. The surrogate peptides were measured by positive mode full scan FT-MS for quantification.

### Pharmacokinetics in Cynomolgus Monkeys

Male Cynomolgus monkeys were administered a single subcutaneous dose (10 nmol/kg) of compound in 40 mM Tris pH 8 Buffer at a volume of 0.5 mL/kg. Blood was collected from each animal at 6, 24, 48, 72, 96, 120, 168, 240, 288, 336, 408, 576, 672, 2352, 3024, and 4200 hours post dose for pharmacokinetic characterization.

Male Cynomolgus monkeys were administered a single intravenous or subcutaneous dose (10 nmol/kg) of compound in 40 mM Tris pH 8 Buffer at a volume of 0.5 mL/kg. Blood was collected from each animal at 6, 24, 48, 72, 96, 168, 240, 288, 336, 408, 480, 576, 672, 840, 1008, 1176, 1344, and 1512 hours post dose for pharmacokinetic characterization.

Female Cynomolgus monkeys were administered a single intravenous or subcutaneous dose (10 nmol/kg) of compound in 40 mM Tris pH 8 Buffer at a volume of 0.5 mL/kg. Blood was collected from each animal at 0.25 (IV only), 6, 24, 48, 72, 96, 168, 240, 288, 336, 408, 480, 576, 672, 840, 1008, 1176, 1344, 1512, 1680, 1848, and 2016 hours post dose for pharmacokinetic characterization.

TABLE 7
Individual and mean pharmacokinetic (PK) parameters following a
single subcutaneous dose to male and female cynomolgus monkeys

| Compound | T_{1/2} | Tmax | Cₘₐₓ | AUC_{0-inf} | CL/F |
|---|---|---|---|---|---|
| (Dose) | (hr) | (hr) | (nmole/L) | (hr*nmole/L) | (mL/hr/kg) |
| Compound 1 | 481 | 168 | 207 | 205347 | 0.049 |
| (10 nmol/kg) | 505 | 168 | 199 | 185833 | 0.054 |
| | 493 | 168 | 203 | 195590 | 0.051 |
| Compound 266 | NC | 240 | 128 | NC | NC |
| (10 nmol/kg) | NC | 72 | 108 | NC | NC |
| | NC | 156 | 118 | NC | NC |
| Compound 32 | NC | 168 | 247 | NC | NC |
| (10 nmol/kg) | NC | 168 | 293 | NC | NC |
| | NC | 168 | 270 | NC | NC |
| Compound 170 | 800 | 336 | 233 | 226000 | 0.044 |
| (10 nmol/kg) | 621 | 96 | 221 | 188000 | 0.053 |
| | 711 | 216 | 227 | 207000 | 0.049 |
| Compound 164 | 421 | 168 | 179 | 139000 | 0.072 |
| (10 nmol/kg) | 507 | 168 | 214 | 190000 | 0.053 |
| | 464 | 168 | 197 | 165000 | 0.062 |
| Compound 172 | 585 | 168 | 186 | 199000 | 0.050 |
| (10 nmol/kg) | 508 | 168 | 256 | 189000 | 0.053 |
| | 547 | 168 | 221 | 194000 | 0.052 |
| Compound 136 | 446 | 240 | 258 | 153000 | 0.065 |
| (10 nmol/kg) | 416 | 288 | 350 | 161000 | 0.062 |
| | 431 | 264 | 304 | 157000 | 0.064 |
| Compound 336 | NC | 48 | 174 | NC | NC |
| (10 nmol/kg) | NC | 120 | 179 | NC | NC |
| | NC | 84 | 177 | NC | NC |
| Compound 387 | 362 | 96 | 93.8 | 65400 | 0.153 |
| (10 nmol/kg) | 315 | 96 | 111 | 46200 | 0.216 |
| | 339 | 96 | 102 | 55800 | 0.185 |
| Compound 456 | 43.6 | 96 | 189 | 81000 | 0.123 |
| (10 nmol/kg) | 93.9 | 96 | 177 | 75500 | 0.132 |
| | 68.8 | 96 | 183 | 78300 | 0.128 |

Abbreviations: AUC_{0-inf} = area under the curve from time 0 hours to infinity; CL/F = clearance/bioavailability; Tₘₐₓ = time to maximum concentration; Cₘₐₓ = maximum observed plasma concentration; T_{1/2} = half-life; NC = not calculated.

TABLE 8
Individual and Mean Pharmacokinetic Parameters Following a Single
Intravenous Dose to Male and Female Cynomolgus Monkeys

| Compound | T_{1/2} | Cₘₐₓ | AUC₀₋ᵢₙₜ | CL |
|---|---|---|---|---|
| (Dose) | (hr) | (nmole/L) | (hr*nmole/L) | (mL/hr/kg) |
| Compound 266 | 501 | 195 | 117000 | 0.086 |
| (10 nmol/kg) | 504 | 143 | 133000 | 0.075 |
| | 503 | 169 | 125000 | 0.080 |
| Compound 336 | 165 | 312 | 133000 | 0.075 |
| (10 nmol/kg) | 29.1 | 322 | 44200 | 0.226 |
| | 97.1 | 317 | 88600 | 0.151 |
| Compound 170 | 306 | 373 | 119000 | 0.084 |
| (10 nmol/kg) | 763 | 449 | 251000 | 0.040 |
| | 535 | 411 | 185000 | 0.062 |
| Compound 164 | 346 | 357 | 128000 | 0.078 |
| (10 nmol/kg) | 439 | 395 | 138000 | 0.073 |
| | 393 | 376 | 133000 | 0.075 |

Abbreviations: AUC_{0-inf} = area under the curve from time 0 hours to infinity, CL = clearance, Cₘₐₓ = maximum observed plasma concentration, T_{1/2} = half-life

As seen in Tables 7-8, the PK profiles in example compounds support a prolonged elimination half-life and decreased clearance compared to currently available incretin therapies. These data demonstrate that the above compounds have a pharmacokinetic profile potentially suitable for once monthly administration.

### Pharmacodynamics in DIO Mice

The in vivo efficacy of certain example compounds on body weight was determined in a diet-induced mouse model of obesity (DIO). Male C57BL/6 DIO mice (The Jackson Laboratory, Bar Harbor, ME) maintained on a calorie-rich diet (containing 40% kcal from fat) were used in the following studies. Mice were individually housed in a temperature-controlled facility (24° C.) with 12-hour light/dark cycle (lights on 22:00) and ad libitum access to food and water. To examine the effect on body weight, obese mice received one subcutaneous dose of vehicle (40 mM Tris-HCl at pH 8.0), or test article (3, 10 and 30 nmol/kg). Body weight and food intake were monitored daily for the first 14 days and then twice weekly for the remainder of the study. Consistent with the desired long duration of action, treatment of obese animals with a single dose of each example compound produced sustained weight loss.

TABLE 9
Body weight change (%) after receiving one subcutaneous dose of each
evaluated compound at the indicated dosages (nmol/kg) in diet-induced
obese mice. Data are presented as mean ± SEM, n = 5 animals
per group. Statistical analysis was performed using a two-way ANOVA,
followed by a Dunnett's method for multiple comparisons.

| | | Day 3 | Day 7 | Day 14 | Day 21 |
|---|---|---|---|---|---|
| | Dose | Weight | Weight | Weight | Weight |
| Compound | (nmol/kg) | Loss (%) | Loss (%) | Loss (%) | Loss (%) |
| 243 | 3 | 10.12 ± 0.71 | 10.92 ± 1.00 | 6.30 ± 0.86 | 3.52 ± 2.06 |
| 243 | 10 | 12.34 ± 0.30 | 14.76 ± 1.19 | 9.68 ± 1.91 | 4.44 ± 1.68 |
| 1 | 10 | 5.76 ± 0.84 | 10.12 ± 0.84 | 10.28 ± 1.28 | 7.72 ± 1.77 |
| 1 | 30 | 9.38 ± 0.60 | 11.12 ± 1.19 | 9.82 ± 1.27 | 8.76 ± 0.96 |
| 2 | 10 | 7.42 ± 0.47 | 13.58 ± 0.92 | 15.08 ± 0.95 | 12.28 ± 1.13 |
| 3 | 10 | 6.52 ± 0.72 | 10.82 ± 0.82 | 12.90 ± 0.55 | 9.52 ± 0.42 |
| 10 | 10 | 8.10 ± 0.80 | 13.68 ± 1.47 | 14.98 ± 0.95 | 11.34 ± 1.04 |
| 1 | 10 | 7.76 ± 1.16 | 9.82 ± 1.16 | 11.00 ± 2.60 | 5.94 ± 2.57 |
| 8 | 10 | 5.00 ± 1.09 | 5.28 ± 1.47 | 5.66 ± 1.80 | 0.58 ± 1.46 |
| 12 | 10 | 6.44 ± 1.16 | 8.34 ± 1.59 | 7.76 ± 1.97 | 2.30 ± 2.45 |
| 13 | 10 | 8.6 ± 0.54 | 11.32 ± 1.40 | 11.75 ± 1.11 | 6.67 ± 1.24 |
| 14 | 10 | 7.34 ± 0.92 | 8.54 ± 1.19 | 8.16 ± 1.55 | 3.04 ± 2.16 |
| 15 | 10 | 9.88 ± 0.77 | 14.02 ± 2.07 | 11.14 ± 1.20 | 4.44 ± 1.11 |
| 16 | 10 | 9.72 ± 0.51 | 13.42 ± 1.57 | 12.1 ± 1.22 | 7.04 ± 1.00 |
| 18 | 10 | 9.68 ± 0.75 | 13.40 ± 2.58 | 12.98 ± 3.22 | 6.84 ± 2.50 |
| 19 | 10 | 9.66 ± 0.61 | 13.08 ± 2.38 | 12.70 ± 1.58 | 6.50 ± 0.86 |
| 20 | 10 | 9.66 ± 0.68 | 12.82 ± 0.94 | 12.02 ± 1.19 | 5.78 ± 1.06 |
| 22 | 10 | 11.42 ± 0.54 | 18.68 ± 1.10 | 19.62 ± 1.03 | 13.70 ± 3.07 |
| 23 | 10 | 7.60 ± 0.75 | 9.40 ± 0.43 | 8.54 ± 0.92 | 3.34 ± 1.71 |
| 28 | 10 | 8.52 ± 1.14 | 11.14 ± 1.52 | 11.20 ± 1.61 | 6.16 ± 2.04 |
| 29 | 10 | 8.52 ± 0.64 | 12.32 ± 1.38 | 12.04 ± 2.13 | 6.04 ± 1.79 |
| 30 | 10 | 7.48 ± 0.98 | 9.88 ± 1.17 | 10.06 ± 1.38 | 5.02 ± 1.38 |
| 31 | 10 | 7.80 ± 0.89 | 9.76 ± 0.97 | 9.22 ± 1.84 | 4.34 ± 2.15 |
| 38 | 10 | 6.66 ± 0.99 | 9.06 ± 1.47 | 7.78 ± 1.71 | 1.52 ± 1.66 |
| 1 | 10 | 2.62 ± 1.30 | 3.26 ± 1.37 | 3.88 ± 1.36 | 2.54 ± 1.60 |
| 1 | 30 | 8.40 ± 0.87 | 10.04 ± 1.73 | 12.26 ± 2.25 | 13.5 ± 3.28 |
| 32 | 10 | 10.88 ± 0.88 | 18.70 ± 2.98 | 22.06 ± 3.87 | 20.34 ± 3.22 |
| 33 | 10 | 8.24 ± 1.09 | 12.52 ± 2.42 | 13.38 ± 3.05 | 10.18 ± 3.30 |
| 40 | 10 | 8.78 ± 1.28 | 12.96 ± 2.39 | 14.18 ± 2.95 | 11.06 ± 3.23 |
| 52 | 10 | 9.88 ± 0.73 | 14.70 ± 2.83 | 14.26 ± 2.77 | 11.56 ± 2.24 |
| 1 | 30 | 8.63 ± 0.59 | 10.28 ± 1.04 | 8.96 ± 1.06 | 6.42 ± 1.48 |
| 46 | 10 | 13.16 ± 1.02 | 27.08 ± 2.68 | 27.52 ± 3.11 | 19.60 ± 2.06 |
| 47 | 10 | 9.24 ± 0.45 | 24.12 ± 0.33 | 27.76 ± 2.03 | 21.06 ± 2.07 |
| 48 | 10 | 10.44 ± 0.92 | 20.52 ± 2.89 | 22.84 ± 3.74 | 14.22 ± 3.96 |
| 260 | 10 | 6.94 ± 1.12 | 14.00 ± 4.38 | 16.52 ± 6.24 | 10.90 ± 4.43 |
| 251 | 10 | 12.80 ± 0.93 | 22.18 ± 2.77 | 22.76 ± 1.83 | 14.86 ± 3.16 |
| 97 | 10 | 13.70 ± 0.51 | 27.48 ± 2.04 | 26.48 ± 1.48 | 18.30 ± 0.92 |
| 98 | 10 | 13.48 ± 0.72 | 28.14 ± 0.47 | 30.7 ± 2.55 | 22.40 ± 2.88 |
| 99 | 10 | 13.38 ± 0.33 | 27.38 ± 1.88 | 27.34 ± 2.45 | 19.56 ± 2.04 |
| 1 | 30 | 8.62 ± 0.59 | 10.28 ± 10.04 | 8.96 ± 1.06 | 6.42 ± 1.48 |
| 32 | 3 | 4.56 ± 0.89 | 7.78 ± 1.06 | 9.66 ± 0.84 | 6.86 ± 2.38 |
| 32 | 10 | 8.22 ± 0.35 | 20.16 ± 1.90 | 21.36 ± 2.42 | 16.58 ± 1.95 |
| 266 | 3 | 4.16 ± .74 | 9.46 ± 1.36 | 10.78 ± 0.97 | 10.14 ± 0.97 |
| 266 | 10 | 8.14 ± 0.82 | 19.76 ± 2.17 | 24.32 ± 3.60 | 18.60 ± 2.07 |
| 47 | 1 | 6.06 ± 0.55 | 8.92 ± 1.08 | 7.48 ± 0.93 | 5.76 ± 1.54 |
| 97 | 1 | 5.04 ± 0.54 | 10.00 ± 2.07 | 8.68 ± 2.36 | 7.02 ± 2.33 |
| 32 | 3 | 5.52 ± 0.44 | 8.74 ± 0.35 | 11.99 ± 1.38 | 10.93 ± 1.59 |
| 310 | 3 | 7.04 ± 0.58 | 12.71 ± 1.86 | 12.81 ± 1.48 | 10.72 ± 1.85 |
| 311 | 3 | 7.30 ± 0.49 | 12.94 ± 0.79 | 14.35 ± 1.10 | 10.47 ± 1.28 |
| 147 | 3 | 8.62 ± 0.33 | 12.56 ± 0.60 | 14.48 ± 1.18 | 9.77 ± 1.73 |
| 312 | 3 | 8.02 ± 0.21 | 15.29 ± 1.81 | 17.50 ± 1.47 | 13.07 ± 1.00 |
| 313 | 3 | 9.02 ± 0.28 | 17.14 ± 0.63 | 19.42 ± 0.96 | 14.46 ± 1.29 |
| 161 | 3 | 5.86 ± 0.65 | 9.92 ± 1.29 | 12.10 ± 1.23 | 8.72 ± 1.83 |
| 314 | 3 | 6.48 ± 0.30 | 9.27 ± 1.03 | 10.91 ± 1.27 | 7.45 ± 1.41 |
| 100 | 10 | 7.12 ± 0.71 | 14.30 ± 1.96 | 15.66 ± 3.28 | 11.88 ± 2.66 |
| 132 | 10 | 10.94 ± 0.69 | 13.68 ± 0.91 | 11.40 ± 1.57 | 6.34 ± 0.97 |
| 133 | 3 | 10.86 ± 0.68 | 14.5 ± 1.15 | 13.12 ± 2.16 | 6.42 ± 2.00 |
| 133 | 10 | 11.62 ± 0.62 | 20.14 ± 1.67 | 19.74 ± 1.41 | 12.90 ± 0.44 |
| 139 | 10 | 11.70 ± 0.46 | 16.78 ± 1.38 | 15.98 ± 1.05 | 9.98 ± 1.36 |
| 147 | 10 | 13.38 ± 0.25 | 23.44 ± 0.81 | 25.70 ± 1.44 | 18.74 ± 1.15 |
| 148 | 3 | 11.24 ± 0.67 | 13.06 ± 0.56 | 12.12 ± 0.95 | 6.00 ± 0.67 |
| 148 | 10 | 13.36 ± 0.18 | 22.72 ± 1.06 | 21.40 ± 1.78 | 13.64 ± 1.32 |
| 305 | 3 | 10.60 ± 0.63 | 13.22 ± 0.83 | 10.84 ± 0.86 | 5.54 ± 1.00 |
| 305 | 10 | 11.30 ± 0.25 | 15.78 ± 0.95 | 13.72 ± 0.86 | 7.88 ± 0.93 |
| 481 | 10 | 10.98 ± 0.64 | 13.32 ± 0.32 | 10.44 ± 0.93 | 6.50 ± 0.55 |
| 32 | 10 | 11.99 ± 0.85 | 24.61 ± 3.20 | 25.59 ± 2.83 | 19.19 ± 2.11 |
| 46 | 3 | 6.67 ± 0.91 | 9.72 ± 0.89 | 10.74 ± 0.68 | 8.67 ± 0.95 |
| 47 | 3 | 9.22 ± 0.84 | 12.83 ± 1.06 | 12.74 ± 0.93 | 8.52 ± 1.38 |
| 98 | 3 | 8.33 ± 0.66 | 11.19 ± 0.70 | 12.72 ± 1.27 | 9.23 ± 1.19 |
| 101 | 3 | 9.08 ± 0.71 | 13.33 ± 1.41 | 13.33 ± 1.14 | 9.96 ± 0.43 |
| 167 | 3 | 10.26 ± 0.66 | 14.52 ± 1.01 | 14.31 ± 1.56 | 9.08 ± 2.52 |
| 181 | 3 | 9.82 ± 0.84 | 15.99 ± 2.90 | 12.55 ± 3.86 | 8.02 ± 3.67 |
| 183 | 3 | 10.40 ± 0.95 | 15.87 ± 2.89 | 16.01 ± 1.95 | 11.24 ± 0.94 |
| 184 | 3 | 8.64 ± 0.94 | 11.92 ± 1.30 | 13.16 ± 1.83 | 8.79 ± 2.43 |
| 185 | 3 | 10.76 ± 0.82 | 16.28 ± 1.28 | 19.70 ± 1.90 | 15.48 ± 2.39 |
| 186 | 3 | 9.84 ± 0.33 | 14.56 ± 0.77 | 14.98 ± 0.82 | 10.76 ± 0.43 |
| 187 | 3 | 10.51 ± 0.50 | 15.19 ± 2.74 | 16.54 ± 3.51 | 11.04 ± 3.09 |
| 188 | 3 | 10.48 ± 0.52 | 17.94 ± 3.13 | 20.31 ± 3.37 | 15.02 ± 2.63 |
| 189 | 3 | 9.80 ± 0.13 | 14.48 ± 0.77 | 15.17 ± 1.96 | 10.22 ± 2.70 |
| 190 | 3 | 11.76 ± 0.51 | 20.56 ± 2.64 | 19.00 ± 2.42 | 11.64 ± 2.29 |
| 191 | 3 | 11.32 ± 0.62 | 19.45 ± 2.84 | 21.66 ± 1.85 | 14.43 ± 1.14 |
| 124 | 3 | 10.42 ± 0.73 | 14.95 ± 1.61 | 15.15 ± 2.25 | 10.47 ± 2.83 |
| 134 | 3 | 10.38 ± 0.51 | 14.42 ± 1.37 | 14.22 ± 1.27 | 8.34 ± 1.53 |
| 149 | 3 | 9.17 ± 0.80 | 13.51 ± 2.21 | 13.81 ± 2.70 | 7.51 ± 2.25 |
| 158 | 3 | 10.18 ± 0.69 | 14.32 ± 1.13 | 14.87 ± 0.63 | 10.14 ± 1.31 |
| 164 | 3 | 10.61 ± 0.78 | 16.34 ± 1.91 | 17.46 ± 1.99 | 11.20 ± 1.07 |
| 168 | 3 | 9.95 ± 1.19 | 14.28 ± 1.54 | 14.96 ± 1.54 | 9.12 ± 1.47 |
| 169 | 3 | 11.47 ± 0.85 | 20.63 ± 2.88 | 20.39 ± 2.06 | 12.83 ± 2.32 |
| 170 | 3 | 9.93 ± 0.62 | 13.88 ± 1.11 | 14.53 ± 0.88 | 9.00 ± 1.70 |
| 173 | 3 | 11.49 ± 0.56 | 15.40 ± 0.84 | 15.24 ± 1.26 | 8.99 ± 1.52 |
| 188 | 3 | 9.46 ± 0.61 | 14.70 ± 1.58 | 16.41 ± 1.18 | 13.31 ± 1.13 |
| 194 | 3 | 8.30 ± 0.99 | 11.46 ± 1.04 | 12.56 ± 1.34 | 9.19 ± 1.34 |
| 204 | 3 | 11.71 ± 0.25 | 16.57 ± 0.98 | 17.38 ± 1.19 | 12.41 ± 1.09 |
| 205 | 3 | 9.49 ± 1.21 | 15.40 ± 3.48 | 15.74 ± 3.93 | 10.00 ± 3.99 |
| 47 | 3 | 7.25 ± 0.74 | 9.61 ± 1.13 | 9.95 ± 1.85 | 4.66 ± 1.41 |
| 127 | 3 | 8.52 ± 0.60 | 12.80 ± 2.54 | 11.84 ± 3.94 | 6.58 ± 3.94 |
| 129 | 3 | 9.05 ± 0.45 | 12.08 ± 0.86 | 11.72 ± 1.19 | 6.30 ± 2.38 |
| 136 | 3 | 10.65 ± 0.59 | 16.56 ± 1.83 | 17.49 ± 1.92 | 12.94 ± 2.60 |
| 170 | 3 | 9.23 ± 0.94 | 16.86 ± 2.14 | 18.65 ± 2.18 | 13.18 ± 2.01 |
| 172 | 3 | 8.96 ± 1.09 | 14.83 ± 2.43 | 15.59 ± 2.58 | 9.66 ± 2.18 |
| 173 | 3 | 7.32 ± 0.48 | 12.28 ± 2.13 | 10.73 ± 2.42 | 8.24 ± 2.71 |
| 332 | 10 | 11.98 ± 0.58 | 25.92 ± 1.82 | 26.16 ± 1.79 | 20.60 ± 1.80 |
| 333 | 10 | 15.28 ± 0.85 | 31.38 ± 2.48 | 25.32 ± 2.46 | 15.80 ± 2.22 |
| 336 | 10 | 12.72 ± 0.65 | 28.04 ± 1.60 | 25.80 ± 2.14 | 20.42 ± 1.40 |
| 101 | 10 | 9.0 ± 0.42 | 21.50 ± 0.95 | 28.60 ± 2.88 | 24.36 ± 2.22 |
| 47 | 1 | 6.06 ± 0.55 | 8.92 ± 1.08 | 7.48 ± 0.93 | 5.76 ± 1.54 |
| 336 | 3 | 5.92 ± 1.17 | 10.13 ± 3.39 | 10.92 ± 3.74 | 8.73 ± 2.70 |
| 467 | 3 | 5.74 ± 0.88 | 10.63 ± 1.61 | 10.03 ± 1.65 | 7.59 ± 1.56 |
| 471 | 3 | 5.84 ± 0.41 | 10.54 ± 0.87 | 10.61 ± 0.77 | 6.37 ± 1.19 |
| 369 | 3 | 6.92 ± 0.49 | 10.67 ± 0.79 | 11.92 ± 0.37 | 9.33 ± 0.40 |
| 370 | 3 | 4.76 ± 0.68 | 7.99 ± 1.49 | 9.00 ± 1.94 | 5.53 ± 1.42 |
| 472 | 3 | 5.00 ± 0.33 | 7.70 ± 1.03 | 8.52 ± 0.91 | 7.41 ± 0.98 |
| 330 | 10 | 8.18 ± 0.52 | 9.48 ± 1.32 | 8.48 ± 1.81 | 8.00 ± 1.94 |
| 330 | 30 | 12.80 ± 0.48 | 20.64 ± 1.83 | 20.74 ± 2.75 | 15.88 ± 2.42 |
| 332 | 10 | 11.98 ± 0.52 | 25.92 ± 1.63 | 26.16 ± 1.60 | 20.6 ± 1.61 |
| 333 | 10 | 15.28 ± 0.76 | 31.38 ± 2.21 | 25.32 ± 2.2 | 15.8 ± 1.98 |
| 387 | 10 | 10.10 ± 0.47 | 12.95 ± 2.19 | 11.19 ± 2.91 | 5.59 ± 1.64 |
| 391 | 3 | 10.51 ± 0.55 | 12.7 ± 0.99 | 8.89 ± 1.27 | 5.29 ± 0.84 |
| 403 | 3 | 11.65 ± 0.57 | 16.41 ± 1.68 | 10.00 ± 1.50 | 5.35 ± 1.44 |
| 413 | 3 | 8.58 ± 0.97 | 8.11 ± 1.32 | 3.94 ± 1.02 | 1.00 ± 0.47 |
| 414 | 3 | 7.86 ± 0.74 | 7.82 ± 0.55 | 5.45 ± 0.33 | 2.72 ± 0.32 |
| 444 | 3 | 8.60 ± 0.80 | 8.93 ± 1.11 | 5.81 ± 0.96 | 3.20 ± 0.96 |
| 359 | 3 | 10.18 ± 0.69 | 11.72 ± 2.39 | 7.12 ± 2.62 | 3.94 ± 2.98 |
| 369 | 3 | 9.24 ± 0.78 | 10.46 ± 1.49 | 5.48 ± 1.17 | 2.20 ± 0.63 |
| 345 | 3 | 12.52 ± 0.81 | 16.68 ± 1.84 | 13.65 ± 1.99 | 10.17 ± 2.12 |
| 454 | 3 | 10.69 ± 0.67 | 14.25 ± 2.06 | 12.41 ± 2.06 | 6.93 ± 2.02 |
| 456 | 3 | 11.51 ± 0.82 | 15.32 ± 0.81 | 10.93 ± 0.46 | 6.22 ± 0.80 |
| 458 | 3 | 11.43 ± 1.38 | 18.42 ± 3.85 | 13.84 ± 3.82 | 9.40 ± 2.60 |
| 462 | 3 | 10.31 ± 0.41 | 13.69 ± 0.35 | 12.95 ± 0.50 | 10.9 ± 0.84 |
| 433 | 3 | 7.04 ± 0.43 | 8.20 ± 0.56 | 7.73 ± 1.46 | 5.82 ± 2.19 |
| 483 | 10 | — | 7.45 ± 0.94 | 19.64 ± 2.05 | 14.11 ± 1.49 |
| 484 | 10 | — | 10.68 ± 1.34 | 23.36 ± 1.34 | 17.01 ± 2.68 |
| 485 | 10 | — | 12.67 ± 0.47 | 27.23 ± 2.52 | 17.52 ± 2.65 |

### Body Weight Dose-Response Studies in Obese Mice

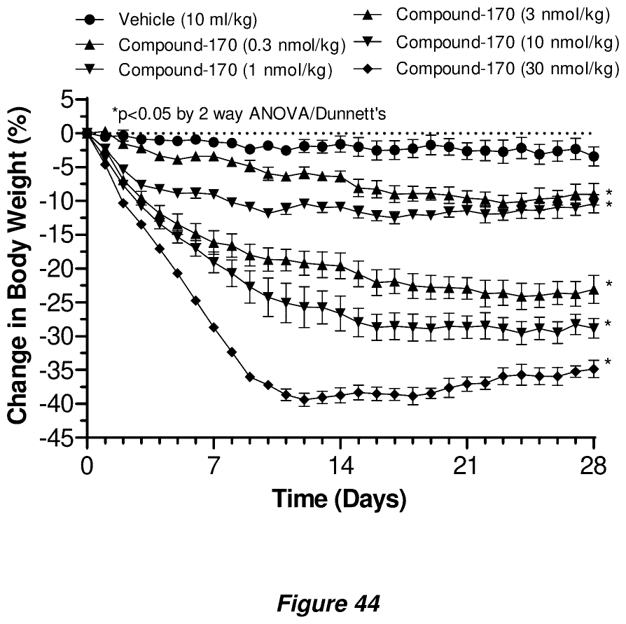

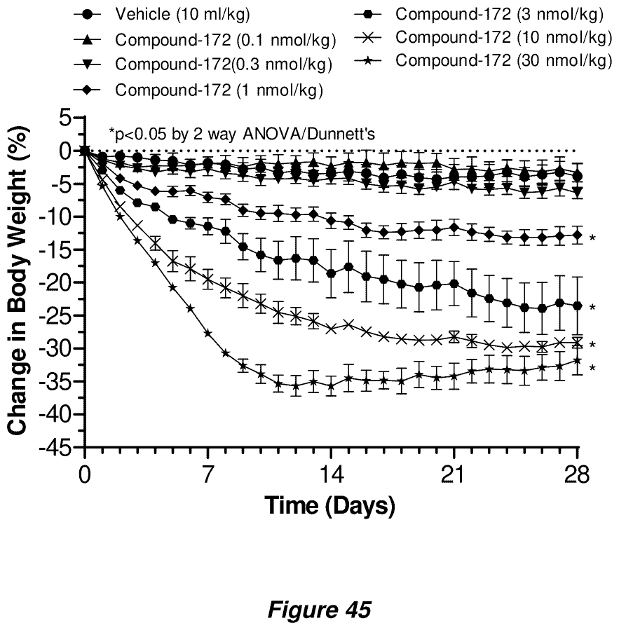

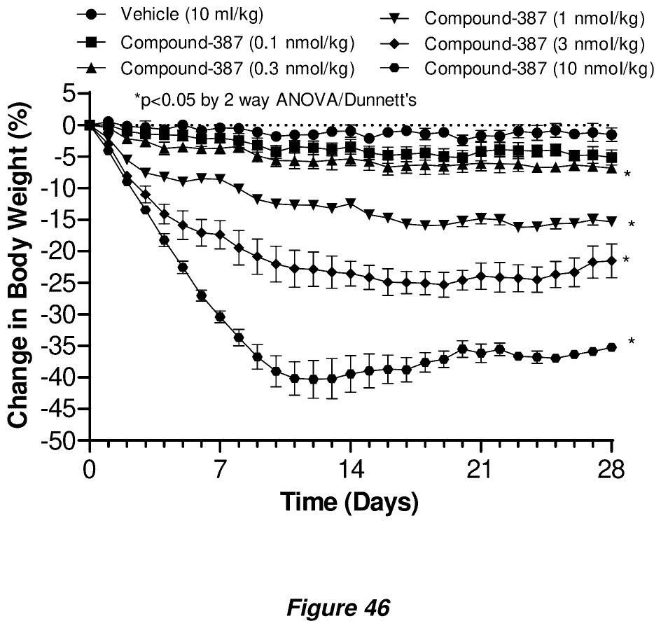

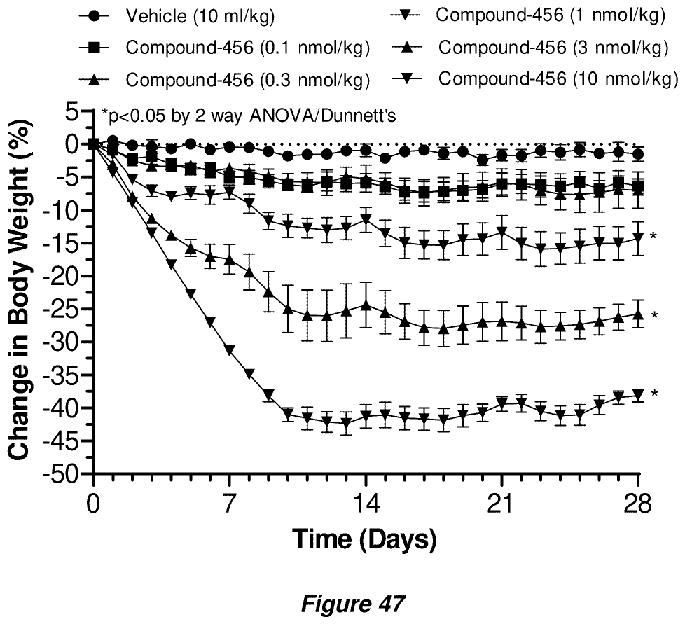

The in vivo efficacy of example compounds on body weight and food intake was determined in a diet-induced mouse model of obesity (DIO). Male C57BL/6 obese mice were maintained on a calorie-rich diet (containing 40% kcal from fat) and were housed in a temperature-controlled facility (24° C.) with 12-hour light/dark cycle (lights on 22:00) and given ad libitum access to food and water. To examine the dose-dependent effects of Compounds 170, 172, 387, and 456 on body weight, obese animals received a weekly subcutaneous dose of vehicle (40 mM Tris-HCl at pH 8.0), or the test articles (0.3, 1, 3, 10 and 10 nmol/kg). Treatment of obese mice with the test articles dose-dependently reduced body weight when compared to control animals (FIGS. 44-47). Values are presented as mean±SEM, n=6 per group. Statistical analysis was performed using a two-way ANOVA, followed by Dunnett's method for multiple comparisons. Significant differences were identified at p<0.05. Together, these findings indicate that the example compounds provide robust weight loss.

1. A compound of the formula: 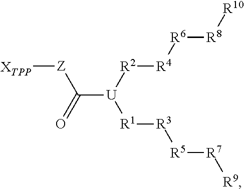
or a pharmaceutically acceptable salt thereof, wherein:
X_{TPP} is a therapeutic polypeptide conjugated to a fatty acid moiety;
Z comprises O, C(O), NH, C₁ to C₃₀ alkyl, (OCH₂CH₂)ₘ, [C(O)NH—CH₂CH₂OCH₂]ₘ, (2-[2-(2-amino-ethoxy)-ethoxy]-acetyl)ₘ, an amino acid, a peptide, or a combination thereof, wherein m is an integer between 1-30;
U comprises C₁ to C₅ alkyl, N, phenyl, or phenyl carbonyl, or a combination thereof;
R¹ and R² are independently absent, a covalent bond, or C₁ to C₅ alkyl;
R³ and R⁴ are independently absent or an amide;
R⁵ and R⁶ are independently absent or R⁵ and R⁶ independently comprise C₁ to C₅ alkyl, phenyl, CH₂O(CH₂CH₂O)ₙ, or a combination thereof, wherein n is an integer between 1-5, wherein C₁ to C₅ alkyl is optionally substituted with one or more of NH₂, CH₂NH₂, and CH₂CH₂NH₂; and
R⁷ and R⁸ are independently absent or selected from the group consisting of: 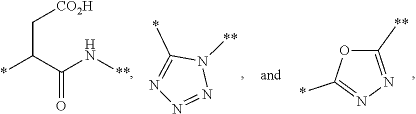
wherein (*) comprises a connection point to R⁹ or R¹⁰ and (**) comprises a connection point to R⁵ or R⁶; and
R⁹ and R¹⁰ are independently an antibody fragment comprising an Fc region.

2. The compound of claim 1 or a pharmaceutically acceptable salt thereof, wherein R⁷ and R⁸ are:

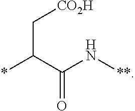

3. The compound of claim 2 or a pharmaceutically acceptable salt thereof, wherein:
R⁵ and R⁶ are each a C₂ alkyl substituted with NH₂;
R³ and R⁴ are each an amide;
R¹ and R² are each a C₂ alkyl; and
U is N or a phenyl.

4. The compound of claim 3 or a pharmaceutically acceptable salt thereof, wherein the compound comprises:

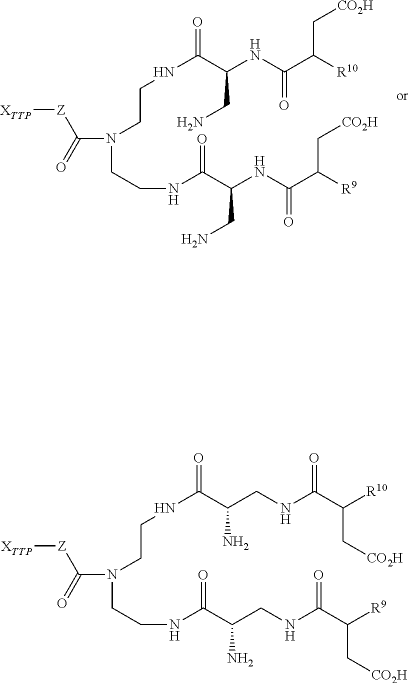

5. The compound of claim 1 or a pharmaceutically acceptable salt thereof, wherein R⁷ and R⁸ are:

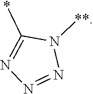

6. The compound of claim 5 or a pharmaceutically acceptable salt thereof, wherein:
R⁵ and R⁶ each comprise phenyl and CH₂O(CH₂CH₂O)ₙ, wherein n is 2;
R³ and R⁴ are each an amide;
R¹ and R² are each a C₂ alkyl; and
U is N or a phenyl.

7. The compound of claim 6 or a pharmaceutically acceptable salt thereof, wherein the compound comprises:

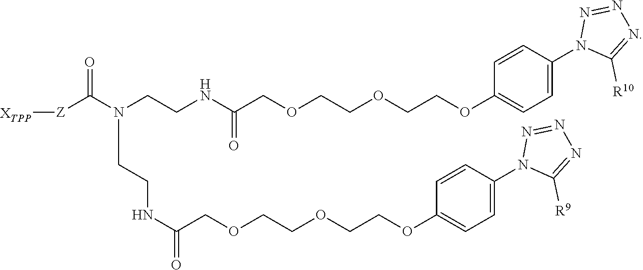

8. The compound of claim 1 or a pharmaceutically acceptable salt thereof, wherein R⁷ and R⁸ are each:

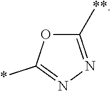

9. The compound of claim 10 or a pharmaceutically acceptable salt thereof, wherein:
R⁵ and R⁶ each comprise phenyl and CH₂O(CH₂CH₂O)ₙ, wherein n is 2;
R³ and R⁴ are each an amide;
R¹ and R² are each a C₂ alkyl; and
U is N or a phenyl.

10. The compound of claim 9 or a pharmaceutically acceptable salt thereof, wherein the compound comprises:

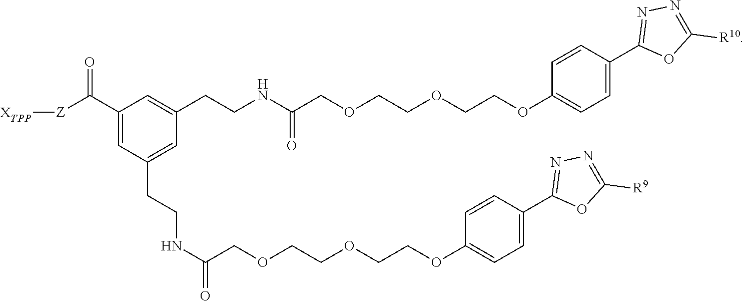

11. The compound of claim 1 or a pharmaceutically acceptable salt thereof, wherein:
R⁷ and R⁸ are each absent;
R⁵ and R⁶ are each C₁ alkyl;
R³ and R⁴ are each an amide;
R¹ and R² are each a C₂ alkyl; and
U is N or a phenyl.

12. The compound of claim 11 or a pharmaceutically acceptable salt thereof, wherein the compound comprises:

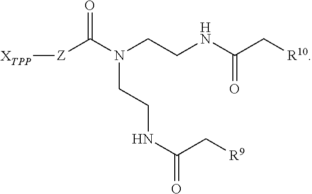

13. The compound of claim 1 or a pharmaceutically acceptable salt thereof, wherein the compound comprises:

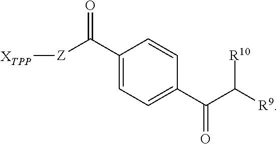

14. The compound of claim 1 or a pharmaceutically acceptable salt thereof, wherein Z comprises: (OCH₂CH₂)₂₄—NH—C(O)CH₂CH₂.

15. The compound of claim 1 or a pharmaceutically acceptable salt thereof, wherein Z comprises one to eight (2-[2-(2-amino-ethoxy)-ethoxy]-acetyl) moieties.

16. The compound of claim 1 or a pharmaceutically acceptable salt thereof, wherein Z comprises a peptide comprising any of SEQ ID NO:1182-1217.

17. The compound of claim 1 or a pharmaceutically acceptable salt thereof, wherein the therapeutic polypeptide comprises an agonist at one or more of the GIP, GLP-1, and glucagon receptors.

18. The compound of claim 1 or a pharmaceutically acceptable salt thereof, wherein the therapeutic polypeptide comprises:
(SEQ ID NO: 1218)
X₁X₂X₃X₄TX₆TSDX₁₀X₁₁X₁₂X₁₃LX₁₅X₁₆KAX₁₉X₂₀X₂₁FIX₂₄X₂₅LX₂₇X₂₈
X₂₉X₃₀X₃₁X₃₂X₃₃X₃₄X₃₅X₃₆X₃₇X₃₈X₃₉X₄₀X₄₁,
wherein:
X₁ is H, NMeY, or Y;
X₂ is Ac4c, Aib, αMeS, Iva, or D-Ala;
X₃ is Q, E, or H;
X₄ is G or D-Ala;
X₆ is αMeF(2F), F, or αMeF;
X₁₀ is 4-Pal, Y, or V;
X₁₁ is Aib, S, or αMeS;
X₁₂ is I or S;
X₁₃ is L or αMeL;
X₁₅ is D or E;
X₁₆ is K, E or Orn;
X₁₉ is A or Q;
X₂₀ is Aib, αMeL, Iva, or αMe4Pal;
X₂₁ is E, Q, D, or Orn;
X₂₄ is K, Q, E, D-Glu, or D-Gln;
X₂₅ is αMeY, W, or Y;
X₂₇ is I, L, or V;
X₂₈ K, E or A;
X₂₉ is Aib, G, S or Q;
X₃₀ is G, H or S;
X₃₁ is P, G, E, or Orn;
X₃₂ is absent, K, S or P;
wherein if X₃₂ is S or P, then X₃₃ is S;
wherein if X₃₃ is S, then X₃₄ is G or Aib;
wherein if X₃₄ is G or Aib, then X₃₅ is absent or A or Orn;
wherein if X₃₅ is A or Orn, then X₃₆ is absent or P;
wherein if X₃₆ is P, then X₃₇ is absent or P;
wherein if X₃₇ is P, then X₃₈ is absent, P or Ac3c;
wherein if X₃₈ is P or Ac3c, then X₃₉ is absent or S, Orn, or G;
wherein if X₃₉ is S, Orn, or G, then X₄₀ is absent, K, or G;
wherein if X₄₀ is K or G, then X₄₁ is absent or S or G;
wherein if X₃₂ is absent or K, then X₃₃ through X₄₁ are also absent;
wherein if X₃₅ is absent, then X₆₆ through X₄₁ are also absent;
wherein if X₃₆ is absent, then X₃₇ through X₄₁ are also absent;
wherein if X₃₇ is absent, then X₃₈ through X₄₁ are also absent;
wherein if X₃₈ is absent, then X₃₉ through X₄₁ are also absent;
wherein if X₃₉ is absent, then X₄₀ and X₄₁ are also absent;
wherein if X₄₀ is absent, then X₄₁ is absent.

19. The compound of claim 31 or a pharmaceutically acceptable salt thereof, wherein one of X₂₄, X₂₈, X₃₂ or X₄₀ is conjugated to Z.

20. The compound of claim 1 or a pharmaceutically acceptable salt thereof, wherein the therapeutic polypeptide comprises a 90% sequence identity to any of SEQ ID NOs: 1-186 or SEQ ID NOs: 996-1038.

21. The compound of claim 1 or a pharmaceutically acceptable salt thereof, wherein the therapeutic polypeptide comprises any of SEQ ID NOs: 1-186 or SEQ ID NOs: 996-1038.

22. The compound of claim 21 or a pharmaceutically acceptable salt thereof, wherein the fatty acid moiety is a C₁₆-C₂₂ fatty acid.

23. The compound of claim 22 or a pharmaceutically acceptable salt thereof, wherein the fatty acid is conjugated to the therapeutic polypeptide at position K17 via a linker.

24. The compound of claim 23 or a pharmaceutically acceptable salt thereof, wherein the linker comprises one to four amino acids.

25. The compound of claim 24 or a pharmaceutically acceptable salt thereof, wherein the linker comprises on or more of the amino acids Lys, εLys, Glu, γGlu or a combination thereof.

26. The compound of claim 23 or a pharmaceutically acceptable salt thereof, wherein the linker comprises one to eight (2-[2-(2-amino-ethoxy)-ethoxy]-acetyl) moieties.

27. The compound of claim 23 or a pharmaceutically acceptable salt thereof, wherein the linker and fatty acid comprise a structure of (εLys)ₐ(γGlu)_{b}-(2-[2-(2-amino-ethoxy)-ethoxy]-acetyl)_{c}-CO—(CH₂)p-CO₂H, wherein a is an integer between 0, 1 or 2, b is an integer between 0 or 1, c is an integer between 1 to 8, and p is an integer between 14 to 18.

28. The compound of claim 1 or a pharmaceutically acceptable salt thereof, wherein the therapeutic polypeptide conjugated to the fatty acid moiety comprises a 90% sequence identity to any of SEQ ID NOs:187-458 or SEQ ID NOs: 1039-1098.

29. The compound of claim 1 or a pharmaceutically acceptable salt thereof, wherein the therapeutic polypeptide conjugated to the fatty acid moiety comprises any of SEQ ID NOs:187-458 or SEQ ID NOs: 1039-1098.

30. The compound of claim 1, or a pharmaceutically acceptable salt thereof, wherein the Fc region is selected from any of SEQ ID NOs:935-936, 941-948; 1178-1181.

31. The compound of claim 1 or a pharmaceutically acceptable salt thereof, wherein the compound is selected from any of SEQ ID NOs: 459-934 or 1099-1177.

32. A compound of the formula: 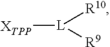
or a pharmaceutically acceptable salt thereof, wherein:
X_{TPP} is a therapeutic polypeptide, comprising:
(SEQ ID NO: 1219)
YAibEGTX₆TSDX₁₀X₁₁X₁₂X₁₃LX₁₅X₁₆KAQX₂₀X₂₁FIX₂₄X₂₅LX₂₇X₂₈X₂₉
X₃₀X₃₁X₃₂X₃₃X₃₄X₃₅X₃₆X₃₇X₃₈X₃₉X₄₀X₄₁
wherein:
X₆ is αMeF(2F), F, or αMeF;
X₁₀ is 4-Pal, Y, or V;
X₁₁ is Aib or S;
X₁₂ is I or S;
X₁₃ is L or αMeL;
X₁₅ is D or E;
X₁₆ is E or Orn;
X₂₀ is Aib, αMeL, or Iva;
X₂₁ is E, Q, D, or Orn;
X₂₄ is K, Q, E, or D-Glu;
X₂₅ is αMeY or Y;
X₂₇ is I, L, or V;
X₂₈ K, E or A;
X₂₉ is Aib, G, or Q;
X₃₀ is G or S;
X₃₁ is P, G, E, or Orn;
X₃₂ is absent, K, S, or P;
wherein if X₃₂ is S or P, then X₃₃ is S;
wherein if X₃₃ is S, then is X₃₄ is G or Aib;
wherein if X₃₄ is G or Aib, then X₃₅ is absent, A, or Orn;
wherein if X₃₅ is A or Orn, then X₃₆ is absent or P;
wherein if X₃₆ is P, then X₃₇ is absent or P;
wherein if X₃₇ is P, then X₃₈ is absent or P;
wherein if X₃₈ is P, then X₃₉ is absent, S, Orn, or G;
wherein if X₃₉ is S, Orn, or G, then X₄₀ is absent, K, or G;
wherein if X₄₀ is K or G, then X₄₁ is absent, S, or G;
wherein if X₃₂ is absent or K, then X₃₃ through X₄₁ are also absent;
wherein if X₃₅ is absent, then X₃₆ through X₄₁ are also absent;
wherein if X₃₆ is absent, then X₃₇ through X₄₁ are also absent;
wherein if X₃₇ is absent, then X₃₈ through X₄₁ are also absent;
wherein if X₃₈ is absent, then X₃₉ through X₄₁ are also absent;
wherein if X₃₉ is absent, then X₄₀ and X₄₁ are also absent;
wherein if X₄₀ is absent, then X₄₁ is absent;
L is a linker, conjugated to each of R⁹, R¹⁰, and the therapeutic polypeptide;
wherein R⁹ and R¹⁰ are independently an antibody fragment comprising an Fc region; and
wherein one of X₂₄, X₂₈, X₃₂ or X₄₀ is conjugated to the linker.

33. The compound of claim 32 or a pharmaceutically acceptable salt thereof, wherein the therapeutic polypeptide has dual agonist activity at the GIP and GLP-1 receptors.

34. The compound of claim 32, or a pharmaceutically acceptable salt thereof, comprising each of:
X₁ is Y; X₂ is Aib; X₃ is E; X₄ is G; X₆ is αMeF(2F); X₁₀ is 4-Pal or Y; X₁₁ is Aib or S; X₁₂ is I; X₁₃ is αMeL; X₁₅ is D; Xie is E or Orn; X₁ is Q; X₂₀ is Aib; X₂₁ is E; X₂₄ is Q, E, or D-Glu; X₂₅ is αMeY; X₂₇ is I; X₂₈ E; X₂₉ is Aib or G; X₃₀ is G or S; X₃₁ is P; X₃₂ is G or S;
wherein if X₃₂ is S then X₃₃ is S, X₃₄ is G, X₃₅ is A, X₃₆ is P, X₃₇ is P, X₃₈ is P, X₃₉ is S, X₄₀ is K, and X₄₁ is absent, and the linker is conjugated at X₄₀;
wherein if X₃₂ is G, then X₃₃ through X₄₁ are absent, and the linker is conjugated at X₃₂.

35. The compound of claim 34, or a pharmaceutically acceptable salt thereof, comprising at least one of: X₁₀ is Y or 4-Pal; X₁₁ is Aib or S; X₂₄ is Q, E, or D-Glu; and X₃₀ is G or S.

36. The compound of claim 34, or pharmaceutically acceptable salt thereof, comprising each of: X₁₀ is Y or 4-Pal; X₁₁ is Aib; X₂₄ is E; and X₃₀ is S; and wherein X₄₀ is K and is conjugated to the linker.

37. The compound of claim 34 or a pharmaceutically acceptable salt thereof, wherein the therapeutic polypeptide comprises a 90% sequence identity to any of SEQ ID NOs:1-92 or SEQ ID NOs:996-999.

38. The compound of claim 34, or a pharmaceutically acceptable salt thereof, wherein the therapeutic polypeptide comprises any of NOs:1-92 or SEQ ID NOs:996-999.

39. The compound of claim 34, or a pharmaceutically acceptable salt thereof, wherein the therapeutic polypeptide comprises any of SEQ ID NOs: 1, 2, 22, 43, 45, 48, 55, 56, 58, 59, 61, 66, 68 and 47.

40. The compound of claim 34, or a pharmaceutically acceptable salt thereof, wherein the therapeutic polypeptide is conjugated to a C₁₆-C₂₂ fatty acid moiety at position 17 via a second linker.

41. The compound of claim 40, or a pharmaceutically acceptable salt thereof, wherein the second linker comprises one or more of the amino acids Lys, εLys, Glu, γGlu or a combination thereof.

42. The compound of claim 40, or a pharmaceutically acceptable salt thereof, wherein the second linker comprises one to eight (2-[2-(2-amino-ethoxy)-ethoxy]-acetyl) moieties.

43. The compound of claim 40, or a pharmaceutically acceptable salt thereof, wherein the second linker and fatty acid comprises a structure of (εLys)ₐ(γGlu)_{b}-(2-[2-(2-amino-ethoxy)-ethoxy]-acetyl)_{c}-CO—(CH₂)ₚ—CO₂H, wherein a is an integer between 0, 1 or 2, b is an integer between 0 or 1, c is an integer between 1 to 8, and p is an integer between 14 to 18.

44. The compound of claim 40 or a pharmaceutically acceptable salt thereof, wherein the therapeutic polypeptide conjugated to the fatty acid moiety via the second linker comprises a 90% sequence identity to any of SEQ ID NOs:187-332 or SEQ ID NOs:1039-1042.

45. The compound of claim 40, or a pharmaceutically acceptable salt thereof, wherein the therapeutic polypeptide conjugated to the fatty acid moiety via the second linker comprises any of SEQ ID NOs:187-332 or SEQ ID NOs:1039-1042.

46. The compound of claim 40, or a pharmaceutically acceptable salt thereof, wherein the therapeutic polypeptide conjugated to the fatty acid moiety via the second linker comprises any of SEQ ID NOs: 187, 209, 235, 237, 241, 243, 257, 258, 260, 262, 263, 264, 266, 267, 275 or 277.

47. The compound of claim 32, or a pharmaceutically acceptable salt thereof, wherein the Fc region is selected from any of SEQ ID NOs:935-936, 941-948 or 1178-1181.

48. The compound of claim 32 or a pharmaceutically acceptable salt thereof, wherein the linker comprises: 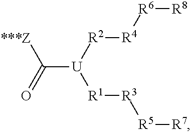
wherein:
Z comprises O, C(O), NH, C₁ to C₃₀ alkyl, (OCH₂CH₂)ₘ, [C(O)NH—CH₂CH₂OCH₂]ₘ, a peptide, or a combination thereof; wherein m is an integer between 1-30, and wherein (***) comprises a connection point to the therapeutic polypeptide;
U comprises C₁ to C₅ alkyl, N, phenyl, or phenyl carbonyl, or a combination thereof;
R¹ and R² are independently a covalent bond, or C₁ to C₅ alkyl;
R³ and R⁴ are absent or an amide;
R⁵ and R⁶ are independently absent or R⁵ and R⁶ independently comprise C₁ to C₅ alkyl, phenyl, CH₂O(CH₂CH₂O)o, or a combination thereof, wherein n is an integer between 1-5, wherein C₁ to C₅ alkyl is optionally substituted with one or more of NH₂, CH₂NH₂, and CH₂CH₂NH₂;
R⁷ and R⁸ are absent or independently selected from the group consisting of: 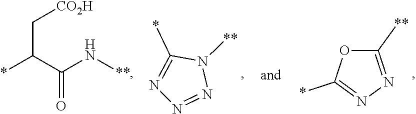
wherein:
(*) comprises a connection point to R⁹ or R¹⁰ and (**) comprises a connection point to R⁵ or R⁶.

49. The compound of claim 48, or pharmaceutically acceptable salt thereof, wherein R⁷ and R⁸ are:

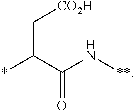

50. The compound of claim 49 or a pharmaceutically acceptable salt thereof, wherein the linker comprises: 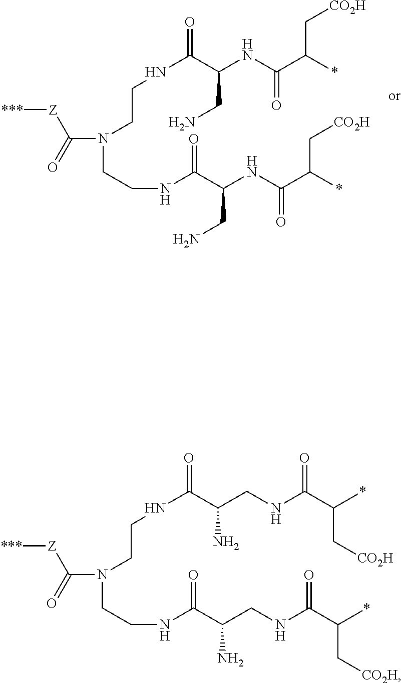
wherein (*) comprises a connection point to R⁹ or R¹⁰.

51. The compound of claim 48 or a pharmaceutically acceptable salt thereof, wherein R⁷ and R⁸ are:

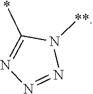

52. The compound of claim 51 or a pharmaceutically acceptable salt thereof, wherein the linker comprises: 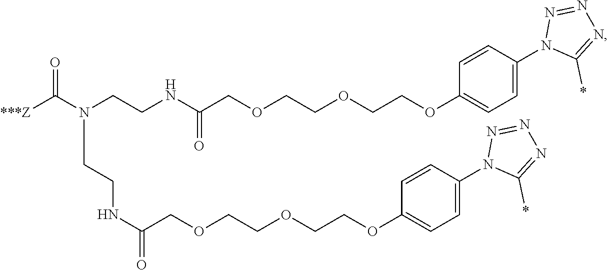
wherein (*) comprises a connection point to R⁹ or R¹⁰.

53. The compound of claim 48, wherein Z comprises: (OCH₂CH₂)₂₄—NH—C(O)CH₂CH₂.

54. The compound of claim 48 or a pharmaceutically acceptable salt thereof, wherein Z comprises one to eight (2-[2-(2-amino-ethoxy)-ethoxy]-acetyl) moieties.

55. The compound of claim 48, or a pharmaceutically acceptable salt thereof, wherein Z comprises a peptide comprising any of SEQ ID NO:1182-SEQ ID NO:1217.

56. The compound of claim 32 or a pharmaceutically acceptable salt thereof, wherein the compound is any of SEQ ID NOs:459-787 or SEQ ID NOs:1099-1100.

57. The compound of claim 32, or a pharmaceutically acceptable salt thereof, wherein the compound is any of SEQ ID NOs: 459, 490, 505, 585, 587, 594, 616, 622, 626, 627, 628, 630, 631, 639, 641, 642, 643, 645, 646, 662, 698, 699, 700 or 1100.

58. A compound of the formula: 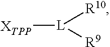
or a pharmaceutically acceptable salt thereof, wherein:
X_{TPP} is a therapeutic polypeptide, comprising:
(SEQ ID NO: 1220)
X₁X₂X₃X₄TX₆TSDX₁₀X₁₁X₁₂X₁₃LX₁₅X₁₆KAQX₂₀X₂₁FIX₂₄X₂₅LX₂₇X₂₈X₂₉
X₃₀X₃₁X₃₂X₃₃X₃₄X₃₅X₃₆X₃₇X₃₈X₃₉X₄₀X₄₁
wherein:
X₁ is H, NMeY, or Y;
X₂ is Ac4c, Aib, αMeS, Iva, or D-Ala;
X₃ is Q, E, or H;
X₄ is G or D-Ala;
X₆ is αMeF(2F), F, or αMeF;
X₁₀ is 4-Pal, Y, or V;
X₁₁ is Aib, S, or αMeS;
X₁₂ is I or S;
X₁₃ is L or αMeL;
X₁₅ is D or E;
X₁₆ is E or Orn;
X₂₀ is Aib, αMeL, Iva, or αMe4Pal;
X₂₁ is E, Q, D, or Orn;
X₂₄ is K, Q, E, D-Glu, or D-Gln;
X₂₅ is αMeY or Y;
X₂₇ is I, L, or V;
X₂₈ K, E or A;
X₂₉ is Aib, S, G, or Q;
X₃₀ is G, S or H;
X₃₁ is P, G, E, or Orn;
X₃₂ is absent, K, S or P;
wherein if X₃₂ is S or P, then X₃₃ is S;
wherein if X₃₃ is S, then X₃₄ is G or Aib;
wherein if X₃₄ is G or Aib, then X₃₅ is absent or A or Orn;
wherein if X₃₅ is A or Orn, then X₃₆ is absent or P;
wherein if X₃₆ is P, then X₃₇ is absent or P;
wherein if X₃₇ is P, then X₃₈ is absent or P;
wherein if X₃₈ is P, then X₃₉ is absent or S, Orn, or G;
wherein if X₃₉ is S, Orn, or G, then X₄₀ is absent, K, or G;
wherein if X₄₀ is K or G, then X₄₁ is absent or S or G;
wherein if X₃₂ or K is absent, then X₃₃ through X₄₁ are also absent;
wherein if X₃₅ is absent, then X₃₆ through X₄₁ are also absent;
wherein if X₃₆ is absent, then X₃₇ through X₄₁ are also absent;
wherein if X₃₇ is absent, then X₃₈ through X₄₁ are also absent;
wherein if X₃₈ is absent, then X₃₉ through X₄₁ are also absent;
wherein if X₃₉ is absent, then X₄₀ and X₄₁ are also absent;
wherein if X₄₀ is absent, then X₄₁ is absent;
L is a linker conjugated to each of R⁹, R¹⁰, and the therapeutic polypeptide;
wherein R⁹ and R¹⁰ are each an antibody fragment comprising an Fc region; and
one of X₂₄, X₃₂ or X₄₀ is conjugated to the linker.

59. The compound of claim 58, or a pharmaceutically acceptable salt thereof, wherein the therapeutic polypeptide has triple agonist activity at the GIP, GLP-1, and glucagon receptors.

60. The compound of claim 58, or a pharmaceutically acceptable salt thereof, comprising each of: X₁ is H or Y; X₂ is Aib; X₃ is Q; X₄ is G; X₆ is αMeF(2F); X₁₀ is Y or 4-Pal; X₁₁ is S or αMeS; X₁₂ is I; X₁₃ is αMeL; X₁₅ is D; X₂₀ is Aib or αMe4Pal; X₂₁ is E or Orn; X₂₄ is E or d-Glu; X₂₅ is αMeY; X₂₇ is L or I; X₂₈ is E; X₂₉—X₃₉ is GSPSSGAPPPS; and X₄₀ is K.

61. The compound of claim 60, or a pharmaceutically acceptable salt thereof, comprising at least one of: X₁₀ is Y or 4-Pal; X₂₄ is E or d-Glu; and X₃₀ is S.

62. The compound of claim 60, or a pharmaceutically acceptable salt thereof, comprising each of: X₁₀ is Y or 4-Pal; X₂₄ is E or d-Glu; X₃₀ is S and X₄₀ is K and is conjugated to the linker.

63. The compound of claim 58, or a pharmaceutically acceptable salt thereof, wherein the therapeutic polypeptide comprises a 90% sequence identity to any of SEQ ID NOs: 93-186 or SEQ ID NOs:1000-1038.

64. The compound of claim 58, or a pharmaceutically acceptable salt thereof, wherein the therapeutic polypeptide comprises any of SEQ ID NOs: 93-186 or SEQ ID NOs:1000-1038.

65. The compound of claim 58, or a pharmaceutically acceptable salt thereof, wherein the therapeutic polypeptide comprises any of SEQ ID NOs: 97, 113, 128, 130, 144, 171, 183.

66. The compound of claim 58, or a pharmaceutically acceptable salt thereof, wherein the therapeutic polypeptide is conjugated to a C₁₆-C₂₂ fatty acid moiety at position K17 via a second linker.

67. The compound of claim 66, or a pharmaceutically acceptable salt thereof, wherein the second linker comprises one or more of the amino acids Lys, εLys, Glu, γGlu or a combination thereof.

68. The compound of claim 66, or a pharmaceutically acceptable salt thereof, wherein the second linker comprises one to eight (2-[2-(2-amino-ethoxy)-ethoxy]-acetyl) moieties.

69. The compound of claim 66, or a pharmaceutically acceptable salt thereof, wherein the second linker and fatty acid comprises a structure of (εLys)ₐ(γGlu)_{b}-(2-[2-(2-amino-ethoxy)-ethoxy]-acetyl)_{c}-CO—(CH₂)ₚ—CO₂H, wherein a is an integer between 0, 1 or 2, b is an integer between 0 or 1, c is an integer between 1 to 8, and p is an integer between 14 to 18.

70. The compound of claim 66, or a pharmaceutically acceptable salt thereof, wherein the therapeutic polypeptide conjugated to the fatty acid moiety via the second linker comprises a 90% sequence identity to any of SEQ ID NOs:333-458 or SEQ ID NOs:1043-1098.

71. The compound of claim 66, or a pharmaceutically acceptable salt thereof, wherein the therapeutic polypeptide conjugated to the fatty acid moiety via the second linker comprises any of SEQ ID NOs:333-458 or SEQ ID NOs:1043-1098.

72. The compound of claim 66, or a pharmaceutically acceptable salt thereof, wherein the therapeutic polypeptide conjugated to the fatty acid moiety via the second linker comprises any of SEQ ID NOs: 337, 380, 395, 404, 423, 424, 426, 435, 447, 450, 467, 490 or 493.

73. The compound of claim 58, or a pharmaceutically acceptable salt thereof, wherein the Fc region is selected from any of SEQ ID NOs:935-936, 941-948 or 1178-1181.

74. The compound of claim 58, or a pharmaceutically acceptable salt thereof, wherein the linker comprises: 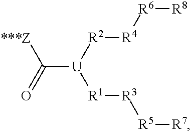
wherein:
Z comprises O, C(O), NH, C₁ to C₃₀ alkyl, (OCH₂CH₂)ₘ, [C(O)NH—CH₂CH₂OCH₂]ₘ, a peptide, or a combination thereof; wherein m is an integer between 1-30 and wherein (***) comprises a connection point to the therapeutic polypeptide;
U comprises C₁ to C₅ alkyl, N, phenyl, or phenyl carbonyl, or a combination thereof;
R¹ and R² are independently a covalent bond, or C₁ to C₅ alkyl;
R³ and R⁴ are absent or an amide;
R⁵ and R⁶ are independently absent or R⁵ and R⁶ independently comprise C₁ to C₅ alkyl; phenyl, CH₂O(CH₂CH₂O)o, or a combination thereof, wherein n is an integer between 1-5, wherein C₁ to C₅ alkyl is optionally substituted with one or more of NH₂, CH₂NH₂, and CH₂CH₂NH₂;
R⁷ and R⁸ are absent or independently selected from the group consisting of: 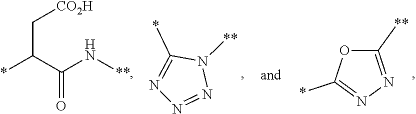
wherein:
(*) comprises a connection point to R⁹ or R¹⁰ and (**) comprises a connection point to R⁵ or R⁶.

75. The compound of claim 74, or a pharmaceutically acceptable salt thereof, wherein R⁷ and R⁸ are:

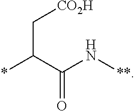

76. The compound of claim 75 or a pharmaceutically acceptable salts thereof, wherein the linker comprises: 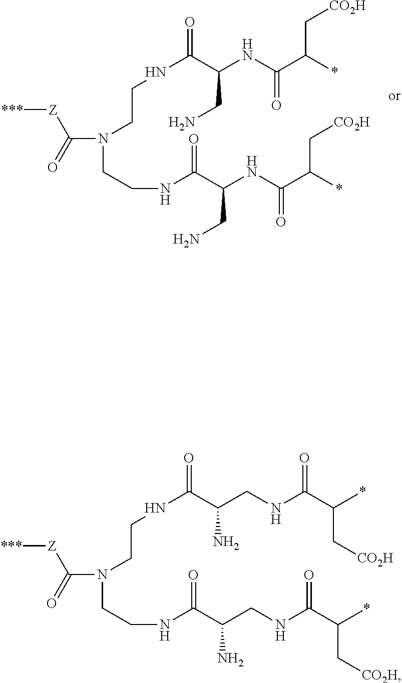
wherein (*) comprises a connection point to R⁹ or R¹⁰.

77. The compound of claim 74 or a pharmaceutically acceptable salt thereof, wherein R⁷ and R⁸ are:

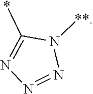

78. The compound of claim 74, or pharmaceutically acceptable salt thereof, wherein the linker comprises: 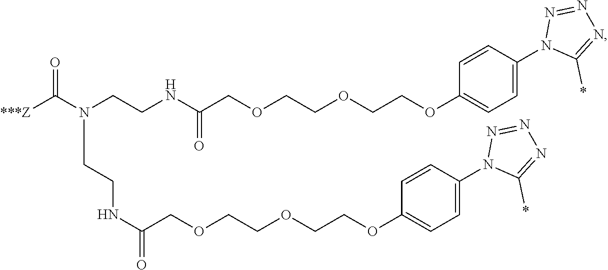
wherein (*) comprises a connection point to R⁹ or R₁₀.

79. The compound of claim 74, or a pharmaceutically acceptable salt thereof, wherein Z comprises: (OCH₂CH₂)₂₄—NH—C(O)CH₂CH₂.

80. The compound of claim 74, or a pharmaceutically acceptable salt thereof, wherein Z comprises one to eight (2-[2-(2-amino-ethoxy)-ethoxy]-acetyl) moieties.

81. The compound of claim 90, wherein Z comprises a peptide comprising any of SEQ ID NOs:1182-1217.

82. The compound of claim 74, or a pharmaceutically acceptable salt thereof, wherein the compound is any of SEQ ID NOs:788-934 or SEQ ID NOs: 1101-1177.

83. The compound of claim 74, or a pharmaceutically acceptable salt thereof, wherein the compound is any of SEQ ID NOs: 792, 843, 859, 869, 889, 891, 900, 912, 915, 1101, 1102, 1103, 1104, 1110, 1172, 1173, 1174 or 1175.

84. The compound of claim 1, or a pharmaceutically acceptable salt thereof, wherein the compound has a sufficiently extended duration of action to allow for dosing once monthly.

85. A pharmaceutical composition comprising the compound of claim 1, or a pharmaceutically acceptable salt thereof, and at least one pharmaceutically acceptable carrier, diluent, or excipient.

86. A method of treating a disease or condition, the method comprising a step of administering to an individual in need thereof an effective amount of a compound of claim 1, or a pharmaceutically acceptable salt thereof, once monthly.

87. The method of claim 104, wherein the disease or condition is selected from the group consisting of diabetes mellitus, obesity, chronic weight management, non-alcoholic fatty liver disease (NAFLD), non-alcoholic steatohepatitis (NASH), dyslipidemia, metabolic syndrome, chronic kidney disease (CKD), osteoarthritis (OA), obesity-related sleep apnea (OSA), polycystic ovary syndrome (PCOS), Parkinson's disease, Alzheimer's disease, heart failure (HF), hypertension (HT), peripheral arterial disease (PAD), metabolic syndrome, chronic lower back pain (CLBP), hepatic cirrhosis, polycystic ovary syndrome, and/or alcohol abuse disorder.
